(19) United States
(12) Patent Application Publication
(10) Pub. No.: US 2013/0310340 A1
(43) Pub. Date: Nov. 21, 2013

(54) METHOD OF TREATING MUSCULAR DEGRADATION

(71) Applicant: Rigel Pharmaceuticals, Inc., South San Francisco, CA (US)

(72) Inventors: Donald G. Payan, Hillsborough, CA (US); Esteban Masuda, Menlo Park, CA (US); Todd Kinsella, Redwood City, CA (US)

(73) Assignee: Rigel Pharmaceuticals, Inc., South San Francisco, CA (US)

(21) Appl. No.: 13/895,152

(22) Filed: May 15, 2013

Related U.S. Application Data
(60) Provisional application No. 61/647,982, filed on May 16, 2012.

(51) Int. Cl. A61K 31/519; A61K 45/06; A61K 31/541; A61K 31/506; A61K 31/554

(52) U.S. Cl. CPC A61K 31/519; A61K 31/506; A61K 31/554; A61K 31/541; A61K 45/06
USPC 514/80; 514/265.1; 514/210.21; 514/275; 514/211.08; 514/235.8; 514/252.18; 514/212.07; 514/233.8; 514/222.8; 514/249; 514/234.5; 514/230.5; 514/300; 514/228.5; 544/280; 544/323; 540/545; 544/122; 514/218; 514/256

(57) ABSTRACT

A method for treating muscular deterioration is disclosed. Also disclosed are embodiments of a compound, and compositions comprising the compound, for inhibiting muscular deterioriation, including atrophy, dystrophy, and cachexia, such as may result from ventilation.

### CROSS REFERENCE TO RELATED APPLICATION

This application claims the benefit of the earlier filing date of U.S. Provisional Application No. 61/647,982, filed on May 16, 2012, which is incorporated herein by reference in its entirety.

### FIELD

The present disclosure concerns embodiments of a method for treating muscular degradation, such as atrophy, dystrophic disorders, and cachectic states, with certain particular embodiments concerning treating ventilator-associated atrophy, using disclosed compounds and compositions comprising such compounds.

### BACKGROUND

The loss of muscle strength and tissue deterioration affects numerous individuals. In particular, atrophy involves a wasting away or deterioration of an organ, tissue, or body part typically caused by disease, injury, or lack of use. Many different types of atrophy are known. The primary diseases involve muscle atrophy, which is the wasting or loss of muscle tissue; multiple-system atrophy, which is a degenerative neurological disorder associated with the degeneration of nerve cells in the brain; and spinal muscular atrophy, which affects the spinal cord and nerves. Typically diseases that cause or result in atrophy affect one or more muscles within the human body. Similarly, dystrophic disorders also are typically caused by disease and have substantially the same effect as atrophy on muscle. Cachexia, as another example, involves weight loss, muscle atrophy, fatigue, and weakness, which cannot be reversed nutritionally. Individuals in cachectic states may be suffering from any number of diseases, such as cancer, AIDS, chronic obstructive lung disease, tuberculosis, multiple sclerosis, and the like.

Muscular atrophy or dystrophy also results from invasive mechanical ventilation. Typically, this type of ventilation is a required, lifesaving technique for subjects, particularly those with acute respiratory failure. Despite its lifesaving capabilities, controlled mechanical ventilation (CMV) often results in complications for the subject. In particular, CMV can result in the atrophy of the diaphragm muscle within a matter of hours following the onset of CMV. Other associated risks include ventilator-associated pneumonia, airway injury, gastrointestinal bleeding, barotrauma, and thromboembolism.

Quickly weaning a subject from a ventilator is important to prevent substantial or complete atrophy of the diaphragm muscle. Typically, weaning involves two phases: (1) readiness testing, wherein the subject is allowed to breathe without assistance (or with minimal ventilator support); and (2) progressive withdrawal, which involves gradual shifting between support from the ventilator and the subject's own breathing. While these methods may help decrease the amount of time that a subject is ventilated, there nevertheless exists a need for a more preventative approach to the initial onset of muscular deterioriation associated with CMV.

### SUMMARY

Disclosed embodiments include a method for treating disorders characterized by inappropriate or excessive degradation of muscle tissue, including atrophy, dystrophy, cachexia, and the like. One embodiment of the method comprises administering to a subject a compound in an amount, and at a time (for certain embodiments), effective to inhibit or prevent muscle degradation. In particular disclosed embodiments, the compound is a kinase inhibitor, such as a Janus kinase inhibitor. For certain particular embodiments, the compound is a muscle degradation inhibitory compound selected from a pyrimidine-containing compound, a pyridine-containing compound, or any and all combinations thereof. The compound can have any of the formulas disclosed herein, may be selected from any particular embodiments provided herein, as well as others within the scope of the present disclosure as would be recognized by a person of ordinary skill in the art.

For certain embodiments, administering comprises exposing the subject to at least a first dose of the compound, or composition comprising the compound. The method may further comprise administering a second dose (or more, such as a third dose, fourth dose, fifth dose, or so on), and may also comprise determining a therapeutic blood level of the compound in the subject, or a therapeutic metabolite blood level of the compound, in the subject, and thereafter adjusting the first dose to a second dose to optimize therapeutic effect. The method may also comprise monitoring muscular deterioration in the subject to ascertain the ability of the compound to inhibit or prevent atrophy, dystrophy, or cachexia.

A single compound may be administered once, it can be administered continuously, or it can be administered serially in plural administrations to the subject. Alternatively, the method may involve administering two or more compounds, such as serially or in combination, to the subject. In particular disclosed embodiments, two or more compounds may be administered serially and then in combination, or in combination and then serially. A second therapeutic agent may be administered prior to or subsequent to the one or more compounds.

One or more compounds may be administered as a pharmaceutical composition. For example, the composition may include an excipient (e.g. buffers, tonic agents, and the like), at least a first and a second kinase inhibitor, and/or an additional therapeutic agent, such as an analgesic, an antibiotic, an anticoagulant, an anti-delirium agent, an anti-inflammatory, a bronchodilator, a hypnotic, a sedative, a neuromuscular blocking agent, an antihistamine, or combinations thereof.

The compound, or composition comprising the compound, may be administered by any effective method, including by way of example and without limitation, oral, buccal, mucosal, sublingual, intravenous, intra-arterial, intramuscular, subcutaneous, intraperitoneal, intraarticular, infusion, intrathecal, intraurethral, topical, subdermal, transdermal, intranasal, inhalation, pulmonary tract, intratracheal, intraocular, ocular, intraaural, vaginal, and rectal. A currently preferred mode of administration is intravenous administration. The compound, or composition comprising the compound, also may be administered by any of these methods prophylactically.

A particular embodiment of the method concerns treating maladies associated with ventilator use, such as ventilator disuse atrophy and ventilator-induced diaphragmatic dysfunction (VIDD). The method comprises administering to a subject one or more compounds in an amount effective to reduce or inhibit injury of diaphragm muscle fibers. Administering may comprise exposing the subject to at least a first dosage of the one or more compounds, and may thereafter comprise adjusting the first dose to a second dose, such as a second dose further optimized to inhibit and/or prevent injury of diaphragm muscle fibers. The one or more compounds may be administered before the subject is ventilated, while the subject is ventilated, after the subject is ventilated, and particularly may be administered to the subject prior to, and/or while, weaning the subject off a ventilator. Typically, the compound or compounds, or composition comprising the compounds, is a kinase inhibitor, such as Janus kinase inhibitor. In particular disclosed embodiments, the compound may be a pyrimidine-containing compound, a pyridine-containing compound, or any and all combinations thereof. A single compound may be administered once, serially in plural administrations to the subject, two or more compounds may be administered either serially or in combination to the subject, and/or the one or more compounds may be administered as a pharmaceutical composition. The compounds typically are administered intravenously. The ability of the subject to breath independently typically is determined, and the ventilator is then removed from the subject.

Pharmaceutical compositions formulated for treating diseases characterized by atrophy, dystrophy, cachexia, etc., also are disclosed. Such compositions typically comprise a pyrimidine-containing compound, a pyridine-containing compound, or any and all combinations thereof. The composition may further comprise one or more excipients (e.g., buffers, tonic agents, and the like) and/or a second therapeutic agent, such as an analgesic, an antibiotic, an anticoagulant, an anti-delirium agent, an anti-inflammatory, a bronchodilator, a hypnotic, a sedative, a neuromuscular blocking agent, an antihistamine, or combinations thereof.

The foregoing and other objects, features, and advantages of the invention will become more apparent from the following detailed description, which proceeds with reference to the accompanying figures.

### BRIEF DESCRIPTION OF THE DRAWINGS

FIG. 1 is a graph (specific force [y-axis] versus frequency [Hz]) illustrating the effect of a disclosed embodiment of the compound on diaphragm contractility of ventilated rats.

FIG. 2 is a force-frequence curve (force in g/cm² versus frequence in Hz) illustrating in vitro diaphragm contractile properties of diaphragm strips taken from control animals (closed squares), animals receiving a single injection of leupeptin under CMV (open circles) and animals receiving a single injection of saline under CMV (closed circles).

FIG. 3 is a bar graph (diaphragm fiber CSA in μm² versus fiber type) illustrating the diaphragm cross-sectional area (CSA) of different fiber types (I, IIa, and IIx/b) in control animals (solid bars), leupeptin-CMV animals (open bars), and saline-CMV animals (hatched bars).

FIG. 4 is a bar graph (cathepsin B activity in nmol/mg protein/min) illustrating diaphragm cathepsin B activity in control animals (solid bar), leupeptin-CMV animals (open bar), and saline-CMV animals (hatched bar).

FIG. 5 is a graph (tetanic tension in g/cm² versus cathepsin B activity in nmol/mg protein/min) illustrating the correlation between diaphragm maximal tetanic tension and in vitro diaphragm cathepsin B activity.

FIG. 6 is a bar graph (calpain specific cleavage Spectrin in % C) illustrating the results from Western blot analysis of intact all-spectrin and calpain-cleaved αII-spectrin in the diaphragm of control animals (solid bar), leupeptin-CMV animals (open bar), and saline-CMV animals (hatched bar).

FIG. 7 is a graph (tetanic tension in g/cm² versus cleaved/intact Spectrin) illustrating the correlation between diaphragm maximal tetanic tension and calpain activity.

### DETAILED DESCRIPTION

### I. Terms and Definitions

As used herein, the following words and phrases are intended to have the meanings as set forth below, except to the extent that the context in which they are used indicates otherwise or they are expressly defined to mean something different.

The symbol “—” means a single bond, “═” means a double bond, “≡” means a triple bond. The symbol “ [symbol] ” refers to a group on a double-bond as occupying either position on the terminus of the double bond to which the symbol is attached; that is, the geometry, E- or Z-, of the double bond is ambiguous and both isomers are included. When a group is depicted removed from its parent formula, the “˜” symbol may be used at the end of the bond which was theoretically cleaved in order to separate the group from its parent structural formula.

When chemical structures are depicted or described, unless explicitly stated otherwise, all carbons are assumed to have hydrogen substitution to conform to a valence of four. For example, in the structure on the left-hand side of the schematic below there are nine hydrogen atoms implied. The nine hydrogen atoms are depicted in the right-hand structure. Sometimes a particular atom in a structure is described in textual formula as having a hydrogen or hydrogens as substitution (expressly defined hydrogen), for example, —CH₂CH₂—. It would be understood by one of ordinary skill in the art that the aforementioned descriptive techniques are common in the chemical arts to provide brevity and simplicity to description of otherwise complex structures.

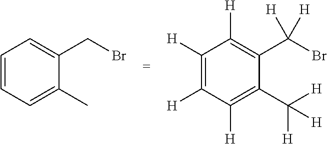

In this application, some ring structures are depicted generically and will be described textually. For example, in the schematic below if ring A is used to describe a phenyl, there are at most four hydrogens on ring A (when R is not H).

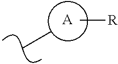

If a group R is depicted as “floating” on a ring system, as for example in the group:

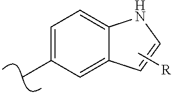

then, unless otherwise defined, a substituent R can reside on any atom of the fused bicyclic ring system, excluding the atom carrying the bond with the “˜” symbol, so long as a stable structure is formed. In the example depicted, the R group can reside on an atom in either the 5-membered or the 6-membered ring of the indolyl ring system.

When there are more than one such depicted “floating” groups, as for example in the formulae:

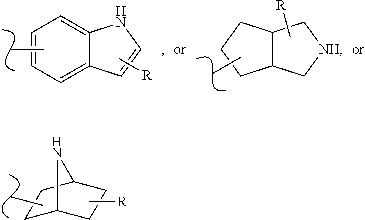

where there are two groups, namely, the R and the bond indicating attachment to a parent structure; then, unless otherwise defined, the “floating” groups can reside on any atoms of the ring system, again assuming each replaces a depicted, implied, or expressly defined hydrogen on the ring system and a chemically stable compound would be formed by such an arrangement.

When a group R is depicted as existing on a ring system containing saturated carbons, as for example in the formula:

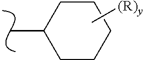

where, in this example, y can be more than one, assuming each replaces a currently depicted, implied, or expressly defined hydrogen on the ring; then, unless otherwise defined, two R's can reside on the same carbon. For example, a carbon atom of the ring system may comprise a geminal dimethyl group. In another example, two R's on the same carbon, including that same carbon, can form a ring, thus creating a spirocyclic ring (a “spirocyclyl” group) structure. Using the previous example, where two R's form, e.g. a piperidine ring in a spirocyclic arrangement with the cyclohexane, as for example in the formula:

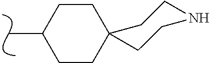

“Alkyl includes linear, branched, or cyclic hydrocarbon structures, and combinations thereof. Alkyl groups can be fully saturated or with one or more units of unsaturation, but not aromatic. Generally alkyl groups are defined by a subscript, either a fixed integer or a range of integers. For example, “C₈alkyl” includes n-octyl, iso-octyl, 3-octynyl, cyclohexenylethyl, cyclohexylethyl, and the like; where the subscript “8” designates that all groups defined by this term have a fixed carbon number of eight. In another example, the term “C₁₋₆alkyl” refers to alkyl groups having from one to six carbon atoms and, depending on any unsaturation, branches and/or rings, the requisite number of hydrogens. Examples of C₁₋₆alkyl groups include methyl, ethyl, vinyl, propyl, isopropyl, butyl, s-butyl, t-butyl, isobutyl, isobutenyl, pentyl, pentynyl, hexyl, cyclohexyl, hexenyl, and the like. When an alkyl residue having a specific number of carbons is named generically, all geometric isomers having that number of carbons are intended to be encompassed. For example, either “propyl” or “C₃alkyl” each include n-propyl, c-propyl, propenyl, propynyl, and isopropyl. Cycloalkyl is a subset of alkyl and includes cyclic hydrocarbon groups of from three to thirteen carbon atoms. Examples of cycloalkyl groups include c-propyl, c-butyl, c-pentyl, norbornyl, norbornenyl, c-hexenyl, adamantyl and the like. Alkyl refers to alkanyl, alkenyl, and alkynyl residues (and combinations thereof) —it is intended to include, e.g., cyclohexylmethyl, vinyl, allyl, isoprenyl, and the like. An alkyl with a particular number of carbons can be named using a more specific but still generic geometrical constraint, e.g. “C₃₋₆cycloalkyl” which means only cycloalkyls having between 3 and 6 carbons are meant to be included in that particular definition. Unless specified otherwise, alkyl groups, whether alone or part of another group, e.g. —C(O)alkyl, have from one to twenty carbons, that is C₁₋₂₀alkyl. In the example “—C(O)alkyl,” where there were no carbon count limitations defined, the carbonyl of the —C(O)alkyl group is not included in the carbon count, since “alkyl” is designated generically. But where a specific carbon limitation is given, e.g. in the term “optionally substituted C₁₋₂₀alkyl,” where the optional substitution includes “oxo” the carbon of any carbonyls formed by such “oxo” substitution are included in the carbon count since they were part of the original carbon count limitation. However, again referring to “optionally substituted C₁₋₂₀alkyl,” if optional substitution includes carbon-containing groups, e.g. —CH₂CO₂H, the two carbons in this group are not included in the C₁₋₂₀alkyl carbon limitation.

When a carbon number limit is given at the beginning of a term which itself comprises two terms, the carbon number limitation is understood as inclusive for both terms. For example, for the term “C₇₋₁₄arylalkyl,” both the “aryl” and the “alkyl” portions of the term are included in the carbon count, a maximum of 14 in this example, but additional substituent groups thereon are not included in the atom count unless they incorporate a carbon from the group's designated carbon count, as in the “oxo” example above. Likewise when an atom number limit is given, for example “6-14 membered heteroarylalkyl,” both the “heteroaryl” and the “alkyl” portion are included in the atom count limitation, but additional substituent groups thereon are not included in the atom count unless they incorporate a carbon from the group's designated carbon count. In another example, “C₄₋₁₀cycloalkylalkyl” means a cycloalkyl bonded to the parent structure via an alkylene, alkylidene or alkylidyne; in this example the group is limited to 10 carbons inclusive of the alkylene, alkylidene or alkylidyne subunit. As another example, the “alkyl” portion of, e.g. “C₇₋₁₄arylalkyl” is meant to include alkylene, alkylidene or alkylidyne, unless stated otherwise, e.g. as in the terms “C₇₋₁₄arylalkylene” or “C₆₋₁₀aryl-CH₂CH₂—.”

“Alkylene” refers to straight, branched and cyclic (and combinations thereof) divalent radical consisting solely of carbon and hydrogen atoms, containing no unsaturation and having from one to ten carbon atoms, for example, methylene, ethylene, propylene, n-butylene and the like. Alkylene is like alkyl, referring to the same residues as alkyl, but having two points of attachment and, specifically, fully saturated. Examples of alkylene include ethylene (—CH₂CH₂-propylene (—CH₂CH₂CH₂—), dimethylpropylene (—CH₂C(CH₃)₂CH₂—), cyclohexan-1,4-diyl and the like.

“Alkylidene” refers to straight, branched and cyclic (and combinations thereof) unsaturated divalent radical consisting solely of carbon and hydrogen atoms, having from two to ten carbon atoms, for example, ethylidene, propylidene, n-butylidene, and the like. Alkylidene is like alkyl, referring to the same residues as alkyl, but having two points of attachment and, specifically, at least one unit of double bond unsaturation. Examples of alkylidene include vinylidene (—CH═CH—), cyclohexylvinylidene (—CH═C(C₆H₁₃)—), cyclohexen-1,4-diyl and the like.

“Alkylidyne” refers to straight, branched and cyclic (and combinations thereof) unsaturated divalent radical consisting solely of carbon and hydrogen atoms having from two to ten carbon atoms, for example, propylid-2-ynyl, n-butylid-1-ynyl, and the like. Alkylidyne is like alkyl, referring to the same residues as alkyl, but having two points of attachment and, specifically, at least one unit of triple bond unsaturation.

Any of the above radicals” “alkylene,” “alkylidene” and “alkylidyne,” when optionally substituted, can contain alkyl substitution which itself can contain unsaturation. For example, 2-(2-phenylethynyl-but-3-enyl)-naphthalene (IUPAC name) contains an n-butylid-3-ynyl radical with a vinyl substituent at the 2-position of the radical. Combinations of alkyls and carbon-containing substitutions thereon are limited to thirty carbon atoms.

“Alkoxy” refers to the group —O-alkyl, where alkyl is as defined herein. Alkoxy includes, by way of example, methoxy, ethoxy, n-propoxy, isopropoxy, n-butoxy, t-butoxy, sec-butoxy, n-pentoxy, cyclohexyloxy, cyclohexenyloxy, cyclopropylmethyloxy, and the like.

“Haloalkyloxy” refers to the group —O-alkyl, where alkyl is as defined herein, and the alkyl group is substituted with one or more halogens. By way of example, a haloC₁₋₃alkyloxy” group includes —OCF₃, —OCF₂H, —OCHF₂, —OCH₂CH₂Br, —OCH₂CH₂CH₂I, —OC(CH₃)₂Br, —OCH₂Cl and the like.

“Acyl” refers to the groups —C(O)H, —C(O)alkyl, —C(O)aryl and —C(O)heterocyclyl.

“α-Amino Acids” refer to naturally occurring and commercially available α-amino acids and optical isomers thereof. Typical natural and commercially available α-amino acids are glycine, alanine, serine, homoserine, threonine, valine, norvaline, leucine, isoleucine, norleucine, aspartic acid, glutamic acid, lysine, ornithine, histidine, arginine, cysteine, homocysteine, methionine, phenylalanine, homophenylalanine, phenylglycine, ortho-tyrosine, meta-tyrosine, para-tyrosine, tryptophan, glutamine, asparagine, proline and hydroxyproline. A “side chain of an α-amino acid” refers to the radical found on the α-carbon of an α-amino acid as defined above, for example, hydrogen (for glycine), methyl (for alanine), benzyl (for phenylalanine), etc.

“Amino” refers to the group —NH₂.

“Amide” refers to the group —C(O)NH₂ or —N(H)acyl.

“Aryl” (sometimes referred to as “Ar”) refers to a monovalent aromatic carbocyclic group of, unless specified otherwise, from 6 to 15 carbon atoms having a single ring (e.g., phenyl) or multiple fused rings (e.g., naphthyl or anthryl), which condensed rings may or may not be aromatic (e.g., 2-benzoxazolinone, 2H-1,4-benzoxazin-3(4H)-one-7-yl, 9,10-dihydrophenanthrenyl, indanyl, tetralinyl, and fluorenyl and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic.

“Arylene” refers to an aryl that has at least two groups attached thereto. For a more specific example, “phenylene” refers to a divalent phenyl ring radical. A phenylene, thus can have more than two groups attached, but is defined by a minimum of two non-hydrogen groups attached thereto.

“Arylalkyl” refers to a residue in which an aryl moiety is attached to a parent structure via one of an alkylene, alkylidene, or alkylidyne radical. Examples include benzyl, phenethyl, phenylvinyl, phenylallyl and the like. When specified as “optionally substituted,” both the aryl, and the corresponding alkylene, alkylidene, or alkylidyne portion of an arylalkyl group can be optionally substituted. By way of example, “C₇₋₁₁arylalkyl” refers to an arylalkyl limited to a total of eleven carbons, e.g., a phenylethyl, a phenylvinyl, a phenylpentyl and a naphthylmethyl are all examples of a “C₇₋₁₁arylalkyl” group.

“Aryloxy” refers to the group —O-aryl, where aryl is as defined herein, including, by way of example, phenoxy, naphthoxy, and the like.

“Carboxyl,” “carboxy” or “carboxylate” refers to —CO₂H, salts thereof, or —C(O)O⁻.

“Carboxyl ester” or “carboxy ester” or “ester” refers to the group —CO₂alkyl, —CO₂aryl, —CO₂heteraryl, or —CO₂heterocyclyl.

“Carbonate” refers to the group —OCO₂alkyl, —OCO₂aryl or —OCO₂heterocyclyl.

“Carbamate” refers to the group —OC(O)NH₂, —N(H)carboxyl or —N(H)carboxyl ester.

“Cyano” or “nitrile” refers to the group —CN.

“Formyl” refers to the specific acyl group —C(O)H.

“Halo” or “halogen” refers to fluoro, chloro, bromo and iodo.

“Haloalkyl” and “haloaryl” refer generically to alkyl and aryl radicals that are substituted with one or more halogens, respectively. By way of example “dihaloaryl,” “dihaloalkyl,” “trihaloaryl” etc. refer to aryl and alkyl substituted with a plurality of halogens, but not necessarily a plurality of the same halogen; thus 4-chloro-3-fluorophenyl is a dihaloaryl group.

“Heteroalkyl” refers to an alkyl where one or more, but not all, carbons are replaced with a heteroatom. A heteroalkyl group has either linear or branched geometry. By way of example, a “2-6 membered heteroalkyl” is a group that can contain no more than 5 carbon atoms, because at least one of the maximum 6 atoms must be a heteroatom, and the group is linear or branched. Also, for the purposes of this invention, a heteroalkyl group always starts with a carbon atom, that is, although a heteroalkyl may contain one or more heteroatoms, the point of attachment to the parent molecule is not a heteroatom. A 2-6 membered heteroalkyl group includes, for example, —CH₂XCH₃, —CH₂CH₂XCH₃, —CH₂CH₂XCH₂CH₃, —C(CH₂)₂XCH₂CH₃ and the like, where X is O, NH, NC₁₋₆alkyl and S(O)₀₋₂, for example.

“Janus kinase (JAK) family” is a recognized family of non-receptor tyrosine kinases. Mammals have four (or more) members of this family, such as JAK1, JAK2, JAK3 and Tyrosine kinase 2 (TYK2).

“JAK inhibitor” refers to a compound that inhibits at least one member of the Janus kinase family. Methods for determining JAK inhibition are well known in the art and can be performed, for example, using kits or services commercially available from Ambit Biosciences, Invitrogen and others. Typically JAK inhibitors described herein have an 1050 for at least one member of the JAK family of less than about 10 μM, such as less than 5 μM, such as up to about 1 μM or less than about 100 nM.

“Perhalo” as a modifier means that the group so modified has all its available hydrogens replaced with halogens. An example would be “perhaloalkyl.” Perhaloalkyls include —CF₃, —CF₂CF₃, perchloroethyl and the like.

“Hydroxy” or “hydroxyl” refers to the group —OH.

“Heteroatom” refers to O, S, N, or P.

“Heterocyclyl” includes aromatic and non-aromatic ring systems and more specifically refers to a stable three- to fifteen-membered ring radical that consists of carbon atoms and from one to five heteroatoms. For purposes of this invention, the heterocyclyl radical can be a monocyclic, bicyclic or tricyclic ring system, which can include fused or bridged ring systems as well as spirocyclic systems; and the nitrogen, phosphorus, carbon or sulfur atoms in the heterocyclyl radical can be optionally oxidized to various oxidation states. In a specific example, the group —S(O)₀₋₂—, refers to —S-(sulfide), —S(O)— (sulfoxide), and —SO₂— (sulfone) linkages. For convenience, nitrogens, particularly but not exclusively, those defined as annular aromatic nitrogens, are meant to include their corresponding N-oxide form, although not explicitly defined as such in a particular example. Thus, for a compound having, for example, a pyridyl ring; the corresponding pyridyl-N-oxide is meant to be included in the presently disclosed compounds. In addition, annular nitrogen atoms can be optionally quaternized. “Heterocycle” includes heteroaryl and heteroalicyclyl, that is a heterocyclic ring can be partially or fully saturated or aromatic. Thus a term such as “heterocyclylalkyl” includes heteroalicyclylalkyls and heteroarylalkyls. Examples of heterocyclyl radicals include, but are not limited to, azetidinyl, acridinyl, benzodioxolyl, benzodioxanyl, benzofuranyl, carbazoyl, cinnolinyl, dioxolanyl, indolizinyl, naphthyridinyl, perhydroazepinyl, phenazinyl, phenothiazinyl, phenoxazinyl, phthalazinyl, pteridinyl, purinyl, quinazolinyl, quinoxalinyl, quinolinyl, isoquinolinyl, tetrazoyl, tetrahydroisoquinolyl, piperidinyl, piperazinyl, 2-oxopiperazinyl, 2-oxopiperidinyl, 2-oxopyrrolidinyl, 2-oxoazepinyl, azepinyl, pyrrolyl, 4-piperidonyl, pyrrolidinyl, pyrazolyl, pyrazolidinyl, imidazolyl, imidazolinyl, imidazolidinyl, dihydropyridinyl, tetrahydropyridinyl, pyridinyl, pyrazinyl, pyrimidinyl, pyridazinyl, oxazolyl, oxazolinyl, oxazolidinyl, triazolyl, isoxazolyl, isoxazolidinyl, morpholinyl, thiazolyl, thiazolinyl, thiazolidinyl, isothiazolyl, quinuclidinyl, isothiazolidinyl, indolyl, isoindolyl, indolinyl, isoindolinyl, octahydroindolyl, octahydroisoindolyl, quinolyl, isoquinolyl, decahydroisoquinolyl, benzimidazolyl, thiadiazolyl, benzopyranyl, benzothiazolyl, benzoxazolyl, furyl, diazabicycloheptane, diazapane, diazepine, tetrahydrofuryl, tetrahydropyranyl, thienyl, benzothieliyl, thiamorpholinyl, thiamorpholinyl sulfoxide, thiamorpholinyl sulfone, dioxaphospholanyl, and oxadiazolyl.

“Heteroaryl” refers to an aromatic group having from 1 to 10 annular carbon atoms and 1 to 4 annular heteroatoms. Heteroaryl groups have at least one aromatic ring component, but heteroaryls can be fully unsaturated or partially unsaturated. If any aromatic ring in the group has a heteroatom, then the group is a heteroaryl, even, for example, if other aromatic rings in the group have no heteroatoms. For example, 2H-pyrido[3,2-b][1,4]oxazin-3(4H)-one-7-yl, indolyl and benzimidazolyl are “heteroaryls.” Heteroaryl groups can have a single ring (e.g., pyridinyl, imidazolyl or furyl) or multiple condensed rings (e.g., indolizinyl, quinolinyl, benzimidazolyl or benzothienyl), where the condensed rings may or may not be aromatic and/or contain a heteroatom, provided that the point of attachment to the parent molecule is through an atom of the aromatic portion of the heteroaryl group. In one embodiment, the nitrogen and/or sulfur ring atom(s) of the heteroaryl group are optionally oxidized to provide for the N-oxide (N→O), sulfinyl, or sulfonyl moieties. Compounds described herein containing phosphorous, in a heterocyclic ring or not, include the oxidized forms of phosphorous. Heteroaryl groups are monocyclic, bicyclic, tricyclic or tetracyclic.

“Heteroaryloxy” refers to —O-heteroaryl.

“Heteroarylene” generically refers to any heteroaryl that has at least two groups attached thereto. For a more specific example, “pyridylene” refers to a divalent pyridyl ring radical. A pyridylene thus can have more than two groups attached, but is defined by a minimum of two non-hydrogen groups attached thereto.

“Heteroalicyclic” refers specifically to a non-aromatic heterocyclyl radical. A heteroalicyclic may contain unsaturation, but is not aromatic. As mentioned, aryls and heteroaryls are attached to the parent structure via an aromatic ring. So, e.g., 2H-1,4-benzoxazin-3(4H)-one-4-yl is a heteroalicyclic, while 2H-1,4-benzoxazin-3(4H)-one-7-yl is an aryl. In another example, 2H-pyrido[3,2-b][1,4]oxazin-3(4H)-one-4-yl is a heteroalicyclic, while 2H-pyrido[3,2-b][1,4]oxazin-3(4H)-one-7-yl is a heteroaryl.

“Heterocyclylalkyl” refers to a heterocyclyl group linked to the parent structure via e.g an alkylene linker, for example (tetrahydrofuran-3-yl)methyl- or (pyridin-4-yl)methyl

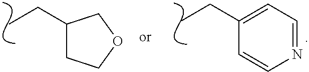

“Heterocyclyloxy” refers to the group —O-heterocycyl.

“Meta” for the purposes of this invention refers to the position of a substituent on a phenyl or a six-membered heteroaryl ring relative to another substituent on the ring; the relative position being 1,3-substitution. That is, starting from one substituent as being attached to a first atom of the six-membered ring and, counting atoms inclusive of the first atom, another substituent is on atom 3 of the six-membered ring, the substituents' relative orientation about the six-membered ring is “meta.”

“Nitro” refers to the group —NO₂.

“Ortho” for the purposes of this invention refers to the position of a substituent on a phenyl or a six-membered heteroaryl ring relative to another substituent on the ring; the relative position being 1,2-substitution. That is, starting from one substituent as being attached to a first atom of the six-membered ring and, counting atoms inclusive of the first atom, another substituent is on atom 2 of the six-membered ring, the substituents' relative orientation about the six-membered ring is “ortho.”

“Oxo” refers to a double bond oxygen radical, ═O.

“Oxy” refers to —O. radical (also designated as →O), that is, a single bond oxygen radical. By way of example, N-oxides are nitrogens bearing an oxy radical.

When a group with its bonding structure is denoted as being bonded to two partners; that is, a divalent radical, for example, —OCH₂—, then it is understood that either of the two partners can be bound to the particular group at one end, and the other partner is necessarily bound to the other end of the divalent group, unless stated explicitly otherwise. Divalent radicals are not to be construed as limited to the depicted orientation, for example “—OCH₂-” is meant to mean not only “—OCH₂-” as drawn, but also “—CH₂O—.”

“Optional” or “optionally” means that the subsequently described event or circumstance may or may not occur, and that the description includes instances where the event or circumstance occurs and instances in which it does not. One of ordinary skill in the art would understand that, with respect to any molecule described as containing one or more optional substituents, that only synthetically feasible compounds are meant to be included. “Optionally substituted” refers to all subsequent modifiers in a term, for example in the term “optionally substituted arylC₁₋₈alkyl,” optional substitution may occur on both the “C₁₋₈alkyl” portion and the “aryl” portion of the arylC₁₋₈alkyl group. Also by way of example, optionally substituted alkyl includes optionally substituted cycloalkyl groups. The term “substituted,” when used to modify a specified group or radical, means that one or more hydrogen atoms of the specified group or radical are each, independently of one another, replaced with the same or different substituent groups as defined below. Thus, when a group is defined as “optionally substituted” the definition is meant to encompass when the groups is substituted with one or more of the radicals defined below, and when it is not so substituted.

Substituent groups for substituting for one or more hydrogens (any two hydrogens on a single carbon can be replaced with ═O, ═NR⁹², ═N—OR⁹², ═N₂ or ═S) on saturated carbon atoms in the specified group or radical include (but are not limited to), unless otherwise specified, —R⁹³, halo, ═O, —OR⁹², —SR⁹², —N(R⁹⁴)₂, perhaloalkyl, —CN, —OCN, —SCN, —NO, —NO₂, ═N₂, —N₃, —SO₂R⁹², —SO₃⁻M⁺, —SO₃R⁹², —OSO₂R⁹², —OSO₃⁻M⁺, —OSO₃R⁹², —P(O)(O⁻)₂(M⁺)₂, —P(O)(O⁻)₂M²⁺, —P(O)(OR⁹²)O⁻M⁺, —P(O)(OR⁹²)₂, —C(O)R⁹², —C(S)R⁹², —C(NR⁹²)R⁹², CO₂⁻M⁺, —CO₂R⁹², —C(S)OR⁹², —C(O)N(R⁹⁴)², —C(NR⁹²)(R⁹⁴)₂, OC(O)R⁹², —OC(S)R⁹², —OCO₂⁻M⁺, —OCO₂R⁹², —OC(S)OR⁹², —NR⁹²C(O)R⁹², NR⁹²C(S)R⁹², —NR⁹²CO₂⁻M⁺, —NR⁹²CO₂R⁹², —NR⁹²C(S)OR⁹², —NR⁹²C(O)N(R⁹⁴)₂, —NR⁹²C(NR⁹²)R⁹² and —NR⁹²C(NR⁹²)N(R⁹⁴)₂, where R⁹³ is C₁₋₆alkyl, 3 to 10-membered heterocyclyl, 3 to 10-membered heterocyclylC₁₋₆alkyl, C₆₋₁₀aryl or C₆₋₁₀arylC₁₋₆alkyl; each R⁹² is independently for each occurrence hydrogen or R⁹⁴; each R⁹⁴ is independently for each occurrence R⁹² or alternatively, two R⁹⁴'s, taken together with the nitrogen atom to which they are bonded, form a 3- to 7-membered heteroalicyclyl which optionally includes from 1 to 4 of the same or different additional heteroatoms selected from O, N and S, of which N optionally has H or C₁-C₃alkyl substitution; and each M⁺ is a counter ion with a net single positive charge. Each M⁺ is independently for each occurrence, for example, an alkali ion, such as K⁺, Na⁺, Li⁺; an ammonium ion, such as ⁺N(R⁹³)₄; or an alkaline earth ion, such as [Ca²⁺]_{0.5}, [Mg²⁺]_{0.5}, or [Ba²⁺]_{0.5} (a “subscript 0.5 means e.g. that one of the counter ions for such divalent alkali earth ions can be an ionized form of a compound of the invention and the other a typical counter ion such as chloride, or two ionized compounds can serve as counter ions for such divalent alkali earth ions, or a doubly ionized compound can serve as the counter ion for such divalent alkali earth ions). As specific examples, —N(R⁹⁴)₂ is meant to include —NH₂, —NH-alkyl, —NH-pyrrolidin-3-yl, N-pyrrolidinyl, N-piperazinyl, 4N-methyl-piperazin-1-yl, N-morpholinyl and the like.

Substituent groups for replacing hydrogens on unsaturated carbon atoms in groups containing unsaturated carbons are, unless otherwise specified, —R⁹³, halo, —O⁻M⁺, —OR⁹², —SR⁹², —S⁻M⁺, —N(R⁹⁴)₂, perhaloalkyl, —CN, —OCN, —SCN, —NO, —NO₂, —N₃, —SO₂R⁹², —SO₃⁻M⁺, —SO₃R⁹², —OSO₂R⁹², —OSO₃⁻M⁺, —OSO₃R⁹², —PO₃⁻²(M⁺)₂, —PO₃⁻²M²⁺, —P(O)(OR⁹²)O⁻M⁺, —P(O)(OR⁹²)₂, —C(O)R⁹²—C(NR⁹²)R⁹², —CO₂M⁺, —CO₂R⁹², —C(S)OR⁹², —C(O)NR⁹⁴R⁹⁴, —C(NR⁹²)N(R⁹⁴)₂, —OC(O)R⁹², —OC(S)R⁹², —OCO₂⁻M⁺, —OCO₂R⁹², —OC(S)OR⁹², —NR⁹²C(O)R⁹², —NR⁹²C(S)R⁹², —NR⁹²CO₂⁻M⁻, —NR⁹²CO₂R⁹², —NR⁹²C(S)OR⁹², —NR⁹²C(O)N(R⁹⁴)₂, —NR⁹²C(NR⁹²)R⁹² and —NR⁹²C(NR⁹²)N(R⁹⁴)₂, where R⁹³, R⁹², R⁹⁴ and M⁺ are as previously defined, provided that in case of substituted alkene or alkyne, the substituents are not —O⁻M⁺, —OR⁹², —SR⁹², or —S⁻M⁺.

Substituent groups for replacing hydrogens on nitrogen atoms in groups containing such nitrogen atoms are, unless otherwise specified, —R⁹³, —O⁻M⁺, —OR⁹², —SR⁹², —N(R⁹⁴)₂, perhaloalkyl, —CN, —NO, —NO₂, —S(O)₂R⁹², —SO₃M⁺, —SO₃R⁹², —OS(O)₂R⁹², —OSO₃⁻M⁺, —OSO₃R⁹², —PO₃²⁻(M⁺)₂, —PO₃²⁻M²⁺, —P(O)(OR⁹²)O⁻M⁺, —P(O)(OR⁹²)(OR⁹²), —C(O)R⁹², —C(S)R⁹², —C(NR⁹²)R⁹², —CO₂R⁹², —C(S)OR⁹², —C(O)NR⁹⁴R⁹⁴, —C(NR⁹²)NR⁹⁴R⁹⁴, —OC(O)R⁹², —OC(S)R⁹², —OCO₂R⁹², —OC(S)OR⁹², —NR⁹²C(O)R⁹², —NR⁹²C(S)R⁹², —NR⁹²CO₂R⁹², —NR⁹²C(S)OR⁹², —NR⁹²C(O)N(R⁹⁴)₂, —NR⁹²C(NR⁹²)R⁹² and —NR⁹²C(NR⁹²)N(R⁹⁴)₂, where R⁹³, R⁹², R⁹⁴ and M⁺ are as previously defined.

In one embodiment, a group that is substituted has 1, 2, 3, or 4 substituents, 1, 2, or 3 substituents, 1 or 2 substituents, or 1 substituent.

In such case that the language permits multiple substitutions, the maximum number of such iterations of substitution is three.

“Sulfonamide” refers to the group —SO₂NH₂, —N(H)SO₂H, —N(H)SO₂alkyl, —N(H)SO₂aryl, or —N(H)SO₂heterocyclyl.

“Sulfonyl” refers to the group —SO₂H, —SO₂alkyl, —SO₂aryl, or —SO₂heterocyclyl.

“Sulfanyl” refers to the group: —SH, —S-alkyl, —S-aryl, or —S-heterocyclyl.

“Sulfinyl” refers to the group: —S(O)H, —S(O)alkyl, —S(O)aryl or —S(O)heterocyclyl.

“Suitable leaving group” is defined as the term would be understood by one of ordinary skill in the art; that is, a group on a carbon, where upon reaction a new bond is to be formed, the carbon loses the group upon formation of the new bond. A typical example employing a suitable leaving group is a nucleophilic substitution reaction, e.g., on a sp³ hybridized carbon (SN₂ or SN₁), e.g. where the leaving group is a halide, such as a bromide, the reactant might be any suitable nucleophile sufficient to react and displace bromide as a leaving group. Another typical example of such a reaction is a nucleophilic aromatic substitution reaction (SNAr). Another example is an insertion reaction (for example by a transition metal) into the bond between an aromatic reaction partner bearing a leaving group followed by reductive coupling. “Suitable leaving group” is not limited to such mechanistic restrictions. Examples of suitable leaving groups include halogens, optionally substituted aryl or alkyl sulfonates, phosphonates, azides and —S(O)₀₋₂R where R is, for example optionally substituted alkyl, optionally substituted aryl, or optionally substituted heteroaryl. A person of ordinary skill in the art of organic synthesis will readily identify suitable leaving groups to perform a desired reaction under different reaction.

“Stereoisomer” and “stereoisomers” refer to compounds that have the same atomic connectivity but different atomic arrangement in space. Stereoisomers include cis-trans isomers, E and Z isomers, enantiomers and diastereomers. Compounds of the invention, or their pharmaceutically acceptable salts can contain one or more asymmetric centers and can thus give rise to enantiomers, diastereomers, and other stereoisomeric forms that can be defined, in terms of absolute stereochemistry, as (R)- or (S)- or, as (D)- or (L)- for amino acids. The present invention is meant to include all such possible isomers, as well as their racemic and optically pure forms. Optically active (+) and (−), (R)- and (S)-, or (D)- and (L)- isomers can be prepared using chiral synthons, chiral reagents, or resolved using conventional techniques, such as by: formation of diastereoisomeric salts or complexes which can be separated, for example, by crystallization; via formation of diastereoisomeric derivatives, which can be separated, for example, by crystallization; selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. If a desired enantiomer is converted into another chemical entity by one of the separation procedures described above, a further step may be required to liberate the desired enantiomeric form. Alternatively, specific enantiomer can be synthesized by asymmetric synthesis using optically active reagents, substrates, catalysts or solvents, or by converting on enantiomer to the other by asymmetric transformation. For a mixture of enantiomers, enriched in a particular enantiomer, the major component enantiomer can be further enriched (with concomitant loss in yield) by recrystallization.

When the compounds described herein contain olefinic double bonds or other centers of geometric asymmetry, and unless specified otherwise, it is intended that the compounds include both E and Z geometric isomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art will recognize that other tautomeric ring atom arrangements are possible and within the scope of the term.

“Para” for the purposes of this invention refers to the position of a substituent on a phenyl or a six-membered heteroaryl ring relative to another substituent on the ring; the relative position being 1,4-substitution. That is, starting from one substituent as being attached to a first atom of the six-membered ring and, counting atoms inclusive of the first atom, another substituent is on atom 4 of the six-membered ring, the substituents' relative orientation about the six-membered ring is “para.” For example compound L, depicted below, has a methyl group “para” to N2 of the pyrimidinediamine; compound M also has a “para” methyl group.

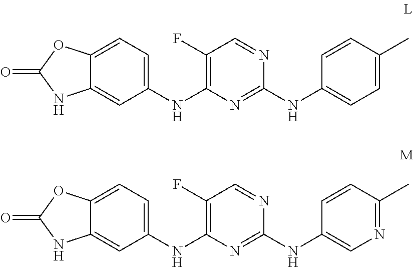 [SMILES: Cc1ccc(Nc2ncc(F)c(Nc3ccc4oc(=O)[nH]c4c3)n2)cc1 ; Cc1ccc(Nc2ncc(F)c(Nc3ccc4oc(=O)[nH]c4c3)n2)cn1]

“Patient” or “Subject” refers to mammals and other animals, particularly humans. Thus the methods are applicable to both human therapy and veterinary applications. In one embodiment the patient or subject is a mammal. In another embodiment the patient or subject is a human.

“Pharmaceutically acceptable salt” refers to pharmaceutically acceptable salts of a compound, which salts are derived from a variety of organic and inorganic counter ions well known in the art including, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate, and the like. Pharmaceutically acceptable acid addition salts are those salts that retain the biological effectiveness of the free bases while formed by acid partners that are not biologically or otherwise undesirable, e.g., inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid and the like. Pharmaceutically acceptable base addition salts include those derived from inorganic bases such as sodium, potassium, lithium, ammonium, calcium, magnesium, iron, zinc, copper, manganese, aluminum salts and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19, which is incorporated herein by reference.) Additional examples of suitable salts, without limitation, include citrate salts and xinafoate salts.

“Pharmaceutically effective amount” and “therapeutically effective amount” refer to an amount of a compound sufficient to treat a specified disorder or disease or one or more of its symptoms and/or to prevent the occurrence of the disease or disorder. The amount of a compound which constitutes a “therapeutically effective amount” will vary depending on the compound, the disease state and its severity, the age of the subject to be treated, and the like. A therapeutically effective amount can be determined routinely by one of ordinary skill in the art.

“Prodrug” refers to compounds that are transformed in vivo to yield the parent compound, for example, by hydrolysis in the gut or enzymatic conversion in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) where the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention can be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more polar molecule such as by oxidation, reduction, or hydrolysis, or to a conjugate (see Goodman and Gilman, “The Pharmacological Basis of Therapeutics” 12^{th} Ed., Pergamon Press, Gilman et al. (eds), 1990 which is herein incorporated by reference). The metabolite of a compound described herein or its salt can itself be a biologically active compound in the body. While a prodrug described herein would meet this criteria, that is, form a described biologically active parent compound in vivo, “metabolite” is meant to encompass those compounds not contemplated to have lost a progroup, but rather all other compounds that are formed in vivo upon administration of a compound of the invention which are biologically active. Thus one aspect disclosed compounds specifically contemplated herein is a metabolite of a compound described herein. Biologically active compounds inherently formed as a result of practicing methods of the invention, are contemplated and disclosed herein. “Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. The compounds described herein can exist in unsolvated as well as solvated forms with solvents, pharmaceutically acceptable or not, such as water, ethanol, and the like. Solvated forms of the presently disclosed compounds are contemplated herein and are encompassed by the invention, at least in generic terms.

“Muscular deterioration” as used herein covers any injury, damage, structural abnormality, or wasting away of any muscle within a subject. As used herein, muscular deterioration and muscular degradation are used interchangeably. Particular examples of muscular deterioration include muscular atrophy, muscular dystrophy, or cachexia state of muscle.

“Second Therapeutic (Agent)” as used herein concerns any additional compound, drug, or formulation that can be used with, either serially, in combination, or serially and in combination in any order, disclosed embodiments of the compound described here, particularly those agents used to aid in ventilating a subject. Particular examples of a second therapeutic agents are disclosed herein.

“Treating” or “treatment” as used herein covers the treatment of the disease or condition of interest in a mammal, preferably a human, having the disease or condition of interest, and includes:

(i) preventing the disease or condition from occurring in a mammal, in particular, when such mammal is predisposed to the condition but has not yet been diagnosed as having it;

(ii) inhibiting the disease or condition, for example, arresting or slowing its development;

(iii) relieving the disease or condition, for example, causing regression of the disease or condition or a symptom thereof; or

(iv) stabilizing the disease or condition.

As used herein, the terms “disease” and “condition” can be used interchangeably or can be different in that the particular malady or condition may not have a known causative agent (so that etiology has not yet been worked out) and it is therefore not yet recognized as a disease but only as an undesirable condition or syndrome, where a more or less specific set of symptoms has been identified by clinicians.

Similarly, it is understood that the above definitions are not intended to include impermissible substitution patterns (e.g., methyl substituted with 5 fluoro groups). Such impermissible substitution patterns are easily recognized by a person having ordinary skill in the art.

### II. Compounds and Compositions Thereof

A. Compounds

Disclosed herein are compounds that may be used to treat muscular degradation. In particular disclosed embodiments, the compound may be a kinase inhibitor, such as a Janus kinase inhibitor. The compound may be a pyrimidine- or pyridine-containing compound. The present disclosure incorporates herein by reference each compound disclosed in the following applications: WO2003/048162, WO2001/042246, WO2010/039939, WO2007/070514; WO2009/114512, WO2009/049028, U.S. Pat. No. 8,088,764, US2007/0135461, US2008/0312259, WO 2012/015972, WO2010/085684 (PCT/US2010/021856), US2009/0258864, U.S. Pat. No. 8,188,281, U.S. Pat. No. 8,163,917, and U.S. Pat. No. 8,129,399.

In particular disclosed embodiments, the compound is a pyrimidine-containing compound having a Formula I:

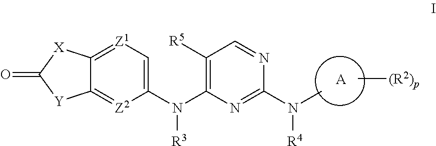

where:

X and Y are each independently O, S, S(O), SO₂ or NR¹;

each R¹ is independently for each occurrence H, optionally substituted C₁₋₆alkyl, C(O)—C₁₋₆alkyl, CO₂—C₁₋₆alkyl or R⁵⁰;

each R⁵⁰ is —C(R⁹)₂-A-R¹⁰, where A is O or S;

each R⁹ is independently for each occurrence H, optionally substituted C₁₋₆alkyl, optionally substituted C₆₋₁₀aryl or optionally substituted C₇₋₁₆arylalkyl; or alternatively, two R⁹, together with the carbon to which they are attached, form an optionally substituted C₃₋₈cycloalkyl group or an optionally substituted 3-8 membered heteroalicyclyl;

R¹⁰ is R^{a} or —P(O)(OR¹¹)₂;

each R¹¹ is independently for each occurrence R^{a} or a monovalent cationic group; or two R¹¹, together with the atoms to which they are attached, form a 4-8 membered cyclic phosphate group, or two R¹¹ together represent a divalent cationic group;

ring A is a C₆₋₁₀aryl or a 5-10 membered heteroaryl;

each R² is independently for each occurrence H, R^{e}, R^{b}, R^{e} substituted with one or more of the same or different R^{a} and/or R^{b}, —OR^{e} substituted with one or more of the same or different R^{a} and/or R^{b}, —SR^{e} substituted with one or more of the same or different R^{a} and/or R^{b}, —C(O)R^{e} substituted with one or more of the same or different R^{a} and/or R^{b}, —N(R^{a})R^{e} where R^{e} is substituted with one or more of the same or different R^{a} and/or R^{b}, —S(O)₂R^{e} substituted with one or more of the same or different R^{a} and/or R^{b}, —N(R^{a})—S(O)₂R^{e} where R^{e} is substituted with one or more of the same or different R^{a} and/or R^{b}, —B(OR^{a})₂, —B(N(R^{c})₂)₂, —(C(R^{a})₂)ₘ—R^{b}, —O—(C(R^{a})₂)ₘ—R^{b}, —S—(C(R^{a})₂)ₘ—R^{b}, —N(R^{a})—(C(R^{a})₂)ₘ—R^{b}, —O—(CH₂)ₘ—CH((CH₂)ₘR^{b})R^{b}, —C(O)N(R^{a})—(C(R^{a})₂)ₘ—R^{b}, —O—(C(R^{a})₂)ₘ—C(O)N(R^{a})—(C(R^{a})₂)ₘ—R^{b}, —N((C(R^{a})₂)ₘR^{b})₂, —S—(C(R^{a})₂)ₘ—C(O)N(R^{a})—(C(R^{a})₂)ₘ—R^{b}, —N(R^{a})—C(O)—N(R^{a})—(C(R^{a})₂)ₘ—R^{b}, —N(R^{a})—C(O)—(C(R^{a})₂)ₘ—C(R^{a})(R^{b})₂ or —N(R^{a})—(C(R^{a})₂)ₘ—C(O)—N(R^{a})—(C(R^{a})₂)ₘ—R^{b};

each R^{a} is independently for each occurrence H, deuterium, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-10 membered heteroalicyclyl, 4-11 membered heteroalicyclylalkyl, 5-15 membered heteroaryl or 6-16 membered heteroarylalkyl;

each R^{b} is independently for each occurrence ═O, —OR^{a}, —O—(C(R^{a})₂)ₘ—OR^{a}, haloC₁₋₃alkyloxy, ═S, —SR^{a}, ═NR^{a}, ═NOR^{a}, —N(R^{c})₂, halo, —CF₃, —CN, —NC, —OCN, —SCN, —NO, —NO₂; ═N₂, —N₃, —S(O)R^{a}, —S(O)₂R^{a}, —SO₃R^{a}, —S(O)N(R^{c})₂, —S(O)₂N(R^{c})₂, —OS(O)R^{a}, —OS(O)₂R^{a}, —OSO₃R^{a}, —OS(O)₂N(R^{c})₂, —C(O)R^{a}, —CO₂R^{a}, —C(O)N(R^{c})₂, —C(NR^{a})—N(R^{c})₂, —C(NOH)—R^{a}, —C(NOH)—N(R^{c})₂, —OC(O)R^{a}, —OC(O)OR^{a}, —OC(O)N(R^{c})₂, —OC(NH)—N(R^{c})₂, —OC(NR^{a})—N(R^{c})₂, —N(R^{a})—S(O)₂H, —[N(R^{a})C(O)]ₙR^{a}, —[N(R^{a})C(O)]ₙOR^{a}, —[N(R^{a})C(O)]ₙN(R^{c})₂ or —[N(R^{a})C(NR^{a})]ₙ—N(R^{c})₂;

each R^{c} is independently for each occurrence R^{a}, or, alternatively, two R^{c} are taken together with the nitrogen atom to which they are bonded to form a 3 to 10-membered heteroalicyclyl or a 5-10 membered heteroaryl which may optionally include one or more of the same or different additional heteroatoms and which is optionally substituted with one or more of the same or different R^{a} and/or R^{d} groups;

each R^{d} is ═O, —OR^{a}, haloC₁₋₃alkyloxy, C₁₋₆alkyl, ═S, —SR^{a}, ═NR^{a}, ═NOR^{a}, —N(R^{a})₂, halo, —CF₃, —CN, —NC, —OCN, —SCN, —NO, —NO₂, ═N₂, —N₃, —S(O)R^{a}, —S(O₂)R^{a}, —SO₃R^{a}, —S(O)N(R^{a})₂, —S(O)₂N(R^{a})₂, —OS(O)R^{a}, —OS(O)₂R^{a}, —OSO₃R^{a}, —OS(O)₂N(R^{a})₂, —C(O)R^{a}, —CO₂R^{a}, —C(O)N(R^{a})₂, —C(NR^{a})N(R^{a})₂, —C(NOH)R^{a}, —C(NOH)N(R^{a})₂, —OCO₂R^{a}, —OC(O)N(R^{a})₂, —OC(NR^{a})N(R^{a})₂, —[N(R^{a})C(O)]ₙR^{a}, —(C(R^{a})₂)ₙ—OR^{a}, —N(R^{a})—S(O)₂R^{a}, —C(O)—C₁₋₆haloalkyl, —S(O)₂C₁₋₆haloalkyl, —OC(O)R^{a}, —O(C(R^{a})₂)ₘ—OR^{a}, —S(C(R^{a})₂)ₘ—OR^{a}, —N(R^{a})C₁₋₆haloalkyl, —P(O)(OR^{a})₂, —N(R^{a})—(C(R^{a})₂)ₘ—OR^{a}, —[N(R^{a})C(O)]ₙOR^{a}, —[N(R^{a})C(O)]ₙN(R^{a})₂, —[N(R^{a})C(NR^{a})]ₙN(R^{a})₂ or —N(R^{a})C(O)C₁₋₆haloalkyl; or two R^{d}, taken together with the atom or atoms to which they are attached, combine to form a 3-10 membered partially or fully saturated mono or bicyclic ring, optionally containing one or more heteroatoms and optionally substituted with one or more R^{a};

each R^{e} is independently for each occurrence C₁₋₆alkyl, C₃₋₈cycloalkyl, C₄₋₁₁ cycloalkylalkyl, C₆₋₁₀aryl, C₇₋₁₆arylalkyl, 2-6 membered heteroalkyl, 3-10 membered heteroalicyclyl, 4-11 membered heteroalicyclylalkyl, 5-15 membered heteroaryl or 6-16 membered heteroarylalkyl;

p is 0, 1, 2, 3 or 4;

each m is 1, 2 or 3;

each n is 0, 1, 2 or 3;

or two R² groups, taken together with the atom or atoms to which they are attached, combine to form a 4-10 membered partially or fully saturated mono or bicyclic ring, optionally containing one or more heteroatoms and optionally substituted with one or more R^{a} and/or R^{b};

Z¹ and Z² are each independently CH, CR² or N;

R³ is H, optionally substituted C₁₋₆alkyl or R⁵⁰;

R⁴ is H, optionally substituted C₁₋₆alkyl or R⁵⁰; and

R⁵ is halo, —CN, optionally substituted C₁₋₆alkyl, alkynyl, hydroxy, optionally substituted C₁₋₆alkoxy, nitro, —N(R^{a})₂, —C(O)N(R^{a})₂, —CO₂R^{a} or —C(O)R^{a}. Methods for making compounds of Formula I are described, for example, in international PCT Application No. PCT/US2010/021856 (WO2010/085684), and such information is incorporated herein by reference.

The compound may also have a Formula II,

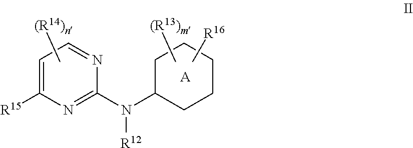

wherein

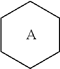

is selected from the group consisting of a six-membered aryl and a six-membered heteroaryl;

n′ is 0, 1 or 2;

m′ is 0, 1, 2, 3 or 4;

R¹² is selected from hydrogen, alkyl, alkenyl, alkynyl, haloalkyl, haloalkenyl, haloalkenyl, optionally substituted cycloalkyl, optionally substituted cycloalkylalkyl, optionally substituted cycloalkylalkenyl, optionally substituted cycloalkylalkynyl, optionally substituted aryl, optionally substituted arylalkyl, optionally substituted arylalkenyl, optionally substituted arylalkynyl, —R¹⁹—C(O)OR¹⁷, —R²⁰—N(R¹⁷)R¹⁸ and —R²⁰—OR¹⁷;

each R¹³, when present, is independently selected from the group consisting of optionally substituted alkyl, halo, haloalkyl, cyano, nitro, —OR¹⁷, —N(R¹⁷)₂, —C(O)OR¹⁷ and —C(O)N(R¹⁷)₂,

each R¹⁴, when present, is independently selected from the group consisting of alkyl, halo and haloalkyl;

R¹⁵ is selected from the group consisting of aryl and heteroaryl, where the aryl and the heteroaryl are each independently optionally substituted by one or more substituents selected from the group consisting of oxo, alkyl, halo, haloalkyl, cyano, N-heterocyclyl, N-heteroaryl, aryl, —R¹⁹—OR²¹, —R¹⁹—S(O)_{p′}R²¹ (where p′ is 0, 1 or 2), —R¹⁹—C(O)R²¹, —R¹⁹—C(O)OR²¹, —R¹⁹—C(O)N(R²¹)R²², R¹⁹—N(R²¹)R²², —R¹⁹—N(R²¹)—R²⁰—N(R²¹)R²², —R¹⁹—N(R²¹)—R²⁰—OR²², —R¹⁹—N(R²¹)C(O)R²², —R¹⁹—N(R²¹)S(O)₂R²², —R¹⁹—N(R²¹)C(O)—R¹⁹—N(R²¹)R²², and —R¹⁹—N(R²¹)—R²⁰—N(R²¹)S(O)₂R²², wherein each R²² and R²¹ independently is selected from the group consisting of hydrogen, alkyl, optionally substituted cycloalkyl, optionally substituted heterocyclyl, optionally substituted heterocyclylalkyl, optionally substituted heteroaryl and optionally substituted aryalkyl, and where the N-heterocyclyl, the N-heteroaryl and the aryl are each independently optionally substituted by one or more substituents selected from the group consisting of —C(O)R¹⁷, —R¹⁹—N(R¹⁷)R¹⁸, —R¹⁹—C(O)N(R¹⁷)R¹⁸, alkyl, halo and optionally substituted aryl,

R¹⁶ is an N-heterocyclyl, wherein a nitrogen atom in the N-heterocyclyl is optionally substituted by a substituent selected from the group consisting of alkyl, alkenyl, alkynyl, haloalkyl, haloalkenyl, haloalkynyl, optionally substituted aryl, optionally substituted aralkyl, optionally substituted arylalkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁹—OR¹⁴, —R¹⁹—C(O)R¹⁷, —R¹⁹—C(O)OR¹⁷, —R¹⁹—N(R¹⁷)R¹⁸, —R¹⁹—C(N═R¹⁷)N(R¹⁷)R¹⁸, —R¹⁹—S(O)₂N(R¹⁷)R¹⁸, and —R¹⁹—S(O)ₜR¹⁷ (where t is 1 or 2); and a carbon atom in the N-heterocyclyl is optionally substituted by a substituent selected from the group consisting of alkyl, alkenyl, alkynyl, halo, haloalkyl, haloalkenyl, haloalkynyl, oxo, optionally substituted aryl, optionally substituted aralkyl, optionally substituted arylalkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁹—OR¹⁷, —R¹⁹—C(O)R¹⁷, —R¹⁹—C(O)OR¹⁷, —R¹⁹—N(R¹⁷)R¹⁸, —R¹⁹—C(O)N(R¹⁷)R¹⁸, —R¹⁹—S(O)₂N(R¹⁷)R¹⁸, and —R¹⁹—S(O)_{p′}R¹⁷ (where p′ is 0, 1 or 2),

each R¹⁷ and each R¹⁸ is independently selected from the group consisting of hydrogen, alkyl, alkenyl, alkynyl, haloalkyl, haloalkenyl, haloalkynyl, optionally substituted cycloalkyl, optionally substituted cycloalkylalkyl, optionally substituted aryl, optionally substituted aralkyl, optionally substituted arylalkenyl, optionally substituted aralkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, and optionally substituted heteroarylalkynyl; or any R¹⁷ and R¹⁸, together with the common nitrogen to which they are both attached, form an optionally substituted N-heteroaryl or an optionally substituted N-heterocyclyl;

each R¹⁹ is independently selected from the group consisting of a direct bond, an optionally substituted straight or branched alkylene chain, an optionally substituted straight or branched alkenylene chain and an optionally substituted straight or branched alkynylene chain; and

each R²⁰ is independently selected from the group consisting of an optionally substituted straight or branched alkylene chain, an optionally substituted straight or branched alkenylene chain and an optionally substituted straight or branched alkynylene chain; provided at least one of R¹⁶ and a substituent on R¹⁵ is a bridged N-heterocyclyl. Methods for making compounds of Formula II are described, for example, in US Patent Publication Number 2009/0258864, and such information is incorporated herein by reference.

In other disclosed embodiments, the compound may have a Formula III, illustrated below.

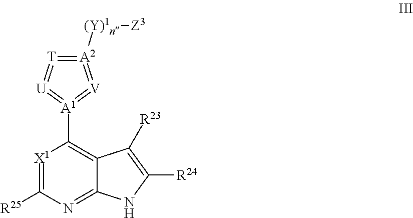

wherein A¹ and A² are independently selected from C and N;

T, U, and V are independently selected from O, S, N, CR²⁷, and NR²⁸;

the 5-membered ring formed by A¹, A², U, T, and V is aromatic;

X¹ is N or CR²⁶;

Y¹ is C₁₋₈ alkylene, C₂₋₈ alkenylene, C₂₋₈ alkynylene, (CR³³R³⁴)_{p″}—(C₃₋₁₀cycloalkylene)-(CR³³R³⁴), (CR³³R³⁴)_{p″}-(arylene)-(CR³³R³⁴)_{q}, (CR³³R³⁴)_{p″}—(C₁₋₁₀heterocycloalkylene)-(CR³³R³⁴)_{q}, (CR³³R³⁴)_{p″}-(heteroarylene)-(CR³³R³⁴)_{q}, (CR³³R³⁴)_{p″}O(CR³³R³⁴), (CR³³R³⁴)_{p″}S(CR³³R³⁴), (CR³³R³⁴)_{p″}C(O)(CR³³R³⁴), (CR³³R³⁴)_{p″}C(O)NR⁴¹(CR³³R³⁴)_{q}, (CR³³R³⁴)_{p″}C(O)O(CR³³R³⁴)_{q}, (CR³³R³⁴)_{p″}OC(O)(CCR³³R³⁴)_{q}, (CR³³R³⁴)_{p″}OC(O)NR⁴¹(CR³³R³⁴)_{q}, (CR³³R³⁴)NR⁴¹(CR³³R³⁴)_{q}, (CCR³³R³⁴)ₚNR⁴¹C(O)NR⁴²(CR³³R³⁴)_{q}, (CR³³R³⁴)_{p″}S(O)(CR³³R³⁴)_{q}, (CR³³R³⁴)_{p″}S(O)NR⁴¹(CR³³R³⁴)_{q}, (CR³³R³⁴)_{p″}S(O)₂(CR³³R³⁴)_{q}, or (CR³³R³⁴)_{p″}S(O)₂NR⁴¹(CR³³R³⁴)_{q}, wherein the C₁₋₈ alkylene, C₂₋₈ alkenylene, C₂₋₈ alkynylene, cycloalkylene, arylene, heterocycloalkylene, or heteroarylene, is optionally substituted with 1, 2, or 3 substituents independently selected from -D¹-D²-D³-D⁴;

Z³ is H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, ═C—R⁴⁹, ═N—R⁴⁹, Cy¹, CN, NO₂, OR³⁵SR³⁵, C(O)R³⁶, C(O)NR⁴¹R⁴², C(O)OR³⁵, OC(O)R³⁶, OC(O)NR⁴¹R⁴², NR⁴¹R⁴², NR⁴¹C(O)R³⁶, NR⁴¹C(O)NR⁴¹R⁴², NR⁴¹C(O)OR³⁵, C(═NR⁴⁹)NR⁴¹R⁴², NR⁴¹C(═NR⁴⁹)NR⁴¹R⁴², S(O)R³⁶, S(O)NR⁴¹R⁴², S(O)₂R³⁶, NR⁴¹S(O)₂R³⁶, C(═NOH)R³⁶, C(═NO(C₁₋₆alkyl)R³⁶, and S(O)₂NR⁴¹R⁴², wherein when Z is H, n″ is 1, or the —(Y)_{n″}—Z moiety is taken together with i) A² to which the moiety is attached, ii) R²⁷ or R²⁸ of either T or V, and iii) the C or N atom to which the R²⁷ or R²⁸ of either T or V is attached to form a 4- to 20-membered aryl, cycloalkyl, heteroaryl, or heterocycloalkyl ring fused to the 5-membered ring formed by A¹, A², U, T, and V, wherein the 4- to 20-membered aryl, cycloalkyl, heteroaryl, or heterocycloalkyl ring is optionally substituted by 1, 2, 3, 4, or 5 substituents independently selected from —(W)_{1n″}-Q;

W is C₁₋₈ alkylenyl, C₂₋₈ alkenylenyl, C₂₋₈ alkynylenyl, O, S, C(O), C(O)NR⁴³, C(O)O, OC(O), OC(O)NR⁴³, NR⁴³, NR⁴³C(O)NR⁴⁴, S(O)₂, S(O)NR⁴³, S(O)₂, or S(O)₂NR⁴³;

Q is H, halo, CN, NO₂, C₁₋₈ alkyl, C₂₋₈ alkenyl, C₂₋₈ alkynyl, C₁₋₈ haloalkyl, halosulfanyl, aryl, cycloalkyl, heteroaryl, or heterocycloalkyl, wherein the C₁₋₈ alkyl, C₂₋₈ alkenyl, C₂₋₈ alkynyl, C₁₋₈ haloalkyl, aryl, cycloalkyl, heteroaryl, or heterocycloalkyl is optionally substituted with 1, 2, 3 or 4 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy², CN, NO₂, OR³⁷, SR³⁷, C(O)R³⁸, C(O)NR⁴³R⁴⁴, C(O)OR³⁷, OC(O)R³⁸, OC(O)NR⁴³R⁴⁴, NR⁴³R⁴⁴, NR⁴³C(O)R³⁸, NR⁴³C(O)NR⁴³R⁴⁴, NR⁴³C(O)OR³⁷, S(O)R³⁸, S(O)NR⁴³R⁴⁴, S(O)₂R³⁸, NR⁴³S(O)₂R³⁸ and S(O)₂NR⁴³R⁴⁴;

Cy¹ and Cy² are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, CN, NO₂, —OR³⁹, SR³⁹, C(O)R⁴⁰, C(O)NR⁴⁵R⁴⁶, C(O)OR³⁹, OC(O)R⁴⁰, OC(O)NR⁴⁵R⁴⁶, NR⁴⁵R⁴⁶, NR⁴⁵C(O)R⁴⁰, NR⁴⁵C(O)OR³⁹, NR⁴⁵S(O)R⁴⁰, NR⁴⁵S(O)₂R⁴⁰, S(O)R⁴⁰, S(O)NR⁴⁵R⁴⁶, S(O)₂R⁴⁰, and S(O)₂NR⁴⁵R⁴⁶;

R²³, R²⁴, R²⁵, and R²⁶ are independently selected from H, halo, C₁₋₄ alkyl, C₂₋₄ alkenyl, C₂₋₄ alkynyl, C₁₋₄ haloalkyl, halosulfanyl, aryl, cycloalkyl, heteroaryl, heterocycloalkyl, CN, NO₂, OR²⁹, SR²⁹, C(O)R³⁰, C(O)NR³¹R³², C(O)OR²⁹OC(O)R³⁰, OC(O)NR³¹R³², NR³¹R³², NR³¹C(O)R³⁰, NR³²C(O)OR²⁹, S(O)R³⁰, S(O)NR³¹R³², S(O)₂R³⁰, NR³¹S(O)₂R³⁰, and S(O)₂NR³¹R³²;

R²⁷ is H, halo, C₁₋₄ alkyl, C₂₋₄ alkenyl, C₂₋₄ alkynyl, C₁₋₄ haloalkyl, halosulfanyl, CN, NO₂, OR²⁹, SR²⁹, C(O)R³⁰, C(O)NR³¹R³², C(O)OR²⁹, OC(O)R³⁰, OC(O)NR³¹R³², NR³¹R³², NR³¹C(O)R³⁰, NR³¹C(O)OR²⁹, S(O)R³⁰, S(O)NR³¹R³², S(O)₂R³⁰, NR³¹S(O)₂R³⁰, or S(O)₂NR³¹R³²;

R²⁸ is H, C₁₋₄ alkyl, C₂₋₄ alkenyl, C₂₋₄ alkynyl, C₁₋₄ haloalkyl, OR²⁹, C(O)R³⁰, C(O)NR³¹R³², C(O)OR²⁹, S(O)R³⁰, S(O)NR³¹R³², S(O)₂R³⁰, or S(O)₂NR³¹R³²;

R²⁹ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;

R³⁰ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;

R³¹ and R³² are independently selected from H, C₁₋₁₀ alkyl, C₁₋₆ haloalkyl, C₂₋₆ alkenyl, C₂₋₆ alkynyl, C₁₋₆ alkylcarbonyl, arylcarbonyl, C₁₋₆ alkylsulfonyl, arylsulfonyl, aryl, heteroaryl, cycloalkyl, heterocycloalkyl, arylalkyl, heteroarylalkyl, cycloalkylalkyl and heterocycloalkylalkyl;

or R³¹ and R³² together with the N atom to which they are attached form a 4-, 5-, 6- or 7-membered heterocycloalkyl group;

R³³ and R³⁴ are independently selected from H and -E¹-E²-E³-E⁴;

D¹ and E¹ are independently absent or independently selected from absent or selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆alkyl, C₁₋₆haloalkyl, C₂₋₈ alkoxyalkyl, C₁₋₆ alkoxy, C₁₋₆haloalkoxy, amino, C₁₋₆ alkylamino, and C₂₋₈ dialkylamino;

D² and E² are independently absent or independently selected from C₁₋₆ alkylene, C₂₋₆ alkenylene, C₂₋₆ alkynylene, (C₁₋₆alkylene)ᵣ-O—C₁₋₆alkylene)ₛ, (C₁₋₆alkylene)ᵣ-S—(C₁₋₆alkylene)ₛ, (C₁₋₆ alkylene)ᵣ-NR⁴⁷—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR⁴⁷—(C₁₋₆ alkylene)ₛ, (C₁₋₆ alkylene), —SO—(C₁₋₆ alkylene)ₛ, (C₁₋₆ alkylene)ᵣ-SO₂—(C₁₋₆ alkylene)ₛ, (C₁₋₆ alkylene)ᵣ-SONR⁴¹—(C₁₋₆alkylene)ₛ, and (C₁₋₆alkylene)ᵣ-NR⁴⁷CONR⁴⁸—(C₁₋₆ alkylene)ₛ, wherein each of the C₁₋₆ alkylene, C₂₋₆ alkenylene, and C₂₋₆ alkynylene is optionally substituted by 1, 2 or 3 substituents independently selected from halo, CN, NO₂, N₃, SCN, OH, C₁₋₆ alkyl, C₁₋₆ haloalkyl, C₂₋₈ alkoxyalkyl, Ĉalkoxy, C₁₋₆ haloalkoxy, amino, C₁₋₆ alkylamino, and C₂₋₈ dialkylamino;

D³ and E³ are independently absent or independently selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆ alkyl, C₁₋₆haloalkyl, C₂₋₈ alkoxyalkyl, C₁₋₆ alkoxy, C₁₋₆ haloalkoxy, amino, C₁₋₆ alkylamino, and C₂₋₈ dialkylamino;

D⁴ and E⁴ are independently selected from H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy¹, CN, NO₂, OR³⁵, SR³⁵, C(O)R³⁶, C(O)NR⁴¹R⁴², C(O)OR³⁵, OC(O)R³⁶, OC(O)NR⁴¹R⁴², NR⁴¹R⁴², NR⁴¹C(O)R³⁶, NR⁴¹C(O)NR⁴¹R⁴², NR⁴¹C(O)OR³⁵, C(═NR⁴⁹)NR⁴¹R⁴², NR⁴¹C(═NR⁴⁹)NR⁴¹R⁴², S(O)R³⁶, S(O)NR⁴¹R⁴², S(O)₂R³⁶, NR⁴¹S(O)₂R³⁶, C(═NOH)R³⁶, C(═NO(C₁₋₆ alkyl)R³⁶, and S(O)₂NR⁴¹R⁴², wherein the C₁₋₈ alkyl, C₂₋₈ alkenyl, or C₂₋₈ alkynyl, is optionally substituted with 1, 2, 3, 4, 5, or 6 substituents independently selected from halo, C₁₋₄ alkyl, C₂₋₄ alkenyl, C₂₋₄ alkynyl, C₁₋₄haloalkyl, halosulfanyl, C₁₋₄hydroxyalkyl, C₁₋₄cyanoalkyl, Cy¹, CN, NO₂, OR³⁵, SR³⁵, C(O)R³⁶. C(O)N42, R⁴¹R⁴², C(O)OR³⁵, OC(O)R³⁶, OC(O)NR⁴¹R⁴², NR⁴¹R⁴², NR⁴¹C(O)R³⁶, NR⁴¹C(O)NR⁴¹R⁴², NR⁴¹C(O)OR³⁵, C(═NR⁴⁹)NR⁴¹R⁴², NR^{c}C(═NR⁴⁹)NR⁴¹R⁴², S(O)R³⁶, S(O)NR⁴¹R⁴², S(O)₂R²⁸, NR⁴¹S(O)₂R³⁶, C(═NOH)R³⁶, C(═NO(C₁₋₆alkyl))R³⁶, and S(O)₂NR⁴¹R⁴²;

R³⁵ is Cy¹, —(C₁₋₆alkyl)-Cy¹, C₁₋₆ alkyl, C₁₋₆ haloalkyl, C₂₋₆ alkenyl, C₂₋₆ alkynyl, wherein the C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;

R³⁶ is H, Cy¹, —(C₁₋₆ alkyl)-Cy¹, C₁₋₆ alkyl, C₁₋₆ haloalkyl, C₂₋₆ alkenyl, C₂₋₆ alkynyl, wherein the C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆alkyl, C₁₋₆ haloalkyl, C₁₋₆ haloalkyl, halosulfanyl, aryl, arylalkyl, heteroaryl, heteroarylalkyl, cycloalkyl and heterocycloalkyl;

R³⁷ and R³⁹ are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein the C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;

R³⁸ and R⁴⁰ are independently selected from H, C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein the C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆haloalkyl, halosulfanyl, aryl, arylalkyl, heteroaryl, heteroarylalkyl, cycloalkyl and heterocycloalkyl;

R⁴¹ and R⁴² are independently selected from H, Cy¹, —(C₁₋₆alkyl)-Cy¹, C₁₋₁₀ alkyl, C₁₋₆haloalkyl, C₂₋₆ alkenyl, C₂₋₆ alkynyl, wherein the C₁₋₁₀ alkyl, C₁₋₆ haloalkyl, C₂₋₆ alkenyl, or C₂₋₆ alkynyl, is optionally substituted with 1, 2, or 3 substituents independently selected from Cy¹, —(C₁₋₆alkyl)-Cy¹, OH, CN, amino, halo, C₁₋₆alkyl, C₁₋₆haloalkyl, C₁₋₆haloalkyl, and halosulfanyl; or

R⁴¹ and R⁴² together with the N atom to which they are attached form a 4-, 5-, 6- or 7-membered heterocycloalkyl group optionally substituted with 1, 2, or 3 substituents independently selected from Cy¹, —(C₁₋₆ alkyl)-Cy¹, OH, CN, amino, halo, C₁₋₆alkyl, C₁₋₆haloalkyl, C₁₋₆haloalkyl, and halosulfanyl;

R⁴³ and R⁴⁴ are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein the C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl; or

R⁴³ and R⁴⁴ together with the N atom to which they are attached form a 4-, 5-, 6- or 7-membered heterocycloalkyl group optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;

R⁴⁵ and R⁴⁶ are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein the C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl; or

R⁴⁵ and R⁴⁶ together with the N atom to which they are attached form a 4-, 5-, 6- or 7-membered heterocycloalkyl group optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;

R⁴⁷ and R⁴⁸ are independently selected from H and C₁₋₆alkyl;

R⁴⁹ is H, CN, C₁₋₆ alkyl or NO₂;

m″ is 0 or 1,

n″ is 0 or 1,

p″ is 0, 1, 2, 3, 4, 5, or 6,

q is 0, 1, 2, 3, 4, 5, or 6,

r is 0 or 1,

s is 0 or 1.

Methods for making compounds of Formula III are described, for example, in international patent publication numbers WO2010/039939, WO2007/070514, and WO2009/114512, and such information is incorporated herein by reference.

Additionally, the compound may have a Formula IV, illustrated below

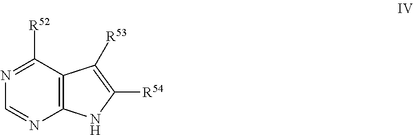

wherein R⁵² is a group of the formula

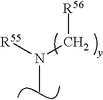

wherein y is 0, 1 or 2 or heteroaryl;

R⁵⁵ is selected from the group consisting of hydrogen, (C₁-C₆)alkyl, (C₁-C₆)alkylsulfonyl, (C₂-C₆)alkenyl, (C₂-C₆)alkynyl wherein the alkyl, alkenyl and alkynyl groups are optionally substituted by deuterium, hydroxy, amino, trifluoromethyl, (C₁-C₄)alkoxy, (C₁-C₆)acyloxy, (C₁-C₆)alkylamino, ((C₁-C₆)alkyl)₂amino, cyano, nitro, (C₂-C₆)alkenyl, (C₂-C₆)alkynyl or (C₁-C₆)acylamino, or R⁵⁵ is (C₃-C₁₀)cycloalkyl wherein the cycloalkyl group is optionally substituted by deuterium, hydroxy, amino, trifluoromethyl, (C₁-C₆)acyloxy, (C₁-C₆)acylamino, (C₁-C₆)alkylamino, ((C₁-C₆)alkyl)₂amino, cyano, cyano(C₁-C₆)alkyl, trifluoromethyl(C₁-C₆)alkyl, nitro, nitro(CC₆)alkyl or (C₁-C₆)acylamino;

R⁵⁶ is (C₂-C₉)heterocycloalkyl wherein the heterocycloalkyl groups must be substituted by one to five carboxy, cyano, amino, deuterium, hydroxy, (C₁-C₆)alkyl, (C₁-C₆)alkoxy, halo, (C₁-C₆)acyl, (C₁-C₆alkylamino), amino(C₁-C₆)alkyl, (C₁-C₆)alkoxy-CO—NH, (C₁-C₆)alkylamino-CO—, (C₂-C₆)alkenyl, (C₂-C₆)alkynyl, (C₁-C₆)alkylamino, amino(C₁-C₆)alkyl, hydroxy(C₁-C₆)alkyl, (C₁-C₆)alkoxy(C₁-C₆)alkyl, (C₁-C₆)acyloxy(C₁-C₆)alkyl, nitro, cyano(C₁-C₆)alkyl, halo(C₁-C₆)alkyl, nitro(C₁-C₆)alkyl, trifluoromethyl, trifluoromethyl(C₁-C₆)alkyl, (C₁-C₆)acylamino, (C₁-C₆)acylamino(C₁-C₆)alkyl, (C₁-C₆)alkoxy(C₁-C₆)acylamino, amino(C₁-C₆)acyl, amino(C₁-C₆)acyl(C₁-C₆)alkyl, (C₁-C₆)alkylamino(C₁-C₆)acyl, ((C₁-C₆)alkyl)₂amino(C₁-C₆)acyl, R⁶⁴R⁶⁵N—CO—O—, R⁶⁴R⁶⁵N—CO—(C₁-C₆)alkyl, (C₁-C₆)alkyl-S(O)_{m′″}, R⁶⁴R⁶⁵NS(O)_{m′″}, R⁶⁴R⁶⁵NS(O)_{m′″}(C₁-C₆)alkyl, R⁶⁴S(O)_{m′″}R⁶⁵N, R⁶⁴S(O)_{m′″}R⁶⁵N(C₁-C₆)alkyl wherein m is 0, 1 or 2 and R⁶⁴ and R⁶⁵ are each independently selected from hydrogen or a group of the formula

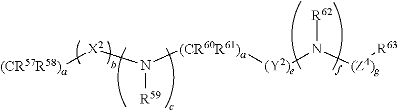

wherein a is 0, 1, 2, 3 or 4;

b, c, e, f and g are each independently 0 or 1, d is 0, 1, 2, or 3;

X² is S(O)_{n′″} wherein n is 0, 1 or 2, oxygen, carbonyl or —C(═N-cyano)-;

Y² is S(O)_{n′″} wherein n is 0, 1 or 2; or carbonyl; and

Z⁴ is carbonyl, C(O)O—, C(O)NR— or S(O)_{n′″} wherein n′″ is 0, 1 or 2;

R⁵⁷, R⁵⁸, R⁵⁹, R⁶⁰, R⁶¹ and R⁶² are each independently selected from the group consisting of hydrogen or (C₁-C₆)alkyl optionally substituted by deuterium, hydroxy, amino, trifluoromethyl, (C₁-C₆)acyloxy, (C₁-C₆)acylamino, ((C₁-C₆)alkyl)₂amino, cyano, cyano(C₁-C₆)alkyl, trifluoromethyl(C₁-C₆)alkyl, nitro, nitro(C₁-C₆)alkyl, or (C₁-C₆)acylamino,

R⁶³ is carboxy, cyano, amino, oxo, deuterium, hydroxy, trifluoromethyl, (C₁-C₆)alkyl, trifluoromethy C₁-C₆alkyl, (C₁-C₆)alkoxy, halo, (C₁-C₆)acyl, (C₁-C₆)alkylamino, ((C₁-C₆)alkyl)₂ amino, amino(C₁-C₆)alkyl, (C₁-C₆)alkoxy-CO—NH—, (C₁-C₆)alkylamino-CO—, (C₂-C₆)alkenyl, (C₂-C₆) alkynyl, (C₁-C₆)alkylamino, hydroxy(C₁-C₆)alkyl, (C₁-C₆)alkoxy(C₁-C₆)alkyl, (C₁-C₆)acyloxy(C₁-C₆)alkyl, nitro, cyano(C₁-C₆)alkyl, halo(C₁-C₆)alkyl, nitro(C₁-C₆)alkyl, trifluoromethyl, trifluoromethyl(C₁-C₆)alkyl, (C₁-C₆)acylamino, (C₁-C₆)acylamino(C₁-C₆)alkyl, (C₁-C₆)alkoxy(C₁-C₆)acylamino, amino(C₁-C₆)acyl, amino(C₁-C₆)acyl(C₁-C₆)alkyl, (C₁-C₆alkylamino(C₁-C₆)acyl, ((C₁-C₆)alkyl)₂amino(C₁-C₆)acyl, R⁶⁴R⁶⁵N—CO—O—, R⁶⁴R⁶⁵N—CO—(C₁-C₆)alkyl, R⁶⁴C(O)NH, R⁶⁴OC(O)NH, R⁶⁴NHC(O)NH, (C₁-C₆)alkyl-S(O)_{m′″}, (C₁-C₆)alkyl-S(O)_{m′″}—(C₁-C₆)alkyl, R⁶⁴R⁶⁵NS(O)_{m′″}, R⁶⁴R⁶⁵NS(O)_{m′″}(C₁-C₆alkyl, R⁶⁴S(O)_{m′″}R⁶⁵N, R⁶⁴S(O)_{m′″}R⁶⁵N(C₁-C₆)alkyl wherein m is 0, 1 or 2 and R⁶⁴ and R⁶⁵ are each independently selected from hydrogen or (C₁-C₆)alkyl;

R⁵³ and R⁵⁴ are each independently selected from the group consisting of hydrogen, deuterium, amino, halo, hydroxy, nitro, carboxy, (C₂-C₆)alkenyl, (C₂-C₆)alkynyl, trifluoromethyl, trifluoromethoxy, (C₁-C₆)alkyl, (C₁-C₆alkoxy, (C₃-C₁₀)cycloalkyl wherein the alkyl, alkoxy or cycloalkyl groups are optionally substituted by one to three groups selected from halo, hydroxy, carboxy, amino (C₁-C₆)alkylthio, (C₁-C₆alkylamino, ((C₁-C₆)alkyl)₂amino, (C₅-C₉)heteroaryl, (C₂-C₉)heterocycloalkyl, (C₃-C₉)cycloalkyl or (C₆-C₁₀)aryl, or R⁵³ and R⁵⁴ are each independently (C₃-C₁₀)cycloalkyl, (C₃-C₁₀)cycloalkoxy, (C₁-C₆)alkylamino ((C₁-C₆)alkyl)₂amino, (C₆-C₁₀)arylamino, (C₁-C₆)alkylthio, (C₆-C₁₀)arylthio, (C₁-C₆)alkylsulfinyl, (C₆-C₁₀)arylsulfinyl, (C₁-C₆)alkylsulfonyl, (C₆-C₁₀)arylsulfonyl, (C₁-C₆)acyl, (C₁-C₆alkoxy-CO—NH—, (C₁-C₆)alkyamino-CO—, (C₅-C₉)heteroaryl, (C₂-C₉)heterocycloalkyl or (C₆-C₁₀)aryl wherein the heteroaryl, heterocycloalkyl and aryl groups are optionally substituted by one to three halo, (C₁-C₆)alkyl, (C₁-C₆)alkyl-CO—NH—, (C₁-C₆)alkyoxy-CO—NH—, (C₁-C₆)alkyl-CO—NH—(C₁-C₆)alkyl, (C₁-C₆)alkoxy-CO—NH—(C₁-C₆)alkyl, (C₁-C₆alkoxy-CO—NH—(C₁-C₆)alkoxy, carboxy, carboxy(C₁-C₆)alkyl, carboxy(C₁-C₆)alkoxy, benzyloxycarbonyl(C₁-C₆)alkoxy, (C₁-C₆alkoxycarbonyl(C₁-C₆)alkoxy, (C₁₋₆—C₁₋₁₀)aryl, amino, amino(C₁-C₆)alkyl, (C₁-C₆)alkoxycarbonylamino, (C₆-C₁₀)aryl(C₁-C₆)alkoxycarbonylamino, (C₁-C₆)alkylamino, ((C₁-C₆)alkyl)₂amino, (C₁-C₆)alkylamino(C₁-C₆)alkyl, ((C₁-C₆)alkyl)₂amino(C₁-C₆)alkyl, hydroxy, (C₁-C₆alkoxy, carboxy, carboxy(C₁-C₆)alkyl, (C₁-C₆alkoxycarbonyl, (C₁-C₆alkoxycarbony C₁-C₆alkyl, (C₁-C₆)alkoxy-CO—NH—, (C₁-C₆)alkyl-CO—NH—, cyano, (C₅-C₉)heterocycloalkyl, amino-CO—NH—, (C₁-C₆)alkylamino-CO—NH—, ((C₁-C₆)alkyl)₂amino-CO—NH—, (C₆-C₁₀)arylamino-CO—NH—, (C₅-C₉)heteroarylamino-CO—NH—, (C₁-C₆)alkylamino-CO—NH—(C₁-C₆)alkyl, ((C₁-C₆)alkyl)₂amino-CO—NH—(C₁-C₆)alkyl, (C₆-C₁₀)arylamino-CO—NH—(C₁-C₆)alkyl, (C₅-C₉)heteroarylamino-CO—NH—(C₁-C₆)alkyl, (C₁-C₆)alkylsulfonyl, (C₁-C₆)alkylsulfonylamino, (C₁-C₆)alkylsulfonylamino(C₁-C₆)alkyl, (C₆-C₁₀)arylsulfonyl, (C₆-C₁₀)arylsulfonylamino, (C₆-C₁₀)arylsulfonylamino(C₁-C₆)alkyl, (C₁-C₆)alkylsulfonylamino, (C₁-C₆)alkylsulfonylamino(C₁-C₆)alkyl, (C₅-C₉)heteroaryl or (C₂-C₉)heterocycloalkyl.

Methods for making compounds of Formula IV are described, for example, in international patent publication number WO2001/42246, and such information is incorporated herein by reference.

In other disclosed embodiments, the compound is a compound, having a formula V,

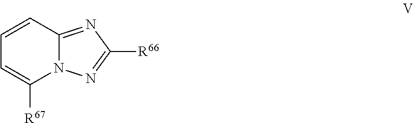

wherein R⁶⁶ is an amine or an amide and R⁶⁷ is selected from optionally substituted aryl or heteroaryl. Methods for making compounds of formula V are described, for example, in U.S. Pat. No. 8,088,764, and such information is incorporated herein by reference.

Other disclosed embodiments concern compounds having a general formula VI

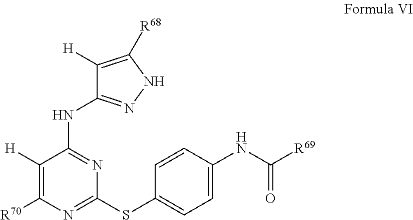

wherein R⁶⁸ is C₁₋₃alkyl or cyclopropyl, R⁶⁹ is CH₂CH₃, CH₂CF₃, CH₂CH₂CF₃,

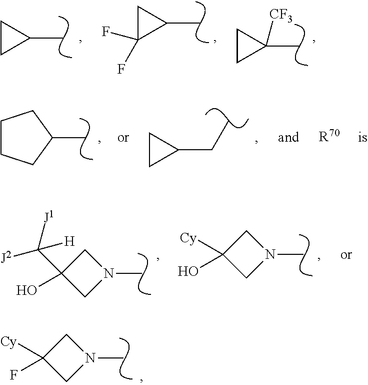

wherein J¹ is H or CH₃, J² is C₁₋₄alkyl, and Cy is C₃₋₅cycloalkyl. Methods for making compounds of formula VI are described, for example, in U.S. Pat. No. 8,129,399, and such information is incorporated herein by reference.

In other disclosed embodiments, the compound may have a general formula VII

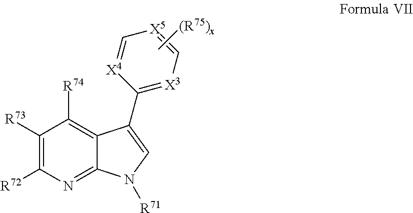

or pharmaceutically acceptable salts thereof, wherein R⁷¹ is T₁-R′ or is —Si(R′)₃;

R⁷², R⁷³, and R⁷⁴ are each independently halogen, CN, NO₂, or V¹—R′;

X³, X⁴ and X⁵ are each independently N, or CH, wherein the hydrogen atom of CH is optionally replaced by R⁷⁵;

x is 1, 2, 3, or 4;

each occurrence of R⁷⁵ is independently halogen, CN, NO₂, or U¹—R′;

T¹, V¹, and U¹ are each independently a bond or an optionally substituted C₁-C₆ alkylidene chain, wherein up to two methylene units of the chain are optionally and independently replaced by —NR′—, —S—, —O—, —CS—, —CO₂—, —OCO—, —CO—, —COCO—, —CONR′—, —NR′CO—, —NR′CO₂—, —SO₂NR′—, —NR′SO₂—, —CONR′NR′—, —NR′CONR′—, —OCONR′—, —NR′NR′—, —NR′SO₂NR′—, —SO—, —SO₂—, —PO—, —PO₂—, or —POR′—;

and each occurrence of R′ is independently hydrogen or an optionally substituted group selected from a C₁-C₆ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or two occurrences of R′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur. In certain embodiments, the two occurrences of R′ that form a ring will be on a single substituent (e.g., R⁷¹, R⁷², R⁷³, R⁷⁴ or on a single R⁷⁵ substituent) and form a monocyclic or bicyclic ring. In other embodiments, the two occurrences of R′ are on two substituents (e.g., on two R⁷⁵ substituents) and can form a bicyclic fused ring with the ring to which the R⁷⁵ substituents are attached. However, the two occurrences of R′ do not form a tricyclic ring whether they are a bound to a single substituent or to two separate substituents.

In particular disclosed embodiments, it is further provided that if R⁷¹ is substituted cyclopentyl, x is 1, X³ and X⁵ are CH, then X⁴ is not C—R⁷⁵, where R⁷⁵ is fluoro or OMe;

if R⁷² and R⁷³ are simultaneously H and R⁷¹ and R⁷⁴ are independently selected from H or Me, x is 1, X³ and X⁵ are CH, then X⁴ is not C—R⁷⁵, where R⁷⁵ is OMe, NO₂, or fluoro;

if R⁷¹, R⁷² and R⁷⁴ are simultaneously H, x is 1, R⁷⁵ is —SMe, NH₂ or an optionally substituted NH-piperidine, and X³ and X⁴ are N, then X⁵ is not CH;

if R⁷², R⁷³ and R⁷⁴ are simultaneously H, X³, X⁴ and X⁵ are CH, and two R⁷⁵ form a fused optionally substituted bicyclic ring with the ring to which they are attached, then R⁷¹ is not CH₂CH₂N(Me)₂;

if R⁷² and R⁷³ are simultaneously H, R⁷⁴ is NH₂, and X³, X⁴ and X⁵ are CH, then R⁷¹ is not substituted phenyl;

if R⁷², R⁷³ and R⁷⁴ are simultaneously H, then R⁷¹ is not Si(R′)₃;

if R⁷¹, R⁷² and R⁷⁴ are simultaneously H and (i) X⁴ and X⁵ are CH or CR⁷⁵ or (ii) any one of X³, X⁴ or X⁵ are N, then R⁵ is not phenyl or phenyl substituted with O-phenyl or N(Me)₂.

Methods for making compounds of formula VII are described, for example, in U.S. Pat. No. 8,188,281, and such information is incorporated herein by reference.

In other disclosed embodiments, the compound may have a formula VIII

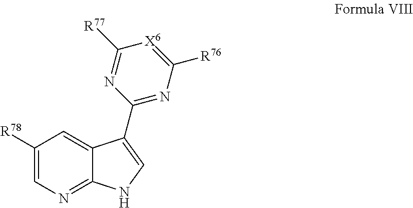

or a pharmaceutically acceptable salt thereof, wherein R⁷⁸ is H, Cl or F;

X⁶ is N or CR⁷⁹;

R⁷⁷ is H, F, R′, OH, OR′, COR′, COOH, COOR′, CONH₂, CONHR′, CON(R′)₂, or CN;

R⁷⁹ is H, F, R′, OH, OR′, COR′, COOH, COOR′, CONH₂, CONHR′, CON(R′)₂, or CN;

or R⁷⁷ and R⁷⁹, taken together, form a 5-7 membered aryl or heteroaryl ring optionally substituted with 1-4 occurrences of R⁸⁰;

R′ is a C₁₋₃aliphatic optionally substituted with 1-4 occurrences of R⁸¹;

each R⁸¹ is independently selected from halogen, CF₃, OCH₃, OH, SH, NO₂, NH₂, SCH₃, NCH₃, CN or unsubstituted C₁₋₂ aliphatic, or two R⁸¹ groups, together with the carbon to which they are attached, form a cyclopropyl ring or C═O;

each R⁸⁰ is independently selected from halogen, OCH₃, OH, NO₂, NH₂, SH, SCH₃, NCH₃, CN or unsubstituted C₁₋₂aliphatic;

R⁷⁶ is

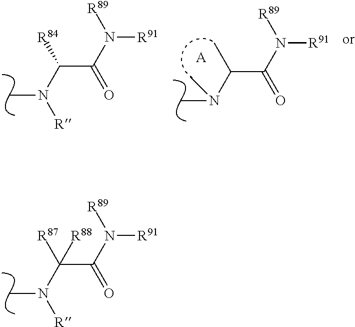

R″ is H or is a —C₁₋₂ aliphatic optionally substituted with 1-3 occurrences of R⁸²;

each R⁸² is independently selected from halogen, OCH₃, OH, SH, NO₂, NH₂, SCH₃, NCH₃, CN, CON(R⁸³)₂, or unsubstituted C₁₋₂ aliphatic, or two R⁸² groups, together with the carbon to which they are attached, form a cyclopropyl ring or C═O;

R⁸⁴ is a C₁₋₄ aliphatic optionally substituted with 1-5 occurrences of R⁸⁵;

each R⁸⁵ is independently selected from halogen, OCH₃, OH, NO₂, NH₂, SH, SCH₃, NCH₃, CN or unsubstituted C₁₋₂aliphatic, or two R⁸⁵ groups, together with the carbon to which they are attached, form a cyclopropyl ring;

Ring A is a 4-8 membered saturated nitrogen-containing ring comprising up to two additional heteroatoms selected from N, O, or S and optionally substituted with 1-4 occurrences of R⁸⁶;

each R⁸⁶ is independently selected from halogen, R′, NH₂, NHR′, N(R′)₂, SH, SR′, OH, OR′, NO₂, CN, CF₃, COOR′, COOH, COR′, OC(O)H, OC(O)R′, CONH₂, CONHR′, CON(R′)₂, NHC(O)R′ or NR′C(O)R; or any two R⁸⁶ groups, on the same substituent or different substituents, together with the atom(s) to which each R⁸⁶ group is bound, form a 3-7 membered saturated, unsaturated, or partially saturated carbocyclic or heterocyclic ring optionally substituted with 1-3 occurrences of R⁸¹;

R⁸⁷ is C₁₋₄ aliphatic optionally substituted with 1-5 occurrences of R⁸⁵;

R⁸⁸ is C₁₋₂ alkyl; or

R⁸⁷ and R⁸⁸ are taken together to form a 3-7 membered carbocyclic or heterocyclic saturated ring optionally substituted with 1-5 occurrences of R⁸⁵;

R⁸⁹ is H or unsubstituted C₁₋₂ alkyl;

R⁹⁰ is H or unsubstituted C₁₋₂ alkyl; and

R⁹¹ is a C₂₋₃ aliphatic or cycloaliphatic optionally substituted with up to 6 occurrences of F.

Methods for making compounds of formula VIII are described, for example, in U.S. Pat. No. 8,163,917, and such information is incorporated herein by reference.

In certain disclosed embodiments, the one or more compounds may be a pyrimidine-containing compound having either formula I or II, wherein the compound is selected from any one of the following compounds provided in Table I.

TABLE I
5-(2-(3-isobutoxy-5-(trifluoromethyl)phenylamino)-5-methylpyrimidin-4-
ylamino)benzo[d]oxazol-2(3H)-one;
(5-(2-(4-(cyclobutylcarbamoyl)phenylamino)-5-methylpyrimidin-4-ylamino)-2-
oxobenzo[d]oxazol-3(2H)-yl)methyl dihydrogen phosphate;
(5-(2-(4-carbamoylphenylamino)-5-methylpyrimidin-4-ylamino)-2-oxobenzo[d]oxazol-
3(2H)-yl)methyl dihydrogen phosphate;
(5-(2-(4-fluoro-3-methoxy-5-methylphenylamino)-5-methylpyrimidin-4-ylamino)-2-
oxobenzo[d]oxazol-3(2H)-yl)methyl dihydrogen phosphate calcium salt;
(5-(2-(4-fluoro-3-methoxy-5-methylphenylamino)-5-methylpyrimidin-4-ylamino)-2-
oxobenzo[d]oxazol-3(2H)-yl)methyl dihydrogen phosphate;
(R)-5-(2-(6-((1-benzylpiperidin-3-yl)(methyl)amino)pyridin-3-ylamino)-5-
methylpyrimidin-4-ylamino)benzo[d]oxazol-2(3H)-one;
(R)-5-(2-(6-(3-(dimethylamino)pyrrolidin-1-yl)pyridin-3-ylamino)-5-methylpyrimidin-4-
ylamino)benzo[d]oxazol-2(3H)-one;
(R)-5-(2-(6-(3,4-dimethylpiperazin-1-yl)pyridin-3-ylamino)-5-methylpyrimidin-4-
ylamino)benzo[d]oxazol-2(3H)-one;
(R)-5-(2-(6-(4-(cyclopropylmethyl)-3-methylpiperazin-1-yl)pyridin-3-ylamino)-5-
methylpyrimidin-4-ylamino)benzo[d]oxazol-2(3H)-one;
(R)-5-(2-(6-(4-isopropyl-3-methylpiperazin-1-yl)pyridin-3-ylamino)-5-methylpyrimidin-4-
ylamino)benzo[d]oxazol-2(3H)-one;
(R)-5-(5-methyl-2-(6-(3-(methylamino)piperidin-1-yl)pyridin-3-ylamino)pyrimidin-4-
ylamino)benzo[d]oxazol-2(3H)-one;
(R)-5-(5-methyl-2-(6-(3-methyl-4-(2,2,2-trifluoroacetyl)piperazin-1-yl)pyridin-3-
ylamino)pyrimidin-4-ylamino)benzo[d]oxazol-2(3H)-one;
(R)-5-(5-methyl-2-(6-(methyl(piperidin-3-yl)amino)pyridin-3-ylamino)pyrimidin-4-
ylamino)benzo[d]oxazol-2(3H)-one;
(R)-5-(5-methyl-2-(6-(piperidin-3-ylamino)pyridin-3-ylamino)pyrimidin-4-
ylamino)benzo[d]oxazol-2(3H)-one;
(R)-diethyl2-methyl-4-(5-(5-methyl-4-(2-oxo-2,3-dihydrobenzo[d]oxazol-5-
ylamino)pyrimidin-2-ylamino)pyridin-2-yl)piperazin-1-ylphosphonate;
(R)-tert-butyl methyl(1-(5-(5-methyl-4-(2-oxo-2,3-dihydrobenzo[d]oxazol-5-
ylamino)pyrimidin-2-ylamino)pyridin-2-yl)piperidin-3-yl)carbamate;
(S)-2-Methyl-4-{5-[5-methyl-4-(2-oxo-2,3-dihydro-benzooxazol-5-ylamino)-pyrimidin-2-
ylamino]-pyridin-2-yl}-piperazine-1-carboxylic acid tert-butyl ester;
(S)-5-(2-(6-((1-benzylpiperidin-3-yl)(methyl)amino)pyridin-3-ylamino)-5-methylpyrimidin-
4-ylamino)benzo[d]oxazol-2(3H)-one;
(S)-5-(2-(6-(1-benzylpiperidin-3-ylamino)pyridin-3-ylamino)-5-methylpyrimidin-4-
ylamino)benzo[d]oxazol-2(3H)-one;
(S)-5-(2-(6-(3-(dimethylamino)pyrrolidin-1-yl)pyridin-3-ylamino)-5-methylpyrimidin-4-
ylamino)benzo[d]oxazol-2(3H)-one;
(S)-5-(5-methyl-2-(6-(methyl(piperidin-3-yl)amino)pyridin-3-ylamino)pyrimidin-4-
ylamino)benzo[d]oxazol-2(3H)-one;
(S)-5-(5-methyl-2-(6-(piperidin-3-ylamino)pyridin-3-ylamino)pyrimidin-4-
ylamino)benzo[d]oxazol-2(3H)-one;
(S)-tert-butyl methyl(1-(5-(5-methyl-4-(2-oxo-2,3-dihydrobenzo[d]oxazol-5-
ylamino)pyrimidin-2-ylamino)pyridin-2-yl)piperidin-3-yl)carbamate;
(Z)-2-Methyl-9-[5-methyl-4-(2-oxo-2,3-dihydro-benzooxazol-5-ylamino)-pyrimidin-2-
ylamino]-3,6-dihydro-2H-benzo[c]azocin-1-one;
(Z)-5-(5-fluoro-2-(2-methyl-1-oxo-1,2,3,6-tetrahydrobenzo[c]azocin-9-ylamino)pyrimidin-
4-ylamino)benzo[d]oxazol-2(3H)-one formate salt;
(Z)-5-(5-methyl-2-(1-oxo-2,3-dihydro-1H-benzo[c]azepin-7-ylamino)pyrimidin-4-
ylamino)benzo[d]oxazol-2(3H)-one;
(Z)-5-(5-methyl-2-(2-methyl-1-oxo-2,3-dihydro-1H-benzo[c]azepin-7-ylamino)pyrimidin-
4-ylamino)benzo[d]oxazol-2(3H)-one;
{4-[5-Methyl-4-(2-oxo-2,3-dihydro-benzooxazol-5-ylamino)-pyrimidin-2-ylamino]-
phenyl}-acetaldehyde;
1-benzyl-3-(1-(5-(5-methyl-4-(2-oxo-2,3-dihydrobenzo[d]oxazol-5-ylamino)pyrimidin-2-
ylamino)pyridin-2-yl)pyrrolidin-3-yl)urea;
1-ethyl-3-(1-(5-(5-methyl-4-(2-oxo-2,3-dihydrobenzo[d]oxazol-5-ylamino)pyrimidin-2-
ylamino)pyridin-2-yl)pyrrolidin-3-yl)urea;
1-tert-butyl-3-(1-(5-(5-methyl-4-(2-oxo-2,3-dihydrobenzo[d]oxazol-5-ylamino)pyrimidin-2-
ylamino)pyridin-2-yl)pyrrolidin-3-yl)urea;
2-(2,4-Difluoro-3-methoxy-phenylamino)-4-(2-oxo-2,3-dihydro-benzooxazol-5-ylamino)-
pyrimidine-5-carboxylic acid methyl ester trifluoroacetate salt;
2-(3-Dimethylamino-4-methyl-phenylamino)-4-(2-oxo-2,3-dihydro-benzooxazol-5-
ylamino)-pyrimidine-5-carboxylic acid methyl ester trifluoroacetate salt;
2-(3-Methoxy-4,5-dimethyl-phenylamino)-4-(2-oxo-2,3-dihydro-benzooxazol-5-ylamino)-
pyrimidine-5-carboxylic acid methyl ester trifluoroacetate salt;
2-(3-Methoxy-4-methyl-phenylamino)-4-(2-oxo-2,3-dihydro-benzooxazol-5-ylamino)-
pyrimidine-5-carboxylic acid methyl ester trifluoroacetate salt;
2-(3-Methoxy-5-trifluoromethyl-phenylamino)-4-(2-oxo-2,3-dihydro-benzooxazol-5-
ylamino)-pyrimidine-5-carboxylic acid methyl ester trifluoroacetate salt;
2-(4-Carbamoyl-phenylamino)-4-(2-oxo-2,3-dihydro-benzooxazol-5-ylamino)-pyrimidine-
5-carboxylic acid methyl ester trifluoroacetate salt;
2-(4-Fluoro-3-methoxy-5-methyl-phenylamino)-4-(2-oxo-2,3-dihydro-benzooxazol-5-
ylamino)-pyrimidine-5-carboxylic acid methyl ester trifluoroacetate salt;
2-(Naphthalen-2-ylamino)-4-(2-oxo-2,3-dihydro-benzooxazol-5-ylamino)-pyrimidine-5-
carboxylic acid methyl ester trifluoroacetate salt;
2,2,2-trifluoro-N-(1-(5-(5-methyl-4-(2-oxo-2,3-dihydrobenzo[d]oxazol-5-
ylamino)pyrimidin-2-ylamino)pyridin-2-yl)pyrrolidin-3-yl)acetamide;
2,6-Dimethyl-4-[5-methyl-4-(2-oxo-2,3-dihydro-benzooxazol-5-ylamino)-pyrimidin-2-
ylamino]-benzonitrile trifluoroacetate salt;
2-Chloro-4-[5-methyl-4-(2-oxo-2,3-dihydro-benzooxazol-5-ylamino)-pyrimidin-2-
ylamino]-benzamide;
2-fluoro-3-(5-methyl-4-(2-oxo-2,3-dihydrobenzo[d]oxazol-5-ylamino)pyrimidin-2-
ylamino)benzonitrile trifluoroacetate salt;
2-methoxy-4-(5-methyl-4-(2-oxo-2,3-dihydrobenzo[d]oxazol-5-ylamino)pyrimidin-2-
ylamino)-N-phenylbenzamide;
2-methoxy-N,N-dimethyl-4-(5-methyl-4-(2-oxo-2,3-dihydrobenzo[d]oxazol-5-
ylamino)pyrimidin-2-ylamino)benzamide;
2-methoxy-N,N-dimethyl-5-(5-methyl-4-(2-oxo-2,3-dihydrobenzo[d]oxazol-5-
ylamino)pyrimidin-2-ylamino)benzamide;
2-Methyl-4-[5-methyl-4-(2-oxo-2,3-dihydro-benzooxazol-5-ylamino)-pyrimidin-2-
ylamino]-benzoic acid methyl ester;
2-Methyl-4-[5-methyl-4-(2-oxo-2,3-dihydro-benzooxazol-5-ylamino)-pyrimidin-2-
ylamino]-benzoic acid;
2-Methyl-5-[5-methyl-4-(2-oxo-2,3-dihydro-benzooxazol-5-ylamino)-pyrimidin-2-
ylamino]-benzonitrile;
2-Methyl-9-[5-methyl-4-(2-oxo-2,3-dihydro-benzooxazol-5-ylamino)-pyrimidin-2-
ylamino]-3,4,5,6-tetrahydro-2H-benzo[c]azocin-1-one;
3-(4-(2-oxo-2,3-dihydrobenzo[d]oxazol-5-ylamino)pyrimidin-2-ylamino)benzamide
trifluoroacetate salt;
3-(4-(7-fluoro-2-oxo-2,3-dihydrobenzo[d]oxazol-5-ylamino)-5-methylpyrimidin-2-
ylamino)benzenesulfonamide;
3-(5-chloro-4-(2-oxo-2,3-dihydrobenzo[d]oxazol-5-ylamino)pyrimidin-2-ylamino)-2-
fluorobenzonitrile trifluoroacetate salt;
3-(5-fluoro-4-(7-fluoro-2-oxo-2,3-dihydrobenzo[d]oxazol-5-ylamino)pyrimidin-2-
ylamino)benzenesulfonamide trifluoroacetate salt;
3-(5-fluoro-4-(7-methyl-2-oxo-2,3-dihydrobenzo[d]oxazol-5-ylamino)pyrimidin-2-
ylamino)benzenesulfonamide trifluoroacetate salt;
3-(5-methyl-4-(7-methyl-2-oxo-2,3-dihydrobenzo[d]oxazol-5-ylamino)pyrimidin-2-
ylamino)benzenesulfonamide;
3-Chloro-5-[5-methyl-4-(2-oxo-2,3-dihydro-benzooxazol-5-ylamino)-pyrimidin-2-
ylamino]-benzonitrile;
3-methoxy-N,N-dimethyl-5-(5-methyl-4-(2-oxo-2,3-dihydrobenzo[d]oxazol-5-
ylamino)pyrimidin-2-ylamino)benzamide;
4-(2-Oxo-2,3-dihydro-benzooxazol-5-ylamino)-2-(3,4,5-trimethyl-phenylamino)-
pyrimidine-5-carboxylic acid methyl ester trifluoroacetate salt;
4-(2-Oxo-2,3-dihydro-benzooxazol-5-ylamino)-2-(3,4,5-trimethyl-phenylamino)-
pyrimidine-5-carboxylic acid trifluoroacetate salt;
4-(2-oxo-2,3-dihydro-benzooxazol-5-ylamino)-2-phenylamino-pyrimidine-5-carboxylic
acid methyl ester trifluoroacetate salt;
4-(4-(2,3-dihydro-2-oxobenzo[d]oxazol-5-ylamino)-5-methylpyrimidin-2-ylamino)-2-
methoxy-6-methylbenzyl alcohol trifluoroacetate salt;
4-(4-(2,3-dihydro-2-oxobenzo[d]oxazol-5-ylamino)-5-methylpyrimidin-2-ylamino)-2-
methoxy-6-methylbenzyl alcohol;
4-(4-(2,3-dihydro-2-oxobenzo[d]oxazol-5-ylamino)-5-methylpyrimidin-2-ylamino)-2-
methoxy-6-methylbenzoic acid;
4-(4-(2-oxo-2,3-dihydrobenzo[d]oxazol-5-ylamino)pyrimidin-2-ylamino)benzamide
trifluoroacetate salt;
4-(5-chloro-4-(2-oxo-2,3-dihydrobenzo[d]oxazol-5-ylamino)pyrimidin-2-
ylamino)benzamide trifluoroacetate salt;
4-(5-chloro-4-(2-oxo-2,3-dihydrobenzo[d]oxazol-5-ylamino)pyrimidin-2-ylamino)-2,6-
dimethylbenzonitrile]trifluoroacetate salt;
4-(5-fluoro-4-(2-oxo-2,3-dihydrobenzo[d]oxazol-5-ylamino)pyrimidin-2-ylamino)-2,6-
dimethylbenzonitrile;
4-(5-fluoro-4-(2-oxo-2,3-dihydrobenzo[d]oxazol-5-ylamino)pyrimidin-2-ylamino)-N-
propylbenzamide formate salt;
4-(5-fluoro-4-(2-oxo-2,3-dihydrobenzo[d]oxazol-5-ylamino)pyrimidin-2-ylamino)-N-
isopropylbenzamide formate salt;
4-(5-fluoro-4-(2-oxo-2,3-dihydrobenzo[d]oxazol-5-ylamino)pyrimidin-2-ylamino)-N-
phenyl-2-(trifluoromethyl)benzamide formate salt;
4-(5-methyl-4-(2-oxo-2,3-dihydrobenzo[d]oxazol-5-ylamino)pyrimidin-2-ylamino)-N-
phenyl-2-(trifluoromethyl)benzamide;
4-(5-methyl-4-(2-oxo-2,3-dihydrobenzo[d]oxazol-5-ylamino)pyrimidin-2-ylamino)-2-
(trifluoromethyl)benzoic acid;
4-(5-nitro-4-(2-oxo-2,3-dihydrobenzo[d]oxazol-5-ylamino)pyrimidin-2-ylamino)benzamide
trifluoroacetate salt;
4-[5-Methyl-4-(2-oxo-2,3-dihydro-benzooxazol-5-ylamino)-pyrimidin-2-ylamino]-benzoic
acid;
4-[5-Methyl-4-(2-oxo-2,3-dihydro-benzooxazol-5-ylamino)-pyrimidin-2-ylamino]-N-
phenyl-benzamide;
4-[5-Methyl-4-(2-oxo-2,3-dihydro-benzooxazol-5-ylamino)-pyrimidin-2-ylamino]-2-
pyrrolidin-1-yl-benzamide;
4-[5-Methyl-4-(2-oxo-2,3-dihydro-benzooxazol-5-ylamino)-pyrimidin-2-ylamino]-2-(4-
methyl-piperidin-1-yl)-benzamide;
4-[5-Methyl-4-(2-oxo-3-propionyl-2,3-dihydro-benzooxazol-5-ylamino)-pyrimidin-2-
ylamino]-benzamide;
5-(2-(2,3-dimethylphenylamino)-5-methylpyrimidin-4-ylamino)benzo[d]oxazol-2(3H)-one;
5-(2-(2,4-difluoro-3-ethyleneoxyphenylamino)-5-methylpyrimidin-4-
ylamino)benzo[d]oxazol-2(3H)-one;
5-(2-(2,4-difluoro-3-ispropoxyphenylamino)-5-methylpyrimidin-4-
ylamino)benzo[d]oxazol-2(3H)-one;
5-(2-(2,4-difluoro-3-methoxyphenylamino)-5-fluoropyrimidin-4-ylamino)-7-
methylbenzo[d]oxazol-2(3H)-one trifluoroacetate salt;
5-(2-(2,4-difluoro-3-methoxyphenylamino)-5-fluoropyrimidin-4-ylamino)-7-
fluorobenzo[d]oxazol-2(3H)-one trifluoroacetate salt;
5-(2-(2,4-difluoro-3-methoxyphenylamino)-5-methylpyrimidin-4-ylamino)-7-
methylbenzo[d]oxazol-2(3H)-one;
5-(2-(2,4-difluoro-3-methoxyphenylamino)-5-methylpyrimidin-4-ylamino)-7-
fluorobenzo[d]oxazol-2(3H)-one;
5-(2-(2,4-difluoro-3-methoxyphenylamino)-5-nitropyrimidin-4-ylamino)benzo[d]oxazol-
2(3H)-one trifluoroacetate salt;
5-(2-(2,5-dimethylphenylamino)-5-methylpyrimidin-4-ylamino)benzo[d]oxazol-2(3H)-one;
5-(2-(2,6-dimethylphenylamino)-5-methylpyrimidin-4-ylamino)benzo[d]oxazol-2(3H)-one;
5-(2-(2-chloro-5-methylphenylamino)-5-methylpyrimidin-4-ylamino)benzo[d]oxazol-
2(3H)-one;
5-(2-(2-chlorophenylamino)-5-methylpyrimidin-4-ylamino)benzo[d]oxazol-2(3H)-one;
5-(2-(2-ethylphenylamino)-5-methylpyrimidin-4-ylamino)benzo[d]oxazol-2(3H)-one;
5-(2-(2-fluoro-3-(2-methoxyethoxy)-4-methylphenylamino)pyrimidin-4-
ylamino)benzo[d]oxazol-2(3H)-one;
5-(2-(2-fluoro-3,4,5-trimethylphenylamino)-5-methylpyrimidin-4-ylamino)benzo[d]oxazol-
2(3H)-one;
5-(2-(2-fluoro-3,4-bis(2-methoxyethoxy)phenylamino)-5-methylpyrimidin-4-
ylamino)benzo[d]oxazol-2(3H)-one trifluoroacetate salt;
5-(2-(2-fluoro-3,4-d₆-dimethoxyphenylamino)-5-methylpyrimidin-4-
ylamino)benzo[d]oxazol-2(3H)-one;
5-(2-(2-fluoro-3-methoxyphenylamino)pyrimidin-4-ylamino)benzo[d]oxazol-2(3H)-one;
5-(2-(2-fluoro-3-methylphenylamino)-5-methylpyrimidin-4-ylamino)benzo[d]oxazol-
2(3H)-one;
5-(2-(2-fluoro-4,5-dimethylphenylamino)-5-methylpyrimidin-4-ylamino)benzo[d]oxazol-
2(3H)-one;
5-(2-(2-fluoro-4-methoxyphenylamino)-5-methylpyrimidin-4-ylamino)benzo[d]oxazol-
2(3H)-one trifluoroacetate salt;
5-(2-(2-fluoro-4-methyl-3-(trifluoromethyl)phenylamino)-5-methylpyrimidin-4-
ylamino)benzo[d]oxazol-2(3H)-one;
5-(2-(2-fluoro-4-methylphenylamino)-5-methylpyrimidin-4-ylamino)benzo[d]oxazol-
2(3H)-one;
5-(2-(2-fluoro-5-(1-hydroxyethyl)phenylamino)-5-methylpyrimidin-4-
ylamino)benzo[d]oxazol-2(3H)-one;
5-(2-(2-fluoro-5-methoxy-4-methylphenylamino)-5-methylpyrimidin-4-
ylamino)benzo[d]oxazol-2(3H)-one trifluoroacetate salt;
5-(2-(2-fluoro-5-methoxyphenylamino)-5-methylpyrimidin-4-ylamino)benzo[d]oxazol-
2(3H)-one;
5-(2-(2-fluoro-5-methylphenylamino)-5-methylpyrimidin-4-ylamino)benzo[d]oxazol-
2(3H)-one;
5-(2-(2-fluoro-6-methylphenylamino)-5-methylpyrimidin-4-ylamino)benzo[d]oxazol-
2(3H)-one;
5-(2-(3-(1-(3-(diethylamino)pyrrolidin-1-yl)ethyl)phenylamino)-5-methylpyrimidin-4-
ylamino)benzo[d]oxazol-2(3H)-one;
5-(2-(3-(1-(azetidin-1-yl)ethyl)-5-(trifluoromethyl)phenylamino)-5-methylpyrimidin-4-
ylamino)benzo[d]oxazol-2(3H)-one;
5-(2-(3-(1-(azetidin-1-yl)ethyl)-5-methoxyphenylamino)-5-methylpyrimidin-4-
ylamino)benzo[d]oxazol-2(3H)-one;
5-(2-(3-(1-(azetidin-1-yl)ethyl)phenylamino)-5-methylpyrimidin-4-
ylamino)benzo[d]oxazol-2(3H)-one;
5-(2-(3-(1-(benzylamino)ethyl)phenylamino)-5-methylpyrimidin-4-
ylamino)benzo[d]oxazol-2(3H)-one;
5-(2-(3-(1-(cyclobutylamino)ethyl)-5-(trifluoromethyl)phenylamino)-5-methylpyrimidin-4-
ylamino)benzo[d]oxazol-2(3H)-one;
5-(2-(3-(1-(cyclopropylamino)ethyl)-5-(trifluoromethyl)phenylamino)-5-methylpyrimidin-
4-ylamino)benzo[d]oxazol-2(3H)-one;
5-(2-(3-(1-(cyclopropylamino)ethyl)-5-methoxyphenylamino)-5-methylpyrimidin-4-
ylamino)benzo[d]oxazol-2(3H)-one;
5-(2-(3-(1-(diethylamino)ethyl)phenylamino)-5-methylpyrimidin-4-
ylamino)benzo[d]oxazol-2(3H)-one;
5-(2-(3-(1-(ethylamino)ethyl)-5-(trifluoromethyl)phenylamino)-5-methylpyrimidin-4-
ylamino)benzo[d]oxazol-2(3H)-one;
5-(2-(3-(1-(isopropylamino)ethyl)-5-(trifluoromethyl)phenylamino)-5-methylpyrimidin-4-
ylamino)benzo[d]oxazol-2(3H)-one;
5-(2-(3-(1-(isopropylamino)ethyl)-5-methoxyphenylamino)-5-methylpyrimidin-4-
ylamino)benzo[d]oxazol-2(3H)-one;
5-(2-(3-(1-(isopropylamino)ethyl)phenylamino)-5-methylpyrimidin-4-
ylamino)benzo[d]oxazol-2(3H)-one;
5-(2-(3-(1-hydroxyethyl)-5-(trifluoromethyl)phenylamino)-5-methylpyrimidin-4-
ylamino)benzo[d]oxazol-2(3H)-one;
5-(2-(3-(1-hydroxyethyl)-5-methoxyphenylamino)-5-methylpyrimidin-4-
ylamino)benzo[d]oxazol-2(3H)-one;
5-(2-(3-(cyclobutylmethoxy)-5-(trifluoromethyl)phenylamino)-5-methylpyrimidin-4-
ylamino)benzo[d]oxazol-2(3H)-one;
5-(2-(3-(cyclopropylmethoxy)-5-(trifluoromethyl)phenylamino)-5-methylpyrimidin-4-
ylamino)benzo[d]oxazol-2(3H)-one;
5-(2-(3-(diethylamino)-4,5-dimethylphenylamino)-5-methylpyrimidin-4-
ylamino)benzo[d]oxazol-2(3H)-one formate salt;
5-(2-(3-(difluoromethyl)-4-methoxyphenylamino)-5-fluoropyrimidin-4-
ylamino)benzo[d]oxazol-2(3H)-one formate salt;
5-(2-(3-(difluoromethyl)-4-methoxyphenylamino)-5-methylpyrimidin-4-
ylamino)benzo[d]oxazol-2(3H)-one;
5-(2-(3-(difluoromethyl)-4-methylphenylamino)-5-methylpyrimidin-4-
ylamino)benzo[d]oxazol-2(3H)-one;
5-(2-(3-(difluoromethyl)-5-methoxyphenylamino)-5-fluoropyrimidin-4-
ylamino)benzo[d]oxazol-2(3H)-one trifluoroacetate salt;
5-(2-(3-(difluoromethyl)-5-methoxyphenylamino)-5-methylpyrimidin-4-
ylamino)benzo[d]oxazol-2(3H)-one;
5-(2-(3-(difluoromethyl)-5-methoxyphenylamino)-5-methylpyrimidin-4-
ylamino)benzo[d]oxazol-2(3H)-one formate salt;
5-(2-(3-(difluoromethyl)-5-methylphenylamino)-5-methylpyrimidin-4-
ylamino)benzo[d]oxazol-2(3H)-one;
5-(2-(3-(dimethylamino)-4,5-dimethylphenylamino)-5-methylpyrimidin-4-
ylamino)benzo[d]oxazol-2(3H)-one trifluoroacetate salt;
5-(2-(3-(dimethylamino)-4-methylphenylamino)-5-nitropyrimidin-4-
ylamino)benzo[d]oxazol-2(3H)-one trifluoroacetate salt;
5-(2-(3-(ethylamino)-4,5-dimethylphenylamino)-5-methylpyrimidin-4-
ylamino)benzo[d]oxazol-2(3H)-one formate salt;
5-(2-(3-(fluoromethyl)-4-methylphenylamino)-5-methylpyrimidin-4-
ylamino)benzo[d]oxazol-2(3H)-one;
5-(2-(3-(fluoromethyl)-5-methoxyphenylamino)-5-fluoropyrimidin-4-
ylamino)benzo[d]oxazol-2(3H)-one trifluoroacetate salt;
5-(2-(3-(fluoromethyl)-5-methoxyphenylamino)-5-methylpyrimidin-4-
ylamino)benzo[d]oxazol-2(3H)-one;
5-(2-(3-(fluoromethyl)-5-methylphenylamino)-5-methylpyrimidin-4-
ylamino)benzo[d]oxazol-2(3H)-one;
5-(2-(3-(isopropoxymethyl)-4-methoxyphenylamino)-5-methylpyrimidin-4-
ylamino)benzo[d]oxazol-2(3H)-one;
5-(2-(3-(methylsulfonyl)phenylamino)-5-fluoropyrimidin-4-ylamino)-7-
methylbenzo[d]oxazol-2(3H)-one trifluoroacetate salt;
5-(2-(3-(trifluoromethyl)-5-methoxyphenylamino)-5-fluoropyrimidin-4-ylamino)-7-
methylbenzo[d]oxazol-2(3H)-one trifluoroacetate salt;
5-(2-(3,3-dimethyl-2-oxoindolin-6-ylamino)-5-methylpyrimidin-4-ylamino)benzo[d]oxazol-
2(3H)-one;
5-(2-(3,4,5-trimethylphenylamino)-5-((4-methylpiperazin-1-yl)methyl)pyrimidin-4-
ylamino)benzo[d]oxazol-2(3H)-one;
5-(2-(3,4,5-trimethylphenylamino)-5-((piperidin-1-yl)methyl)pyrimidin-4-
ylamino)benzo[d]oxazol-2(3H)-one;
5-(2-(3,4,5-trimethylphenylamino)-5-((pyrrolidin-1-yl)methyl)pyrimidin-4-
ylamino)benzo[d]oxazol-2(3H)-one;
5-(2-(3,4,5-trimethylphenylamino)-5-fluoropyrimidin-4-ylamino)-7-fluorobenzo[d]oxazol-
2(3H)-one trifluoroacetate salt;
5-(2-(3,4,5-trimethylphenylamino)-5-methylpyrimidin-4-ylamino)-3-
methylbenzo[d]oxazol-2(3H)-one trifluoroacetate salt;
5-(2-(3,4,5-trimethylphenylamino)-5-methylpyrimidin-4-ylamino)-7-(benzo[d][1,3]dioxol-
6-yl)benzo[d]oxazol-2(3H)-one;
5-(2-(3,4,5-trimethylphenylamino)-5-methylpyrimidin-4-ylamino)-7-
((dimethylamino)methyl)benzo[d]oxazol-2(3H)-one ditrifluoroacetate salt;
5-(2-(3,4,5-trimethylphenylamino)-5-methylpyrimidin-4-ylamino)-7-((pyrrolidin-1-
yl)methyl)benzo[d]oxazol-2(3H)-one;
5-(2-(3,4,5-trimethylphenylamino)-5-methylpyrimidin-4-ylamino)-7-((piperidin-1-
yl)methyl)benzo[d]oxazol-2(3H)-one;
5-(2-(3,4,5-trimethylphenylamino)-5-methylpyrimidin-4-ylamino)-7-((4-methylpiperazin-1-
yl)methyl)benzo[d]oxazol-2(3H)-one;
5-(2-(3,4,5-trimethylphenylamino)-5-methylpyrimidin-4-ylamino)-7-((piperazin-1-
yl)methyl)benzo[d]oxazol-2(3H)-one ditrifluoroacetate salt;
5-(2-(3,4,5-trimethylphenylamino)-5-methylpyrimidin-4-ylamino)-7-((E)-3-chloroprop-1-
enyl)benzo[d]oxazol-2(3H)-one;
5-(2-(3,4-diethoxy-2-fluorophenylamino)-5-methylpyrimidin-4-ylamino)benzo[d]oxazol-
2(3H)-one;
5-(2-(3,4-dimethoxy-5-methylphenylamino)-5-fluoropyrimidin-4-ylamino)benzo[d]oxazol-
2(3H)-one trifluoroacetate salt;
5-(2-(3,4-dimethoxy-5-methylphenylamino)-5-fluoropyrimidin-4-ylamino)-7-
methylbenzo[d]oxazol-2(3H)-one trifluoroacetate salt;
5-(2-(3,4-dimethoxy-5-methylphenylamino)-5-fluoropyrimidin-4-ylamino)-7-
fluorobenzo[d]oxazol-2(3H)-one trifluoroacetate salt;
5-(2-(3,4-dimethoxy-5-methylphenylamino)-5-methylpyrimidin-4-ylamino)benzo[d]oxazol-
2(3H)-one;
5-(2-(3,4-dimethoxy-5-methylphenylamino)-5-methylpyrimidin-4-ylamino)-7-
methylbenzo[d]oxazol-2(3H)-one;
5-(2-(3,4-dimethoxy-5-methylphenylamino)-5-methylpyrimidin-4-ylamino)-7-
fluorobenzo[d]oxazol-2(3H)-one;
5-(2-(3,5-difluorophenylamino)pyrimidin-4-ylamino)benzo[d]oxazol-2(3H)-one
trifluoroacetate salt;
5-(2-(3,5-dimethoxy-4-methylphenylamino)-5-fluoropyrimidin-4-ylamino)benzo[d]oxazol-
2(3H)-one trifluoroacetate salt;
5-(2-(3,5-dimethoxy-4-methylphenylamino)-5-methylpyrimidin-4-ylamino)benzo[d]oxazol-
2(3H)-one;
5-(2-(3,5-dimethyl-4-(2-morpholinoethoxy)phenylamino)-5-methylpyrimidin-4-
ylamino)benzo[d]oxazol-2(3H)-one;
5-(2-(3,5-dimethyl-4-d₃-methyl)phenylamino)pyrimidin-4-ylamino)benzo[d]oxazol-2(3H)-
one trifluoroacetate salt;
5-(2-(3,5-dimethylphenylamino)-5-fluoropyrimidin-4-ylamino)-7-fluorobenzo[d]oxazol-
2(3H)-one trifluoroacetate salt;
5-(2-(3,5-dimethylphenylamino)-5-methylpyrimidin-4-ylamino)-7-fluorobenzo[d]oxazol-
2(3H)-one;
5-(2-(3,5-dimethylphenylamino)-5-methylpyrimidin-4-ylamino)-7-methylbenzo[d]oxazol-
2(3H)-one;
5-(2-(3-acetyl-5-(trifluoromethyl)phenylamino)-5-methylpyrimidin-4-
ylamino)benzo[d]oxazol-2(3H)-one;
5-(2-(3-acetyl-5-methoxyphenylamino)-5-methylpyrimidin-4-ylamino)benzo[d]oxazol-
2(3H)-one;
5-(2-(3-chloro-4,5-dimethoxyphenylamino)-5-fluoropyrimidin-4-ylamino)benzo[d]oxazol-
2(3H)-one trifluoroacetate salt;
5-(2-(3-chloro-4,5-dimethoxyphenylamino)-5-methylpyrimidin-4-ylamino)benzo[d]oxazol-
2(3H)-one;
5-(2-(3-chloro-4-fluoro-5-(trifluoromethyl)phenylamino)-5-methylpyrimidin-4-
ylamino)benzo[d]oxazol-2(3H)-one;
5-(2-(3-chloro-4-methoxy-5-methylphenylamino)-5-methylpyrimidin-4-
ylamino)benzo[d]oxazol-2(3H)-one;
5-(2-(3-cyclobutoxy-5-(trifluoromethyl)phenylamino)-5-methylpyrimidin-4-
ylamino)benzo[d]oxazol-2(3H)-one;
5-(2-(3-deuteratedmethoxy-5-(trifluoromethyl)phenylamino)-5-methylpyrimidin-4-
ylamino)benzo[d]oxazol-2(3H)-one;
5-(2-(3-ethoxy-4,5-dimethylphenylamino)-5-methylpyrimidin-4-ylamino)benzo[d]oxazol-
2(3H)-one ditrifluoroacetate salt;
5-(2-(3-ethylphenylamino)-5-methylpyrimidin-4-ylamino)benzo[d]oxazol-2(3H)-one;
5-(2-(3-fluoro-4-(pyridin-3-yl)phenylamino)-5-methylpyrimidin-4-
ylamino)benzo[d]oxazol-2(3H)-one trifluoroacetic acid salt;
5-(2-(3-fluoro-4-(pyridin-4-yl)phenylamino)-5-methylpyrimidin-4-
ylamino)benzo[d]oxazol-2(3H)-one;
5-(2-(3-fluoro-5-(4-methylpiperazin-1-yl)phenylamino)-5-fluoropyrimidin-4-
ylamino)benzo[d]oxazol-2(3H)-one ditrifluoroacetate salt;
5-(2-(3-fluoro-5-(4-methylpiperazin-1-yl)phenylamino)-5-methylpyrimidin-4-
ylamino)benzo[d]oxazol-2(3H)-one;
5-(2-(3-fluoro-5-methoxy-4-methylphenylamino)-5-methylpyrimidin-4-
ylamino)benzo[d]oxazol-2(3H)-one trifluoroacetate salt;
5-(2-(3-fluoro-5-morpholinophenylamino)-5-fluoropyrimidin-4-ylamino)benzo[d]oxazol-
2(3H)-one ditrifluoroacetate salt;
5-(2-(3-fluoro-5-morpholinophenylamino)-5-methylpyrimidin-4-ylamino)benzo[d]oxazol-
2(3H)-one;
5-(2-(3-hydroxy-4,5-dimethylphenylamino)-5-methylpyrimidin-4-ylamino)benzo[d]oxazol-
2(3H)-one trifluoroacetate salt;
5-(2-(3-methoxy-4-(4-methylpiperazine-1-carbonyl)phenylamino)-5-methylpyrimidin-4-
ylamino)benzo[d]oxazol-2(3H)-one;
5-(2-(3-methoxy-4-(morpholine-4-carbonyl)phenylamino)-5-methylpyrimidin-4-
ylamino)benzo[d]oxazol-2(3H)-one;
5-(2-(3-methoxy-4-(pyridin-3-yl)phenylamino)-5-methylpyrimidin-4-
ylamino)benzo[d]oxazol-2(3H)-one formate salt;
5-(2-(3-methoxy-4-(pyridin-4-yl)phenylamino)-5-fluoropyrimidin-4-
ylamino)benzo[d]oxazol-2(3H)-one formate salt;
5-(2-(3-methoxy-4-(pyridin-4-yl)phenylamino)-5-methylpyrimidin-4-
ylamino)benzo[d]oxazol-2(3H)-one formate salt;
5-(2-(3-methoxy-4-(pyrrolidine-1-carbonyl)phenylamino)-5-methylpyrimidin-4-
ylamino)benzo[d]oxazol-2(3H)-one;
5-(2-(3-methoxy-4,5-dimethylphenylamino)-5-fluoropyrimidin-4-ylamino)benzo[d]oxazol-
2(3H)-one trifluoroacetate salt;
5-(2-(3-methoxy-4,5-dimethylphenylamino)-5-methylpyrimidin-4-ylamino)benzo[d]oxazol-
2(3H)-one benzenesulfonic acid salt;
5-(2-(3-methoxy-4,5-dimethylphenylamino)-5-methylpyrimidin-4-ylamino)-7-
methylbenzo[d]oxazol-2(3H)-one trifluoroacetate trifluoroacetate salt;
5-(2-(3-methoxy-4,5-dimethylphenylamino)-5-methylpyrimidin-4-ylamino)-7-
fluorobenzo[d]oxazol-2(3H)-one trifluoroacetate trifluoroacetate salt;
5-(2-(3-methoxy-4,5-dimethylphenylamino)-5-methylpyrimidin-4-ylamino)benzo[d]oxazol-
2(3H)-one;
5-(2-(3-methoxy-4,5-dimethylphenylamino)-5-methylpyrimidin-4-ylamino)-3-
methylbenzo[d]oxazol-2(3H)-one trifluoroacetate salt;
5-(2-(3-methoxy-4,5-dimethylphenylamino)-5-methylpyrimidin-4-ylamino)benzo[d]oxazol-
2(3H)-one;
5-(2-(3-methoxy-4,5-dimethylphenylamino)-6-D-5-trideuteromethylpyrimidin-4-
ylamino)benzo[d]oxazol-2(3H)-one;
5-(2-(3-methoxy-4-methylphenylamino)-5-nitropyrimidin-4-ylamino)benzo[d]oxazol-
2(3H)-one trifluoroacetate salt;
5-(2-(3-methoxy-5-(1-(methylamino)ethyl)phenylamino)-5-methylpyrimidin-4-
ylamino)benzo[d]oxazol-2(3H)-one;
5-(2-(3-methoxy-5-(1-(propylamino)ethyl)phenylamino)-5-methylpyrimidin-4-
ylamino)benzo[d]oxazol-2(3H)-one;
5-(2-(3-methoxy-5-(1-(pyrrolidin-1-yl)ethyl)phenylamino)-5-methylpyrimidin-4-
ylamino)benzo[d]oxazol-2(3H)-one;
5-(2-(3-methoxy-5-(4-methylpiperazine-1-carbonyl)phenylamino)-5-methylpyrimidin-4-
ylamino)benzo[d]oxazol-2(3H)-one;
5-(2-(3-methoxy-5-(morpholine-4-carbonyl)phenylamino)-5-methylpyrimidin-4-
ylamino)benzo[d]oxazol-2(3H)-one;
5-(2-(3-methoxy-5-(pyrrolidine-1-carbonyl)phenylamino)-5-methylpyrimidin-4-
ylamino)benzo[d]oxazol-2(3H)-one;
5-(2-(3-methoxy-5-(trifluoromethyl)phenylamino)-5-methylpyrimidin-4-ylamino)-7-
methylbenzo[d]oxazol-2(3H)-one;
5-(2-(3-methoxy-5-(trifluoromethyl)phenylamino)-5-nitropyrimidin-4-
ylamino)benzo[d]oxazol-2(3H)-one trifluoroacetate salt;
5-(2-(3-methoxy-5-methylphenylamino)-5-fluoropyrimidin-4-ylamino)-7-
methylbenzo[d]oxazol-2(3H)-one trifluoroacetate salt;
5-(2-(3-methoxy-5-methylphenylamino)-5-fluoropyrimidin-4-ylamino)-7-
fluorobenzo[d]oxazol-2(3H)-one trifluoroacetate salt;
5-(2-(3-methoxy-5-methylphenylamino)-5-methylpyrimidin-4-ylamino)benzo[d]oxazol-
2(3H)-one;
5-(2-(3-methoxy-5-methylphenylamino)-5-methylpyrimidin-4-ylamino)-7-
methylbenzo[d]oxazol-2(3H)-one;
5-(2-(3-methyl-4-trideuteromethoxyphenylamino)-5-methylpyrimidin-4-
ylamino)benzo[d]oxazol-2(3H)-one;
5-(2-(3-methyl-5-trideuteromethoxyphenylamino)-5-methylpyrimidin-4-
ylamino)benzo[d]oxazol-2(3H)-one;
5-(2-(3-trideuteromethoxy-4,5-dimethylphenylamino)-6-D-5-trideuteromethylpyrimidin-4-
ylamino)benzo[d]oxazol-2(3H)-one;
5-(2-(4-(1-(3-(diethylamino)pyrrolidin-1-yl)ethyl)phenylamino)-5-methylpyrimidin-4-
ylamino)benzo[d]oxazol-2(3H)-one;
5-(2-(4-(1-(azetidin-1-yl)ethyl)phenylamino)-5-methylpyrimidin-4-
ylamino)benzo[d]oxazol-2(3H)-one;
5-(2-(4-(1-(benzylamino)ethyl)phenylamino)-5-methylpyrimidin-4-
ylamino)benzo[d]oxazol-2(3H)-one;
5-(2-(4-(1-(cyclopropylamino)ethyl)phenylamino)-5-fluoropyrimidin-4-
ylamino)benzo[d]oxazol-2(3H)-one formate salt;
5-(2-(4-(1-(cyclopropylamino)ethyl)phenylamino)-5-methylpyrimidin-4-
ylamino)benzo[d]oxazol-2(3H)-one;
5-(2-(4-(1-(isopropylamino)ethyl)phenylamino)-5-methylpyrimidin-4-
ylamino)benzo[d]oxazol-2(3H)-one;
5-(2-(4-(2-morpholinoethoxy)-3,5-dimethylphenylamino)-5-fluoropyrimidin-4-
ylamino)benzo[d]oxazol-2(3H)-one ditrifluoroacetate salt;
5-(2-(4-(6-(3-(dimethylamino)propoxy)pyridin-3-yl)phenylamino)-5-methylpyrimidin-4-
ylamino)benzo[d]oxazol-2(3H)-one trifluoroacetate salt;
5-(2-(4-(6-chloropyridin-3-yl)phenylamino)-5-methylpyrimidin-4-ylamino)benzo[d]oxazol-
2(3H)-one trifluoroacetate salt;
5-(2-(4-(aminomethyl)phenylamino)-5-methylpyrimidin-4-ylamino)benzo[d]oxazol-2(3H)-
one;
5-(2-(4-(difluoromethoxy)-3-(difluoromethyl)phenylamino)-5-methylpyrimidin-4-
ylamino)benzo[d]oxazol-2(3H)-one formate salt;
5-(2-(4-(difluoromethoxy)-3-(fluoromethyl)phenylamino)-5-methylpyrimidin-4-
ylamino)benzo[d]oxazol-2(3H)-one formate salt;
5-(2-(4-(hydroxymethyl)-3-methoxy-5-methylphenylamino)-6-D-5-
trideuteromethylpyrimidin-4-ylamino)benzo[d]oxazol-2(3H)-one;
5-(2-(4-acetylphenylamino)-5-fluoropyrimidin-4-ylamino)benzo[d]oxazol-2(3H)-one
formate salt;
5-(2-(4-chloro-3-methoxy-5-methylphenylamino)-5-methylpyrimidin-4-
ylamino)benzo[d]oxazol-2(3H)-one trifluoroacetate salt;
5-(2-(4-d₃-methoxy-3-(trifluoromethyl)phenylamino)-5-methylpyrimidin-4-
ylamino)benzo[d]oxazol-2(3H)-one;
5-(2-(4-ethylphenylamino)-5-methylpyrimidin-4-ylamino)benzo[d]oxazol-2(3H)-one;
5-(2-(4-fluoro-3-(methylsulfonyl)phenylamino)-5-fluoropyrimidin-4-
ylamino)benzo[d]oxazol-2(3H)-one trifluoroacetate salt;
5-(2-(4-fluoro-3-(methylsulfonyl)phenylamino)-5-fluoropyrimidin-4-ylamino)-7-
methylbenzo[d]oxazol-2(3H)-one trifluoroacetate salt;
5-(2-(4-fluoro-3-(methylsulfonyl)phenylamino)-5-methylpyrimidin-4-
ylamino)benzo[d]oxazol-2(3H)-one;
5-(2-(4-fluoro-3-(methylsulfonyl)phenylamino)-5-methylpyrimidin-4-ylamino)-7-
methylbenzo[d]oxazol-2(3H)-one;
5-(2-(4-fluoro-3-(pyridin-3-yl)phenylamino)-5-fluoropyrimidin-4-ylamino)benzo[d]oxazol-
2(3H)-one;
5-(2-(4-fluoro-3-(pyridin-3-yl)phenylamino)-5-methylpyrimidin-4-
ylamino)benzo[d]oxazol-2(3H)-one;
5-(2-(4-fluoro-3-(pyridin-4-yl)phenylamino)-5-fluoropyrimidin-4-ylamino)benzo[d]oxazol-
2(3H)-one;
5-(2-(4-fluoro-3-(pyridin-4-yl)phenylamino)-5-methylpyrimidin-4-
ylamino)benzo[d]oxazol-2(3H)-one;
5-(2-(4-fluoro-3,5-dimethylphenylamino)-5-(hydroxymethyl)pyrimidin-4-
ylamino)benzo[d]oxazol-2(3H)-one trifluoroacetate salt;
5-(2-(4-fluoro-3,5-dimethylphenylamino)-5-fluoropyrimidin-4-ylamino)-7-
fluorobenzo[d]oxazol-2(3H)-one trifluoroacetate salt;
5-(2-(4-fluoro-3,5-dimethylphenylamino)-5-methylpyrimidin-4-ylamino)-7-
methylbenzo[d]oxazol-2(3H)-one;
5-(2-(4-fluoro-3-hydroxy-5-methylphenylamino)-5-methylpyrimidin-4-
ylamino)benzo[d]oxazol-2(3H)-one trifluoroacetate salt;
5-(2-(4-fluoro-3-methoxy-5-methylphenylamino)-5-(hydroxymethyl)pyrimidin-4-
ylamino)benzo[d]oxazol-2(3H)-one trifluoroacetate salt;
5-(2-(4-fluoro-3-methoxy-5-methylphenylamino)-5-fluoropyrimidin-4-
ylamino)benzo[d]oxazol-2(3H)-one trifluoroacetate salt;
5-(2-(4-fluoro-3-methoxy-5-methylphenylamino)-5-fluoropyrimidin-4-ylamino)-7-
methylbenzo[d]oxazol-2(3H)-one trifluoroacetate salt;
5-(2-(4-fluoro-3-methoxy-5-methylphenylamino)-5-fluoropyrimidin-4-ylamino)-7-
fluorobenzo[d]oxazol-2(3H)-one trifluoroacetate salt;
5-(2-(4-fluoro-3-methoxy-5-methylphenylamino)-5-methylpyrimidin-4-
ylamino)benzo[d]oxazol-2(3H)-one;
5-(2-(4-fluoro-3-methoxy-5-methylphenylamino)-5-methylpyrimidin-4-
ylamino)benzo[d]oxazol-2(3H)-one methane sulfonic acid salt;
5-(2-(4-fluoro-3-methoxy-5-methylphenylamino)-5-methylpyrimidin-4-
ylamino)benzo[d]oxazol-2(3H)-one benzenesulfonic acid salt;
5-(2-(4-fluoro-3-methoxy-5-methylphenylamino)-5-methylpyrimidin-4-
ylamino)benzo[d]oxazol-2(3H)-one sulfuric acid salt;
5-(2-(4-fluoro-3-methoxy-5-methylphenylamino)-5-methylpyrimidin-4-ylamino)-3-
methylbenzo[d]oxazol-2(3H)-one trifluoroacetate salt;
5-(2-(4-fluoro-3-methoxy-5-methylphenylamino)-5-methylpyrimidin-4-ylamino)-7-
methylbenzo[d]oxazol-2(3H)-one;
5-(2-(4-fluoro-3-methoxy-5-methylphenylamino)-6-D-5-trideuteromethylpyrimidin-4-
ylamino)benzo[d]oxazol-2(3H)-one;
5-(2-(4-fluoro-3-trideuteromethoxy-5-methylphenylamino)-6-D-5-
trideuteromethylpyrimidin-4-ylamino)benzo[d]oxazol-2(3H)-one;
5-(2-(4-fluoro-3-trideuteromethoxy-5-methylphenylamino)-6-D-5-
trideuteromethylpyrimidin-4-ylamino)-2-oxobenzo[d]oxazol-3(2H)-yl)methyl di-tert-butyl
phosphate;
5-(2-(4-fluoro-3-trideuteromethoxy-5-methylphenylamino)-6-D-5-
trideuteromethylpyrimidin-4-ylamino)-2-oxobenzo[d]oxazol-3(2H)-yl)methyl tert-butyl
hydrogen phosphate;
5-(2-(4-fluoro-3-trideuteromethoxy-5-methylphenylamino)-6-D-5-
trideuteromethylpyrimidin-4-ylamino)-2-oxobenzo[d]oxazol-3(2H)-yl)methyl dihydrogen
phosphate;
5-(2-(4-isopropylphenylamino)-5-fluoropyrimidin-4-ylamino)benzo[d]oxazol-2(3H)-one
trifluoroacetate salt;
5-(2-(4-isopropylphenylamino)-5-methylpyrimidin-4-ylamino)benzo[d]oxazol-2(3H)-one;
5-(2-(4-methoxy-3-(4-methylpiperazine-1-carbonyl)phenylamino)-5-methylpyrimidin-4-
ylamino)benzo[d]oxazol-2(3H)-one;
5-(2-(4-methoxy-3-(morpholine-4-carbonyl)phenylamino)-5-methylpyrimidin-4-
ylamino)benzo[d]oxazol-2(3H)-one;
5-(2-(4-methoxy-3-(pyridin-3-yl)phenylamino)-5-fluoropyrimidin-4-
ylamino)benzo[d]oxazol-2(3H)-one formate salt;
5-(2-(4-methoxy-3-(pyridin-3-yl)phenylamino)-5-methylpyrimidin-4-
ylamino)benzo[d]oxazol-2(3H)-one formate salt;
5-(2-(4-methoxy-3-(pyridin-4-yl)phenylamino)-5-fluoropyrimidin-4-
ylamino)benzo[d]oxazol-2(3H)-one formate salt;
5-(2-(4-methoxy-3-(pyridin-4-yl)phenylamino)-5-methylpyrimidin-4-
ylamino)benzo[d]oxazol-2(3H)-one formate salt;
5-(2-(4-methoxy-3-(pyrrolidine-1-carbonyl)phenylamino)-5-methylpyrimidin-4-
ylamino)benzo[d]oxazol-2(3H)-one;
5-(2-(4-methoxy-3-methylphenylamino)-5-fluoropyrimidin-4-ylamino)-7-
methylbenzo[d]oxazol-2(3H)-one trifluoroacetate salt;
5-(2-(4-methoxy-3-methylphenylamino)-5-fluoropyrimidin-4-ylamino)-7-
fluorobenzo[d]oxazol-2(3H)-one trifluoroacetate salt;
5-(2-(4-methoxy-3-methylphenylamino)-5-methylpyrimidin-4-ylamino)benzo[d]oxazol-
2(3H)-one;
5-(2-(4-methoxy-3-methylphenylamino)-5-methylpyrimidin-4-ylamino)-7-
methylbenzo[d]oxazol-2(3H)-one;
5-(2-(4-methoxyphenylamino)-5-fluoropyrimidin-4-ylamino)benzo[d]oxazol-2(3H)-one
formate salt;
5-(2-(4-methoxyphenylamino)-5-methylpyrimidin-4-ylamino)benzo[d]oxazol-2(3H)-one;
5-(2-(4-methyl-3-(methylsulfonyl)phenylamino)-5-fluoropyrimidin-4-
ylamino)benzo[d]oxazol-2(3H)-one trifluoroacetate salt;
5-(2-(4-methyl-3-(pyridin-3-yl)phenylamino)-5-fluoropyrimidin-4-
ylamino)benzo[d]oxazol-2(3H)-one;
5-(2-(4-methyl-3-(pyridin-4-yl)phenylamino)-5-fluoropyrimidin-4-
ylamino)benzo[d]oxazol-2(3H)-one;
5-(2-(4-tert-butylphenylamino)-5-fluoropyrimidin-4-ylamino)benzo[d]oxazol-2(3H)-one
trifluoroacetate salt;
5-(2-(4-tert-butylphenylamino)-5-methylpyrimidin-4-ylamino)benzo[d]oxazol-2(3H)-one;
5-(2-(5-acetyl-2-fluorophenylamino)-5-methylpyrimidin-4-ylamino)benzo[d]oxazol-2(3H)-
one;
5-(2-(5-bromopyridin-3-ylamino)-5-methylpyrimidin-4-ylamino)benzo[d]oxazol-2(3H)-
one;
5-(2-(6-(3-(cyclopropylmethylamino)pyrrolidin-1-yl)pyridin-3-ylamino)-5-
methylpyrimidin-4-ylamino)benzo[d]oxazol-2(3H)-one;
5-(2-(6-(3-(diethylamino)pyrrolidin-1-yl)pyridin-3-ylamino)-5-methylpyrimidin-4-
ylamino)benzo[d]oxazol-2(3H)-one;
5-(2-(6-(3-(dimethylamino)pyrrolidin-1-yl)pyridin-3-ylamino)-5-methylpyrimidin-4-
ylamino)benzo[d]oxazol-2(3H)-one;
5-(2-(6-(3,8-diaza-bicyclo[3.2.1]octan-3-yl)-5-methylpyridin-3-ylamino)-5-
methylpyrimidin-4-ylamino)benzo[d]oxazol-2(3H)-one;
5-(2-(6-(4,4-difluoropiperidin-1-yl)pyridin-3-ylamino)-5-methylpyrimidin-4-
ylamino)benzo[d]oxazol-2(3H)-one;
5-(2-(6-(4,4-dimethylpiperidin-1-yl)pyridin-3-ylamino)-5-methylpyrimidin-4-
ylamino)benzo[d]oxazol-2(3H)-one;
5-(2-(6-(4-methylpiperazin-1-yl)pyridin-3-ylamino)-5-(trifluoromethyl)pyrimidin-4-
ylamino)benzo[d]oxazol-2(3H)-one;
5-(2-(6-(8-(cyclopropylmethyl)-8-aza-bicyclo[3.2.1]octan-3-yl)-5-methylpyridin-3-
ylamino)-5-methylpyrimidin-4-ylamino)benzo[d]oxazol-2(3H)-one;
5-(2-(6-(8-aza-bicyclo[3.2.1]octan-3-yl)-5-methylpyridin-3-ylamino)-5-methylpyrimidin-4-
ylamino)benzo[d]oxazol-2(3H)-one;
5-(2-(6-(cyclopropylmethylamino)pyridin-3-ylamino)-5-methylpyrimidin-4-
ylamino)benzo[d]oxazol-2(3H)-one;
5-(2-(naphthalen-2-ylamino)-5-nitropyrimidin-4-ylamino)benzo[d]oxazol-2(3H)-one
trifluoroacetate salt;
5-(2-(o-toluidino)-5-methylpyrimidin-4-ylamino)benzo[d]oxazol-2(3H)-one;
5-(2-(p-toluidino)-5-methylpyrimidin-4-ylamino)benzo[d]oxazol-2(3H)-one;
5-(2-(p-tolylamino)-5-fluoropyrimidin-4-ylamino)benzo[d]oxazol-2(3H)-one
trifluoroacetate salt;
5-(5-((diethylamino)methyl)-2-(3,4,5-trimethylphenylamino)pyrimidin-4-
ylamino)benzo[d]oxazol-2(3H)-one ditrifluoroacetate salt;
5-(5-(hydroxymethyl)-2-(3-methoxy-4,5-dimethylphenylamino)pyrimidin-4-
ylamino)benzo[d]oxazol-2(3H)-one trifluoroacetate salt;
5-(5-bromo-2-(2-fluoro-3-methoxyphenylamino)pyrimidin-4-ylamino)benzo[d]oxazol-
2(3H)-one trifluoroacetate salt;
5-(5-bromo-2-(3,4,5-trimethylphenylamino)pyrimidin-4-ylamino)benzo[d]oxazol-2(3H)-
one formate salt;
5-(5-chloro-2-(2,4-difluoro-3-methoxyphenylamino)pyrimidin-4-ylamino)benzo[d]oxazol-
2(3H)-one trifluoroacetate salt;
5-(5-chloro-2-(2-fluoro-3-(2-methoxyethoxy)-4-methylphenylamino)pyrimidin-4-
ylamino)benzo[d]oxazol-2(3H)-one trifluoroacetate salt;
5-(5-chloro-2-(2-fluoro-3,4-bis(2-methoxyethoxy)phenylamino)pyrimidin-4-
ylamino)benzo[d]oxazol-2(3H)-one trifluoroacetate salt;
5-(5-chloro-2-(2-fluoro-3,4-dimethoxyphenylamino)pyrimidin-4-ylamino)benzo[d]oxazol-
2(3H)-one trifluoroacetate salt;
5-(5-chloro-2-(2-fluoro-3-methoxy-4-methylphenylamino)pyrimidin-4-
ylamino)benzo[d]oxazol-2(3H)-one;
5-(5-chloro-2-(2-fluoro-3-methoxyphenylamino)pyrimidin-4-ylamino)benzo[d]oxazol-
2(3H)-one trifluoroacetate salt;
5-(5-chloro-2-(2-fluoro-4-methoxyphenylamino)pyrimidin-4-ylamino)benzo[d]oxazol-
2(3H)-one trifluoroacetate salt;
5-(5-chloro-2-(3-(diethylamino)-4,5-dimethylphenylamino)pyrimidin-4-
ylamino)benzo[d]oxazol-2(3H)-one formate salt;
5-(5-chloro-2-(3-(dimethylamino)-4,5-dimethylphenylamino)pyrimidin-4-
ylamino)benzo[d]oxazol-2(3H)-one trifluoroacetate salt;
5-(5-chloro-2-(3-(dimethylamino)-4-methylphenylamino)pyrimidin-4-
ylamino)benzo[d]oxazol-2(3H)-one;
5-(5-chloro-2-(3-(ethylamino)-4,5-dimethylphenylamino)pyrimidin-4-
ylamino)benzo[d]oxazol-2(3H)-one formate salt;
5-(5-chloro-2-(3,4,5-trimethylphenylamino)pyrimidin-4-ylamino)benzo[d]oxazol-2(3H)-
one trifluoroacetate salt;
5-(5-chloro-2-(3,4-dimethylphenylamino)pyrimidin-4-ylamino)benzo[d]oxazol-2(3H)-one
trifluoroacetate salt;
5-(5-chloro-2-(3,5-difluorophenylamino)pyrimidin-4-ylamino)benzo[d]oxazol-2(3H)-one
trifluoroacetate salt;
5-(5-chloro-2-(3,5-dimethyl-4-d₃-methyl)phenylamino)pyrimidin-4-
ylamino)benzo[d]oxazol-2(3H)-one trifluoroacetate salt;
5-(5-chloro-2-(3,5-dimethylphenylamino)pyrimidin-4-ylamino)benzo[d]oxazol-2(3H)-one
trifluoroacetate salt;
5-(5-chloro-2-(3-chloro-5-fluorophenylamino)pyrimidin-4-ylamino)benzo[d]oxazol-2(3H)-
one formate salt;
5-(5-chloro-2-(3-ethoxy-4,5-dimethylphenylamino)pyrimidin-4-ylamino)benzo[d]oxazol-
2(3H)-one ditrifluoroacetate salt;
5-(5-chloro-2-(3-methoxy-4,5-dimethylphenylamino)pyrimidin-4-ylamino)benzo[d]oxazol-
2(3H)-one;
5-(5-chloro-2-(3-methoxy-4-methylphenylamino)pyrimidin-4-ylamino)benzo[d]oxazol-
2(3H)-one trifluoroacetate salt;
5-(5-chloro-2-(3-methoxy-5-(trifluoromethyl)phenylamino)pyrimidin-4-
ylamino)benzo[d]oxazol-2(3H)-one;
5-(5-chloro-2-(4-(trifluoromethyl)phenylamino)pyrimidin-4-ylamino)benzo[d]oxazol-
2(3H)-one trifluoroacetate salt;
5-(5-chloro-2-(4-ethyl-2-fluoro-3-methoxyphenylamino)pyrimidin-4-
ylamino)benzo[d]oxazol-2(3H)-one trifluoroacetate salt;
5-(5-chloro-2-(4-methyl-3-(trifluoromethyl)phenylamino)pyrimidin-4-
ylamino)benzo[d]oxazol-2(3H)-one;
5-(5-chloro-2-(phenylamino)pyrimidin-4-ylamino)benzo[d]oxazol-2(3H)-one
trifluoroacetate salt;
5-(5-fluoro-2-(1-oxo-1,2,3,4-tetrahydroisoquinolin-6-ylamino)pyrimidin-4-
ylamino)benzo[d]oxazol-2(3H)-one trifluoroacetate salt;
5-(5-fluoro-2-(1-oxo-2,3-dihydro-1H-benzo[c]azepin-7-ylamino)pyrimidin-4-
ylamino)benzo[d]oxazol-2(3H)-one formate salt;
5-(5-fluoro-2-(2-fluoro-3-(2-methoxyethoxy)-4-methylphenylamino)pyrimidin-4-
ylamino)benzo[d]oxazol-2(3H)-one trifluoroacetate salt;
5-(5-fluoro-2-(2-fluoro-3-methoxyphenylamino)pyrimidin-4-ylamino)benzo[d]oxazol-
2(3H)-one trifluoroacetate salt;
5-(5-fluoro-2-(2-fluoro-5-methoxy-4-methylphenylamino)pyrimidin-4-
ylamino)benzo[d]oxazol-2(3H)-one trifluoroacetate salt;
5-(5-fluoro-2-(2-fluoro-5-methoxyphenylamino)pyrimidin-4-ylamino)benzo[d]oxazol-
2(3H)-one;
5-(5-fluoro-2-(2-methyl-1-oxo-2,3-dihydro-1H-benzo[c]azepin-7-ylamino)pyrimidin-4-
ylamino)benzo[d]oxazol-2(3H)-one formate salt;
5-(5-fluoro-2-(2-morpholinopyridin-4-ylamino)pyrimidin-4-ylamino)benzo[d]oxazol-
2(3H)-one ditrifluoroacetate salt;
5-(5-fluoro-2-(3,5-dimethyl-4-d₃-methyl)phenylamino)pyrimidin-4-
ylamino)benzo[d]oxazol-2(3H)-one trifluoroacetate salt;
5-(5-fluoro-2-(3-d₃-methoxy-5-methylphenylamino)pyrimidin-4-ylamino)benzo[d]oxazol-
2(3H)-one trifluoroacetate salt;
5-(5-fluoro-2-(3-fluoro-5-methoxy-4-methylphenylamino)pyrimidin-4-
ylamino)benzo[d]oxazol-2(3H)-one trifluoroacetate salt;
5-(5-fluoro-2-(3-methoxy-4,5-dimethylphenylamino)pyrimidin-4-ylamino)-7-
methylbenzo[d]oxazol-2(3H)-one trifluoroacetate salt;
5-(5-fluoro-2-(4-aminosulfonylphenylamino)pyrimidin-4-ylamino)-1H-benzo[d]imidazol-
2(3H)-one;
5-(5-fluoro-2-(5-oxo-6,7,8,9-tetrahydro-5H-benzo[7]annulen-2-ylamino)pyrimidin-4-
ylamino)benzo[d]oxazol-2(3H)-one formate salt;
5-(5-fluoro-2-(7-(pyrrolidin-1-yl)-6,7,8,9-tetrahydro-5H-benzo[7]annulen-2-
ylamino)pyrimidin-4-ylamino)benzo[d]oxazol-2(3H)-one diformate salt;
5-(5-fluoro-2-(7-oxo-6,7,8,9-tetrahydro-5H-benzo[7]annulen-2-ylamino)pyrimidin-4-
ylamino)benzo[d]oxazol-2(3H)-one diformate salt;
5-(5-fluoro-4-(2-oxo-2,3-dihydrobenzo[d]oxazol-5-ylamino)pyrimidin-2-ylamino)-2-
methylbenzonitrile formate salt;
5-(5-methyl-2-(1-methyl-2-oxo-2,3,4,5-tetrahydro-1H-benzo[b]azepin-8-
ylamino)pyrimidin-4-ylamino)benzo[d]oxazol-2(3H)-one;
5-(5-methyl-2-(1-methyl-2-oxo-2,3,4,5-tetrahydro-1H-benzo[b]azepin-7-
ylamino)pyrimidin-4-ylamino)benzo[d]oxazol-2(3H)-one;
5-(5-methyl-2-(1-methyl-2-oxo-2,3-dihydro-1H-benzo[d]imidazol-5-ylamino)pyrimidin-4-
ylamino)benzo[d]oxazol-2(3H)-one;
5-(5-methyl-2-(1-methyl-2-oxoindolin-5-ylamino)pyrimidin-4-ylamino)benzo[d]oxazol-
2(3H)-one;
5-(5-methyl-2-(2-methyl-1-oxo-2,3,4,5-tetrahydro-1H-benzo[c]azepin-7-
ylamino)pyrimidin-4-ylamino)benzo[d]oxazol-2(3H)-one;
5-(5-methyl-2-(2-morpholinopyridin-4-ylamino)pyrimidin-4-ylamino)benzo[d]oxazol-
2(3H)-one ditrifluoroacetate salt;
5-(5-methyl-2-(2-oxo-1,2,3,4-tetrahydroquinolin-6-ylamino)pyrimidin-4-
ylamino)benzo[d]oxazol-2(3H)-one;
5-(5-methyl-2-(2-oxo-1,2,3,4-tetrahydroquinolin-7-ylamino)pyrimidin-4-
ylamino)benzo[d]oxazol-2(3H)-one;
5-(5-methyl-2-(2-oxo-2,3,4,5-tetrahydro-1H-benzo[b]azepin-7-ylamino)pyrimidin-4-
ylamino)benzo[d]oxazol-2(3H)-one;
5-(5-methyl-2-(2-oxo-2,3,4,5-tetrahydro-1H-benzo[b]azepin-8-ylamino)pyrimidin-4-
ylamino)benzo[d]oxazol-2(3H)-one;
5-(5-methyl-2-(2-oxo-2,3-dihydro-1H-benzo[d]imidazol-5-ylamino)pyrimidin-4-
ylamino)benzo[d]oxazol-2(3H)-one;
5-(5-methyl-2-(3-(1-(methylamino)butyl)-5-(trifluoromethyl)phenylamino)pyrimidin-4-
ylamino)benzo[d]oxazol-2(3H)-one;
5-(5-methyl-2-(3-(1-(methylamino)ethyl)-5-(trifluoromethyl)phenylamino)pyrimidin-4-
ylamino)benzo[d]oxazol-2(3H)-one;
5-(5-methyl-2-(3-(1-(piperidin-1-yl)ethyl)phenylamino)pyrimidin-4-
ylamino)benzo[d]oxazol-2(3H)-one;
5-(5-methyl-2-(3-(1-(propylamino)ethyl)phenylamino)pyrimidin-4-
ylamino)benzo[d]oxazol-2(3H)-one;
5-(5-methyl-2-(3-(1-(pyrrolidin-1-yl)ethyl)-5-(trifluoromethyl)phenylamino)pyrimidin-4-
ylamino)benzo[d]oxazol-2(3H)-one;
5-(5-methyl-2-(3-(1-(pyrrolidin-1-yl)ethyl)phenylamino)pyrimidin-4-
ylamino)benzo[d]oxazol-2(3H)-one;
5-(5-methyl-2-(3-(1-morpholinoethyl)phenylamino)pyrimidin-4-ylamino)benzo[d]oxazol-
2(3H)-one;
5-(5-methyl-2-(3-(morpholine-4-carbonyl)phenylamino)pyrimidin-4-
ylamino)benzo[d]oxazol-2(3H)-one;
5-(5-methyl-2-(3-(pyrrolidine-1-carbonyl)phenylamino)pyrimidin-4-
ylamino)benzo[d]oxazol-2(3H)-one;
5-(5-methyl-2-(3,5-dimethyl-4-d₃-methyl)phenylamino)pyrimidin-4-
ylamino)benzo[d]oxazol-2(3H)-one;
5-(5-methyl-2-(3-methyl-4-(pyridin-3-yl)phenylamino)pyrimidin-4-
ylamino)benzo[d]oxazol-2(3H)-one formate salt;
5-(5-methyl-2-(3-methyl-4-(pyridin-4-yl)phenylamino)pyrimidin-4-
ylamino)benzo[d]oxazol-2(3H)-one formate salt;
5-(5-methyl-2-(4-(1-(pyrrolidin-1-yl)ethyl)phenylamino)pyrimidin-4-
ylamino)benzo[d]oxazol-2(3H)-one;
5-(5-methyl-2-(4-(1-morpholinoethyl)phenylamino)pyrimidin-4-ylamino)benzo[d]oxazol-
2(3H)-one;
5-(5-methyl-2-(4-(6-morpholinopyridin-3-yl)phenylamino)pyrimidin-4-
ylamino)benzo[d]oxazol-2(3H)-one trifluoroacetate salt;
5-(5-methyl-2-(4-(morpholine-4-carbonyl)phenylamino)pyrimidin-4-
ylamino)benzo[d]oxazol-2(3H)-one;
5-(5-methyl-2-(4-(pyrrolidine-1-carbonyl)phenylamino)pyrimidin-4-
ylamino)benzo[d]oxazol-2(3H)-one;
5-(5-methyl-2-(4-methyl-3-(methylsulfonyl)phenylamino)pyrimidin-4-
ylamino)benzo[d]oxazol-2(3H)-one;
5-(5-methyl-2-(4-methyl-3-(pyridin-3-yl)phenylamino)pyrimidin-4-
ylamino)benzo[d]oxazol-2(3H)-one;
5-(5-methyl-2-(4-methyl-3-(pyridin-4-yl)phenylamino)pyrimidin-4-
ylamino)benzo[d]oxazol-2(3H)-one;
5-(5-methyl-2-(5-methyl-6-(8-(2,2,2-trifluoroacetyl)-3,8-diaza-bicyclo[3.2.1]octan-3-
yl)pyridin-3-ylamino)pyrimidin-4-ylamino)benzo[d]oxazol-2(3H)-one;
5-(5-methyl-2-(5-methyl-6-(8-(2,2,2-trifluoroacetyl)-8-aza-bicyclo[3.2.1]octan-3-
yl)pyridin-3-ylamino)pyrimidin-4-ylamino)benzo[d]oxazol-2(3H)-one;
5-(5-methyl-2-(5-methyl-6-(8-acetyl)-3,8-diaza-bicyclo[3.2.1]octan-3-yl)pyridin-3-
ylamino)pyrimidin-4-ylamino)benzo[d]oxazol-2(3H)-one;
5-(5-methyl-2-(5-methyl-6-(8-methyl-3,8-diaza-bicyclo[3.2.1]octan-3-yl)pyridin-3-
ylamino)pyrimidin-4-ylamino)benzo[d]oxazol-2(3H)-one;
5-(5-methyl-2-(5-methylpyridin-2-ylamino)pyrimidin-4-ylamino)benzo[d]oxazol-2(3H)-
one;
5-(5-methyl-2-(6-(3-morpholinopyrrolidin-1-yl)pyridin-3-ylamino)pyrimidin-4-
ylamino)benzo[d]oxazol-2(3H)-one;
5-(5-methyl-2-(6-(pyrrolidin-1-yl)pyridin-3-ylamino)pyrimidin-4-ylamino)benzo[d]oxazol-
2(3H)-one;
5-(5-methyl-2-(6-methylpyridin-2-ylamino)pyrimidin-4-ylamino)benzo[d]oxazol-2(3H)-
one;
5-(5-methyl-2-(pyridin-4-ylamino)pyrimidin-4-ylamino)benzo[d]oxazol-2(3H)-one
ditrifluoroacetate salt;
5-(5-Methyl-2-m-tolylamino-pyrimidin-4-ylamino)-3H-benzooxazol-2-one;
5-(5-Methyl-2-phenylamino-pyrimidin-4-ylamino)-3H-benzooxazol-2-one;
5-(5-nitro-2-(3,4,5-trimethylphenylamino)pyrimidin-4-ylamino)benzo[d]oxazol-2(3H)-one
trifluoroacetate salt;
5-(5-nitro-2-(phenylamino)pyrimidin-4-ylamino)benzo[d]oxazol-2(3H)-one trifluoroacetate
salt;
5,5′-(5-methylpyrimidine-2,4-diyl)bis(azanediyl)dibenzo[d]oxazol-2(3H)-one;
5-[2-(2,2-Difluoro-benzo[1,3]dioxol-4-ylamino)-5-methyl-pyrimidin-4-ylamino]-3H-
benzooxazol-2-one;
5-[2-(2,2-Dimethyl-benzo[1,3]dioxol-5-ylamino)-5-methyl-pyrimidin-4-ylamino]-3H-
benzooxazol-2-one;
5-[2-(2,2-Dioxo-1H-benzo[e][1,3,4]oxathiazin-7-ylamino)-5-methyl-pyrimidin-4-ylamino]-
3H-benzooxazol-2-one;
5-[2-(2,3-Difluoro-phenylamino)-5-methyl-pyrimidin-4-ylamino]-3H-benzooxazol-2-one;
5-[2-(2,4-Difluoro-3-methoxy-phenylamino)-5-methyl-pyrimidin-4-ylamino]-3H-
benzooxazol-2-one;
5-[2-(2,4-Difluoro-5-methoxy-phenylamino)-5-methyl-pyrimidin-4-ylamino]-3H-
benzooxazol-2-one;
5-[2-(2,4-Difluoro-phenylamino)-5-methyl-pyrimidin-4-ylamino]-3H-benzooxazol-2-one;
5-[2-(2,5-Difluoro-phenylamino)-5-methyl-pyrimidin-4-ylamino]-3H-benzooxazol-2-one;
5-[2-(2-Acetyl-2,3-dihydro-1H-isoindol-5-ylamino)-5-methyl-pyrimidin-4-ylamino]-3H-
benzooxazol-2-one;
5-[2-(2-Fluoro-3,4-dimethoxy-phenylamino)-5-methyl-pyrimidin-4-ylamino]-3H-
benzooxazol-2-one;
5-[2-(2-Fluoro-3-methoxy-4-methyl-phenylamino)-5-methyl-pyrimidin-4-ylamino]-3H-
benzooxazol-2-one trifluoroacetate salt;
5-[2-(2-Fluoro-3-methoxy-phenylamino)-5-methyl-pyrimidin-4-ylamino]-3H-benzooxazol-
2-one;
5-[2-(2-Fluoro-phenylamino)-5-methyl-pyrimidin-4-ylamino]-3H-benzooxazol-2-one;
5-[2-(2-Methanesulfonyl-2,3-dihydro-1H-isoindol-5-ylamino)-5-methyl-pyrimidin-4-
ylamino]-3H-benzooxazol-2-one;
5-[2-(2-Methoxy-5-trifluoromethyl-phenylamino)-5-methyl-pyrimidin-4-ylamino]-3H-
benzooxazol-2-one;
5-[2-(3,4-Dimethyl-phenylamino)-5-methyl-pyrimidin-4-ylamino]-3H-benzooxazol-2-one;
5-[2-(3,5-Bis-trifluoromethyl-phenylamino)-5-methyl-pyrimidin-4-ylamino]-3H-
benzooxazol-2-one;
5-[2-(3,5-Dichloro-phenylamino)-5-methyl-pyrimidin-4-ylamino]-3H-benzooxazol-2-one;
5-[2-(3-Acetyl-phenylamino)-5-methyl-pyrimidin-4-ylamino]-3H-benzooxazol-2-one;
5-[2-(3-Amino-4-methoxy-phenylamino)-5-methyl-pyrimidin-4-ylamino]-3H-benzooxazol-
2-one;
5-[2-(3-Bromo-5-chloro-phenylamino)-5-methyl-pyrimidin-4-ylamino]-3H-benzooxazol-2-
one;
5-[2-(3-Bromo-5-trifluoromethyl-phenylamino)-5-methyl-pyrimidin-4-ylamino]-3H-
benzooxazol-2-one;
5-[2-(3-Bromo-phenylamino)-5-methyl-pyrimidin-4-ylamino]-3H-benzooxazol-2-one;
5-[2-(3-Chloro-4-d₃-methoxy-phenylamino)-5-methyl-pyrimidin-4-ylamino]-3H-
benzooxazol-2-one;
5-[2-(3-Chloro-4-ethoxy-phenylamino)-5-methyl-pyrimidin-4-ylamino]-3H-benzooxazol-2-
one;
5-[2-(3-Chloro-4-hydroxy-phenylamino)-5-methyl-pyrimidin-4-ylamino]-3H-benzooxazol-
2-one;
5-[2-(3-Chloro-4-methoxy-phenylamino)-5-methyl-pyrimidin-4-ylamino]-3H-benzooxazol-
2-one;
5-[2-(3-Chloro-4-trifluoromethyl-phenylamino)-5-methyl-pyrimidin-4-ylamino]-3H-
benzooxazol-2-one;
5-[2-(3-Chloro-4-trifluoromethyl-phenylamino)-5-methyl-pyrimidin-4-ylamino]-3H-
benzooxazol-2-one trifluoroacetic acid salt;
5-[2-(3-Chloro-5-fluoro-phenylamino)-5-methyl-pyrimidin-4-ylamino]-3H-benzooxazol-2-
one;
5-[2-(3-Chloro-5-trifluoromethyl-phenylamino)-5-methyl-pyrimidin-4-ylamino]-3H-
benzooxazol-2-one;
5-[2-(3-Chloro-phenylamino)-5-methyl-pyrimidin-4-ylamino]-3H-benzooxazol-2-one;
5-[2-(3-Cyclopentanesulfonyl-phenylamino)-5-methyl-pyrimidin-4-ylamino]-3H-
benzooxazol-2-one;
5-[2-(3-Dimethylamino-4-methyl-phenylamino)-5-methyl-pyrimidin-4-ylamino]-3H-
benzooxazol-2-one;
5-[2-(3-Dimethylamino-phenylamino)-5-methyl-pyrimidin-4-ylamino]-3H-benzooxazol-2-
one trifluoroacetate salt;
5-[2-(3-Ethynyl-phenylamino)-5-methyl-pyrimidin-4-ylamino]-3H-benzooxazol-2-one;
5-[2-(3-Fluoro-5-methyl-phenylamino)-5-methyl-pyrimidin-4-ylamino]-3H-benzooxazol-2-
one;
5-[2-(3-Fluoro-5-trifluoromethyl-phenylamino)-5-methyl-pyrimidin-4-ylamino]-3H-
benzooxazol-2-one triflouroacetate salt;
5-[2-(4-Acetyl-phenylamino)-5-methyl-pyrimidin-4-ylamino]-3H-benzooxazol-2-one;
5-[2-(4-Bromo-2-fluoro-3-methoxy-phenylamino)-5-methyl-pyrimidin-4-ylamino]-3H-
benzooxazol-2-one;
5-[2-(4-Bromo-2-fluoro-phenylamino)-5-methyl-pyrimidin-4-ylamino]-3H-benzooxazol-2-
one;
5-[2-(4-Bromo-3-trifluoromethyl-phenylamino)-5-methyl-pyrimidin-4-ylamino]-3H-
benzooxazol-2-one;
5-[2-(4-Chloro-2,5-dimethyl-phenylamino)-5-methyl-pyrimidin-4-ylamino]-3H-
benzooxazol-2-one;
5-[2-(4-Chloro-3-ethoxy-phenylamino)-5-methyl-pyrimidin-4-ylamino]-3H-benzooxazol-2-
one;
5-[2-(4-Chloro-3-methoxy-phenylamino)-5-methyl-pyrimidin-4-ylamino]-3H-benzooxazol-
2-one;
5-[2-(4-Chloro-3-trifluoromethoxy-phenylamino)-5-methyl-pyrimidin-4-ylamino]-3H-
benzooxazol-2-one;
5-[2-(4-Chloro-3-trifluoromethyl-phenylamino)-5-methyl-pyrimidin-4-ylamino]-3H-
benzooxazol-2-one;
5-[2-(4-Chloro-phenylamino)-5-methyl-pyrimidin-4-ylamino]-3H-benzooxazol-2-one;
5-[2-(4-Cyclobutylmethoxy-phenylamino)-5-methyl-pyrimidin-4-ylamino]-3H-
benzooxazol-2-one;
5-[2-(4-Cyclohexylmethoxy-3-trifluoromethyl-phenylamino)-5-methyl-pyrimidin-4-
ylamino]-3H-benzooxazol-2-one;
5-[2-(4-d₃-Methoxy-phenylamino)-5-methyl-pyrimidin-4-ylamino]-3H-benzooxazol-2-one;
5-[2-(4-Dimethylamino-phenylamino)-5-methyl-pyrimidin-4-ylamino]-3H-benzooxazol-2-
one;
5-[2-(4-Ethoxy-3-trifluoromethyl-phenylamino)-5-methyl-pyrimidin-4-ylamino]-3H-
benzooxazol-2-one;
5-[2-(4-Ethyl-2-fluoro-3-methoxy-phenylamino)-5-methyl-pyrimidin-4-ylamino]-3H-
benzooxazol-2-one trifluoroacetate salt;
5-[2-(4-Fluoro-3-methoxy-phenylamino)-5-methyl-pyrimidin-4-ylamino]-3H-benzooxazol-
2-one;
5-[2-(4-Fluoro-3-trifluoromethoxy-phenylamino)-5-methyl-pyrimidin-4-ylamino]-3H-
benzooxazol-2-one;
5-[2-(4-Fluoro-phenylamino)-5-methyl-pyrimidin-4-ylamino]-3H-benzooxazol-2-one;
5-[2-(4-Hydroxy-naphthalen-2-ylamino)-5-methyl-pyrimidin-4-ylamino]-3H-benzooxazol-
2-one;
5-[2-(4-Hydroxy-phenylamino)-5-methyl-pyrimidin-4-ylamino]-3H-benzooxazol-2-one;
5-[2-(4-Iodo-3,5-dimethyl-phenylamino)-5-methyl-pyrimidin-4-ylamino]-3H-benzooxazol-
2-one;
5-[2-(4-Isobutoxy-phenylamino)-5-methyl-pyrimidin-4-ylamino]-3H-benzooxazol-2-one;
5-[2-(4-Isopropyl-3-methyl-phenylamino)-5-methyl-pyrimidin-4-ylamino]-3H-
benzooxazol-2-one;
5-[2-(4-Methoxy-naphthalen-2-ylamino)-5-methyl-pyrimidin-4-ylamino]-3H-benzooxazol-
2-one;
5-[2-(6-Methanesulfonyl-pyridin-3-ylamino)-5-methyl-pyrimidin-4-ylamino]-3H-
benzooxazol-2-one;
5-[2-(9-Isopropylamino-6,7,8,9-tetrahydro-5H-benzocyclohepten-2-ylamino)-5-methyl-
pyrimidin-4-ylamino]-3H-benzooxazol-2-one;
5-[2-(Isoquinolin-6-ylamino)-5-methyl-pyrimidin-4-ylamino]-3H-benzooxazol-2-one;
5-[2-(Isoquinolin-7-ylamino)-5-methyl-pyrimidin-4-ylamino]-3H-benzooxazol-2-one;
5-[5-Ethynyl-2-(3,4,5-trimethyl-phenylamino)-pyrimidin-4-ylamino]-3H-benzooxazol-2-
one trifluoroacetate salt;
5-[5-Hydroxymethyl-2-(3,4,5-trimethyl-phenylamino)-pyrimidin-4-ylamino]-3H-
benzooxazol-2-one;
5-[5-Methyl-2-(2,3,4,5-tetrafluoro-phenylamino)-pyrimidin-4-ylamino]-3H-benzooxazol-2-
one;
5-[5-Methyl-2-(2,3,5-trifluoro-phenylamino)-pyrimidin-4-ylamino]-3H-benzooxazol-2-one;
5-[5-Methyl-2-(2,4,5-trifluoro-phenylamino)-pyrimidin-4-ylamino]-3H-benzooxazol-2-one;
5-[5-Methyl-2-(2,4,6-trifluoro-phenylamino)-pyrimidin-4-ylamino]-3H-benzooxazol-2-one;
5-[5-Methyl-2-(2,4,6-trimethyl-phenylamino)-pyrimidin-4-ylamino]-3H-benzooxazol-2-
one;
5-[5-Methyl-2-(2-methyl-3-trifluoromethyl-phenylamino)-pyrimidin-4-ylamino]-3H-
benzooxazol-2-one;
5-[5-Methyl-2-(3,5-dimethyl-4-d3-methyl-phenylamino)-pyrimidin-4-ylamino]-3H-
benzooxazol-2-one trifluoroacetate salt;
5-[5-Methyl-2-(3-trifluoromethyl-phenylamino)-pyrimidin-4-ylamino]-3H-benzooxazol-2-
one;
5-[5-Methyl-2-(3-vinyl-phenylamino)-pyrimidin-4-ylamino]-3H-benzooxazol-2-one
trifluoroacetate salt;
5-[5-Methyl-2-(4-methyl-3-trifluoromethyl-phenylamino)-pyrimidin-4-ylamino]-3H-
benzooxazol-2-one;
5-[5-Methyl-2-(4-methyl-3-trifluoromethyl-phenylamino)-pyrimidin-4-ylamino]-3H-
benzooxazol-2-one trifluoracetic acid salt;
5-[5-Methyl-2-(4-methylsulfanyl-3-trifluoromethyl-phenylamino)-pyrimidin-4-ylamino]-
3H-benzooxazol-2-one;
5-[5-Methyl-2-(4-trifluoromethoxy-3-trifluoromethyl-phenylamino)-pyrimidin-4-ylamino]-
3H-benzooxazol-2-one;
5-[5-Methyl-2-(4-trifluoromethyl-phenylamino)-pyrimidin-4-ylamino]-3H-benzooxazol-2-
one;
5-[5-Methyl-2-(methyl-phenyl-amino)-pyrimidin-4-ylamino]-3H-benzooxazol-2-one;
5-[5-Methyl-2-(naphthalen-2-ylamino)-pyrimidin-4-ylamino]-3H-benzooxazol-2-one;
5-[5-Methyl-2-(pyridin-3-ylamino)-pyrimidin-4-ylamino]-3H-benzooxazol-2-one;
5-[5-Methyl-4-(2-oxo-2,3-dihydro-benzooxazol-5-ylamino)-pyrimidin-2-ylamino]-pyridine-
2-carboxylic acid cyclobutylamide;
5-{2-[2-Fluoro-3-(2-methoxy-ethoxy)-4-methyl-phenylamino]-5-methyl-pyrimidin-4-
ylamino}-3H-benzooxazol-2-one;
5-{2-[3-Chloro-4-(2-diethylamino-ethoxy)-phenylamino]-5-methyl-pyrimidin-4-ylamino}-
3H-benzooxazol-2-one;
5-{2-[3-Chloro-4-(2-morpholin-4-yl-ethoxy)-phenylamino]-5-methyl-pyrimidin-4-
ylamino}-3H-benzooxazol-2-one;
5-{2-[4-(2-Diethylamino-ethoxy)-phenylamino]-5-methyl-pyrimidin-4-ylamino}-3H-
benzooxazol-2-one;
5-{2-[4-(2-Dimethylamino-ethoxy)-phenylamino]-5-methyl-pyrimidin-4-ylamino}-3H-
benzooxazol-2-one;
5-{2-[4-(2-Methoxy-ethoxy)-3-trifluoromethyl-phenylamino]-5-methyl-pyrimidin-4-
ylamino}-3H-benzooxazol-2-one;
5-{2-[4-(3-Diethylamino-pyrrolidine-1-carbonyl)-phenylamino]-5-methyl-pyrimidin-4-
ylamino}-3H-benzooxazol-2-one;
5-{2-[4-(3-Dimethylamino-propoxy)-3-trifluoromethyl-phenylamino]-5-methyl-pyrimidin-
4-ylamino}-3H-benzooxazol-2-one;
5-{2-[5-Fluoro-6-(hexahydro-pyrrolo[3,4-c]pyrrol-2-yl)-pyridin-3-ylamino]-5-methyl-
pyrimidin-4-ylamino}-3H-benzooxazol-2-one;
5-{2-[6-(2,5-Diaza-bicyclo[2.2.1]hept-2-yl)-5-fluoro-pyridin-3-ylamino]-5-methyl-
pyrimidin-4-ylamino}-3H-benzooxazol-2-one;
5-{2-[6-(4-Cyclopropylmethyl-piperazine-1-carbonyl)-pyridin-3-ylamino]-5-methyl-
pyrimidin-4-ylamino}-3H-benzooxazol-2-one;
5-{2-[6-(4-Isobutyl-piperazine-1-carbonyl)-pyridin-3-ylamino]-5-methyl-pyrimidin-4-
ylamino}-3H-benzooxazol-2-one;
5-{2-[6-(5-Cyclopropanecarbonyl-hexahydro-pyrrolo[3,4-c]pyrrol-2-yl)-5-fluoro-pyridin-3-
ylamino]-5-methyl-pyrimidin-4-ylamino}-3H-benzooxazol-2-one;
5-{2-[6-(5-Cyclopropylmethyl-2,5-diaza-bicyclo[2.2.1]hept-2-yl)-5-fluoro-pyridin-3-
ylamino]-5-methyl-pyrimidin-4-ylamino}-3H-benzooxazol-2-one;
5-{2-[6-(5-Cyclopropylmethyl-hexahydro-pyrrolo[3,4-c]pyrrol-2-yl)-5-fluoro-pyridin-3-
ylamino]-5-methyl-pyrimidin-4-ylamino}-3H-benzooxazol-2-one;
5-{2-[9-(3-Diethylamino-pyrrolidin-1-yl)-6,7,8,9-tetrahydro-5H-benzocyclohepten-2-
ylamino]-5-methyl-pyrimidin-4-ylamino}-3H-benzooxazol-2-one;
5-{3-Fluoro-5-[5-methyl-4-(2-oxo-2,3-dihydro-benzooxazol-5-ylamino)-pyrimidin-2-
ylamino]-pyridin-2-yl}-hexahydro-pyrrolo[3,4-c]pyrrole-2-carboxylic acid tert-butyl ester;
5-{3-Fluoro-5-[5-methyl-4-(2-oxo-2,3-dihydro-benzooxazol-5-ylamino)-pyrimidin-2-
ylamino]-pyridin-2-yl}-2,5-diaza-bicyclo[2.2.1]heptane-2-carboxylic acid tert-butyl ester;
5-{5-Methyl-2-[4-(2-morpholin-4-yl-ethoxy)-phenylamino]-pyrimidin-4-ylamino}-3H-
benzooxazol-2-one;
5-{5-Methyl-2-[4-(3-methyl-butoxy)-phenylamino]-pyrimidin-4-ylamino}-3H-
benzooxazol-2-one;
5-{5-Methyl-2-[6-((S)-3-methyl-piperazin-1-yl)-pyridin-3-ylamino]-pyrimidin-4-ylamino}-
3H-benzooxazol-2-one;
5-{5-Methyl-2-[6-(piperazine-1-carbonyl)-pyridin-3-ylamino]-pyrimidin-4-ylamino}-3H-
benzooxazol-2-one;
5-fluoro-N2-(3,4,5-trimethyl)phenyl-N4-(2-oxo-2,3-dihydro-1,3-benzoxazol-5-yl)-2,4-
pyrimidinediamine;
5-fluoro-N2-(3-methoxy-5-trifluoromethyl)phenyl-N4-(2-oxo-2,3-dihydro-1,3-benzoxazol-
5-yl)-2,4-pyrimidinediamine;
5-fluoro-N2-(4-methoxy-3-trifluoromethyl)phenyl-N4-(2-oxo-2,3-dihydro-1,3-benzoxazol-
5-yl)-2,4-pyrimidinediamine;
5-fluoro-N2-[3-(1-hydroxy-2,2,2-trifluoroethyl)]phenyl-N4-(2-oxo-2,3-dihydro-1,3-
benzoxazol-5-yl)-2,4-pyrimidinediamine;
5-methyl-N2-(1-methylindazol-5-yl)-N4-(2-oxo-2,3-dihydro-1,3-benzoxazol-5-yl)-2,4-
pyrimidinediamine;
5-methyl-N2-(1-methylindazol-6-yl)-N4-(2-oxo-2,3-dihydro-1,3-benzoxazol-5-yl)-2,4-
pyrimidinediamine;
5-methyl-N2-(2-methyl-3-methylaminocarbonylmethoxy)phenyl-N4-(2-oxo-2,3-dihydro-
1,3-benzoxazol-5-yl)-2,4-pyrimidinediamine;
5-methyl-N2-(2-methylpyridin-4-yl)-N4-(2-oxo-2,3-dihydro-1,3-benzoxazol-5-yl)-2,4-
pyrimidinediamine;
5-methyl-N2-(3,4-methylenedioxy)phenyl-N4-(2-oxo-2,3-dihydro-1,3-benzoxazol-5-yl)-
2,4-pyrimidinediamine;
5-methyl-N2-(3-methyl-5-trifluoromethoxy)phenyl-N4-(2-oxo-2,3-dihydro-1,3-benzoxazol-
5-yl)-2,4-pyrimidinediamine;
5-methyl-N2-(3-methyl-5-trifluoromethyl)phenyl-N4-(2-oxo-2,3-dihydro-1,3-benzoxazol-
5-yl)-2,4-pyrimidinediamine;
5-methyl-N2-(3-methylaminocarbonylmethoxy)phenyl-N4-(2-oxo-2,3-dihydro-1,3-
benzoxazol-5-yl)-2,4-pyrimidinediamine;
5-methyl-N2-(3-methylindazol-5-yl)-N4-(2-oxo-2,3-dihydro-1,3-benzoxazol-5-yl)-2,4-
pyrimidinediamine;
5-methyl-N2-(3-methylindazol-6-yl)-N4-(2-oxo-2,3-dihydro-1,3-benzoxazol-5-yl)-2,4-
pyrimidinediamine;
5-methyl-N2-(3-methylisoxazolo[5,4-b]pyridin-5-yl)-N4-(2-oxo-2,3-dihydro-1,3-
benzoxazol-5-yl)-2,4-pyrimidinediamine;
5-methyl-N2-(4-morpholino)phenyl-N4-(2-oxo-2,3-dihydro-1,3-benzoxazol-5-yl)-2,4-
pyrimidinediamine;
5-methyl-N2-(4-morpholinomethyl)phenyl-N4-(2-oxo-2,3-dihydro-1,3-benzoxazol-5-yl)-
2,4-pyrimidinediamine;
5-methyl-N2-[2-(2R-methylmorpholino)pyridin-5-yl]-N4-(2-oxo-2,3-dihydro-1,3-
benzoxazol-5-yl)-2,4-pyrimidinediamine;
5-methyl-N2-[2-(2S-methylmorpholino)pyridin-5-yl]-N4-(2-oxo-2,3-dihydro-1,3-
benzoxazol-5-yl)-2,4-pyrimidinediamine;
5-methyl-N2-[2-(3-methyl-3,7-diazabicyclo[3.3.0]octan-7-yl)pyridin-5-yl]-N4-(2-oxo-2,3-
dihydro-1,3-benzoxazol-5-yl)-2,4-pyrimidinediamine;
5-methyl-N2-[2-(3S-methylmorpholino)pyridin-5-yl]-N4-(2-oxo-2,3-dihydro-1,3-
benzoxazol-5-yl)-2,4-pyrimidinediamine;
5-methyl-N2-[2-(7-methyl-2,7-diazaspiro[4.4]nonan-2-yl)pyridin-5-yl]-N4-(2-oxo-2,3-
dihydro-1,3-benzoxazol-5-yl)-2,4-pyrimidinediamine;
5-methyl-N2-[2-(octahydroisoindol-1-yl)pyridin-5-yl]-N4-(2-oxo-2,3-dihydro-1,3-
benzoxazol-5-yl)-2,4-pyrimidinediamine;
5-methyl-N2-[3-(1,3-oxazol-5-yl)]phenyl-N4-(2-oxo-2,3-dihydro-1,3-benzoxazol-5-yl)-2,4-
pyrimidinediamine;
5-methyl-N2-[3-(4-methylpiperazin-1-yl)carbonyl]phenyl-N4-(2-oxo-2,3-dihydro-1,3-
benzoxazol-5-yl)-2,4-pyrimidinediamine;
5-methyl-N2-[3-(morpholinocarbonylmethoxy)]phenyl-N4-(2-oxo-2,3-dihydro-1,3-
benzoxazol-5-yl)-2,4-pyrimidinediamine;
5-methyl-N2-[3-methylaminocarbonyl-4-(4-methylpiperazino)]phenyl-N4-(2-oxo-2,3-
dihydro-1,3-benzoxazol-5-yl)-2,4-pyrimidinediamine;
5-methyl-N2-[4-(4-methylpiperazin-1-yl)carbonyl]phenyl-N4-(2-oxo-2,3-dihydro-1,3-
benzoxazol-5-yl)-2,4-pyrimidinediamine;
5-methyl-N2-{2-[(1R,4R)-2-oxa-5-azabicyclo[2.2.1]heptan-5-yl]pyridin-5-yl}-N4-(2-oxo-
2,3-dihydro-1,3-benzoxazol-5-yl)-2,4-pyrimidinediamine;
5-methyl-N4-(2-oxo-2,3-dihydro-1,3-benzoxazol-5-yl)-N2-(1-oxo-1,3-dihydro-
isobenzofuran-6-yl)-2,4-pyrimidinediamine;
5-methyl-N4-(2-oxo-2,3-dihydro-1,3-benzoxazol-5-yl)-N2-(1-oxo-1,3-dihydro-
isobenzofuran-5-yl)-2,4-pyrimidinediamine;
5-methyl-N4-(2-oxo-2,3-dihydro-1,3-benzoxazol-5-yl)-N2-(1-oxo-1,3-dihydro-2H-isoindol-
6-yl)-2,4-pyrimidinediamine;
5-methyl-N4-(2-oxo-2,3-dihydro-1,3-benzoxazol-5-yl)-N2-(1-oxo-1,3-dihydro-7-
methylisobenzofuran-4-yl)-2,4-pyrimidinediamine;
5-methyl-N4-(2-oxo-2,3-dihydro-1,3-benzoxazol-5-yl)-N2-(1-oxo-1,3-dihydro-4-
methylisobenzofuran-6-yl)-2,4-pyrimidinediamine;
5-methyl-N4-(2-oxo-2,3-dihydro-1,3-benzoxazol-5-yl)-N2-(2H-pyrido[3,2-b][1,4]oxazin-
3(4H)-one-7-yl)-2,4-pyrimidinediamine;
5-methyl-N4-(2-oxo-2,3-dihydro-1,3-benzoxazol-5-yl)-N2-(2H-pyrido[3,2-b][1,4]oxazin-
3(4H)-one-6-yl)-2,4-pyrimidinediamine;
5-methyl-N4-(2-oxo-2,3-dihydro-1,3-benzoxazol-5-yl)-N2-(3,4,5-trimethyl)phenyl-2,4-
pyrimidinediamine benzenesulfonic acid salt;
5-methyl-N4-(2-oxo-2,3-dihydro-1,3-benzoxazol-5-yl)-N2-(3,4,5-trimethyl)phenyl-2,4-
pyrimidinediamine pamoic acid salt;
5-methyl-N4-(2-oxo-2,3-dihydro-1,3-benzoxazol-5-yl)-N2-(3,4,5-trimethyl)phenyl-2,4-
pyrimidinediamine methane sulfonic acid salt;
5-methyl-N4-(2-oxo-2,3-dihydro-1,3-benzoxazol-5-yl)-N2-(3,4,5-trimethyl)phenyl-2,4-
pyrimidinediamine hydrochloric acid salt;
5-methyl-N4-(2-oxo-2,3-dihydro-1,3-benzoxazol-5-yl)-N2-(3,4,5-trifluoro)phenyl-2,4-
pyrimidinediamine;
5-methyl-N4-(2-oxo-2,3-dihydro-1,3-benzoxazol-5-yl)-N2-(3,4,5-trimethyl)phenyl-2,4-
pyrimidinediamine sodium salt;
5-methyl-N4-(2-oxo-2,3-dihydro-1,3-benzoxazol-5-yl)-N2-(4-
phenylcarbonylamino)phenyl-2,4-pyrimidinediamine;
5-methyl-N4-(2-oxo-2,3-dihydro-1,3-benzoxazol-5-yl)-N2-(4-piperazino)phenyl-2,4-
pyrimidinediamine;
5-methyl-N4-(2-oxo-2,3-dihydro-1,3-benzoxazol-5-yl)-N2-[2-(1,3,5-trimethyl-3,7-
diazabicyclo[3.3.1]nonan-7-yl)pyridin-5-yl]-2,4-pyrimidinediamine;
5-methyl-N4-(2-oxo-2,3-dihydro-1,3-benzoxazol-5-yl)-N2-[2-(cis-3,4,5-
trimethylpiperazino)pyridin-5-yl]-2,4-pyrimidinediamine;
5-methyl-N4-(2-oxo-2,3-dihydro-1,3-benzoxazol-5-yl)-N2-[2-(trans-2,4,5-
trimethylpiperazino)pyridin-5-yl]-2,4-pyrimidinediamine;
5-methyl-N4-(2-oxo-2,3-dihydro-1,3-benzoxazol-5-yl)-N2-[3-(4-propylpiperazino)-4-
trifluoromethyl]phenyl-2,4-pyrimidinediamine;
5-methyl-N4-(2-oxo-2,3-dihydro-1,3-benzoxazol-5-yl)-N2-[3-(pyridin-4-yl)]phenyl-2,4-
pyrimidinediamine;
5-methyl-N4-(2-oxo-2,3-dihydro-1,3-benzoxazol-5-yl)-N2-[3-(pyridin-3-yl)]phenyl-2,4-
pyrimidinediamine;
5-methyl-N4-(2-oxo-2,3-dihydro-1,3-benzoxazol-5-yl)-N2-[4-(pyridin-3-yl)]phenyl-2,4-
pyrimidinediamine;
5-methyl-N4-(2-oxo-2,3-dihydro-1,3-benzoxazol-5-yl)-N2-[4-(pyridin-4-yl)-3-
trifluoromethyl]phenyl-2,4-pyrimidinediamine;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine arginine salt;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine Tris salt;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine dipotassium salt;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine lysine salt;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine bis-sodium salt;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine calcium salt;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine magnesium salt;
5-methyl-N4-[3-(phosphonooxy)methyl-2-oxo-2,3-dihydro-1,3-benzoxazol-5-yl]-N2-
(3,4,5-trimethyl)phenyl-2,4-pyrimidinediamine bis-choline salt;
6-(4-(2,3-dihydro-2-oxobenzo[d]oxazol-5-ylamino)-5-fluoropyrimidin-2-ylamino)-3,4-
dihydroquinolin-2(1H)-one trifluoroacetate salt;
6-(5-methyl-2-(6-(4-methylpiperazin-1-yl)pyridin-3-ylamino)pyrimidin-4-
ylamino)benzo[d]oxazol-2(3H)-one;
6-(5-methyl-4-(2-oxo-2,3-dihydrobenzo[d]oxazol-5-ylamino)pyrimidin-2-ylamino)-2H-
benzo[b][1,4]oxazin-3(4H)-one;
6-[5-Methyl-4-(2-oxo-2,3-dihydro-benzooxazol-5-ylamino)-pyrimidin-2-ylamino]-3,4-
dihydro-2H-isoquinolin-1-one;
7-((diethylamino)methyl)-5-(2-(3,4,5-trimethylphenylamino)-5-methylpyrimidin-4-
ylamino)benzo[d]oxazol-2(3H)-one ditrifluoroacetate salt;
7-(4-(2,3-dihydro-2-oxobenzo[d]oxazol-5-ylamino)-5-fluoropyrimidin-2-ylamino)-2H-
benzo[b][1,4]oxazin-3(4H)-one formate salt;
7-(4-(2,3-dihydro-2-oxobenzo[d]oxazol-5-ylamino)-5-fluoropyrimidin-2-ylamino)-4,5-
dihydro-1H-benzo[b]azepin-2(3H)-one trifluoroacetate salt;
7-(4-(2,3-dihydro-2-oxobenzo[d]oxazol-5-ylamino)-5-fluoropyrimidin-2-ylamino)-4,5-
dihydro-1-methyl-1H-benzo[b]azepin-2(3H)-one trifluoroacetate salt;
7-(5-methyl-4-(2-oxo-2,3-dihydrobenzo[d]oxazol-5-ylamino)pyrimidin-2-ylamino)-2H-
benzo[b][1,4]oxazin-3(4H)-one;
7-fluoro-5-(2-(3-methoxy-5-(trifluoromethyl)phenylamino)-5-methylpyrimidin-4-
ylamino)benzo[d]oxazol-2(3H)-one;
7-fluoro-5-(2-(3-methoxy-5-methylphenylamino)-5-methylpyrimidin-4-
ylamino)benzo[d]oxazol-2(3H)-one;
7-fluoro-5-(2-(4-fluoro-3-(methylsulfonyl)phenylamino)-5-methylpyrimidin-4-
ylamino)benzo[d]oxazol-2(3H)-one;
7-fluoro-5-(2-(4-fluoro-3,5-dimethylphenylamino)-5-methylpyrimidin-4-
ylamino)benzo[d]oxazol-2(3H)-one;
7-fluoro-5-(2-(4-fluoro-3-methoxy-5-methylphenylano)-5-methylpyrimidin-4-
ylamino)benzo[d]oxazol-2(3H)-one;
7-fluoro-5-(2-(4-methoxy-3-methylphenylamino)-5-methylpyrimidin-4-
ylamino)benzo[d]oxazol-2(3H)-one;
7-fluoro-5-(5-fluoro-2-(3-methoxy-4,5-dimethylphenylamino)pyrimidin-4-
ylamino)benzo[d]oxazol-2(3H)-one trifluoroacetate salt;
7-fluoro-5-(5-methyl-2-(3-(methylsulfonyl)phenylamino)pyrimidin-4-
ylamino)benzo[d]oxazol-2(3H)-one;
7-fluoro-5-(5-methyl-2-(3,4,5-trimethylphenylamino)pyrimidin-4-ylamino)benzo[d]oxazol-
2(3H)-one;
7-fluoro-5-(5-methyl-2-(6-(4-methylpiperazin-1-yl)pyridin-3-ylamino)pyrimidin-4-
ylamino)benzo[d]oxazol-2(3H)-one;
7-fluoro-5-(5-methyl-2-(6-morpholinopyridin-3-ylamino)pyrimidin-4-
ylamino)benzo[d]oxazol-2(3H)-one;
7-methyl-5-(5-methyl-2-(3-(methylsulfonyl)phenylamino)pyrimidin-4-
ylamino)benzo[d]oxazol-2(3H)-one;
7-methyl-5-(5-methyl-2-(3,4,5-trimethylphenylamino)pyrimidin-4-
ylamino)benzo[d]oxazol-2(3H)-one;
7-methyl-5-(5-methyl-2-(6-(4-methylpiperazin-1-yl)pyridin-3-ylamino)pyrimidin-4-
ylamino)benzo[d]oxazol-2(3H)-one;
7-methyl-5-(5-methyl-2-(6-morpholinopyridin-3-ylamino)pyrimidin-4-
ylamino)benzo[d]oxazol-2(3H)-one;
Disodium(5-(2-(3-methoxy-4,5-dimethylphenylamino)-5-methylpyrimidin-4-ylamino)-2-
oxobenzo[d]oxazol-3(2H)-yl)methyl phosphate;
Di-sodium(5-(2-(4-fluoro-3-trideuteromethoxy-5-methylphenylamino)-6-D-5-
trideuteromethylpyrimidin-4-ylamino)-2-oxobenzo[d]oxazol-3(2H)-yl)methyl phosphate;
di-tert-butyl(5-(2-(4-(cyclobutylcarbamoyl)phenylamino)-5-methylpyrimidin-4-ylamino)-
2-oxobenzo[d]oxazol-3(2H)-yl)methyl phosphate;
di-tert-butyl(5-(2-(4-carbamoylphenylamino)-5-methylpyrimidin-4-ylamino)-2-
oxobenzo[d]oxazol-3(2H)-yl)methyl phosphate;
ethyl4-(4-(2,3-dihydro-2-oxobenzo[d]oxazol-5-ylamino)-5-methylpyrimidin-2-ylamino)-2-
methoxy-6-methylbenzoate;
5-(2-(6-(1,4-diazabicyclo[3.2.2]nonan-4-yl)-5-chloropyridin-3-ylamino)-5-
methylpyrimidin-4-ylamino)benzo[d]oxazol-2(3H)-one;
methyl2-(4-fluoro-3,5-dimethylphenylamino)-4-(2-oxo-2,3-dihydrobenzo[d]oxazol-5-
ylamino)pyrimidine-5-carboxylate trifluoroacetate salt;
methyl3-(3-methyl-5-(5-methyl-4-(2-oxo-2,3-dihydrobenzo[d]oxazol-5-
ylamino)pyrimidin-2-ylamino)pyridin-2-yl)-8-azabicyclo[3.2.1]octane-8-carboxylate;
N-(1-(5-(5-methyl-4-(2-oxo-2,3-dihydrobenzo[d]oxazol-5-ylamino)pyrimidin-2-
ylamino)pyridin-2-yl)pyrrolidin-3-yl)acetamide;
N-(1-(5-(5-methyl-4-(2-oxo-2,3-dihydrobenzo[d]oxazol-5-ylamino)pyrimidin-2-
ylamino)pyridin-2-yl)pyrrolidin-3-yl)methanesulfonamide;
N-(1-(5-(5-methyl-4-(2-oxo-2,3-dihydrobenzo[d]oxazol-5-ylamino)pyrimidin-2-
ylamino)pyridin-2-yl)pyrrolidin-3-yl)cyclopropanecarboxamide;
N-(1-(5-(5-methyl-4-(2-oxo-2,3-dihydrobenzo[d]oxazol-5-ylamino)pyrimidin-2-
ylamino)pyridin-2-yl)pyrrolidin-3-yl)pivalamide;
N-(2-Diethylamino-ethyl)-4-[5-methyl-4-(2-oxo-2,3-dihydro-benzooxazol-5-ylamino)-
pyrimidin-2-ylamino]-benzamide;
N-(5-(5-methyl-4-(2-oxo-2,3-dihydrobenzo[d]oxazol-5-ylamino)pyrimidin-2-
ylamino)pyridin-2-yl)methanesulfonamide;
N-(5-(5-methyl-4-(3-methyl-2-oxo-2,3-dihydrobenzo[d]oxazol-5-ylamino)pyrimidin-2-
ylamino)pyridin-2-yl)methanesulfonamide;
N,N-Dimethyl-4-[5-methyl-4-(2-oxo-2,3-dihydro-benzooxazol-5-ylamino)-pyrimidin-2-
ylamino]-benzamide;
N-{2-[2-(2,2-Dimethyl-propionyl)-2,3-dihydro-1H-isoindol-5-ylamino]-5-methyl-
pyrimidin-4-yl}-N-[3-(2,2-dimethyl-propionyl)-2-oxo-2,3-dihydro-benzooxazol-5-yl]-2,2-
dimethyl-propionamide;
N-{4-[5-Methyl-4-(2-oxo-2,3-dihydro-benzooxazol-5-ylamino)-pyrimidin-2-ylamino]-2-
trifluoromethyl-phenyl}-acetamide;
N2-((3-aminosulfonyl)phenyl)-N4-(2-oxo-2,3-dihydrobenzo[d]oxazol-6-yl)-5-
methylpyrimidine-2,4-diamine;
N2-((3-aminosulfonyl)phenyl)-N4-(3-methyl-2-oxo-2,3-dihydrobenzo[d]oxazol-6-yl)-5-
fluoropyrimidine-2,4-diamine;
N2-((3-aminosulfonyl)phenyl)-N4-(3-methyl-2-oxo-2,3-dihydrobenzo[d]oxazol-6-yl)-5-
methylpyrimidine-2,4-diamine;
N2-((3-aminosulfonyl)phenyl)-N4-(3-methyl-2-oxo-2,3-dihydrobenzo[d]oxazol-5-yl)-5-
methylpyrimidine-2,4-diamine;
N2-((3-aminosulfonyl)phenyl)-N4-(3-methyl-2-oxo-2,3-dihydrobenzo[d]oxazol-5-yl)-5-
fluoropyrimidine-2,4-diamine;
N2-((3-methylsulfonyl)phenyl)-N4-(2-oxo-2,3-dihydrobenzo[d]oxazol-6-yl)-5-
methylpyrimidine-2,4-diamine;
N2-((3-methylsulfonyl)phenyl)-N4-(3-methyl-2-oxo-2,3-dihydrobenzo[d]oxazol-6-yl)-5-
fluoropyrimidine-2,4-diamine;
N2-((3-methylsulfonyl)phenyl)-N4-(3-methyl-2-oxo-2,3-dihydrobenzo[d]oxazol-6-yl)-5-
methylpyrimidine-2,4-diamine;
N2-((3-methylsulfonyl)phenyl)-N4-(3-methyl-2-oxo-2,3-dihydrobenzo[d]oxazol-5-yl)-5-
methylpyrimidine-2,4-diamine;
N2-((3-methylsulfonyl)phenyl)-N4-(3-methyl-2-oxo-2,3-dihydrobenzo[d]oxazol-5-yl)-5-
fluoropyrimidine-2,4-diamine;
N2-((3-methylsulfonylamino)phenyl)-N4-(2-oxo-2,3-dihydrobenzo[d]oxazol-6-yl)-5-
methylpyrimidine-2,4-diamine;
N2-((3-methylsulfonylamino)phenyl)-N4-(3-methyl-2-oxo-2,3-dihydrobenzo[d]oxazol-6-
yl)-5-fluoropyrimidine-2,4-diamine;
N2-((3-methylsulfonylamino)phenyl)-N4-(3-methyl-2-oxo-2,3-dihydrobenzo[d]oxazol-5-
yl)-5-methylpyrimidine-2,4-diamine;
N2-((3-methylsulfonylamino)phenyl)-N4-(3-methyl-2-oxo-2,3-dihydrobenzo[d]oxazol-6-
yl)-5-methylpyrimidine-2,4-diamine;
N2-((3-methylsulfonylamino)phenyl)-N4-(3-methyl-2-oxo-2,3-dihydrobenzo[d]oxazol-5-
yl)-5-fluoropyrimidine-2,4-diamine;
N2-((3-methylsulfonylamino-4-methyl)phenyl)-N4-(2-oxo-2,3-dihydrobenzo[d]oxazol-5-
yl)-5-methylpyrimidine-2,4-diamine;
N2-((3-methylsulfonylamino-4-methyl)phenyl)-N4-(2-oxo-2,3-dihydrobenzo[d]oxazol-5-
yl)-5-fluoropyrimidine-2,4-diamine;
N2-((3-methylsulfonylamino-4-methyl)phenyl)-N4-(2-oxo-2,3-dihydrobenzo[d]oxazol-6-
yl)-5-methylpyrimidine-2,4-diamine;
N2-((3-methylsulfonylamino-4-methyl)phenyl)-N4-(3-methyl-2-oxo-2,3-
dihydrobenzo[d]oxazol-5-yl)-5-methylpyrimidine-2,4-diamine;
N2-((3-methylsulfonylamino-4-methyl)phenyl)-N4-(3-methyl-2-oxo-2,3-
dihydrobenzo[d]oxazol-6-yl)-5-methylpyrimidine-2,4-diamine;
N2-((3-methylsulfonylamino-4-methyl)phenyl)-N4-(3-methyl-2-oxo-2,3-
dihydrobenzo[d]oxazol-5-yl)-5-fluoropyrimidine-2,4-diamine;
N2-((3-methylsulfonylamino-4-methyl)phenyl)-N4-(3-methyl-2-oxo-2,3-
dihydrobenzo[d]oxazol-6-yl)-5-fluoropyrimidine-2,4-diamine;
N2-((4-aminosulfonyl)phenyl)-N4-(2-oxo-2,3-dihydrobenzo[d]oxazol-6-yl)-5-
methylprimidine-2,4-diamine;
N2-((4-aminosulfonyl)phenyl)-N4-(3-methyl-2-oxo-2,3-dihydrobenzo[d]oxazol-6-yl)-5-
methylpyrimidine-2,4-diamine;
N2-((4-aminosulfonyl)phenyl)-N4-(3-methyl-2-oxo-2,3-dihydrobenzo[d]oxazol-5-yl)-5-
methylpyrimidine-2,4-diamine;
N2-((4-aminosulfonyl)phenyl)-N4-(3-methyl-2-oxo-2,3-dihydrobenzo[d]oxazol-5-yl)-5-
fluoropyrimidine-2,4-diamine;
N2-((4-fluoro-3-methylsulfonylamino)phenyl)-N4-(2-oxo-2,3-dihydrobenzo[d]oxazol-5-
yl)-5-methylpyrimidine-2,4-diamine;
N2-((4-fluoro-3-methylsulfonylamino)phenyl)-N4-(2-oxo-2,3-dihydrobenzo[d]oxazol-5-
yl)-5-fluoropyrimidine-2,4-diamine;
N2-((4-fluoro-3-methylsulfonylamino)phenyl)-N4-(2-oxo-2,3-dihydrobenzo[d]oxazol-6-
yl)-5-methylpyrimidine-2,4-diamine;
N2-((4-fluoro-3-methylsulfonylamino)phenyl)-N4-(3-methyl-2-oxo-2,3-
dihydrobenzo[d]oxazol-5-yl)-5-methylpyrimidine-2,4-diamine;
N2-((4-fluoro-3-methylsulfonylamino)phenyl)-N4-(3-methyl-2-oxo-2,3-
dihydrobenzo[d]oxazol-6-yl)-5-methylpyrimidine-2,4-diamine;
N2-((4-fluoro-3-methylsulfonylamino)phenyl)-N4-(3-methyl-2-oxo-2,3-
dihydrobenzo[d]oxazol-5-yl)-5-fluoropyrimidine-2,4-diamine;
N2-((4-fluoro-3-methylsulfonylamino)phenyl)-N4-(3-methyl-2-oxo-2,3-
dihydrobenzo[d]oxazol-6-yl)-5-fluoropyrimidine-2,4-diamine;
N2-((4-methylsulfonyl)phenyl)-N4-(2-oxo-2,3-dihydrobenzo[d]oxazol-6-yl)-5-
methylpyrimidine-2,4-diamine;
N2-((4-methylsulfonyl)phenyl)-N4-(3-methyl-2-oxo-2,3-dihydrobenzo[d]oxazol-6-yl)-5-
methylpyrimidine-2,4-diamine;
N2-((4-methylsulfonyl)phenyl)-N4-(3-methyl-2-oxo-2,3-dihydrobenzo[d]oxazol-5-yl)-5-
methylpyrimidine-2,4-diamine;
N2-((4-methylsulfonyl)phenyl)-N4-(3-methyl-2-oxo-2,3-dihydrobenzo[d]oxazol-5-yl)-5-
fluoropyrimidine-2,4-diamine;
N2-((4-methylsulfonylamino)phenyl)-N4-(2-oxo-2,3-dihydrobenzo[d]oxazol-6-yl)-5-
methylpyrimidine-2,4-diamine;
N2-((4-methylsulfonylamino)phenyl)-N4-(3-methyl-2-oxo-2,3-dihydrobenzo[d]oxazol-6-
yl)-5-fluoropyrimidine-2,4-diamine;
N2-((4-methylsulfonylamino)phenyl)-N4-(3-methyl-2-oxo-2,3-dihydrobenzo[d]oxazol-5-
yl)-5-methylpyrimidine-2,4-diamine;
N2-((4-methylsulfonylamino)phenyl)-N4-(3-methyl-2-oxo-2,3-dihydrobenzo[d]oxazol-6-
yl)-5-methylpyrimidine-2,4-diamine;
N2-((4-methylsulfonylamino)phenyl)-N4-(3-methyl-2-oxo-2,3-dihydrobenzo[d]oxazol-5-
yl)-5-fluoropyrimidine-2,4-diamine;
N2-((4-methylsulfonyl-N-methylamino)phenyl)-N4-(2-oxo-2,3-dihydrobenzo[d]oxazol-5-
yl)-5-methylpyrimidine-2,4-diamine;
N2-((4-methylsulfonyl-N-methylamino)phenyl)-N4-(2-oxo-2,3-dihydrobenzo[d]oxazol-5-
yl)-5-fluoropyrimidine-2,4-diamine;
N2-((4-methylsulfonyl-N-methylamino)phenyl)-N4-(3-methyl-2-oxo-2,3-
dihydrobenzo[d]oxazol-5-yl)-5-methylpyrimidine-2,4-diamine;
N2-((4-methylsulfonyl-N-methylamino)phenyl)-N4-(3-methyl-2-oxo-2,3-
dihydrobenzo[d]oxazol-6-yl)-5-methylpyrimidine-2,4-diamine;
N2-((4-methylsulfonyl-N-methylamino)phenyl)-N4-(3-methyl-2-oxo-2,3-
dihydrobenzo[d]oxazol-6-yl)-5-fluoropyrimidine-2,4-diamine;
N2-(1,3-dihydro-isobenzofuran-5-yl)-5-methyl-N4-(2-oxo-2,3-dihydro-1,3-benzoxazol-5-
yl)-2,4-pyrimidinediamine;
N2-(1,3-dimethyl-1H-pyrazolo[3,4-b]pyridin-5-yl)-5-methyl-N4-(2-oxo-2,3-dihydro-1,3-
benzoxazol-5-yl)-2,4-pyrimidinediamine;
N2-(2,2-difluoro-2H-1,3-benzodioxol-5-yl)-5-methyl-N4-(2-oxo-2,3-dihydro-1,3-
benzoxazol-5-yl)-2,4-pyrimidinediamine;
N2-(2,2-dimethyl-2H-1,3-benzodioxol-5-yl)-5-methyl-N4-(2-oxo-2,3-dihydro-1,3-
benzoxazol-5-yl)-2,4-pyrimidinediamine;
N2-(2,3-dimethoxypyridin-5-yl)-5-methyl-N4-(2-oxo-2,3-dihydro-1,3-benzoxazol-5-yl)-
2,4-pyrimidinediamine;
N2-(2,3-dimethylpyridin-5-yl)-5-methyl-N4-(2-oxo-2,3-dihydro-1,3-benzoxazol-5-yl)-2,4-
pyrimidinediamine;
N2-(2,6-dimethylpyridin-4-yl)-5-methyl-N4-(2-oxo-2,3-dihydro-1,3-benzoxazol-5-yl)-2,4-
pyrimidinediamine;
N2-(2-isopropoxypyridin-5-yl)-5-methyl-N4-(2-oxo-2,3-dihydro-1,3-benzoxazol-5-yl)-2,4-
pyrimidinediamine;
N2-(2-methoxy-3-methylpyridin-5-yl)-5-methyl-N4-(2-oxo-2,3-dihydro-1,3-benzoxazol-5-
yl)-2,4-pyrimidinediamine;
N2-(2-methoxy-3-trifluoromethylpyridin-5-yl)-5-methyl-N4-(2-oxo-2,3-dihydro-1,3-
benzoxazol-5-yl)-2,4-pyrimidinediamine;
N2-(2-methoxypyridin-4-yl)-5-methyl-N4-(2-oxo-2,3-dihydro-1,3-benzoxazol-5-yl)-2,4-
pyrimidinediamine;
N2-(2-methoxypyridin-5-yl)-5-methyl-N4-(2-oxo-2,3-dihydro-1,3-benzoxazol-5-yl)-2,4-
pyrimidinediamine;
N2-(3-(difluoromethoxy)-4-methoxyphenyl)-N4-(benzo[d]oxazol-2(3H)-on-5-yl)-5-
methylpyrimidine-2,4-diamine;
N2-(3-(methylsulfonylamino)phenyl)-N4-(1H-benzo[d]imidazol-2(3H)-on-5-yl)-5-
methylpyrimidine-2,4-diamine;
N2-(3,4,5-trimethyl)phenyl-5-methyl-N4-(2-oxo-2,3-dihydro-1,3-benzoxazol-5-yl)-2,4-
pyrimidinediamine;
N2-(3,4-difluoro)phenyl-5-methyl-N4-(2-oxo-2,3-dihydro-1,3-benzoxazol-5-yl)-2,4-
pyrimidinediamine;
N2-(3,4-dimethoxy-5-trifluoromethyl)phenyl-5-methyl-N4-(2-oxo-2,3-dihydro-1,3-
benzoxazol-5-yl)-2,4-pyrimidinediamine;
N2-(3,4-dimethyl-2-fluoro)phenyl-5-methyl-N4-(2-oxo-2,3-dihydro-1,3-benzoxazol-5-yl)-
2,4-pyrimidinediamine;
N2-(3,4-dimethyl-5-fluoro)phenyl-5-methyl-N4-(2-oxo-2,3-dihydro-1,3-benzoxazol-5-yl)-
2,4-pyrimidinediamine;
N2-(3,4-dimethyl-5-hydroxymethyl)phenyl-5-methyl-N4-(2-oxo-2,3-dihydro-1,3-
benzoxazol-5-yl)-2,4-pyrimidinediamine;
N2-(3,4-dimethyl-5-methoxycarbonyl)phenyl-5-methyl-N4-(2-oxo-2,3-dihydro-1,3-
benzoxazol-5-yl)-2,4-pyrimidinediamine;
N2-(3,4-ethylenedioxy)phenyl-5-methyl-N4-(2-oxo-2,3-dihydro-1,3-benzoxazol-5-yl)-2,4-
pyrimidinediamine;
N2-(3,5-dichloro-4-difluoromethoxy)phenyl-5-methyl-N4-(2-oxo-2,3-dihydro-1,3-
benzoxazol-5-yl)-2,4-pyrimidinediamine;
N2-(3,5-difluoro)phenyl-5-methyl-N4-(2-oxo-2,3-dihydro-1,3-benzoxazol-5-yl)-2,4-
pyrimidinediamine;
N2-(3,5-difluoro-4-difluoromethoxy)phenyl-5-methyl-N4-(2-oxo-2,3-dihydro-1,3-
benzoxazol-5-yl)-2,4-pyrimidinediamine;
N2-(3,5-difluoro-4-methoxy)phenyl-5-methyl-N4-(2-oxo-2,3-dihydro-1,3-benzoxazol-5-
yl)-2,4-pyrimidinediamine;
N2-(3,5-difluoro-4-trifluoromethyl)phenyl-5-methyl-N4-(2-oxo-2,3-dihydro-1,3-
benzoxazol-5-yl)-2,4-pyrimidinediamine;
N2-(3,5-diisopropyl-4-methoxy)phenyl-5-methyl-N4-(2-oxo-2,3-dihydro-1,3-benzoxazol-5-
yl)-2,4-pyrimidinediamine;
N2-(3,5-dimethoxy)phenyl-5-methyl-N4-(2-oxo-2,3-dihydro-1,3-benzoxazol-5-yl)-2,4-
pyrimidinediamine;
N2-(3,5-dimethyl)phenyl-5-methyl-N4-(2-oxo-2,3-dihydro-1,3-benzoxazol-5-yl)-2,4-
pyrimidinediamine;
N2-(3,5-dimethyl-4-fluoro)phenyl-5-fluoro-N4-(2-oxo-2,3-dihydro-1,3-benzoxazol-5-yl)-
2,4-pyrimidinediamine;
N2-(3,5-dimethyl-4-fluoro)phenyl-5-methyl-N4-(2-oxo-2,3-dihydro-1,3-benzoxazol-5-yl)-
2,4-pyrimidinediamine benzenesulfonic acid salt;
N2-(3,5-dimethyl-4-fluoro)phenyl-5-methyl-N4-(2-oxo-2,3-dihydro-1,3-benzoxazol-5-yl)-
2,4-pyrimidinediamine sulfuric acid salt;
N2-(3,5-dimethyl-4-fluoro)phenyl-5-methyl-N4-(2-oxo-2,3-dihydro-1,3-benzoxazol-5-yl)-
2,4-pyrimidinediamine;
N2-(3,5-dimethyl-4-fluoro)phenyl-5-methyl-N4-(2-oxo-2,3-dihydro-1,3-benzoxazol-5-yl)-
2,4-pyrimidinediamine sodium salt;
N2-(3,5-dimethyl-4-fluoro)phenyl-5-methyl-N4-[3-(phosphonooxy)methyl-2-oxo-2,3-
dihydro-1,3-benzoxazol-5-yl]-2,4-pyrimidinediamine bis-sodium salt;
N2-(3,5-dimethyl-4-hydroxymethyl)phenyl-5-methyl-N4-(2-oxo-2,3-dihydro-1,3-
benzoxazol-5-yl)-2,4-pyrimidinediamine;
N2-(3,5-dimethyl-4-methoxy)phenyl-5-methyl-N4-(2-oxo-2,3-dihydro-1,3-benzoxazol-5-
yl)-2,4-pyrimidinediamine;
N2-(3,5-dimethyl-4-methoxycarbonyl)phenyl-5-methyl-N4-(2-oxo-2,3-dihydro-1,3-
benzoxazol-5-yl)-2,4-pyrimidinediamine;
N2-(3,5-dimethyl-4-methoxymethyl)phenyl-5-methyl-N4-(2-oxo-2,3-dihydro-1,3-
benzoxazol-5-yl)-2,4-pyrimidinediamine;
N2-(3,5-di-tert-butyl)phenyl-5-methyl-N4-(2-oxo-2,3-dihydro-1,3-benzoxazol-5-yl)-2,4-
pyrimidinediamine;
N2-(3-bromo)phenyl-5-methyl-N4-(2-oxo-2,3-dihydro-1,3-benzoxazol-5-yl)-2,4-
pyrimidinediamine;
N2-(3-carboxamide-5-trifluoromethyl)phenyl-5-methyl-N4-(2-oxo-2,3-dihydro-1,3-
benzoxazol-5-yl)-2,4-pyrimidinediamine;
N2-(3-carboxy-4,5-dimethyl)phenyl-5-methyl-N4-(2-oxo-2,3-dihydro-1,3-benzoxazol-5-
yl)-2,4-pyrimidinediamine;
N2-(3-carboxy-4-fluoro-5-methyl)phenyl-5-methyl-N4-(2-oxo-2,3-dihydro-1,3-benzoxazol-
5-yl)-2,4-pyrimidinediamine;
N2-(3-carboxy-4-methyl)phenyl-5-methyl-N4-(2-oxo-2,3-dihydro-1,3-benzoxazol-5-yl)-
2,4-pyrimidinediamine;
N2-(3-chloro-4-difluoromethoxy)phenyl-5-methyl-N4-(2-oxo-2,3-dihydro-1,3-benzoxazol-
5-yl)-2,4-pyrimidinediamine;
N2-(3-chloro-4-fluoro)phenyl-5-methyl-N4-(2-oxo-2,3-dihydro-1,3-benzoxazol-5-yl)-2,4-
pyrimidinediamine;
N2-(3-chloro-4-methyl)phenyl-5-methyl-N4-(2-oxo-2,3-dihydro-1,3-benzoxazol-5-yl)-2,4-
pyrimidinediamine;
N2-(3-chloro-4-trifluoromethoxy)phenyl-5-methyl-N4-(2-oxo-2,3-dihydro-1,3-benzoxazol-
5-yl)-2,4-pyrimidinediamine;
N2-(3-chloro-5-difluoromethoxy)phenyl-5-methyl-N4-(2-oxo-2,3-dihydro-1,3-benzoxazol-
5-yl)-2,4-pyrimidinediamine;
N2-(3-chloro-5-trifluoromethoxy)phenyl-5-methyl-N4-(2-oxo-2,3-dihydro-1,3-benzoxazol-
5-yl)-2,4-pyrimidinediamine;
N2-(3-cyano-4-fluoro)phenyl-5-methyl-N4-(2-oxo-2,3-dihydro-1,3-benzoxazol-5-yl)-2,4-
pyrimidinediamine;
N2-(3-cyano-4-methoxy)phenyl-5-methyl-N4-(2-oxo-2,3-dihydro-1,3-benzoxazol-5-yl)-
2,4-pyrimidinediamine;
N2-(3-cyano-4-morpholino)phenyl-5-methyl-N4-(2-oxo-2,3-dihydro-1,3-benzoxazol-5-yl)-
2,4-pyrimidinediamine;
N2-(3-cyano-4-thiomorpholino)phenyl-5-methyl-N4-(2-oxo-2,3-dihydro-1,3-benzoxazol-5-
yl)-2,4-pyrimidinediamine;
N2-(3-cyano-5-difluoromethoxy)phenyl-5-methyl-N4-(2-oxo-2,3-dihydro-1,3-benzoxazol-
5-yl)-2,4-pyrimidinediamine;
N2-(3-difluoromethoxy)phenyl-5-methyl-N4-(2-oxo-2,3-dihydro-1,3-benzoxazol-5-yl)-2,4-
pyrimidinediamine;
N2-(3-difluoromethoxy-4-fluoro)phenyl-5-methyl-N4-(2-oxo-2,3-dihydro-1,3-benzoxazol-
5-yl)-2,4-pyrimidinediamine;
N2-(3-difluoromethoxy-4-methyl)phenyl-5-methyl-N4-(2-oxo-2,3-dihydro-1,3-benzoxazol-
5-yl)-2,4-pyrimidinediamine;
N2-(3-difluoromethoxy-5-fluoro)phenyl-5-methyl-N4-(2-oxo-2,3-dihydro-1,3-benzoxazol-
5-yl)-2,4-pyrimidinediamine;
N2-(3-difluoromethoxy-5-methyl)phenyl-5-methyl-N4-(2-oxo-2,3-dihydro-1,3-benzoxazol-
5-yl)-2,4-pyrimidinediamine;
N2-(3-difluoromethoxy-5-trifluoromethyl)phenyl-5-methyl-N4-(2-oxo-2,3-dihydro-1,3-
benzoxazol-5-yl)-2,4-pyrimidinediamine;
N2-(3-dimethylaminocarbonylmethoxy)phenyl-5-methyl-N4-(2-oxo-2,3-dihydro-1,3-
benzoxazol-5-yl)-2,4-pyrimidinediamine;
N2-(3-fluoro)phenyl-5-methyl-N4-(2-oxo-2,3-dihydro-1,3-benzoxazol-5-yl)-2,4-
pyrimidinediamine;
N2-(3-fluoro-4-methoxy)phenyl-5-methyl-N4-(2-oxo-2,3-dihydro-1,3-benzoxazol-5-yl)-
2,4-pyrimidinediamine;
N2-(3-fluoro-4-methyl)phenyl-5-methyl-N4-(2-oxo-2,3-dihydro-1,3-benzoxazol-5-yl)-2,4-
pyrimidinediamine;
N2-(3-fluoro-4-trifluoromethoxy)phenyl-5-methyl-N4-(2-oxo-2,3-dihydro-1,3-benzoxazol-
5-yl)-2,4-pyrimidinediamine;
N2-(3-fluoro-5-methoxy)phenyl-5-methyl-N4-(2-oxo-2,3-dihydro-1,3-benzoxazol-5-yl)-
2,4-pyrimidinediamine;
N2-(3-fluoro-5-trifluoromethyl)phenyl-5-methyl-N4-(2-oxo-2,3-dihydro-1,3-benzoxazol-5-
yl)-2,4-pyrimidinediamine;
N2-(3-fluoropyridin-4-yl)-5-methyl-N4-(2-oxo-2,3-dihydro-1,3-benzoxazol-5-yl)-2,4-
pyrimidinediamine;
N2-(3-fluoropyridin-4-yl)-5-methyl-N4-(2-oxo-2,3-dihydro-1,3-benzoxazol-5-yl)-2,4-
pyrimidinediamine trifluoroacetic acid salt;
N2-(3-isopropoxy-5-trifluoromethyl)phenyl-5-methyl-N4-(2-oxo-2,3-dihydro-1,3-
benzoxazol-5-yl)-2,4-pyrimidinediamine;
N2-(3-methoxy)phenyl-5-methyl-N4-(2-oxo-2,3-dihydro-1,3-benzoxazol-5-yl)-2,4-
pyrimidinediamine;
N2-(3-methoxy-2-methyl)phenyl-5-methyl-N4-(2-oxo-2,3-dihydro-1,3-benzoxazol-5-yl)-
2,4-pyrimidinediamine;
N2-(3-methoxy-4-methoxycarbonyl)phenyl-5-methyl-N4-(2-oxo-2,3-dihydro-1,3-
benzoxazol-5-yl)-2,4-pyrimidinediamine;
N2-(3-methoxy-4-methyl)phenyl-5-methyl-N4-(2-oxo-2,3-dihydro-1,3-benzoxazol-5-yl)-
2,4-pyrimidinediamine;
N2-(3-methoxy-4-trifluoromethyl)phenyl-5-methyl-N4-(2-oxo-2,3-dihydro-1,3-benzoxazol-
5-yl)-2,4-pyrimidinediamine;
N2-(3-methoxy-5-trifluoromethyl)phenyl-5-methyl-N4-(2-oxo-2,3-dihydro-1,3-benzoxazol-
5-yl)-2,4-pyrimidinediamine;
N2-(3-methoxy-5-trifluoromethyl)phenyl-5-methyl-N4-(2-oxo-2,3-dihydro-1,3-benzoxazol-
5-yl)-2,4-pyrimidinediamine besylate salt;
N2-(3-methoxy-5-trifluoromethyl)phenyl-5-methyl-N4-(2-oxo-2,3-dihydro-1,3-benzoxazol-
5-yl)-2,4-pyrimidinediamine tosylate salt;
N2-(3-methoxy-5-trifluoromethyl)phenyl-5-methyl-N4-(2-oxo-2,3-dihydro-1,3-benzoxazol-
5-yl)-2,4-pyrimidinediamine mesylate salt;
N2-(3-methoxy-5-trifluoromethyl)phenyl-5-methyl-N4-(2-oxo-2,3-dihydro-1,3-benzoxazol-
5-yl)-2,4-pyrimidinediamine sulfate salt;
N2-(3-methoxy-5-trifluoromethyl)phenyl-5-methyl-N4-(2-oxo-2,3-dihydro-1,3-benzoxazol-
5-yl)-2,4-pyrimidinediamine hydrogen chloride salt;
N2-(3-methoxy-5-trifluoromethyl)phenyl-5-methyl-N4-(2-oxo-2,3-dihydro-1,3-benzoxazol-
5-yl)-2,4-pyrimidinediamine sodium salt;
N2-(3-methoxy-5-trifluoromethyl)phenyl-5-methyl-N4-(2-oxo-2,3-dihydro-1,3-benzoxazol-
5-yl)-2,4-pyrimidinediamine choline salt;
N2-(3-methoxy-5-trifluoromethyl)phenyl-5-methyl-N4-[3-(phosphonooxy)methyl-2-oxo-
2,3-dihydro-1,3-benzoxazol-5-yl]-2,4-pyrimidinediamine;
N2-(3-methoxy-5-trifluoromethyl)phenyl-5-methyl-N4-[3-(phosphonooxy)methyl-2-oxo-
2,3-dihydro-1,3-benzoxazol-5-yl]-2,4-pyrimidinediamine bis-sodium salt;
N2-(3-methoxycarbonylmethoxy)phenyl-5-methyl-N4-(2-oxo-2,3-dihydro-1,3-benzoxazol-
5-yl)-2,4-pyrimidinediamine;
N2-(4-(methylsulfonylamino)phenyl)-N4-(1H-benzo[d]imidazol-2(3H)-on-5-yl)-5-
methylpyrimidine-2,4-diamine;
N2-(4-(methylsulfonylamino)phenyl)-N4-(1H-benzo[d]imidazol-2(3H)-on-5-yl)-5-
fluoropyrimidine-2,4-diamine;
N2-(4,5-dimethylpyridin-2-yl)-5-methyl-N4-(2-oxo-2,3-dihydro-1,3-benzoxazol-5-yl)-2,4-
pyrimidinediamine;
N2-(4,6-dimethylpyridin-2-yl)-5-methyl-N4-(2-oxo-2,3-dihydro-1,3-benzoxazol-5-yl)-2,4-
pyrimidinediamine;
N2-(4-aminocarbonylmethoxy)phenyl-5-methyl-N4-(2-oxo-2,3-dihydro-1,3-benzoxazol-5-
yl)-2,4-pyrimidinediamine;
N2-(4-aminosulfonyl)phenyl)-N4-(3-methyl-2-oxo-2,3-dihydrobenzo[d]oxazol-6-yl)-5-
fluoropyrimidine-2,4-diamine;
N2-(4-benzyloxycarbonyl-3,5-dimethyl)phenyl-5-methyl-N4-(2-oxo-2,3-dihydro-1,3-
benzoxazol-5-yl)-2,4-pyrimidinediamine;
N2-(4-bromo)phenyl-5-methyl-N4-(2-oxo-2,3-dihydro-1,3-benzoxazol-5-yl)-2,4-
pyrimidinediamine;
N2-(4-bromo-3,5-dimethyl)phenyl-5-methyl-N4-(2-oxo-2,3-dihydro-1,3-benzoxazol-5-yl)-
2,4-pyrimidinediamine;
N2-(4-carboxy-3,5-dimethyl)phenyl-5-methyl-N4-(2-oxo-2,3-dihydro-1,3-benzoxazol-5-
yl)-2,4-pyrimidinediamine;
N2-(4-chloro-3,5-dimethyl)phenyl-5-methyl-N4-(2-oxo-2,3-dihydro-1,3-benzoxazol-5-yl)-
2,4-pyrimidinediamine;
N2-(4-chloro-3-cyano-5-ethyl)phenyl-5-methyl-N4-(2-oxo-2,3-dihydro-1,3-benzoxazol-5-
yl)-2,4-pyrimidinediamine;
N2-(4-chloro-3-difluoromethoxy)phenyl-5-methyl-N4-(2-oxo-2,3-dihydro-1,3-benzoxazol-
5-yl)-2,4-pyrimidinediamine;
N2-(4-chloro-3-fluoro)phenyl-5-methyl-N4-(2-oxo-2,3-dihydro-1,3-benzoxazol-5-yl)-2,4-
pyrimidinediamine;
N2-(4-chloro-3-trifluoromethoxy)phenyl-5-methyl-N4-(2-oxo-2,3-dihydro-1,3-benzoxazol-
5-yl)-2,4-pyrimidinediamine;
N2-(4-cyano-3-difluoromethoxy)phenyl-5-methyl-N4-(2-oxo-2,3-dihydro-1,3-benzoxazol-
5-yl)-2,4-pyrimidinediamine;
N2-(4-cyano-3-methoxy)phenyl-5-methyl-N4-(2-oxo-2,3-dihydro-1,3-benzoxazol-5-yl)-
2,4-pyrimidinediamine;
N2-(4-cyano-3-trifluoromethyl)phenyl-5-methyl-N4-(2-oxo-2,3-dihydro-1,3-benzoxazol-5-
yl)-2,4-pyrimidinediamine;
N2-(4-difluoromethoxy)phenyl-5-methyl-N4-(2-oxo-2,3-dihydro-1,3-benzoxazol-5-yl)-2,4-
pyrimidinediamine;
N2-(4-difluoromethoxy-3,5-dimethyl)phenyl-5-methyl-N4-(2-oxo-2,3-dihydro-1,3-
benzoxazol-5-yl)-2,4-pyrimidinediamine;
N2-(4-difluoromethoxy-3-ethoxy)phenyl-5-methyl-N4-(2-oxo-2,3-dihydro-1,3-benzoxazol-
5-yl)-2,4-pyrimidinediamine;
N2-(4-difluoromethoxy-3-fluoro)phenyl-5-methyl-N4-(2-oxo-2,3-dihydro-1,3-benzoxazol-
5-yl)-2,4-pyrimidinediamine;
N2-(4-difluoromethoxy-3-methyl)phenyl-5-methyl-N4-(2-oxo-2,3-dihydro-1,3-benzoxazol-
5-yl)-2,4-pyrimidinediamine;
N2-(4-fluoro-3-hydroxymethyl-5-methyl)phenyl-5-methyl-N4-(2-oxo-2,3-dihydro-1,3-
benzoxazol-5-yl)-2,4-pyrimidinediamine;
N2-(4-fluoro-3-methoxy)phenyl-5-methyl-N4-(2-oxo-2,3-dihydro-1,3-benzoxazol-5-yl)-
2,4-pyrimidinediamine;
N2-(4-fluoro-3-methoxycarbonyl-5-methyl)phenyl-5-methyl-N4-(2-oxo-2,3-dihydro-1,3-
benzoxazol-5-yl)-2,4-pyrimidinediamine;
N2-(4-fluoro-3-methyl)phenyl-5-methyl-N4-(2-oxo-2,3-dihydro-1,3-benzoxazol-5-yl)-2,4-
pyrimidinediamine;
N2-(4-fluoro-3-trifluoromethoxy)phenyl-5-methyl-N4-(2-oxo-2,3-dihydro-1,3-benzoxazol-
5-yl)-2,4-pyrimidinediamine;
N2-(4-fluoro-3-trifluoromethyl)phenyl-5-methyl-N4-(2-oxo-2,3-dihydro-1,3-benzoxazol-5-
yl)-2,4-pyrimidinediamine;
N2-(4-methoxy-3-trifluoromethyl)phenyl-5-methyl-N4-(2-oxo-2,3-dihydro-1,3-benzoxazol-
5-yl)-2,4-pyrimidinediamine;
N2-(4-methoxypyridin-2-yl)-5-methyl-N4-(2-oxo-2,3-dihydro-1,3-benzoxazol-5-yl)-2,4-
pyrimidinediamine;
N2-(4-methylsulfonyl)phenyl)-N4-(3-methyl-2-oxo-2,3-dihydrobenzo[d]oxazol-6-yl)-5-
fluoropyrimidine-2,4-diamine;
N2-(4-n-butyl-3-methyl)phenyl-5-methyl-N4-(2-oxo-2,3-dihydro-1,3-benzoxazol-5-yl)-2,4-
pyrimidinediamine;
N2-(4-tert-butoxycarbonyl-3,5-dimethyl)phenyl-5-methyl-N4-(2-oxo-2,3-dihydro-1,3-
benzoxazol-5-yl)-2,4-pyrimidinediamine;
N2-(4-trifluoromethylthio)phenyl-5-methyl-N4-(2-oxo-2,3-dihydro-1,3-benzoxazol-5-yl)-
2,4-pyrimidinediamine;
N2-(5,6-dimethylpyridin-2-yl)-5-methyl-N4-(2-oxo-2,3-dihydro-1,3-benzoxazol-5-yl)-2,4-
pyrimidinediamine;
N2-(5-cyano-4-methylpyridin-2-yl)-5-methyl-N4-(2-oxo-2,3-dihydro-1,3-benzoxazol-5-yl)-
2,4-pyrimidinediamine;
N2-(5-cyano-6-methylpyridin-2-yl)-5-methyl-N4-(2-oxo-2,3-dihydro-1,3-benzoxazol-5-yl)-
2,4-pyrimidinediamine;
N2-(5-methoxypyridin-3-yl)-5-methyl-N4-(2-oxo-2,3-dihydro-1,3-benzoxazol-5-yl)-2,4-
pyrimidinediamine;
N2-(6-(4-methylpiperazin-1-yl)pyridin-3-yl)-N4-(3-isopropylbenzo[d]oxazol-2(3H)-on-5-
yl)-5-methylpyrimidine-2,4-diamine;
N2-(6-(4-methylpiperazin-1-yl)pyridin-3-yl)-N4-(3-methyl-2-oxo-2,3-
dihydrobenzo[d]oxazol-5-yl)-5-methylpyrimidine-2,4-diamine;
N2-(6-(4-methylpiperazin-1-yl)pyridin-3-yl)-N4-(3-methyl-2-oxo-2,3-
dihydrobenzo[d]oxazol-6-yl)-5-methylpyrimidine-2,4-diamine trifluoroacetate salt;
N2-(6-(4-methylpiperazin-1-yl)pyridin-3-yl)-N4-(3-methyl-2-oxo-2,3-
dihydrobenzo[d]oxazol-5-yl)-5-fluoropyrimidine-2,4-diamine;
N2-(6-methoxypyridin-2-yl)-5-methyl-N4-(2-oxo-2,3-dihydro-1,3-benzoxazol-5-yl)-2,4-
pyrimidinediamine;
N2-[2-(1,4-diazabicylco[3.2.2]nonan-4-yl)-3-fluoropyridin-5-yl]-5-methyl-N4-(2-oxo-2,3-
dihydro-1,3-benzoxazol-5-yl)-2,4-pyrimidinediamine;
N2-[2-(1,4-diazabicylco[4.4.0]decan-4-yl)pyridin-5-yl]-5-methyl-N4-(2-oxo-2,3-dihydro-
1,3-benzoxazol-5-yl)-2,4-pyrimidinediamine;
N2-[2-(1-aminocarbonyl-1-methyl)ethoxypyridin-5-yl]-5-methyl-N4-(2-oxo-2,3-dihydro-
1,3-benzoxazol-5-yl)-2,4-pyrimidinediamine;
N2-[2-(2-hydroxy)ethoxypyridin-5-yl]-5-methyl-N4-(2-oxo-2,3-dihydro-1,3-benzoxazol-5-
yl)-2,4-pyrimidinediamine;
N2-[2-(2-methoxy)ethoxypyridin-5-yl]-5-methyl-N4-(2-oxo-2,3-dihydro-1,3-benzoxazol-5-
yl)-2,4-pyrimidinediamine;
N2-[2-(3-ethyl-3,7-diazabicyclo[3.3.0]octan-7-yl)-3-trifluoromethylpyridin-5-yl]-5-methyl-
N4-(2-oxo-2,3-dihydro-1,3-benzoxazol-5-yl)-2,4-pyrimidinediamine;
N2-[2-(3-hydroxy)propoxypyridin-5-yl]-5-methyl-N4-(2-oxo-2,3-dihydro-1,3-benzoxazol-
5-yl)-2,4-pyrimidinediamine;
N2-[2-(3-methoxy)propoxypyridin-5-yl]-5-methyl-N4-(2-oxo-2,3-dihydro-1,3-benzoxazol-
5-yl)-2,4-pyrimidinediamine;
N2-[2-(3-N,N-dimethylamino-8-azabicyclo[3.2.1]octan-8-yl)pyridin-5-yl]-5-methyl-N4-(2-
oxo-2,3-dihydro-1,3-benzoxazol-5-yl)-2,4-pyrimidinediamine;
N2-[2-(4-isopropylpiperazino)pyridin-5-yl]-5-methyl-N4-(2-oxo-2,3-dihydro-1,3-
benzoxazol-5-yl)-2,4-pyrimidinediamine;
N2-[2-(4R-hydroxy-2-methylidene-pyrrolidin-1-yl)pyridin-5-yl]-5-methyl-N4-(2-oxo-2,3-
dihydro-1,3-benzoxazol-5-yl)-2,4-pyrimidinediamine;
N2-[2-(cis-3,5-dimethylpiperazino)pyridin-5-yl]-5-methyl-N4-(2-oxo-2,3-dihydro-1,3-
benzoxazol-5-yl)-2,4-pyrimidinediamine;
N2-[2-(R-1,4-diazabicylco[4.3.0]nonan-4-yl)pyridin-5-yl]-5-methyl-N4-(2-oxo-2,3-
dihydro-1,3-benzoxazol-5-yl)-2,4-pyrimidinediamine;
N2-[2-(S-1,4-diazabicylco[4.3.0]nonan-4-yl)-3-trifluoromethylpyridin-5-yl]-5-methyl-N4-
(2-oxo-2,3-dihydro-1,3-benzoxazol-5-yl)-2,4-pyrimidinediamine;
N2-[2-(trans-2,5-dimethylpiperazino)pyridin-5-yl]-5-methyl-N4-(2-oxo-2,3-dihydro-1,3-
benzoxazol-5-yl)-2,4-pyrimidinediamine;
N2-[2,2-dimethyl-2H-1,4-benzoxazin-3(4H)-one-7-yl]-5-methyl-N4-(2-oxo-2,3-dihydro-
1,3-benzoxazol-5-yl)-2,4-pyrimidinediamine;
N2-[2,2-dimethyl-4-(2-methoxyethyl)-2H-pyrido[3,2-b][1,4]oxazin-3(4H)-one-7-yl]-5-
methyl-N4-(2-oxo-2,3-dihydro-1,3-benzoxazol-5-yl)-2,4-pyrimidinediamine;
N2-[3-(1-aminocarbonyl-1-methyl)ethoxy]phenyl-5-methyl-N4-(2-oxo-2,3-dihydro-1,3-
benzoxazol-5-yl)-2,4-pyrimidinediamine;
N2-[3-(1-aminocarbonyl-1-methyl)ethoxy-4-fluoro]phenyl-5-methyl-N4-(2-oxo-2,3-
dihydro-1,3-benzoxazol-5-yl)-2,4-pyrimidinediamine;
N2-[3-(1-aminocarbonyl-1-methyl)ethoxy-4-methyl]phenyl-5-methyl-N4-(2-oxo-2,3-
dihydro-1,3-benzoxazol-5-yl)-2,4-pyrimidinediamine;
N2-[3-(1-aminocarbonyl-1-methyl)ethoxy-5-fluoro]phenyl-5-methyl-N4-(2-oxo-2,3-
dihydro-1,3-benzoxazol-5-yl)-2,4-pyrimidinediamine;
N2-[3-(1-cyano-1-methyl)ethoxy]phenyl-5-methyl-N4-(2-oxo-2,3-dihydro-1,3-benzoxazol-
5-yl)-2,4-pyrimidinediamine;
N2-[3-(1-cyano-1-methyl)ethoxy-4-fluoro]phenyl-5-methyl-N4-(2-oxo-2,3-dihydro-1,3-
benzoxazol-5-yl)-2,4-pyrimidinediamine;
N2-[3-(1-cyano-1-methyl)ethoxy-4-methyl]phenyl-5-methyl-N4-(2-oxo-2,3-dihydro-1,3-
benzoxazol-5-yl)-2,4-pyrimidinediamine;
N2-[3-(1-cyano-1-methyl)ethoxy-5-fluoro]phenyl-5-methyl-N4-(2-oxo-2,3-dihydro-1,3-
benzoxazol-5-yl)-2,4-pyrimidinediamine;
N2-[3-(1-hydroxy-2,2,2-trifluoroethyl)]phenyl-5-methyl-N4-(2-oxo-2,3-dihydro-1,3-
benzoxazol-5-yl)-2,4-pyrimidinediamine;
N2-[3-(1-methoxy-2,2,2-trifluoroethyl)]phenyl-5-methyl-N4-(2-oxo-2,3-dihydro-1,3-
benzoxazol-5-yl)-2,4-pyrimidinediamine;
N2-[3-(2-methoxy)ethoxy-5-trifluoromethyl]phenyl-5-methyl-N4-(2-oxo-2,3-dihydro-1,3-
benzoxazol-5-yl)-2,4-pyrimidinediamine;
N2-[3-(4-acetyl)piperazino]phenyl-5-methyl-N4-(2-oxo-2,3-dihydro-1,3-benzoxazol-5-yl)-
2,4-pyrimidinediamine;
N2-[3-(4-ethoxycarbonyl)piperazino]phenyl-5-methyl-N4-(2-oxo-2,3-dihydro-1,3-
benzoxazol-5-yl)-2,4-pyrimidinediamine;
N2-[3-(aminocarbonylmethoxy)]phenyl-5-methyl-N4-(2-oxo-2,3-dihydro-1,3-benzoxazol-
5-yl)-2,4-pyrimidinediamine;
N2-[3-(cyclopropylaminocarbonylmethoxy)]phenyl-5-methyl-N4-(2-oxo-2,3-dihydro-1,3-
benzoxazol-5-yl)-2,4-pyrimidinediamine;
N2-[3-(cyclopropylaminocarbonylmethoxy)-4-methoxy]phenyl-5-fluoro-N4-(2-oxo-2,3-
dihydro-1,3-benzoxazol-5-yl)-2,4-pyrimidinediamine;
N2-[3-(cyclopropylaminocarbonylmethoxy)-4-methoxy]phenyl-5-methyl-N4-(2-oxo-2,3-
dihydro-1,3-benzoxazol-5-yl)-2,4-pyrimidinediamine;
N2-[3,5-bis(trifluoromethyl)]phenyl-5-methyl-N4-(2-oxo-2,3-dihydro-1,3-benzoxazol-5-
yl)-2,4-pyrimidinediamine;
N2-[3,5-difluoro-4-(4-methylpiperazino)]phenyl-5-methyl-N4-(2-oxo-2,3-dihydro-1,3-
benzoxazol-5-yl)-2,4-pyrimidinediamine;
N2-[3-aminocarbonyl-4-(4-methylpiperazino)]phenyl-5-methyl-N4-(2-oxo-2,3-dihydro-1,3-
benzoxazol-5-yl)-2,4-pyrimidinediamine;
N2-[3-chloro-2-(3-ethyl-3,7-diazabicyclo[3.3.0]octan-7-yl)pyridin-5-yl]-5-methyl-N4-(2-
oxo-2,3-dihydro-1,3-benzoxazol-5-yl)-2,4-pyrimidinediamine;
N2-[3-chloro-2-(4-methylpiperazino)pyridin-5-yl]-5-methyl-N4-(2-oxo-2,3-dihydro-1,3-
benzoxazol-5-yl)-2,4-pyrimidinediamine;
N2-[3-chloro-2-(octahydroisoindol-1-yl)pyridin-5-yl]-5-methyl-N4-(2-oxo-2,3-dihydro-1,3-
benzoxazol-5-yl)-2,4-pyrimidinediamine;
N2-[3-chloro-4-(4,4-difluoropiperidinyl)]phenyl-5-methyl-N4-(2-oxo-2,3-dihydro-1,3-
benzoxazol-5-yl)-2,4-pyrimidinediamine;
N2-[3-chloro-4-(4-ethylpiperazino)]phenyl-5-methyl-N4-(2-oxo-2,3-dihydro-1,3-
benzoxazol-5-yl)-2,4-pyrimidinediamine;
N2-[3-chloro-4-(pyridin-4-yl)]phenyl 5-methyl-N4-(2-oxo-2,3-dihydro-1,3-benzoxazol-5-
yl)-2,4-pyrimidinediamine;
N2-[3-cyano-4-(1H-pyrrol-1-yl)]phenyl-5-methyl-N4-(2-oxo-2,3-dihydro-1,3-benzoxazol-
5-yl)-2,4-pyrimidinediamine;
N2-[3-cyano-4-(4-methylpiperazino)]phenyl-5-methyl-N4-(2-oxo-2,3-dihydro-1,3-
benzoxazol-5-yl)-2,4-pyrimidinediamine;
N2-[3-cyano-4-(pyrrolidin-1-yl)]phenyl-5-methyl-N4-(2-oxo-2,3-dihydro-1,3-benzoxazol-
5-yl)-2,4-pyrimidinediamine;
N2-[3-fluoro-4-(4-methylpiperazino)]phenyl-5-methyl-N4-(2-oxo-2,3-dihydro-1,3-
benzoxazol-5-yl)-2,4-pyrimidinediamine;
N2-[3-hydroxymethyl-4-(4-methylpiperazino)]phenyl-5-methyl-N4-(2-oxo-2,3-dihydro-
1,3-benzoxazol-5-yl)-2,4-pyrimidinediamine;
N2-[3-methoxy-4-(4-methylpiperazino)]phenyl-5-methyl-N4-(2-oxo-2,3-dihydro-1,3-
benzoxazol-5-yl)-2,4-pyrimidinediamine;
N2-[4-(1-aminocarbonyl-1-methyl)ethoxy]phenyl-5-methyl-N4-(2-oxo-2,3-dihydro-1,3-
benzoxazol-5-yl)-2,4-pyrimidinediamine;
N2-[4-(1-aminocarbonyl-1-methyl)ethoxy-3,5-difluoro]phenyl-5-methyl-N4-(2-oxo-2,3-
dihydro-1,3-benzoxazol-5-yl)-2,4-pyrimidinediamine;
N2-[4-(1-aminocarbonyl-1-methyl)ethoxy-3,5-dimethyl]phenyl-5-methyl-N4-(2-oxo-2,3-
dihydro-1,3-benzoxazol-5-yl)-2,4-pyrimidinediamine;
N2-[4-(1-aminocarbonyl-1-methyl)ethoxy-3-methyl]phenyl-5-methyl-N4-(2-oxo-2,3-
dihydro-1,3-benzoxazol-5-yl)-2,4-pyrimidinediamine;
N2-[4-(1-cyano-1-methyl)ethoxy]phenyl-5-methyl-N4-(2-oxo-2,3-dihydro-1,3-benzoxazol-
5-yl)-2,4-pyrimidinediamine;
N2-[4-(2-methoxyethyl)-2H-1,4-benzoxazin-3(4H)-one-7-yl]-5-methyl-N4-(2-oxo-2,3-
dihydro-1,3-benzoxazol-5-yl)-2,4-pyrimidinediamine;
N2-[4-(4,4-difluoropiperidinyl)]phenyl-5-methyl-N4-(2-oxo-2,3-dihydro-1,3-benzoxazol-5-
yl)-2,4-pyrimidinediamine;
N2-[4-(4,4-difluoropiperidinyl)-3-fluoro]phenyl-5-methyl-N4-(2-oxo-2,3-dihydro-1,3-
benzoxazol-5-yl)-2,4-pyrimidinediamine;
N2-[4-(4,4-difluoropiperidinyl)-3-trifluoromethyl]phenyl-5-methyl-N4-(2-oxo-2,3-dihydro-
1,3-benzoxazol-5-yl)-2,4-pyrimidinediamine;
N2-[4-(4-acetyl)piperazino]phenyl-5-methyl-N4-(2-oxo-2,3-dihydro-1,3-benzoxazol-5-yl)-
2,4-pyrimidinediamine;
N2-[4-(4-ethoxycarbonyl)piperazino]phenyl-5-methyl-N4-(2-oxo-2,3-dihydro-1,3-
benzoxazol-5-yl)-2,4-pyrimidinediamine;
N2-[4-(4-ethylaminocarbonyl)piperazino]phenyl-5-methyl-N4-(2-oxo-2,3-dihydro-1,3-
benzoxazol-5-yl)-2,4-pyrimidinediamine;
N2-[4-(ethylaminocarbonylamino)]phenyl-5-methyl-N4-(2-oxo-2,3-dihydro-1,3-
benzoxazol-5-yl)-2,4-pyrimidinediamine;
N2-[4-(isopropoxycarbonylmethoxy)]phenyl-5-methyl-N4-(2-oxo-2,3-dihydro-1,3-
benzoxazol-5-yl)-2,4-pyrimidinediamine;
N2-[4-(N-acetyl-N-methyl)amino]phenyl-5-methyl-N4-(2-oxo-2,3-dihydro-1,3-benzoxazol-
5-yl)-2,4-pyrimidinediamine;
N2-[4-chloro-3-(3,4,5-trimethylpiperazino)]phenyl-5-methyl-N4-(2-oxo-2,3-dihydro-1,3-
benzoxazol-5-yl)-2,4-pyrimidinediamine;
N2-[4-chloro-3-(4-ethylpiperazino)]phenyl-5-methyl-N4-(2-oxo-2,3-dihydro-1,3-
benzoxazol-5-yl)-2,4-pyrimidinediamine;
N2-[4-methoxy-3-(2-methoxyethoxy)]phenyl-5-methyl-N4-(2-oxo-2,3-dihydro-1,3-
benzoxazol-5-yl)-2,4-pyrimidinediamine;
N2-[4-methyl-2-(4-methylpiperazino)pyridin-5-yl]-5-methyl-N4-(2-oxo-2,3-dihydro-1,3-
benzoxazol-5-yl)-2,4-pyrimidinediamine;
N2-[spiro(2,1′-cyclohexan)-1,3-benzodioxol-5-yl]-5-methyl-N4-(2-oxo-2,3-dihydro-1,3-
benzoxazol-5-yl)-2,4-pyrimidinediamine;
N2-{4-methoxy-3-[(pyridin-3-yl)methoxy]}phenyl-5-methyl-N4-(2-oxo-2,3-dihydro-1,3-
benzoxazol-5-yl)-2,4-pyrimidinediamine;
N2-{4-methoxy-3-[(pyridin-4-yl)methoxy]}phenyl-5-methyl-N4-(2-oxo-2,3-dihydro-1,3-
benzoxazol-5-yl)-2,4-pyrimidinediamine;
N2-{4-methoxy-3-[2-(dimethylamino)ethoxy]}phenyl-5-methyl-N4-(2-oxo-2,3-dihydro-
1,3-benzoxazol-5-yl)-2,4-pyrimidinediamine;
N2-ethyl-N2-(3,4,5-trimethyl)phenyl-5-methyl-N4-(2-oxo-2,3-dihydro-1,3-benzoxazol-5-
yl)-2,4-pyrimidinediamine;
N4-(1,3-dimethylbenzimidazolin-2-on-5-yl)-N2-((3-aminosulfonyl)phenyl)-5-
methylpyrimidine-2,4-diamine;
N4-(1,3-dimethylbenzimidazolin-2-on-5-yl)-N2-((3-aminosulfonyl)phenyl)-5-
fluoropyrimidine-2,4-diamine;
N4-(1,3-dimethylbenzimidazolin-2-on-5-yl)-N2-((3-methylsulfonyl)phenyl)-5-
methylpyrimidine-2,4-diamine;
N4-(1,3-dimethylbenzimidazolin-2-on-5-yl)-N2-((3-methylsulfonyl)phenyl)-5-
fluoropyrimidine-2,4-diamine;
N4-(1,3-dimethylbenzimidazolin-2-on-5-yl)-N2-((3-methylsulfonylamino)phenyl)-5-
methylpyrimidine-2,4-diamine;
N4-(1,3-dimethylbenzimidazolin-2-on-5-yl)-N2-((3-methylsulfonylamino)phenyl)-5-
fluoropyrimidine-2,4-diamine;
N4-(1,3-dimethylbenzimidazolin-2-on-5-yl)-N2-((4-aminosulfonyl)phenyl)-5-
methylpyrimidine-2,4-diamine;
N4-(1,3-dimethylbenzimidazolin-2-on-5-yl)-N2-((4-aminosulfonyl)phenyl)-5-
fluoropyrimidine-2,4-diamine;
N4-(1,3-dimethylbenzimidazolin-2-on-5-yl)-N2-((4-methylsulfonylamino)phenyl)-5-
methylpyrimidine-2,4-diamine;
N4-(1,3-dimethylbenzimidazolin-2-on-5-yl)-N2-((4-methylsulfonylamino)phenyl)-5-
fluoropyrimidine-2,4-diamine;
N4-(1,3-dimethylbenzimidazolin-2-on-5-yl)-N2-[2-(4-methylpiperazino)pyridin-5-yl]-5-
methylpyrimidine-2,4-diamine;
N4-(1,3-dimethylbenzimidazolin-2-on-5-yl)-N2-[2-(4-methylpiperazino)pyridin-5-yl]-5-
fluoropyrimidine-2,4-diamine;
N4-(3-isopropylbenzo[d]oxazol-2(3H)-on-5-yl)-N2-((3-methylsulfonyl)phenyl)-5-
methylpyrimidine-2,4-diamine;
N4-(3-isopropylbenzo[d]oxazol-2(3H)-on-5-yl)-N2-((4-methylsulfonylamino)phenyl)-5-
methylpyrimidine-2,4-diamine;
N4-(3-methylbenzo[d]oxazol-2(3H)-on-6-yl)-N2-((3-morpholinyl)phenyl)-5-
methylpyrimidine-2,4-diamine;
N4-(3-n-propylbenzo[d]oxazol-2(3H)-on-5-yl)-N2-((3-methylsulfonyl)phenyl)-5-
methylpyrimidine-2,4-diamine;
N4-(3-n-propylbenzo[d]oxazol-2(3H)-on-5-yl)-N2-((4-methylsulfonylamino)phenyl)-5-
methylpyrimidine-2,4-diamine;
N4-(3-n-propylbenzo[d]oxazol-2(3H)-on-5-yl)-N2-(6-(4-methylpiperazin-1-yl)pyridin-3-
yl)-5-methylpyrimidine-2,4-diamine;
N4-(3-phosphorylmethylbenzo[d]oxazol-2(3H)-on-5-yl)-N2-((3-methylsulfonyl)phenyl)-5-
methylpyrimidine-2,4-diamine;
N4-(7-chloro-2-oxo-2,3-dihydro-1,3-benzoxazol-5-yl)-5-methyl-N2-(3,4,5-
trimethyl)phenyl-2,4-pyrimidinediamine;
N4-(benzimidazolin-2-on-5-yl)-N2-((2-morpholinyl)pyridin-5-yl)-5-fluoropyrimidine-2,4-
diamine;
N4-(benzimidazolin-2-on-5-yl)-N2-((3-aminosulfonyl)phenyl)-5-methylpyrimidine-2,4-
diamine;
N4-(benzimidazolin-2-on-5-yl)-N2-((4-aminosulfonyl)phenyl)-5-methylpyrimidine-2,4-
diamine;
N4-(benzimidazolin-2-on-5-yl)-N2-(3-(morpholinosulfonyl)phenyl)-5-fluoropyrimidine-
2,4-diamine;
N4-(benzimidazolin-2-on-5-yl)-N2-(3-cyanophenyl)-5-methylpyrimidine-2,4-diamine;
N4-(benzimidazolin-2-on-5-yl)-N2-(3-methylsulfonyl)phenyl-5-methylpyrimidine-2,4-
diamine;
N4-(benzimidazolin-2-on-5-yl)-N2-(4-cyanophenyl)-5-methylpyrimidine-2,4-diamine;
N4-(benzimidazolin-2-on-5-yl)-N2-(4-methylsulfonyl)phenyl-5-fluoropyrimidine-2,4-
diamine;
N4-(benzimidazolin-2-on-5-yl)-N2-[2-(4-methylpiperazin-1-yl)pyridin-5-yl]-5-
methylpyrimidine-2,4-diamine;
N4-(benzimidazolin-2-on-5-yl)-N2-[2-(4-methylpiperazin-1-yl)pyridin-5-yl]-5-
fluoropyrimidine-2,4-diamine;
N4-(benzimidazolin-2-on-5-yl)-N2-[3-methyl-2-(4-methylpiperazin-1-yl)pyridin-5-yl]-5-
methylpyrimidine-2,4-diamine;
N4-(benzo[d]oxazol-2(3H)-on-5-yl)-N2-((2-morpholinyl)pyridin-5-yl)-5-methylpyrimidine-
2,4-diamine;
N4-(benzo[d]oxazol-2(3H)-on-5-yl)-N2-((2-morpholinyl)pyridin-5-yl)-5-fluoropyrimidine-
2,4-diamine;
N4-(benzo[d]oxazol-2(3H)-on-5-yl)-N2-((3-aminosulfonyl-4-methyl)phenyl)-5-
methylpyrimidine-2,4-diamine;
N4-(benzo[d]oxazol-2(3H)-on-5-yl)-N2-((3-morpholinyl)phenyl)-5-fluoropyrimidine-2,4-
diamine;
N4-(benzo[d]oxazol-2(3H)-on-5-yl)-N2-((3-morpholinyl)phenyl)-5-methylpyrimidine-2,4-
diamine;
N4-(benzo[d]oxazol-2(3H)-on-5-yl)-N2-((3-tert-butylaminosulfonyl)phenyl)-5-
methylpyrimidine-2,4-diamine;
N4-(benzo[d]oxazol-2(3H)-on-5-yl)-N2-((4-(1,4-diazabicyclo[3.2.2]nonan-4-yl)-3-
methyl)phenyl)-5-methylpyrimidine-2,4-diamine;
N4-(benzo[d]oxazol-2(3H)-on-5-yl)-N2-((4-(dihydro-1H-pyrido[1,2-a]pyrazin-
2(6H,7H,8H,9H,9aH)-yl)-3-methyl)phenyl)-5-methylpyrimidine-2,4-diamine;
N4-(benzo[d]oxazol-2(3H)-on-5-yl)-N2-(1,2-benzisoxazol-6-yl)-5-methylpyrimidine-2,4-
diamine;
N4-(benzo[d]oxazol-2(3H)-on-5-yl)-N2-(1H-indazol-5-yl)-5-methylpyrimidine-2,4-
diamine;
N4-(benzo[d]oxazol-2(3H)-on-5-yl)-N2-(1H-indazol-6-yl)-5-methylpyrimidine-2,4-
diamine;
N4-(benzo[d]oxazol-2(3H)-on-5-yl)-N2-(2-(bicyclo[2.2.1]heptan-2-ylmethyl)isoindolin-5-
yl)-5-methylpyrimidine-2,4-diamine;
N4-(benzo[d]oxazol-2(3H)-on-5-yl)-N2-(2-cyclopentylmethylisoindolin-5-yl)-5-
methylpyrimidine-2,4-diamine;
N4-(benzo[d]oxazol-2(3H)-on-5-yl)-N2-(2-cyclopropylmethylylisoindolin-5-yl)-5-
methylpyrimidine-2,4-diamine;
N4-(benzo[d]oxazol-2(3H)-on-5-yl)-N2-(2-ethylisoindolin-5-yl)-5-methylpyrimidine-2,4-
diamine;
N4-(benzo[d]oxazol-2(3H)-on-5-yl)-N2-(2Hydroxyisoindolin-5-yl)-5-methylpyrimidine-
2,4-diamine;
N4-(benzo[d]oxazol-2(3H)-on-5-yl)-N2-(2-isobutylisoindolin-5-yl)-5-methylpyrimidine-
2,4-diamine;
N4-(benzo[d]oxazol-2(3H)-on-5-yl)-N2-(2-isopentylisoindolin-5-yl)-5-methylpyrimidine-
2,4-diamine;
N4-(benzo[d]oxazol-2(3H)-on-5-yl)-N2-(2-methylisoindolin-5-yl)-5-methylpyrimidine-2,4-
diamine;
N4-(benzo[d]oxazol-2(3H)-on-5-yl)-N2-(2-n-propylisoindolin-5-yl)-5-methylpyrimidine-
2,4-diamine;
N4-(benzo[d]oxazol-2(3H)-on-5-yl)-N2-(2-tert-butoxoxycarbonylisoindolin-5-yl)-5-
methylpyrimidine-2,4-diamine;
N4-(benzo[d]oxazol-2(3H)-on-5-yl)-N2-(3-(morpholinosulfonyl)phenyl)-5-
methylpyrimidine-2,4-diamine;
N4-(benzo[d]oxazol-2(3H)-on-5-yl)-N2-(3-(morpholinosulfonyl)phenyl)-5-
fluoropyrimidine-2,4-diamine;
N4-(benzo[d]oxazol-2(3H)-on-5-yl)-N2-(3,4,5-trimethoxy)phenyl-5-methylpyrimidine-2,4-
diamine;
N4-(benzo[d]oxazol-2(3H)-on-5-yl)-N2-(3-aminocarbonylphenyl)-5-methylpyrimidine-2,4-
diamine;
N4-(benzo[d]oxazol-2(3H)-on-5-yl)-N2-(3-aminosulfonyl)phenyl)-5-fluoropyrimidine-2,4-
diamine;
N4-(benzo[d]oxazol-2(3H)-on-5-yl)-N2-(3-aminosulfonyl)phenyl)-5-methylpyrimidine-2,4-
diamine;
N4-(benzo[d]oxazol-2(3H)-on-5-yl)-N2-(3-cyanophenyl)-5-fluoropyrimidine-2,4-diamine;
N4-(benzo[d]oxazol-2(3H)-on-5-yl)-N2-(3-cyanophenyl)-5-methylpyrimidine-2,4-diamine;
N4-(benzo[d]oxazol-2(3H)-on-5-yl)-N2-(3-fluoro-4-(1,5,7-trimethyl-3,7-
diazabicyclo[3.3.1]nonan-3-yl)phenyl)-5-methylpyrimidine-2,4-diamine;
N4-(benzo[d]oxazol-2(3H)-on-5-yl)-N2-(3-formylphenyl)-5-methylpyrimidine-2,4-
diamine;
N4-(benzo[d]oxazol-2(3H)-on-5-yl)-N2-(3-methyl-1,2-benzisoxazol-5-yl)-5-
methylpyrimidine-2,4-diamine;
N4-(benzo[d]oxazol-2(3H)-on-5-yl)-N2-(3-methyl-4-(1,5,7-trimethyl-3,7-
diazabicyclo[3.3.1]nonan-3-yl)phenyl)-5-methylpyrimidine-2,4-diamine;
N4-(benzo[d]oxazol-2(3H)-on-5-yl)-N2-(3-methylsulfonyl)phenyl)-5-methylpyrimidine-
2,4-diamine;
N4-(benzo[d]oxazol-2(3H)-on-5-yl)-N2-(3-methylsulfonyl)phenyl)-5-fluoropyrimidine-2,4-
diamine;
N4-(benzo[d]oxazol-2(3H)-on-5-yl)-N2-(3-methylsulfonylamino)phenyl)-5-
methylpyrimidine-2,4-diamine formate salt;
N4-(benzo[d]oxazol-2(3H)-on-5-yl)-N2-(3-methylsulfonylamino)phenyl)-5-
fluoropyrimidine-2,4-diamine;
N4-(benzo[d]oxazol-2(3H)-on-5-yl)-N2-(3-trifluoromethylsulfonyl)phenyl-5-
methylpyrimidine-2,4-diamine;
N4-(benzo[d]oxazol-2(3H)-on-5-yl)-N2-(4-(8-methyl-2,8-diazabicyclo[3.2.1]octan-2-
yl)phenyl)-5-methylpyrimidine-2,4-diamine;
N4-(benzo[d]oxazol-2(3H)-on-5-yl)-N2-(4-(8-methyl-8-azabicyclo[3.2.1]octan-3-
ylamino)phenyl)-5-methylpyrimidine-2,4-diamine;
N4-(benzo[d]oxazol-2(3H)-on-5-yl)-N2-(4-aminocarbonylphenyl)-5-methylpyrimidine-2,4-
diamine;
N4-(benzo[d]oxazol-2(3H)-on-5-yl)-N2-(4-aminosulfonyl)phenyl)-5-fluoropyrimidine-2,4-
diamine;
N4-(benzo[d]oxazol-2(3H)-on-5-yl)-N2-(4-aminosulfonyl)phenyl)-5-methylpyrimidine-2,4-
diamine;
N4-(benzo[d]oxazol-2(3H)-on-5-yl)-N2-(4-cyanophenyl)-5-fluoropyrimidine-2,4-diamine;
N4-(benzo[d]oxazol-2(3H)-on-5-yl)-N2-(4-cyanophenyl)-5-methylpyrimidine-2,4-diamine;
N4-(benzo[d]oxazol-2(3H)-on-5-yl)-N2-(4-formylphenyl)-5-methylpyrimidine-2,4-
diamine;
N4-(benzo[d]oxazol-2(3H)-on-5-yl)-N2-(4-methylsulfonyl)phenyl)-5-methylpyrimidine-
2,4-diamine;
N4-(benzo[d]oxazol-2(3H)-on-5-yl)-N2-(4-methylsulfonyl)phenyl)-5-fluoropyrimidine-2,4-
diamine;
N4-(benzo[d]oxazol-2(3H)-on-5-yl)-N2-(4-methylsulfonylamino)phenyl)-5-
methylpyrimidine-2,4-diamine;
N4-(benzo[d]oxazol-2(3H)-on-5-yl)-N2-(4-methylsulfonylamino)phenyl)-5-
fluoropyrimidine-2,4-diamine;
N4-(benzo[d]oxazol-2(3H)-on-5-yl)-N2-(4-trifluoromethylsulfonyl)phenyl-5-
methylpyrimidine-2,4-diamine;
N4-(benzo[d]oxazol-2(3H)-on-5-yl)-N2-(6-((1S,4R)-5-methyl-2,5-
diazabicyclo[2.2.1]heptan-2-yl)-pyridin-3-yl)-5-methylpyrimidine-2,4-diamine;
N4-(benzo[d]oxazol-2(3H)-on-5-yl)-N2-(6-(3-methyl-4-tert-butoxycarbonylpiperazin-1-
yl)pyridin-3-yl)-5-methylpyrimidine-2,4-diamine;
N4-(benzo[d]oxazol-2(3H)-on-5-yl)-N2-(6-(3-methylpiperazin-1-yl)pyridin-3-yl)-5-
methylpyrimidine-2,4-diamine;
N4-(benzo[d]oxazol-2(3H)-on-5-yl)-N2-(6-(4-methoxycarbonylpiperazin-1-yl)pyridin-3-
yl)-5-methylpyrimidine-2,4-diamine;
N4-(benzo[d]oxazol-2(3H)-on-5-yl)-N2-(6-(4-methyl-1,4-diazepan-1-yl)pyridin-3-yl)-5-
methylpyrimidine-2,4-diamine;
N4-(benzo[d]oxazol-2(3H)-on-5-yl)-N2-(6-(4-methylpiperazin-1-yl)pyridazin-3-yl)-5-
methylpyrimidine-2,4-diamine;
N4-(benzo[d]oxazol-2(3H)-on-5-yl)-N2-(6-(4-methylpiperidin-1-yl)pyridin-3-yl)-5-
methylpyrimidine-2,4-diamine;
N4-(benzo[d]oxazol-2(3H)-on-5-yl)-N2-(6-(4-tert-butyloxycarbonylpiperazin-1-
yl)pyridin-3-yl)-5-methylpyrimidine-2,4-diamine;
N4-(benzo[d]oxazol-2(3H)-on-5-yl)-N2-(6-(4-trifluoromethoxycarbonylpiperazin-1-
yl)pyridin-3-yl)-5-methylpyrimidine-2,4-diamine;
N4-(benzo[d]oxazol-2(3H)-on-5-yl)-N2-(6-(piperazin-1-yl)pyridin-3-yl)-5-
methylpyrimidine-2,4-diamine;
N4-(benzo[d]oxazol-2(3H)-on-5-yl)-N2-(6,7,8,9-tetrahydro-5H-benzo[7]annulen-5-on-3-
yl)-5-methylpyrimidine-2,4-diamine;
N4-(benzo[d]oxazol-2(3H)-on-5-yl)-N2-(6-dimethylaminopyridin-3-yl)-5-
methylpyrimidine-2,4-diamine;
N4-(benzo[d]oxazol-2(3H)-on-5-yl)-N2-(7-(pyrrolidin-1-yl)-6,7,8,9-tetrahydro-5H-
benzo[7]annulen-2-yl)-5-methylpyrimidine-2,4-diamine;
N4-(benzo[d]oxazol-2(3H)-on-5-yl)-N2-(isoindolin-5-yl)-5-methylpyrimidine-2,4-diamine;
N4-(benzo[d]oxazol-2(3H)-on-5-yl)-N2-[2-(1H-piperidin-4-yl)aminopyridin-5-yl]-5-
methylpyrimidine-2,4-diamine;
N4-(benzo[d]oxazol-2(3H)-on-5-yl)-N2-[2-(1-methylpiperidin-4-yl)aminopyridin-5-yl]-5-
methylpyrimidine-2,4-diamine;
N4-(benzo[d]oxazol-2(3H)-on-5-yl)-N2-[2-(4-methylpiperazin-1-yl)pyridin-5-yl]-5-
fluoropyrimidine-2,4-diamine;
N4-(benzo[d]oxazol-2(3H)-on-5-yl)-N2-[2-(4-methylpiperazino)pyridin-4-yl]-5-
methylpyrimidine-2,4-diamine;
N4-(benzo[d]oxazol-2(3H)-on-5-yl)-N2-[2-(8-methyl-8-aza-bicyclo[3.2.1]oct-3-
yl)aminopyridin-5-yl]-5-methylpyrimidine-2,4-diamine;
N4-(benzo[d]oxazol-2(3H)-on-5-yl)-N2-[2-(piperazino)pyridin-4-yl]-5-methylpyrimidine-
2,4-diamine;
N4-(benzo[d]oxazol-2(3H)-on-6-yl)-N2-((3-morpholinyl)phenyl)-5-methylpyrimidine-2,4-
diamine;
N4-(benzo[d]oxazolin-2(3H)-on-5-yl)-N2-[3-trifluoromethyl-2-(4-methylpiperazin-1-
yl)pyridin-5-yl]-5-methylpyrimidine-2,4-diamine;
N4-(benzo[d]oxazolin-2(3H)-one-5-yl)-5-methyl-N2-[2-((1S,4S)-5-methyl-2,5-
diazabicyclo[2.2.1]heptan-2-yl)-3-trifluoromethylpyridine-5-yl]-2,4-pyrimidinediamine;
N4-(benzo[d]oxazolin-2(3H)-one-5-yl)-5-methyl-N2-[3-(morpholin-4-yl)-4-
trifluoromethoxyphenyl]-2,4-pyrimidinediamine;
N4-(benzo[d]oxazolin-2(3H)-one-5-yl)-N2-[2-(4-ethylpiperazin-1-yl)-3-
trifluoromethylpyridine-5-yl]-5-methyl-2,4-pyrimidinediamine;
N4-(benzo[d]oxazolin-2(3H)-one-5-yl)-N2-[2-(4-ethylpiperazin-1-yl)-3-fluoropyridine-5-
yl]-5-methyl-2,4-pyrimidinediamine;
N4-(benzo[d]oxazolin-2(3H)-one-5-yl)-N2-[3-cyano-2-((1S,4S)-5-methyl-2,5-
diazabicyclo[2.2.1]heptan-2-yl)pyridine-5-yl]-5-methyl-2,4-pyrimidinediamine;
N4-(benzo[d]oxazolin-2(3H)-one-5-yl)-N2-[3-fluoro-2-(4-methylpiperazin-1-yl)pyridine-5-
yl]-5-methyl-2,4-pyrimidinediamine;
N4-(benzo[d]oxazolin-2(3H)-one-5-yl)-N2-{2-[(8R)-1,4-diazabicyclo[4.3.0]nonane-1-yl]-
3-fluoropyridine-5-yl}-5-methyl-2,4-pyrimidinediamine;
N4-(benzo[d]oxazolin-2(3H)-one-5-yl)-N2-{2-[(8S)-1,4-diazabicyclo[4.3.0]nonane-1-yl]-3-
fluoropyridine-5-yl}-5-methyl-2,4-pyrimidinediamine;
N4-(benzoxazolin-2-on-5-yl)-N2-(3,4,5-trimethoxyphenyl)-5-fluoropyrimidine-2,4-
diamine;
N4-(benzoxazolin-2-on-5-yl)-N2-(3-trifluoromethoxyphenyl)-5-fluoropyrimidine-2,4-
diamine trifluoroacetate salt;
N4-(benzoxazolin-2-on-5-yl)-N2-(3-trifluoromethoxyphenyl)-5-methylpyrimidine-2,4-
diamine trifluoroacetate salt;
N4-(benzoxazolin-2-on-5-yl)-N2-(4-trifluoromethoxyphenyl)-5-fluoropyrimidine-2,4-
diamine trifluoroacetate salt;
N4-(benzoxazolin-2-on-5-yl)-N2-(4-trifluoromethoxyphenyl)-5-methylpyrimidine-2,4-
diamine trifluoroacetate salt;
N4-(benzoxazolin-2-on-5-yl)-N2-[2-((1S,4S)-2-oxa-5-azabicyclo[2.2.1]heptan-5-yl)pyridin-
5-yl]-5-methylpyrimidine-2,4-diamine;
N4-(benzoxazolin-2-on-5-yl)-N2-[2-((1S,4S)-2-oxa-5-azabicyclo[2.2.1]heptan-5-yl)pyridin-
5-yl]-5-fluoropyrimidine-2,4-diamine;
N4-(benzoxazolin-2-on-5-yl)-N2-[2-((1S,4S)-5-methyl-2,5-diazabicyclo[2.2.1]heptan-2-
yl)pyridin-5-yl]-5-methylpyrimidine-2,4-diamine;
N4-(benzoxazolin-2-on-5-yl)-N2-[2-((1S,4S)-5-methyl-2,5-diazabicyclo[2.2.1]heptan-2-
yl)pyridin-5-yl]-5-fluoropyrimidine-2,4-diamine;
N4-(benzoxazolin-2-on-5-yl)-N2-[2-(4-methylpiperazin-1-yl)pyridin-5-yl]-5-
methylpyrimidine-2,4-diamine;
N4-(benzoxazolin-2-on-5-yl)-N2-[3-fluoro-2-((1S,4S)-5-methyl-2,5-
diazabicyclo[2.2.1]heptan-2-yl)pyridin-5-yl]-5-methylpyrimidine-2,4-diamine;
N4-(benzoxazolin-2-on-5-yl)-N2-[3-methyl-2-((1S,4S)-5-methyl-2,5-
diazabicyclo[2.2.1]heptan-2-yl)pyridin-5-yl]-5-methylpyrimidine-2,4-diamine;
N4-(benzoxazolin-2-on-5-yl)-N2-[3-methyl-2-((1S,4S)-5-methyl-2,5-
diazabicyclo[2.2.1]heptan-2-yl)pyridin-5-yl]-5-fluoropyrimidine-2,4-diamine;
N4-(benzoxazolin-2-on-5-yl)-N2-[3-methyl-2-(4-methylpiperazin-1-yl)pyridin-5-yl]-5-
methylpyrimidine-2,4-diamine;
N4-(benzoxazolin-2-on-5-yl)-N2-[3-methyl-2-(4-methylpiperazin-1-yl)pyridin-5-yl]-5-
fluoropyrimidine-2,4-diamine;
N4-(benzoxazolin-2-on-5-yl)-N2-[3-methyl-4-(4-methylpiperazin-1-yl)phenyl]-5-
methylpyrimidine-2,4-diamine;
N4-(benzoxazolin-2-on-5-yl)-N2-[3-N-(i-propyl)aminosulfonylphenyl]-5-
methylpyrimidine-2,4-diamine;
N4-(benzoxazolin-2-on-5-yl)-N2-[3-trifluoromethyl-4-(4-ethylpiperazin-1-yl)phenyl]-5-
methylpyrimidine-2,4-diamine;
N4-(benzoxazolin-2-on-5-yl)-N2-[4-methyl-3-N-(t-butyl)aminosulfonylphenyl]-5-
methylpyrimidine-2,4-diamine;
N4-(benzoxazolin-2-on-5-yl)-N2-[4-N-(t-butyl)aminosulfonylphenyl]-5-methylpyrimidine-
2,4-diamine;
N4-[7-chloro-3-(phosphonooxy)methyl-2-oxo-2,3-dihydro-1,3-benzoxazol-5-yl]-5-methyl-
N2-(3,4,5-trimethyl)phenyl-2,4-pyrimidinediamine;
N4-[7-chloro-3-(phosphonooxy)methyl-2-oxo-2,3-dihydro-1,3-benzoxazol-5-yl]-5-methyl-
N2-(3,4,5-trimethyl)phenyl-2,4-pyrimidinediamine bis-sodium salt;
N4-{3-[2-(N,N-dimethylamino)ethoxy]phosphinyloxymethyl-2-oxo-2,3-dihydro-1,3-
benzoxazol-5-yl}-5-methyl-N2-(3,4,5-trimethyl)phenyl-2,4-pyrimidinediamine;
N4-{3-[bis(1,1-dimethylethoxy)]phosphinyloxymethyl-2-oxo-2,3-dihydro-1,3-benzoxazol-
5-yl}-N2-(3-methoxy-5-trifluoromethyl)phenyl-5-methyl-2,4-pyrimidinediamine;
N4-{3-[bis(1,1-dimethylethoxy)]phosphinyloxymethyl-2-oxo-2,3-dihydro-1,3-benzoxazol-
5-yl}-N2-(3,4,5-trimethyl)phenyl-5-methyl-2,4-pyrimidinediamine;
N4-{3-[bis(1,1-dimethylethoxy)]phosphinyloxymethyl-2-oxo-2,3-dihydro-1,3-benzoxazol-
5-yl}-N2-(3,5-dimethyl-4-fluoro)phenyl-5-methyl-2,4-pyrimidinediamine;
N4-{3-[bis(1,1-dimethylethoxy)]phosphinyloxymethyl-7-chloro-2-oxo-2,3-dihydro-1,3-
benzoxazol-5-yl}-N2-(3,4,5-trimethyl)phenyl-5-methyl-2,4-pyrimidinediamine;
N4-{3-bis[2-(N,N-dimethylamino)ethoxy]phosphinyloxymethyl-2-oxo-2,3-dihydro-1,3-
benzoxazol-5-yl}-5-methyl-N2-(3,4,5-trimethyl)phenyl-2,4-pyrimidinediamine;
N-Cyclobutyl-2-methyl-4-[5-methyl-4-(2-oxo-2,3-dihydro-benzooxazol-5-ylamino)-
pyrimidin-2-ylamino]-benzamide;
N-Cyclobutyl-3-[5-methyl-4-(2-oxo-2,3-dihydro-benzooxazol-5-ylamino)-pyrimidin-2-
ylamino]-5-trifluoromethyl-benzamide;
N-cyclobutyl-4-(5-fluoro-4-(2-oxo-2,3-dihydrobenzo[d]oxazol-5-ylamino)pyrimidin-2-
ylamino)benzamide formate salt;
N-cyclobutyl-4-(5-fluoro-4-(2-oxo-2,3-dihydrobenzo[d]oxazol-5-ylamino)pyrimidin-2-
ylamino)-2-methoxybenzamide formate salt;
N-cyclobutyl-4-(5-methyl-4-(2-oxo-2,3-dihydrobenzo[d]oxazol-5-ylamino)pyrimidin-2-
ylamino)-2-(trifluoromethyl)benzamide;
N-Cyclobutyl-4-[5-methyl-4-(2-oxo-2,3-dihydro-benzooxazol-5-ylamino)-pyrimidin-2-
ylamino]-benzamide formic acid salt;
N-Cyclobutyl-4-[5-methyl-4-(2-oxo-2,3-dihydro-benzooxazol-5-ylamino)-pyrimidin-2-
ylamino]-benzamide;
N-cyclopropyl-2-methoxy-4-(5-methyl-4-(2-oxo-2,3-dihydrobenzo[d]oxazol-5-
ylamino)pyrimidin-2-ylamino)benzamide;
N-cyclopropyl-4-(5-fluoro-4-(2-oxo-2,3-dihydrobenzo[d]oxazol-5-ylamino)pyrimidin-2-
ylamino)benzamide formate salt;
N-cyclopropyl-4-(5-fluoro-4-(2-oxo-2,3-dihydrobenzo[d]oxazol-5-ylamino)pyrimidin-2-
ylamino)-2-(trifluoromethyl)benzamide formate salt;
N-cyclopropyl-4-(5-methyl-4-(2-oxo-2,3-dihydrobenzo[d]oxazol-5-ylamino)pyrimidin-2-
ylamino)-2-(trifluoromethyl)benzamide;
N-Cyclopropyl-4-[5-methyl-4-(2-oxo-2,3-dihydro-benzooxazol-5-ylamino)-pyrimidin-2-
ylamino]-benzamide formate salt;
N-Cyclopropyl-4-[5-methyl-4-(2-oxo-2,3-dihydro-benzooxazol-5-ylamino)-pyrimidin-2-
ylamino]-benzamide trifluoroacetic acid salt;
N-Cyclopropyl-4-[5-methyl-4-(2-oxo-2,3-dihydro-benzooxazol-5-ylamino)-pyrimidin-2-
ylamino]-benzamide;
N-Ethyl-2-methyl-4-[5-methyl-4-(2-oxo-2,3-dihydro-benzooxazol-5-ylamino)-pyrimidin-2-
ylamino]-benzamide;
N-ethyl-4-(5-fluoro-4-(2-oxo-2,3-dihydrobenzo[d]oxazol-5-ylamino)pyrimidin-2-
ylamino)benzamide formate salt;
N-Ethyl-4-[5-methyl-4-(2-oxo-2,3-dihydro-benzooxazol-5-ylamino)-pyrimidin-2-ylamino]-
benzamide;
N-Isopropyl-4-[5-methyl-4-(2-oxo-2,3-dihydro-benzooxazol-5-ylamino)-pyrimidin-2-
ylamino]-benzamide;
N-Methyl-4-[5-methyl-4-(2-oxo-2,3-dihydro-benzooxazol-5-ylamino)-pyrimidin-2-
ylamino]-benzamide;
Sodium(5-(2-(3-methoxy-5-methylphenylamino)-5-methylpyrimidin-4-ylamino)-7-methyl-
2-oxobenzo[d]oxazol-3(2H)-yl)methyl phosphate;
sodium(5-(2-(4-(cyclobutylcarbamoyl)phenylamino)-5-methylpyrimidin-4-ylamino)-2-
oxobenzo[d]oxazol-3(2H)-yl)methyl phosphate;
sodium(5-(2-(4-carbamoylphenylamino)-5-methylpyrimidin-4-ylamino)-2-
oxobenzo[d]oxazol-3(2H)-yl)methyl phosphate;
Sodium(5-(2-(4-fluoro-3-methoxy-5-methylphenylamino)-5-methylpyrimidin-4-ylamino)-
2-oxobenzo[d]oxazol-3(2H)-yl)methyl phosphate;
tert-butyl 1-(5-(5-methyl-4-(2-oxo-2,3-dihydrobenzo[d]oxazol-5-ylamino)pyrimidin-2-
ylamino)pyridin-2-yl)pyrrolidin-3-ylcarbamate;
tert-butyl 3-(3-methyl-5-(5-methyl-4-(2-oxo-2,3-dihydrobenzo[d]oxazol-5-
ylamino)pyrimidin-2-ylamino)pyridin-2-yl)-8-azabicyclo[3.2.1]octane-8-carboxylate;
4-(6-(dimethylamino)pyridin-3-yl)-N-(6-((1S,4S)-5-methyl-2,5-diazabicyclo[2.2.1]heptan-
2-yl)pyridin-3-yl)pyrimidin-2-amine;
4-(6-(methylcarbonylamino)pyridin-3-yl)-N-(4-((1S,4S)-5-methyl-2,5-
diazabicyclo[2.2.1]heptan-2-yl)pyridin-3-yl)pyrimidin-2-amine;
4-(4-(dimethylamino)phenyl)-N-(6-((1S,4S)-5-methyl-2,5-diazabicyclo[2.2.1]heptan-2-
yl)pyridin-3-yl)pyrimidin-2-amine;
4-(6-(4-acetylpiperazin-1-yl)pyridin-3-yl)-N-(6-((1S,4S)-5-methyl-2,5-
diazabicyclo[2.2.1]heptan-2-yl)pyridin-3-yl)pyrimidin-2-amine;
4-(5-methyl-6-(morpholin-4-yl)pyridin-3-yl)-N-(6-((1S,4S)-5-methyl-2,5-
diazabicyclo[2.2.1]heptan-2-yl)pyridin-3-yl)pyrimidin-2-amine;
4-(6-(dimethylamino)pyridin-3-yl)-N-(5-methyl-6-((1S,4S)-5-methyl-2,5-
diazabicyclo[2.2.1]heptan-2-yl)pyridin-3-yl)pyrimidin-2-amine;
4-(4-(dimethylamino)phenyl)-N-(5-methyl-6-((1S,4S)-(5-methyl-2,5-
diazabicyclo[2.2.1]heptan-2-yl)pyridin-3-yl)pyrimidin-2-amine;
4-(6-(4-acetylpiperazin-1-yl)pyridin-3-yl)-N-(5-methyl-6-((1S,4S)-5-methyl-2,5-
diazabicyclo[2.2.1]heptan-2-yl)pyridin-3-yl)pyrimidin-2-amine;
4-(5-methyl-6-(morpholin-4-yl)pyridin-3-yl)-N-(5-methyl-6-((1S,4S)-5-methyl-2,5-
diazabicyclo[2.2.1]heptan-2-yl)pyridin-3-yl)pyrimidin-2-amine;
4-(4-methyl-3,4-dihydro-2H-pyrido[3,2-b][1,4]oxazin-7-yl)-N-(5-methyl-6-((1S,4S)-5-
methyl-2,5-diazabicyclo[2.2.1]heptan-2-yl)pyridin-3-yl)pyrimidin-2-amine;
4-(6-(morpholin-4-yl)pyridin-3-yl)-N-(5-methyl-6-((1S,4S)-5-methyl-2,5-
diazabicyclo[2.2.1]heptan-2-yl)pyridin-3-yl)pyrimidin-2-amine;
4-(4-(t-butylcarbonylamino)phenyl)-N-(5-methyl-6-((1S,4S)-5-methyl-2,5-
diazabicyclo[2.2.1]heptan-2-yl)pyridin-3-yl)pyrimidin-2-amine;
4-(2,2-dimethyl-3-oxo-3,4-dihydro-2H-pyrido[3,2-b][1,4]oxazin-7-yl)-N-(5-methyl-6-
((1S,4S)-5-methyl-2,5-diazabicyclo[2.2.1]heptan-2-yl)pyridin-3-yl)pyrimidin-2-amine;
4-(4-(3-cyclopropylureido)phenyl)-N-(5-methyl-6-((1S,4S)-5-methyl-2,5-
diazabicyclo[2.2.1]heptan-2-yl)pyridin-3-yl)pyrimidin-2-amine;
4-(6-(2-(morpholin-4-yl)acetamido)pyridin-3-yl)-N-(5-methyl-6-((1S,4S)-5-methyl-2,5-
diazabicyclo[2.2.1]heptan-2-yl)pyridin-3-yl)pyrimidin-2-amine;
4-(6-aminopyridin-3-yl)-N-(5-methyl-6-((1S,4S)-5-methyl-2,5-diazabicyclo[2.2.1]heptan-2-
yl)pyridin-3-yl)pyrimidin-2-amine;
4-(6-(acetamido)pyridin-3-yl)-N-(5-methyl-6-((1S,4S)-5-methyl-2,5-
diazabicyclo[2.2.1]heptan-2-yl)pyridin-3-yl)pyrimidin-2-amine;
4-(6-(methylsulfonylamino)pyridin-3-yl)-N-(5-methyl-6-((1S,4S)-5-methyl-2,5-
diazabicyclo[2.2.1]heptan-2-yl)pyridin-3-yl)pyrimidin-2-amine;
4-(2-(dimethylamino)thiazol-4-yl)-N-(6-((1S,4S)-5-methyl-2,5-diazabicyclo[2.2.1]heptan-2-
yl)pyridin-3-yl)pyrimidin-2-amine;
4-(5-(morpholin-4-yl)pyrazin-2-yl)-N-(6-((1S,4S)-5-methyl-2,5-diazabicyclo[2.2.1]heptan-
2-yl)pyridin-3-yl)pyrimidin-2-amine;
4-(1-(pyridin-4-yl)-1H-indol-5-yl)-N-(5-methyl-6-((1S,4S)-5-methyl-2,5-
diazabicyclo[2.2.1]heptan-2-yl)pyridin-3-yl)pyrimidin-2-amine;
4-(6-((1S,4S)-2-oxa-5-azabicyclo[2.2.1]heptan-5-yl)pyridin-3-yl)-N-(5-methyl-6-((1S,4S)-5-
methyl-2,5-diazabicyclo[2.2.1]heptan-2-yl)pyridin-3-yl)pyrimidin-2-amine;
4-(6-(N,N-dimethylamino)pyridin-3-yl)-N-(3-methyl-4-((1S,4S)-5-methyl-2,5-
diazabicyclo[2.2.1]heptan-2-yl)phenyl)pyrimidin-2-amine;
4-(6-(N,N-dimethylamino)pyridin-3-yl)-5-methyl-N-(3-methyl-4-((1S,4S)-5-methyl-2,5-
diazabicyclo[2.2.1]heptan-2-yl)phenyl)pyrimidin-2-amine;
4-(6-(N,N-dimethylamino)pyridin-3-yl)-5-trifluoromethyl-N-(3-methyl-4-((1S,4S)-5-
methyl-2,5-diazabicyclo[2.2.1]heptan-2-yl)phenyl)pyrimidin-2-amine;
4-(6-(N,N-dimethylamino)pyridin-3-yl)-5-fluoro-N-(3-methyl-4-((1S,4S)-5-methyl-2,5-
diazabicyclo[2.2.1]heptan-2-yl)phenyl)pyrimidin-2-amine;
4-(6-(N,N-dimethylamino)pyridin-3-yl)-5-fluoro-N-(3-fluoro-4-((1S,4S)-5-methyl-2,5-
diazabicyclo[2.2.1]heptan-2-yl)phenyl)pyrimidin-2-amine;
4-(6-(N,N-dimethylamino)pyridin-3-yl)-5-methyl-N-(3-fluoro-4-((1S,4S)-5-methyl-2,5-
diazabicyclo[2.2.1]heptan-2-yl)phenyl)pyrimidin-2-amine;
4-(6-(N,N-dimethylamino)pyridin-3-yl)-N-(3-fluoro-4-((1S,4S)-5-methyl-2,5-
diazabicyclo[2.2.1]heptan-2-yl)phenyl)pyrimidin-2-amine;
4-(4-methyl-3,4-dihydro-2H-pyrido[3,2-b][1,4]oxazin-7-yl)-N-(3-methyl-4-((1S,4S)-5-
methyl-2,5-diazabicyclo[2.2.1]heptan-2-yl)phenyl)pyrimidin-2-amine;
4-(4-methyl-3,4-dihydro-2H-pyrido[3,2-b][1,4]oxazin-7-yl)-N-(3-fluoro-4-((1S,4S)-5-
methyl-2,5-diazabicyclo[2.2.1]heptan-2-yl)phenyl)pyrimidin-2-amine;
4-(6-(morpholin-4-yl)pyridin-3-yl)-N-(3-methyl-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-fluoro-4-((1S,4S)-5-methyl-2,5-
diazabicyclo[2.2.1]heptan-2-yl)phenyl)pyrimidin-2-amine;
4-(6-((1S,4S)-5-methyl-2,5-diazabicyclo[2.2.1]heptan-2-yl)pyridin-3-yl)-N-(3-methyl-4-(4-
methylpiperazin-1-yl)phenyl)pyrimidin-2-amine;
4-(6-((1S,4S)-5-(4-fluorophenyl)-2,5-diazabicyclo[2.2.1]heptan-2-yl)pyridin-3-yl)-N-(3-
methyl-4-(4-methylpiperazin-1-yl)phenyl)pyrimidin-2-amine;
4-(4-(N,N-dimethylamino)phenyl)-N-(3-methyl-4-((1S,4S)-5-methyl-2,5-
diazabicyclo[2.2.1]heptan-2-yl)phenyl)pyrimidin-2-amine;
4-(4-(N,N-dimethylamino)phenyl)-N-(3-fluoro-4-((1S,4S)-5-methyl-2,5-
diazabicyclo[2.2.1]heptan-2-yl)phenyl)pyrimidin-2-amine;
4-(6-(4-acetylpiperazin-1-yl)pyridin-3-yl)-N-(3-fluoro-4-((1S,4S)-5-methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-fluoro-4-
((1S,4S)-5-methyl-2,5-diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-methyl-2,5-diazabicyclo[2.2.1]heptan-2-yl)phenyl)pyrimidin-2-amine;
4-(1H-indol-6-yl)-N-(3-methyl-4-((1S,4S)-5-methyl-2,5-diazabicyclo[2.2.1]heptan-2-
yl)phenyl)pyrimidin-2-amine;
4-(1H-pyrrolo[2,3-b]pyridin-5-yl)-N-(3-methyl-4-((1S,4S)-5-methyl-2,5-
diazabicyclo[2.2.1]heptan-2-yl)phenyl)pyrimidin-2-amine;
4-(1H-pyrrolo[2,3-b]pyridin-5-yl)-N-(3-fluoro-4-((1S,4S)-5-methyl-2,5-
diazabicyclo[2.2.1]heptan-2-yl)phenyl)pyrimidin-2-amine;
4-(6-(dimethylamino)pyridin-3-yl)-N-(3-methyl-4-((1S,4S)-5-ethyl-2,5-
diazabicyclo[2.2.1]heptan-2-yl)phenyl)pyrimidin-2-amine;
4-(6-(morpholin-4-yl)pyridin-3-yl)-N-(3-methyl-4-((1S,4S)-5-ethyl-2,5-
diazabicyclo[2.2.1]heptan-2-yl)phenyl)pyrimidin-2-amine;
4-(6-(morpholin-4-yl)pyridin-3-yl)-N-(3-methyl-4-((1S,4S)-5-((ethylamino)carbonyl)-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-oxa-2-azabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-(ethylcarbonyl)-2,5-diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-(methylsulfonyl)-2,5-diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-ethyl-2,5-diazabicyclo[2.2.1]heptan-2-yl)phenyl)pyrimidin-2-amine;
4-(3′-oxo-3′,4′-dihydrospiro[cyclobutane-1,2′-pyrido[3,2-b][1,4]oxazine]-7′-yl)-N-(3-
methyl-4-((1S,4S)-5-methyl-2,5-diazabicyclo[2.2.1]heptan-2-yl)phenyl)pyrimidin-2-amine;
4-(3′-oxo-3′,4′-dihydrospiro[cyclobutane-1,2′-pyrido[3,2-b][1,4]oxazine]-7′-yl)-N-(3-
methyl-4-((1S,4S)-5-ethyl-2,5-diazabicyclo[2.2.1]heptan-2-yl)phenyl)pyrimidin-2-amine;
4-(1H-pyrrolo[2,3-b]pyridin-5-yl)-N-(3-methyl-4-((1S,4S)-5-oxa-2-
azabicyclo[2.2.1]heptan-2-yl)phenyl)pyrimidin-2-amine;
4-(1H-pyrrolo[2,3-b]pyridin-5-yl)-N-(3-methyl-4-((1S,4S)-5-(2,2,2-trifluoroethyl)-2,5-
diazabicyclo[2.2.1]heptan-2-yl)phenyl)pyrimidin-2-amine;
4-(1H-pyrrolo[2,3-b]pyridin-5-yl)-N-(3-methyl-4-((1S,4S)-5-(cyclopropyl)methyl-2,5-
diazabicyclo[2.2.1]heptan-2-yl)phenyl)pyrimidin-2-amine;
4-(6-(morpholin-4-yl)pyridin-3-yl)-N-(3-methyl-4-((1S,4S)-5-(2,2,2-trifluoroethyl)-2,5-
diazabicyclo[2.2.1]heptan-2-yl)phenyl)pyrimidin-2-amine;
4-(6-(morpholin-4-yl)pyridin-3-yl)-N-(3-methyl-4-((1S,4S)-5-(cyclopropyl)methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-(aminosulfonyl)-2,5-diazabicyclo[2.2.1]heptan-2-yl)phenyl)pyrimidin-2-amine;
4-(3-fluoro-2-(morpholin-4-yl)pyridin-4-yl)-N-(3-fluoro-4-((1S,4S)-5-methyl-2,5-
diazabicyclo[2.2.1]heptan-2-yl)phenyl)pyrimidin-2-amine;
4-(2-(morpholin-4-yl)pyrimidin-5-yl)-N-(4-((1S,4S)-5-methyl-2,5-
diazabicyclo[2.2.1]heptan-2-yl)phenyl)pyrimidin-2-amine;
4-(2-(morpholin-4-yl)pyrimidin-5-yl)-N-(3-methyl-4-((1S,4S)-5-methyl-2,5-
diazabicyclo[2.2.1]heptan-2-yl)phenyl)pyrimidin-2-amine;
4-(2-(morpholin-4-yl)pyrimidin-5-yl)-N-(3-methyl-4-((1S,4S)-5-oxa-2-
azabicyclo[2.2.1]heptan-2-yl)phenyl)pyrimidin-2-amine;
4-(2-(morpholin-4-yl)pyrimidin-5-yl)-N-(3-fluoro-4-((1S,4S)-5-methyl-2,5-
diazabicyclo[2.2.1]heptan-2-yl)phenyl)pyrimidin-2-amine;
4-(2-((cyclopropyl)carbonylamino)-pyrimidin-5-yl)-N-(3-methyl-4-((1S,4S)-5-methyl-2,5-
diazabicyclo[2.2.1]heptan-2-yl)phenyl)pyrimidin-2-amine;
4-(4-(4,5-dihydrothiazol-2-ylcarbamoyl)phenyl)-N-(3-methyl-4-((1S,4S)-5-methyl-2,5-
diazabicyclo[2.2.1]heptan-2-yl)phenyl)pyrimidin-2-amine;
4-(4-(1,1-dimethylethyl)phenyl)-N-(3-methyl-4-((1S,4S)-5-methyl-2,5-
diazabicyclo[2.2.1]heptan-2-yl)phenyl)pyrimidin-2-amine;
4-(4-(morpholin-4-yl)phenyl)-N-(3-fluoro-4-((1S,4S)-5-methyl-2,5-
diazabicyclo[2.2.1]heptan-2-yl)phenyl)pyrimidin-2-amine TFA salt;
4-(4-((methyl)aminocarbonylmethyl)-phenyl)-N-(3-methyl-4-((1S,4S)-5-methyl-2,5-
diazabicyclo[2.2.1]heptan-2-yl)phenyl)pyrimidin-2-amine TFA salt;
4-(4-((cyclopropyl)aminocarbonyl-methyl)phenyl)-N-(3-methyl-4-((1S,4S)-5-methyl-2,5-
diazabicyclo[2.2.1]heptan-2-yl)phenyl)pyrimidin-2-amine;
4-(6-(methylaminocarbonyl)pyridin-3-yl)-N-(3-methyl-4-((1S,4S)-5-methyl-2,5-
diazabicyclo[2.2.1]heptan-2-yl)phenyl)pyrimidin-2-amine;
4-(7-oxo-5,6,7,8-tetrahydro-1,8-naphthyridin-3-yl)-N-(3-methyl-4-((1S,4S)-5-methyl-2,5-
diazabicyclo[2.2.1]heptan-2-yl)phenyl)pyrimidin-2-amine TFA salt;
4-(5-((morpholin-4-yl)carbonyl)pyridin-3-yl)-N-(3-methyl-4-((1S,4S)-5-methyl-2,5-
diazabicyclo[2.2.1]heptan-2-yl)phenyl)pyrimidin-2-amine Bis TFA salt;
4-(2,2-dimethyl-3-oxo-3,4-dihydro-2H-pyrido[3,2-b][1,4]oxazin-7-yl)-N-(3-cyano-4-
((1S,4S)-5-methyl-2,5-diazabicyclo[2.2.1]heptan-2-yl)phenyl)pyrimidin-2-amine;
4-(5-methyl-6-(morpholin-4-yl)pyridin-3-yl)-N-(3-fluoro-4-((1S,4S)-5-methyl-2,5-
diazabicyclo[2.2.1]heptan-2-yl)phenyl)pyrimidin-2-amine;
4-(5-methyl-6-(morpholin-4-yl)pyridin-3-yl)-N-(3-methyl-4-((1S,4S)-5-methyl-2,5-
diazabicyclo[2.2.1]heptan-2-yl)phenyl)pyrimidin-2-amine;
5-methyl-4-(6-(morpholin-4-yl)pyridin-3-yl)-N-(3-methyl-4-((1S,4S)-5-methyl-2,5-
diazabicyclo[2.2.1]heptan-2-yl)phenyl)pyrimidin-2-amine;
5-methyl-4-(6-(morpholin-4-yl)pyridin-3-yl)-N-(3-fluoro-4-((1S,4S)-5-methyl-2,5-
diazabicyclo[2.2.1]heptan-2-yl)phenyl)pyrimidin-2-amine;
4-(6-(2-(morpholin-4-yl)ethyl)aminopyridin-3-yl)-N-(4-((1S,4S)-5-methyl-2,5-
diazabicyclo[2.2.1]heptan-2-yl)phenyl)pyrimidin-2-amine;
4-(6-(2-(morpholin-4-yl)ethyl)aminopyridin-3-yl)-N-(3-fluoro-4-((1S,4S)-5-methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1R,4R)-5-methyl-2,5-diazabicyclo[2.2.1]heptan-2-yl)phenyl)pyrimidin-2-amine;
4-(6-(morpholin-4-yl)pyridin-3-yl)-N-(3-methyl-4-((1R,4R)-5-methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-fluoro-4-
((1R,4R)-5-methyl-2,5-diazabicyclo[2.2.1]heptan-2-yl)phenyl)pyrimidin-2-amine;
4-(6-(morpholin-4-yl)pyridin-3-yl)-N-(3-fluoro-4-((1R,4R)-5-methyl-2,5-
diazabicyclo[2.2.1]heptan-2-yl)phenyl)pyrimidin-2-amine;
4-(6-(morpholin-4-yl)pyridin-3-yl)-N-(3-methyl-4-(1,5,7-trimethyl-3,7-
diazabicyclo[3.3.1]nonan-3-yl)phenyl)pyrimidin-2-amine;
4-(6-(dimethylamino)pyridin-3-yl)-N-(3-methyl-4-(1,5,7-trimethyl-3,7-
diazabicyclo[3.3.1]nonan-3-yl)phenyl)pyrimidin-2-amine;
4-(6-(cyclohexylamino)pyridin-3-yl)-N-(3-methyl-4-(1,5,7-trimethyl-3,7-
diazabicyclo[3.3.1]nonan-3-yl)phenyl)pyrimidin-2-amine;
4-(6-(cyclohexylamino)pyridin-3-yl)-N-(3-methyl-4-((1S,4S)-5-methyl-2,5-
diazabicyclo[2.2.1]heptan-2-yl)phenyl)pyrimidin-2-amine;
4-(6-(cyclohexylamino)pyridin-3-yl)-N-(3-fluoro-4-((1S,4S)-5-methyl-2,5-
diazabicyclo[2.2.1]heptan-2-yl)phenyl)pyrimidin-2-amine;
4-(6-(benzyl)pyridin-3-yl)-N-(3-methyl-4-((1S,4S)-5-methyl-2,5-diazabicyclo[2.2.1]heptan-
2-yl)phenyl)pyrimidin-2-amine;
4-(6-(benzyl)pyridin-3-yl)-N-(3-fluoro-4-((1S,4S)-5-methyl-2,5-diazabicyclo[2.2.1]heptan-
2-yl)phenyl)pyrimidin-2-amine;
4-(5-methyl-6-(morpholin-4-yl)pyridin-3-yl)-N-(3-fluoro-4-(1,5,7-trimethyl-3,7-
diazabicyclo[3.3.1]nonan-3-yl)phenyl)pyrimidin-2-amine;
4-(6-(dimethylamino)pyridin-3-yl)-N-(3-fluoro-4-(1,5,7-trimethyl-3,7-
diazabicyclo[3.3.1]nonan-3-yl)phenyl)pyrimidin-2-amine;
4-(4-(dimethylamino)phenyl)-N-(3-fluoro-4-(1,5,7-trimethyl-3,7-diazabicyclo[3.3.1]nonan-
3-yl)phenyl)pyrimidin-2-amine;
4-(6-(morpholin-4-yl)pyridin-3-yl)-N-(3-fluoro-4-(1,5,7-trimethyl-3,7-
diazabicyclo[3.3.1]nonan-3-yl)phenyl)pyrimidin-2-amine;
4-(3,4-dihydro-2H-pyrido[3,2-b][1,4]oxazin-7-yl)-N-(3-methyl-4-((1S,4S)-5-methyl-2,5-
diazabicyclo[2.2.1]heptan-2-yl)phenyl)pyrimidin-2-amine;
4-(3,4-dihydro-2H-pyrido[3,2-b][1,4]oxazin-7-yl)-N-(3-fluoro-4-((1S,4S)-5-methyl-2,5-
diazabicyclo[2.2.1]heptan-2-yl)phenyl)pyrimidin-2-amine;
4-(3,4-dihydro-2H-pyrido[3,2-b][1,4]oxazin-7-yl)-N-(3-methyl-4-(1,5,7-trimethyl-3,7-
diazabicyclo[3.3.1]nonan-3-yl)phenyl)pyrimidin-2-amine;
4-(3,4-dihydro-2H-pyrido[3,2-b][1,4]oxazin-7-yl)-N-(3-fluoro-4-(1,5,7-trimethyl-3,7-
diazabicyclo[3.3.1]nonan-3-yl)phenyl)pyrimidin-2-amine;
4-(6-(benzyl)pyridin-3-yl)-N-(3-methyl-4-(1,5,7-trimethyl-3,7-diazabicyclo[3.3.1]nonan-3-
yl)phenyl)pyrimidin-2-amine;
4-(6-(benzyl)pyridin-3-yl)-N-(3-fluoro-4-(1,5,7-trimethyl-3,7-diazabicyclo[3.3.1]nonan-3-
yl)phenyl)pyrimidin-2-amine;
4-(2-(propyl)aminopyrimidin-5-yl)-N-(3-methyl-4-((1S,4S)-5-methyl-2,5-
diazabicyclo[2.2.1]heptan-2-yl)phenyl)pyrimidin-2-amine;
4-(2-(propyl)aminopyrimidin-5-yl)-N-(3-fluoro-4-((1S,4S)-5-methyl-2,5-
diazabicyclo[2.2.1]heptan-2-yl)phenyl)pyrimidin-2-amine;
4-(6-(cyclohexylamino)pyridin-3-yl)-N-(3-trifluoromethyl-4-((1S,4S)-5-methyl-2,5-
diazabicyclo[2.2.1]heptan-2-yl)phenyl)pyrimidin-2-amine;
4-(5-(methyl)sulfonylpyridin-3-yl)-N-(3-methyl-4-((1S,4S)-5-methyl-2,5-
diazabicyclo[2.2.1]heptan-2-yl)phenyl)pyrimidin-2-amine;
4-(6-(dimethylamino)pyridin-3-yl)-N-(3-trifluoromethyl-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-trifluoromethyl-4-((1S,4S)-5-methyl-2,5-
diazabicyclo[2.2.1]heptan-2-yl)phenyl)pyrimidin-2-amine;
4-(3,4-dihydro-2H-pyrido[3,2-b][1,4]oxazin-7-yl)-N-(3-trifluoromethyl-4-((1S,4S)-5-
methyl-2,5-diazabicyclo[2.2.1]heptan-2-yl)phenyl)pyrimidin-2-amine;
4-(6-((2-(cyclopropylsulfonyl)aminoethyl)-amino)pyridin-3-yl)-N-(3-methyl-4-((1S,4S)-5-
methyl-2,5-diazabicyclo[2.2.1]heptan-2-yl)phenyl)pyrimidin-2-amine;
4-(6-((2-(cyclopropylsulfonyl)aminoethyl)-amino)pyridin-3-yl)-N-(3-methyl-4-((1S,4S)-5-
oxa-2-azabicyclo[2.2.1]heptan-2-yl)phenyl)pyrimidin-2-amine;
4-(5-(methyl)sulfonylpyridin-3-yl)-N-(3-fluoro-4-((1S,4S)-5-methyl-2,5-
diazabicyclo[2.2.1]heptan-2-yl)phenyl)pyrimidin-2-amine;
4-(5-(methyl)sulfonylpyridin-3-yl)-N-(3-trifluoromethyl-4-((1S,4S)-5-methyl-2,5-
diazabicyclo[2.2.1]heptan-2-yl)phenyl)pyrimidin-2-amine;
4-(imidazo[1,2-a]pyridin-6-yl)-N-(3-methyl-4-((1S,4S)-5-methyl-2,5-
diazabicyclo[2.2.1]heptan-2-yl)phenyl)pyrimidin-2-amine;
4-(4-(5-(4-dimethylaminophenyl)oxazol-2-yl)phenyl)-N-(3-methyl-4-((1S,4S)-5-methyl-
2,5-diazabicyclo[2.2.1]heptan-2-yl)phenyl)pyrimidin-2-amine;
4-(6-methoxy-1H-indol-2-yl)-N-(3-methyl-4-((1S,4S)-5-methyl-2,5-
diazabicyclo[2.2.1]heptan-2-yl)phenyl)pyrimidin-2-amine;
4-(1-(3-chlorophenyl)-1H-pyrazol-4-yl)-N-(3-methyl-4-((1S,4S)-5-methyl-2,5-
diazabicyclo[2.2.1]heptan-2-yl)phenyl)pyrimidin-2-amine;
4-(1-methylbenzimidazol-6-yl)-N-(3-methyl-4-((1S,4S)-5-methyl-2,5-
diazabicyclo[2.2.1]heptan-2-yl)phenyl)pyrimidin-2-amine;
4-(5-cyano-1H-indol-2-yl)-N-(3-methyl-4-((1S,4S)-5-methyl-2,5-diazabicyclo[2.2.1]heptan-
2-yl)phenyl)pyrimidin-2-amine;
4-(1-(4-fluorophenyl)-1H-pyrazol-4-yl)-N-(3-methyl-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-methyl-4-(7-azabicyclo[2.2.1]heptan-7-
yl)phenyl)pyrimidin-2-amine;
4-(6-cyanopyridin-3-yl)-N-(3-methyl-4-((1S,4S)-5-methyl-2,5-diazabicyclo[2.2.1]heptan-2-
yl)phenyl)pyrimidin-2-amine;
4-(6-cyanopyridin-3-yl)-N-(3-methyl-4-(7-azabicyclo[2.2.1]heptan-7-yl)phenyl)pyrimidin-
2-amine;
4-(2-oxoindolin-5-yl)-N-(3-methyl-4((1S,4S)-5-methyl-2,5-diazabicyclo[2.2.1]heptan-2-
yl)phenyl)pyrimidin-2-amine;
4-(5-cyanopyridin-3-yl)-N-(3-methyl-4((1S,4S)-5-methyl-2,5-diazabicyclo[2.2.1]heptan-2-
yl)phenyl)pyrimidin-2-amine;
4-(6-(1H-tetrazol-5-yl)pyridin-3-yl)-N-(3-methyl-4((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-cyano-4-((1S,4S)-5-acetyl-2,5-
diazabicyclo[2.2.1]heptan-2-yl)phenyl)pyrimidin-2-amine;
4-(6-(morpholin-4-yl)pyridin-3-yl)-N-(3-cyano-4-((1S,4S)-5-methylsulfonyl-2,5-
diazabicyclo[2.2.1]heptan-2-yl)phenyl)pyrimidin-2-amine;
4-(6-(morpholin-4-yl)pyridin-3-yl)-N-(3-cyano-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-cyano-4-((1S,4S)-5-cyclopentyl-2,5-
diazabicyclo[2.2.1]heptan-2-yl)phenyl)pyrimidin-2-amine;
4-(6-(morpholin-4-yl)pyridin-3-yl)-N-(3-methyl-4-(1,4-diazabicyclo[3.2.1]octan-4-
yl)phenyl)pyrimidin-2-amine;
4-(2-oxoindolin-5-yl)-N-(3-methyl-4-(1,4-diazabicyclo[3.2.1]octan-4-yl)phenyl)pyrimidin-
2-amine;
(1-methylbenzimidazol-6-yl)-N-(3-methyl-4-(1,4-diazabicyclo[3.2.1]octan-4-
yl)phenyl)pyrimidin-2-amine;
4-(imidazo[1,2-a]pyridin-6-yl)-N-(3-methyl-4-(1,4-diazabicyclo[3.2.1]octan-4-
yl)phenyl)pyrimidin-2-amine;
4-(2H-benzo[b][1,4]oxazin-3(4H)-on-6-yl)-N-(3-fluoro-4-((1S,4S)-5-methyl-2,5-
diazabicyclo[2.2.1]heptan-2-yl)phenyl)pyrimidin-2-amine;
4-(2,2,4-trimethyl-3-oxo-3,4-dihydro-2H-pyrido[3,2-b][1,4]oxazin-7-yl)-N-(3-methyl-4-
((1S,4S)-5-methyl-2,5-diazabicyclo[2.2.1]heptan-2-yl)phenyl)pyrimidin-2-amine;
4-(2,2,4-trimethyl-3-oxo-3,4-dihydro-2H-pyrido[3,2-b][1,4]oxazin-7-yl)-N-(3-fluoro-4-
((1S,4S)-5-methyl-2,5-diazabicyclo[2.2.1]heptan-2-yl)phenyl)pyrimidin-2-amine;
4-(5-(3-methylpiperidin-1-yl)pyrazin-2-yl)-N-(3-fluoro-4-((1S,4S)-5-methyl-2,5-
diazabicyclo[2.2.1]heptan-2-yl)phenyl)pyrimidin-2-amine;
4-(4-(t-butylcarbonylamino)phenyl)-N-(3-fluoro-4-((1S,4S)-5-methyl-2,5-
diazabicyclo[2.2.1]heptan-2-yl)phenyl)pyrimidin-2-amine;
4-(4-(t-butylcarbonylamino)phenyl)-N-(3-methyl-4-((1S,4S)-5-methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-methyl-2,5-diazabicyclo[2.2.1]heptan-2-yl)phenyl)pyrimidin-2-amine;
4-(6-(acetamido)pyridin-3-yl)-N-(3-methyl-4-((1S,4S)-5-methyl-2,5-
diazabicyclo[2.2.1]heptan-2-yl)phenyl)pyrimidin-2-amine;
4-(4-((pyridin-2-yl)aminocarbonyl)phenyl)-N-(3-fluoro-4-((1S,4S)-5-methyl-2,5-
diazabicyclo[2.2.1]heptan-2-yl)phenyl)pyrimidin-2-amine;
4-(4-((pyridin-2-yl)aminocarbonyl)phenyl)-N-(3-methyl-4-((1S,4S)-5-methyl-2,5-
diazabicyclo[2.2.1]heptan-2-yl)phenyl)pyrimidin-2-amine;
4-(4-(methylsulfonylamino)phenyl)-N-(3-fluoro-4-((1S,4S)-5-methyl-2,5-
diazabicyclo[2.2.1]heptan-2-yl)phenyl)pyrimidin-2-amine;
4-(4-(methylsulfonylamino)phenyl)-N-(3-methyl-4-((1S,4S)-5-methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(4-((1S,4S)-5-
methyl-2,5-diazabicyclo[2.2.1]heptan-2-yl)phenyl)pyrimidin-2-amine;
4-(4-(3-cyclopropylureido)phenyl)-N-(4-((1S,4S)-5-methyl-2,5-diazabicyclo[2.2.1]heptan-
2-yl)phenyl)pyrimidin-2-amine;
4-(6-(2-(morpholin-4-yl)acetamido)pyridin-3-yl)-N-(3-methyl-4-((1S,4S)-5-methyl-2,5-
diazabicyclo[2.2.1]heptan-2-yl)phenyl)pyrimidin-2-amine;
4-(6-(2-(morpholin-4-yl)acetamido)pyridin-3-yl)-N-(4-((1S,4S)-5-methyl-2,5-
diazabicyclo[2.2.1]heptan-2-yl)phenyl)pyrimidin-2-amine;
4-(6-(2-(morpholin-4-yl)acetamido)pyridin-3-yl)-N-(3-fluoro-4-((1S,4S)-5-methyl-2,5-
diazabicyclo[2.2.1]heptan-2-yl)phenyl)pyrimidin-2-amine;
4-(6-(acetamido)pyridin-3-yl)-N-(3-methyl-4-((1S,4S)-5-oxa-2-azabicyclo[2.2.1]heptan-2-
yl)phenyl)pyrimidin-2-amine;
4-(6-aminopyridin-3-yl)-N-(3-methyl-4-((1S,4S)-5-ethylcarbonyl-2,5-
diazabicyclo[2.2.1]heptan-2-yl)phenyl)pyrimidin-2-amine;
4-(6-(acetamido)pyridin-3-yl)-N-(3-methyl-4-((1S,4S)-5-ethylcarbonyl-2,5-
diazabicyclo[2.2.1]heptan-2-yl)phenyl)pyrimidin-2-amine;
4-(6-aminopyridin-3-yl)-N-(3-methyl-4-((1S,4S)-5-methylsulfonyl-2,5-
diazabicyclo[2.2.1]heptan-2-yl)phenyl)pyrimidin-2-amine;
4-(6-(methylsulfonylamino)pyridin-3-yl)-N-(3-methyl-4-((1S,4S)-5-methyl-2,5-
diazabicyclo[2.2.1]heptan-2-yl)phenyl)pyrimidin-2-amine;
4-(6-(2-(dimethylamino)acetamido)-pyridin-3-yl)-N-(3-methyl-4-((1S,4S)-5-methyl-2,5-
diazabicyclo[2.2.1]heptan-2-yl)phenyl)pyrimidin-2-amine;
4-(6-(methylsulfonylamino)pyridin-3-yl)-N-(3-fluoro-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(4-(8-methyl-3,8-diazabicyclo[3.2.1]octan-3-
yl)phenyl)pyrimidin-2-amine;
4-(6-(methylsulfonylamino)pyridin-3-yl)-N-(4-(8-methyl-3,8-diazabicyclo[3.2.1]octan-3-
yl)phenyl)pyrimidin-2-amine;
4-(2,2-dimethyl-3-oxo-3,4-dihydro-2H-pyrido[3,2-b][1,4]oxazin-7-yl)-N-(4-(8-methyl-3,8-
diazabicyclo[3.2.1]octan-3-yl)phenyl)pyrimidin-2-amine;
4-(6-aminopyridin-3-yl)-N-(4-(8-methyl-3,8-diazabicyclo[3.2.1]octan-3-
yl)phenyl)pyrimidin-2-amine;
4-(6-(morpholin-4-yl)pyridin-3-yl)-N-(3-methyl-4-(1,4-diazabicyclo[3.2.2]nonan-4-
yl)phenyl)pyrimidin-2-amine;
4-(2,2-dimethyl-3-oxo-3,4-dihydro-2H-pyrido[3,2-b][1,4]oxazin-7-yl)-N-(3-methyl-4-(1,4-
diazabicyclo[3.2.2]nonan-4-yl)phenyl)pyrimidin-2-amine;
4-(4-((pyridin-2-yl)aminocarbonyl)phenyl)-N-(3-methyl-4-(1,4-diazabicyclo[3.2.2]nonan-4-
yl)phenyl)pyrimidin-2-amine;
4-(4-(acetamido)phenyl)-N-(4-(8-methyl-3,8-diazabicyclo[3.2.1]octan-3-
yl)phenyl)pyrimidin-2-amine;
4-(2-(diethylamino)thiazol-4-yl)-N-(3-methyl-4-((1S,4S)-5-methyl-2,5-
diazabicyclo[2.2.1]heptan-2-yl)phenyl)pyrimidin-2-amine;
4-(2-(diethylamino)thiazol-4-yl)-N-(3-fluoro-4-((1S,4S)-5-methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-(8-
methyl-3,8-diazabicyclo[3.2.1]octan-3-yl)phenyl)pyrimidin-2-amine;
4-(6-(morpholin-4-yl)pyridin-3-yl)-N-(3-methyl-4-(8-methyl-3,8-diazabicyclo[3.2.1]octan-
3-yl)phenyl)pyrimidin-2-amine;
4-(6-(methylsulfonylamino)pyridin-3-yl)-N-(3-methyl-4-(8-methyl-3,8-
diazabicyclo[3.2.1]octan-3-yl)phenyl)pyrimidin-2-amine;
4-(4-(t-butylcarbonylamino)phenyl)-N-(3-methyl-4-(8-methyl-3,8-diazabicyclo[3.2.1]octan-
3-yl)phenyl)pyrimidin-2-amine;
4-(1H-pyrrol-3-yl)-N-(3-methyl-4-((1S,4S)-5-methyl-2,5-diazabicyclo[2.2.1]heptan-2-
yl)phenyl)pyrimidin-2-amine;
4-(1H-pyrrol-3-yl)-N-(4-((1S,4S)-5-methyl-2,5-diazabicyclo[2.2.1]heptan-2-
yl)phenyl)pyrimidin-2-amine;
4-(1H-pyrrol-3-yl)-N-(3-trifluoromethyl-4-((1S,4S)-5-methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-
trifluoromethyl-4-((1S,4S)-2,5-diazabicyclo[2.2.1]heptan-2-yl)phenyl)pyrimidin-2-amine;
4-(6-ethoxypyridin-3-yl)-N-(3-methyl-4-((1S,4S)-5-methyl-2,5-diazabicyclo[2.2.1]heptan-
2-yl)phenyl)pyrimidin-2-amine;
4-(6-ethoxypyridin-3-yl)-N-(3-fluoro-4-((1S,4S)-5-methyl-2,5-diazabicyclo[2.2.1]heptan-2-
yl)phenyl)pyrimidin-2-amine;
4-(2-(dimethylamino)thiazol-4-yl)-N-(3-methyl-4-((1S,4S)-5-methyl-2,5-
diazabicyclo[2.2.1]heptan-2-yl)phenyl)pyrimidin-2-amine;
4-(2-(dimethylamino)thiazol-4-yl)-N-(3-fluoro-4-((1S,4S)-5-methyl-2,5-
diazabicyclo[2.2.1]heptan-2-yl)phenyl)pyrimidin-2-amine;
4-(5-(morpholin-4-yl)pyrazin-2-yl)-N-(3-methyl-4-((1S,4S)-5-methyl-2,5-
diazabicyclo[2.2.1]heptan-2-yl)phenyl)pyrimidin-2-amine;
4-(5-(morpholin-4-yl)pyrazin-2-yl)-N-(3-fluoro-4-((1S,4S)-5-methyl-2,5-
diazabicyclo[2.2.1]heptan-2-yl)phenyl)pyrimidin-2-amine;
4-(4-(1-ethoxyethyl)phenyl)-N-(3-methyl-4-((1S,4S)-5-methyl-2,5-
diazabicyclo[2.2.1]heptan-2-yl)phenyl)pyrimidin-2-amine;
4-(4-(1-ethoxyethyl)phenyl)-N-(3-fluoro-4-((1S,4S)-5-methyl-2,5-
diazabicyclo[2.2.1]heptan-2-yl)phenyl)pyrimidin-2-amine;
4-(6-(dimethylamino)pyridin-3-yl)-N-(4-((1S,4S)-5-methyl-2,5-diazabicyclo[2.2.1]heptan-
2-yl)phenyl)pyrimidin-2-amine;
4-(6-(morpholin-4-yl)pyridin-3-yl)-N-(4-((1S,4S)-5-methyl-2,5-diazabicyclo[2.2.1]heptan-
2-yl)phenyl)pyrimidin-2-amine;
4-(4-methyl-3,4-dihydro-2H-pyrido[3,2-b][1,4]oxazin-7-yl)-N-(4-((1S,4S)-5-methyl-2,5-
diazabicyclo[2.2.1]heptan-2-yl)phenyl)pyrimidin-2-amine;
4-(6-(3-ethoxypropyl)aminopyridin-3-yl)-N-(3-methyl-4-((1S,4S)-5-methyl-2,5-
diazabicyclo[2.2.1]heptan-2-yl)phenyl)pyrimidin-2-amine;
4-(6-(cis-2,6-dimethylmorpholin-4-yl)pyridin-3-yl)-N-(3-methyl-4-((1S,4S)-5-methyl-2,5;
diazabicyclo[2.2.1]heptan-2-yl)phenyl)pyrimidin-2-amine;
4-(6-(propylamino)pyridin-3-yl)-N-(3-methyl-4-((1S,4S)-5-methyl-2,5-
diazabicyclo[2.2.1]heptan-2-yl)phenyl)pyrimidin-2-amine;
4-(6-(2-(dimethylamino)methylmorpholin-4-yl)pyridin-3-yl)-N-(3-methyl-4-((1S,4S)-5-
methyl-2,5-diazabicyclo[2.2.1]heptan-2-yl)phenyl)pyrimidin-2-amine;
4-(6-(piperidin-1-yl)pyridin-3-yl)-N-(3-methyl-4-((1S,4S)-5-methyl-2,5-
diazabicyclo[2.2.1]heptan-2-yl)phenyl)pyrimidin-2-amine;
4-(6-(3-(aminocarbonyl)piperidin-1-yl)pyridin-3-yl)-N-(3-methyl-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-methyl-4-((1S,4S)-5-methylsulfonyl-2,5-
diazabicyclo[2.2.1]heptan-2-yl)phenyl)pyrimidin-2-amine;
4-(4-methyl-3,4-dihydro-2H-pyrido[3,2-b][1,4]oxazin-7-yl)-N-(3-methyl-4-((1S,4S)-5-
methylsulfonyl-2,5-diazabicyclo[2.2.1]heptan-2-yl)phenyl)pyrimidin-2-amine;
4-(6-(morpholin-4-yl)pyridin-3-yl)-N-(3-methyl-4-((1S,4S)-5-oxa-2-
azabicyclo[2.2.1]heptan-2-yl)phenyl)pyrimidin-2-amine;
4-(4-methyl-3,4-dihydro-2H-pyrido[3,2-b][1,4]oxazin-7-yl)-N-(3-methyl-4-((1S,4S)-5-oxa-
2-azabicyclo[2.2.1]heptan-2-yl)phenyl)pyrimidin-2-amine;
4-(6-(morpholin-4-yl)pyridin-3-yl)-N-(3-methyl-4-((1S,4S)-5-ethylcarbonyl-2,5-
diazabicyclo[2.2.1]heptan-2-yl)phenyl)pyrimidin-2-amine;
4-(6-(2-(morpholin-4-yl)ethyl)aminopyridin-3-yl)-N-(3-methyl-4-((1S,4S)-5-ethylcarbonyl-
2,5-diazabicyclo[2.2.1]heptan-2-yl)phenyl)pyrimidin-2-amine;
4-(6-(3-dimethylamino)propylaminopyridin-3-yl)-N-(3-methyl-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-methyl-4-((1S,4S)-5-amidino-2,5-
diazabicyclo[2.2.1]heptan-2-yl)phenyl)pyrimidin-2-amine TFA salt;
4-(6-(3-ethoxypropyl)aminopyridin-3-yl)-N-(3-methyl-4-((1S,4S)-5-oxa-2-
azabicyclo[2.2.1]heptan-2-yl)phenyl)pyrimidin-2-amine;
4-(6-cis-2,6-dimethylmorpholin-4-yl)pyridin-3-yl)-N-(3-methyl-4-((1S,4S)-5-oxa-2-
azabicyclo[2.2.1]heptan-2-yl)phenyl)pyrimidin-2-amine;
4-(6-(propylamino)pyridin-3-yl)-N-(3-methyl-4-((1S,4S)-5-oxa-2-azabicyclo[2.2.1]heptan-
2-yl)phenyl)pyrimidin-2-amine;
4-(6-(3-dimethylamino)propylaminopyridin-3-yl)-N-(3-methyl-4-((1S,4S)-5-oxa-2-
azabicyclo[2.2.1]heptan-2-yl)phenyl)pyrimidin-2-amine;
4-(6-(1,4-oxazepan-4-yl)pyridin-3-yl)-N-(3-methyl-4-((1S,4S)-5-oxa-2-
azabicyclo[2.2.1]heptan-2-yl)phenyl)pyrimidin-2-amine;
4-(6-(morpholin-4-yl)pyridin-3-yl)-N-(3-methyl-4-((1S,4S)-5-isobutyl-2,5-
diazabicyclo[2.2.1]heptan-2-yl)phenyl)pyrimidin-2-amine;
4-(6-(1,4-oxazepan-4-yl)pyridin-3-yl)-N-(3-methyl-4-((1S,4S)-5-methyl-2,5-
diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
(1,5,7-trimethyl-3,7-diazabicyclo[3.3.1]nonan-3-yl)phenyl)pyrimidin-2-amine;
4-(2,2-dimethyl-3-oxo-3,4-dihydro-2H-pyrido[3,2-b][1,4]oxazin-7-yl)-N-(3-fluoro-4-(1,5,7-
trimethyl-3,7-diazabicyclo[3.3.1]nonan-3-yl)phenyl)pyrimidin-2-amine;
4-(3-oxo-3,4-dihydro-2H-pyrido[3,2-b][1,4]oxazin-7-yl)-N-(3-methyl-4-((1S,4S)-5-methyl-
2,5-diazabicyclo[2.2.1]heptan-2-yl)phenyl)pyrimidin-2-amine;
4-(3-oxo-3,4-dihydro-2H-pyrido[3,2-b][1,4]oxazin-7-yl)-N-(3-methyl-4-((1S,4S)-5-oxa-2-
azabicyclo[2.2.1]heptan-2-yl)phenyl)pyrimidin-2-amine;
4-(6-(3-ethoxypropyl)aminopyridin-3-yl)-N-(3-methyl-4-(1,5,7-trimethyl-3,7-
diazabicyclo[3.3.1]nonan-3-yl)phenyl)pyrimidin-2-amine;
4-(6-((2S,6R)-2,6-dimethylmorpholin-4-yl)pyridin-3-yl)-N-(3-methyl-4-(1,5,7-trimethyl-
3,7-diazabicyclo[3.3.1]nonan-3-yl)phenyl)pyrimidin-2-amine;
4-(6-(propylamino)pyridin-3-yl)-N-(3-methyl-4-(1,5,7-trimethyl-3,7-
diazabicyclo[3.3.1]nonan-3-yl)phenyl)pyrimidin-2-amine;
4-(6-(3-dimethylamino)propylaminopyridin-3-yl)-N-(3-methyl-4-(1,5,7-trimethyl-3,7-
diazabicyclo[3.3.1]nonan-3-yl)phenyl)pyrimidin-2-amine;
4-(6-((1S,4S)-2-oxa-5-azabicyclo[2.2.1]heptan-5-yl)pyridin-3-yl)-N-(3-methyl-4-((1S,4S)-5-
methyl-2,5-diazabicyclo[2.2.1]heptan-2-yl)phenyl)pyrimidin-2-amine;
4-(6-((1S,4S)-5-oxa-2-azabicyclo[2.2.1]heptan-2-yl)pyridin-3-yl)-N-(3-methyl-4-((1S,4S)-5-
oxa-2-azabicyclo[2.2.1]heptan-2-yl)phenyl)pyrimidin-2-amine;
4-(6-(3-methylbutyl)aminopyridin-3-yl)-N-(3-methyl-4-((1S,4S)-5-methyl-2,5-
diazabicyclo[2.2.1]heptan-2-yl)phenyl)pyrimidin-2-amine;
4-(6-(3,3-dimethylbutyl)aminopyridin-3-yl)-N-(3-methyl-4-((1S,4S)-5-methyl-2,5-
diazabicyclo[2.2.1]heptan-2-yl)phenyl)pyrimidin-2-amine;
4-(6-(2-methoxyethyl)(methyl)aminopyridin-3-yl)-N-(3-methyl-4-((1S,4S)-5-methyl-2,5-
diazabicyclo[2.2.1]heptan-2-yl)phenyl)pyrimidin-2-amine;
4-(6-(2-methoxyethyl)(methyl)aminopyridin-3-yl)-N-(3-methyl-4-((1S,4S)-5-oxa-2-
azabicyclo[2.2.1]heptan-2-yl)phenyl)pyrimidin-2-amine;
4-(6-(2-methoxyethyl)(methyl)aminopyridin-3-yl)-N-(3-methyl-4-(1,5,7-trimethyl-3,7-
diazabicyclo[3.3.1]nonan-3-yl)phenyl)pyrimidin-2-amine;
4-(6-(morpholin-4-yl)pyridin-3-yl)-N-(3-methyl-4-((1S,4R)-2-azabicyclo[2.2.1]heptan-2-
yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4R)-2-azabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-(2-
methylsulfonyl-2-azabicyclo[2.2.1]heptan-5-yl)phenyl)pyrimidin-2-amine and 4-(2,2-dimethyl-
3-oxo-3,4-dihydro-2H-pyrido[3,2-b][1,4]oxazin-7-yl)-N-(3-methyl-4-(2-methylsulfonyl-2-
azabicyclo[2.2.1]heptan-6-yl)phenyl)pyrimidin-2-amine;
4-(6-(morpholin-4-yl)pyridin-3-yl)-N-(3-methyl-4-(2-methylsulfonyl-2-
azabicyclo[2.2.1]heptan-5-yl)phenyl)pyrimidin-2-amine and 4-(6-(morpholin-4-yl)pyridin-3-
yl)-N-(3-methyl-4-(2-methylsulfonyl-2-azabicyclo[2.2.1]heptan-6-yl)phenyl)pyrimidin-2-
amine;
4-(2,2-dimethyl-3-oxo-3,4-dihydro-2H-pyrido[3,2-b][1,4]oxazin-7-yl)-N-(4-(2-
methylsulfonyl-2-azabicyclo[2.2.1]heptan-5-yl)phenyl)pyrimidin-2-amine;
4-(6-(morpholin-4-yl)pyridin-3-yl)-N-(4-(2-methylsulfonyl-2-azabicyclo[2.2.1]heptan-5-
yl)phenyl)pyrimidin-2-amine;
4-(2,2-dimethyl-3-oxo-3,4-dihydro-2H-pyrido[3,2-b][1,4]oxazin-7-yl)-N-(4-(2-
methylsulfonyl-2-azabicyclo[2.2.1]heptan-6-yl)phenyl)pyrimidin-2-amine;
4-(6-(morpholin-4-yl)pyridin-3-yl)-N-(4-(2-methylsulfonyl-2-azabicyclo[2.2.1]heptan-6-
yl)phenyl)pyrimidin-2-amine;
4-(6-(thiamorpholin-4-yl)pyridin-3-yl)-N-(3-methyl-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-methyl-4-((1S,4S)-5-methyl-2,5-
diazabicyclo[2.2.1]heptan-2-yl)phenyl)pyrimidin-2-amine;
4-(1-(pyridin-4-yl)-1H-indol-5-yl)-N-(3-methyl-4-((1S,4S)-5-methyl-2,5-
diazabicyclo[2.2.1]heptan-2-yl)phenyl)pyrimidin-2-amine;
4-(1-(pyridin-4-yl)-1H-indol-5-yl)-N-(3-fluoro-4-((1S,4S)-5-methyl-2,5-
diazabicyclo[2.2.1]heptan-2-yl)phenyl)pyrimidin-2-amine;
4-(1-(pyridin-4-yl)-1H-indol-5-yl)-N-(3-methyl-4-((1S,4S)-5-ethyl-2,5-
diazabicyclo[2.2.1]heptan-2-yl)phenyl)pyrimidin-2-amine;
4-(1-(pyridin-4-yl)-1H-indol-5-yl)-N-(3-methyl-4-((1S,4S)-5-isobutyl-2,5-
diazabicyclo[2.2.1]heptan-2-yl)phenyl)pyrimidin-2-amine;
4-(7-oxo-5,6,7,8-tetrahydro-1,8-naphthyridin-3-yl)-N-(3-methyl-4-((1S,4S)-5-
methylsulfonyl-2,5-diazabicyclo[2.2.1]heptan-2-yl)phenyl)pyrimidin-2-amine;
4-(6-(propylamino)pyridin-3-yl)-N-(3-methyl-4-((1S,4S)-5-methylsulfonyl-2,5-
diazabicyclo[2.2.1]heptan-2-yl)phenyl)pyrimidin-2-amine;
4-(3-oxo-3,4-dihydro-2H-benzo[b][1,4]thiazin-7-yl)-N-(4-((1S,4S)-5-methyl-2,5-
diazabicyclo[2.2.1]heptan-2-yl)phenyl)pyrimidin-2-amine;
4-(2,2-dimethyl-3-oxo-3,4-dihydro-2H-benzo[b][1,4]oxazin-7-yl)-N-(3-methyl-4-((1S,4S)-
5-methyl-2,5-diazabicyclo[2.2.1]heptan-2-yl)phenyl)pyrimidin-2-amine;
4-(2,2-dimethyl-3-oxo-3,4-dihydro-2H-benzo[b][1,4]oxazin-7-yl)-N-(3-methyl-4-((1S,4S)-
5-methylsulfonyl-2,5-diazabicyclo[2.2.1]heptan-2-yl)phenyl)pyrimidin-2-amine;
4-(2-oxo-2,3,4,5-tetrahydro-1H-benzo[b]azepin-7-yl)-N-(3-methyl-4-((1S,4S)-5-methyl-2,5-
diazabicyclo[2.2.1]heptan-2-yl)phenyl)pyrimidin-2-amine;
4-(2-oxo-2,3,4,5-tetrahydro-1H-benzo[b]azepin-7-yl)-N-(3-methyl-4-((1S,4S)-5-
methylsulfonyl-2,5-diazabicyclo[2.2.1]heptan-2-yl)phenyl)pyrimidin-2-amine;
4-(2,2-dimethyl-3-oxo-3,4-dihydro-2H-benzo[b][1,4]oxazin-7-yl)-N-(3-methyl-4-((6R,9S)-
6,9-methanooctahydro-1H-pyrido[1,2-a]pyrazin-2-yl)phenyl)pyrimidin-2-amine;
4-(6-(morpholin-4-yl)pyridin-3-yl)-N-(3-methyl-4-((6R,9S)-6,9-methanooctahydro-1H-
pyrido[1,2-a]pyrazin-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-(1-methylethyl)-2,5-diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-cyclopropyl-2,5-diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-chloro-4-
((1S,4S)-5-methyl-2,5-diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-chloro-4-
((1S,4S)-5-(methylsulfonyl)-2,5-diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-cyclopentyl-2,5-diazabicydo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-
((1S,4S)-5-acetyl-2,5-diazabicyclo[2.2.1]heptan-2-yl)phenyl)pyrimidin-2-amine;
4-(2,2-dimethyl-3-oxo-3,4-dihydro-2H-pyrido[3,2-b][1,4]oxazin-7-yl)-N-(3-methyl-4-(1,4-
diazabicyclo[3.2.1]octan-4-yl)phenyl)pyrimidin-2-amine;
4-(5-(1-methylethoxy)carbonylpropyl-6-aminopyridin-3-yl)-N-(3-methyl-4-((1S,4S)-5-
methyl-2,5-diazabicyclo[2.2.1]heptan-2-yl)phenyl)pyrimidin-2-amine;
4-(6-(morpholin-4-yl)pyridin-3-yl)-N-(3-methyl-4-((1S,4S)-5-cyclopropyl-2,5-
diazabicyclo[2.2.1]heptan-2-yl)phenyl)pyrimidin-2-amine;
4-(6-(morpholin-4-yl)pyridin-3-yl)-N-(3-chloro-4-((1S,4S)-5-methyl-2,5-
diazabicyclo[2.2.1]heptan-2-yl)phenyl)pyrimidin-2-amine;
4-(2-(trifluoromethyl)pyridin-4-yl)-N-(3-methyl-4-((1S,4S)-5-methyl-2,5-
diazabicyclo[2.2.1]heptan-2-yl)phenyl)pyrimidin-2-amine;
4-(6-(morpholin-4-yl)pyridin-3-yl)-N-(3-chloro-4-((1S,4S)-5-(methylsulfonyl)-2,5-
diazabicyclo[2.2.1]heptan-2-yl)phenyl)pyrimidin-2-amine;
4-(6-(tetrahydropyran-4-yloxy)pyridin-3-yl)-N-(3-chloro-4-((1S,4S)-5-(methylsulfonyl)-2,5-
diazabicyclo[2.2.1]heptan-2-yl)phenyl)pyrimidin-2-amine;
4-(6-(morpholin-4-yl)pyridin-3-yl)-N-(3-chloro-4-((1S,4S)-5-acetyl-2,5-
diazabicyclo[2.2.1]heptan-2-yl)phenyl)pyrimidin-2-amine;
4-(6-(tetrahydropyran-4-yloxy)pyridin-3-yl)-N-(3-chloro-4-((1S,4S)-5-acetyl-2,5-
diazabicyclo[2.2.1]heptan-2-yl)phenyl)pyrimidin-2-amine;
4-(6-(tetrahydropyran-4-yloxy)pyridin-3-yl)-N-(3-methyl-4-((1S,4S)-5-methyl-2,5-
diazabicyclo[2.2.1]heptan-2-yl)phenyl)pyrimidin-2-amine;
4-(6-(tetrahydropyran-4-yloxy)pyridin-3-yl)-N-(3-methyl-4-((1S,4S)-5-cyclopentyl-2,5-
diazabicyclo[2.2.1]heptan-2-yl)phenyl)pyrimidin-2-amine;
4-(6-(morpholin-4-yl)pyridin-3-yl)-N-(3-chloro-4-((1S,4S)-5-(1-methylethyl)-2,5-
diazabicyclo[2.2.1]heptan-2-yl)phenyl)pyrimidin-2-amine;
4-(6-((1S,4S)-2-oxa-5-azabicyclo[2.2.1]heptan-5-yl)pyridin-3-yl)-N-(3-methyl-4-((1S,4S)-5-
(methylsulfonyl)-2,5-diazabicyclo[2.2.1]heptan-2-yl)phenyl)pyrimidin-2-amine;
4-(6-((1R,4R)-2-oxa-5-azabicyclo[2.2.1]heptan-5-yl)pyridin-3-yl)-N-(3-methyl-4-((1S,4S)-
5-methyl-2,5-diazabicyclo[2.2.1]heptan-2-yl)phenyl)pyrimidin-2-amine;
4-(6,7,8,9-tetrahydro-5H-pyrido[2,3-b]indol-3-yl)-N-(3-methyl-4-((1S,4S)-5-methyl-2,5-
diazabicyclo[2.2.1]heptan-2-yl)phenyl)pyrimidin-2-amine;
4-(6,7,8,9-tetrahydro-5H-pyrido[2,3-b]indol-3-yl)-N-(3-methyl-4-((1S,4S)-5-
(methylsulfonyl)-2,5-diazabicyclo[2.2.1]heptan-2-yl)phenyl)pyrimidin-2-amine;
4-(4-(trifluoromethyl)phenyl)-N-(3-methyl-4-((1S,4S)-5-methyl-2,5-
diazabicyclo[2.2.1]heptan-2-yl)phenyl)pyrimidin-2-amine;
4-(7,8,9,9a-tetrahydro-5H-pyrido[2,3-e]pyrrolo[1,2-a][1,4]diazepin-10(11H)-on-3-yl)-N-(3-
methyl-4-((1S,4S)-5-methyl-2,5-diazabicyclo[2.2.1]heptan-2-yl)phenyl)pyrimidin-2-amine;
4-(2,2-dimethyl-3-oxo-3,4-dihydro-2H-benzo[b][1,4]oxazin-7-yl)-N-(3-cyano-4-((1S,4S)-5-
methyl-2,5-diazabicyclo[2.2.1]heptan-2-yl)phenyl)pyrimidin-2-amine;
4-(2-oxo-2,3,4,5-tetrahydro-1H-benzo[b]azepin-7-yl)-N-(3-cyano-4-((1S,4S)-5-methyl-2,5-
diazabicyclo[2.2.1]heptan-2-yl)phenyl)pyrimidin-2-amine;
4-(3-oxo-3,4-dihydro-2H-benzo[b][1,4]thiazin-7-yl)-N-(3-cyano-4-((1S,4S)-5-methyl-2,5-
diazabicyclo[2.2.1]heptan-2-yl)phenyl)pyrimidin-2-amine;
4-(3-oxo-3,4-dihydro-2H-benzo[b][1,4]thiazin-7-yl)-N-(3-methyl-4-((1S,4S)-5-methyl-2,5-
diazabicyclo[2.2.1]heptan-2-yl)phenyl)pyrimidin-2-amine;
4-(6-(tetrahydropyran-4-yloxy)pyridin-3-yl)-N-(3-methyl-4-(1,4-diazabicyclo[3.2.1]octan-
4-yl)phenyl)pyrimidin-2-amine;
4-(6-(morpholin-4-yl)pyridin-3-yl)-N-(3-methyl-4-((R)-1,4-diazabicyclo[3.2.1]octan-4-
yl)phenyl)pyrimidin-2-amine;
4-(6-(morpholin-4-yl)pyridin-3-yl)-N-(3-methyl-4-((S)-1,4-diazabicyclo[3.2.1]octan-4-
yl)phenyl)pyrimidin-2-amine;
4-(2-oxo-2,3,4,5-tetrahydro-1H-benzo[b]azepin-7-yl)-N-(3-methyl-4-((6R,9S)-6,9-
methanooctahydro-1H-pyrido[1,2-a]pyrazin-2-yl)phenyl)pyrimidin-2-amine;
4-(3-oxo-3,4-dihydro-2H-benzo[b][1,4]thiazin-7-yl)-N-(3-methyl-4-((6R,9S)-6,9-
methanooctahydro-1H-pyrido[1,2-a]pyrazin-2-yl)phenyl)pyrimidin-2-amine;
4-(6-((1S,4S)-2-oxa-5-azabicyclo[2.2.1]heptan-5-yl)pyridin-3-yl)-N-(3-methyl-4-(1,4-
diazabicyclo[3.2.2]nonan-4-yl)phenyl)pyrimidin-2-amine;
4-(6-(morpholin-4-yl)pyridin-3-yl)-N-(3-cyano-4-(3,9-diazabicyclo[3.3.2]decan-10-on-3-
yl)phenyl)pyrimidin-2-amine;
4-(2,2-dimethyl-3-oxo-3,4-dihydro-2H-pyrido[3,2-b][1,4]oxazin-7-yl)-N-(3-cyano-4-(3,9-
diazabicyclo[3.3.2]decan-10-on-3-yl)phenyl)pyrimidin-2-amine;
4-(6-(tetrahydropyran-4-yloxy)pyridin-3-yl)-N-(3-methyl-4-(3-(dimethylamino)-8-
azabicyclo[3.2.1]octan-8-yl)phenyl)pyrimidin-2-amine;
4-(6-(morpholin-4-yl)pyridin-3-yl)-N-(3-methyl-4-(3-(morpholin-4-yl)-8-
azabicyclo[3.2.1]octan-8-yl)phenyl)pyrimidin-2-amine;
4-(6-(tetrahydropyran-4-yloxy)pyridin-3-yl)-N-(3-methyl-4-(3-(morpholin-4-yl)-8-
azabicyclo[3.2.1]octan-8-yl)phenyl)pyrimidin-2-amine;
4-(6-(morpholin-4-yl)pyridin-3-yl)-N-(3-methyl-4-(3-(dimethylamino)-8-
azabicyclo[3.2.1]octan-8-yl)phenyl)pyrimidin-2-amine;
N4-(4-chloro-3-methoxyphenyl)-5-methyl-N2-(4-(2-(pyrrolidin-1-
yl)ethoxy)phenyl)pyrimidine-2,4-diamine; or
N-(tert-butyl)-3-((5-methyl-2-((4-(2-(pyrrolidin-1-yl)ethoxy)phenyl)amino)pyrimidin-4-
yl)amino)benzenesulfonamide.

In other disclosed embodiments, the one or more compounds may be a compound selected from the compounds provided below in Table II.

TABLE II
3-{(3R,4R)-4-methyl-3-[methyl-(7H-pyrrolo[2,3-d]pyrimidin-4-yl)-amino]-piperidin-1-
yl}-3-oxo-propionitrile(tofacitinib) and the citrate salt thereof;
(R)-3-(4-(7H-pyrrolo[2,3-d]pyrimidin-4-yl)-1H-pyrazol-1-yl)-3-cyclopentyl-
propanenitrile(ruxolitinib);
3-[3-methyl-1-(1H-pyrrolo[2,3-b]pyridin-4-yl)-1H-pyrazol-4-yl]benzonitrile;
3-[3-methyl-1-(1H-pyrrolo[2,3-b]pyridin-4-yl)-1H-pyrazol-4-yl]acrylonitrile;
3-[3-methyl-1-(1H-pyrrolo[2,3-b]pyridin-4-yl)-1H-pyrazol-4-yl]propanenitrile;
4-(4-phenyl-1H-imidazol-1-yl)-1H-pyrrolo[2,3-b]pyridine;
[3-methyl-1-(1H-pyrrolo[2,3-b]pyridin-4-yl)-1H-pyrazol-4-yl]-piperidin-1-yl-methanone;
[3-methyl-1-(1H-pyrrolo[2,3-b]pyridin-4-yl)-1H-pyrazol-4-ylmethyl]-phenyl-amine;
3-[3-methyl-1-(1H-pyrrolo[2,3-b]pyridin-4-yl)-1H-pyrazol-4-yl]-cyclohexanol;
4-[1-(3-methoxy-1-methyl-propyl)-1H-pyrazol-4-yl]-1H-pyrrolo[2,3-b]pyridine;
4-[1-(1-methyl-3-pyrazol-1-yl-propyl)-1H-pyrazol-4-yl]-1H-pyrrolo[2,3-b]pyridine;
1-(1H-pyrrolo[2,3-b]pyridin-4-yl)-1H-pyrazole-4-carboxylic acid ethyl ester;
4-(3-methyl-4-phenyl-pyrazol-1-yl)-1H-pyrrolo[2,3-b]pyridine;
4-(3-phenyl-pyrazol-1-yl)-1H-pyrrolo[2,3-b]pyridine;
4-(4-bromo-imidazol-1-yl)-1H-pyrrolo[2,3-b]pyridine;
4-(4-bromo-3-methyl-pyrazol-1-yl)-1H-pyrrolo[2,3-b]pyridine;
3-[3-methyl-1-(1H-pyrrolo[2,3-b]pyridin-4-yl)-1H-pyrazol-4-yl]-benzonitrile;
4-[3-methyl-1-(1H-pyrrolo[2,3-b]pyridin-4-yl)-1H-pyrazol-4-yl]-benzonitrile;
4-[4-(3-fluoro-phenyl)-3-methyl-pyrazol-1-yl]-1H-pyrrolo[2,3-b]pyridine;
4-[4-(3,5-bis-trifluoromethyl-phenyl)-3-methyl-pyrazol-1-yl]-1H-pyrrolo[2,3-b]pyridine;
4-[4-(3,5-difluoro-phenyl)-3-methyl-pyrazol-1-yl]-1H-pyrrolo[2,3-b]pyridine;
{3-[3-methyl-1-(1H-pyrrolo[2,3-b]pyridin-4-yl)-1H-pyrazol-4-yl]-phenyl}-methanol;
4-(3-methyl-4-pyrimidin-5-yl-pyrazol-1-yl)-1H-pyrrolo[2,3-b]pyridine;
4-[3-methyl-4-(1-methyl-1H-indol-5-yl)-pyrazol-1-yl]-1H-pyrrolo[2,3-b]pyridine;
4-(3-methyl-4-thiophen-3-yl-pyrazol-1-yl)-1H-pyrrolo[2,3-b]pyridine;
N,N-dimethyl-4-[3-methyl-1-(1H-pyrrolo[2,3-b]pyridin-4-yl)-1H-pyrazol-4-yl]-
benzene-sulfonamide;
N-{4-[3-methyl-1-(1H-pyrrolo[2,3-b]pyridin-4-yl)-1H-pyrazol-4-yl]-phenyl}-acetamide;
3-tert-butyl-1-(1H-pyrrolo[2,3-b]pyridin-4-yl)-1H-pyrazole-4-carbonitrile;
4-bromo-1-(1H-pyrrolo[2,3-b]pyridin-4-yl)-1H-pyrazole-3-carbonitrile;
4-(3-cyano-phenyl)-1-(1H-pyrrolo[2,3-b]pyridin-4-yl)-1H-pyrazole-3-carbonitrile;
3-[1-(1H-pyrrolo[2,3-b]pyridin-4-yl)-3-trifluoromethyl-1H-pyrazol-4-yl]-propan-1-ol;
3-[3-methyl-1-(1H-pyrrolo[2,3-b]pyridin-4-yl)-1H-pyrazol-4-yl]-prop-2-en-1-ol;
2-[4-bromo-1-(1H-pyrrolo[2,3-b]pyridin-4-yl)-1H-pyrazol-3-yl]-isoindole-1,3-dione;
4-[4-(2,6-dimethyl-phenyl)-3-methyl-pyrazol-1-yl]-1H-pyrrolo[2,3-b]pyridine;
3-[3-amino-1-(1H-pyrrolo[2,3-b]pyridin-4-yl)-1H-pyrazol-4-yl]-benzonitrile;
3-[3-benzylamino-1-(1H-pyrrolo[2,3-b]pyridin-4-yl)-1H-pyrazol-4-yl]-benzonitrile;
N-[4-(3-cyano-phenyl)-1-(1H-pyrrolo[2,3-b]pyridin-4-yl)-1H-pyrazol-3-yl]-acetamide;
3-[4-(1H-pyrrolo[2,3-b]pyridin-4-yl)-pyrazol-1-yl]-propan-1-ol;
3-[4-(1H-pyrrolo[2,3-b]pyridin-4-yl)-pyrazol-1-yl]-butan-1-ol;
4-[4-(1H-pyrrolo[2,3-b]pyridin-4-yl)-pyrazol-1-yl]-pentanenitrile;
4-[4-(1H-pyrrolo[2,3-b]pyridin-4-yl)-pyrazol-1-yl]-pentanoic acid amide;
4-[1-(3-imidazol-1-yl-1-methyl-propyl)-1H-pyrazol-4-yl]-1H-pyrrolo[2,3-b]pyridine;
4-cyclopentyl-4-[4-(1H-pyrrolo[2,3-b]pyridin-4-yl)-pyrazol-1-yl]-butyronitrile;
4-cyclopentyl-4-[4-(1H-pyrrolo[2,3-b]pyridin-4-yl)-pyrazol-1-yl]-butyramide;
3-cyclopropyl-3-[4-(7H-pyrrolo[2,3-d]pyrimidin-4-yl)-pyrazol-1-yl]-propionitrile;
4-(2-tert-butyl-1-methyl-1H-imidazol-4-yl)-1H-pyrrolo[2,3-b]pyridine;
4-(2-phenyl-1H-imidazol-5-yl)-1H-pyrrolo[2,3-b]pyridine;
4-(2-benzyl-1H-imidazol-5-yl)-1H-pyrrolo[2,3-b]pyridine;
4-[2-(1-phenylethyl)-1H-imidazol-5-yl]-1H-pyrrolo[2,3-b]pyridine;
4-(2-phenyl-1,3-thiazol-4-yl)-1H-pyrrolo[2,3-b]pyridine;
N-methyl-N-propyl-4-(1H-pyrrolo[2,3-b]pyridin-4-yl)-1,3-thiazol-2-amine;
N-phenyl-4-(1H-pyrrolo[2,3-b]pyridin-4-yl)-1,3-thiazol-2-amine;
N-methyl-N-phenyl-4-(1H-pyrrolo[2,3-b]pyridin-4-yl)-1,3-thiazol-2-amine;
4-(2-phenyl-1,3-thiazol-5-yl)-1H-pyrrolo[2,3-b]pyridine;
ethyl2-methyl-2-[4-(1H-pyrrolo[2,3-b]pyridin-4-yl)-1H-pyrazol-1-yl]propanoate;
2-methyl-2-[4-(1H-pyrrolo[2,3-b]pyridin-4-yl)-1H-pyrazol-1-yl]propanoic acid;
2-methyl-2-[4-(1H-pyrrolo[2,3-b]pyridin-4-yl)-1H-pyrazol-1-yl]propanamide;
ethyl3-methyl-3-[4-(1H-pyrrolo[2,3-b]pyridin-4-yl)-1H-pyrazol-1-yl]butanoate;
3-methyl-3-[4-(1H-pyrrolo[2,3-b]pyridin-4-yl)-1H-pyrazol-1-yl]butan-1-ol;
4-methyl-4-[4-(1H-pyrrolo[2,3-b]pyridin-4-yl)-1H-pyrazol-1-yl]pentanenitrile;
4-methyl-4-[4-(1H-pyrrolo[2,3-b]pyridin-4-yl)-1H-pyrazol-1-yl]pentanamide;
3-[4-(1H-pyrrolo[2,3-b]pyridin-4-yl)-1H-pyrazol-1-yl]butanenitrile;
3-[4-(1H-pyrrolo[2,3-b]pyridin-4-yl)-1H-pyrazol-1-yl]propanenitrile;
3-[4-(1H-pyrrolo[2,3-b]pyridin-4-yl)-1H-pyrazol-1-yl]hexanenitrile;
3-cyclopentyl-3-[4-(1H-pyrrolo[2,3-b]pyridin-4-yl)-1H-pyrazol-1-yl]propanenitrile;
3-cyclohexyl-3-[4-(1H-pyrrolo[2,3-b]pyridin-4-yl)-1H-pyrazol-1-yl]propanenitrile;
3-[4-(7H-pyrrolo[2,3-d]pyrimidin-4-yl)-1H-pyrazol-1-yl]butanenitrile;
3-cyclopentyl-3-[4-(7H-pyrrolo[2,3-d]pyrimidin-4-yl)-1H-pyrazol-1-yl]propanenitrile;
2-methyl-3-[4-(7H-pyrrolo[2,3-d]pyrimidin-4-yl)-1H-pyrazol-1-yl]propanenitrile;
3-[4-(7H-pyrrolo[2,3-d]pyrimidin-4-yl)-1H-pyrazol-1-yl]pentanenitrile;
5-methyl-3-[4-(7H-pyrrolo[2,3-d]pyrimidin-4-yl)-1H-pyrazol-1-yl]hexanenitrile;
3-cyclohexyl-3-[4-(7H-pyrrolo[2,3-d]pyrimidin-4-yl)-1H-pyrazol-1-yl]propanenitrile;
4-cyclopropyl-3-[4-(7H-pyrrolo[2,3-d]pyrimidin-4-yl)-1H-pyrazol-1-yl]butanenitrile;
4-{1-[1-methylbutyl]-1H-pyrazol-4-yl}-7H-pyrrolo[2,3-d]pyrimidine;
4-methyl-4-[4-(7H-pyrrolo[2,3-d]pyrimidin-4-yl)-1H-pyrazol-1-yl]pentanenitrile;
3-1-[4-(7H-pyrrolo[2,3-d]pyrimidin-4-yl)-1H-pyrazol-1-yl]cyclopropylpropanenitrile;
4-[4-(7H-pyrrolo[2,3-d]pyrimidin-4-yl)-1H-pyrazol-1-yl]pentanenitrile;
3-methyl-3-[4-(7H-pyrrolo[2,3-d]pyrimidin-4-yl)-1H-pyrazol-1-yl]butanenitrile;
3-ethyl-3-[4-(7H-pyrrolo[2,3-d]pyrimidin-4-yl)-1H-pyrazol-1-yl]pentanenitrile;
1-[4-(7H-pyrrolo[2,3-d]pyrimidin-4-yl)-1H-pyrazol-1-yl]cyclopropylacetonitrile;
4-1-[(pyrrolidin-2-ylmethyl]-1H-pyrazol-4-yl-7H-pyrrolo[2,3-d]pyrimidine;
4-(1-[1-(methylsulfonyl)pyrrolidin-2-yl]methyl-1H-pyrazol-4-72yl)-7H-pyrrolo[2,3-d]-
pyrimidine;
ethyl2-methyl-2-[4-(7H-pyrrolo[2,3-d]pyrimidin-4-yl)-1H-pyrazol-1-yl]propanoate;
3-cyclopentyl-3-[4-(7H-pyrrolo[2,3-d]pyrimidin-4-yl)-1H-pyrazol-1-yl]acrylonitrile;
3-cyclopentylidene-3-[4-(7H-pyrrolo[2,3-d]pyrimidin-4-yl)-1H-pyrazol-1-
yl]propanenitrile;
3-(methyl[5-(7H-pyrrolo[2,3-d]pyrimidin-4-yl)-1,3-thiazol-2-yl]amino)propanenitrile;
3-[5-(7H-pyrrolo[2,3-d]pyrimidin-4-yl)-1,3-thiazol-2-yl]hexanenitrile;
3-cyclopentyl-3-[5-(7H-pyrrolo[2,3-d]pyrimidin-4-yl)-1,3-thiazol-2-yl]propanenitrile;
5-methyl-3-[5-(7H-pyrrolo[2,3-d]pyrimidin-4-yl)-1,3-thiazol-2-yl]hexanenitrile;
3-pyridin-3-yl-3-[5-(7H-pyrrolo[2,3-d]pyrimidin-4-yl)-1,3-thiazol-2-yl]propanenitrile;
3-(5-bromopyridin-3-yl)-3-[5-(7H-pyrrolo[2,3-d]pyrimidin-4-yl)-1,3-thiazol-2-
yl]propane-nitrile;
5-(2-cyano-1-[5-(7H-pyrrolo[2,3-d]pyrimidin-4-yl)-1,3-thiazol-2-yl]ethyl)nicotinonitrile;
3-[5-(7H-pyrrolo[2,3-d]pyrimidin-4-yl)-1,3-thiazol-2-yl]butanenitrile;
2-[5-(7H-pyrrolo[2,3-d]pyrimidin-4-yl)-1,3-thiazol-2-yl]pentanenitrile;
4-[5-(7H-pyrrolo[2,3-d]pyrimidin-4-yl)-1,3-thiazol-2-yl]heptanenitrile;
3-[5-(7H-pyrrolo[2,3-d]pyrimidin-4-yl)-1,3-thiazol-2-yl]pentanedinitrile;
3-cyclopentyl-3-[5-(7H-pyrrolo[2,3-d]pyrimidin-4-yl)-1,3-oxazol-2-yl]propanenitrile;
3-[5-(7H-pyrrolo[2,3-d]pyrimidin-4-yl)-1,3-oxazol-2-yl]hexanenitrile;
5-(methylthio)-3-[4-(7H-pyrrolo[2,3-d]pyrimidin-4-yl)-1H-pyrazol-1-yl]pentanenitrile;
5-(methylsulfinyl)-3-[4-(7H-pyrrolo[2,3-d]pyrimidin-4-yl)-1H-pyrazol-1-yl]-
pentanenitrile;
5-(methylsulfonyl)-3-[4-(7H-pyrrolo[2,3-d]pyrimidin-4-yl)-1H-pyrazol-1-yl]-
pentanenitrile;
4,4,4-trifluoro-3-[4-(7H-pyrrolo[2,3-d]pyrimidin-4-yl)-pyrazol-1-yl]-butyronitrile;
5,5-dimethyl-3-[4-(7H-pyrrolo[2,3-d]pyrimidin-4-yl)-pyrazol-1-yl]-hexanenitrile;
4-[1-(2-methanesulfonyl-ethyl)-1H-pyrazol-4-yl]-7H-pyrrolo[2,3-d]pyrimidine;
5,5,5-trifluoro-4-[4-(7H-pyrrolo[2,3-d]pyrimidin-4-yl)-pyrazol-1-yl]-pentanenitrile;
3-(2-cyano-1-[4-(7H-pyrrolo[2,3-d]pyrimidin-4-yl)-1H-pyrazol-1-yl]ethyl)-cyclopentane-
carbonitrile;
3-[3-(hydroxymethyl)cyclopentyl]-3-[4-(7H-pyrrolo[2,3-d]pyrimidin-4-yl)-1H-pyrazol-
1-yl]-propanenitrile;
1-(1H-pyrrolo[2,3-b]pyridin-4-yl)-1H-indazole;
2-(1H-pyrrolo[2,3-b]pyridin-4-yl)-2H-indazole;
3-[3-(1H-pyrrolo[2,3-b]pyridin-4-yl)-1,2,4-oxadiazol-5-yl]benzonitrile;
4-(1-benzothien-2-yl)-1H-pyrrolo[2,3-b]pyridine;
4-fluoro-2-[1-(1H-pyrrolo[2,3-b]pyridin-4-yl)-1H-pyrazol-3-yl]phenol;
4-3-[3-(trifluoromethyl)phenyl]-1H-pyrazol-1-yl-1H-pyrrolo[2,3-b]pyridine;
3-[1-(1H-pyrrolo[2,3-b]pyridin-4-yl)-1H-pyrazol-3-yl]benzonitrile;
3-[1-(1H-pyrrolo[2,3-b]pyridin-4-yl)-1H-pyrazol-4-yl]benzonitrile;
2-[1-(1H-pyrrolo[2,3-b]pyridin-4-yl)-1H-pyrazol-4-yl]-1,3-benzoxazole; cyclohexyl[1-(1H-
pyrrolo[2,3-b]pyridin-4-yl)-1H-pyrazol-4-yl]methanol;
4-[4-(1-phenylvinyl)-1H-pyrazol-1-yl]-1H-pyrrolo[2,3-b]pyridine;
4-(1-benzyl-1H-pyrazol-4-yl)-1H-pyrrolo[2,3-b]pyridine;
4-[1-(2-naphthylmethyl)-1H-pyrazol-4-yl]-1H-pyrrolo[2,3-b]pyridine;
4-(1-phenyl-1H-pyrazol-4-yl)-1H-pyrrolo[2,3-b]pyridine;
3-[4-(1H-pyrrolo[2,3-b]pyridin-4-yl)-1H-pyrazol-1-yl]benzonitrile;
4-{1-[1-methylbutyl]-1H-pyrazol-4-yl}-1H-pyrrolo[2,3-b]pyridine;
4-methyl-3-[4-(1H-pyrrolo[2,3-b]pyridin-4-yl)-1H-pyrazol-1-yl]benzonitrile;
2-(1H-pyrrolo[2,3-b]pyridin-4-yl)-4,5,6,7-tetrahydro-2H-indazole;
5-nitro-2-(1H-pyrrolo[2,3-b]pyridin-4-yl)-2H-indazole;
6-nitro-2-(1H-pyrrolo[2,3-b]pyridin-4-yl)-2H-indazole;
3-[1-(1H-pyrrolo[2,3-b]pyridin-4-yl)-1H-imidazol-4-yl]benzonitrile;
4-[4-(3-methoxyphenyl)-1H-imidazol-1-yl]-1H-pyrrolo[2,3-b]pyridine;
4-(5-phenyl-2-thienyl)-1H-pyrrolo[2,3-b]pyridine;
4-[3-(4-fluorophenyl)-1H-pyrazol-1-yl]-1H-pyrrolo[2,3-b]pyridine;
4-[3-(3-nitrophenyl)-1H-pyrazol-1-yl]-1H-pyrrolo[2,3-b]pyridine;
4-[3-(4-chlorophenyl)-1H-pyrazol-1-yl]-1H-pyrrolo[2,3-b]pyridine;
4-[3-(4-methoxyphenyl)-1H-pyrazol-1-yl]-1H-pyrrolo[2,3-b]pyridine;
4-[1-(1H-pyrrolo[2,3-b]pyridin-4-yl)-1H-pyrazol-3-yl]benzonitrile;
3-[1-(1H-pyrrolo[2,3-b]pyridin-4-yl)-1H-pyrazol-3-yl]aniline;
4-[3-(3-methoxyphenyl)-1H-pyrazol-1-yl]-1H-pyrrolo[2,3-b]pyridine;
{3-[1-(1H-pyrrolo[2,3-b]pyridin-4-yl)-1H-pyrazol-3-yl]phenoxy}acetonitrile;
2-cyano-N-{3-[1-(1H-pyrrolo[2,3-b]pyridin-4-yl)-1H-pyrazol-3-yl]phenyl}acetamide;
3-cyano-N-{3-[1-(1H-pyrrolo[2,3-b]pyridin-4-yl)-1H-pyrazol-3-yl]phenyl}benzamide;
4-[4-(4-nitrophenyl)-1H-pyrazol-1-yl]-1H-pyrrolo[2,3-b]pyridine;
4-[1-(1H-pyrrolo[2,3-b]pyridin-4-yl)-1H-pyrazol-4-yl]aniline;
4-(4-phenyl-1H-pyrazol-1-yl)-1H-pyrrolo[2,3-b]pyridine;
4-(4-pyridin-3-yl-1H-pyrazol-1-yl)-1H-pyrrolo[2,3-b]pyridine;
2-[1-(1H-pyrrolo[2,3-b]pyridin-4-yl)-1H-pyrazol-4-yl]benzonitrile;
{2-[1-(1H-pyrrolo[2,3-b]pyridin-4-yl)-1H-pyrazol-4-yl]phenyl}acetonitrile;
4-[4-(3-nitrophenyl)-1H-pyrazol-1-yl]-1H-pyrrolo[2,3-b]pyridine;
3-[1-(1H-pyrrolo[2,3-b]pyridin-4-yl)-1H-pyrazol-4-yl]aniline;
{3-[1-(1H-pyrrolo[2,3-b]pyridin-4-yl)-1H-pyrazol-4-yl]phenyl}acetonitrile;
4-[1-(1H-pyrrolo[2,3-b]pyridin-4-yl)-1H-pyrazol-4-yl]benzonitrile;
3-[1-(1H-pyrrolo[2,3-b]pyridin-4-yl)-1H-pyrazol-4-yl]phenol;
methyl3-[1-(1H-pyrrolo[2,3-b]pyridin-4-yl)-1H-pyrazol-4-yl]benzoate;
{4-[1-(1H-pyrrolo[2,3-b]pyridin-4-yl)-1H-pyrazol-4-yl]phenyl}acetonitrile;
2-cyano-N-{3-[1-(1H-pyrrolo[2,3-b]pyridin-4-yl)-1H-pyrazol-4-yl]phenyl}acetamide;
4-[1-(1H-pyrrolo[2,3-b]pyridin-4-yl)-1H-pyrazol-4-yl]phenol;
5-[1-(1H-pyrrolo[2,3-b]pyridin-4-yl)-1H-pyrazol-4-yl]nicotinonitrile;
{4-[1-(1H-pyrrolo[2,3-b]pyridin-4-yl)-1H-pyrazol-4-yl]phenoxy}acetonitrile;
4-(4-cyclohex-1-en-1-yl-1H-pyrazol-1-yl)-1H-pyrrolo[2,3-b]pyridine;
4-[4-(4-methoxyphenyl)-1H-pyrazol-1-yl]-1H-pyrrolo[2,3-b]pyridine;
4-(4-pyrimidin-4-yl-1H-pyrazol-1-yl)-1H-pyrrolo[2,3-b]pyridine;
3-{hydroxy[1-(1H-pyrrolo[2,3-b]pyridin-4-yl)-1H-pyrazol-4-yl]methyl}benzonitrile;
4-[4-(cyclohex-1-en-1-ylmethyl)-1H-pyrazol-1-yl]-1H-pyrrolo[2,3-b]pyridine;
4-[1-(3,5-dimethoxybenzyl)-1H-pyrazol-4-yl]-1H-pyrrolo[2,3-b]pyridine;
4-[1-(1-phenylethyl)-1H-pyrazol-4-yl]-1H-pyrrolo[2,3-b]pyridine;
4-[1-(cyclohexylmethyl)-1H-pyrazol-4-yl]-1H-pyrrolo[2,3-b]pyridine;
3-{[4-(1H-pyrrolo[2,3-b]pyridin-4-yl)-1H-pyrazol-1-yl]methyl}benzonitrile;
2-{[4-(1H-pyrrolo[2,3-b]pyridin-4-yl)-1H-pyrazol-1-yl]methyl}benzonitrile;
4-{[4-(1H-pyrrolo[2,3-b]pyridin-4-yl)-1H-pyrazol-1-yl]methyl}benzonitrile;
1-phenyl-2-[4-(1H-pyrrolo[2,3-b]pyridin-4-yl)-1H-pyrazol-1-yl]ethanone;
3,3-dimethyl-1-[4-(1H-pyrrolo[2,3-b]pyridin-4-yl)-1H-pyrazol-1-yl]butan-2-one;
4-{1-[(5-methylisoxazol-3-yl)methyl]-1H-pyrazol-4-yl}-1H-pyrrolo[2,3-b]pyridine;
4-[1-(tetrahydro-2H-pyran-2-ylmethyl)-1H-pyrazol-4-yl]-1H-pyrrolo[2,3-b]pyridine;
4-(1-cyclohex-2-en-1-yl-1H-pyrazol-4-yl)-1H-pyrrolo[2,3-b]pyridine;
4-[1-(1-ethylpropyl)-1H-pyrazol-4-yl]-1H-pyrrolo[2,3-b]pyridine;
4-(1-cyclohexyl-1H-pyrazol-4-yl)-1H-pyrrolo[2,3-b]pyridine;
2-[4-(1H-pyrrolo[2,3-b]pyridin-4-yl)-1H-pyrazol-1-yl]acetamide;
4′-{[4-(1H-pyrrolo[2,3-b]pyridin-4-yl)-1H-pyrazol-1-yl]methyl}biphenyl-2-carbonitrile;
4-[1-(2-nitrobenzyl)-1H-pyrazol-4-yl]-1H-pyrrolo[2,3-b]pyridine;
4-{1-[2,6-dichloro-4-(trifluoromethyl)phenyl]-1H-pyrazol-4-yl}-1H-pyrrolo[2,3-
b]pyridine;
4-[1-(3-nitrobenzyl)-1H-pyrazol-4-yl]-1H-pyrrolo[2,3-b]pyridine;
4-[1-(2-bromobenzyl)-1H-pyrazol-4-yl]-1H-pyrrolo[2,3-b]pyridine;
N-phenyl-2-[4-(1H-pyrrolo[2,3-b]pyridin-4-yl)-1H-pyrazol-1-yl]propanamide;
4-{1-[3-(trifluoromethoxy)benzyl]-1H-pyrazol-4-yl}-1H-pyrrolo[2,3-b]pyridine;
4-{1-[2-fluoro-5-(trifluoromethyl)benzyl]-1H-pyrazol-4-yl}-1H-pyrrolo[2,3-b]pyridine;
4-{1-[3-(trifluoromethyl)benzyl]-1H-pyrazol-4-yl}-1H-pyrrolo[2,3-b]pyridine;
4-[1-(pyridin-3-ylmethyl)-1H-pyrazol-4-yl]-1H-pyrrolo[2,3-b]pyridine;
4-{1-[1-phenylbutyl]-1H-pyrazol-4-yl}-1H-pyrrolo[2,3-b]pyridine;
1-phenyl-2-[4-(1H-pyrrolo[2,3-b]pyridin-4-yl)-1H-pyrazol-1-yl]propan-1-one;
4-[1-(2,6-dichlorobenzyl)-1H-pyrazol-4-yl]-1H-pyrrolo[2,3-b]pyridine;
4-[1-(2,6-dimethylphenyl)-1H-pyrazol-4-yl]-1H-pyrrolo[2,3-b]pyridine;
2-[4-(1H-pyrrolo[2,3-b]pyridin-4-yl)-1H-pyrazol-1-yl]-5-(trifluoromethyl)-benzonitrile;
4-[1-(4-bromo-3,5,6-trifluoropyridin-2-yl)-1H-pyrazol-4-yl]-1H-pyrrolo[2,3-b]pyridine;
4-[1-(cyclopropylmethyl)-1H-pyrazol-4-yl]-1H-pyrrolo[2,3-b]pyridine;
4-[1-(2,5-dimethylphenyl)-1H-pyrazol-4-yl]-1H-pyrrolo[2,3-b]pyridine;
4-[1-(2-methylphenyl)-1H-pyrazol-4-yl]-1H-pyrrolo[2,3-b]pyridine;
4-[1-(2-methoxyphenyl)-1H-pyrazol-4-yl]-1H-pyrrolo[2,3-b]pyridine;
3-{1-[4-(1H-pyrrolo[2,3-b]pyridin-4-yl)-1H-pyrazol-1-yl]ethyl}benzonitrile;
3-chloro-4-[4-(1H-pyrrolo[2,3-b]pyridin-4-yl)-1H-pyrazol-1-yl]benzonitrile;
4-[1-(1-cyclohexylethyl)-1H-pyrazol-4-yl]-1H-pyrrolo[2,3-b]pyridine;
4-fluoro-2-[4-(1H-pyrrolo[2,3-b]pyridin-4-yl)-1H-pyrazol-1-yl]benzonitrile;
2-fluoro-4-[4-(1H-pyrrolo[2,3-b]pyridin-4-yl)-1H-pyrazol-1-yl]benzonitrile;
3-fluoro-4-[4-(1H-pyrrolo[2,3-b]pyridin-4-yl)-1H-pyrazol-1-yl]benzonitrile;
4-(1-{1-[3-(trifluoromethyl)phenyl]ethyl}-1H-pyrazol-4-yl)-1H-pyrrolo[2,3-b]pyridine;
4-[1-(3,5-dimethylphenyl)-1H-pyrazol-4-yl]-1H-pyrrolo[2,3-b]pyridine;
4-[4-(1H-pyrrolo[2,3-b]pyridin-4-yl)-1H-pyrazol-1-yl]benzonitrile;
{4-[4-(1H-pyrrolo[2,3-b]pyridin-4-yl)-1H-pyrazol-1-yl]phenyl}acetonitrile;
4-[1-(1-methylhexyl)-1H-pyrazol-4-yl]-1H-pyrrolo[2,3-b]pyridine;
4-(1-sec-butyl-1H-pyrazol-4-yl)-1H-pyrrolo[2,3-b]pyridine;
4-[1-(1-phenylpropyl)-1H-pyrazol-4-yl]-1H-pyrrolo[2,3-b]pyridine;
4-(1-{1-[4-(methylsulfonyl)phenyl]ethyl}-1H-pyrazol-4-yl)-1H-pyrrolo[2,3-b]pyridine;
4-{1-[I-(3-fluoro-4-methoxyphenyl)ethyl]-1H-pyrazol-4-yl}-1H-pyrrolo[2,3-b]pyridine;
4-(1-{1-[2-(trifluoromethyl)phenyl]ethyl}-1H-pyrazol-4-yl)-1H-pyrrolo[2,3-b]pyridine;
4-(1-{1-[3,5-bis(trifluoromethyl)phenyl]ethyl}-1H-pyrazol-4-yl)-1H-pyrrolo[2,3-
b]pyridine;
4-{1-[4-(1H-pyrrolo[2,3-b]pyridin-4-yl)-1H-pyrazol-1-yl]ethyl}benzonitrile;
4-{1-[4-nitro-2-(trifluoromethyl)phenyl]-1H-pyrazol-4-yl}-1H-pyrrolo[2,3-b]pyridine;
3-methyl-4-[4-(1H-pyrrolo[2,3-b]pyridin-4-yl)-1H-pyrazol-1-yl]benzonitrile;
4-[1-(2-chlorophenyl)-1H-pyrazol-4-yl]-1H-pyrrolo[2,3-b]pyridine;
3-bromo-4-[4-(1H-pyrrolo[2,3-b]pyridin-4-yl)-1H-pyrazol-1-yl]benzonitrile; ethyl4-[4-
(1H-pyrrolo[2,3-b]pyridin-4-yl)-1H-pyrazol-1-yl]benzoate;
4-{1-[2-chloro-6-nitro-4-(trifluoromethyl)phenyl]-1H-pyrazol-4-yl}-1H-pyrrolo[2,3-b]-
pyridine;
4-(1-{1-[4-(trifluoromethyl)phenyl]ethyl}-1H-pyrazol-4-yl)-1H-pyrrolo[2,3-b]pyridine;
4-[1-(2,3-dihydro-1H-inden-1-yl)-1H-pyrazol-4-yl]-1H-pyrrolo[2,3-b]pyridine;
4-[1-(1,2,3,4-tetrahydronaphthalen-1-yl)-1H-pyrazol-4-yl]-1H-pyrrolo[2,3-b]pyridine;
4-(1-{1-[2-chloro-5-(trifluoromethyl)phenyl]ethyl}-1H-pyrazol-4-yl)-1H-pyrrolo[2,3-b]-
pyridine;
4-{1-[1-(2,4-dichloro-5-fluorophenyl)ethyl]-1H-pyrazol-4-yl}-1H-pyrrolo[2,3-b]pyridine;
4-[1-(1-cyclopentylethyl)-1H-pyrazol-4-yl]-1H-pyrrolo[2,3-b]pyridine;
4-[1-(1-methyl-3-phenylpropyl)-1H-pyrazol-4-yl]-1H-pyrrolo[2,3-b]pyridine;
4-[1-(1-cyclobutylethyl)-1H-pyrazol-4-yl]-1H-pyrrolo[2,3-b]pyridine;
[2-[4-(1H-pyrrolo[2,3-b]pyridin-4-yl)-1H-pyrazol-1-yl]-5-(trifluoromethyl)phenyl]-
acetonitrile;
[5-[4-(1H-pyrrolo[2,3-b]pyridin-4-yl)-1H-pyrazol-1-yl]-2-(trifluoromethyl)phenyl]-
acetonitrile;
4-{1-[pent-3-en-1-yl]-1H-pyrazol-4-yl}-1H-pyrrolo[2,3-b]pyridine;
2-[4-(1H-pyrrolo[2,3-b]pyridin-4-yl)-1H-pyrazol-1-yl]propanenitrile;
4-{1-[4-phenylbut-3-en-1-yl]-1H-pyrazol-4-yl}-1H-pyrrolo[2,3-b]pyridine;
6-[4-(1H-pyrrolo[2,3-b]pyridin-4-yl)-1H-pyrazol-1-yl]hexanenitrile;
ethyl3-amino-2-{[4-(1H-pyrrolo[2,3-b]pyridin-4-yl)-1H-pyrazol-1-yl]methyl}-
propanoate;
ethyl2-[4-(1H-pyrrolo[2,3-b]pyridin-4-yl)-1H-pyrazol-1-yl]propanoate;
4-[1-(1-propylbutyl)-1H-pyrazol-4-yl]-1H-pyrrolo[2,3-b]pyridine;
4-[4-(1H-pyrrolo[2,3-b]pyridin-4-yl)-1H-pyrazol-1-yl]butanenitrile;
[3-chloro-2-[4-(1H-pyrrolo[2,3-b]pyridin-4-yl)-1H-pyrazol-1-yl]-5-
(trifluoromethyl)phenyl]-acetonitrile;
5-[4-(1H-pyrrolo[2,3-b]pyridin-4-yl)-1H-pyrazol-1-yl]-2-(trifluoromethyl)-benzonitrile;
4-{1-[2-chloro-4-(trifluoromethyl)phenyl]-1H-pyrazol-4-yl}-1H-pyrrolo[2,3-b]pyridine;
4-[4-(1H-pyrrolo[2,3-b]pyridin-4-yl)-1H-pyrazol-1-yl]-2-(trifluoromethyl)-benzonitrile;
2-[4-(1H-pyrrolo[2,3-b]pyridin-4-yl)-1H-pyrazol-1-yl]benzonitrile;
3-chloro-2-[4-(1H-pyrrolo[2,3-b]pyridin-4-yl)-1H-pyrazol-1-yl]benzonitrile;
4-amino-5,6-difluoro-2-[4-(1H-pyrrolo[2,3-b]pyridin-4-yl)-1H-pyrazol-1-
yl]isophthalonitrile;
1-{[4-(1H-pyrrolo[2,3-b]pyridin-4-yl)-1H-pyrazol-1-yl]methyl}-
cyclopropanecarbonitrile;
5-[4-(1H-pyrrolo[2,3-b]pyridin-4-yl)-1H-pyrazol-1-yl]hexanenitrile;
2,2-dimethyl-6-[4-(1H-pyrrolo[2,3-b]pyridin-4-yl)-1H-pyrazol-1-yl]hexanenitrile;
4-[1-(1-ethyl-2-methylpropyl)-1H-pyrazol-4-yl]-1H-pyrrolo[2,3-b]pyridine;
5-bromo-2-[4-(1H-pyrrolo[2,3-b]pyridin-4-yl)-1H-pyrazol-1-yl]benzonitrile;
3-[4-(1H-pyrrolo[2,3-b]pyridin-4-yl)-1H-pyrazol-1-yl]-4-(trifluoromethyl)-benzonitrile;
2-[4-(1H-pyrrolo[2,3-b]pyridin-4-yl)-1H-pyrazol-1-yl]-3-(trifluoromethyl)-benzonitrile;
3-[4-(1H-pyrrolo[2,3-b]pyridin-4-yl)-1H-pyrazol-1-yl]-4-(trifluoromethyl)benzamide;
3-[4-(1H-pyrrolo[2,3-b]pyridin-4-yl)-1H-pyrazol-1-yl]cyclohexanone;
2-[4-(1H-pyrrolo[2,3-b]pyridin-4-yl)-1H-pyrazol-1-yl]cyclohexanol;
4-(1-{[1-(methylsulfonyl)piperidin-4-yl]methyl}-1H-pyrazol-4-yl)-1H-pyrrolo[2,3-b]-
pyridine;
2-[4-(1H-pyrrolo[2,3-b]pyridin-4-yl)-1H-pyrazol-1-yl]cyclohexanecarbonitrile;
4-{1-[2-(trifluoromethyl)phenyl]-1H-pyrazol-4-yl}-1H-pyrrolo[2,3-b]pyridine;
4-[1-(2,6-dichlorophenyl)-1H-pyrazol-4-yl]-1H-pyrrolo[2,3-b]pyridine;
(4-{[4-(1H-pyrrolo[2,3-b]pyridin-4-yl)-1H-pyrazol-1-yl]methyl}cyclohexyl)methanol;
4-[1-(tetrahydrofuran-2-ylmethyl)-1H-pyrazol-4-yl]-1H-pyrrolo[2,3-b]pyridine;
4-[1-(1-cyclopentylpropyl)-1H-pyrazol-4-yl]-1H-pyrrolo[2,3-b]pyridine;
4-[1-(tetrahydroruran-3-ylmethyl)-1H-pyrazol-4-yl]-1H-pyrrolo[2,3-b]pyridine;
2-chloro-3-[4-(1H-pyrrolo[2,3-b]pyridin-4-yl)-1H-pyrazol-1-yl]benzonitrile;
3-[4-(1H-pyrrolo[2,3-b]pyridin-4-yl)-1H-pyrazol-1-yl]-3-(1,3-thiazol-5-yl)propanenitrile;
1-benzyl-4-{[4-(1H-pyrrolo[2,3-b]pyridin-4-yl)-1H-pyrazol-1-yl]methyl}pyrrolidin-2-one;
3-(1-methyl-1H-imidazol-5-yl)-3-[4-(1H-pyrrolo[2,3-b]pyridin-4-yl)-1H-pyrazol-1-yl]-
propanenitrile;
3-[4-(1H-pyrrolo[2,3-b]pyridin-4-yl)-1H-pyrazol-1-yl]-3-(3-thienyl)propanenitrile;
{1-[4-(1H-pyrrolo[2,3-b]pyridin-4-yl)-1H-pyrazol-1-yl]cyclopentyl}acetonitrile;
4-chloro-3-[4-(1H-pyrrolo[2,3-b]pyridin-4-yl)-1H-pyrazol-1-yl]benzonitrile;
4-[4-(1H-pyrrolo[2,3-b]pyridin-4-yl)-1H-pyrazol-1-yl]phthalonitrile;
3-methyl-4-[4-(1H-pyrrolo[2,3-b]pyridin-4-yl)-1H-pyrazol-1-yl]benzaldehyde;
4-[1-(2-methyl-4-nitrophenyl)-1H-pyrazol-4-yl]-1H-pyrrolo[2,3-b]pyridine;
3-[4-(1H-pyrrolo[2,3-b]pyridin-4-yl)-1H-pyrazol-1-yl]cyclopentanone;
4-[1-(3-furylmethyl)-1H-pyrazol-4-yl]-1H-pyrrolo[2,3-b]pyridine;
4-[1-(2-furylmethyl)-1H-pyrazol-4-yl]-1H-pyrrolo[2,3-b]pyridine;
3-{2-cyano-1-[4-(1H-pyrrolo[2,3-b]pyridin-4-yl)-1H-pyrazol-1-yl]ethyl}benzonitrile;
{3-methyl-4-[4-(1H-pyrrolo[2,3-b]pyridin-4-yl)-1H-pyrazol-1-yl]phenyl}methanol;
4-methyl-4-[4-(1H-pyrrolo[2,3-b]pyridin-4-yl)-1H-pyrazol-1-yl]pentan-2-one;
3-(1-benzofuran-2-yl)-3-[4-(1H-pyrrolo[2,3-b]pyridin-4-yl)-1H-pyrazol-1-
yljpropanenitrile;
3-(3-furyl)-3-[4-(1H-pyrrolo[2,3-b]pyridin-4-yl)-1H-pyrazol-1-yl]propanenitrile;
{3-methyl-4-[4-(1H-pyrrolo[2,3-b]pyridin-4-yl)-1H-pyrazol-1-yl]phenyl}acetonitrile;
4-methyl-3-[4-(7H-pyrrolo[2,3-d]pyrimidin-4-yl)-1H-pyrazol-1-yl]benzonitrile;
4-[1-(1-cyclopentylpropyl)-1H-pyrazol-4-yl]-7H-pyrrolo[2,3-d]pyrimidine;
{1-[4-(7H-pyrrolo[2,3-d]pyrimidin-4-yl)-1H-pyrazol-1-yl]cyclopentyl}acetonitrile;
3-{(2-cyano-1-[4-(7H-pyrrolo[2,3-d]pyrimidin-4-yl)-1H-pyrazol-1-yl]ethyl}benzonitrile;
3-[4-(7H-pyrrolo[2,3-d]pyrimidin-4-yl)-1H-pyrazol-1-yl]-3-(3-thienyl)propanenitrile;
4-chloro-3-[4-(7H-pyrrolo[2,3-d]pyrimidin-4-yl)-1H-pyrazol-1-yl]benzonitrile;
3-(3-furyl)-3-[4-(7H-pyrrolo[2,3-d]pyrimidin-4-yl)-1H-pyrazol-1-yl]propanenitrile;
3-[4-(7H-pyrrolo[2,3-d]pyrimidin-4-yl)-1H-pyrazol-1-yl]pentanedinitrile;
3-{1-[4-(7H-pyrrolo[2,3-d]pyrimidin-4-yl)-1H-pyrazol-1-yl]cyclopentyl}-propanenitrile;
{1-[4-(7H-pyrrolo[2,3-d]pyrimidin-4-yl)-1H-pyrazol-1-yl]cyclohexyl}acetonitrile;
{3-methyl-4-[4-(7H-pyrrolo[2,3-d]pyrimidin-4-yl)-1H-pyrazol-1-yl]phenyl}methanol;
3-pyridin-4-yl-3-[4-(7H-pyrrolo[2,3-d]pyrimidin-4-yl)-1H-pyrazol-1-yl]propanenitrile;
3-pyridin-3-yl-3-[4-(7H-pyrrolo[2,3-d]pyrimidin-4-yl)-1H-pyrazol-1-yl]propanenitrile;
3-[4-(methylthio)phenyl]-3-[4-(7H-pyrrolo[2,3-d]pyrimidin-4-yl)-1H-pyrazol-1-
yl]propane-nitrile;
3-(3-methoxyphenyl)-3-[4-(7H-pyrrolo[2,3-d]pyrimidin-4-yl)-1H-pyrazol-1-
yl]propanenitrile;
3-(4-methoxypheny)-3-[4-(7H-pyrrolo[2,3-d]pyrimidin-4-yl)-1H-pyrazol-1-
yl]propanenitrile;
{3-methyl-4-[4-(7H-pyrrolo[2,3-d]pyrimidin-4-yl)-1H-pyrazol-1-yl]phenyl}acetonitrile;
3-[4-(methylsulfinyl)phenyl]-3-[4-(7H-pyrrolo[2,3-d]pyrimidin-4-yl)-1H-pyrazol-1-yl]-
propanenitrile;
3-[4-(methylsulfonyl)phenyl]-3-[4-(7H-pyrrolo[2,3-d]pyrimidin-4-yl)-1H-pyrazol-1-yl]-
propanenitrile;
3-[3-(cyanomethoxy)phenyl]-3-[4-(7H-pyrrolo[2,3-d]pyrimidin-4-yl)-1H-pyrazol-1-yl]-
propanenitrile;
3-(6-chloropyridin-3-yl)-3-[4-(7H-pyrrolo[2,3-d]pyrimidin-4-yl)-1H-pyrazol-1-yl]-
propanenitrile;
5-{2-cyano-1-[4-(7H-pyrrolo[2,3-d]pyrimidin-4-yl)-1H-pyrazol-1-yl]ethyl}pyridine-2-
carbonitrile;
3-(3,5-dimethylisoxazol-4-yl)-3-[4-(7H-pyrrolo[2,3-d]pyrimidin-4-yl)-1H-pyrazol-1-yl]-
propanenitrile;
3-[4-(7H-pyrrolo[2,3-d]pyrimidin-4-yl)-1H-pyrazol-1-yl]-3-[6-(trifluoromethyl)pyridin-3-
yl]-propanenitrile;
3-(6-methoxypyridin-3-yl)-3-[4-(7H-pyrrolo[2,3-d]pyrimidin-4-yl)-1H-pyrazol-1-
yl]propane-nitrile;
3-pyridin-2-yl-3-[4-(7H-pyrrolo[2,3-d]pyrimidin-4-yl)-1H-pyrazol-1-yl]propanenitrile;
3-(6-bromopyridin-2-yl)-3-[4-(7H-pyrrolo[2,3-d]pyrimidin-4-yl)-1H-pyrazol-1-
yl]propane-nitrile;
6-{2-cyano-1-[4-(7H-pyrrolo[2,3-d]pyrimidin-4-yl)-1H-pyrazol-1-yl]ethyl}pyridine-2-
carbonitrile;
4-[4-(7H-pyrrolo[2,3-d]pyrimidin-4-yl)-1H-pyrazol-1-yl]heptanedinitrile;
3-(5-bromopyridin-3-yl)-3-[4-(7H-pyrrolo[2,3-d]pyrimidin-4-yl)-1H-pyrazol-1-
yl]propane-nitrile;
3-[4-(7H-pyrrolo[2,3-d]pyrimidin-4-yl)-1H-pyrazol-1-yl]pentane-1,5-diol;
5-{2-cyano-1-[4-(7H-pyrrolo[2,3-d]pyrimidin-4-yl)-1H-pyrazol-1-yl]ethyl}nicotinonitrile;
3-(2-methoxypyridin-3-yl)-3-[4-(7H-pyrrolo[2,3-d]pyrimidin-4-yl)-1H-pyrazol-1-
yl]propane-nitrile;
3-[4-(cyanomethoxy)phenyl]-3-[4-(7H-pyrrolo[2,3-d]pyrimidin-4-yl)-1H-pyrazol-1-yl]-
propanenitrile;
3-[2-(cyanomethoxy)phenyl]-3-[4-(7H-pyrrolo[2,3-d]pyrimidin-4-yl)-1H-pyrazol-1-yl]-
propanenitrile;
3-(3,5-dibromophenyl)-3-[4-(7H-pyrrolo[2,3-d]pyrimidin-4-yl)-1H-pyrazol-1-yl]propane-
nitrile;
5-{2-cyano-1-[4-(7H-pyrrolo[2,3-d]pyrimidin-4-yl)-1H-pyrazol-1-
yl]ethyl}isophthalonitrile;
3-[6-(dimethylamino)pyridin-2-yl]-3-[4-(7H-pyrrolo[2,3-d]pyrimidin-4-yl)-1H-pyrazol-
1-yl]-propanenitrile;
3-(4-bromo-2-thienyl)-3-[4-(7H-pyrrolo[2,3-d]pyrimidin-4-yl)-1H-pyrazol-1-yl]propane-
nitrile;
5-{2-cyano-1-[4-(7H-pyrrolo[2,3-d]pyrimidin-4-yl)-1H-pyrazol-1-yl]ethyl}thiophene-3-
carbonitrile;
3-(5-bromo-2-fluorophenyl)-3-[4-(7H-pyrrolo[2,3-d]pyrimidin-4-yl)-1H-pyrazol-1-yl]-
propanenitrile;
3-(3-nitrophenyl)-3-[4-(7H-pyrrolo[2,3-d]pyrimidin-4-yl)-1H-pyrazol-1-yl]propanenitrile;
3-(5-bromo-2-methoxyphenyl)-3-[4-(7H-pyrrolo[2,3-d]pyrimidin-4-yl)-1H-pyrazol-1-yl]-
propanenitrile;
3-{2-cyano-1-[4-(7H-pyrrolo[2,3-d]pyrimidin-4-yl)-1H-pyrazol-1-yl]ethyl}-4-
methoxybenzonitrile;
3-(3-bromophenyl)-3-[4-(7H-pyrrolo[2,3-d]pyrimidin-4-yl)-1H-pyrazol-1-
yl]propanenitrile;
3-{2-cyano-1-[4-(7H-pyrrolo[2,3-d]pyrimidin-4-yl)-1H-pyrazol-1-yl]ethyl}-4-
fluorobenzonitrile;
3-[5-bromo-2-(cyanomethoxy)phenyl]-3-[4-(7H-pyrrolo[2,3-d]pyrimidin-4-yl)-1H-
pyrazol-1-yl]propanenitrile;
3-(4-bromo-2-fiαryl)-3-[4-(7H-pyrrolo[2,3-d]pyrimidm-4-yl)-1H-pyrazol-1-
yl]propanenitrile;
4-(cyanomethoxy)-3-{2-cyano-1-[4-(7H-pyrrolo[2,3-d]pyrimidin-4-yl)-1H-pyrazol-1-yl]-
ethyl}benzonitrile;
3-(4-bromopyridin-2-yl)-3-[4-(7H-pyrrolo[2,3-d]pyrimidin-4-yl)-1H-pyrazol-1-
yl]propanenitrile;
2-{2-cyano-1-[4-(7H-pyrrolo[2,3-d]pyrimidin-4-yl)-1H-pyrazol-1-
yl]ethyl}isonicotinonitrile;
5-{2-cyano-1-[4-(7H-pyrrolo[2,3-d]pyrimidin-4-yl)-1H-pyrazol-1-yl]ethyl}-3-furonitrile;
3-[2-bromo-5-(cyanomethoxy)phenyl]-3-[4-(7H-pyrrolo[2,3-d]pyrimidin-4-yl)-1H-
pyrazol-1-yl]propanenitrile;
4-(cyanomethoxy)-2-{2-cyano-1-[4-(7H-pyrrolo[2,3-d]pyrimidin-4-yl)-1H-pyrazol-1-yl]-
ethyl}benzonitrile;
3-pyrimidin-5-yl-3-[4-(7H-pyrrolo[2,3-d]pyrimidin-4-yl)-1H-pyrazol-1-yl]propanenitrile;
3-(2-bromopyridin-4-yl)-3-[4-(7H-pyrrolo[2,3-d]pyrimidin-4-yl)-1H-pyrazol-1-
yl]propanenitrile;
4-{2-cyano-1-[4-(7H-pyrrolo[2,3-d]pyrimidin-4-yl)-1H-pyrazol-1-yl]ethyl}pyridine-2-
carbonitrile;
3-(5-methoxypyridin-3-yl)-3-[4-(7H-pyrrolo[2,3-d]pyrimidin-4-yl)-1H-pyrazol-1-
yl]propanenitrile;
3-(3-chlorophenyl)-3-[4-(7H-pyrrolo[2,3-d]pyrimidin-4-yl)-1H-pyrazol-1-
yl]propanenitrile;
3-[4-(7H-pyrrolo[2,3-d]pyrimidin-4-yl)-1H-pyrazol-1-yl]-3-[3-
(trifluoromethyl)phenyl]-propanenitrile;
3-(3-phenoxyphenyl)-3-[4-(7H-pyrrolo[2,3-d]pyrimidin-4-yl)-1H-pyrazol-1-
yl]propanenitrile;
3-[4-(7H-pyrrolo[2,3-d]pyrimidin-4-yl)-1H-pyrazol-1-yl]-3-[3-(trifluoromethoxy)-
phenyl]propanenitrile;
methyl3-{2-cyano-1-[4-(7H-pyrrolo[2,3-d]pyrimidin-4-yl)-1H-pyrazol-1-
yl]ethyl}benzoate;
3-{2-cyano-1-[4-(7H-pyrrolo[2,3-d]pyrimidin-4-yl)-1H-pyrazol-1-yl]ethyl}benzoic acid;
3-[3-(1H-pyrazol-4-yl)phenyl]-3-[4-(7H-pyrrolo[2,3-d]pyrimidin-4-yl)-1H-pyrazol-1-yl]-
propanenitrile;
3-(3-aminophenyl)-3-[4-(7H-pyrrolo[2,3-d]pyrimidin-4-yl)-1H-pyrazol-1-
yl]propanenitrile;
N-(3-{2-cyano-1-[4-(7H-pyrrolo[2,3-d]pyrimidin-4-yl)-1H-pyrazol-1-yl]ethyl}phenyl)-
acetamide;
N-(3-{2-cyano-1-[4-(7H-pyrrolo[2,3-d]pyrimidin-4-yl)-1H-pyrazol-1-yl]ethyl}phenyl)-
methanesulfonamide;
4-{2-cyano-1-[4-(7H-pyrrolo[2,3-d]pyrimidin-4-yl)-1H-pyrazol-1-yl]ethyl}thiophene-2-
carbonitrile;
5-{2-cyano-1-[4-(7H-pyrrolo[2,3-d]pyrimidin-4-yl)-1H-pyrazol-1-yl]ethyl}thiophene-2-
carbonitrile;
3-[3-(morpholin-4-ylcarbonyl)phenyl]-3-[4-(7H-pyrrolo[2,3-d]pyrimidin-4-yl)-1H-
pyrazol-1-yl]propanenitrile;
N-(2-aminoethyl)-3-{2-cyano-1-[4-(7H-pyrrolo[2,3-d]pyrimidin-4-yl)-1H-pyrazol-1-
yl]-ethyl}benzamide;
3-(5-formyl-3-thienyl)-3-[4-(7H-pyrrolo[2,3-d]pyrimidin-4-yl)-1H-pyrazol-1-yl]propane-
nitrile;
3-{2-cyano-1-[4-(7H-pyrrolo[2,3-d]pyrimidin-4-yl)-1H-pyrazol-1-yl]ethyl}-N-
methylbenzamide;
2-cyano-N-(3-{2-cyano-1-[4-(7H-pyrrolo[2,3-d]pyrimidin-4-yl)-1H-pyrazol-1-yl]ethyl}-
phenyl)acetamide;
N-(3-{2-cyano-1-[4-(7H-pyrrolo[2,3-d]pyrimidin-4-yl)-1H-pyrazol-1-yl]ethyl}phenylnicotinamide;
N-(3-{2-cyano-1-[4-(7H-pyrrolo[2,3-d]pyrimidin-4-yl)-1H-pyrazol-1-yl]ethyl}phenyl)-
N′-isopropylurea;
isopropyl(3-{2-cyano-1-[4-(7H-pyrrolo[2,3-d]pyrimidin-4-yl)-1H-pyrazol-1-yl]ethyl}-
phenyl)carbamate;
3-(5-phenylpyridin-3-yl)-3-[4-(7H-pyrrolo[2,3-d]pyrimidin-4-yl)-1H-pyrazol-1-
yl]propanenitrile;
3-(3,3′-bipyridin-5-yl)-3-[4-(7H-pyrrolo[2,3-d]pyrimidin-4-yl)-1H-pyrazol-1-yl]propane-
nitrile;
3-(5-pyrimidin-5-ylpyridin-3-yl)-3-[4-(7H-pyrrolo[2,3-d]pyrimidin-4-yl)-1H-pyrazol-1-
yl]-propanenitrile;
3-[5-(1-methyl-1H-pyrazol-4-yl)pyridin-3-yl]-3-[4-(7H-pyrrolo[2,3-d]pyrimidin-4-yl)-1H-
pyrazol-1-yl]propanenitrile;
3-(5-ethynylpyridin-3-yl)-3-[4-(7H-pyrrolo[2,3-d]pyrimidin-4-yl)-1H-pyrazol-1-
yl]propanenitrile;
3-[5-(phenylthio)pyridin-3-yl]-3-[4-(7H-pyrrolo[2,3-d]pyrimidin-4-yl)-1H-pyrazol-1-yl]-
propanenitrile;
3-(2-bromo-1,3-thiazol-5-yl)-3-[4-(7H-pyrrolo[2,3-d]pyrimidin-4-yl)-1H-pyrazol-1-yl]-
propanenitrile;
ethyl3-[4-(7H-pyrrolo[2,3-d]pyrimidin-4-yl)-1H-pyrazol-1-yl]butanoate;
3-(5-morpholin-4-ylpyridin-3-yl)-3-[4-(7H-pyrrolo[2,3-d]pyrimidin-4-yl)-1H-pyrazol-1-
yl]-propanenitrile;
3-(1-methyl-1H-pyrazol-4-yl)-3-[4-(7H-pyrrolo[2,3-d]pyrimidin-4-yl)-1H-pyrazol-1-yl]-
propanenitrile;
4-{1-[1-phenyl-2-(1H-1,2,4-triazol-1-yl)ethyl]-1H-pyrazol-4-yl}-7H-pyrrolo[2,3-d]-
pyrimidine;
4-{1-[1-phenyl-2-(4H-1,2,4-triazol-4-yl)ethyl]-1H-pyrazol-4-yl}-7H-pyrrolo[2,3-d]-
pyrimidine;
3-(3-pyridin-3-ylphenyl)-3-[4-(7H-pyrrolo[2,3-d]pyrimidin-4-yl)-1H-pyrazol-1-yl]-
propanenitrile;
3-[5-(phenylsulfinyl)pyridin-3-yl]-3-[4-(7H-pyrrolo[2,3-d]pyrimidin-4-yl)-1H-pyrazol-1-
yl]-propanenitrile;
3-[5-(phenylsulfonyl)pyridin-3-yl]-3-[4-(7H-pyrrolo[2,3-d]pyrimidin-4-yl)-1H-pyrazol-
1-yl]-propanenitrile;
3-[4-(7H-pyrrolo[2,3-d]pyrimidin-4-yl)-1H-pyrazol-1-yl]pentan-1-ol;
methyl3-[4-(7H-pyrrolo[2,3-d]pyrimidin-4-yl)-1H-pyrazol-1-yl]pentyl carbonate;
(1E)-3-[4-(7H-pyrrolo[2,3-d]pyrimidin-4-yl)-1H-pyrazol-1-yl]pentanal oxime;
(1E)-3-[4-(7H-pyrrolo[2,3-d]pyrimidin-4-yl)-1H-pyrazol-1-yl]pentanal O-methyloxime;
(1Z)-3-[4-(7H-pyrrolo[2,3-d]pyrimidin-4-yl)-1H-pyrazol-1-yl]pentanal O-methyloxime;
4-[1-(4,4-dibromo-1-ethylbut-3-en-1-yl)-1H-pyrazol-4-yl]-7H-pyrrolo[2,3-d]pyrimidine;
3-[4-(7H-pyrrolo[2,3-d]pyrimidin-4-yl)-1H-pyrazol-1-yl]-3-[5-(1,3-thiazol-2-
ylthio)pyridin-3-yl]propanenitrile;
3-[5-(ethylthio)pyridin-3-yl]-3-[4-(7H-pyrrolo[2,3-d]pyrimidin-4-yl)-1H-pyrazol-1-yl]-
propanenitrile;
4-[1-(1-ethylbut-3-yn-1-yl)-1H-pyrazol4-yl]-7H-pyrrolo[2,3-d]pyrimidine
trifluoroacetate;
4-{1-[1-methyl-2-(1H-1,2,4-triazol-1-yl)ethyl]-1H-pyrazol-4-yl}-7H-pyrrolo[2,3-d]-
pyrimidine;
4-[4-(7H-pyrrolo[2,3-d]pyrimidin-4-yl)-1H-pyrazol-1-yl]pentan-2-one trifluoroacetate;
1-phenyl-2-[4-(7H-pyrrolo[2,3-d]pyrimidin-4-yl)-1H-pyrazol-1-yl]propan-1-one;
3-[5-(ethylsulfinyl)pyridin-3-yl]-3-[4-(7H-pyrrolo[2,3-d]pyrimidin-4-yl)-1H-pyrazol-1-
yl]-propanenitrile;
3-[5-(ethylsulfonyl)pyridin-3-yl]-3-[4-(7H-pyrrolo[2,3-d]pyrimidin-4-yl)-1H-pyrazol-1-
yl]-propanenitrile;
3-[5-(cyclohexylthio)pyridin-3-yl]-3-[4-(7H-pyrrolo[2,3-d]pyrimidin-4-yl)-1H-pyrazol-
1-yl]-propanenitrile;
1-phenyl-2-[4-(7H-pyrrolo[2,3-d]pyrimidin-4-yl)-1H-pyrazol-1-yl]propan-1-ol;
1-phenyl-2-[4-(7H-pyrrolo[2,3-d]pyrimidin-4-yl)-1H-pyrazol-1-yl]propan-1-ol;
3-[3-(ethylthio)phenyl]-3-[4-(7H-pyrrolo[2,3-d]pyrimidin-4-yl)-1H-pyrazol-1-yl]-
propanenitrile;
3-[3-(ethylsulfinyl)phenyl]-3-[4-(7H-pyrrolo[2,3-d]pyrimidin-4-yl)-1H-pyrazol-1-yl]-
propanenitrile;
3-[3-(ethylsulfonyl)phenyl]-3-[4-(7H-pyrrolo[2,3-d]pyrimidin-4-yl)-1H-pyrazol-1-yl]-
propanenitrile;
3-[5-(cyclohexylsulfonyl)pyridin-3-yl]-3-[4-(7H-pyrrolo[2,3-d]-pyrimidin-4-yl)-1H-
pyrazol-1-yl]propanenitrile;
3-[5-(cyclohexylsulfonyl)pyridin-3-yl]-3-[4-(7H-pyrrolo[2,3-d]pyrimidin-4-yl)-1H-
pyrazol-1-yl]propanenitrile;
4-[1-(1-methyl-2-phenylethyl)-1H-pyrazol-4-yl]-7H-pyrrolo[2,3-d]pyrimidine;
4-{1-[-methyl-2-(3-thienyl)ethyl]-1H-pyrazol-4-yl}-7H-pyrrolo[2,3-d]pyrimidine;
3-{1-[4-(7H-pyrrolo[2,3-d]pyrimidin-4-yl)-1H-pyrazol-1-yl]ethyl}benzonitrile;
4-{1-[2-(1H-imidazol-1-yl)-1-methylethyl]-1H-pyrazol-4-yl}-7H-pyrrolo[2,3-
d]pyrimidine;
4-{1-[1-methyl-2-(3-methyl-1,2,4-oxadiazol-5-yl)ethyl]-1H-pyrazol-4-yl}-7H-pyrrolo
[2,3-d]-pyrimidine;
3-[3-(methylsulfbnyl)phenyl]-3-[4-(7H-pyrrolo[2,3-d]pyrimidin-4-yl)-1H-pyrazol-1-yl]-
propanenitrile;
3-(3-pyridin-4-ylphenyl)-3-[4-(7H-pyrrolo[2,3-d]pyrimidin-4-yl)-1H-pyrazol-1-
yl]propane-nitrile;
4-[1-(1-ethylbut-3-en-1-yl)-1H-pyrazol-4-yl]-7H-pyrrolo[2,3-d]pyrimidine;
4-[1-(1,3-dimethylbut-3-en-1-yl)-1H-pyrazol-4-yl]-7H-pyrrolo[2,3-d]pyrimidine;
3-[5-(isopropylthio)pyridin-3-yl]-3-[4-(7H-pyrrolo[2,3-d]pyrimidin-4-yl)-1H-pyrazol-1-
yl]-propanenitrile;
3-[5-(isopropylsulfonyl)pyridin-3-yl]-3-[4-(7H-pyrrolo[2,3-d]pyrimidin-4-yl)-1H-
pyrazol-1-yl]propanenitrile;
3-[5-(isopropylsulfinyl)pyridm-3-yl]-3-[4-(7H-pyrrolo[2,3-d]pyrimidin-4-yl)-1H-
pyrazol-1-yl]propanenitrile;
3-[4-(7H-pyrrolo[2,3-d]pyrimidin-4-yl)-1H-pyrazol-1-yl]-3-[5-(trifluoromethyl)pyridin-3-
yl]-propanenitrile;
3-[4-(7H-pyrrolo[2,3-d]pyrimidin-4-yl)-1H-pyrazol-1-yl]-3-[5-(trifluoromethyl)pyridin-3-
yl]-propanenitrile;
2-[4-(7H-pyrrolo[2,3-d]pyrimidin-4-yl)-1H-pyrazol-1-yl]-N-[3-(trifluoromethyl)phenyl]-
propanamide;
N-2-naphthyl-2-[4-(7H-pyrrolo[2,3-d]pyrimidin-4-yl)-1H-pyrazol-1-yl]propanamide;
N-1-naphthyl-2-[4-(7H-pyrrolo[2,3-d]pyrimidin-4-yl)-1H-pyrazol-1-yl]propanamide;
N-(3-cyanophenyl)-2-[4-(7H-pyrrolo[2,3-d]pyrimidin-4-yl)-1H-pyrazol-1-
yl]propanamide;
N-benzyl-2-[4-(7H-pyrrolo[2,3-d]pyrimidin-4-yl)-1H-pyrazol-1-yl]propanamide;
N-phenyl-2-[4-(7H-pyrrolo[2,3-d]-′pyrimidin-4-yl)-1H-pyrazol-1-yl]-′butanamide;
N-(4-phenoxyphenyl)-2-[4-(7H-pyrrolo[2,3-d]pyrimidin-4-yl)-1H-pyrazol-1-
yl]butanamide;
N-2-naphthyl-2-[4-(7H-pyrrolo[2,3-d]pyrimidin-4-yl)-1H-pyrazol-1-yl]butanamide;
N-(3-cyanophenyl)-2-[4-(7H-pyrrolo[2,3-d]pyrimidin-4-yl)-1H-pyrazol-1-yl]butanamide;
N-biphenyl-4-yl-2-[4-(7H-pyrrolo[2,3-d]pyrimidin-4-yl)-1H-pyrazol-1-yl]butanamide;
N-(biphenyl-4-ylmethyl)-2-[4-(7H-pyrrolo[2,3-d]pyrimidin-4-yl)-1H-pyrazol-1-yl]-
butanamide;
N-(biphenyl-3-ylmethyl)-2-[4-(7H-pyrrolo[2,3-d]pyrimidin-4-yl)-1H-pyrazol-1-yl]-
butanamide;
N-(4-cyanophenyl)-2-[4-(7H-pyrrolo[2,3-d]pyrimidin-4-yl)-1H-pyrazol-1-yl]butanamide;
N-1-naphthyl-2-[4-(7H-pyrrolo[2,3-d]pyrimidin-4-yl)-1H-pyrazol-1-yl]butanamide;
5-{2-cyano-1-[4-(7H-pyrrolo[2,3-d]pyrimidin-4-yl)-1H-pyrazol-1-yl]ethyl}-N-
phenylnicotinamide;
N-(3-{2-cyano-1-[4-(7H-pyrrolo[2,3-d]pyrimidin-4-yl)-1H-pyrazol-1-yl]ethyl}phenyl)-3-
(trifluoromethyl)benzamide;
N-(3-{[4-(7H-pyrrolo[2,3-d]pyrimidin-4-yl)-1H-pyrazol-1-yl]methyl}phenyl)-3-(trifluoro-
methyl)benzamide;
3-[3-(methylsulfonyl)phenyl]-3-[4-(7H-pyrrolo[2,3-d]pyrimidin-4-yl)-1H-pyrazol-1-yl]-
propanenitrile;
3-[3-(methylsulfonyl)phenyl]-3-[4-(7H-pyrrolo[2,3-d]pyrimidin-4-yl)-1H-pyrazol-1-
yl]-propanenitrile;
N-(3-{[4-(7H-pyrrolo[2,3-d]pyrimidin-4-yl)-1H-pyrazol-1-yl]methyl}phenyl)benzene-
sulfonamide;
3-{[4-(7H-pyrrolo[2,3-d]pyrimidin-4-yl)-1H-pyrazol-1-yl]methyl}-N-[3-(trifluoromethyl)-
phenyl]benzamide;
3-{2-cyano-1-[4-(7H-pyrrolo[2,3-d]pyrimidin-4-yl)-1H-pyrazol-1-yl]ethyl}-N,N-dimethyl-
benzenesulfonamide;
3-{2-cyano-1-[4-(7H-pyrrolo[2,3-d]pyrimidin-4-yl)-1H-pyrazol-1-yl]ethyl}-N,N-dimethyl-
benzenesulfonamide;
N-benzyl-3-{2-cyano-1-[4-(7H-pyrrolo[2,3-d]pyrimidin-4-yl)-1H-pyrazol-1-yl]ethyl}-
benzenesulfonamide;
N-benzyl-3-{2-cyano-1-[4-(7H-pyrrolo[2,3-d]pyrimidin-4-yl)-1H-pyrazol-1-yl]ethyl}-
benzamide;
3-{2-cyano-1-[4-(7H-pyrrolo[2,3-d]pyrimidin-4-yl)-1H-pyrazol-1-yl]ethyl}-N-phenyl-
benzamide;
3-{2-cyano-1-[4-(7H-pyrrolo[2,3-d]pyrimidin-4-yl)-1H-pyrazol-1-yl]ethyl}-N-[3-
(trifluoro-methyl)phenyl]benzamide;
N-(3-cyanophenyl)-3-{[4-(7H-pyrrolo[2,3-d]pyrimidin-4-yl)-1H-pyrazol-1-yl]methyl}-
benzamide;
N-benzyl-3-{[4-(7H-pyrrolo-′[2,3-d]pyrimidin-4-yl)-1H-pyrazol-1-yl]methyl}benzamide;
N-1-naphthyl-3-{[4-(7H-pyrrolo[2,3-d]pyrimidin-4-yl)-1H-pyrazol-1-
yl]methyl}benzamide;
N-2-naphthyl-3-{[4-(7H-pyrrolo[2,3-d]-′pyrimidin-4-yl)-1H-pyrazol-1-
yl]methyl}benzamide;
N-(3-{[4-(7H-pyrrolo[2,3-d]pyrimidin-4-yl)-1H-pyrazol-1-yl]methyl}phenyl)-2-
naphthamide;
N-(3-{[4-(7H-pyrrolo[2,3-d]pyrimidin-4-yl)-1H-pyrazol-1-yl]methyl}phenyl)-1-
naphthamide;
2-phenyl-N-(3-{[4-(7H-pyrrolo[2,3-d]pyrimidin-4-yl)-1H-pyrazol-1-yl]methyl}phenyl)-
acetamide;
3-chloro-N-(3-{[4-(7H-pyrrolo[2,3-d]pyrimidin-4-yl)-1H-pyrazol-1-yl]methyl}phenyl)-
benzamide;
N-(3-{2-cyano-1-[4-(7H-pyrrolo[2,3-d]pyrimidin-4-yl)-1H-pyrazol-1-yl]ethyl}phenyl)-2-
naphthamide;
N-(3-{2-cyano-1-[4-(7H-pyrrolo[2,3-d]pyrimidin-4-yl)-1H-pyrazol-1-yl]ethyl}phenyl)-1-
naphthamide;
N-(3-{2-cyano-1-[4-(7H-pyrrolo[2,3-d]pyrimidin-4-yl)-1H-pyrazol-1-yl]-ethyl}phenyl)-
2-phenylacetamide;
3-cyano-N-(3-{2-cyano-1-[4-(7H-pyrrolo[2,3-d]pyrimidin-4-yl)-1H-pyrazol-1-
yl]ethyl}phenyl)benzamide;
N-(3-{2-cyano-1-[4-(7H-pyrrolo[2,3-d]pyrimidin-4-yl)-1H-pyrazol-1-yl]ethyl}phenyl)-
benzamide;
N-(3-{2-cyano-1-[4-(7H-pyrrolo[2,3-d]pyrimidin-4-yl)-1H-pyrazol-1-yl]ethyl}phenyl)-4-
(trifluoromethyl)benzamide;
N-(3-{2-cyano-1-[4-(7H-pyrrolo[2,3-d]pyrimidin-4-yl)-1H-pyrazol-1-yl]ethyl}phenyl)-
N′-phenylurea;
3-{2-cyano-1-[4-(7H-pyrrolo[2,3-d]pyrimidin-4-yl)-1H-pyrazol-1-yl]ethyl}-N-[4-
(trifluoromethyl)phenyl]benzamide;
3-{2-cyano-1-[4-(7H-pyrrolo[2,3-d]pyrimidin-4-yl)-1H-pyrazol-1-yl]ethyl}-N-(4-
methylphenyl)benzamide;
N-(4-cyanophenyl)-3-{2-cyano-1-[4-(7H-pyrrolo[2,3-d]pyrimidin-4-yl)-1H-pyrazol-1-
yl]-ethyl}benzamide;
3-{2-cyano-1-[4-(7H-pyrrolo[2,3-d]pyrimidin-4-yl)-1H-pyrazol-1-yl]ethyl}-N-2-
naphthylbenzamide;
3-{2-cyano-1-[4-(7H-pyrrolo[2,3-d]pyrimidin-4-yl)-1H-pyrazol-1-yl]ethyl}-N-1-
naphthylbenzamide;
3-{2-cyano-1-[4-(7H-pyrrolo[2,3-d]pyrimidin-4-yl)-1H-pyrazol-1-yl]ethyl}-N,N-
dimethylbenzamide;
3-{2-cyano-1-[4-(7H-pyrrolo[2,3-d]pyrimidin-4-yl)-1H-pyrazol-1-yl]ethyl}-N-pyridin-3-
ylbenzamide;
3-{2-cyano-1-[4-(7H-pyrrolo[2,3-d]pyrimidin-4-yl)-1H-pyrazol-1-yl]ethyl}-N-methyl-N-
phenylbenzamide;
3-{2-cyano-1-[4-(7H-pyrrolo[2,3-d]pyrimidin-4-yl)-1H-pyrazol-1-yl]ethyl}-N-
cyclohexylbenzamide;
3-{2-cyano-1-[4-(7H-pyrrolo[2,3-d]pyrimidin-4-yl)-1H-pyrazol-1-yl]ethyl}-N-(4-
phenoxyphenyl)benzamide;
N-(3-cyanophenyl)-3-{2-cyano-1-[4-(7H-pyrrolo[2,3-d]pyrimidin-4-yl)-1H-pyrazol-1-
yl]ethyl}benzamide;
N-biphenyl-4-yl-3-{2-cyano-1-[4-(7H-pyrrolo[2,3-d]pyrimidin-4-yl)-1H-pyrazol-1-
yl]ethyl}benzamide;
N-(4-chlorophenyl)-3-{2-cyano-1-[4-(7H-pyrrolo[2,3-d]pyrimidin-4-yl)-1H-pyrazol-1-
yl]-ethyl}benzamide;
3-{2-cyano-1-[4-(7H-pyrrolo[2,3-d]pyrimidin-4-yl)-1H-pyrazol-1-yl]ethyl}-N-(3,4-
dimethylphenyl)benzamide;
3-{2-cyano-1-[4-(7H-pyrrolo[2,3-d]pyrimidin-4-yl)-1H-pyrazol-1-yl]ethyl}-N-(3-
methoxyphenyl)benzamide;
3-{2-cyano-1-[4-(7H-pyrrolo[2,3-d]pyrimidin-4-yl)-1H-pyrazol-1-yl]ethyl}-N-(4-
methoxyphenyl)benzamide;
3-{2-cyano-1-[4-(7H-pyrrolo[2,3-d]pyrimidin-4-yl)-1H-pyrazol-1-yl]ethyl}-N-isoxazol-3-
yl-benzamide;
3-{2-cyano-1-[4-(7H-pyrrolo[2,3-d]pyrimidin-4-yl)-1H-pyrazol-1-yl]ethyl}-N-methyl-N-
phenylbenzenesulfonamide;
3-{2-cyano-1-[4-(7H-pyrrolo[2,3-d]pyrimidin-4-yl)-1H-pyrazol-1-yl]ethyl}-N-propyl-
benzenesulfonamide;
3-{2-cyano-1-[4-(7H-pyrrolo[2,3-d]pyrimidin-4-yl)-1H-pyrazol-1-yl]ethyl}-N-phenyl-
benzenesulfonamide;
3-{2-cyano-1-[4-(7H-pyrrolo[2,3-d]pyrimidin-4-yl)-1H-pyrazol-1-yl]ethyl}-N-2-
naphthyl-benzenesulfonamide;
3-{2-cyano-1-[4-(7H-pyrrolo[2,3-d]pyrimidin-4-yl)-1H-pyrazol-1-yl]ethyl}-N-
cyclopropyl-benzenesulfonamide;
3-[3-(piperidin-1-ylsulfonyl)phenyl]-3-[4-(7H-pyrrolo[2,3-d]pyrimidin-4-yl)-1H-
pyrazol-1-yl]propanenitrile;
3-[3-(morpholin-4-ylsulfonyl)phenyl]-3-[4-(7H-pyrrolo[2,3-d]pyrimidin-4-yl)-1H-
pyrazol-1-yl]propanenitrile;
3-{2-cyano-1-[4-(7H-pyrrolo[2,3-d]pyrimidin-4-yl)-1H-pyrazol-1-yl]ethyl}-N-(4-
methylphenyl)benzenesulfonamide;
3-{2-cyano-1-[4-(7H-pyrrolo[2,3-d]pyrimidin-4-yl)-1H-pyrazol-1-yl]ethyl}-N-(3,4-
dimethylphenyl)benzenesulfonamide;
3-{2-cyano-1-[4-(7H-pyrrolo[2,3-d]pyrimidin-4-yl)-1H-pyrazol-1-yl]ethyl}-N-(3-
methoxyphenyl)benzenesulfonamide;
3-{2-cyano-1-[4-(7H-pyrrolo[2,3-d]pyrimidin-4-yl)-1H-pyrazol-1-yl]ethyl}-N-(4-
methoxyphenyl)benzenesulfonamide;
3-{2-cyano-1-[4-(7H-pyrrolo[2,3-d]pyrimidin-4-yl)-1H-pyrazol-1-yl]ethyl}-N-(3,5-
dimethoxyphenyl)benzamide;
3-{2-cyano-1-[4-(7H-pyrrolo[2,3-d]pyrimidin-4-yl)-1H-pyrazol-1-yl]ethyl}-N-[4-
(dimethylamino)phenyl]benzamide;
3-[3-(benzylsulfonyl)phenyl]-3-[4-(7H-pyrrolo[2,3-d]pyrimidin-4-yl)-1H-pyrazol-1-
yl]propanenitrile;
3-[3-(benzylthio)phenyl]-3-[4-(7H-pyrrolo[2,3-d]pyrimidin-4-yl)-1H-pyrazol-1-yl]-
propanenitrile;
4-{[(3-{2-cyano-1-[4-(7H-pyrrolo[2,3-d]pyrimidin-4-yl)-1H-pyrazol-1-yl]ethyl}phenyl)-
sulfonyl]methyl}benzonitrile;
3-{2-cyano-1-[4-(7H-pyrrolo[2,3-d]pyrimidin-4-yl)-1H-pyrazol-1-yl]ethyl}-N-methyl-
benzenesulfonamide;
3-{2-cyano-1-[4-(7H-pyrrolo[2,3-d]pyrimidin-4-yl)-1H-pyrazol-1-yl]ethyl}-N-1-
naphthylbenzenesulfonamide;
N-biphenyl-4-yl-3-{2-cyano-1-[4-(7H-pyrrolo[2,3-d]pyrimidin-4-yl)-1H-pyrazol-1-
yl]ethyl}benzenesulfonamide;
3-{2-cyano-1-[4-(7H-pyrrolo[2,3-d]pyrimidin-4-yl)-1H-pyrazol-1-yl]ethyl}-N-[4-
(trifluoro-methoxy)phenyl]benzamide;
3-{2-cyano-1-[4-(7H-pyrrolo[2,3-d]pyrimidin-4-yl)-1H-pyrazol-1-yl]ethyl}-N-(2-
methoxy-phenyl)benzamide;
3-[3-(benzyloxy)phenyl]-3-[4-(7H-pyrrolo[2,3-d]pyrimidin-4-yl)-1H-pyrazol-1-
yl]propanenitrile;
3-{2-cyano-1-[4-(7H-pyrrolo[2,3-d]pyrimidin-4-yl)-1H-pyrazol-1-yl]ethyl}-N-
cyclohexylbenzenesulfonamide;
3-[3-(3,4-dihydroisoquinolin-2(1H)-ylsulfonyl)phenyl]-3-[4-(7H-pyrrolo[2,3-
d]pyrimidin-4-yl)-1H-pyrazol-1-yl]propanenitrile;
3-{2-cyano-1-[4-(7H-pyrrolo[2,3-d]pyrimidin-4-yl)-1H-pyrazol-1-yl]ethyl}-N-(2-
methoxy-ethyl)benzenesulfonamide;
3-{2-cyano-1-[4-(7H-pyrrolo[2,3-d]pyrimidin-4-yl)-1H-pyrazol-1-yl]ethyl}-N,N-diethyl-
benzenesulfonamide;
3-{3-[(4-ethylpiperazin-1-yl)sulfonyl]phenyl}-3-[4-(7H-pyrrolo[2,3-d]-pyrimidin-4-yl)-
1H-pyrazol-1-yl]propanenitrile;
N-1,3-benzodioxol-5-yl-3-{2-cyano-1-[4-(7H-pyrrolo[2,3-d]pyrimidin-4-yl)-1H-
pyrazol-1-yl]ethyl}benzenesulfonamide;
3-{3-[(3-methoxybenzyl)sulfonyl]phenyl}-3-[4-(7H-pyrrolo[2,3-d]pyrimidin-4-yl)-1H-
pyrazol-1-yl]propanenitrile;
3-{3-[(4-methoxybenzyl)sulfonyl]phenyl}-3-[4-(7H-pyrrolo[2,3-d]pyrimidin-4-yl)-1H-
pyrazol-1-yl]propanenitrile;
3-{3-[(2,6-dimethylmorpholin-4-yl)sulfonyl]phenyl}-3-[4-(7H-pyrrolo[2,3-d]pyrimidin-
4-yl)-1H-pyrazol-1-yl]propanenitrile;
3-{3-[(4-oxopiperidin-1-yl)sulfonyl]phenyl}-3-[4-(7H-pyrrolo[2,3-d]pyrimidin-4-yl)-1H-
pyrazol-1-yl]propanenitrile;
3-[3-(isopropylsulfonyl)phenyl]-3-[4-(7H-pyrrolo[2,3-d]pyrimidin-4-yl)-1H-pyrazol-1-
yl]propanenitrile;
3-{3-[(cyclohexylmethyl)sulfonyl]phenyl}-3-[4-(7H-pyrrolo[2,3-d]pyrimidin-4-yl)-1H-
pyrazol-1-yl]propanenitrile;
3-[3-(octahydroisoquinolin-2(1H)-ylsulfonyl)phenyl]-3-[4-(7H-pyrrolo[2,3-d]pyrimidin-
4-yl)-1H-pyrazol-1-yl]propanenitrile;
3-{3-[(2-phenylethyl)-sulfonyl]phenyl}-3-[4-(7H-pyrrolo[2,3-d]pyrimidin-4-yl)-1H-
pyrazol-1-yl]propanenitrile;
cis-4-[4-(7H-pyrrolo[2,3-d]pyrimidin-4-yl)-1H-pyrazol-1-yl]cyclohexylacetonitrile;
cis-4-[4-(7H-pyrrolo[2,3-d]pyrimidin-4-yl)-1H-pyrazol-1-
yl]cyclohexylmethylthiocyanate;
N-5-[(cis-4-[4-(7H-pyrrolo[2,3-d]pyrimidin-4-yl)-1H-pyrazol-1-
yl]cyclohexylmethyl)thio]-4H-1,2,4-triazol-3-ylpyrimidin-2-amine;
N-5-[(cis-4-[4-(7H-pyrrolo[2,3-d]pyrimidin-4-yl)-1H-pyrazol-1-
yl]cyclohexylmethyl)thio]-4H-1,2,4-triazol-3-ylpyrimidin-2-amine;
3-cis-4-[4-(7H-pyrrolo[2,3-d]pyrimidin-4-yl)-1H-pyrazol-1-yl]cyclohexylpropanenitrile;
5-[(2-cis-4-[4-(7H-pyrrolo[2,3-d]pyrimidin-4-yl)-1H-pyrazol-1-yl]cyclohexylethyl)thio]-
4H-1,2,4-triazol-3-amine;
4-[4-(7H-pyrrolo[2,3-d]pyrimidin-4-yl)-1H-pyrazol-1-yl]cyclohexylideneacetonitrile;
cis-4-[4-(7H-pyrrolo[2,3-d]pyrimidin-4-yl)-1H-pyrazol-1-yl]cyclohexanecarbonitrile;
2-[(cis-4-[4-(7H-pyrrolo[2,3-d]pyrimidin-4-yl)-1H-pyrazol-1-
yl]cyclohexylmethyl)sulfinyl]-benzonitrile;
2-[(cis-4-[4-(7H-pyrrolo[2,3-d]pyrimidin-4-yl)-1H-pyrazol-1-
yl]cyclohexylmethyl)sulfonyl]-benzonitrile;
3-[4-(7H-pyrrolo[2,3-d]pyrimidin-4-yl)-1H-pyrazol-1-yl]cyclohexylacetonitrile;
5-({cis-4-[4-(7H-pyrrolo[2,3-d]pyrimidin-4-yl)-1H-pyrazol-1-yl]cyclohexyl}thio)-1H-
1,2,4-triazol-3-amine;
N-{5-[({cis-4-[4-(7H-pyrrolo[2,3-d]pyrimidin-4-yl)-1H-pyrazol-1-yl]cyclohexyl}methyl)-
thio]-4H-1,2,4-triazol-3-yl}methanesulfonamide;
[cis-4-[4-(7H-Pyrrolo[2,3-d]pyrimidin-4-yl)-1H-pyrazol-1-yl]-1-(1H-1,2,4-triazol-1-
yl)cyclohexyl]acetonitrile;
3-[3-(piperazin-1-ylsulfonyl)phenyl]-3-[4-(7H-pyrrolo[2,3-d]pyrimidin-4-yl)-1H-
pyrazol-1-yl]propanenitrile;
3-[4-(7H-pyrrolo[2,3-d]pyrimidin-4-yl)-1H-pyrazol-1-yl]-3-[3-(thiomorpholin-4-
ylsulfonyl)-phenyljpropanenitrile;
3-{3-[(4-hydroxypiperidin-1-yl)sulfonyl]phenyl}-3-[4-(7H-pyrrolo[2,3-d]pyrimidin-4-
yl)-1H-pyrazol-1-yl]propanenitrile;
3-[3-(isobutylsulfonyl)phenyl]-3-[4-(7H-pyrrolo[2,3-d]pyrimidin-4-yl)-1H-pyrazol-1-
yl]propanenitrile;
3-[4-(7H-pyrrolo[2,3-d]pyrimidin-4-yl)-1H-pyrazol-1-yl]-3-{3-[(tetrahydro-2H-pyran-4-
ylmethyl)sulfonyl]phenyl}propanenitrile;
3-{3-[(2-methoxyethyl)sulfonyl]phenyl}-3-[4-(7H-pyrrolo[2,3-d]pyrimidin-4-yl)-1H-
pyrazol-1-yl]propanenitrile;
3-{3-[(3-furylmethyl)sulfonyl]phenyl}-3-[4-(7H-pyrrolo[2,3-d]pyrimidin-4-yl)-1H-
pyrazol-1-yl]propanenitrile;
3-{3-[(1,1-dioxidothiomorpholin-4-yl)sulfonyl]phenyl}-3-[4-(7H-pyrrolo[2,3-
d]pyrimidin-4-yl)-1H-pyrazol-1-yl]propanenitrile;
3-{3-[(4-acetylpiperazin-1-yl)sulfonyl]phenyl}-3-[4-(7H-pyrrolo[2,3-d]pyrimidin-4-yl)-
IH-pyrazol-1-yl]propanenitrile;
3-{3-[(pyridin-4-ylmethyl)sulfonyl]phenyl}-3-[4-(7H-pyrrolo[2,3-d]pyrimidin-4-yl)-1H-
pyrazol-1-yl]propanenitrile;
4-[1-(1-phenylbut-3-yn-1-yl)-1H-pyrazol-4-yl]-7H-pyrrolo[2,3-d]pyrimidine;
4-(1-{1-[3-(morpholin-4-ylsulfonyl)phenyl]but-3-yn-1-yl}-1H-pyrazol-4-yl)-7H-
pyrrolo[2,3-d]pyrimidine;
3-{1-[4-(7H-pyrrolo[2,3-d]pyrimidin-4-yl)-1H-pyrazol-1-yl]but-3-yn-1-yl]benzonitrile;
3-{1-[4-(7H-pyrrolo[2,3-d]-pyrimidin-4-yl)-1H-pyrazol-1-yl]but-3-yn-1-
yl]benzaldehyde;
methyl3-(3-cyanophenyl)-3-[4-(7H-pyrrolo[2,3-d]pyrimidin-4-yl)-1H-pyrazol-1-yl]-
propanoate;
N,N-dimethyl-3-{1-[4-(7H-pyrrolo[2,3-d]pyrimidin-4-yl)-1H-pyrazol-1-yl]but-3-yn-1-
yl}-benzenesulfonamide;
3-{2-cyano-1-[4-(7H-pyrrolo[2,3-d]pyrimidin-4-yl)-1H-pyrazol-1-yl]ethyl}-N-[4-
(dimethy-amino)phenyl]benzenesulfonamide;
3-{3-methoxy-1-[4-(7H-pyrrolo[2,3-d]pyrimidin-4-yl)-1H-pyrazol-1-yl]propyl}-N,N-
dimethylbenzenesulfonamide;
N-phenyl-3-{1-[4-(7H-pyrrolo[2,3-d]pyrimidin-4-yl)-1H-pyrazol-1-yl]but-3-yn-1-yl}-
benzamide;
4-[1-(3-methoxy-1-phenylpropyl)-1H-pyrazol-4-yl]-7H-pyrrolo[2,3-d]pyrimidine;
N-[4-(dimethylamino)phenyl]-3-{1-[4-(7H-pyrrolo[2,3-d]-pyrimidin-4-yl)-1H-pyrazol-1-
yl]but-3-yn-1-yl}benzamide;
3-{3-hydroxy-1-[4-(7H-pyrrolo[2,3-d]pyrimidin-4-yl)-1H-pyrazol-1-yl]propyl}-N,N-
dimethylbenzenesulfonamide;
3-{1-[4-(7H-pyrrolo[2,3-d]pyrimidin-4-yl)-1H-pyrazol-1-yl]but-3-en-1-yl}benzonitrile;
4-{1-[I-(3-bromophenyl)but-3-en-1-yl]-1H-pyrazol-4-yl}-7H-pyrrolo[2,3-d]pyrimidine;
3-{4,4-difluoro-1-[4-(7H-pyrrolo[2,3-d]pyrimidin-4-yl)-1H-pyrazol-1-yl]but-3-en-1-yl}-
benzonitrile;
4-(1-{4,4-difluoro-1-[3-(morpholin-4-ylsulfonyl)phenyl]but-3-en-1-yl}-1H-pyrazol-4-
yl)-7H-pyrrolo[2,3-d]pyrimidine;
4-(1-{1-[3-(ethylsulfonyl)phenyl]-4,4-difluorobut-3-en-1-yl}-1H-pyrazol-4-yl)-7H-
pyrrolo-[2,3-d]pyrimidine;
4-(1-{1[3-(benzyloxy)phenyl]-4,4-difluorobut-3-en-1-yl}-1H-pyrazol-4-yl)-7H-
pyrrolo[2,3-d]pyrimidine;
4-[1-(2-methoxy-1-phenylethyl)-1H-pyrazol-4-yl]-7H-pyrrolo[2,3-d]pyrimidine;
4-(1-{4ⱼ4-difluoro-1[3-(methylsulfonyl)phenyl]but-3-en-1-yl}-1H-pyrazol-4-yl)-7H-
pyrrolo[2,3-d]pyrimidine;
3-{[4-(7H-pyrrolo[2,3-d]pyrimidin-4-yl)-1H-pyrazol-1-yl]methyl}benzonitrile;
3-{1-[4-(7H-pyrrolo[2,3-d]pyrimidin-4-yl)-1H-pyrazol-1-yl]-′butyl]benzonitrile;
4-(1-{1-[3-(ethylsulfonyl)phenyl]-4,4-difluorobutyl}-1H-pyrazol-4-yl)-7H-pyrrolo[2,3-d]-
pyrimidine;
4-[1-(4,4-difluoro-1-{3-[(2-methoxyethyl)sulfonyl]phenyl}-but-3-en-1-yl)-1H-pyrazol-4-
yl]-7H-pyrrolo[2,3-d]pyrimidine;
4-[1-(1-cyclopentylbut-3-en-1-yl)-1H-pyrazol-4-yl]-7H-pyrrolo[2,3-d]pyrimidine;
4-[1-(1-methylbut-3-en-1-yl)-1H-pyrazol-4-yl]-7H-pyrrolo[2,3-d]pyrimidine;
4-[1-(1-cyclopentyl-2-cyclopropylethyl)-1H-pyrazol-4-yl]-7H-pyrrolo[2,3-d]pyrimidine;
4-[1-(1-cyclopentylbut-3-yn-1-yl)-1H-pyrazol-4-yl]-7H-pyrrolo[2,3-d]pyrimidine;
4-[1-(1-cyclopentylbutyl)-1H-pyrazol-4-yl]-7H-pyrrolo[2,3-d]pyrimidine;
4-[1-(1-cyclopentyl-4,4-difluorobut-3-en-1-yl)-1H-pyrazol-4-yl]-7H-pyrrolo[2,3-d]-
pyrimidine;
4-{1-[4,4-difluoro-1-(tetrahydrofuran-3-yl)but-3-en-1-yl]-1H-pyrazol-4-yl}-7H-
pyrrolo[2,3-d]pyrimidine;
4-(1-(5,5-difluoropent-4-en-2-yl)-1H-pyrazol-4-yl)-7H-pyrrolo[2,3-d]pyrimidine;
4-[1-(1-cyclopropyl-4,4-difluorobut-3-en-1-yl)-1H-pyrazol-4-yl]-7H-pyrrolo[2,3-
d]pyrimidine;
4-[1-(1-cyclopentyl-4,4-difluorobutyl)-1H-pyrazol-4-yl]-7H-pyrrolo[2,3-d]pyrimidine;
3-(1-methylcyclopentyl)-3-[4-(7H-pyrrolo[2,3-d]pyrimidin-4-yl)-1H-pyrazol-1-
yl]propanenitrile;
4,4-dimethyl-3-[4-(7H-pyrrolo[2,3-d]pyrimidin-4-yl)-1H-pyrazol-1-yl]pentanenitrile;
1-{2-cyano-1-[4-(7H-pyrrolo[2,3-d]pyrimidin-4-yl)-1H-pyrazol-1-
yl]ethyl}cyclopropane-carbonitrile;
N-[(1-{2-cyano-1-[4-(7H-pyrrolo[2,3-d]pyrimidin-4-yl)-1H-pyrazol-1-
yl]ethyl}cyclopentyl)-methyl]benzamide;
3-{1-[(benzyloxy)methyl]cyclopentyl}-3-[4-(7H-pyrrolo[2,3-d]pyrimidin-4-yl)-1H-
pyrazol-1-yl]propanenitrile;
3-[1-(methylsulfonyl)pyrrolidin-3-yl]-3-[4-(7H-pyrrolo[2,3-d]pyrimidin-4-yl)-1H-pyrazol-
1-yl]propanenitrile;
N′-cyano-4-(cyanomethyl)-4-[4-(7H-pyrrolo[2,3-d]pyrimidin-4-yl)-1H-pyrazol-1-yl]-
piperidine-1-carboximidamide;
4-{1-[2,2,2-trifluoro-1-(1H-imidazol-2-ylmethyl)ethyl]-1H-pyrazol-4-yl}-7H-pyrrolo[2,3-
d]pyrimidine;
4-(1-2,2,2-trifluoro-1-[(4-methyl-1,3-thiazol-2-yl)methyl]ethyl-1H-pyrazol-4-yl)-
7H-pyrrolo[2,3-d]pyrimidine;
4-{1-[I-(trifluoromethyl)but-3-yn-1-yl]-1H-pyrazol-4-yl}-7H-pyrrolo[2,3-d]pyrimidine;
4-{1-[I-(trifluoromethyl)but-3-en-1-yl]-1H-pyrazol-4-yl}-7H-pyrrolo[2,3-d]pyrimidine;
4-{1-[1-(trifluoromethyl)butyl]-1H-pyrazol-4-yl}-7H-pyrrolo[2,3-d]pyrimidine;
4-{1-[4,4-difluoro-1-(trifluoromethyl)but-3-en-1-yl]-1H-pyrazol-4-yl}-7H-pyrrolo[2,3-
d]-pyrimidine;
4-{1-[4,4-difluoro-1-(trifluoromethyl)butyl]-1H-pyrazol-4-yl}-7H-pyrrolo[2,3-
d]pyrimidine;
3-pyridin-4-yl-3-[5-(7H-pyrrolo[2,3-d]pyrimidin-4-yl)-1,3-thiazol-2-yl]propanenitrile;
4-{2-cyano-1-[5-(7H-pyrrolo[2,3-d]pyrimidin-4-yl)-1,3-thiazol-2-yl]ethyl}pyridine-2-
carbonitrile;
3-pyridin-2-yl-3-[5-(7H-pyrrolo[2,3-d]pyrimidin-4-yl)-1,3-thiazol-2-yl]propanenitrile;
{1-(ethylsulfonyl)-3-[4-(7H-pyrrolo[2,3-d]pyrimidin-4-yl)-1H-pyrazol-1-yl]azetidin-3-
yl]acetonitrile;
1-(cyclopropylsulfonyl)-3-[4-(7H-pyrrolo[2,3-d]pyrimidin-4-yl)-1H-pyrazol-1-yl]azetidin-
3-ylacetonitrile;
1-[(1-methylcyclopropylcarbonyl)-3-[4-(7H-pyrrolo[2,3-d]pyrimidin-4-yl)-1H-pyrazol-
1-yl]azetidin-3-ylacetonitrile;
1-[(1-methylcyclopropyl)sulfonyl]-3-[4-(7H-pyrrolo[2,3-d]pyrimidin-4-yl)-1H-pyrazol-
1-yl]azetidin-3-ylacetonitrile;
1-(methylsulfonyl)-3-[4-(7H-pyrrolo[2,3-d]pyrimidin-4-yl)-1H-pyrazol-1-yl]azetidin-3-
yl]acetonitrile;
{1-(phenylsulfonyl)-3-[4-(7H-pyrrolo[2,3-d]pyrimidin-4-yl)-1H-pyrazol-1-yl]azetidin-3-
yl]acetonitrile;
{1-(isopropylsulfonyl)-3-[4-(7H-pyrrolo[2,3-d]pyrimidin-4-yl)-1H-pyrazol-1-yl]azetidin-
3-yl]acetonitrile;
{1-(propylsulfonyl)-3-[4-(7H-pyrrolo[2,3-d]pyrimidin-4-yl)-1H-pyrazol-1-yl]azetidin-3-
yl]acetonitrile;
{1-(butylsulfonyl)-3-[4-(7H-pyrrolo[2,3-d]pyrimidin-4-yl)-1H-pyrazol-1-yl]azetidin-3-yl}acetonitrile;
{1-(tert-butylsulfonyl)-3-[4-(7H-pyrrolo[2,3-d]pyrimidin-4-yl)-1H-pyrazol-1-yl]azetidin-
3-yl}acetonitrile;
3-(cyanomethyl)-N,N-dimethyl-3-[4-(7H-pyrrolo[2,3-d]pyrimidin-4-yl)-1H-pyrazol-1-
yl]azetidine-1-sulfonamide;
{1-[(1-methyl-1H-pyrazol-3-yl)sulfonyl]-3-[4-(7H-pyrrolo[2,3-d]pyrimidin-4-yl)-1H-
pyrazol-1-yl]azetidin-3-yl}acetonitrile;
{3-[4-(7H-pyrrolo[2,3-d]pyrimidin-4-yl)-1H-pyrazol-1-yl]-1-[(3,3,3-
trifluoropropyl)sulfonyl]azetidin-3-yl}acetonitrile;
{1-(isobutylsulfonyl)-3-[4-(7H-pyrrolo[2,3-d]pyrimidin-4-yl)-1H-pyrazol-1-yl]azetidin-3-
yl}acetonitrile;
{1-(sec-butylsulfonyl)-3-[4-(7H-pyrrolo[2,3-d]pyrimidin-4-yl)-1H-pyrazol-1-yl]azetidin-3-
yl}acetonitrile;
{1-[(5-methyl-2-thienyl)sulfonyl]-3-[4-(7H-pyrrolo[2,3-d]pyrimidin-4-yl)-1H-pyrazol-1-
yl]azetidin-3-yl}acetonitrile;
{1-[(4-fluorophenyl)sulfonyl]-3-[4-(7H-pyrrolo[2,3-d]pyrimidin-4-yl)-1H-pyrazol-1-
yl]azetidin-3-yl}acetonitrile;
{1-[(3-fluorophenyl)sulfonyl]-3-[4-(7H-pyrrolo[2,3-d]pyrimidin-4-yl)-1H-pyrazol-1-
yl]azetidin-3-yl}acetonitrile;
{1-[(2-fluorophenyl)sulfonyl]-3-[4-(7H-pyrrolo[2,3-d]pyrimidin-4-yl)-1H-pyrazol-1-
yl]azetidin-3-yl}acetonitrile;
{1-(pyridin-3-ylsulfonyl)-3[4-(7H-pyrrolo[2,3-d]pyrimidin-4-yl)-1H-pyrazol-1-yl]azetidin-
3-yl}acetonitrile;
{1-(pyridin-2-ylsulfonyl)-3-[4-(7H-pyrrolo[2,3-d]pyrimidin-4-yl)-1H-pyrazol-1-yl]azetidin-
3-yl}acetonitrile;
{1-(cyclopropylcarbonyl)-3-[4-(7H-pyrrolo[2,3-d]pyrimidin-4-yl)-1H-pyrazol-1-
yl]azetidin-3-yl}acetonitrile;
1-[(1-methylcyclopropyl)carbonyl-3-[4-(7H-pyrrolo[2,3-d]pyrimidin-4-yl)-1H-pyrazol-1-
yl]azetidin-3-ylacetonitrile;
{1-benzoyl-3-[4-(7H-pyrrolo[2,3-d]pyrimidin-4-yl)-1H-pyrazol-1-yl}azetidin-3-yl}acetonitrile;
{1-[(6-methylpyridin-2-yl)carbonyl]-3-[4-(7H-pyrrolo[2,3-d]pyrimidin-4-yl)-1H-
pyrazol-1-yl]azetidin-3-yl}acetonitrile;
{1-(pyridin-3-ylcarbonyl)-3-[4-(7H-pyrrolo[2,3-d]pyrimidin-4-yl)-1H-pyrazol-1-
yl]azetidin-3-yl}acetonitrile;
{1-(3-methylbenzoyl)-3-[4-(7H-pyrrolo[2,3-d]pyrimidin-4-yl)-1H-pyrazol-1-yl]azetidin-
3-yl}acetonitrile;
{1-(4-methylbenzoyl)-3-[4-(7H-pyrrolo[2,3-d]pyrimidin-4-yl)-1H-pyrazol-1-yl]azetidin-
3-yl}acetonitrile;
3-({3-(cyanomethyl)-3-[4-(7H-pyrrolo[2,3-d]pyrimidin-4-yl)-1H-pyrazol-1-yl]azetidin-1-
yl}carbonyl)benzonitrile;
[3-[4-(7H-pyrrolo[2,3-d]pyrimidin-4-yl)-1H-pyrazol-1-yl]-1-(2-thienylcarbonyl)azetidin-
3-yl]acetonitrile;
[3-[4-(7H-pyrrolo[2,3-d]pyrimidin-4-yl)-1H-pyrazol-1-yl]-1-(1H-pyrrol-2-
ylcarbonyl)azetidin-3-yl]acetonitrile;
{1-(1H-indol-2-ylcarbonyl)-3-[4-(7H-pyrrolo[2,3-d]pyrimidin-4-yl)-1H-pyrazol-1-
yl]azetidin-3-yl}acetonitrile;
{1-(isoxazol-5-ylcarbonyl)-3-[4-(7H-pyrrolo[2,3-d]pyrimidin-4-yl)-1H-pyrazol-1-
yl]azetidin-3-yl}acetonitrile;
{1-(1H-pyrazol-3-ylcarbonyl)-3-[4-(7H-pyrrolo[2,3-d]pyrimidin-4-yl)-1H-pyrazol-1-
yl]azetidin-3-yl}acetonitrile;
isobutyl 3-(cyanomethyl)-3-[4-(7H-pyrrolo[2,3-d]pyrimidin-4-yl)-1H-pyrazol-1-
yl]azetidine-1-carboxylate;
phenyl 3-(cyanomethyl)-3-[4-(7H-pyrrolo[2,3-d]pyrimidin-4-yl)-1H-pyrazol-1-
yl]azetidine-1-carboxylate;
benzyl 3-(cyanomethyl)-3-[4-(7H-pyrrolo[2,3-d]pyrimidin-4-yl)-1H-pyrazol-1-
yl]azetidine-1-carboxylate;
3-(cyanomethyl)-N-phenyl-3-[4-(7H-pyrrolo[2,3-d]pyrimidin-4-yl)-1H-pyrazol-1-
yl]azetidine-1-carboxamide;
3-(cyanomethyl)-N,N-dimethyl-3-[4-(7H-pyrrolo[2,3-d]pyrimidin-4-yl)-1H-pyrazol-1-yl]cyclobutanesulfonamide;
3-isoxazol-3-yl-1-[4-(7H-pyrrolo[2,3-d]pyrimidin-4-yl)-1H-pyrazol-1-yl]cyclobutylacetonitrile;
{3-(3-methyl-1,2,4-oxadiazol-5-yl)-1-[4-(7H-pyrrolo[2,3-d]pyrimidn-4-yl)-1H-pyrazol-
1-yl]cyclobutyl}acetonitrile;
{3-(3-tert-butyl-1,2,4-oxadiazol-5-yl)-1-[4-(7H-pyrrolo[2,3-d]pyrimidin-4-yl)-1H-
pyrazol-1-yl]cyclobutyl}acetonitrile;
1-[4-(7H-pyrrolo[2,3-d]pyrimidin-4-yl)-1H-pyrazol-1-yl]cyclobutylacetonitrile;
3-(hydroxymethyl)-1-[4-(7H-pyrrolo[2,3-d]pyrimidin-4-yl)-1H-pyrazol-1-yl]cyclobutylacetonitrile;
3-(fluoromethyl)-1-[4-(7H-pyrrolo[2,3-d]pyrimidin-4-yl)-1H-pyrazol-1-yl]cyclobutylacetonitrile;
3-(difluoromethyl)-1-[4-(7H-pyrrolo[2,3-d]pyrimidin-4-yl)-1H-pyrazol-1-yl]cyclobutylacetonitrile;
2,2′-[1-[4-(7H-pyrrolo[2,3-d]pyrimidin-4-yl)-1H-pyrazol-1-yl]cyclobutane-1,3-diyl]diacetonitrile;
3-(cyanomethyl)-1-methyl-3-[4-(7H-pyrrolo[2,3-d]pyrimidin-4-yl)-1H-pyrazol-1-
yl]cyclobutanecarbonitrile;
3-(cyanomethyl)-1-(methoxymethyl)-3-[4-(7H-pyrrolo[2,3-d]pyrimidin-4-yl)-1H-
pyrazol-1-yl]cyclobutanecarbonitrile;
3-(cyanomethyl)-1-(fluoromethyl)-3-[4-(7H-pyrrolo[2,3-d]pyrimidin-4-yl)-1H-pyrazol-
1-yl]cyclobutanecarbonitrile;
1,3-bis(cyanomethyl)-3-[4-(7H-pyrrolo[2,3-d]pyrimidin-4-yl)-1H-pyrazol-1-
yl]cyclobutanecarbonitrile;
3-(cyanomethyl)-3-[4-(7H-pyrrolo[2,3-d]pyrimidin-4-yl)-1H-pyrazol-1-
yl]cyclobutanecarbonitrile;
3,3-bis(hydroxymethyl)-1-[4-(7H-pyrrolo[2,3-d]pyrimidin-4-yl)-1H-pyrazol-1-
yl]cyclobutylacetonitrile;
3,3-bis(fluoromethyl)-1-[4-(7-[2-(trimethylsilyl)ethoxy]methyl-7H-pyrrolo[2,3-
d]pyrimidin-4-yl)-1H-pyrazol-1-yl]cyclobutylacetonitrile;
2,2′,2″-[1-[4-(7H-pyrrolo[2,3-d]pyrimidin-4-yl)-1H-pyrazol-1-yl]cyclobutane-1,3,3-
triyl]triacetonitrile;
3-hydroxy-1-[4-(7H-pyrrolo[2,3-d]pyrimidin-4-yl)-1H-pyrazol-1-yl]cyclobutylacetonitrile;
3-fluoro-1-[4-(7H-pyrrolo[2,3-d]pyrimidin-4-yl)-1H-pyrazol-1-yl]cyclobutylacetonitrile;
3-methyl-1-[4-(7H-pyrrolo[2,3-d]pyrimidin-4-yl)-1H-pyrazol-1-yl]cyclobutylacetonitrile;
3,3-dimethyl-1-[4-(7H-pyrrolo[2,3-d]pyrimidin-4-yl)-1H-pyrazol-1-
yl]cyclobutylacetonitrile;
3-(benzyloxy)-1-[4-(7H-pyrrolo[2,3-d]pyrimidin-4-yl)-1H-pyrazol-1-
yl]cyclobutylacetonitrile;
3-(cyanomethyl)-3-[4-(7H-pyrrolo[2,3-d]pyrimidin-4-yl)-1H-pyrazol-1-
yl]cyclobutanecarbonitrile;
{1-(Ethylsulfonyl)-3-[4-(7H-pyrrolo[2,3-d]pyrimidin-4-yl)-1H-pyrazol-1-yl]azetidin-3-
yl}acetonitrile
9-Fluoro-2-piperidin-1-ylbenzo[h][1,3]thiazolo[5,4-f]isoquinolin-7(6H)-one;
2-(tert-Butylamino)-9-fluorobenzo[h][1,3]thiazolo[5,4-f]isoquinolin-7(6H)-one;
9-Fluoro-2-[(3-methoxypropyl)amino]benzo[h][1,3]thiazolo[5,4-f]isoquinolin-7(6H)-one;
9-Fluoro-2-(4-methylpiperazin-1-yl)benzo[h][1,3]thiazolo[5,4-f]isoquinolin-7(6H)-one;
2-(Dimethylamino)-9-fluorobenzo[h][1,3]thiazolo[5,4-f]isoquinolin-7(6H)-one;
2-(Benzylamino)-9-fluorobenzo[h][1,3]thiazolo[5,4-f]isoquinolin-7(6H)-one;
2-Anilino-9-fluorobenzo[h][1,3]thiazolo[5,4-f]isoquinolin-7(6H)-one;
9-Fluoro-2,6-dihydro-7H-benzo[h]pyrazolo[4,3-f]isoquinolin-7-one;
9-Fluoro-2-piperidin-1-ylbenzo[h][1,3]oxazolo[5,4-f]isoquinolin-7(6H)-one;
2-tert-Butyl-9-fluoro-3H-benzo[h]imidazo[4,5-f]quinoline;
2-tert-Butyl-9-fluoro-3H-benzo[h]imidazo[4,5-f]quinoline7-oxide;
2-tert-Butyl-9-fluoro-7-methoxy-3H-benzo[f]imidazo[4,5-h]phthalazin-3-ol
trifluoroacetic acid;
2-tert-Butyl-9-fluoro-7-methoxy-3H-1,3,5,6-tetraaza-cyclopenta[l]phenanthrene;
2-tert-Butyl-9-fluoro-3,6-dihydro-1,3,5,6-tetraaza-cyclopenta[l]phenanthren-7-one;
2-tert-Butyl-9-fluoro-3H-benzo[f]imidazo[4,5-h]phthalazine-3,7-diol;
2-tert-Butyl-9-fluoro-7-methoxy-3H-benzo[f]imidazo[4,5-h]quinazolin-5-amine;
5-Amino-2-tert-butyl-9-fluoro-3H-benzo[f]imidazo[4,5-h]quinazolin-7-ol;
2-tert-Butyl-9-fluoro-7-methoxy-3H-benzo[f]imidazo[4,5-h]quinazoline;
2-tert-Butyl-9-fluoro-3H-benzo[f]imidazo[4,5-h]quinazolin-7-ol;
2-tert-Butyl-9-fluoro-3H-benzo[h]imidazo[4,5-f]isoquinolin-3-ol;
2-tert-Butyl-9-fluoro-3H-benzo[h]imidazo[4,5-f]isoquinoline;
2-tert-Butyl-9-fluoro-3H-benzo[h]imidazo[4,5-f]isoquinoline6-oxide;
9-Fluoro-2-[hydroxy(pyridin-3-yl)methyl]benzo[h][1,3]thiazolo[5,4-f]isoquinolin-7(6H)-
one;
9-Fluoro-2-[4-(1H-imidazol-4-ylcarbonyl)piperazin-1-yl]benzo[h][1,3]thiazolo[5,4-
f]isoquinolin-7(6H)-one;
2-Ethyl-9-fluoro-3,6-dihydro-7H-benzo[f]imidazo[4,5-h]quinazolin-7-one;
trans-2-(9-fluoro-7-oxo-6,7-dihydro-3H-benzo[f]imidazo[4,5-h]quinazolin-2-
yl)cyclopropanecarboxamide;
1-(9-fluoro-7-oxo-6,7-dihydro-3H-benzo[f]imidazo[4,5-h]quinazolin-2-
yl)cyclopropanecarboxylic acid;
2-[2-(dimethylamino)-1,1-dimethylethyl]-9-fluoro-3,6-dihydro-7H-benzo[f]imidazo[4,5-
h]quinazolin-7-one;
4-ethyl-4-(9-fluoro-7-oxo-6,7-dihydro-3H-benzo[f]imidazo[4,5-h]quinazolin-2-
yl)hexanenitrile;
4-ethyl-4-(9-fluoro-7-oxo-6,7-dihydro-3H-benzo[f]imidazo[4,5-h]quinazolin-2-
yl)hexanamide;
2-(4-amino-1,1-dimethylbutyl)-9-fluoro-3,6-dihydro-7H-benzo[f]imidazo[4,5-
h]quinazolin-7-one;
benzyl[1-(9-fluoro-7-oxo-6,7-dihydro-3H-benzo[f]imidazo[4,5-h]quinazolin-2-yl)-1-
methylethyl]carbamate;
benzyl[2-(9-fluoro-7-oxo-6,7-dihydro-3H-benzo[f]imidazo[4,5-h]quinazolin-2-yl)-2-
methylpropyl]carbamate;
[2-(9-fluoro-7-oxo-6,7-dihydro-3H-benzo[f]imidazo[4,5-h]quinazolin-2-yl)-2-
methylpropoxy]acetonitrile;
2-(1-amino-1-methylethyl)-9-fluoro-3,6-dihydro-7H-benzo[f]imidazo[4,5-h]quinazolin-7-
one;
4-(9-fluoro-7-oxo-6,7-dihydro-3H-benzo[f]imidazo[4,5-h]quinazolin-2-yl)butanenitrile;
N-[1-(9-fluoro-7-oxo-6,7-dihydro-3H-benzo[f]imidazo[4,5-h]quinazolin-2-yl)-1-methylethyl]acetamide;
benzyl4-(9-fluoro-7-oxo-6,7-dihydro-3H-benzo[f]imidazo[4,5-h]quinazolin-2-
yl)piperidine-1-carboxylate;
3-(9-fluoro-7-oxo-6,7-dihydro-3H-benzo[f]imidazo[4,5-h]quinazolin-2-yl)propanenitrile;
N-[2-(9-fluoro-7-oxo-6,7-dihydro-3H-benzo[f]imidazo[4,5-h]quinazolin-2-yl)-2-
methylpropyl]urea;
4-(9-fluoro-7-oxo-6,7-dihydro-3H-benzo[f]imidazo[4,5-h]quinazolin-2-yl)-4-
methylpentanenitrile;
2-(1-acetylpiperidin-4-yl)-9-fluoro-3,6-dihydro-7H-benzo[f]imidazo[4,5-h]quinazolin-7-
one;
9-fluoro-2-(trans-4-hydroxycyclohexyl)-3,6-dihydro-7H-benzo[f]imidazo[4,5-
h]quinazolin-7-one;
9-fluoro-2-(cis-4-hydroxycyclohexyl)-3,6-dihydro-7H-benzo[f]imidazo[4,5-
h]quinazolin-7-one;
3-(9-fluoro-7-oxo-6,7-dihydro-3H-benzo[f]imidazo[4,5-h]quinazolin-2-yl)-3-
methylbutanenitrile;
2-(Ethylthio)-9-fluoro-3,6-dihydro-7H-benzo[h]imidazo[4,5-f]isoquinolin-7-one;
2-(Ethylsulfinyl)-9-fluoro-3,6-dihydro-7H-benzo[h]imidazo[4,5-f]isoquinolin-7-one;
2-(Ethylsulfonyl)-9-fluoro-3,6-dihydro-7H-benzo[h]imidazo[4,5-f]isoquinolin-7-one;
2-[(9-Fluoro-7-oxo-6,7-dihydro-3H-benzo[h]imidazo[4,5-f]isoquinolin-2-yl)thio]-4-
hydroxybutanamide;
9-Fluoro-2-[(2-hydroxycyclohexyl)thio]-3,6-dihydro-7H-benzo[h]imidazo[4,5-
f]isoquinolin-7-one;
2-[(3,5-Dimethyl-1H-pyrazol-4-yl)thio]-9-fluoro-3,6-dihydro-7H-benzo[h]imidazo[4,5-
f]isoquinolin-7-one;
9-Fluoro-2-{[(5-hydroxy-1H-pyrazol-3-yl)methyl]thio}-3,6-dihydro-7H-
benzo[h]imidazo[4,5-f]isoquinolin-7-one;
9-Fluoro-2-[(2,3,5,6-tetrafluoropyridin-4-yl)thio]-3,6-dihydro-7H-benzo[h]imidazo[4,5-
f]isoquinolin-7-one;
2-[(2,6-Diamino-3,5-difluoropyridin-4-yl)thio]-9-fluoro-3,6-dihydro-7H-
benzo[h]imidazo[4,5-f]isoquinolin-7-one;
2-[(2-Amino-3,5,6-trifluoropyridin-4-yl)thio]-9-fluoro-3,6-dihydro-7H-
benzo[h]imidazo[4,5-f]isoquinolin-7-one;
2-(benzylthio)-9-fluoro-3,6-dihydro-7H-benzo[h]imidazo[4,5-f]isoquinolin-7-one;
2-(benzylsulfinyl)-9-fluoro-3,6-dihydro-7H-benzo[h]imidazo[4,5-f]isoquinolin-7-one;
2-(benzylsulfonyl)-9-fluoro-3,6-dihydro-7H-benzo[h]imidazo[4,5-f]isoquinolin-7-one;
3-{[(9-fluoro-7-oxo-6,7-dihydro-3H-benzo[h]imidazo[4,5-f]isoquinolin-2-
yl)thio]methyl}benzonitrile;
9-fluoro-2-[(2-methoxyethyl)thio]-3,6-dihydro-7H-benzo[h]imidazo[4,5-f]isoquinolin-7-
one;
9-fluoro-2-[(2-oxotetrahydrofiαran-3-yl)thio]-3,6-dihydro-7H-benzo[h]imidazo[4,5-
f]isoquinolin-7-one;
2-{2-[(9-fluoro-7-oxo-6,7-dihydro-3H-benzo[h]imidazo[4,5-f]isoquinolin-2-
yl)thio]ethyl}-1H-isoindole-1,3(2H)-dione;
9-fluoro-2-[(pyridin-3-ylmethyl)thio]-3,6-dihydro-7H-benzo[h]imidazo[4,5-
f]isoquinolin-7-one;
2-[(1-ethylpropyl)thio]-9-fluoro-3,6-dihydro-7H-benzo[h]imidazo[4,5-f]isoquinolin-7-
one;
2-[(1-ethylpropyl)sulfinyl]-9-fluoro-3,6-dihydro-7H-benzo[h]imidazo[4,5-f]isoquinolin-
7-one;
2-[(1-ethylpropyl)sulfonyl]-9-fluoro-3,6-dihydro-7H-benzo[h]imidazo[4,5-f]isoquinolin-
7-one;
2-[(3,5-dimethoxybenzyl)thio]-9-fluoro-3,6-dihydro-7H-benzo[h]imidazo[4,5-
f]isoquinolin-7-one;
ethyl[(9-fluoro-7-oxo-6,7-dihydro-3H-benzo[h]imidazo[4,5-f]isoquinolin-2-
yl)thio]acetate;
9-fluoro-2-(isopropylthio)-3,6-dihydro-7H-benzo[h]imidazo[4,5-f]isoquinolin-7-one;
[(9-fluoro-7-oxo-6,7-dihydro-3H-benzo[h]imidazo[4,5-f]isoquinolin-2-yl)thio]acetic
acid;
9-fluoro-2-[(1-phenylethyl)thio]-3,6-dihydro-7H-benzo[h]imidazo[4,5-f]isoquinolin-7-
one;
[(9-fluoro-7-oxo-6,7-dihydro-3H-benzo[h]imidazo[4,5-f]isoquinolin-2-
yl)thio]acetonitrile;
[(9-fluoro-7-oxo-6,7-dihydro-3H-benzo[h]imidazo[4,5-f]isoquinolin-2-yl)sulfinyl]acetonitrile;
2-[(9-fluoro-7-oxo-6,7-dihydro-3H-benzo[h]imidazo[4,5-f]isoquinolin-2-yl)thio]-N-
phenylpropanamide;
9-fluoro-2-[(3-hydroxypropyl)thio]-3,6-dihydro-7H-benzo[h]imidazo[4,5-f]isoquinolin-
7-one;
9-fluoro-2-[(3,3,3-trifluoro-2-oxopropyl)thio]-3,6-dihydro-7H-benzo[h]imidazo[4,5-
f]isoquinolin-7-one;
ethyl4-[(9-fluoro-7-oxo-6,7-dihydro-3H-benzo[h]imidazo[4,5-f]isoquinolin-2-
yl)thio]butanoate;
2-[(2-aminoethyl)thio]-9-fluoro-3,6-dihydro-7H-benzo[h]imidazo[4,5-f]isoquinolin-7-
one;
2-[(cyclohexylmethyl)thio]-9-fluoro-3,6-dihydro-7H-benzo[h]imidazo[4,5-f]isoquinolin-
7-one;
2-[(9-fluoro-7-oxo-6,7-dihydro-3H-benzo[h]imidazo[4,5-f]isoquinolin-2-
yl)thio]acetamide;
2-(cyclohexylthio)-9-fluoro-3,6-dihydro-7H-benzo[h]imidazo[4,5-f]isoquinolin-7-one;
2-[(2,2-dimethoxyethyl)thio]-9-fluoro-3,6-dihydro-7H-benzo[h]imidazo[4,5-
f]isoquinolin-7-one;
2-[(3,3-dimethyl-2-oxobutyl)thio]-9-fluoro-3,6-dihydro-7H-benzo[h]imidazo[4,5-
f]isoquinolin-7-one;
ethyl2-[(9-fluoro-7-oxo-6,7-dihydro-3H-benzo[h]imidazo[4,5-f]isoquinolin-2-
yl)thio]propanoate;
4-[(9-fluoro-7-oxo-6,7-dihydro-3H-benzo[h]imidazo[4,5-f]isoquinolin-2-
yl)thio]butanenitrile;
ethyl2-[(9-fluoro-7-oxo-6,7-dihydro-3H-benzo[h]imidazo[4,5-f]isoquinolin-2-
yl)thio]butanoate;
2-[(9-fluoro-7-oxo-6,7-dihydro-3H-benzo[h]imidazo[4,5-f]isoquinolin-2-yl)thio]-4-
hydroxy-N,N-dimethylbutanamide;
methyl3-{[(9-fluoro-7-oxo-6,7-dihydro-3H-benzo[h]imidazo[4,5-f]isoquinolin-2-
yl)thio]methyl}benzoate;
9-fluoro-2-[(tetrahydro-2H-pyran-2-ylmethyl)thio]-3,6-dihydro-7H-
benzo[h]imidazo[4,5-f]isoquinolin-7-one;
4-[(9-fluoro-7-oxo-6,7-dihydro-3H-benzo[h]imidazo[4,5-f]isoquinolin-2-yl)thio]butanoic
acid;
4-[(9-fluoro-7-oxo-6,7-dihydro-3H-benzo[h]imidazo[4,5-f]isoquinolin-2-
yl)thio]butanamide;
9-fluoro-7-oxo-6,7-dihydro-3H-benzo[h]imidazo[4,5-f]isoquinolin-2-yl thiocyanate;
2-[(9-fluoro-7-oxo-6,7-dihydro-3H-benzo[h]imidazo[4,5-f]isoquinolin-2-
yl)thio]propanenitrile;
9-fluoro-2-[(2-hydroxy-2-methylpropyl)thio]-3,6-dihydro-7H-benzo[h]imidazo[4,5-
f]isoquinolin-7-one;
9-fluoro-2-[(3-hydroxy-2,2-dimethylpropyl)thio]-3,6-dihydro-7H-benzo[h]imidazo[4,5-
f]isoquinolin-7-one;
9-fluoro-2-[(2-oxocyclopentyl)thio]-3,6-dihydro-7H-benzo[h]imidazo[4,5-f]isoquinolin-
7-one;
2-[(1,3-dioxolan-2-ylmethyl)thio]-9-fluoro-3,6-dihydro-7H-benzo[h]imidazo[4,5-
f]isoquinolin-7-one;
N-ethyl-2-[(9-fluoro-7-oxo-6,7-dihydro-3H-benzo[h]imidazo[4,5-f]isoquinolin-2-
yl)thio]-4-hydroxybutanamide;
9-fluoro-2-[(2-hydroxyethyl)thio]-3,6-dihydro-7H-benzo[h]imidazo[4,5-f]isoquinolin-7-
one;
9-fluoro-2-(piperidin-4-ylthio)-3,6-dihydro-7H-benzo[h]imidazo[4,5-f]isoquinolin-7-one;
3-[(9-fluoro-7-oxo-6,7-dihydro-3H-benzo[h]imidazo[4,5-f]isoquinolin-2-
yl)thio]propanenitrile;
9-fluoro-2-[(3-oxocyclohex-1-en-1-yl)thio]-3,6-dihydro-7H-phenanthro[9,10-
d]imidazol-7-one;
9-fluoro-2-(pyridin-4-ylthio)-3,6-dihydro-7H-benzo[h]imidazo[4,5-f]isoquinolin-7-one;
9-fluoro-2-(1H-pyrazol-4-ylthio)-3,6-dihydro-7H-benzo[h]imidazo[4,5-f]isoquinolin-7-
one;
9-fluoro-2-[(2-hydroxy-2-methylpropyl)sulfinyl]-3,6-dihydro-7H-benzo[h]imidazo[4,5-
f]isoquinolin-7-one;
3-[(9-fluoro-7-oxo-6,7-dihydro-3H-benzo[h]imidazo[4,5-f]isoquinolin-2-
yl)sulfinyl]propanenitrile;
9-fluoro-2-[(2-hydroxy-3,3-dimethylbutyl)thio]-3,6-dihydro-7H-benzo[h]imidazo[4,5-
f]isoquinolin-7-one;
9-fluoro-2-[(2-oxopropyl)thio]-3,6-dihydro-7H-benzo[h]imidazo[4,5-f]isoquinolin-7-
one;
9-fluoro-2-(pyridin-4-ylsulfinyl)-3,6-dihydro-7H-benzo[h]imidazo[4,5-f]isoquinolin-7-
one;
4-[(9-fluoro-7-oxo-6,7-dihydro-3H-benzo[h]imidazo[4,5-f]isoquinolin-2-
yl)sulfinyl]butanenitrile;
9-fluoro-2-(pyrimidin-5-ylthio)-3,6-dihydro-7H-benzo[h]imidazo[4,5-f]isoquinolin-7-
one;
ethyl4-[(9-fluoro-7-oxo-6,7-dihydro-3H-benzo[h]imidazo[4,5-f]isoquinolin-2-yl)thio]-3-
oxobutanoate;
3-[(9-fluoro-7-oxo-6,7-dihydro-3H-benzo[h]imidazo[4,5-f]isoquinolin-2-yl)thio]-2-
methylpropanenitrile;
3-[(9-fluoro-7-oxo-6,7-dihydro-3H-benzo[h]imidazo[4,5-f]isoquinolin-2-yl)sulfinyl]-2-
methylpropanenitrile;
9-fluoro-2-[(2-hydroxypropyl)thio]-3,3a,6,11b-tetrahydro-7H-benzo[h]imidazo[4,5-
f]isoquinolin-7-one;
9-fluoro-2-(isoxazol-4-ylthio)-3,6-dihydro-7H-benzo[h]imidazo[4,5-f]isoquinolin-7-one;
3-[(9-fluoro-7-oxo-6,7-dihydro-3H-benzo[h]imidazo[4,5-f]isoquinolin-2-
yl)thio]propanamide;
2-[(3,5-dichloropyridin-4-yl)thio]-9-fluoro-3,6-dihydro-7H-benzo[h]imidazo[4,5-
f]isoquinolin-7-one;
2-[(4,6-dimethoxy-1,3,5-triazin-2-yl)thio]-9-fluoro-3,6-dihydro-7H-benzo[h]imidazo[4,5-
f]isoquinolin-7-one;
9-fluoro-2-(pyrimidin-2-ylthio)-3,6-dihydro-7H-benzo[h]imidazo[4,5-f]isoquinolin-7-
one;
2-[(2,2-dimethylpropyl)thio]-9-fluoro-3,6-dihydro-7H-benzo[h]imidazo[4,5-
f]isoquinolin-7-one;
9-fluoro-2-(isobutylthio)-3,6-dihydro-7H-benzo[h]imidazo[4,5-f]isoquinolin-7-one;
2-[(2,2-dimethylpropyl)sulfinyl]-9-fluoro-3,6-dihydro-7H-benzo[h]imidazo[4,5-
f]isoquinolin-7-one;
2-(cyclohexylsulfinyl)-9-fluoro-3,6-dihydro-7H-benzo[h]imidazo[4,5-f]isoquinolin-7-
one;
9-fluoro-2-(pyrazin-2-ylthio)-3,6-dihydro-7H-benzo[h]imidazo[4,5-f]isoquinolin-7-one;
9-fluoro-2-[(3-fluoropyridin-4-yl)thio]-3,6-dihydro-7H-benzo[h]imidazo[4,5-
f]isoquinolin-7-one;
2-[(6-chloropyrimidin-4-yl)thio]-9-fluoro-3,6-dihydro-7H-benzo[h]imidazo[4,5-
f]isoquinolin-7-one;
2-[(2,6-dimethoxypyrimidin-4-yl)thio]-9-fluoro-3,6-dihydro-7H-benzo[h]imidazo[4,5-
f]isoquinolin-7-one;
2-[(2,6-dichloropyridin-4-yl)thio]-9-fluoro-3,6-dihydro-7H-benzo[h]imidazo[4,5-
f]isoquinolin-7-one;
2-[(6-chloropyridazin-3-yl)thio]-9-fluoro-3,6-dihydro-7H-benzo[h]imidazo[4,5-
f]isoquinolin-7-one;
2-[(3-amino-6-bromopyrazin-2-yl)thio]-9-fluoro-3,6-dihydro-7H-benzo[h]imidazo[4,5-
f]isoquinolin-7-one;
9-fluoro-2-(quinolin-4-ylthio)-3,6-dihydro-7H-benzo[h]imidazo[4,5-f]isoquinolin-7-one;
2-[(6-chloropyrazin-2-yl)thio]-9-fluoro-3,6-dihydro-7H-benzo[h]imidazo[4,5-
f]isoquinolin-7-one;
2-[(2,6-dichloropyrimidin-4-yl)thio]-9-fluoro-3,6-dihydro-7H-benzo[h]imidazo[4,5-
f]isoquinolin-7-one;
9-fluoro-2-[(1-oxidopyridin-4-yl)thio]-3,6-dihydro-7H-benzo[h]imidazo[4,5-
f]isoquinolin-7-one;
2-[(2,6-diaminopyrimidin-4-yl)thio]-9-fluoro-3,6-dihydro-7H-benzo[h]imidazo[4,5-
f]isoquinolin-7-one;
9-fluoro-2-[(1-methyl-1H-pyrazol-4-yl)thio]-3,6-dihydro-7H-benzo[h]imidazo[4,5-
f]isoquinolin-7-one;
3-[(9-fluoro-7-oxo-6,7-dihydro-3H-benzo[h]imidazo[4,5-f]isoquinolin-2-
yl)thio]pyrazine-2-carbonitrile;
9-fluoro-2-[(2-methylpyrimidin-5-yl)thio]-3,6-dihydro-7H-benzo[h]imidazo[4,5-
f]isoquinolin-7-one;
2-[(5-chloro-3-hydroxypyridazin-4-yl)thio]-9-fluoro-3,6-dihydro-7H-
benzo[h]imidazo[4,5-f]isoquinolin-7-one;
2-[(3,5-dichloropyridazin-4-yl)thio]-9-fluoro-3,6-dihydro-7H-benzo[h]imidazo[4,5-
f]isoquinolin-7-one;
9-fluoro-2-[(1-oxidopyridin-2-yl)thio]-3,6-dihydro-7H-benzo[h]imidazo[4,5-
f]isoquinolin-7-one;
9-fluoro-2-[(1H-tetrazol-5-ylmethyl)thio]-3,6-dihydro-7H-benzo[h]imidazo[4,5-
f]isoquinolin-7-one trifluoroacetate;
9-fluoro-2-[(6-hydroxypyridin-3-yl)thio]-3,6-dihydro-7H-benzo[h]imidazo[4,5-
f]isoquinolin-7-one;
2-[(2-amino-6-chloropyridin-4-yl)thio]-9-fluoro-3,6-dihydro-7H-benzo[h]imidazo[4,5-
f]isoquinolin-7-one trifluoroacetate;
2-[(6-aminopyrimidin-4-yl)thio]-9-fluoro-3,6-dihydro-7H-benzo[h]imidazo[4,5-
f]isoquinolin-7-one;
2-[(6-aminopyrimidin-4-yl)thio]-9-fluoro-3,6-dihydro-7H-benzo[h]imidazo[4,5-
f]isoquinolin-7-one;
9-fluoro-2-(pyridin-3-ylthio)-3,6-dihydro-7H-benzo[h]imidazo[4,5-f]isoquinolin-7-one
trifluoroacetate;
9-fluoro-2-(phenylthio)-3,6-dihydro-7H-benzo[h]imidazo[4,5-f]isoquinolin-7-one
trifluoroacetate;
2-[(2-chloropyrimidin-4-yl)thio]-9-fluoro-3,6-dihydro-7H-benzo[h]imidazo[4,5-
f]isoquinolin-7-one;
9-fluoro-2-(1H-tetrazol-5-ylthio)-3,6-dihydro-7H-benzo[h]imidazo[4,5-f]isoquinolin-7-
one;
2-[(5-bromopyridin-3-yl)thio]-9-fluoro-3,6-dihydro-7H-benzo[h]imidazo[4,5-
f]isoquinolin-7-one trifluoroacetate;
2-[(3-aminophenyl)thio]-9-fluoro-3,6-dihydro-7H-benzo[h]imidazo[4,5-f]isoquinolin-7-
one trifluoroacetate;
4-[(9-fluoro-7-oxo-6,7-dihydro-3H-benzo[h]imidazo[4,5-f]isoquinolin-2-
yl)thio]pyridine-2-carbonitrile;
9-fluoro-2-[(5-methoxypyridin-3-yl)thio]-3,6-dihydro-7H-benzo[h]imidazo[4,5-
f]isoquinolin-7-one trifluoroacetate;
2-[(2-aminopyrimidin-4-yl)thio]-9-fluoro-3,6-diliydro-7H-benzo[li]imidazo[4,5-
f]isoquinolin-7-one;
5-[(9-fluoro-7-oxo-6,7-diliydro-3H-benzo[li]imidazo[4,5-f]isoquinolin-2-yl)thio]-N-(2-
hydroxyethyl)nicotinamide trifluoroacetate(salt);
methyl4-chloro-5-[(9-fluoro-7-oxo-6,7-dihydro-3H-benzo[h]imidazo[4,5-f]isoqumolin-
2-yl)thio]pyridine-2-carboxylate;
9-fluoro-2-[(6-fluoropyridin-3-yl)thio]-3,6-diliydro-7H-benzo[li]imidazo[4,5-
f]isoquinolin-7-one trifluoroacetate;
2-[(6-aminopyridin-3-yl)thio]-9-fluoro-3,6-diliydro-7H-benzo[li]imidazo[4,5-
f]isoquinolin-7-one trifluoroacetate;
9-fluoro-2-[(6-methoxypyridin-3-yl)thio]-3,6-diliydro-7H-benzo[li]imidazo[4,5-
f]isoqumolin-7-one trifluoroacetate;
9-fluoro-2-[(2-fluoropyridin-4-yl)thio]-3,6-diliydro-7H-benzo[li]imidazo[4,5-
f]isoquinolin-7-one;
9-fluoro-2-(quinolin-3-ylthio)-3,6-dihydro-7H-benzo[li]imidazo[4,5-f]isoqumolin-7-one
trifluoroacetate;
5-[(9-fluoro-7-oxo-6,7-diliydro-3H-benzo[li]imidazo[4,5-f]isoquinolin-2-
yl)thio]nicotinamide trifluoroacetate;
2-[(2-aminopyridin-4-yl)thio]-9-fluoro-3,6-diliydro-7H-benzo[li]imidazo[4,5-
f]isoquinolin-7-one;
9-fluoro-2-[(2-methoxypyridin-4-yl)thio]-3,6-diliydro-7H-benzo[li]imidazo[4,5-
f]isoqumolin-7-one;
9-fluoro-2-{[5-(morpholin-4-ylcarbonyl)pyridin-3-yl]thio}-3,6-diliydro-7H-
benzo[h]imidazo[4,5-f]isoquinolin-7-one trifluoroacetate;
5-[(9-fluoro-7-oxo-6,7-diliydro-3H-benzo[li]imidazo[4,5-f]isoquinolin-2-yl)thio]-N-[2-
(tetrahydro-2H-pyran-4-yl)ethyl]nicotinamide;
9-fluoro-2-({5-[(4-methylpiperazin-1-yl)carbonyl]pyridin-3-yl}thio)-3,6-dihydro-7H-
benzo[h]imidazo[4,5-f]isoquinolin-7-one;
9-fluoro-2-({2-[(2-morpholin-4-ylethyl)amino]pyridin-4-yl}thio)-3,6-dihydro-7H-
benzo[h]imidazo[4,5-f]isoquinolin-7-one;
2-{[2-(dimethylamino)pyridin-4-yl]thio}-9-fluoro-3,6-dihydro-7H-benzo[h]imidazo[4,5-
f]isoquinolin-7-one;
9-fluoro-2-({2-[(2-hydroxyethyl)amino]pyridin-4-yl}thio)-3,6-dihydro-7H-
benzo[h]imidazo[4,5-f]isoquinolin-7-one; methyl4-[(9-fluoro-7-oxo-6,7-dihydro-3H-
benzo[h]imidazo[4,5-f]isoquinolin-2-yl)thio]pyridine-2-carboxylate;
9-fluoro-2-(isoquinolin-4-ylthio)-3,6-dihydro-7H-benzo[h]imidazo[4,5-f]isoquinolin-7-
one trifluoroacetate;
9-fluoro-2-({6-[(2-hydroxyethyl)amino]pyridin-3-yl}thio)-3,6-dihydro-7H-
benzo[h]imidazo[4,5-f]isoquinolin-7-one trifluoroacetate;
9-fluoro-2-({6-[(2-morpholin-4-ylethyl)amino]pyridin-3-yl}thio)-3,6-dihydro-7H-
benzo[h]imidazo[4,5-f]isoquinolin-7-one trifluoroacetate;
4-[(9-fluoro-7-oxo-6,7-dihydro-3H-benzo[h]imidazo[4,5-f]isoquinolin-2-
yl)thio]benzamide;
2-[(5-aminopyridin-3-yl)thio]-9-fluoro-3,6-dihydro-7H-benzo[h]imidazo[4,5-
f]isoquinolin-7-one;
9-fluoro-2-{[4-(1H-imidazol-1-yl)phenyl]thio}-3,6-dihydro-7H-benzo[h]imidazo[4,5-
f]isoquinolin-7-one trifluoroacetate;
9-fluoro-2-[(3-hydroxyphenyl)thio]-3,6-dihydro-7H-benzo[h]imidazo[4,5-f]isoquinolin-
7-one trifluoroacetate (salt);
4-[(9-fluoro-7-oxo-6,7-dihydro-3H-benzo[h]imidazo[4,5-f]isoquinolin-2-yl)thio]-N-(2-
hydroxyethyl)benzamide trifluoroacetate (salt);
3-[(9-fluoro-7-oxo-6,7-dihydro-3H-benzo[h]imidazo[4,5-f]isoquinolin-2-yl)thio]-N-(2-
hydroxyethyl)benzamide trifluoroacetate (salt);
3-[(9-fluoro-7-oxo-6,7-dihydro-3H-benzo[h]imidazo[4,5-f]isoquinolin-2-
yl)thio]benzonitrile trifluoroacetate;
9-fluoro-2-[(5-morpholin-4-ylpyridin-3-yl)thio]-3,6-dihydro-7H-benzo[h]imidazo[4,5-
f]isoquinolin-7-one trifluoroacetate;
{3-[(9-fluoro-7-oxo-6,7-dihydro-3H-benzo[h]imidazo[4,5-f]isoquinolin-2-
yl)thio]phenoxy}acetonitrile trifluoroacetate;
N-{3-[(9-fluoro-7-oxo-6,7-dihydro-3H-benzo[h]imidazo[4,5-f]isoqumolin-2-
yl)thio]phenyl}methanesulfonamide trifluoroacetate;
2-cyano-N-{3-[(9-fluoro-7-oxo-6,7-diliydro-3H-benzo[li]imidazo[4,5-f]isoquinolin-2-
yl)thio]phenyl}acetamide trifluoroacetate;
N′-{3-[(9-fluoro-7-oxo-6,7-dihydro-3H-benzo[h]imidazo[4,5-f]isoquinolin-2-
yl)thio]phenyl}-N,N-dimethylsulfamide;
N-{3-[(9-fluoro-7-oxo-6,7-dihydro-3H-benzo[h]imidazo[4,5-f]isoquinolin-2-
yl)thio]phenyl}ethanesulfonamide;
2-(dimethylamino)-N-{3-[(9-fluoro-7-oxo-6,7-dihydro-3H-benzo[h]imidazo[4,5-
f]isoquinolin-2-yl)thio]phenyl}acetamide trifluoroacetate;
N-ethyl-N′-{3-[(9-fluoro-7-oxo-6,7-dihydro-3H-benzo[h]imidazo[4,5-f]isoquinolin-2-
yl)thio]phenyl}urea trifluoroacetate;
2-tert-butyl-10-fluorobenzo[c]imidazo[1,2-a]-1,6-naphthyridin-8(7H)-one;
10-Fluoro-2-(4-hydroxycyclohexyl)benzo[c]imidazo[1,2-a]-1,6-naphthyridin-8(7H)-
one; trans-4-(10-Fluoro-8-oxo-7,8-dihydrobenzo[c]imidazo[1,2-a]-1,6-naphthyridin-2-
yl)cyclohexyl(dimethylamino)acetate;
10-fluoro-2-(1-methylcyclopropyl)benzo[c]imidazo[1,2-a]-1,6-naphthyridin-8(7H)-
one;
2-cyclopropyl-10-fluorobenzo[c]imidazo[1,2-a]-1,6-naphthyridin-8(7H)-one;
2-cyclobutyl-10-fluorobenzo[c]imidazo[1,2-a]-1,6-naphthyridin-8(7H)-one;
2-ethyl-10-fluorobenzo[c]imidazo[1,2-a]-1,6-naphthyridin-8(7H)-one; ethyl2-ethyl-2-(10-
fluoro-8-oxo-7,8-dihydrobenzo[c]imidazo[1,2-a]-1,6-naphthyridin-2-yl)butanoate;
2-[1-ethyl-1-(hydroxymethyl)propyl]-10-fluorobenzo[c]imidazo[1,2-a]-1,6-
naphthyridin-8(7H)-one;
2-(1-ethylpropyl)-10-fluorobenzo[c]imidazo[1,2-a]-1,6-naphthyridin-8(7H)-one;
10-fluoro-2-(4-oxocyclohexyl)benzo[c]imidazo[1,2-a]-1,6-naphthyridin-8(7H)-one;
10-fluoro-2-methylbenzo[c]imidazo[1,2-a]-1,6-naphthyridin-8(7H)-one;
10-fluoro-2-(cis-4-methoxycyclohexyl)benzo[c]imidazo[1,2-a]-1,6-naphthyridin-8(7H)-
one;
10-fluoro-2-(trans-4-methoxycyclohexyl)benzo[c]imidazo[1,2-a]-1,6-naphthyridin-
8(7H)-one;
10-fluoro-2-[4-(hydroxylmino)cyclohexyl]benzo[c]imidazo[1,2-a]-1,6-naphthyridin-
8(7H)-one;
2-(1-acetylpiperidin-4-yl)-10-fluorobenzo[c]imidazo[1,2-a]-1,6-naphthyridin-8(7H)-
one;
2-amino-9-fluorobenzo[h][1,3]thiazolo[5,4-f]isoquinolin-7(6H)-one;
9-fluoro-2-(4-hydroxypiperidin-1-yl)benzo[h][1,3]thiazolo[5,4-f]isoquinolin-7(6H)-one;
9-fluoro-2-{[(1S)-1-(hydroxymethyl)-3-methylbutyl]amino}benzo[h][1,3]thiazolo[5,4-
f]isoquinolin-7(6H)-one;
9-fluoro-2-{[(1R)-2-hydroxy-1-phenylethyl]amino}benzo[h][1,3]thiazolo[5,4-
f]isoquinolin-7(6H)-one;
(2S)-2-[(9-fluoro-7-oxo-6,7-dihydrobenzo[h][1,3]thiazolo[5,4-f]isoquinolin-2-yl)amino]-
4-methoxy-N-methylbutanamide;
9-fluoro-2-(4-oxopiperidin-1-yl)benzo[h][1,3]thiazolo[5,4-f]isoquinolin-7(6H)-one;
9-fluoro-2-{[(1R)-1-(hydroxymethyl)-3-methylbu1yl]amino}benzo[h][1,3]thiazolo[5,4-
f]isoquinolin-7(6H)-one;
9-fluoro-2-(3-hydroxypiperidin-1-yl)benzo[h][1,3]thiazolo[5,4-f]isoquinolin-7(6H)-one;
9-fluoro-2-{[(1S)-2-hydroxy-1-phenylethyl]amino}benzo[h][1,3]thiazolo[5,4-
f]isoquinolin-7(6H)-one;
9-fluoro-2-(3-hydroxypyrrolidin-1-yl)benzo[h][1,3]thiazolo[5,4-f]isoquinolin-7(6H)-one;
9-fluorobenzo[h][1,3]thiazolo[5,4-f]isoquinolin-7(6H)-one;
9-fluoro-2-(1-hydroxy-4-oxocyclohexyl)benzo[h][1,3]thiazolo[5,4-f]isoquinolin-7(6H)-
one;
2-[4-(ethoxyimino)piperidin-1-yl]-9-fluorobenzo[h][1,3]thiazolo[5,4-f]isoquinolin-7(6H)-
one;
9-fluoro-2-[4-(hydroxyimino)piperidin-1-yl]benzo[h][1,3thiazolo[5,4-f]isoquinolin-
7(6H)-one;
4-(9-fluoro-7-oxo-6,7-dihydrobenzo[h][1,3]thiazolo[5,4-f]isoquinolin-2-yl)butanenitrile;
2-(1,4-dihydroxycyclohexyl)-9-fluorobenzo[h][1,3]thiazolo[5,4-f]isoquinolin-7(6H)-one;
2-(1-acetyl-4-hydroxypiperidin-4-yl)-9-fluorobenzo[h][1,3]thiazolo[5,4-f]isoquinolin-
7(6H)-one;
9-fluoro-2-(4-hydroxy-1-isobutyrylpiperidin-4-yl)benzo[h][1,3]thiazolo[5,4-
f]isoquinolin-7(6H)-one;
9-fluoro-2-isonicotinoylbenzo[h][1,3]thiazolo[5,4-f]isoquinolin-7(6H)-one;
9-fluoro-2-(1-oxidoisonicotinoyl)benzo[h][1,3]thiazolo[5,4-f]isoquinolin-7(6H)-one;
9-fluoro-2-phenylbenzo[h][1,3]thiazolo[5,4-f]isoquinolin-7(6H)-one;
2-ethyl-9-fluorobenzo[h][1,3]thiazolo[5,4-f]isoquinolin-7(6H)-one;
9-fluoro-2-morpholin-4-ylbenzo[h][1,3]thiazolo[5,4-f]isoquinolin-7(6H)-one;
9-fluoro-2-[(pyridin-3-ylmethyl)amino]benzo[h][1,3]thiazolo[5,4-f]isoquinolin-7(6H)-
one;
9-fluoro-2-[(3-morpholin-4-ylpropyl)amino]benzo[h][1,3]thiazolo[5,4-f]isoquinolin-
7(6H)-one;
9-fluoro-2-[(3-methoxybenzyl)amino]benzo[h][1,3]thiazolo[5,4-f]isoquinolin-7(6H)-one;
9-fluoro-2-{[(1R)-1-(3-methoxyphenyl)ethyl]amino}benzo[h][1,3]thiazolo[5,4-
f]isoquinolin-7(6H)-one;
9-fluoro-2-{[(1S)-1-(3-methoxyphenyl)ethyl]amino}benzo[h][1,3]thiazolo[5,4-
f]isoquinolin-7(6H)-one;
9-fluoro-2-[(3-hydroxypropyl)amino]benzo[h][1,3]thiazolo[5,4-f]isoquinolin-7(6H)-one;
9-fluoro-2-[(2-hydroxyethyl)(methyl)amino]benzo[h][1,3]thiazolo[5,4-f]isoquinolin-
7(6H)-one;
9-fluoro-2-{[2-(4-hydroxyphenyl)ethyl]amino}benzo[h][1,3]thiazolo[5,4-f]isoquinolin-
7(6H)-one;
9-fluoro-2-[(2-methoxy-1-methylethyl)amino]benzo[h][1,3]thiazolo[5,4-f]isoquinolin-
7(6H)-one;
N,N-diethyl-1-(9-fluoro-7-oxo-6,7-dihydrobenzo[h][1,3]thiazolo[5,4-f]isoquinolin-2-
yl)piperidine-3-carboxamide;
9-fluoro-2-[(3-phenylpropyl)amino]benzo[h][1,3]thiazolo[5,4-f]isoquinolin-7(6H)-one;
9-fluoro-2-{[(1R)-1-(4-methoxyphenyl)ethyl]amino}benzo[h][1,3]thiazolo[5,4-
f]isoquinolin-7(6H)-one;
9-fluoro-2-{[(1S)-1-(4-methoxyphenyl)ethyl]amino}benzo[h][1,3]thiazolo[5,4-
f]isoquinolin-7(6H)-one;
9-fluoro-2-(pyridin-3-ylamino)benzo[h][1,3]thiazolo[5,4-f]isoquinolin-7(6H)-one;
9-fluoro-2-(4-pyridin-2-ylpiperazin-1-yl)benzo[h][1,3]thiazolo[5,4-f]isoquinolin-7(6H)-
one;
9-fluoro-2-{[3-(1H-imidazol-1-yl)propyl]amino}benzo[h][1,3]thiazolo[5,4-f]isoquinolin-
7(6H)-one;
9-fluoro-2-[(2-morpholin-4-ylethyl)amino]benzo[h][1,3]thiazolo[5,4-f]isoquinolin-7(6H)-
one;
9-fluoro-2-[(2-methoxyethyl)amino]benzo[h][1,3]thiazolo[5,4-f]isoquinolin-7(6H)-one;
9-fluoro-2-[(tetrahydrofuran-2-ylmethyl)amino]benzo[h][1,3]thiazolo[5,4-f]isoquinolin-
7(6H)-one;
9-fluoro-2-[(2-pyridin-2-ylethyl)amino]benzo[h][1,3]thiazolo[5,4-f]isoquinolin-7(6H)-
one;
9-fluoro-2-{[2-(1H-imidazol-4-yl)ethyl]amino}benzo[h][1,3]thiazolo[5,4-f]isoquinolin-
7(6H)-one;
9-fluoro-2-[4-(2-hydroxyethyl)piperazin-1-yl]benzo[h][1,3]thiazolo[5,4-f]isoquinolin-
7(6H)-one;
2-{[3-(dimethylamino)propyl]amino}-9-fluorobenzo[h][1,3]thiazolo[5,4-f]isoquinolin-
7(6H)-one;
9-fluoro-2-(4-hydroxy-4-pyridin-2-ylpiperidin-1-yl)benzo[h][1,3]thiazolo[5,4-
f]isoquinolin-7(6H)-one;
2-[bis(2-methoxyethyl)amino]-9-fluorobenzo[h][1,3]thiazolo[5,4-f]isoquinolin-7(6H)-
one;
(9-fluoro-7-oxo-6,7-dihydrobenzo[h][1,3]thiazolo[5,4-f]isoquinolin-2-yl)acetonitrile;
2-{benzyl[2-(dimethylamino)ethyl]amino}-9-fluorobenzo[h][1,3]thiazolo[5,4-
f]isoquinolin-7(6H)-one;
9-fluoro-2-{[3-(trifluoromethyl)phenyl]amino}benzo[h][1,3]thiazolo[5,4-f]isoquinolin-
7(6H)-one;
9-fluoro-2-[(2-methoxyphenyl)amino]benzo[h][1,3]thiazolo[5,4-f]isoquinolin-7(6H)-one;
9-fluoro-2-(propylamino)benzo[h][1,3]thiazolo[5,4-f]isoquinolin-7(6H)-one;
9-fluoro-2-(isopropylamino)benzo[h][1,3]thiazolo[5,4-f]isoquinolin-7(6H)-one;
3-[(9-fluoro-7-oxo-6,7-dihydrobenzo[h][1,3]thiazolo[5,4-f]isoquinolin-2-
yl)amino]benzonitrile;
9-fluoro-2-[(3-hydroxyphenyl)amino]benzo[h][1,3]thiazolo[5,4-f]isoquinolin-7(6H)-one;
9-fluoro-2-[(trans-4-hydroxy-4-pyridin-2-ylcyclohexyl)amino]benzo[h][1,3]thiazolo[5,4-
f]isoquinolin-7(6H)-one;
9-fluoro-2-[cis-4-hydroxy-3-(hydroxymethyl)piperidin-1-yl]benzo[h][1,3]thiazolo[5,4-
f]isoquinolin-7(6H)-one;
9-fluoro-2-[trans-4-hydroxy-3-(hydroxymethyl)piperidin-1-yl]benzo[h][1,3]thiazolo[5,4-
f]isoquinolin-7(6H)-one;
9-fluoro-2-[(2-hydroxyethyl)amino]benzo[h][1,3]thiazolo[5,4-f]isoquinolin-7(6H)-one;
9-fluoro-2-[(4-hydroxybenzyl)amino]benzo[h][1,3]thiazolo[5,4-f]isoquinolin-7(6H)-one;
9-fluoro-2-[4-(hydroxymethyl)piperidin-1-yl]benzo[h][1,3]thiazolo[5,4-f]isoquinolin-
7(6H)-one;
9-fluoro-2-[3-(hydroxymethyl)piperidin-1-yl]benzo[h][1,3]thiazolo[5,4-f]isoquinolin-
7(6H)-one;
9-fluoro-2-[(2-hydroxy-1-methylethypamino]benzo[h][1,3]thiazolo[5,4-f]isoquinolin-
7(6H)-one;
9-fluoro-2-[(2S)-2-(hydroxymethyl)pyrrolidin-1-yl]benzo[h][1,3]thiazolo[5,4-
f]isoquinolin-7(6H)-one;
9-fluoro-2-[(2R)-2-(hydroxymethyl)pyrrolidin-1-yl]benzo[h][1,3]thiazolo[5,4-
b]isoquinolin-7(6H)-one;
2-(4-acetylpiperazin-1-yl)-9-fluorobenzo[h][1,3]thiazolo[5,4-f]isoquinolin-7(6H)-one;
9-fluoro-2-piperazin-1-ylbenzo[h][1,3]thiazolo[5,4-f]isoquinolin-7(6H)-one;
2-{4-[(dimethylamino)acetyl]piperazin-1-yl}-9-fluorobenzo[h][1,3]thiazolo[5,4-
f]isoquinolin-7(6H)-one;
9-fluoro-2-[4-(2-hydroxyethyl)piperidin-1-yl]benzo[h][1,3]thiazolo[5,4-f]isoquinolin-
7(6H)-one;
9-fluoro-2-{4(2R)-2-hydroxypropyl]piperazin-1-yl}benzo[h][1,3]thiazolo[5,4-
f]isoquinolin-7(6H)-one;
2-[(2,3-dihydroxypropyl)amino]-9-fluorobenzo[h][1,3]thiazolo[5,4-f]isoquinolin-7(6H)-
one;
4-(9-fluoro-7-oxo-6,7-dihydrobenzo[h][1,3]thiazolo[5,4-f]isoquinolin-2-yl)piperazine-1-
carboxamide;
methyl 4-(9-fluoro-7-oxo-6,7-dihydrobenzo[h][1,3]thiazolo[5,4-f]isoquinolin-2-
yl)piperazine-1-carboxylate;
ethyl 4-(9-fluoro-7-oxo-6,7-dihydrobenzo[h][1,3]thiazolo[5,4-f]isoquinolin-2-
yl)piperazine-1-carboxylate;
4-(9-fluoro-7-oxo-6,7-dihydrobenzo[h][1,3]thiazolo[5,4-f]isoquinolin-2-yl)piperazine-1-
carbaldehyde;
9-fluoro-2-[4-(1H-tetrazol-5-ylacetyl)piperazin-1-yl]benzo[h][1,3]thiazolo[5,4-
f]isoquinolin-7(6H)-one;
9-fluoro-2-(pyridin-2-ylamino)benzo[h][1,3]thiazolo[5,4-f]isoquinolin-7(6H)-one;
9-fluoro-2-(pyridin-4-ylamino)benzo[h][1,3]thiazolo[5,4-f]isoquinolin-7(6H)-one;
9-fluoro-2-[(3R,4R)-3-methoxy-4-(methylamino)pyrrolidin-1-
yl]benzo[h][1,3]thiazolo[5,4-f]isoquinolin-7(6H)-one;
9-fluoro-2-[(3S,4R)-3-methoxy-4-(methylamino)pyrrolidin-1-
yl]benzo[h][1,3]thiazolo[5,4-f]isoquinolin-7(6H)-one;
benzyl(3R,4R)-3-[(9-fluoro-7-oxo-6,7-dihydrobenzo[h][1,3]thiazolo[5,4-f]isoquinolin-2-
yl)(methyl)amino]-4-methoxypyrrolidine-1-carboxylate;
2-(1-ethyl-1-hydroxypropyl)-9-fluorobenzo[h][1,3]thiazolo[5,4-f]isoquinolin-7(6H)-one;
9-fluoro-2-(hydroxymethyl)benzo[h][1,3]thiazolo[5,4-f]isoquinolin-7(6H)-one;
2-(ethoxymethyl)-9-fluorobenzo[h][1,3]thiazolo[5,4-f]isoquinolin-7(6H)-one;
3-(9-fluoro-7-oxo-6,7-dihydrobenzo[h][1,3]thiazolo[5,4-f]isoquinolin-2-
yl)propanenitrile;
[1-(9-fluoro-7-oxo-6,7-dihydrobenzo[h][1,3]thiazolo[5,4-f]isoquinolin-2-yl)piperidin-4-
yl]acetonitrile;
1-(9-fluoro-7-oxo-6,7-dihydrobenzo[h][1,3]thiazolo[5,4-f]isoquinolin-2-yl)piperidine-4-
carbonitrile;
3-[(9-fluoro-7-oxo-6,7-dihydrobenzo[h][1,3]thiazolo[5,4-f]isoquinolin-2-
yl)methoxy]propanenitrile;
9-fluoro-2-{4-[2-(1H-1,2,4-triazol-1-yl)ethyl]piperidin-1-yl}benzo[h][1,3]thiazolo[5,4-
f]isoquinolin-7(6H)-one;
9-fluoro-2-[4-(1H-1,2,4-triazol-1-ylmethyl)piperidin-1-yl]benzo[h][1,3]thiazolo[5,4-
f]isoquinolin-7(6H)-one;
9-fluoro-2-[4-(1H-imidazol-1-ylmethyl)piperidin-1-yl]benzo[h][1,3]thiazolo[5,4-
f]isoquinolin-7(6H)-one;
3-[1-(9-fluoro-7-oxo-6,7-dihydrobenzo[h][1,3]thiazolo[5,4-f]isoquinolin-2-yl)piperidin-
4-yl]propanenitrile;
9-fluoro-2-{4-[2-(1H-imidazol-1-yl)ethyl]piperidin-1-yl}benzo[h][1,3]thiazolo[5,4-
f]isoquinolin-7(6H)-one;
2-{4-[(diethylamino)methyl]piperidin-1-yl}-9-fluorobenzo[h][1,3]-thiazolo[5,4-
f]isoquinolin-7(6H)-one;
9-fluoro-2-(pyridin-3-ylmethyl)benzo[h][1,3]thiazolo[5,4-f]isoquinolin-7(6H)-one;
9-fluoro-2-(pyridin-2-ylmethyl)benzo[h][1,3]thiazolo[5,4-f]isoquinolin-7(6H)-one;
9-fluoro-2-[(1-oxidopyridin-3-yl)methyl]benzo[h][1,3]thiazolo[5,4-f]isoquinolin-7(6H)-
one;
9-fluoro-2-(pyridin-4-ylmethyl)benzo[h][1,3]thiazolo[5,4-f]isoquinolin-7(6H)-one;
9-fluoro-2-[hydroxy(6-methoxypyridin-3-yl)methyl]benzo[h][1,3]thiazolo[5,4-
f]isoquinolin-7(6H)-one;
9-fluoro-2-[3-hydroxy-1-(pyridin-4-ylmethyl)piperidin-3-yl]benzo[h][1,3]thiazolo[5,4-
f]isoquinolin-7(6H)-one;
2-(1-acetyl-3-hydroxypiperidin-3-yl)-9-fluorobenzo[h][1,3]thiazolo[5,4-f]isoquinolin-
7(6H)-one;
9-fluoro-2-[(4-hydroxypiperidin-1-yl)methyl]benzo[h][1,3]thiazolo[5,4-f]isoquinolin-
7(6H)-one;
2-[(4-acetylpiperazin-1-yl)carbonyl]-9-fluorobenzo[h][1,3]thiazolo[5,4-f]isoquinolin-
7(6H)-one;
9-fluoro-2-[3-hydroxy-1-(methylsulfonyl)piperidin-3-yl]benzo[h][1,3]thiazolo[5,4-
f]isoquinolin-7(6H)-one;
9-fluoro-2-(1-hydroxy-1-pyridin-3-ylethyl)benzo[h][1,3]thiazolo[5,4-f]isoquinolin-
7(6H)-one;
9-fluoro-2-(1-hydroxy-1-pyridin-4-ylethyl)benzo[h][1,3]thiazolo[5,4-f]isoquinolin-
7(6H)-one;
9-fluoro-2-[1-hydroxy-1-(1-oxidopyridin-3-yl)ethyl]benzo[h][1,3]thiazolo[5,4-
f]isoquinolin-7(6H)-one;
9-fluoro-2-[1-hydroxy-1-(1-oxidopyridin-4-yl)ethyl]benzo[h][1,3]thiazolo[5,4-
f]isoquinolin-7(6H)-one;
2-{4-[(dimethylamino)methyl]piperidin-1-yl}-9-fluorobenzo[h][1,3]thiazolo[5,4-
f]isoquinolin-7(6H)-one;
9-fluoro-2-(pyridine-3-yloxy)benzo[h][1,3]thiazolo[5,4-f]isoquinolin-7(6H)-one;
9-fluoro-2-(pyridin-4-ylthio)benzo[h][1,3]thiazolo[5,4-f]isoquinolin-7(6H)-one;
9-fluoro-2-[(trans-4-hydroxycyclohexyl)amino]benzo[h][1,3]thiazolo[5,4-f]isoquinolin-
7(6H)-one;
9-fluoro-2-(4-pyrazin-2-ylpiperazin-1-yl)benzo[h][1,3]thiazolo[5,4-f]isoquinolin-7(6H)-
one;
4-(9-fluoro-7-oxo-6,7-dihydrobenzo[h][1,3]thiazolo[5,4-f]isoquinolin-2-yl)-N-
methylpiperazine-1-carboxamide;
9-fluoro-2-[4-(1H-pyrazol-3-ylcarbonyl)piperazin-1-yl]benzo[h][1,3]thiazolo[5,4-
f]isoquinolin-7(6H)-one;
9-fluoro-2-[4-(1H-pyrazol-4-ylcarbonyl)piperazin-1-yl]benzo[h][1,3]thiazolo[5,4-
f]isoquinolin-7(6H)-one;
9-fluoro-2-[4-(1H-imidazol-2-ylcarbonyl)piperazin-1-yl]benzo[h][1,3]thiazolo[5,4-
f]isoquinolin-7(6H)-one;
2-[4-(aminoacetyl)piperazin-1-yl]-9-fluorobenzo[h][1,3]thiazolo[5,4-f]isoquinolin-
7(6H)-one;
2-[4-(azetidin-3-ylcarbonyl)piperazin-1-yl]-9-fluorobenzo[h][1,3]thiazolo[5,4-
f]isoquinolin-7(6H)-one;
N-(tert-butyl)-4-(9-fluoro-7-oxo-6,7-dihydrobenzo[h][1,3]thiazolo[5,4-f]isoquinolin-2-
yl)piperazine-1-carboxamide;
9-fluoro-2-{4(2S)-2-hydroxypropyl]piperazin-1-yl}benzo[h][1,3]thiazolo[5,4-
f]isoquinolin-7(6H)-one;
3-[4-(9-fluoro-7-oxo-6,7-dihydrobenzo[h][1,3]thiazolo[5,4-f]isoquinolin-2-yl)piperazin-
1-yl]-3-oxopropanenitrile;
9-fluoro-2-[4-(methylsulfonyl)piperazin-1-yl]benzo[h][1,3]thiazolo[5,4-f]isoquinolin-
7(6H)-one;
4-(9-fluoro-7-oxo-6,7-dihydrobenzo[h][1,3]thiazolo[5,4-f]isoquinolin-2-yl)-N,N-
dimethylpiperazine-1-carboxamide;
1-(9-fluoro-7-oxo-6,7-dihydrobenzo[h][1,3]thiazolo[5,4-f]isoquinolin-2-yl)piperidine-4-
carboxylic acid;
9-fluoro-2-{4-hydroxy-4-[(methylamino)methyl]piperidin-1-
yl}benzo[h][1,3]thiazolo[5,4-f]isoquinolin-7(6H)-one;
9-fluoro-2-[4-hydroxy-4-(1H-1,2,4-triazol-1-ylmethyl)piperidin-1-
yl]benzo[h][1,3]thiazolo[5,4-f]isoquinolin-7(6H)-one;
9-fluoro-2-[4-hydroxy-4-(hydroxymethyl)piperidin-1-yl]benzo[h][1,3]thiazolo[5,4-
f]isoquinolin-7(6H)-one;
9-fluoro-2-piperidin-3-ylbenzo[h][1,3]thiazolo[5,4-f]isoquinolin-7(6H)-one;
2-(1-acetylpiperidin-3-yl)-9-fluorobenzo[h][1,3]thiazolo[5,4-f]isoquinolin-7(6H)-one;
9-fluoro-2-[1-(methylsulfonyl)piperidin-3-yl]benzo[h][1,3]thiazolo[5,4-f]isoquinolin-
7(6H)-one;
9-fluoro-2-[1-(pyridin-4-ylmethyl)piperidin-3-yl]benzo[h][1,3]thiazolo[5,4-f]isoquinolin-
7(6H)-one;
3-[3-(9-fluoro-7-oxo-6,7-dihydrobenzo[h][1,3]thiazolo[5,4-f]isoquinolin-2-yl)piperidin-
1-yl]-3-oxopropanenitrile;
9-fluoro-2-{1-[(2S)-2-hydroxypropyl]piperidin-3-yl)benzo[h][1,3]thiazolo[5,4-
f]isoquinolin-7(6H)-one;
2-{1-[(dimethylamino)acetyl]piperidin-3-yl}-9-fluorobenzo[h][1,3]thiazolo[5,4-
f]isoquinolin-7(6H)-one;
9-fluoro-2-[4-(morpholin-4-ylmethyl)piperidin-1-yl]benzo[h][1,3]thiazolo[5,4-
f]isoquinolin-7(6H)-one;
9-fluoro-2-[4-(2-morpholin-4-ylethyl)piperidin-1-yl]benzo[h][1,3]thiazolo[5,4-
f]isoquinolin-7(6H)-one;
4-(9-fluoro-7-oxo-6,7-dihydrobenzo[h][1,3]oxazolo[5,4-f]isoquinolin-2-yl)butanenitrile;
9-fluoro-2-[3-(1H-1,2,4-triazol-1-yl)propyl]benzo[h][1,3]oxazolo[5,4-f]isoquinolin-
7(6H)-one;
9-fluoro-2-{[(1R)-1-phenylethyl]amino}benzo[h][1,3]oxazolo[5,4-f]isoquinolin-7(6H)-
one;
9-fluoro-2-{[(1S)-1-phenylethyl]amino}benzo[h][1,3]oxazolo[5,4-f]isoquinolin-7(6H)-
one;
9-fluoro-2-[(2-methoxybenzyl)amino]benzo[h][1,3]oxazolo[5,44]isoquinolin-7(6H)-one;
9-fluoro-2-[(4-methoxybenzyl)amino]benzo[h][1,3]oxazolo[5,4-f]isoquinolin-7(6H)-one;
9-fluoro-2-[(3-methoxybenzyl)amino]benzo[h][1,3]oxazolo[5,4-f]isoquinolin-7(6H)-one;
9-fluoro-2-(4-oxopiperidin-1-yl)benzo[h][1,3]oxazolo[5,4-f]isoquinolin-7(6H)-one;
9-fluoro-2-(4-hydroxypiperidin-1-yl)benzo[h][1,3]oxazolo[5,4-f]isoquinolin-7(6H)-one;
9-fluoro-2-{[1-(methylsulfonyl)piperidin-4-yl]amino}benzo[h][1,3]oxazolo[5,4-
f]isoquinolin-7(6H)-one;
2-[(1-acetylpiperidin-4-yl)amino]-9-fluorobenzo[h][1,3]oxazolo[5,4-f]isoquinolin-7(6H)-
one;
9-fluoro-2-[hydroxy(pyridin-3-yl)methyl]benzo[h][1,3]oxazolo[5,4-f]isoquinolin-7(6H)-
one;
2-(4-acetylpiperazin-1-yl)-9-fluorobenzo[h][1,3]oxazolo[5,4-f]isoquinolin-7(6H)-one;
4-(9-fluoro-7-oxo-6,7-dihydrobenzo[h][1,3]oxazolo[5,4-f]isoquinolin-2-yl)piperazine-1-
carboxamide;
2-[(4-acetylpiperazin-1-yl)methyl]-9-fluorobenzo[h][1,3]oxazolo[5,4-f]isoquinolin-
7(6H)-one;
9-fluoro-2-[(trans-4-hydroxycyclohexyl)amino]benzo[h][1,3]oxazolo[5,4-f]isoquinolin-
7(6H)-one;
9-fluoro-2-(pyridin-3-yloxy)benzo[h][1,3]oxazolo[5,4-f]isoquinolin-7(6H)-one;
2-[(1-acetylpiperidin-3-yl)amino]-9-fluorobenzo[h][1,3]oxazolo[5,4-f]isoquinolin-7(6H)-
one;
9-fluoro-2-{[1-(methylsulfonyl)piperidin-3-yl]amino}benzo[h][1,3]oxazolo[5,4-
f]isoquinolin-7(6H)-one;
9-fluoro-2-[4-(1,3-thiazol-2-yl]piperazin-1-yl]benzo[h][1,3]oxazolo[5,4-f]isoquinolin-
7(6H)-one;
2-(trans-4-Hydroxycyclohexyl)imidazo[1,2-a]pyrido[4,3-c]-1,6-naphthyridin-8(7H)-one;
2-(cis-4-Hydroxycyclohexyl)imidazo[1,2-a]pyrido[4,3-c]-1,6-naphthyridin-8(7H)-one;
4-(8-Oxo-7,8-dihydroimidazo[1,2-a]pyrido[4,3-c]-1,6-naphthyridin-2-yl)piperidine-1-
carbaldehyde;
2-Piperidin-4-ylimidazo[1,2-a]pyrido[4,3-c]-1,6-naphthyridin-8(7H)-one;
2-[1-(Cyclopropylmethyl)piperidin-4-yl]imidazo[1,2-a]pyrido[4,3-c]-1,6-naphthyridin-
8(7H)-one;
2-[1-(Propyl)piperidin-4-yl]imidazo[1,2-a]pyrido[4,3-c]-1,6-naphthyridin-8(7H)-one;
2-tert-Butylimidazo[1,2-a]pyrido[4,3-c]-1,6-naphthyridin-8(7H)-one;
2-(4-Hydroxypiperidin-1-yl)-1,9-dihydro-8H-imidazo[4,5-f]-2,8-phenanthrolin-8-one;
2-(4-hydroxypiperidin-1-yl)[1,3]thiazolo[4,5-f]-2,9-phenanthrolin-8(9H)-one;
2-(isopropylamino)[1,3]thiazolo[4,5-f]-2,9-phenanthrolin-8(9H)-one;
2-[(3-methoxypropyl)amino][1,3]thiazolo[4,5-f]-2,9-phenanthrolin-8(9H)-one;
2-{[(1R)-1-(hydroxymethyl)-3-methylbutyl]amino}[1,3]thiazolo[4,5-f]-2,9-
phenanthrolin-8(9H)-one;
2-{[1-(methylsulfonyl)piperidin-4-yl)oxy}[1,3]thiazolo[4,5-f]-2,9-phenanthrolin-8(9H)-
one;
2-{[(1R)-1-(hydroxymethyl)butyl]amino}[1,3]thiazolo[4,5-f]-2,9-phenanthrolin-8(9H)-
one;
2-{[(1R)-1-(hydroxymethyl)propyl]amino}[1,3]thiazolo[4,5-f]-2,9-phenanthrolin-8(9H)-
one;
2-{[(1R)-1-(hydroxymethyl)-2-methylpropyl]amino}[1,3]thiazolo[4,5-f]-2,9-
phenanthrolin-8(9H)-one;
2-{[(1R,2R)-2-hydroxycyclohexyl]amino}[1,3]thiazolo[4,5-f]-2,9-phenanthrolin-8(9H)-
one;
2-{[(1R,2R)-2-hydroxycyclopentyl]amino}[1,3]thiazolo[4,5-f]-2,9-phenanthrolin-8(9H)-
one;
2-{[(1R)-1-(hydroxymethyl)-2,2-dimethylpropyl]amino}[1,3]thiazolo[4,5-f]-2,9-
phenanthrolin-8(9H)-one;
2-(4-{[3-(2-oxopyrrolidin-1-yl)propyl]amino}piperidin-1-yl)[1,3]thiazolo[4,5-f]-2,9-
phenanthrolin-8(9H)-one;
2-{[(1S,2S)-1-(hydroxymethyl)-2-methylbutyl]amino}[1,3]thiazolo[4,5-f]-2,9-
phenanthrolin-8(9H)-one;
2-{4-[(3-morpholin-4-ylpropyl)amino]piperidin-1-yl}[1,3]thiazolo[4,5-f]-2,9-
phenanthrolin-8(9H)-one;
2-(dimethylamino)-N-[1-(8-oxo-8,9-dihydro[1,3]thiazolo[4,5-f]-2,9-phenanthrolin-2-
yl)piperidin-4-yl]acetamide;
2-(4-hydroxypiperidin-1-yl)[1,3]thiazolo[4,5-f]-2,9-phenanthrolin-8(9H)-one;
2-(propylamino)[1,3]thiazolo[4,5-f]-2,9-phenanthrolin-8(9H)-one;
2-(4-acetylpiperazin-1-yl)[1,3]thiazolo[4,5-f]-2,9-phenanthrolin-8(9H)-one;
2-piperazin-1-yl[1,3]thiazolo[4,5-f]-2,9-phenanthrolin-8(9H)-one;
2-{4-[(2S)-2-hydroxypropyl]piperazin-1-yl}[1,3]thiazolo[4,5-f]-2,9-phenanthrolin-
8(9H)-one;
2-[4-(1H-imidazol-5-ylacetyl)piperazin-1-yl][1,3]thiazolo[4,5-f]-2,9-phenanthrolin-
8(9H)-one;
2-{4-[(2R)-2-hydroxypropyl]piperazin-1-yl}[1,3]thiazolo[4,5-f]-2,9-phenanthrolin-
8(9H)-one;
2-[4-(2-hydroxyethyl)piperazin-1-yl][1,3]thiazolo[4,5-f]-2,9-phenanthrolin-8(9H)-one;
2-{[(2R)-2-amino-3,3-dimethylbutyl]oxy}[1,3]thiazolo[4,5-f]-2,9-phenanthrolin-8(9H)-
one;
2-{[(1S)-1-(hydroxymethyl)-2,2-dimethylpropyl]amino}[1,3]thiazolo[4,5-f]-2,9-
phenanthrolin-8(9H)-one;
N-[1-(8-oxo-8,9-dihydro[1,3]thiazolo[4,5-f]-2,9-phenanthrolin-2-yl)piperidin-4-yl]-3-
pyridin-3-ylpropanamide;
2-[4-(2-morpholin-4-ylethoxy)piperidin-1-yl][1,3]thiazolo[4,5-f]-2,9-phenanthrolin-
8(9H)-one;
2-{4-[(2-morpholin-4-ylethoxy)methyl]piperidin-1-yl}[1,3]thiazolo[4,5-f]-2,9-
phenanthrolin-8(9H)-one;
2-(4-{methyl[3-(2-oxopyrrolidin-1-yl)propyl]amino}piperidin-1-yl)[1,3]thiazolo[4,5-f]-
2,9-phenanthrolin-8(9H)-one;
2-[4-(morpholin-4-ylmethyl)piperidin-1-yl][1,3]thiazolo[4,5-f]-2,9-phenanthrolin-8(9H)-
one;
2-[4-(2-morpholin-4-ylethyl)piperidin-1-yl][1,3]thiazolo[4,5]-2,9-phenanthrolin-8(9H)-
one;
2-(4-hydroxypiperidin-1-yl)[1,3]oxazolo[4,5]-2,9-phenanthrolin-8(9H)-one;
2-(4-oxopiperidin-1-yl)[1,3]oxazolo[4,5]-2,9-phenanthrolin-8(9H)-one;
2-(4-hydroxy-4-methylpiperidin-1-yl)[1,3]oxazolo[4,5-f]-2,9-phenanthrolin-8(9H)-one;
9-fluoro-2-(pyridin-4-ylmethoxy)-3,6-dihydro-7H-benzo[h]imidazo[4,5-f]isoquinolin-7-
one;
9-fluoro-2-[(pyridin-3-ylmethyl)amino]-3,6-dihydro-7H-benzo[h]imidazo[4,5-
f]isoquinolin-7-one;
9-fluoro-2-(3-piperidin-1-ylpropoxy)-1,6-dihydro-7H-benzo[h]imidazo[4,5-
f]isoquinolin-7-one;
9-fluoro-2-[(3-morpholin-4-ylpropyl)amino]-1,6-dihydro-7H-benzo[h]imidazo[4,5-
f]isoquinolin-7-one;
9-fluoro-2-4-hydroxy-4-[(2-morpholin-4-ylethoxy)methyl]piperidin-1-yl-1,6-dihydro-
7H-benzo[h]imidazo[4,5-f]isoquinolin-7-one;
9-fluoro-2-(pyridin-3-yloxy)-3,6-dihydro-7H-benzo[h]imidazo[4,5-f]isoquinolin-7-one;
9-fluoro-2-isopropoxy-3,6-dihydro-7H-benzo[h]imidazo[4,5-f]isoquinolin-7-one;
9-fluoro-2-(pyridin-3-ylmethoxy)-3,6-dihydro-7H-benzo[h]imidazo[4,5-f]isoquinolin-7-
one;
9-fluoro-2-(4-oxopyridin-1(4H)-yl)-3,6-dihydro-7H-benzo[h]imidazo[4,5-f]isoquinolin-
7-one;
9-fluoro-2-[(cis-4-hydroxycyclohexyl)oxy]-3,6-dihydro-7H-benzo[h]imidazo[4,5-
f]isoquinolin-7-one;
9-fluoro-2-[(trans-4-hydroxycyclohexyl)oxy]-3,6-dihydro-7H-benzo[h]imidazo[4,5-
f]isoquinolin-7-one;
9-fluoro-2-(pyridin-2-ylmethoxy)-3,6-dihydro-7H-benzo[h]imidazo[4,5-f]isoquinolin-7-
one;
9-fluoro-2-[(trans-4-hydroxycyclohexyl)amino]-3,6-dihydro-7H-benzo[h]imidazo[4,5-
f]isoquinolin-7-one;
9-fluoro-2-[(pyridin-4-ylmethyl)amino]-3,6-dihydro-7H-benzo[h]imidazo[4,5-
f]isoquinolin-7-one;
9-fluoro-2-[(1-oxidopyridin-2-yl)methoxy]-3,6-dihydro-7H-benzo[h]imidazo[4,5-
f]isoquinolin-7-one;
9-fluoro-2-[(1-oxidopyridin-3-yl)methoxy]-3,6-dihydro-7H-benzo[h]imidazo[4,5-
f]isoquinolin-7-one;
9-fluoro-2-[(1R,3R)-3-hydroxycyclohexyl]oxy-3,6-dihydro-7H-benzo[h]imidazo[4,5-
f]isoquinolin-7-one;
cis- and trans-9-fluoro-2-[3-hydroxycyclohexyl]oxy-3,6-dihydro-7H-
benzo[h]imidazo[4,5-f]isoquinolin-7-one;
9-fluoro-2-[1-(methylsulfonyl)piperidin-4-yl]oxy-3,6-dihydro-7H-benzo[h]imidazo[4,5-
f]isoquinolin-7-one;
2-[(1-acetylpiperidin-4-yl)oxy]-9-fluoro-3,6-dihydro-7H-benzo[h]imidazo[4,5-
f]isoquinolin-7-one;
3-4-[(9-fluoro-7-oxo-6,7-dihydro-3H-benzo[h]imidazo[4,5-f]isoquinolin-2-
yl)oxy]piperidin-1-yl-3-oxopropanenitrile;
9-fluoro-2-[(trans-4-hydroxycyclohexyl)(methyl)amino]-3,6-dihydro-7H-
benzo[h]imidazo[4,5-f]isoquinolin-7-one;
9-fluoro-2-(3-morpholin-4-ylpropoxy)-1,6-dihydro-7H-benzo[h]imidazo[4,5-
f]isoquinolin-7-one;
9-fluoro-2-[3-(2-oxopyrrolidin-1-yl)propyl]amino-1,6-dihydro-7H-benzo[h]imidazo[4,5-
f]isoquinolin-7-one;
2-(4-{[bis(2-methoxyethyl)amino]methyl}piperidin-1-yl)-9-fluoro-1,6-dihydro-7H-
benzo[h]imidazo[4,5-f]isoquinolin-7-one;
9-fluoro-2-{4-[(2-morpholin-4-ylethoxy)methyl]piperidin-1-yl}-1,6-dihydro-7H-
benzo[h]imidazo[4,5-f]isoquinolin-7-one;
2-[(cyclopropylmethyl)(3-morpholin-4-ylpropyl)amino]-9-fluoro-1,6-dihydro-7H-
benzo[h]imidazo[4,5-f]isoquinolin-7-one;
9-fluoro-2-4-hydroxy-4-[(3-piperidin-1-ylpropoxy)methyl]piperidin-1-yl-1,6-dihydro-
7H-benzo[h]imidazo[4,5-f]isoquinolin-7-one;
9-fluoro-2-4-hydroxy-4-[(3-morpholin-4-ylpropoxy)methyl]piperidin-1-yl-1,6-dihydro-
7H-benzo[h]imidazo[4,5-f]isoquinolin-7-one;
10-fluoro-2-[4-(hydroxyimino)cyclohexyl]benzo[c]imidazo[1,2-a]-1,6-naphthyridin-
8(7H)-one;
9-fluoro-2-4-[(2-morpholin-4-ylethoxy)imino]piperidin-1-yl-1,6-dihydro-7H-
benzo[h]imidazo[4,5-f]isoquinolin-7-one;
4-ethyl-4-(9-fluoro-7-hydroxy-3H-benzo[f]imidazo[4,5-h]phthalazin-2-yl)hexanenitrile;
4-ethyl-4-(9-fluoro-7-oxo-6,7-dihydro-3H-benzo[f]imidazo[4,5-h]phthalazin-2-
yl)hexanoic acid;
4-ethyl-4-(9-fluoro-7-hydroxy-3H-benzo[f]imidazo[4,5-h]phthalazin-2-yl)hexanamide;
methyl2-(9-fluoro-7-hydroxy-3H-benzo[f]imidazo[4,5-h]phthalazin-2-yl)-2-
methylpropanoate;
2-(9-fluoro-7-hydroxy-3H-benzo[f]imidazo[4,5-h]phthalazin-2-yl)-2-methylpropanoic
acid;
9-fluoro-2-(2-hydroxy-1,1-dimethylethyl)-3H-benzo[f]imidazo[4,5-h]phthalazin-7-ol;
4-(9-fluoro-7-oxo-6,7-dihydro-3H-benzo[f]imidazo[4,5-h]phthalazin-2-yl)-4-
methylpentanenitrile;
(2E)-4-(9-fluoro-7-ozo-6,7-dihydro-3H-benzo[f]imidazo[4,5-h]phthalazin-2-yl)-4-
methylpent-2-enenitrile;
9-fluoro-2-[4-(methylthio)phenyl]-3,6-dihydro-7H-benzo[f]imidazo[4,5-h]phthalazin-7-
one;
9-fluoro-2-[4-(methylsulfinyl)phenyl]-3,6-dihydro-7H-benzo[f]imidazo[4,5-
h]phthalazin-7-one;
4-(9-fluoro-7-hydroxy-3H-benzo[f]imidazo[4,5-h]phthalazin-2-yl)-4-
methylpentanamide;
3-(9-fluoro-7-hydroxy-3H-benzo[f]imidazo[4,5-h]phthalazin-2-yl)propanenitrile;
9-fluoro-2-(2-pyridin-2-ylethyl)-3H-benzo[f]imidazo[4,5-h]phthalazin-7-ol;
9-fluoro-2-(pyridin-4-ylmethyl)-3,6-dihydro-7H-benzo[f]imidazo[4,5-h]phthalazin-7-
one;
9-fluoro-2-(pyridin-3-ylmethyl)-3,6-dihydro-7H-benzo[f]imidazo[4,5-h]phthalazin-7-
one;
2-[4-(benzyloxy)cyclohexyl]-9-fluoro-3,6-dihydro-7H-benzo[t]imidazo[4,5-h]phthalazin-
7-one;
9-fluoro-2-(4-hydroxycyclohexyl)-3,6-dihydro-7H-benzo[f]imidazo[4,5-h]phthalazin-7-
one;
methyl2-(9-fluoro-7-hydroxybenzo[f][1,3]oxazolo[5,4-h]phthalazin-2-yl)-2-
methylpropanoate;
benzyl[4-(9-fluoro-7-hydroxybenzo[f][1,3]oxazolo[5,4-h]phthalazin-2-yl)-4-
methylpentyl]carbamate;
2-(4-amino-1,1-dimethylbutyl)-9-fluorobenzo[f][1,3]oxazolo[5,4-h]phthalazin-7-ol;
2-(9-fluoro-7-hydroxybenzo[f][1,3]oxazolo[5,4-h]phthalazin-2-yl)-2-methylpropanoic
acid;
2-tert-butyl-1,9-dihydro-8H-imidazo[4,5-f]-2,8-phenanthrolin-8-one;
2-tert-butyl-3,6-dihydro-7H-imidazo[4,5-f]-1,9-phenanthrolin-7-one;
2-cyclohexyl-1,9-dihydro-8H-imidazo[4,5-f]-2,8-phenanthrolin-8-one;
3-(8-oxo-8,9-dihydro-1H-imidazo[4,5-f]-2,8-phenanthrolin-2-yl)propanenitrile;
2-(trans-4-hydroxycyclohexyl)-1,9-dihydro-8H-imidazo[4,5-f]-2,8-phenanthrolin-8-one;
566]2-[1-(methylsulfonyl)piperidin-4-yl]-1,9-dihydro-8H-imidazo[4,5-f]-2,8-
phenanthrolin-8-one;
3-oxo-3-[4-(8-oxo-8,9-dihydro-1H-imidazo[4,5-f]-2,8-phenanthrolin-2-yl)piperidin-1-
yl]propanenitrile;
3-methyl-3-(8-oxo-8,9-dihydro-1H-imidazo[4,5-f]-2,8-phenanthrolin-2-yl)butanenitrile;
2-tert-butyl-1,9-dihydro-8H-imidazo[4,5-f]-2,7-phenanthrolin-8-one;
2-cyclohexyl-1,9-dihydro-8H-imidazo[4,5-f]-2,7-phenanthrolin-8-one;
2-cyclopentyl-1,9-dihydro-8H-imidazo[4,5-f]-2,7-phenanthrolin-8-one;
2-tert-butyl-1,9-dihydro-8H-imidazo[4,5-f]-2,8-phenanthrolin-8-one 5-oxide;
3-methyl-3-(5-oxido-8-oxo-8,9-dihydro-1H-imidazo[4,5-f]-2,8-phenanthrolin-2-
yl)butanenitrile;
3-{3-[methyl(1H-pyrrolo[2,3-b]pyridin-4-yl)amino]piperidin-1-yl}-3-oxopropanenitrile;
3-{(3R,4R)-4-methyl-3-[methyl(1H-pyrrolo[2,3-b]pyridin-4-yl)amino]piperidin-1-yl}-3-
oxopropanenitrile;
N-methyl-N-[(3R,4R)-4-methyl-1-(phenylsulfonyl)piperidin-3-yl]-N-(1H-pyrrolo[2,3-
b]pyridin-4-yl)-amine;
N-[(3R,4R)-1-(methoxyacetyl)-4-methylpiperidin-3-yl]-N-methyl-N-(1H-pyrrolo[2,3-
b]pyridin-4-yl)-amine;
(3R,4R)-4-methyl-3-[methyl(1H-pyrrolo[2,3-b]pyridin-4-yl)amino]-N-phenylpiperidine-
1-carboxamide;
(3R,4R)-N-benzyl-4-methyl-3-[methyl(1H-pyrrolo[2,3-b]pyridin-4-yl)amino]piperidine-
1-carboxamide;
(3R,4R)-N-ethyl-4-methyl-3-[methyl(1H-pyrrolo[2,3-b]pyridin-4-yl)amino]piperidine-1-
carboxamide;
(3R,4R)-N-isopropyl-4-methyl-3-[methyl(1H-pyrrolo[2,3-b]pyridin-4-y-
l)amino]piperidine-1-carboxamide;
N-[(3R,4R)-1-isobutyryl-4-methylpiperidin-3-yl]-N-methyl-N-(1H-pyrrolo[2,3-
b]pyridin-4-yl)-amine;
N-methyl-N-[(3R,4R)-4-methyl-1-(morpholin-4-ylcarbonyl)piperidin-3-yl]-N-(1H-
pyrrolo[2,3-b]pyridin-4-yl)-amine;
N-[(3R,4R)-1-acetyl-4-methylpiperidin-3-yl]-N-methyl-N-(1H-pyrrolo[2,3-b]pyridin-4-
yl)-amine;
N-methyl-N-[(3R,4R)-4-methyl-1-(3-methylbutanoyl)piperidin-3-yl]-N-(1H-pyrrolo[2,3-
b]pyridin-4-yl)-amine;
N-[(3R,4R)-1-benzoyl-4-methylpiperidin-3-yl]-N-methyl-N-(1H-pyrrolo[2,3-b]pyridin-
4-yl)-amine;
(3R,4R)-N,N,4-trimethyl-3-[methyl(1H-pyrrolo[2,3-b]pyridin-4-yl)amino]piperidine-1-
carboxamide;
4-({(3R,4R)-4-methyl-3-[methyl(1H-pyrrolo[2,3-b]pyridin-4-yl)amino]piperidin-1-
yl}carbonyl)benzonitrile;
N-[(3R,4R)-1-(cyclopropylearbonyl)-4-methylpiperidin-3-yl]-N-methyl-N-(1H-
pyrrolo[2,3-b]pyridin-4-yl)-amine;
N-[(3R,4R)-1-isonicotinoyl-4-methylpiperidin-3-yl]-N-methyl-N-(1H-pyrrolo[2,3-
b]pyridin-4-yl)-amine;
N-{(3R,4R)-1-[(1-acetylpiperidin-4-yl)carbonyl]-4-methylpiperidin-3-yl}-N-methyl-N-
(1H-pyrrolo[2,3-b]pyridin-4-yl)-amine;
Phenyl(3R,4R)-4-methyl-3-[methyl(1H-pyrrolo[2,3-b]pyridin-4-yl)amino]piperidine-1-
carboxylate;
Methyl(3R,4R)-4-methyl-3-[methyl(1H-pyrrolo[2,3-b]pyridin-4-yl)amino]piperidine-1-
carboxylate;
N-methyl-N-[(3R,4R)-4-methyl-1-(trifluoroacetyl)piperidin-3-yl]-N-(1H-pyrrolo[2,3-
b]pyridin-4-yl)-amine;
N-[(3R,4R)-1-(2-furoyl)-4-methylpiperidin-3-yl]-N-methyl-N-(1H-pyrrolo[2,3-
b]pyridin-4-yl)-amine;
(3R,4R)-N-(4-cyanophenyl)-4-methyl-3-[methyl(1H-pyrrolo[2,3-b]pyridin-4-
yl)amino]piperidine-1-carboxamide;
(3R,4R)-N-(3-cyanophenyl)-4-methyl-3-[methyl(1H-pyrrolo[2,3-b]pyridin-4-
yl)amino]piperidine-1-carboxamide;
(3R,4R)-4-methyl-3-[methyl(1H-pyrrolo[2,3-b]pyridin-4-yl)amino]-N-(2-
phenylethyl)piperidine-1-carboxamide;
(3R,4R)-N-(2-furylmethyl)-4-methyl-3-[methyl(1H-pyrrolo[2,3-b]pyridin-4-
yl)amino]piperidine-1-carboxamide;
N-methyl-N-[(3R,4R)-4-methyl-1-(propylsulfonyl)piperidin-3-yl]-N-(1H-pyrrolo[2,3-
b]pyridin-4-yl)-amine;
N-[(3R,4R)-1-(isopropylsulfonyl)-4-methylpiperidin-3-yl]-N-methyl-N-(1H-pyrrolo[2,3-
b]pyridin-4-yl)-amine;
4-({(3R,4R)-4-methyl-3-[methyl(1H-pyrrolo[2,3-b]pyridin-4-yl)amino]piperidin-1-
yl}sulfonyl)benzonitrile;
2-({(3R,4R)-4-methyl-3-[methyl(1H-pyrrolo[2,3-b]pyridin-4-yl)amino]piperidin-1-
yl}sulfonyl)benzonitrile;
N-methyl-N-[(3R,4R)-4-methyl-1-(methylsulfonyl)piperidin-3-yl]-N-(1H-pyrrolo[2,3-
b]pyridin-4-yl)-amine;
N-methyl-N-{(3R,4R)-4-methyl-1-[(trifluoromethyl)sulfonyl]piperidin-3-yl}-N-(1H-
pyrrolo[2,3-b]pyridin-4-yl)-amine;
N-methyl-N-[(3R,4R)-4-methyl-1-(pyridin-3-ylsulfonyl)piperidin-3-yl]-N-(1H-
pyrrolo[2,3-b]pyridin-4-yl)-amine;
2-fluoro-5-({(3R,4R)-4-methyl-3-[methyl(1H-pyrrolo[2,3-b]pyridin-4-
yl)amino]piperidin-1-yl}sulfonyl)benzonitrile;
N-methyl-N-[(3R,4R)-4-methyl-1-(3-pyridin-3-ylpropanoyl)piperidin-3-yl]-N-(1H-
pyrrolo[2,3-b]pyridin-4-yl)-amine;
N-methyl-N-[(3R,4R)-4-methyl-1-(3,3,3-trifluoropropanoyl)piperidin-3-yl]-N-(1H-
pyrrolo[2,3-b]pyridin-4-yl)-amine;
N-methyl-N-[(3R,4R)-4-methyl-1-(tetrahydrofuran-2-ylcarbonyl)piperidin-3-yl)-N-(1H-
pyrrolo[2,3-b]pyridin-4-yl)-amine;
(2R)-1-{(3R,4R)-4-methyl-3-[methyl(1H-pyrrolo[2,3-b]pyridin-4-yl)amino]piperidin-1-
yl}-1-oxopropan-2-ol;
(2S)-1-{(3R,4R)-4-methyl-3-[methyl(1H-pyrrolo[2,3-b]pyridin-4-yl)amino]piperidin-1-
yl}-1-oxopropan-2-ol;
N-methyl-N-[(3R,4R)-4-methyl-1-(3-phenylpropanoyl)piperidin-3-yl]-N-(1H-
pyrrolo[2,3-b]pyridin-4-yl)-amine;
(3R,4R)-N-(4-cyanophenyl)-4-methyl-3-[methyl(1H-pyrrolo[2,3-b]pyridin-4-
yl)amino]piperidine-1-carbothioamide;
N-methyl-N-{(3R,4R)-4-methyl-1-[(5-methylisoxazol-4-yl)sulfonyl]piperidin-3-yl}-N-
(1H-pyrrolo[2,3-b]pyridin-4-yl)-amine;
N-methyl-N-{(3R,4R)-4-methyl-1-[(1-methyl-1H-imidazol-4-yl)sulfonyl]piperidin-3-
yl}-N-(1H-pyrrolo[2,3-b]pyridin-4-yl)-amine;
N-{(3R,4R)-1-[(3,5-dimethylisoxazol-4-yl)carbonyl]-4-methylpiperidin-3-yl}-N-methyl-
N-(1H-pyrrolo[2,3-b]pyridin-4-yl)-amine;
(3R,4R)-4-methyl-3-[methyl(1H-pyrrolo[2,3-b]pyridin-4-yl)amino)-N-2-
thienylpiperidine-1-carboxamide;
N-[(3R,4R)-1-(isoxazol-5-ylcarbonyl)-4-methylpiperidin-3-yl]-N-methyl-N-(1H-
pyrrolo[2,3-b]pyridin-4-yl)-amine;
N-{(3R,4R)-1-[(1,2-dimethyl-1-H-imidazol-4-yl)sulfonyl]-4-methylpiperidin-3-yl}-N-
methyl-N-(1H-pyrrolo[2,3-b]pyridin-4-yl)-amine;
N-[4-methyl-5-({(3R,4R)-4-methyl-3-[methyl(1H-pyrrolo[2,3-b]pyridin-4-
yl)amino]piperidin-1-yl}sulfonyl)-1,3-thiazol-2-yl]acetamide;
N-{(3R,4R)-1-[(2,4-dimethyl-1,3-thiazol-5-yl)sulfonyl]-4-methylpiperidin-3-yl}-N-
methyl-N-(1H-pyrrolo[2,3-b]pyridin-4-yl)-amine;
N-methyl-N-{(3R,4R)-4-methyl-1-[(1,3,5-trimethyl-1H-pyrazol-4-yl)sulfonyl]piperidin-
3-yl}-N-(1H-pyrrolo[2,3-b]pyridin-4-yl)-amine;
N-{(3R,4R)-1-[(3,5-dimethylisoxazol-4-yl)sulfonyl]-4-methylpiperidin-3-yl}-N-methyl-
N-(1H-pyrrolo[2,3-b]pyridin-4-yl)-amine;
N-methyl-N-{(3R,4R)-4-methyl-1-[(pyridin-4-ylmethyl)sulfonyl]piperidin-3-yl}-N-(1H-
pyrrolo[2,3-b]pyridin-4-yl)-amine;
N-methyl-N-{(3R,4R)-4-methyl-1-[(pyridin-3-ylmethyl)sulfonyl]piperidin-3-yl}-N-(1H-
pyrrolo[2,3-b]pyridin-4-yl)-amine;
N-methyl-N-{(3R,4R)-4-methyl-1-[(pyridin-2-ylmethyl)sulfonyl]piperidin-3-yl{-N-(1H-
pyrrolo[2,3-b]pyridin-4-yl)-amine;
4-({(3R,4R)-4-methyl-3-[methyl(1H-pyrrolo[2,3-b]pyridin-4-yl)amino]piperidin-1-
yl}sulfonyl)benzonitrile;
(3R,4R)-N-(4-cyanophenyl)-N,4-dimethyl-3-[methyl(1H-pyrrolo[2,3-b]pyridin-4-
yl)amino]piperidine-1-carboxamide;
(3R,4R)-N-(4-cyanophenyl)-N-ethyl-4-methyl-3-[methyl(1H-pyrrolo[2,3-b]pyridin-4-
yl)amino]piperidine-1-carboxamide;
(3R,4R)-4-methyl-3-[methyl(1H-pyrrolo[2,3-b]pyridin-4-yl)amino]-N-1,3-thiazol-2-
ylpiperidine-1-carboxamide;
(3R,4R)-4-methyl-N-(3-methylisoxazol-5-yl)-3-[methyl(1H-pyrrolo[2,3-b]pyridin-4-
yl)amino]piperidine-1-carboxamide;
3-chloro-4-({(3R,4R)-4-methyl-3-[methyl(1H-pyrrolo[2,3-b]pyridin-4-
yl)amino]piperidin-1-yl}sulfonyl)benzonitrile;
(3R,4R)-4-methyl-N-(5-methylisoxazol-3-yl)-3-[methyl(1H-pyrrolo[2,3-b]pyridin-4-
yl)amino]piperidine-1-carboxamide;
(3R,4R)-N-isoxazol-3-yl-4-methyl-3-[methyl(1H-pyrrolo[2,3-b]pyridin-4-
yl)amino]piperidine-1-carboxamide;
N-methyl-N-{(3R,4R)-4-methyl-1-[(5-pyridin-3-yl-2-thienyl)sulfonyl]piperidin-3-yl}-N-
(1H-pyrrolo[2,3-b]pyridin-4-yl)-amine;
(3R,4R)-N-(3-cyano-2-thienyl)-4-methyl-3-[methyl(1H-pyrrolo[2,3-b]pyridin-4-
yl)amino]piperidine-1-carboxamide;
(3R,4R)-N-1,3-benzothiazol-2-yl-4-methyl-3-[methyl(1H-pyrrolo[2,3-b]pyridin-4-
yl)amino]piperidine-1-carboxamide;
N-[(3R,4R)-1-(2,3-dihydro-1H-indol-1-ylcarbonyl)-4-methylpiperidin-3-yl]-N-methyl-
N-(1H-pyrrolo[2,3-b]pyridin-4-yl)-amine;
N-methyl-N-{(3R,4R)-4-methyl-1-[(methylthio)acetyl]piperidin-3-yl}-N-(1H-
pyrrolo[2,3-b]pyridin-4-yl)-amine;
(3R,4R)-N-(4,5-dihydro-1,3-thiazol-2-yl)-4-methyl-3-[methyl(1H-pyrrolo[2,3-b]pyridin-
4-yl)amino]piperidine-1-carboxamide;
(3R,4R)-4-methyl-3-[methyl(1H-pyrrolo[2,3-b]pyridin-4-yl)amino]-N-(1,3-thiazol-2-
ylmethyl)piperidine-1-carboxamide;
1-({(3R,4R)-4-methyl-3-[methyl(1H-pyrrolo[2,3-b]pyridin-4-yl)amino]piperidin-1-
yl}carbonyl)piperidine-4-carbonitrile;
1-({(3R,4R)-4-methyl-3-[methyl(1H-pyrrolo[2,3-b]pyridin-4-yl)amino]piperidin-1-
yl}carbonyl)piperidine-3-carbonitrile;
1-({(3R,4R)-4-methyl-3-[methyl(1H-pyrrolo[2,3-b]pyridin-4-yl)amino]piperidin-1-
yl}carbonyl)pyrrolidine-3-carbonitrile;
(3R,4R)-4-methyl-3-[methyl(1H-pyrrolo[2,3-b]pyridin-4-yl)amino]-N-(3-
thienylmethyl)piperidine-1-carboxamide;
(3R,4R)-N-(2-benzothien-1-ylmethyl)-4-methyl-3-[methyl(1H-pyrrolo[2,3-b]pyridin-4-
yl)amino]piperidine-1-carboxamide;
(3R,4R)-N-(1,3-benzothiazol-2-ylmethyl)-4-methyl-3-[methyl(1H-pyrrolo[2,3-b]pyridin-
4-yl)amino]piperidine-1-carboxamide;
N-[(3R,4R)-1-(3-furylacetyl)-4-methylpiperidin-3-yl]-N-methyl-N-(1H-pyrrolo[2,3-
b]pyridin-4-yl)-amine;
3-(2-{(3R,4R)-4-methyl-3-[methyl(1H-pyrrolo[2,3-b]pyridin-4-yl)amino]piperidin-1-yl}-
2-oxoethyl)-1,3-thiazolidine-2,4-dione;
3-(2-{(3R,4R)-4-methyl-3-[methyl(1H-pyrrolo[2,3-b]pyridin-4-yl)amino]piperidin-1-yl}-
2-oxoethyl)-1,3-benzothiazol-2(3H)-one;
(3R,4R)-N-[5-(cyanomethyl)-4,5-dihydro,-1,3-thiazol-2-yl]-3-[methyl-(1H-pyrrolo[2,3-
b]pyridin-4-yl)amino]-4-methyl-piperidine-1-carboxamide;
(3S)-1-({(3R,4R)-4-methyl-3-[methyl(1H-pyrrolo[2,3-b]pyridin-4-yl)amino]piperidin-1-
yl}carbonyl)pyrrolidine-3-carbonitrile;
(3R)-1-({(3R,4R)-4-methyl-3-[methyl(1H-pyrrolo[2,3-b]pyridin-4-yl)amino]piperidin-1-
yl}carbonyl)pyrrolidine-3-carbonitrile;
1-({(3R,4R)-4-methyl-3-[methyl(1H-pyrrolo[2,3-b]pyridin-4-yl)amino]piperidin-1-
yl}carbonyl)-4-phenylpiperidine-4-carbonitrile;
N-methyl-N-((3R,4R)-4-methyl-1-{[3-(trifluoromethyl)pyrrolidin-1-
yl]carbonyl}piperidin-3-yl)-N-(1H-pyrrolo[2,3-b]pyridin-4-yl)-amine;
1-({(3R,4R)-4-methyl-3-[methyl(1H-pyrrolo[2,3-b]pyridin-4-yl)amino]piperidin-1-
yl}carbonyl)azetidine-3-carbonitrile;
4-methyl-1-({(3R,4R)-4-methyl-3-[methyl(1H-pyrrolo[2,3-b]pyridin-4-
yl)amino]piperidin-1-yl}carbonyl)pyrrolidine-3-carbonitrile;
1-({(3R,4R)-4-methyl-3-[methyl(1H-pyrrolo[2,3-b]pyridin-4-yl)amino]piperidin-1-
yl}carbonyl)pyrrolidine-3,4-dicarbonitrile;
3-methyl-1-({(3R,4R)-4-methyl-3-[methyl(1H-pyrrolo[2,3-b]pyridin-4-
yl)amino]piperidin-1-yl}carbonyl)pyrrolidine-3-carbonitrile;
(3R,4R)-N-(2-cyanoethyl)-4-methyl-3-[methyl(1H-pyrrolo[2,3-b]pyridin-4-
yl)amino]piperidine-1-carboxamide;
1770]4-methoxy-1-({(3R,4R)-4-methyl-3-[methyl(1H-pyrrolo[2,3-b]pyridin-4-
yl)amino]piperidin-1-yl}carbonyl)pyrrolidine-3-carbonitrile;
N-{(3R,4R)-1-[(2R)-2-aminopropanoyl]-4-methylpiperidin-3-yl}-N-methyl-N-(1H-
pyrrolo[2,3-b]pyridin-4-yl)-amine;
N-[(3R,4R)-1-(aminoacetyl)-4-methylpiperidin-3-yl]-N-methyl-N-(1H-pyrrolo[2,3-
b]pyridin-4-yl)-amine;
1-(2-{(3R,4R)-4-methyl-3-[methyl(1H-pyrrolo[2,3-b]pyridin-4-yl)amino]piperidin-1-yl}-
2-oxoethyl)piperidine-4-carbonitrile;
N-methyl-N-[(3R,4R)-4-methyl-1-(1,3-thiazol-4-ylcarbonyl)piperidin-3-yl]-N-(1H-
pyrrolo[2,3-b]pyridin-4-yl)-amine bis(trifluoroacetate);
4-(2-2S-{[methyl-(1H-pyrrolo[2,3-b]pyridin-4-yl)-amino]-methyl}-pyrrolidin-1-yl-
sulfonyl)-benzonitrile trifluoroacetate;
N-[(1-methanesulfonyl-2S-pyrrolidin-2-yl)-methyl]-N-methyl-N-(1H-pyrrolo[2,3-
b]pyridin-4-yl)-amine trifluoroacetate;
3-((2S)-2-{[methyl(1H-pyrrolo[2,3-b]pyridin-4-yl)amino]methyl}pyrrolidin-1-yl)-3-
oxopropanenitrile trifluoroacetate;
Methyl 3-[({(3R,4R)-4-methyl-3-[methyl(1H-pyrrolo[2,3-b]pyridin-4-
yl)amino]piperidine-1-yl}carbonyl)-amino]benzoate;
(3R,4R)-N-(4-trifluoromethoxyphenyl)-4-methyl-3-[methyl-(1H-pyrrolo[2,3-b]pyridin-4-
yl)amino]piperidine-1-carboxamide;
(3R,4R)-N-(4-fluorophenyl)-4-methyl-3-[methyl-(1H-pyrrolo[2,3-b]pyridin-4-
yl)amino]piperidine-1-carboxamide;
(3R,4R)-N-(3-fluorophenyl)-4-methyl-3-[methyl-(1H-pyrrolo[2,3-b]pyridin-4-
yl)amino]piperidine-1-carboxamide;
(3R,4R)-N-(2-fluorophenyl)-4-methyl-3-[methyl-(1H-pyrrolo[2,3-b]pyridin-4-
yl)amino]piperidine-1-carboxamide;
(3R,4R)-N-(4-trifluoromethylphenyl)-4-methyl-3-[methyl-(1H-pyrrolo[2,3-b]pyridin-4-
yl)amino]piperidine-1-carboxamide;
(3R,4R)-N-(2-methoxyphenyl)-4-methyl-3-[methyl(1H-pyrrolo[2,3-b]pyridin-4-
yl)amino]piperidine-1-carboxamide;
(3R,4R)-4-methyl-N-(4-methylphenyl)-3-[methyl(1H-pyrrolo[2,3-b]pyridin-4-yl)-
amino]piperidine-1-carboxamide;
N-methyl-N-{(3R,4R)-4-methyl-1-[4-(pyridin-2-yloxy)phenyl]sulfonyl-piperidin-3-yl}-
N-(1H-pyrrolo[2,3-b]pyridin-4-yl)-amine;
N-methyl-N-{(3R,4R)-4-methyl-1-[4-(1,3-oxazol-5-yl)phenyl]sulfonyl-piperidin-3-yl}-
N-(1H-pyrrolo[2,3-b]pyridin-4-yl)-amine;
N-methyl-N-{(3R,4R)-4-methyl-[5-(1,3-oxazol-5-yl)thienyl]sulfonyl-piperidin-3-yl}-N-
(1H-pyrrolo[2,3-b]pyridin-4-yl)-amine;
N-methyl-N-{(3R,4R)-4-methyl-1-[(6-phenoxy-pyridin-3-yl)sulfonyl]piperidin-3-yl}-
(1H-pyrrolo[2,3-b]pyridin-4-yl)amine;
N-{(3R,4R)-1-[(2,6-dichlorophenyl)sulfonyl]-4-methylpiperidin-3-yl}-N-methyl-(1H-
pyrrolo[2,3-b]pyridin-4-yl)-amine;
N-{(3R,4R)-1-[(4-fluorophenyl)sulfonyl]-4-methylpiperidin-3-yl}-N-methyl-(1H-
pyrrolo[2,3-b]pyridin-4-yl)-amine;
N-{(3R,4R)-1-[(3-fluorophenyl)sulfonyl]-4-methylpiperidin-3-yl}-N-methyl-(1H-
pyrrolo[2,3-b]pyridin-4-yl)-amine;
N-{(3R,4R)-1-[(2-fluorophenyl)sulfonyl]-4-methylpiperidin-3-yl}-N-methyl-(1H-
pyrrolo[2,3-b]pyridin-4-yl)-amine;
N-{(3R,4R)-4-methyl-1-[4-(trifluoromethyl)phenyl]sulfonyl-piperidin-3-yl}-N-(1H-
pyrrolo[2,3-b]pyridin-4-yl)-amine;
N-{(3R,4R)-4-methyl-[3-(trifluoromethyl)phenyl]sulfonyl-piperidin-3-yl}-N-(1H-
pyrrolo[2,3-b]pyridin-4-yl)-amine;
N-{(3R,4R)-4-methyl-[2-(trifluoromethyl)phenyl]sulfonyl-piperidin-3-yl}-N-(1H-
pyrrolo[2,3-b]pyridin-4-yl)-amine;
N-{(3R,4R)-1-[(4-methoxyphenyl)sulfonyl]-4-methylpiperidin-3-yl}-N-methyl-N-(1H-
pyrrolo[2,3-b]pyridin-4-yl)-amine;
N-{(3R,4R)-1-[(3-methoxyphenyl)sulfonyl]-4-methylpiperidin-3-yl}-N-methyl-N-(1H-
pyrrolo[2,3-b]pyridin-4-yl)-amine;
N-methyl-N-{(3R,4R)-4-methyl-1-[(4-methylphenyl)sulfonyl]piperidin-3-yl}-N-(1H-
pyrrolo[2,3-b]pyridin-4-yl)-amine;
N-methyl-N-{(3R,4R)-4-methyl-1-[(3-methylphenyl)sulfonyl]piperidin-3-yl}-N-(1H-
pyrrolo[2,3-b]pyridin-4-yl)-amine;
N-methyl-N-{(3R,4R)-4-methyl-1-[(2-methylphenyl)sulfonyl]piperidin-3-yl}-N-(1H-
pyrrolo[2,3-b]pyridin-4-yl)-amine;
N-{(3R,4R)-1-[(4-chlorophenyl)sulfonyl]-4-methylpiperidin-3-yl}-N-methyl-N-(1H-
pyrrolo[2,3-b]pyridin-4-yl)-amine;
N-{(3R,4R)-1-[(3-chlorophenyl)sulfonyl]-4-methylpiperidin-3-yl}-N-methyl-N-(1H-
pyrrolo[2,3-b]pyridin-4-yl)-amine;
N-{(3R,4R)-1-[(2-chlorophenyl)sulfonyl]-4-methylpiperidin-3-yl}-N-methyl-N-(1H-
pyrrolo[2,3-b]pyridin-4-yl)-amine
4-(3,8-dihydroimidazo[4,5-d]dipyrido[2,3-b:4′,3′-f]azepin-2-yl)-4-ethylhexanenitrile;
2-[4-(hydroxymethyl)-cyclohexyl]-imidazo[4,5-d]dipyrido[2,3-b:4′,3′-f]azepin-3(8H)-ol;
[4-(3,8-dihydroimidazo[4,5-d]dipyrido[2,3-b:4′,3′-f]azepin-2-yl)cyclohexyl]methanol;
[4-(3-hydroxy-3,8-dihydroimidazo[4,5-d]dipyrido[2,3-b:4′,3′-f]azepin-2-
yl)cyclohexyl]acetonitrile;
[4-(3,8-dihydroimidazo[4,5-d]dipyrido[2,3-b:4′,3′-f]azepin-2-yl)cyclohexyl]acetonitrile;
914]3-(3,8-dihydroimidazo[4,5-d]dipyrido[2,3-b:4′,3′-f]azepin-2-yl)-3-
methylbutanenitrile;
3-(3,8-dihydroimidazo[4,5-d]dipyrido[2,3-b:4′,3′-f]azepin-2-yl)-3-ethylpentanenitrile;
[3-(3,8-dihydroimidazo[4,5-d]dipyrido[2,3-b:4′,3′-f]azepin-2-yl)cyclopentyl]methanol;
3-(3,8-dihydroimidazo[4,5-d]dipyrido[2,3-b:4′,3′-f]azepin-2-yl)cyclopentanecarbonitrile;
2-(3,5-dichloropyridin-4-yl)-3,8-dihydroimidazo[4,5-d]dipyrido[2,3-b:4′,3′-f]azepine;
2-(2,6-dichlorophenyl)-3,8-dihydroimidazo[4,5-d]dipyrido[2,3-b:4′,3′-f]azepine;
2-(2,6-dimethylphenyl)-3,8-dihydroimidazo[4,5-d]dipyrido[2,3-b:4′,3′-f]azepine;
2-(2-fluoro-6-methoxyphenyl)-3,8-dihydroimidazo[4,5-d]dipyrido[2,3-b:4′,3′-f]azepine;
2-(6-chloro-2-fluoro-3-methylphenyl)-3,8-dihydroimidazo[4,5-d]dipyrido[2,3-b:4′,3′-
f]azepine;
2-[2-fluoro-6-(trifluoromethyl)phenyl]-3,8-dihydroimidazo[4,5-d]dipyrido[2,3-b:4′,3′-
f]azepine;
2-(2-chloro-6-fluoro-3-methylphenyl)-3,8-dihydroimidazo[4,5-d]dipyrido[2,3-b:4′,3′-
f]azepine;
2-[3-chloro-2-fluoro-6-(trifluoromethyl)phenyl]-3,8-dihydroimidazo[4,5-d]dipyrido[2,3-
b:4′,3′-f]azepine;
2-(2-chloro-6-fluorophenyl)-3,8-dihydroimidazo[4,5-d]dipyrido[2,3-b:4′,3′-f]azepine;
2-[2-chloro-5-(trifluoromethyl)phenyl]-3,8-dihydroimidazo[4,5-d]dipyrido[2,3-b:4′,3′-
f]azepine;
2-(2,6-difluorophenyl)-3,8-dihydroimidazo[4,5-d]dipyrido[2,3-b:4′,3′-f]azepine;
2-(2,5-dichlorophenyl)-3,8-dihydroimidazo[4,5-d]dipyrido[2,3-b:4′,3′-f]azepine;
2-(2,6-dibromopyridin-4-yl)-3,8-dihydroimidazo[4,5-d]dipyrido[2,3-b:4′,3′-f]azepine;
2-(2-bromophenyl)-3,8-dihydroimidazo[4,5-d]dipyrido[2,3-b:4′,3′-f]azepine;
2-(2-methylphenyl)-3,8-dihydroimidazo[4,5-d]dipyrido[2,3-b:4′,3′-f]azepine;
2-(2-chlorophenyl)-3,8-dihydroimidazo[4,5-d]dipyrido[2,3-b:4′,3′-f]azepine;
2-(2-ethylphenyl)-3,8-dihydroimidazo[4,5-d]dipyrido[2,3-b:4′,3′-f]azepine;
2-(2,5-dimethylphenyl)-3,8-dihydroimidazo[4,5-d]dipyrido[2,3-b:4′,3′-f]azepine;
2-[2-chloro-3-(trifluoromethyl)phenyl]-3,8-dihydroimidazo[4,5-d]dipyrido[2,3-b:4′,3′-
f]azepine;
2-[2,5-bis(trifluoromethyl)phenyl]-3,8-dihydroimidazo[4,5-d]dipyrido[2,3-b:4′,3′-
f]azepine;
2-(3-chloro-2,6-difluorophenyl)-3,8-dihydroimidazo[4,5-d]dipyrido[2,3-b:4′,3′-f]azepine;
2-[2-(trifluoromethyl)phenyl]-3,8-dihydroimidazo[4,5-d]dipyrido[2,3-b:4′,3′-f]azepine;
2-(2,3-dichlorophenyl)-3,8-dihydroimidazo[4,5-d]dipyrido[2,3-b:4′,3′-f]azepine;
3-[[4-(3,8-dihydroimidazo[4,5-d]dipyrido[2,3-b:4′,3′-f]azepin-2-
ylphenyl](ethyl)amino]propanenitrile;
2-(2-chloro-3,6-difluorophenyl)-3,8-dihydroimidazo[4,5-d]dipyrido[2,3-b:4′,3′-f]azepine;
2-(2-bromopyridin-4-yl)-3,8-dihydroimidazo[4,5-d]dipyrido[2,3-b:4′,3′-f]azepine;
2-(5-bromo-2,3-dimethoxyphenyl)-3,8-dihydroimidazo[4,5-d]dipyrido[2,3-b:4′,3′-
f]azepine;
2-[3-chloro-2-fluoro-5-(trifluoromethyl)phenyl]-3,8-dihydroimidazo[4,5-d]dipyrido[2,3-
b:4′,3′-f]azepine;
2-(2-chloropyridin-4-yl)-3,8-dihydroimidazo[4,5-d]dipyrido[2,3-b:4′,3′-f]azepine;
2-(2,3-dimethylphenyl)-3,8-dihydroimidazo[4,5-d]dipyrido[2,3-b:4′,3′-f]azepine;
2-[2-fluoro-3-(trifluoromethyl)phenyl]-3,8-dihydroimidazo[4,5-d]dipyrido[2,3-b:4′,3′-
f]azepine;
2-(3-fluoro-2-methylphenyl)-3,8-dihydroimidazo[4,5-d]dipyrido[2,3-b:4′,3′-f]azepine;
2-(2-fluorophenyl)-3,8-dihydroimidazo[4,5-d]dipyrido[2,3-b:4′,3′-f]azepine;
2-(5-bromo-2-methoxyphenyl)-3,8-dihydroimidazo[4,5-d]dipyrido[2,3-b:4′,3′-f]azepine;
2-[2-fluoro-5-(trifluoromethyl)phenyl]-3,8-dihydroimidazo[4,5-d]dipyrido[2,3-b:4′,3′-
f]azepine;
2-(2-fluoro-3-methoxyphenyl)-3,8-dihydroimidazo[4,5-d]dipyrido[2,3-b:4′,3′-f]azepine;
2-(2-fluoro-5-methoxyphenyl)-3,8-dihydroimidazo[4,5-d]dipyrido[2,3-b:4′,3′-f]azepine;
2-(2,3-difluorophenyl)-3,8-dihydroimidazo[4,5-d]dipyrido[2,3-b:4′,3′-f]azepine;
2-quinolin-4-yl-3,8-dihydroimidazo[4,5-d]dipyrido[2,3-b:4′,3′-f]azepine;
1957]2-(5-fluoro-2-methoxyphenyl)-3,8-dihydroimidazo[4,5-d]dipyrido[2,3-b:4′,3′-
f]azepine;
2-(5-bromo-2-fluorophenyl)-3,8-dihydroimidazo[4,5-d]dipyrido[2,3-b:4′,3′-f]azepine;
2-(2,5-dimethoxyphenyl)-3,8-dihydroimidazo[4,5-d]dipyrido[2,3-b:4′,3′-f]azepine;
2-(2,5-difluorophenyl)-3,8-dihydroimidazo[4,5-d]dipyrido[2,3-b:4′,3′-f]azepine;
2-(3,5-dimethyl-1H-pyrrol-2-yl)-3,8-dihydroimidazo[4,5-d]dipyrido[2,3-b:4′,3′-
f]azepine;
2-(2,6-dimethoxyphenyl)-3,8-dihydroimidazo[4,5-d]dipyrido[2,3-b:4′,3′-f]azepine;
2-(4-methyl-1H-imidazol-5yl)-3,8-dihydroimidazo[4,5d]dipyrido[2,3-b:4′,3′-f]azepine;
4-(3,8-dihydroimidazo[4,5-d]dipyrido[2,3-b:4′,3′-f]azepin-2-yl)-3,5-dimethoxyphenol;
2-(2-pentafluoroethyl)-3,8-dihydroimidazo[4,5-d]dipyrido[2,3-b:4′,3′-f]azepine;
2-(5-bromo-1,3-benzodioxol-4-yl)-3,8-dihydroimidazo[4,5-d]dipyrido[2,3-b:4′,3′-
f]azepine;
4-(3,8-dihydroimidazo[4,5-d]dipyrido[2,3-b:4′,3′-f]azepin-2-yl)-4-phenylpentanenitrile;
2-[2-fluoro-4-(trifluoromethyl)phenyl]-3,8-dihydroimidazo[4,5-d]dipyrido[2,3-b:4′,3′-
f]azepine;
2-(cyclohexylmethyl)-3,8-dihydroimidazo[4,5-d]dipyrido[2,3-b:4′,3′-f]azepine;
3-bromo-2-(3,8-dihydroimidazo[4,5-d]dipyrido[2,3-b:4′,3′-f]azepin-2-yl-6-
methoxyphenol;
2-(2-fluoropyridin-4-yl)-3,8-dihydroimidazo[4,5-d]dipyrido[2,3-b:4′,3′-f]azepine;
2-biphenyl-2-yl-3,8-dihydroimidazo[4,5-d]dipyrido[2,3-b:4′,3′-f]azepine;
methyl-4-(3,8-dihydroimidazo[4,5-d]dipyrido[2,3-b:4′,3′-f]azepin-2-yl)benzoate;
2-[2-(ethylthio)phenyl]-3,8-dihydroimidazo[4,5-d]dipyrido[2,3-b:4′,3′-f]azepine;
2-(1H-pyrrol-2-yl)-3,8-dihydroimidazo[4,5-d]dipyrido[2,3-b:4′,3′-f]azepine;
2-{4-[(trifluoromethyl)thio]phenyl}-3,8-dihydroimidazo[4,5-d]dipyrido[2,3-b:4′,3′-
f]azepine;
2-(2-naphthyl)-3,8-dihydroimidazo[4,5-d]dipyrido[2,3-b:4′,3′-f]azepine;
tert-butyl[1-(3,8-dihydroimidazo[4,5-d]dipyrido[2,3-b:4′,3′-f]azepin-2-yl)-2-
phenethyl]carbamate;
2-pyrrolidin-2-yl-3,8-dihydroimidazo[4,5-d]dipyrido[2,3-b:4′,3′-f]azepine;
2-(2-chloro-6-methoxyquinolin-3-yl)-3,8-dihydroimidazo[4,5-d]dipyrido[2,3-b:4′,3′-
f]azepine;
2-(1-acetylpyrrolidin-2-yl)-3,8-dihydroimidazo[4,5-d]dipyrido[2,3-b:4′,3′-f]azepine;
2-(1,3-thiazol-2-yl)-3,8-dihydroimidazo[4,5-d]dipyrido[2,3-b:4′,3′-f]azepine;
1-acetyl-5-(3,8-dihydroimidazo[4,5-d]dipyrido[2,3-b:4′,3′-f]azepin-2-yl)pyrrolidin-3-ol;
N-[1-(3,8-dihydroimidazo[4,5-d]dipyrido[2,3-b:4′,3′-f]azepin-2-yl)ethyl]acetamide;
tert-butyl2-(3,8-dihydroimidazo[4,5-d]dipyrido[2,3-b:4′,3′-f]azepin-2-yl)-4-
hydroxypyrrolidine-1-carboxylate;
N-[4-(3,8-dihydroimidazo[4,5-d]dipyrido[2,3-b:4′,3′-f]azepin-2-yl)phenyl]acetamide;
2-[4-(difluoromethoxy)phenyl)-3,8-dihydroimidazo[4,5-d]dipyrido[2,3-b:4′,3′-f]azepine;
2-(6-chloropyridin-3-yl)-3,8-dihydroimidazo[4,5-d]dipyrido[2,3-b:4′,3′-f]azepine;
6-(3,8-dihydroimidazo[4,5-d]dipyrido[2,3-b:4′,3′-f]azepin-2-yl)-
2(methylthio)nicotinonitrile;
2-(3-fluoropyridin-4-yl)-3,8-dihydroimidazo[4,5-d]dipyrido[2,3-b:4′,3′-f]azepine;
2-(6-methoxypyridin-3-yl)-3,8-dihydroimidazo[4,5-d]dipyrido[2,3-b:4′,3′-f]azepine;
2-(6-bromopyridin-3-yl)-3,8-dihydroimidazo[4,5-d]dipyrido[2,3-b:4′,3′-f]azepine;
2-(6-bromopyridin-2-yl)-3,8-dihydroimidazo[4,5-d]dipyrido[2,3-b:4′,3′-f]azepine;
2-(1H-imidazol-4-yl)-3,8-dihydroimidazo[4,5-d]dipyrido[2,3-b:4′,3′-f]azepine;
2-(3-methoxyphenyl)-3,8-dihydroimidazo[4,5-d]dipyrido[2,3-b:4′,3′-f]azepine;
2-(4-methoxyphenyl)-3,8-dihydroimidazo[4,5-d]dipyrido[2,3-b:4′,3′-f]azepine;
2-[2-(methylthio)ethyl]-3,8-dihydroimidazo[4,5-d]dipyrido[2,3-b:4′,3′-f]azepine;
2-(piperidin-4-yl)-3,8-dihydroimidazo[4,5-d]dipyrido[2,3-b:4′,3′-f]azepine
3-[4-(3,8-dihydroimidazo[4,5-d]dipyrido[2,3-b:4′,3′-f]azepin-2-yl)piperidin-1-yl]-3-
oxopropanenitrile;
3-[3-(3,8-dihydroimidazo[4,5-d]dipyrido[2,3-b:4′,3′-f]azepin-2-yl)piperidin-1-yl]-3-
oxopropanenitrile;
3-[4-(3,8-dihydroimidazo[4,5-d]dipyrido[2,3-b:4′,3′-f]azepin-2-yl)piperidin-1-ylmethyl]-
3-oxopropanenitrile;
3-[3-(3,8-dihydroimidazo[4,5-d]dipyrido[2,3-b:4′,3′-f]azepin-2-yl)piperidin-1-ylmethyl]-
3-oxopropanenitrile;
2-[1-(trifluoroacetyl)piperidin-4-yl]3,8-dihydroimidazo[4,5-d]dipyrido[2,3-b:4′,3′-
f]azepine bis(trifluoroacetate);
2-[1-(trifluoroacetyl)piperidin-3-yl]3,8-dihydroimidazo[4,5-d]dipyrido[2,3-b:4′,3′-
f]azepine;
2-{[1-(trifluoroacetyl)piperidin-4-yl]methyl}-3,8-dihydroimidazo[4,5-d]dipyrido[2,3-
b:4′,3′-f]azepine;
2-{[1-(trifluoroacetyl)piperidin-3-yl]methyl}-3,8-dihydroimidazo[4,5-d]dipyrido[2,3-
b:4′,3′-f]azepine;
2-(1-acetylpiperidin-4-yl)-3,8-dihydroimidazo[4,5-d]dipyrido[2,3-b:4′,3′-f]azepine;
4-(3,8-dihydroimidazo[4,5-d]dipyrido[2,3-b:4′,3′-f]azepin-2-yl)piperidine-1-
carbaldehyde;
2-[(1-acetylpiperidin-4-yl)methyl]-3,8-dihydroimidazo[4,5-d]dipyrido[2,3-b:4′,3′-
f]azepine;
2-[(1-acetylpiperidin-3-yl)methyl]-3,8-dihydroimidazo[4,5-d]dipyrido[2,3-b:4′,3′-
f]azepine;
2-(1-methylpiperidin-3-yl)-3,8-dihydroimidazo[4,5-d]dipyrido[2,3-b:4′,3′-f]azepine;
4-(3,8-dihydroimidazo[4,5-d]dipyrido[2,3-b:4′,3′-f]azepin-2-yl)benzonitrile;
4-(3-hydroxy-3,8-dihydroimidazo[4,5-d]dipyrido[2,3-b:4′,3′-f]azepin-2-yl)benzonitrile;
3-(3,8-dihydroimidazo[4,5-d]dipyrido[2,3-b:4′,3′-f]azepin-2-yl)benzonitrile;
2-pyridin-3-yl-3,8-dihydroimidazo[4,5-d]dipyrido[2,3-b:4′,3′-f]azepine
2-pyridin-3-ylimidazo[4,5-d]dipyrido[2,3-b:4′,3′-f]azepin-3(8H)-ol;
2-pyridin-2-ylimidazo[4,5-d]dipyrido[2,3-b:4′,3′-f]azepin-3(8H)-ol;
2-pyridin-4-yl-3,8-dihydroimidazo[4,5-d]dipyrido[2,3-b:4′,3′-f]azepine;
2-pyridin-4-ylimidazo[4,5-d]dipyrido[2,3-b:4′,3′-f]azepin-3(8H)-ol;
2-piperidin-3-yl-3,8-dihydroimidazo[4,5-d]dipyrido[2,3-b:4′,3′-f]azepine;
2-[4-(trifluoromethyl)phenyl]-3,8-dihydroimidazo[4,5-d]dipyrido[2,3-b:4′,3′-f]azepine;
2-[6-(trifluoromethyl)pyridine-3-yl]-3,8-dihydroimidazo[4,5-d]dipyrido[2,3-b:4′,3′-
f]azepine;
2-[3-(trifluoromethyl)phenyl]-3,8-dihydroimidazo[4,5-d]dipyrido[2,3-b:4′,3′-f]azepine;
2-(3,5-dimethylisoxazol-4-yl)imidazo[4,5-d]dipyrido[2,3-b:4′,3′-f]azepin-3(8H)-ol;
2-[4-(methylthio)phenyl]-3,8-dihydroimidazo[4,5-d]dipyrido[2,3-b:4′,3′-f]azepine;
2-[4-(methylthio)phenyl]imidazo[4,5-d]dipyrido[2,3-b:4′,3′-f]azepin-3(8H)-ol;
2-[4-(methylsulfonyl)phenyl]-3,8-dihydroimidazo[4,5-d]dipyrido[2,3-b:4′,3′-f]azepine;
2-(1H-imidazol-2-yl)-3,8-dihydroimidazo[4,5-d]dipyrido[2,3-b:4′,3′-f]azepine;
2-(1-methyl-1H-imidazol-2-yl)-3,8-dihydroimidazo[4,5-d]dipyrido[2,3-b:4′,3′-f]azepine;
2-phenyl-3,8-dihydroimidazo[4,5-d]dipyrido[2,3-b:4′,3′-f]azepine;
2-phenylimidazo[4,5-d]dipyrido[2,3-b:4′,3′-f]azepin-3(8H)-ol;
2-benzyl-3,8-dihydroimidazo[4,5-d]dipyrido[2,3-b:4′,3′-f]azepine;
2-benzylimidazo[4,5-d]dipyrido[2,3-b:4′,3′-f]azepin-3(8H)-ol;
2-(2-phenethyl)-3,8-dihydroimidazo[4,5-d]dipyrido[2,3-b:4′,3′-f]azepine;
2-(2-phenethyl)imidazo[4,5-d]dipyrido[2,3-b:4′,3′-f]azepin-3(8H)-ol;
2-piperidin-4-yl-3,8-dihydroimidazo[4,5-d]dipyrido[2,3-b:4′,3′-f]azepine;
2-piperidin-4-yl imidazo[4,5-d]dipyrido[2,3-b:4′,3′-f]azepin-3(8H)-ol;
2-(piperidin-4-ylmethyl)-3,8-dihydroimidazo[4,5-d]dipyrido[2,3-b:4′,3′-f]azepine;
2-(piperidin-4-ylmethyl)imidazo[4,5-d]dipyrido[2,3-b:4′,3′-f]azepin-3(8H)-ol;
2-(piperidin-3-ylmethyl)-3,8-dihydroimidazo[4,5-d]dipyrido[2,3-b:4′,3′-f]azepine;
2-(tetrahydro-2H-pyran-4-ylmethyl)-3,8-dihydroimidazo[4,5-d]dipyrido[2,3-b:4′,3′-
f]azepine;
2-(tetrahydro-2H-pyran-4-ylmethyl)imidazo[4,5-d]dipyrido[2,3-b:4′,3′-f]azepin-3(8H)-ol;
2-(tetrahydro-2H-thiopyran-4-ylmethyl)-3,8-dihydroimidazo[4,5-d]dipyrido[2,3-b:4′,3′-
f]azepine;
2-(tetrahydro-2H-thiopyran-4-ylmethyl)imidazo[4,5-d]dipyrido[2,3-b:4′,3′-f]azepin-
3(8H)-ol;
2-[5-methyl-3-(trifluoromethyl)-1H-pyrazol-4-yl]-3,8-dihydroimidazo[4,5-
d]dipyrido[2,3-b:4′,3′-f]azepine;
2-[3-(difluoromethyl)-5-methyl-1H-pyrazol-4-yl]-3,8-dihydroimidazo[4,5-
d]dipyrido[2,3-b:4′,3′-f]azepine;
2-[1-(4-methoxybenzyl)-3-methyl-5-(trifluoromethyl)-1H-pyrazol-4-yl]-3,8-
dihydroimidazo[4,5-d]dipyrido[2,3-b:4′,3′-f]azepine;
2-[1,5-dimethyl-3-(trifluoromethyl)-1H-pyrazol-4-yl]-3,8-dihydroimidazo[4,5-
d]dipyrido[2,3-b:4′,3′-f]azepine;
049]2-[1-ethyl-5-methyl-3-(trifluoromethyl)-1H-pyrazol-4-yl]-3,8-dihydroimidazo[4,5-
d]dipyrido[2,3-b:4′,3′-f]azepine;
2-[1-(cyclopropylmethyl)-5-methyl-3-(trifluoromethyl)-1H-pyrazol-4-yl]-3,8-
dihydroimidazo[4,5-d]dipyrido[2,3-b:4′,3′-f]azepine;
2-[3-methyl-5-(pentafluoroethyl)-1H-pyrazol-4-yl]-3,8-dihydroimidazo[4,5-
d]dipyrido[2,3-b:4′,3′-f]azepine;
4-(3,8-dihydroimidazo[4,5-d]dipyrido[2,3-b:4′,3′-f]azepin-2-yl)-N,N,5-trimethyl-3-
(trifluoromethyl)-1H-pyrazole-1-sulfonamide;
2-[3-[chloro(difluoro)methyl]-5-methyl-1-(tetrahydro-2H-pyran-2-yl)-1H-pyrazol-4-yl]-
3,8-dihydroimidazo[4,5-d]dipyrido[2,3-b:4′,3′-f]azepine;
2-[3-(difluoromethyl)-5-methyl-1-(tetrahydro-2H-pyran-2-yl)-1H-pyrazol-4-yl]-3,8-
dihydroimidazo[4,5-d]dipyrido[2,3-b:4′,3′-f]azepine;
2-(3,5-dimethyl-1H-pyrazol-4-yl)-3,8-dihydroimidazo[4,5-d]dipyrido[2,3-b:4′,3′-
f]azepine;
2-(3-isobutyl-5-methyl-1H-pyrazol-4-yl)-3,8-dihydroimidazo[4,5-d]dipyrido[2,3-b:4′,3′-
f]azepine;
2-(3-ethyl-5-methyl-1H-pyrazol-4-yl)-3,8-dihydroimidazo[4,5-d]dipyrido[2,3-b:4′,3′-
f]azepine;
2-(3-butyl-5-methyl-1H-pyrazol-4-yl)-3,8-dihydroimidazo[4,5-d]dipyrido[2,3-b:4′,3′-
f]azepine;
2-(3,5-diethyl-1H-pyrazol-4-yl)-3,8-dihydroimidazo[4,5-d]dipyrido[2,3-b:4′,3′-f]azepine;
2-(5-cyclopropyl-3-methyl-1H-pyrazol-4-yl)-3,8-dihydroimidazo[4,5-d]dipyrido[2,3-
b:4′,3′-f]azepine;
2-(2-chloro-6-methylphenyl)-3,8-dihydroimidazo[4,5-d]dipyrido[2,3-b:4′,3′-f]azepine;
2-(3,8-dihydroimidazo[4,5-d]dipyrido[2,3-b:4′,3′-f]azepin-2-yl)-3-methylbenzonitrile;
2-(2,4-dimethyl-3-thienyl)-3,8-dihydroimidazo[4,5-d]dipyrido[2,3-b:4′,3′-f]azepine;
2-[4-methylsulfinyl)phenyl]-3,8-dihydroimidazo[4,5-d]dipyrido[2,3-b:4′,3′-f]azepine;
2-[4-(ethylthio)phenyl]-3,8-dihydroimidazo[4,5-d]dipyrido[2,3-b:4′,3′-f]azepine;
2-{4-[2-(5-methyl-2-furyl)propyl]phenyl}-3,8-dihydroimidazo[4,5-d]dipyrido[2,3-b:4′,3′-
f]azepine;
2-(1-benzothien-5-yl)-3,8-dihydroimidazo[4,5-d]dipyrido[2,3-b:4′,3′-f]azepine;
1-(2,4-dimethyl-1,3-thiazol-5-yl)-3,8-dihydroimidazo[4,5-d]dipyrido[2,3-b:4′,3′-
f]azepine;
2-(3-methyl-5-phenylisoxazol-4-yl)-3,8-dihydroimidazo[4,5-d]dipyrido[2,3-b:4′,3′-
f]azepine;
2-(4-chloro-1-methyl-1H-pyrazol-3-yl)-3,8-dihydroimidazo[4,5-d]dipyrido[2,3-b:4′,3′-
f]azepine;
2-(1,3,5-trimethyl-1H-pyrazol-4-yl)-3,8-dihydroimidazo[4,5-d]dipyrido[2,3-b:4′,3′-
f]azepine;
2-(5-chloro-1,3-dimethyl-1H-pyrazol-4-yl)-3,8-dihydroimidazo[4,5-d]dipyrido[2,3-
b:4′,3′-f]azepine;
2-(3,5-dimethylisoxazol-4-yl)-3,8-dihydroimidazo[4,5-d]dipyrido[2,3-b:4′,3′-f]azepine;
2-[1-(2-methoxyethyl)-2,5-dimethyl-1H-pyrrol-3-yl]-3,8-dihydroimidazo[4,5-
d]dipyrido[2,3-b:4′,3′-f]azepine;
2-(1-cyclopropyl-2,5-dimethyl-1H-pyrrol-3-yl)-3,8-dihydroimidazo[4,5-d]dipyrido[2,3-
b:4′,3′-f]azepine;
2-[2,5-dimethoxy-4-(methylthio)phenyl]-3,8-dihydroimidazo[4,5-d]dipyrido[2,3-b:4′,3′-
f]azepine;
2-(trifluoromethyl)-3,8-dihydroimidazo[4,5-d]dipyrido[2,3-b:4′,3′-f]azepine;
2-(2,4-dimethoxy-3-methylphenyl)-3,8-dihydroimidazo[4,5-d]dipyrido[2,3-b:4′,3′-
f]azepine;
2-[2-(methylthio)phenyl]-3,8-dihydroimidazo[4,5-d]dipyrido[2,3-b:4′,3′-f]azepine;
2-(2-ethoxyphenyl)-3,8-dihydroimidazo[4,5-d]dipyrido[2,3-b:4′,3′-f]azepine;
2-(2,4-dimethoxyphenyl)-3,8-dihydroimidazo[4,5-d]dipyrido[2,3-b:4′,3′-f]azepine;
2-(2,3,4,5,6-pentamethylphenyl)-3,8-dihydroimidazo[4,5-d]dipyrido[2,3-b:4′,3′-
f]azepine;
2-(2-chloro-4-methoxyphenyl)-3,8-dihydroimidazo[4,5-d]dipyrido[2,3-b:4′,3′-f]azepine;
2-(2-methyl-4-methoxyphenyl)-3,8-dihydroimidazo[4,5-d]dipyrido[2,3-b:4′,3′-f]azepine;
2-(3,8-dihydroimidazo[4,5-d]dipyrido[2,3-b:4′,3′-f]azepin-2-yl)phenol;
2-(2,5-dimethyl-4-methoxyphenyl)-3,8-dihydroimidazo[4,5-d]dipyrido[2,3-b:4′,3′-
f]azepine;
2-(2-chloro-3,4-dimethoxyphenyl)-3,8-dihydroimidazo[4,5-d]dipyrido[2,3-b:4′,3′-
f]azepine;
2-(2,3-dimethyl-4-methoxyphenyl)-3,8-dihydroimidazo[4,5-d]dipyrido[2,3-b:4′,3′-
f]azepine;
2-(2,6-dichloro-3,4-dimethoxyphenyl)-3,8-dihydroimidazo[4,5-d]dipyrido[2,3-b:4′,3′-
f]azepine;
2,4-dichloro-3-(3,8-dihydroimidazo[4,5-d]dipyrido[2,3-b:4′,3′-f]azepin-2-yl)-6-
methoxyphenol;
2-(2,4,5-trimethylphenyl)-3,8-dihydroimidazo[4,5-d]dipyrido[2,3-b:4′,3′-f]azepine;
2-(2,4-dichlorophenyl)-3,8-dihydroimidazo[4,5-d]dipyrido[2,3-b:4′,3′-f]azepine;
2-(2-chloro-4-fluorophenyl)-3,8-dihydroimidazo[4,5-d]dipyrido[2,3-b:4′,3′-f]azepine;
2-(2,4-dimethylphenyl)-3,8-dihydroimidazo[4,5-d]dipyrido[2,3-b:4′,3′-f]azepine;
2-[2-(trifluoromethoxy)phenyl]-3,8-dihydroimidazo[4,5-d]dipyrido[2,3-b:4′,3′-f]azepine;
2-[2-(difluoromethoxy)phenyl]-3,8-dihydroimidazo[4,5-d]dipyrido[2,3-b:4′,3′-f]azepine;
2-(2-methoxypyridin-3-yl)-3,8-dihydroimidazo[4,5-d]dipyrido[2,3-b:4′,3′-f]azepine;
2-[4-fluoro-2-(trifluoromethyl)phenyl]-3,8-dihydroimidazo[4,5-d]diprido[2,3-b:4′,3′-
f]azepine;
2-(2-methoxyphenyl)-3,8-dihydroimidazo[4,5-d]dipyrido[2,3-b:4′,3′-f]azepine;
2-(2,4,6-trimethylphenyl)-3,8-dihydroimidazo[4,5-d]dipyrido[2,3-b:4′,3′-f]azepine;
2-chloro-3-(3,8-dihydroimidazo[4,5-d]dipyrido[2,3-b:4′,3′-f]azepin-2-yl)-6-
methoxyphenol;
2-(3-methylpyridin-2-yl)-3,8-dihydroimidazo[4,5-d]dipyrido[2,3-b:4′,3′-f]azepine;
4-(3,8-dihydroimidazo[4,5-d]dipyrido[2,3-b:4′,3′-f]azepin-2-yl)-3-ethoxy-N,N-
diethylaniline;
2-(2,5-dimethoxy-4-bromophenyl)-3,8-dihydroimidazo[4,5-d]dipyrido[2,3-b:4′,3′-
f]azepine;
2-(2-isobutoxyphenyl)-3,8-dihydroimidazo[4,5-d]dipyrido[2,3-b:4′,3′f]azepine;
3-(3,8-dihydroimidazo[4,5-d]dipyrido[2,3-b:4′,3′-f]azepin-2-yl)pyridine-2-amine;
2-(1H-indol-4-yl)-3,8-dihydroimidazo[4,5-d]dipyrido[2,3-b:4′,3′-f]azepine;
2-{2-[(trifluormethyl)thio]phenyl}-3,8-dihydroimidazo[4,5-d]dipyrido[2,3-b:4′,3′-f]azepine;
4-bromo-3-(3,8-dihydroimidazo[4,5-d]dipyrido[2,3-b:4′,3′-f]azepin-2-yl)phenol;
2-[2-chloro-4-(methylsulfonyl)phenyl]-3,8-dihydroimidazo[4,5-d]dipyrido[2,3-b:4′,3′-
f]azepine;
2-(2,2-difluoro-1,3-benzodioxol-4-yl)-3,8-dihydroimidazo[4,5-d]dipyrido[2,3-b:4′,3′-
f]azepine;
4-(3,8-dihydroimidazo[4,5-d]dipyrido[2,3-b:4′,3′-f]azepin-2-yl)-3,5-dimethylphenol;
2-(2-chloro-4,6-dimethoxyphenyl)-3,8-dihydroimidazo[4,5-d]dipyrido[2,3-b:4′,3′-
f]azepine;
2-(3-chlorophenyl)-3,8-dihydroimidazo[4,5-d]dipyrido[2,3-b:4′,3′-f]azepine;
2-[3-fluoro-5-(trifluoromethyl)phenyl]-3,8-dihydroimidazo[4,5-d]dipyrido[2,3-b:4′,3′-
f]azepine;
2-(3,5-dichlorophenyl)-3,8-dihydroimidazo[4,5-d]dipyrido[2,3-b:4′,3′-f]azepine;
2-(3-chloro-4-methoxyphenyl)-3,8-dihydroimidazo[4,5-d]dipyrido[2,3-b:4′,3′-f]azepine;
2-(6-chloro-1,3-benzodioxol-5-yl)-3,8-dihydroimidazo[4,5-d]dipyrido[2,3-b:4′,3′-
f]azepine;
2-(4-chloro-2,6-difluorophenyl)-3,8-dihydroimidazo[4,5-d]dipyrido[2,3-b:4′,3′-f]azepine;
2-(2-chloro-5,6-difluorophenyl)-3,8-dihydroimidazo[4,5-d]dipyrido[2,3-b:4′,3′-f]azepine;
2-(2,6-dichloro-3-fluorophenyl)-3,8-dihydroimidazo[4,5-d]dipyrido[2,3-b:4′,3′-f]azepine;
2-(2-chloro-6-fluoro-3-methoxyphenyl)-3,8-dihydroimidazo[4,5-d]dipyrido[2,3-b:4′,3′-
f]azepine;
2-(2,4,6-trifluorophenyl)-3,8-dihydroimidazo[4,5-d]dipyrido[2,3-b:4′,3′-f]azepine;
2-[2-methoxy-4-(trifluoromethoxy)phenyl]-3,8-dihydroimidazo[4,5-d]dipyrido[2,3-
b:4′,3′-f]azepine;
2-(3-chloro-4-fluorophenyl)-3,8-dihydroimidazo[4,5-d]dipyrido[2,3-b:4′,3′-f]azepine;
-(4-fluoro-2-methylphenyl)-3,8-dihydroimidazo[4,5-d]dipyrido[2,3-b:4′,3′-f]azepine;
2-(2,4-dichloro-6-methoxyphenyl)-3,8-dihydroimidazo[4,5-d]dipyrido[2,3-b:4′,3′-
f]azepine;
2-(2,6-dichloro-3-methoxyphenyl)-3,8-dihydroimidazo[4,5-d]dipyrido[2,3-b:4′,3′-
f]azepine;
2-(2,6-dichloro-4-methylthiophenyl)-3,8-dihydroimidazo[4,5-d]dipyrido[2,3-b:4′,3′-
f]azepine;
2-[2,6-dichloro-4-(methylsulfinyl)phenyl]-3,8-dihydroimidazo[4,5-d]dipyrido[2,3-b:4′,3′-
f]azepine;
2-[2,6-dichloro-4-(methylsulfonyl)phenyl]-3,8-dihydroimidazo[4,5-d]dipyrido[2,3-
b:4′,3′-f]azepine;
2-(2-chloro-6-fluoro-5-methoxyphenyl)-3,8-dihydroimidazo[4,5-d]dipyrido[2,3-b:4′,3′-
f]azepine;
2-(2-chloro-6-fluoro-4-methoxyphenyl)-3,8-dihydroimidazo[4,5-d]dipyrido[2,3-b:4′,3′-
f]azepine;
[3,5-dichloro-4-(3,8-dihydroimidazo[4,5-d]dipyrido[2,3-b:4′,3′-f]azepin-2-
yl)phenyl]methanol;
[3,5-dichloro-4-(3,8-dihydroimidazo[4,5-d]dipyrido[2,3-b:4′,3′-f]azepin-2-
yl)phenyl]methanol;
3,5-dichloro-2-(3,8-dihydroimidazo[4,5-d]dipyrido[2,3-b:4′,3′-f]azepin-2-yl)phenol
5-chloro-4-(3,8-dihydroimidazo[4,5-d]dipyrido[2,3-b:4′,3′-f]azepin-2-yl)nicotinonitrile;
4-(3,8-dihydroimidazo[4,5-d]dipyrido[2,3-b:4′,3′-f]azepin-2-yl)pyridine-3,5-
dicarbonitrile;
2-(3,8-dihydroimidazo[4,5-d]dipyrido[2,3-b:4′,3′-f]azepin-2-yl)-3-fluoro-6-
methylbenzonitrile;
4-(3,8-dihydroimidazo[4,5-d]dipyrido[2,3-b:4′,3′-f]azepin-2-yl)nicotinonitrile;
2-(3,8-dihydroimidazo[4,5-d]dipyrido[2,3-b:4′,3′-f]azepin-2-yl)-3,6-difluorobenzonitrile;
3-(3,8-dihydroimidazo[4,5-d]dipyrido[2,3-b:4′,3′-f]azepin-2-yl)-2-fluoro-4-
(trifluoromethyl)benzonitrile;
3-(3,8-dihydroimidazo[4,5-d]dipyrido[2,3-b:4′,3′-f]azepin-2-yl)-4-methoxybenzonitrile;
2-(3,8-dihydroimidazo[4,5-d]dipyrido[2,3-b:4′,3′-f]azepin-2-yl)-5,6-
dimethoxybenzonitrile;
2-(3,8-dihydroimidazo[4,5-d]dipyrido[2,3-b:4′,3′-f]azepin-2-yl)-4,5-
dimethoxyisophthalonitrile;
2-(3,8-dihydroimidazo[4,5-d]dipyrido[2,3-b:4′,3′-f]azepin-2-yl)-4-hydroxy-5-
methoxyisophthalonitrile;
4-(3,8-dihydroimidazo[4,5-d]dipyrido[2,3-b:4′,3′-f]azepin-2-yl)-2,5-
dimethoxybenzonitrile;
2-(3,8-dihydroimidazo[4,5-d]dipyrido[2,3-b:4′,3′-f]azepin-2-yl)-5-
(methylsulfonyl)benzonitrile;
2-(3,8-dihydroimidazo[4,5-d]dipyrido[2,3-b:4′,3′-f]azepin-2-yl)-3,5-
dimethoxybenzonitrile;
2-(3,8-dihydroimidazo[4,5-d]dipyrido[2,3-b:4′,3′-f]azepin-2-yl)-3,5-difluorobenzonitrile;
2-(3,8-dihydroimidazo[4,5-d]dipyrido[2,3-b:4′,3′-f]azepin-2-yl)-3,4-difluorobenzonitrile;
2-(3,8-dihydroimidazo[4,5-d]dipyrido[2,3-b:4′,3′-f]azepin-2-yl)-3-fluoro-6-
methoxybenzonitrile;
3-(3,8-dihydroimidazo[4,5-d]dipyrido[2,3-b:4′,3′-f]azepin-2-yl)-6-fluorobenzonitrile;
2-(3,8-dihydroimidazo[4,5-d]dipyrido[2,3-b:4′,3′-f]azepin-2-yl)-3-fluoro-4-
methoxybenzonitrile;
2-(3,8-dihydroimidazo[4,5-d]dipyrido[2,3-b:4′,3′-f]azepin-2-yl)-3-fluoro-5-
methoxybenzonitrile;
2-(3,8-dihydroimidazo[4,5-d]dipyrido[2,3-b:4′,3′-f]azepin-2-yl)-5-
(methylthio)isophthalonitrile;
2-(3,8-dihydroimidazo[4,5-d]dipyrido[2,3-b:4′,3′-f]azepin-2-yl)-5-
(hydroxymethyl)isophthalonitrile;
2-(3,8-dihydroimidazo[4,5-d]dipyrido[2,3-b:4′,3′-f]azepin-2-yl)-5-
(hydroxymethyl)benzonitrile;
[3,5-dichloro-4-(3,8-dihydroimidazo[4,5-d]dipyrido[2,3-b:4′,3′-f]azepin-2-
yl)phenyl]acetonitrile;
5-(cyanomethyl)-2-(3,8-dihydroimidazo[4,5-d]dipyrido[2,3-b:4′,3′-f]azepin-2-
yl)isophthalonitrile;
5-(cyanomethyl)-2-(3,8-dihydroimidazo[4,5-d]dipyrido[2,3-b:4′,3′-f]azepin-2-
yl)benzonitrile;
2-(3,8-dihydroimidazo[4,5-d]dipyrido[2,3-b:4′,3′-f]azepin-2-yl)-5-
(methylsulfinyl)benzonitrile;
2-(3,8-dihydroimidazo[4,5-d]dipyrido[2,3-b:4′,3′-f]azepin-2-yl)5-
(methylsulfonyl)isophthalonitrile;
2-(3,8-dihydroimidazo[4,5-d]dipyrido[2,3-b:4′,3′-f]azepin-2-yl)-5-hydroxybenzonitrile;
1-[3,5-dichloro-4-(3,8-dihydroimidazo[4,5-d]dipyrido[2,3-b:4′,3′-f]azepin-2-yl)-2-
phenyl]-N,N-dimethylmethanamine;
2-[2,6-dichloro-4-(morpholin-4-ylmethyl)phenyl]-3,8-dihydroimidazo[4,5-
d]dipyrido[2,3-b:4′,3′-f]azepine;
-[2,6-dichloro-4-(thiomorpholin-4-ylmethyl)phenyl]-3,8-dihydroimidazo[4,5-
d]dipyrido[2,3-b:4′,3′-f]azepine;
2-[2,6-dichloro-4-(methoxymethyl)phenyl]-3,8-dihydroimidazo[4,5-d]dipyrido[2,3-
b:4′,3′-f]azepine;
2-[2,6-dichloro-4-(ethylthio)phenyl]-3,8-dihydroimidazo[4,5-d]dipyrido[2,3-b:4′,3′-
f]azepine;
2-[2,6-dichloro-4-(isopropylthio)phenyl]-3,8-dihydroimidazo[4,5-d]dipyrido[2,3-b:4′,3′-
f]azepine;
2-[2,6-dichloro-4-(ethylsulfinyl)phenyl]-3,8-dihydroimidazo[4,5-d]dipyrido[2,3-b:4′,3′-
f]azepine;
2-[2,6-dichloro-4-(ethylsulfonyl)phenyl]-3,8-dihydroimidazo[4,5-d]dipyrido[2,3-b:4′,3′-
f]azepine;
2-[2,6-dichloro-4-(isopropylsulfinyl)phenyl]-3,8-dihydroimidazo[4,5-d]dipyrido[2,3-
b:4′,3′-f]azepine;
2-[2,6-dichloro-4-(isopropylsulfonyl)phenyl]-3,8-dihydroimidazo[4,5-d]dipyrido[2,3-
b:4′,3′-f]azepine;
2-(3,8-dihydroimidazo[4,5-d]dipyrido[2,3-b:4′,3′-f]azepin-2-yl)-5-
(isopropylsulfonyl)benzonitrile;
2-(3,8-dihydroimidazo[4,5-d]dipyrido[2,3-b:4′,3′-f]azepin-2-yl)-5-
(ethylthio)isophthalonitrile;
3-chloro-2-(3,8-dihydroimidazo[4,5-d]dipyrido[2,3-b:4′,3′-f]azepin-2-yl)-5-
(ethylthio)benzonitrile;
2-(3,8-dihydroimidazo[4,5-d]dipyrido[2,3-b:4′,3′-f]azepin-2-yl)-5-
(isopropylthio)isophthalonitrile;
3-chloro-2-(3,8-dihydroimidazo[4,5-d]dipyrido[2,3-b:4′,3′-f]azepin-2-yl)-5-
(isopropylthio)benzonitrile;
2-(3,8-dihydroimidazo[4,5-d]dipyrido[2,3-b:4′,3′-f]azepin-2-yl)-5-
(isopropylsulfinyl)isophthalonitrile;
3-chloro-2-(3,8-dihydroimidazo[4,5-d]dipyrido[2,3-b:4′,3′-f]azepin-2-yl)-5-
(isopropylsulfinyl)benzo-nitrile;
2-[3,5-dichloro-4-(3,8-dihydroimidazo[4,5-d]dipyrido[2,3-b:4′,3′-f]azepin-2-
yl)phenoxy]acetonitrile;
2-[3,5-dichloro-4-(3,8-dihydroimidazo[4,5-d]dipyrido[2,3-b:4′,3′-f]azepin-2-
yl)phenoxy]propanenitrile;
5-(1-cyanoethoxy)-2-(3,8-dihydroimidazo[4,5-d]dipyrido[2,3-b:4′,3′-f]azepin-2-
yl)isophthalonitrile;
5-(1-cyanoethoxy)-2-(3,8-dihydroimidazo[4,5-d]dipyrido[2,3-b:4′,3′-f]azepin-2-
yl)benzonitrile;
5-(cyanomethoxy)-2-(3,8-dihydroimidazo[4,5-d]dipyrido[2,3-b:4′,3′-f]azepin-2-
yl)isophthalonitrile;
2-[3,5-dichloro-4-(3,8-dihydroimidazo[4,5-d]dipyrido[2,3-b:4′,3′-f]azepin-2-
yl)phenoxy]propanamide;
2-[3,5-dicyano-4-(3,8-dihydroimidazo[4,5-d]dipyrido[2,3-b:4′,3′-f]azepin-2-
yl)phenoxy]propanamide;
5-(2-amino-1-methyl-2-oxoethoxy)-3-cyano-2-(3,8-dihydroimidazo[4,5-d]dipyrido[2,3-
b:4′,3′-f]azepin-2-yl)benzamide;
5-(2-amino-1-methyl-2-oxoethoxy)-2-(3,8-dihydroimidazo[4,5-d]dipyrido[2,3-b:4′,3′-
f]azepin-2-yl)isophthalamide;
2-[3,5-dichloro-4-(3,8-dihydroimidazo[4,5-d]dipyrido[2,3-b:4′,3′-f]azepin-2-
yl)phenoxy]acetamide;
2-[3,5-dicyano-4-(3,8-dihydroimidazo[4,5-d]dipyrido[2,3-b:4′,3′-f]azepin-2-
yl)phenoxy]acetamide;
2-cyano-2-[3,5-dichloro-4-(3,8-dihydroimidazo[4,5-d]dipyrido[2,3-b:4′,3′-f]azepin-2-
yl)phenoxy]-N,N-dimethylacetamide;
2-(3,8-dihydroimidazo[4,5-d]dipyrido[2,3-b:4′,3′-f]azepin-2-yl)isophthalonitrile;
2-(3,8-dihydroimidazo[4,5-d]dipyrido[2,3-b:4′,3′-f]azepin-2-yl)benzonitrile;
2-(3,8-dihydroimidazo[4,5-d]dipyrido[2,3-b:4′,3′-f]azepin-2-yl)-4-hydroxybenzonitrile;
4-(3,8-dihydroimidazo[4,5-d]dipyrido[2,3-b:4′,3′-f]azepin-2-yl)-1,3-benzodioxole-5-
carbonitrile;
3-chloro-2-(3,8-dihydroimidazo[4,5-d]dipyrido[2,3-b:4′,3′-f]azepin-2-yl)benzonitrile;
3-fluoro-4-methyl-2-(3,8-dihydroimidazo[4,5-d]dipyrido[2,3-b:4′,3′-f]azepin-2-
yl)benzonitrile;
3-fluoro-2-(3,8-dihydroimidazo[4,5-d]dipyrido[2,3-b:4′,3′-f]azepin-2-yl)benzonitrile;
3-methoxy-2-(3,8-dihydroimidazo[4,5-d]dipyrido[2,3-b:4′,3′-f]azepin-2-yl)benzonitrile;
5-fluoro-2-(3,8-dihydroimidazo[4,5-d]dipyrido[2,3-b:4′,3′-f]azepin-2-yl)benzonitrile;
3-chloro-6-methoxy-2-(3,8-dihydroimidazo[4,5-d]dipyrido[2,3-b:4′,3′-f]azepin-2-
yl)benzonitrile;
2-(3,8-dihydroimidazo[4,5-d]dipyrido[2,3-b:4′,3′-f]azepin-2-yl)-4-
methoxyisophthalonitrile;
tert-butyl[4-(3,8-dihydroimidazo[4,5-d]dipyrido[2,3-b:4′,3′-f]azepin-2-yl)pyridin-3-
yl]carbamate;
tert-butyl[4-(3-hydroxy-3,8-dihydroimidazo[4,5-d]dipyrido[2,3-b:4′,3′-f]azepin-2-
yl)pyridin-3-yl]carbamate;
2-(3,5-dimethylpyridin-4-yl)-3,8-dihydroimidazo[4,5-d]dipyrido[2,3-b:4′,3′-f]azepine;
3,5-dichloro-4-(3,8-dihydroimidazo[4,5-d]dipyrido[2,3-b:4′,3′-f]azepin-2-yl)phenol;
2-(2,6-dichloro-4-methoxyphenyl)-3,8-dihydroimidazo[4,5-d]dipyrido[2,3-b:4′,3′-
f]azepine;
2-(2,6-dichloro-4-ethoxyphenyl)-3,8-dihydroimidazo[4,5-d]dipyrido[2,3-b:4′,3′-
f]azepine;
2-(2,6-dichloro-4-isopropoxyphenyl)-3,8-dihydroimidazo[4,5-d]dipyrido[2,3-b:4′,3′-
f]azepine;
2-[2,6-dichloro-4-(trifluoromethoxy)phenyl]-3,8-dihydroimidazo[4,5-d]dipyrido[2,3-
b:4′,3′-f]azepine;
2-(3-methylpyridin-4-yl)-3,8-dihydroimidazo[4,5-d]dipyrido[2,3-b:4′,3′-f]azepine;
2-(2-methylpyridin-3-yl)-3,8-dihydroimidazo[4,5-d]dipyrido[2,3-b:4′,3′-f]azepine;
(2,4-dimethylpyridin-3-yl)-3,8-dihydroimidazo[4,5-d]dipyrido[2,3-b:4′,3′-f]azepine;
2-(5-chloro-2-ethoxyphenyl)-3,8-dihydroimidazo[4,5-d]dipyrido[2,3-b:4′,3′-f]azepine;
2-(5-chloro-2-methoxyphenyl)-3,8-dihydroimidazo[4,5-d]dipyrido[2,3-b:4′,3′-f]azepine;
2-(5-bromo-2-ethoxyphenyl)-3,8-dihydroimidazo[4,5-d]dipyrido[2,3-b:4′,3′-f]azepine;
2-(2,3-difluoro-6-methoxyphenyl)-3,8-dihydroimidazo[4,5-d]dipyrido[2,3-b:4′,3′-
f]azepine;
2-[2,6-dichloro-4-(trifluoromethyl)phenyl]-3,8-dihydroimidazo[4,5-d]dipyrido[2,3-
b:4′,3′-f]azepine;
3,5-dichloro-4-(3,8-dihydroimidazo[4,5-d]dipyrido[2,3-b:4′,3′-f]azepin-2-yl)-N,N-
dimethylaniline;
2-(2,6-dichloro-4-fluorophenyl)-3,8-dihydroimidazo[4,5-d]dipyrido[2,3-b:4′,3′-f]azepine;
3-chloro-2-(3,8-dihydroimidazo[4,5-d]dipyrido[2,3-b:4′,3′-f]azepin-2-yl)-5-
methoxybenzonitrile;
2-(3,8-dihydroimidazo[4,5-d]dipyrido[2,3-b:4′,3′-f]azepin-2-yl)-5-
methoxyisophthalonitrile;
3-chloro-2-(3,8-dihydroimidazo[4,5-d]dipyrido[2,3-b:4′,3′-f]azepin-2-yl)-5-
ethoxybenzonitrile;
2-(3,8-dihydroimidazo[4,5-d]dipyrido[2,3-b:4′,3′-f]azepin-2-yl)-5-
ethoxyisophthalonitrile;
3-chloro-2-(3,8-dihydroimidazo[4,5-d]dipyrido[2,3-b:4′,3′-f]azepin-2-yl)-5-
isopropoxybenzonitrile;
2-(3,8-dihydroimidazo[4,5-d]dipyrido[2,3-b:4′,3′-f]azepin-2-yl)-5-
isopropoxyisophthalonitrile;
2-(3,8-dihydroimidazo[4,5-d]dipyrido[2,3-b:4′,3′-f]azepin-2-yl)-5-
(trifluoromethoxy)isophthalonitrile;
3-chloro-2-(3,8-dihydroimidazo[4,5-d]dipyrido[2,3-b:4′,3′-f]azepin-2-yl)-5-
(trifluoromethyl)benzonitrile;
3-chloro-2-(3,8-dihydroimidazo[4,5-d]dipyrido[2,3-b:4′,3′-f]azepin-2-yl)-5-
(dimethylamino)benzonitrile;
3-chloro-2-(3,8-dihydroimidazo[4,5-d]dipyrido[2,3-b:4′,3′-f]azepin-2-yl)-5-
fluorobenzonitrile
2-tert-butyl-10-fluoro-3,8-dihydroimidazo[4,5-d]pyrido[2,3-b][1]benzazepine;
2-cyclopropyl-10-fluoro-3,8-dihydroimidazo[4,5-d]pyrido[2,3-b][1]benzazepine;
2-cyclohexyl-10-fluoro-3,8-dihydroimidazo[4,5-d]pyrido[2,3-b][1]benzazepine;
10-fluoro-2-isobutyl-3,8-dihydroimidazo[4,5-d]pyrido[2,3-b][1]benzazepine;
2-cyclopentyl-10-fluoro-3,8-dihydroimidazo[4,5-d]pyrido[2,3-b][1]benzazepine;
10-fluoro-2-(tetrahydrofuran-3-yl)-3,8-dihydroimidazo[4,5-d]pyrido[2,3-
b][1]benzazepine;
2-cyclohex-3-en-1-yl-10-fluoro-3,8-dihydroimidazo[4,5-d]pyrido[2,3-b][1]benzazepine;
Trans ethyl(1R,S)-2-(10-fluoro-3,8-dihydroimidazo[4,5-d]pyrido[2,3-b][1]benzazepin-2-
yl)cyclopropanecarboxylate;
2-bicyclo[2.2.1]hept-5-en-2-yl-10-fluoro-3,8-dihydroimidazo[4,5-d]pyrido[2,3-
b][1]benzazepine;
[(1S)-2-(10-fluoro-3,8-dihydroimidazo[4,5-d]pyrido[2,3-b][1]benzazepin-2-
yl)cyclopropyl]methanol trifluoroacetate;
2-(1-ethylpentyl)-10-fluoro-3,8-dihydroimidazo[4,5-d]pyrido[2,3-b][1]benzazepine;
4-ethyl-4-(10-fluoro-3,8-dihydroimidazo[4,5-d]pyrido[2,3-b][1]benzazepin-2-
yl)hexanenitrile;
2-cyclopentyl-10,11-difluoro-3,8-dihydroimidazo[4,5-d]pyrido[2,3-b][1]benzazepine;
2-(1-ethylpentyl)-10,11-difluoro-3,8-dihydroimidazo[4,5-d]pyrido[2,3-b][1]benzazepine;
3-[1-(10-fluoro-3,8-dihydroimidazo[4,5-d]pyrido[2,3-b][1]benzazepin-2-
yl)cyclohexyl]propanenitrile;
4-(10-fluoro-3,8-dihydroimidazo[4,5-d]pyrido[2,3-b][1]benzazepin-2-yl)-4-
methylpentanenitrile;
2-(1-ethylpropyl)-10-fluoro-3,8-dihydroimidazo[4,5-d]pyrido[2,3-b][1]benzazepine;
10-fluoro-2-[2-(methylthio)ethyl]-3,8-dihydroimidazo[4,5-d]pyrido[2,3-
b][1]benzazepine;
10-fluoro-2-[2-(methylsulfinyl)ethyl]-3,8-dihydroimidazo[4,5-d]pyrido[2,3-
b][1]benzazepine;
2-[(benzyloxy)methyl]-10-fluoro-3,8-dihydroimidazo[4,5-d]pyrido[2,3-
b][1]benzazepine;
Cis-[4-(10-fluoro-3,8-dihydroimidazo[4,5-d]pyrido[2,3-b][1]benzazepin-2-
yl)cyclohexyl]methanol;
Trans-[4-(10-fluoro-3,8-dihydroimidazo[4,5-d]pyrido[2,3-b][1]benzazepin-2-
yl)cyclohexyl]methanol;
Cis-4-(10-fluoro-3,8-dihydroimidazo[4,5-d]pyrido[2,3-b][1]benzazepin-2-
yl)cyclohexanol;
Trans-4-(10-fluoro-3,8-dihydroimidazo[4,5-d]pyrido[2,3-b][1]benzazepin-2-
yl)cyclohexanol;
Trans-[4-(10-fluoro-3,8-dihydroimidazo[4,5-d]pyrido[2,3-b][1]benzazepin-2-
yl)cyclohexyl]acetonitrile;
2-[4-(10-fluoro-3,8-dihydroimidazo[4,5-d]pyrido[2,3-b][1]benzazepin-2-
yl)cyclohexyl]acetamide;
tert-butyl4-(10-fluoro-3,8-dihydroimidazo[4,5-d]pyrido[2,3-b][1]benzazepin-2-
yl)piperidine-1-carboxylate;
10-fluoro-2-piperidin-4-yl-3,8-dihydroimidazo[4,5-d]pyrido[2,3-b][1]benzazepine;
3-[4-(10-fluoro-3,8-dihydroimidazo[4,5-d]pyrido[2,3-b][1]benzazepin-2-yl)piperidin-1-
yl]-3-oxopropanenitrile;
(1-acetylpiperidin-4-yl)-10-fluoro-3,8-dihydroimidazo[4,5-d]pyrido[2,3-
b][1]benzazepine;
10-fluoro-2-[1-(methylsulfonyl)piperidin-4-yl]-3,8-dihydroimidazo[4,5-d]pyrido[2,3-
b][1]benzazepine;
[4-(10-fluoro-3,8-dihydroimidazo[4,5-d]pyrido[2,3-b][1]benzazepin-2-yl)piperidin-1-
yl]acetonitrile;
ethyl[4-(10-fluoro-3,8-dihydroimidazo[4,5-d]pyrido[2,3-b][1]benzazepin-2-yl)piperidin-
1-yl]acetate;
2-[4-(10-fluoro-3,8-dihydroimidazo[4,5-d]pyrido[2,3-b][1]benzazepin-2-yl)piperidin-1-
yl]ethanol;
3-[4-(10-fluoro-3,8-dihydroimidazo[4,5-d]pyrido[2,3-b][1]benzazepin-2-yl)piperidin-1-
yl]propan-1-ol;
4-[4-(10-fluoro-3,8-dihydroimidazo[4,5-d]pyrido[2,3-b][1]benzazepin-2-yl)piperidin-1-
yl]butanenitrile;
10-fluoro-2-[3-methyl-5-(trifluoromethyl)-1H-pyrazol-4-yl]-3,8-dihydroimidazo[4,5-
d]pyrido[2,3-b][1]benzazepine;
2-[3-(difluoromethyl)-5-methyl-1H-pyrazol-4-yl]-10-fluoro-3,8-dihydroimidazo[4,5-
d]pyrido[2,3-b][1]benzazepine;
2-(3,5-dimethyl-1H-pyrazol-4-yl)-10-fluoro-3,8-dihydroimidazo[4,5-d]pyrido[2,3-
b][1]benzazepine;
2-(3,5-diethyl-1H-pyrazol-4-yl)-10-fluoro-3,8-dihydroimidazo[4,5-d]pyrido[2,3-
b][1]benzazepine;
[4-(10-fluoro-3,8-dihydroimidazo[4,5-d]pyrido[2,3-b][1]benzazepin-2-yl)-5-methyl-3-
(trifluoromethyl)-1H-pyrazol-1-yl]acetonitrile;
2-(2-chloro-6-methylphenyl)-10-fluoro-3,8-dihydroimidazo[4,5-d]pyrido[2,3-
b][1]benzazepine trifluoroacetate;
2-(10-fluoro-1,8-dihydroimidazo[4,5-d]pyrido[2,3-b][1]benzazepin-2-yl)-3-
methylbenzonitrile trifluoroacetate;
2-(2,6-dimethylphenyl)-10-fluoro-3,8-dihydroimidazo[4,5-d]pyrido[2,3-
b][1]benzazepine;
2-(3,5-dichloropyridin-4-yl)-10-fluoro-3,8-dihydroimidazo[4,5-d]pyrido[2,3-
b][1]benzazepine;
2-(2,4-dimethylpyridin-3-yl)-10-fluoro-3,8-dihydroimidazo[4,5-d]pyrido[2,3-
b][1]benzazepine;
2-(3,5-dichloropyridin-4-yl)-3,8-dihydroimidazo[4,5-d]dipyrido[2,3-b:3′,4′-f]azepine;
2-(2,6-dichlorophenyl)-3,8-dihydroimidazo[4,5-d]dipyrido[2,3-b:3′,4′-f]azepine;
2-(2,6-difluorophenyl)-3,8-dihydroimidazo[4,5-d]dipyrido[2,3-b:3′,4′-f]azepine;
2-(2-chloro-6-fluorophenyl)-3,8-dihydroimidazo[4,5-d]dipyrido[2,3-b:3′,4′-f]azepine;
2-(2,6-dimethylphenyl)-3,8-dihydroimidazo[4,5-d]dipyrido[2,3-b:3′,4′-f]azepine;
4-(3,8-dihydroimidazo[4,5-d]dipyrido[2,3-b:3′,4′-f]azepin-2-yl)-4-ethylhexanenitrile;
2-[1-(4-methoxybenzyl)-5-methyl-3-(trifluoromethyl)-1H-pyrazol-4-yl]imidazo[4,5-
d]dipyrido[2,3-b:3′,4′-f]azepin-3(8H)-ol;
2-[1-(4-methoxybenzyl)-5-methyl-3-(trifluoromethyl)-1H-pyrazol-4-yl]-3,8-
dihydroimidazo[4,5-d]dipyrido[2,3-b:3′,4′-f]azepine;
2-[5-methyl-3-(trifluoromethyl)-1H-pyrazol-4-yl]imidazo[4,5-d]dipyrido[2,3-b:3′,4′-
f]azepin-3(8H)-ol;
2-[5-methyl-3-(trifluoromethyl)-1H-pyrazol-4-yl]-3,8-dihydroimidazo[4,5-
d]dipyrido[2,3-b:3′,4′-f]azepine;
2-(2,3-dimethylphenyl)imidazo[4,5-d]dipyrido[2,3-b:3′,4′-f]azepin-3(8H)-ol;
2-(2,3-dimethylphenyl)-1,8-dihydroimidazo[4,5-d]dipyrido[2,3-b:3′,4′-f]azepine;
2-[2-(dimethylamino)-pyridin-3-yl]imidazo[4,5-d]dipyrido[2,3-b:3′,4′-f]azepin-3(8H)-ol;
3-(3,8-dihydroimidazo[4,5-d]dipyrido[2,3-b:3′,4′-f]azepin-2-yl)-N,N-dimethylpyridin-2-
amine;
2-(2-cyano-6-fluorophenyl)-3,8-dihydroimidazo[4,5-d]dipyrido[2,3-b:3′,4′-f]azepine
N-[4-methyl-3-(9H-pyrimido[4,5-b]indol-7-yl)phenyl]-3-(trifluoromethypbenzamide;
3-(4-Methyl-1H-imidazol-1-yl)-N-[4-methyl-3-(9H-pyrimido[4,5-b]indol-7-yl)phenyl]-5-
(trifluoromethyl)benzamide;
3-fluoro-N-[4-methyl-3-(9H-pyrimido[4,5-b]indol-7-yl)phenyl]-5-
(trifluoromethyl)benzamide;
3-(4-formyl-1H-imidazol-1-yl)-N-[4-methyl-3-(9H-pyrimido[4,5-b]indol-7-yl)phenyl]-5-
(trifluoromethyl)benzamide;
3-[4-(hydroxymethyl)-1H-imidazol-1-yl]-N-[4-methyl-3-(9H-pyrimido[4,5-b]indol-7-
yl)phenyl]-5-(trifluoromethyl)benzamide;
3-4-[(methylamino)methyl]-1H-imidazol-1-yl-N-[4-methyl-3-(9H-pyrimido[4,5-b]-indol-
7-yl)phenyl]-5-(trifluoromethyl)benzamide;
3-(4-methylpiperazin-1-yl)-N-[4-methyl-3-(9H-pyrimido[4,5-b]indol-7-yl)phenyl]-5-
(trifluoromethyl)benzamide;
N-[4-methyl-3-(9H-pyrimido[4,5-b]indol-6-yl)phenyl]-3-(trifluoromethyl)benzamide;
4-fluoro-N-[4-methyl-3-(9H-pyrimido[4,5-b]indol-6-yl)phenyl]-3-
(trifluoromethyl)benzamide;
N-[4-methyl-3-(9H-pyrimido[4,5-b]indol-7-yl)phenyl]-N-p-(trifluoromethyl)phenyl]urea;
N-[4-methyl-3-(9H-pyrimido[4,5-b]indol-6-yl)phenyl]-N′-[3-
(trifluoromethyl)phenyl]urea;
4-methyl-3-(9H-pyrimido[4,5-b]indol-6-yl)-N-[3-(trifluoromethyl)phenyl]benzamide;
4-chloro-3-(9H-pyrimido[4,5-b]indol-7-yl)-N-[3-(trifluoromethyl)phenyl]benzamide;
4-cyano-3-(9H-pyrimido[4,5-b]indol-7-yl)-N-[3-(trifluoromethyl)phenyl]benzamide;
2-fluoro-N-[4-methyl-3-(9H-pyrimido[4,5-b]indol-7-yl)phenyl]-3-
(trifluoromethyl)benzamide;
4-fluoro-N-[4-methyl-3-(9H-pyrimido[4,5-b]indol-7-yl)phenyl]-3-
(trifluoromethyl)benzamide;
2-fluoro-N-[4-methyl-3-(9H-pyrimido[4,5-b]indol-7-yl)phenyl]-5-
(trifluoromethyl)benzamide;
3-fluoro-N-[4-methyl-3-(9H-pyrimido[4,5-b]indol-6-yl)phenyl]benzamide;
N-[4-methyl-3-(9H-pyrimido[4,5-b]indol-7-yl)phenyl]-2,5-
bis(trifluoromethyl)benzamide;
3-chloro-2-fluoro-N-[4-methyl-3-(9H-pyrimido[4,5-b]indol-7-yl)phenyl]-5-
(trifluoromethyl)benzamide;
N-[4-methyl-3-(9H-pyrimido[4,5-b]indol-7-yl)phenyl]-3,5-
bis(trifluoromethyl)benzamide;
4-methoxy-N-[4-methyl-3-(9H-pyrimido[4,5-b]indol-7-yl)phenyl]-3-
(trifluoromethyl)benzamide;
3-methoxy-N-[4-methyl-3-(9H-pyrimido[4,5-b]indol-7-yl)phenyl]benzamide;
N-[4-methyl-3-(9H-pyrimido[4,5-b]indol-7-yl)phenyl]-2-(trifluoromethyl)benzamide;
N-[4-fluoro-3-(9H-pyrimido[4,5-b]indol-7-yl)phenyl]-3-(trifluoromethyl)benzamide;
N-[3-(9H-pyrimido[4,5-b]-indol-7-yl)phenyl]-3-(trifluoromethyl)benzamide;
3-chloro-N-[4-methyl-3-(9H-pyrimido[4,5-b]indol-7-yl)phenyl]benzamide;
N-[4-methyl-3-(9H-pyrimido[4,5-b]indol-7-yl)phenyl]-3-(pentafluoro[80 (6)-
sulfanyl)benzamide;
N-[4-chloro-3-(9H-pyrimido[4,5-b]indol-7-yl)phenyl]-3-(trifluoromethyl)benzamide;
N-[4-methyl-3-(9H-pyrimido[4,5-b]indol-7-yl)phenyl]-4-(trifluoromethyl)pyridine-2-
carboxamide;
3-(1H-imidazol-1-yl)-N-(4-methyl-3-(9H-pyrimido[4,5-b]indol-7-yl)phenyl]-5-
(trifluoromethyl)benzamide;
N-[4-methyl-3-(9H-pyrimido[4,5-b]indol-7-yl)phenyl]-3-(1H-1,2,4-triazol-1-yl)-5-
(trifluoromethyl)benzamide;
3-{4-[(dimethylamino)methyl]-1H-imidazol-1-yl}-N-[4-methyl-3-(9H-pyrimido[4,5-
b]indol-7-yl)phenyl]-5-(trifluoromethyl)benzamide;
N-[4-methyl-3-(9H-pyrimido[4,5-b]indol-7-yl)phenyl]-3-morpholin-4-yl-5-
(trifluoromethyl)benzamide;
N-[4-methyl-3-(9H-pyrimido[4,5-b]indol-7-yl)phenyl]-3-piperazin-1-yl-5-
(trifluoromethyl)benzamide;
3-(4-hydroxypiperidin-1-yl)-N-[4-methyl-3-(9H-pyrimido[4,5-b]indol-7-yl)phenyl]-5-
(trifluoromethyl)benzamide;
3-(3-hydroxypiperidin-1-yl)-N-[4-methyl-3-(9H-pyrimido[4,5-b]indol-7-yl)phenyl]-5-
(trifluoromethyl)benzamide;
N-[4-methyl-3-(9H-pyrimido[4,5-b]indol-7-yl)phenyl]-3-[(2-morpholin-4-
ylethyl)amino]-5-(trifluoromethyl)benzamide;
3-[4-(2-hydroxyethyl)piperazin-1-yl]-N-[4-methyl-3-(9H-pyrimido[4,5-b]indol-7-
yl)phenyl]-5-(trifluoromethyl)benzamide;
3-{[3-(dimethylamino)propyl]amino)-N-[4-methyl-3-(9H-pyrimido[4,5-b]indol-7-
yl)phenyl]-5-(trifluoromethyl)benzamide;
3-(3-hydroxypyrrolidin-1-yl)-N-[4-methyl-3-(9H-pyrimido[4,5-b]indol-7-yl)phenyl]-5-
(trifluoromethyl)benzamide;
3-{[3-(1H-imidazol-1-yl)-propyl]amino}-N-[4-methyl-3-(9H-pyrimido[4,5-b]indol-7-
yl)phenyl]-5-(trifluoromethyl)benzamide;
3-(dimethylamino)-N-[4-methyl-3-(9H-pyrimido[4,5-b]indol-7-yl)phenyl]-5-
(trifluoromethyl)benzamide;
3-[3-(dimethylamino)-pyrrolidin-1-yl]-N-[4-methyl-3-(9H-pyrimido[4,5-b]indol-7-
yl)phenyl]-5-(trifluoromethyl)benzamide;
3-{[2-(dimethylamino)ethyl]amino}-N-[4-methyl-3-(9H-pyrimido[4,5-b]indol-7-
yl)phenyl]-5-(trifluoromethyl)benzamide;
4-fluoro-N-[4-methyl-3-(9H-pyrimido[4,5-b]indol-6-yl)phenyl]-3-
(trifluoromethyl)benzamide;
3-fluoro-N-[4-methyl-3-(9H-pyrimido[4,5-b]indol-6-yl)phenyl]-5-
(trifluoromethyl)benzamide;
3-fluoro-N-[4-methyl-3-(9H-pyrimido[4,5-b]indol-6-yl)phenyl]benzamide;
N-[4-methyl-3-(9H-pyrimido[4,5-b]indol-6-yl)phenyl]-2,5-
bis(trifluoromethyl)benzamide;
3-chloro-2-fluoro-N-[4-methyl-3-(9H-pyrimido[4,5-b]indol-6-yl)phenyl]-5-
(trifluoromethyl)benzamide;
4-methyl-3-(9H-pyrimido-[4,5-b]indol-7-yl)-N-[3-(trifluoromethyl)phenyl]benzamide;
4-methyl-N-(3-methyl-phenyl)-3-(9H-pyrimido[4,5-b]indol-7-yl)benzamide;
4-methyl-3-(9H-pyrimido[4,5-b]indol-7-yl)-N-[3-(trifluoromethoxy)phenyl]benzamide;
N-(2,5-difluorobenzyl)-4-methyl-3-(9H-pyrimido[4,5-b]indol-7-yl)benzamide;
4-methyl-3-(9H-pyrimido[4,5-b]indol-7-yl)-N-[3-(trifluoromethyl)benzyl]benzamide;
4-methyl-N-(5-methyl-1,3-thiazol-2-yl)-3-(9H-pyrimido[4,5-b]indol-7-yl)benzamide;
4-chloro-3-(9H-pyrimido[4,5-b]indol-7-yl)-N-[3-(trifluoromethyl)phenyl]benzamide;
3-(9H-pyrimido[4,5-b]indol-7-yl)-N-[3-(trifluoromethyl)phenyl]benzamide;
4-methoxy-3-(9H-pyrimido[4,5-b]indol-7-yl)-N-[3-(trifluoromethyl)phenyl]benzamide;
4-methyl-3-(9H-pyrimido[4,5-b]indol-7-yl)-N-[5-(trifluoromethyl)-1,3,4-thiadiazol-2-
yl]benzamide;
4-methyl-N-[3-(4-methyl-1H-imidazol-1-yl)-5-(trifluoromethyl)phenyl]-3-(9H-
pyrimido[4,5-b]indol-7-yl)benzamide;
4-methyl-N-[(1R)-1-phenylethyl]-3-(9H-pyrimido[4,5-b]indol-7-yl)benzamide;
4-methyl-N-[(1S)-1-phenylethyl]-3-(9H-pyrimido[4,5-b]indol-7-yl)benzamide;
4-methyl-N-[3-(pentafluoro-λ6)-sulfanyl)phenyl]-3-(9H-pyrimido[4,5-b]indol-7-
yl)benzamide;
4-methyl-3-(9H-pyrimido[4,5-b]indol-7-yl)-N-[5-(trifluoromethyl)pyridin-3-
yl]benzamide;
N-[3-(2-amino-9H-pyrimido[4,5-b]indol-7-yl)-4-methylphenyl]-3-
(trifluoromethyl)benzamide;
4-methyl-3-(9H-pyrido[2,3-b]indol-7-yl)-N-[3-(trifluoromethyl)phenyl]benzamide;
4-methyl-3-(9H-pyrido[3′,2′:4,5]pyrrolo[2,3-d]pyrimidin-7-yl)-N-[3-
(trifluoromethyl)phenyl]benzamide;
N-[6-methyl-5-(9H-pyrimido[4,5-b]indol-7-yl)pyridin-3-yl]-3-
(trifluoromethyl)benzamide;
3-fluoro-N-[6-methyl-5-(9H-pyrimido[4,5-b]indol-7-yl)pyridin-3-yl]-5-
(trifluoromethyl)benzamide;
3-(1H-imidazol-1-yl)-N-[6-methyl-5-(9H-pyrimido[4,5-b]indol-7-yl)pyridin-3-yl]-5-
(trifluoromethyl)benzamide;
N-[6-methyl-5-(9H-pyrimido[4,5-1)]indol-7-yl)pyridin-3-yl]-3-(1H-1,2,4-triazol-1-yl)-5-
(trifluoromethyl)benzamide;
3-(4-formyl-1H-imidazol-1-yl)-N-[6-methyl-5-(9H-pyrimido[4,5-1)]indol-7-yl)pyridin-3-
yl]-5-(trifluoromethyl)benzamide;
3-[4-(hydroxymethyl)-1H-imidazol-1-yl]-N-[6-methyl-5-(9H-pyrimido[4,5-b]indol-7-
yl)pyridin-3-yl]-5-(trifluoromethyl)benzamide;
3-[2-(dimethylamino)ethyl]amino-N-[6-methyl-5-(9H-pyrimido[4,5-b]indol-7-yl)pyridin-
3-yl]-5-(trifluoromethyl)benzamide;
3-[3-(dimethylamino)propyl]amino-N-[6-methyl-5-(9H-pyrimido[4,5-b]indol-7-
yl)pyridin-3-yl]-5-(trifluoromethyl)benzamide
3-(4-(2-(4-(1H-imidazol-1-yl)phenylamino)pyrimidin-4-yl)-1H-pyrazol-1-yl)-4-(1-(2,4-
difluorobenzoyl)piperidin-4-yl)butanenitrile;
4-(1-(2,4-difluorobenzoyl)piperidin-4-yl)-3-(4-(2-(4-(piperazin-1-
yl)phenylamino)pyrimidin-4-yl)-1H-pyrazol-1-yl)butanenitrile;
4-(1-(2,4-difluorobenzoyl)piperidin-4-yl)-3-(4-(2-(4-methoxyphenylamino)pyrimidin-4-
yl)-1H-pyrazol-1-yl)butanenitrile;
4-(1-(2,4-difluorobenzoyl)piperidin-4-yl)-3-(4-(2-(phenylamino)pyrimidin-4-yl)-1H-
pyrazol-1-yl)butanenitrile;
4-(1-(2,4-difluorobenzoyl)piperidin-4-yl)-3-(4-(2-(4-morpholinophenylamino)pyrimidin-
4-yl)-1H-pyrazol-1-yl)butanenitrile;
3-(4-(2-(4-(1H-pyrazol-1-yl)phenylamino)pyrimidin-4-yl)-1H-pyrazol-1-yl)-4-(1-(2,4-
difluorobenzoyl)piperidin-4-yl)butanenitrile;
4-(1-(2,4-difluorobenzoyl)piperidin-4-yl)-3-(4-(2-(3-(oxazol-5-
yl)phenylamino)pyrimidin-4-yl)-1H-pyrazol-1-yl)butanenitrile;
4-(1-(2,4-difluorobenzoyl)piperidin-4-yl)-3-(4-(2-(1-methyl-1H-pyrazol-3-
ylamino)pyrimidin-4-yl)-1H-pyrazol-1-yl)butanenitrile;
4-(1-(2,4-difluorobenzoyl)piperidin-4-yl)-3-(4-(2-(4-phenoxyphenylamino)pyrimidin-4-
yl)-1H-pyrazol-1-yl)butanenitrile;
2-(4-(4-(2-(4-(1H-pyrazol-1-yl)phenylamino)pyrimidin-4-yl)-1H-pyrazol-1-yl)-1-
(isoxazole-5-carbonyl)piperidin-4-yl)acetonitrile;
2-(1-(isoxazole-5-carbonyl)-4-(4-(2-(3-(oxazol-5-yl)phenylamino)pyrimidin-4-yl)-1H-
pyrazol-1-yl)piperidin-4-yl)acetonitrile;
2-(4-(4-(2-(3-(1H-tetrazol-5-yl)phenylamino)pyrimidin-4-yl)-1H-pyrazol-1-yl)-1-
(isoxazole-5-carbonyl)piperidin-4-yl)acetonitrile;
2-(1-(isoxazole-5-carbonyl)-4-(4-(2-(4-(morpholinosulfonyl)phenylamino)pyrimidin-4-
yl)-1H-pyrazol-1-yl)piperidin-4-yl)acetonitrile;
2-(1-(isoxazole-5-carbonyl)-4-(4-(2-(6-methoxypyridin-3-ylamino)pyrimidin-4-yl)-1H-
pyrazol-1-yl)piperidin-4-yl)acetonitrile;
2-(3-(4-(2-(4-(1H-pyrazol-1-yl)phenylamino)pyrimidin-4-yl)-1H-pyrazol-1-yl)-1-
(cyclopropylsulfonyl)azetidin-3-yl)acetonitrile;
2-(1-(cyclopropylsulfonyl)-3-(4-(2-(3-(oxazol-5-yl)phenylamino)pyrimidin-4-yl)-1H-
pyrazol-1-y)azetidin-3-yl)acetonitrile;
N-(4-(4-(1-(3-(cyanomethyl)-1-(cyclopropylsulfonyl)azetidin-3-yl)-1H-pyrazol-4-
yl)pyrimidin-2-ylamino)phenyl)acetamide;
2-(1-(cyclopropylsulfonyl)-3-(4-(2-(3-(2-methylpyrimidin-4-yl)phenylamino)pyrimidin-
4-yl)-1H-pyrazol-1-yl)azetidin-3-yl)acetonitrile;
2-(1-(cyclopropylsulfonyl)-3-(4-(2-(4-(oxazol-5-yl)phenylamino)pyrimidin-4-yl)-1H-
pyrazol-1-yl)azetidin-3-yl)acetonitrile;
3-(4-(2-(4-morpholinophenylamino)pyrimidin-4-yl)-1H-pyrazol-1-yl)-3-(piperidin-4-
yl)propanenitrile;
3-(1-(5-fluoropyrimidin-2-yl)piperidin-4-yl)-3-(4-(2-(4-
morpholinophenylamino)pyrimidin-4-yl)-1H-pyrazol-1-yl)propanenitrile;
3-(1-(methylsulfonyl)piperidin-4-yl)-3-(4-(2-(4-morpholinophenylamino)pyrimidin-4-
yl)-1H-pyrazol-1-yl)propanenitrile;
3-(4-(2-(4-morpholinophenylamino)pyrimidin-4-yl)-1H-pyrazol-1-yl)-3-(1-
(phenylsulfonyl)piperidin-4-yl)propanenitrile;
3-(1-acetylpiperidin-4-yl)-3-(4-(2-(4-morpholinophenylamino)pyrimidin-4-yl)-1H-
pyrazol-1-yl)propanenitrile;
3-(1-benzoylpiperidin-4-yl)-3-(4-(2-(4-morpholinophenylamino)pyrimidin-4-yl)-1H-
pyrazol-1-yl)propanenitrile;
2-(4-(4-(2-(4-(1H-pyrazol-1-yl)phenylamino)pyrimidin-4-yl)-1H-pyrazol-1-yl)-1-
(cyclopropylsulfonyl)piperidin-4-yl)acetonitrile;
2-(1-(cyclopropylsulfonyl)-4-(4-(2-(4-morpholinophenylamino)pyrimidin-4-yl)-1H-
pyrazol-1-yl)piperidin-4-yl)acetonitrile;
4-(4-(1-(4-(cyanomethyl)-1-(cyclopropylsulfonyl)piperidin-4-yl)-1H-pyrazol-4-
yl)pyrimidin-2-ylamino)benzamide;
4-(4-(1-(4-(cyanomethyl)-1-(cyclopropylsulfonyl)piperidin-4-yl)-1H-pyrazol-4-
yl)pyrimidin-2-ylamino)-N-(2-hydroxyethyl)benzamide;
4-(4-(1-(4-(cyanomethyl)-1-(cyclopropylsulfonyl)piperidin-4-yl)-1H-pyrazol-4-
yl)pyrimidin-2-ylamino)-N,N-dimethylbenzamide;
4-(4-(1-(2-cyano-1-cyclopentylethyl)-1H-pyrazol-4-yl)pyrimidin-2-ylamino)benzamide;
3-(4-(2-(4-(1H-pyrazol-1-yl)phenylamino)pyrimidin-4-yl)-1H-pyrazol-1-yl)-3-
cyclopentylpropanenitrile;
3-cyclopentyl-3-(4-(2-(4-morpholinophenylamino)pyrimidin-4-yl)-1H-pyrazol-1-
yl)propanenitrile;
3-cyclopentyl-3-(4-(2-(phenylamino)pyrimidin-4-yl)-1H-pyrazol-1-yl)propanenitrile;
3-cyclopentyl-3-(4-(2-(3-(oxazol-5-yl)phenylamino)pyrimidin-4-yl)-1H-pyrazol-1-
yl)propanenitrile;
3-cyclopentyl-3-(4-(2-(4-methoxyphenylamino)pyrimidin-4-yl)-1H-pyrazol-1-
yl)propanenitrile;
N-(4-(4-(1-(2-cyano-1-cyclopentylethyl)-1H-pyrazol-4-yl)pyrimidin-2-
ylamino)phenyl)acetamide;
4-(4-(1-(2-cyano-1-cyclopentylethyl)-1H-pyrazol-4-yl)pyrimidin-2-ylamino)-N,N-
dimethylbenzamide;
3-cyclopentyl-3-(4-(2-(4-(piperazin-1-yl)phenylamino)pyrimidin-4-yl)-1H-pyrazol-1-
yl)propanenitrile;
4-(1-(ethylsulfonyl)piperidin-4-yl)-3-(4-(2-(4-morpholinophenylamino)pyrimidin-4-yl)-
1H-pyrazol-1-yl)butanenitrile;
3-(4-(2-(4-(1H-pyrazol-1-yl)phenylamino)pyrimidin-4-yl)-1H-pyrazol-1-yl)-4-(1-
(ethylsulfonyl)piperidin-4-yl)butanenitrile;
4-(1-(ethylsulfonyl)piperidin-4-yl)-3-(4-(2-(phenylamino)pyrimidin-4-yl)-1H-pyrazol-1-
yl)butanenitrile;
N-(4-(4-(1-(1-cyano-3-(1-(1-methyl-1H-pyrazol-3-ylsulfonyl)piperidin-4-yl)propan-2-
yl)-1H-pyrazol-4-yl)pyrimidin-2-ylamino)phenyl)acetamide;
4-(4-(1-(1-cyano-3-(1-(ethylsulfonyl)piperidin-4-yl)propan-2-yl)-1H-pyrazol-4-
yl)pyrimidin-2-ylamino)-N,N-dimethylbenzamide;
4-(4-(1-(1-cyano-3-(1-(1-methyl-1H-pyrazol-3-ylsulfonyl)piperidin-4-yl)propan-2-yl)-
1H-pyrazol-4-yl)pyrimidin-2-ylamino)benzamide;
4-(1-(ethylsulfonyl)piperidin-4-yl)-3-(4-(5-methyl-2-(4-
morpholinophenylamino)pyrimidin-4-yl)-1H-pyrazol-1-yl)butanenitrile;
3-(4-(2-(4-(1H-pyrazol-1-yl)phenylamino)-5-methylpyrimidin-4-yl)-1H-pyrazol-1-yl)-4-
(1-(ethylsulfonyl)piperidin-4-yl)butanenitrile;
4-(1-(ethylsulfonyl)piperidin-4-yl)-3-(4-(5-methyl-2-(phenylamino)pyrimidin-4-yl)-1H-
pyrazol-1-yl)butanenitrile;
N-(4-(4-(1-(1-cyano-3-(1-(1-methyl-1H-pyrazol-3-ylsulfonyl)piperidin-4-yl)propan-2-
yl)-1H-pyrazol-4-yl)-5-methylpyrimidin-2-ylamino)phenyl)acetamide;
4-(4-(1-(1-cyano-3-(1-(ethylsulfonyl)piperidin-4-yl)propan-2-yl)-1H-pyrazol-4-yl)-5-
methylpyrimidin-2-ylamino)-N,N-dimethylbenzamide;
4-(4-(1-(1-cyano-3-(1-(1-methyl-1H-pyrazol-3-ylsulfonyl)piperidin-4-yl)propan-2-yl)-
1H-pyrazol-4-yl)-5-methylpyrimidin-2-ylamino)benzamide;
3-cyclopentyl-3-(4-(2-(4-(4-(methylsulfonyl)piperazin-1-yl)phenylamino)pyrimidin-4-
yl)-1H-pyrazol-1-yl)propanenitrile;
4-(1-(1-methyl-1H-pyrazol-3-ylsulfonyl)piperidin-4-yl)-3-(4-(2-(4-
morpholinophenylamino)pyrimidin-4-yl)-1H-pyrazol-1-yl)butanenitrile;
3-(4-(2-(4-(1H-pyrazol-1-yl)phenylamino)pyrimidin-4-yl)-1H-pyrazol-1-yl)-4-(1-(1-
methyl-1H-pyrazol-3-ylsulfonyl)piperidin-4-yl)butanenitrile;
4-(1-(1-methyl-1H-pyrazol-3-ylsulfonyl)piperidin-4-yl)-3-(4-(2-
(phenylamino)pyrimidin-4-yl)-1H-pyrazol-1-yl)butanenitrile;
4-(4-(1-(1-cyano-3-(1-(1-methyl-1H-pyrazol-3-ylsulfonyl)piperidin-4-yl)propan-2-yl)-
1H-pyrazol-4-yl)pyrimidin-2-ylamino)-N,N-dimethylbenzamide;
4-(4-(1-(1-cyano-3-(1-(2,4-difluorobenzoyl)piperidin-4-yl)propan-2-yl)-1H-pyrazol-4-
yl)pyrimidin-2-ylamino)-N,N-dimethylbenzamide;
N-(4-(4-(1-(1-cyano-3-(1-(2,4-difluorobenzoyl)piperidin-4-yl)propan-2-yl)-1H-pyrazol-
4-yl)pyrimidin-2-ylamino)phenyl)acetamide;
4-(1-(1-methyl-1H-pyrazol-3-ylsulfonyl)piperidin-4-yl)-3-(4-(5-methyl-2-(4-
morpholinophenylamino)pyrimidin-4-yl)-1H-pyrazol-1-yl)butanenitrile;
4-(1-(2,4-difluorobenzoyl)piperidin-4-yl)-3-(4-(5-methyl-2-(4-
morpholinophenylamino)pyrimidin-4-yl)-1H-pyrazol-1-yl)butanenitrile;
3-(4-(2-(4-(1H-pyrazol-1-yl)phenylamino)-5-methylpyrimidin-4-yl)-1H-pyrazol-1-yl)-4-
(1-(1-methyl-1H-pyrazol-3-ylsulfonyl)piperidin-4-yl)butanenitrile;
3-(4-(2-(4-(1H-pyrazol-1-yl)phenylamino)-5-methylpyrimidin-4-yl)-1H-pyrazol-1-yl)-4-
(1-(2,4-difluorobenzoyl)piperidin-4-yl)butanenitrile;
4-(1-(1-methyl-1H-pyrazol-3-ylsulfonyl)piperidin-4-yl)-3-(4-(5-methyl-2-
(phenylamino)pyrimidin-4-yl)-1H-pyrazol-1-yl)butanenitrile;
4-(1-(2,4-difluorobenzoyl)piperidin-4-yl)-3-(4-(5-methyl-2-(phenylamino)pyrimidin-4-
yl)-1H-pyrazol-1-yl)butanenitrile;
4-(4-(1-(1-cyano-3-(1-(1-methyl-1H-pyrazol-3-ylsulfonyl)piperidin-4-yl)propan-2-yl)-
1H-pyrazol-4-yl)-5-methylpyrimidin-2-ylamino)-N,N-dimethylbenzamide;
4-(4-(1-(1-cyano-3-(1-(2,4-difluorobenzoyl)piperidin-4-yl)propan-2-yl)-1H-pyrazol-4-
yl)-5-methylpyrimidin-2-ylamino)-N,N-dimethylbenzamide;
N-(4-(4-(1-(1-cyano-3-(1-(2,4-difluorobenzoyl)piperidin-4-yl)propan-2-yl)-1H-pyrazol-
4-yl)-5-methylpyrimidin-2-ylamino)phenyl)acetamide;
4-(4-(1-(2-cyano-1-cyclopentylethyl)-1H-pyrazol-4-yl)-5-methylpyrimidin-2-
ylamino)benzamide;
3-(4-(2-(4-(1H-pyrazol-1-yl)phenylamino)-5-methylpyrimidin-4-yl)-1H-pyrazol-1-yl)-3-
cyclopentylpropanenitrile;
3-cyclopentyl-3-(4-(5-methyl-2-(4-morpholinophenylamino)pyrimidin-4-yl)-1H-pyrazol-
1-yl)propanenitrile;
3-cyclopentyl-3-(4-(5-methyl-2-(phenylamino)pyrimidin-4-yl)-1H-pyrazol-1-
yl)propanenitrile;
3-cyclopentyl-3-(4-(5-methyl-2-(4-(oxazol-5-yl)phenylamino)pyrimidin-4-yl)-1H-
pyrazol-1-yl)propanenitrile;
3-cyclopentyl-3-(4-(2-(4-methoxyphenylamino)-5-methylpyrimidin-4-yl)-1H-pyrazol-1-
yl)propanenitrile;
3-cyclopentyl-3-(4-(5-methyl-2-(4-(piperazin-1-yl)phenylamino)pyrimidin-4-yl)-1H-
pyrazol-1-yl)propanenitrile;
3-cyclopentyl-3-(4-(2-(4-(diethylamino)phenylamino)-5-methylpyrimidin-4-yl)-1H-
pyrazol-1-yl)propanenitrile;
3-cyclopentyl-3-(4-(2-(4-(ethyl(3-hydroxypropyl)amino)phenylamino)-5-
methylpyrimidin-4-yl)-1H-pyrazol-1-yl)propanenitrile;
4-(4-(1-(2-cyano-1-cyclopentylethyl)-1H-pyrazol-4-yl)-5-methylpyrimidin-2-
ylamino)benzoic acid;
3-cyclopentyl-3-(4-(5-methyl-2-(4-nitrophenylamino)pyrimidin-4-yl)-1H-pyrazol-1-
yl)propanenitrile;
4-(4-(1-(2-cyano-1-cyclopentylethyl)-1H-pyrazol-4-yl)-5-methylpyrimidin-2-ylamino)-
N-(2-hydroxyethyl)benzamide;
3-cyclopentyl-3-(4-(5-methyl-2-(3-(oxazol-5-yl)phenylamino)pyrimidin-4-yl)-1H-
pyrazol-1-yl)propanenitrile;
3-(4-(2-(4-aminophenylamino)-5-methylpyrimidin-4-yl)-1H-pyrazol-1-yl)-3-
cyclopentylpropanenitrile;
4-(4-(1-(2-cyano-1-cyclopentylethyl)-1H-pyrazol-4-yl)-5-methylpyrimidin-2-ylamino)-
N-methylbenzamide;
4-(4-(1-(2-cyano-1-cyclopentylethyl)-1H-pyrazol-4-yl)-5-methylpyrimidin-2-ylamino)-
N-(1-methoxypropan-2-yl)benzamide;
3-cyclopentyl-3-(4-(2-(4-(4-hydroxypiperidine-1-carbonyl)phenylamino)-5-
methylpyrimidin-4-yl)-1H-pyrazol-1-yl)propanenitrile;
N-(4-(4-(1-(2-cyano-1-cyclopentylethyl)-1H-pyrazol-4-yl)-5-methylpyrimidin-2-
ylamino)phenyl)methanesulfonamide;
Methyl4-(4-(1-(2-cyano-1-cyclopentylethyl)-1H-pyrazol-4-yl)-5-methylpyrimidin-2-
ylamino)phenylcarbamate;
N-(4-(4-(1-(2-cyano-1-cyclopentylethyl)-1H-pyrazol-4-yl)-5-methylpyrimidin-2-
ylamino)phenyl)-2-(pyrrolidin-1-yl)acetamide;
3-(4-(2-(4-(3-oxomorpholino)phenylamino)pyrimidin-4-yl)-1H-pyrazol-1-yl)-4-
(piperidin-4-yl)butanenitrile;
2-(1-(cyclopropylsulfonyl)-3-(4-(2-(4-morpholinophenylamino)pyrimidin-4-yl)-1H-
pyrazol-1-yl)azetidin-3-yl)acetonitrile;
2-(1-(isoxazole-5-carbonyl)-4-(4-(2-(4-morpholinophenylamino)pyrimidin-4-yl)-1H-
pyrazol-1-yl)piperidin-4-yl)acetonitrile;
4-(1-(methylsulfonyl)piperidin-4-yl)-3-(4-(2-(4-(3-
oxomorpholino)phenylamino)pyrimidin-4-yl)-1H-pyrazol-1-yl)butanenitrile;
4-(1-(ethylsulfonyl)piperidin-4-yl)-3-(4-(2-(4-(3-
oxomorpholino)phenylamino)pyrimidin-4-yl)-1H-pyrazol-1-yl)butanenitrile;
4-(1-(cyclopropylsulfonyl)piperidin-4-yl)-3-(4-(2-(4-(3-
oxomorpholine)phenylamino)pyrimidin-4-yl)-1H-pyrazol-1-yl)butanenitrile;
3-cyclopentyl-3-(4-(2-(4-(3-oxomorpholino)phenylamino)pyrimidin-4-yl)-1H-pyrazol-1-
yl)propanenitrile;
3-cyclopentyl-3-(4-(2-(3-(2-methylpyrimidin-4-yl)phenylamino)pyrimidin-4-yl)-1H-
pyrazol-1-yl)propanenitrile;
3-(4-(1-(2-cyano-1-cyclopentylethyl)-1H-pyrazol-4-yl)pyrimidin-2-ylamino)benzoic
acid;
3-cyclopentyl-3-(4-(5-methoxy-2-(4-morpholinophenylamino)pyrimidin-4-yl)-1H-
pyrazol-1-yl)propanenitrile;
3-(4-(2-(4-(1H-pyrazol-1-yl)phenylamino)-5-methoxypyrimidin-4-yl)-1H-pyrazol-1-yl)-
3-cyclopentylpropanenitrile;
N-(4-(4-(1-(2-cyano-1-cyclopentylethyl)-1H-pyrazol-4-yl)-5-methoxypyrimidin-2-
ylamino)phenyl)acetamide;
4-(4-(1-(2-cyano-1-cyclopentylethyl)-1H-pyrazol-4-yl)-5-methoxypyrimidin-2-ylamino)-
N,N-dimethylbenzamide;
3-cyclopentyl-3-(4-(2-(4-(2-oxopiperidin-1-yl)phenylamino)pyrimidin-4-yl)-1H-pyrazol-
1-yl)propanenitrile;
3-cyclopentyl-3-(4-(2-(4-(2-oxo-1,3-oxazinan-3-yl)phenylamino)pyrimidin-4-yl)-1H-
pyrazol-1-yl)propanenitrile;
3-cyclopentyl-3-(4-(2-(4-(2-oxooxazolidin-3-yl)phenylamino)pyrimidin-4-yl)-1H-
pyrazol-1-yl)propanenitrile;
3-(4-(2-(3-aminophenylamino)pyrimidin-4-yl)-1H-pyrazol-1-yl)-3-
cyclopentylpropanenitrile;
3-(4-(1-(2-cyano-1-cyclopentylethyl)-1H-pyrazol-4-yl)pyrimidin-2-ylamino)-N-
methylbenzamide;
3-(4-(1-(2-cyano-1-cyclopentylethyl)-1H-pyrazol-4-yl)pyrimidin-2-ylamino)-N,N-
dimethylbenzamide;
3-cyclopentyl-3-(4-(2-(3-(4-hydroxypiperidine-1-carbonyl)phenylamino)pyrimidin-4-yl)-
1H-pyrazol-1-yl)propanenitrile;
3-(4-(1-(2-cyano-1-cyclopentylethyl)-1H-pyrazol-4-yl)pyrimidin-2-ylamino)-N-(2-
hydroxyethyl)benzamide;
3-(4-(1-(2-cyano-1-cyclopentylethyl)-1H-pyrazol-4-yl)pyrimidin-2-ylamino)-N-(1-
methoxypropan-2-yl)benzamide;
N-(3-(4-(1-(2-cyano-1-cyclopentylethyl)-1H-pyrazol-4-yl)pyrimidin-2-
ylamino)phenyl)ethanesulfonamide;
N-(3-(4-(1-(2-cyano-1-cyclopentylethyl)-1H-pyrazol-4-yl)pyrimidin-2-
ylamino)phenyl)methanesulfonamide;
methyl3-(4-(1-(2-cyano-1-cyclopentylethyl)-1H-pyrazol-4-yl)pyrimidin-2-
ylamino)phenylcarbamate;
N-(3-(4-(1-(2-cyano-1-cyclopentylethyl)-1H-pyrazol-4-yl)pyrimidin-2-
ylamino)phenyl)acetamide;
N-(3-(4-(1-(2-cyano-1-cyclopentylethyl)-1H-pyrazol-4-yl)pyrimidin-2-ylamino)phenyl)-
2-(pyrrolidin-1-yl)acetamide;
4-(4-(1-(2-cyano-1-cyclopentylethyl)-1H-pyrazol-4-yl)pyrimidin-2-ylamino)benzoic
acid;
3-cyclopentyl-3-(4-(2-(4-(4-methylpiperazine-1-carbonyl)phenylamino)pyrimidin-4-yl)-
1H-pyrazol-1-yl)propanenitrile;
3-cyclopentyl-3-(4-(2-(4-(4-(2-hydroxyethyl)piperazine-1-
carbonyl)phenylamino)pyrimidin-4-yl)-1H-pyrazol-1-yl)propanenitrile;
3-cyclopentyl-3-(4-(2-(4-(pyrrolidine-1-carbonyl)phenylamino)pyrimidin-4-yl)-1H-
pyrazol-1-yl)propanenitrile;
3-cyclopentyl-3-(4-(2-(4-(3-oxopiperazine-1-carbonyl)phenylamino)pyrimidin-4-yl)-1H-
pyrazol-1-yl)propanenitrile;
3-cyclopentyl-3-(4-(2-(4-(4-hydroxypiperidine-1-carbonyl)phenylamino)pyrimidin-4-yl)-
1H-pyrazol-1-yl)propanenitrile;
4-(4-(1-(2-cyano-1-cyclopentylethyl)-1H-pyrazol-4-yl)pyrimidin-2-ylamino)-N-
(cyclopropylmethyl)-N-propylbenzamide;
4-(4-(1-(2-cyano-1-cyclopentylethyl)-1H-pyrazol-4-yl)pyrimidin-2-ylamino)-N-
(cyclopropylmethyl)benzamide;
3-cyclopentyl-3-(4-(2-(4-(3-hydroxypyrrolidine-1-carbonyl)phenylamino)pyrimidin-4-
yl)-1H-pyrazol-1-yl)propanenitrile;
3-(4-(2-(4-(azetidine-1-carbonyl)phenylamino)pyrimidin-4-yl)-1H-pyrazol-1-yl)-3-
cyclopentylpropanenitrile;
3-cyclopentyl-3-(4-(2-(4-(2-oxopyrrolidin-1-yl)phenylamino)pyrimidin-4-yl)-1H-
pyrazol-1-yl)propanenitrile;
3-cyclopentyl-3-(4-(5-methoxy-2-(4-(2-oxopyrrolidin-1-yl)phenylamino)pyrimidin-4-yl)-
1H-pyrazol-1-yl)propanenitrile;
3-cyclopentyl-3-(4-(5-methoxy-2-(4-(oxazol-5-yl)phenylamino)pyrimidin-4-yl)-1H-
pyrazol-1-yl)propanenitrile;
3-cyclopentyl-3-(4-(5-methoxy-2-(3-(oxazol-5-yl)phenylamino)pyrimidin-4-yl)-1H-
pyrazol-1-yl)propanenitrile;
3-cyclopentyl-3-(4-(5-methoxy-2-(4-(3-oxomorpholino)phenylamino)pyrimidin-4-yl)-
1H-pyrazol-1-yl)propanenitrile;
3-cyclopentyl-3-(4-(5-methoxy-2-(3-(2-methylpyrimidin-4-yl)phenylamino)pyrimidin-4-
yl)-1H-pyrazol-1-yl)propanenitrile;
3-cyclopentyl-3-(4-(5-methoxy-2-(4-(2-oxopiperidin-1-yl)phenylamino)pyrimidin-4-yl)-
1H-pyrazol-1-yl)propanenitrile;
3-cyclopentyl-3-(4-(5-methoxy-2-(4-(2-oxooxazolidin-3-yl)phenylamino)pyrimidin-4-
yl)-1H-pyrazol-1-yl)propanenitrile;
3-cyclopentyl-3-(4-(2-(3-(4-methylpiperazine-1-carbonyl)phenylamino)pyrimidin-4-yl)-
1H-pyrazol-1-yl)propanenitrile;
3-cyclopentyl-3-(4-(2-(3-(4-(2-hydroxyethyl)piperazine-1-
carbonyl)phenylamino)pyrimidin-4-yl)-1H-pyrazol-1-yl)propanenitrile;
3-cyclopentyl-3-(4-(2-(3-(pyrrolidine-1-carbonyl)phenylamino)pyrimidin-4-yl)-1H-
pyrazol-1-yl)propanenitrile;
3-cyclopentyl-3-(4-(2-(3-(3-oxopiperazine-1-carbonyl)phenylamino)pyrimidin-4-yl)-1H-
pyrazol-1-yl)propanenitrile;
3-cyclopentyl-3-(4-(2-(3-(3-hydroxypyrrolidine-1-carbonyl)phenylamino)pyrimidin-4-
yl)-1H-pyrazol-1-yl)propanenitrile;
3-(4-(2-(3-(azetidine-1-carbonyl)phenylamino)pyrimidin-4-yl)-1H-pyrazol-1-yl)-3-
cyclopentylpropanenitrile;
3-(4-(2-(3-(4-acetylpiperazine-1-carbonyl)phenylamino)pyrimidin-4-yl)-1H-pyrazol-
1yl)-3-cyclopentylpropanenitrile;
3-cyclopentyl-3-(4-(2-(3-(4-(pyridin-3-ylmethyl)piperidine-1-
carbonyl)phenylamino)pyrimidin-4-yl)-1H-pyrazol-1-yl)propanenitrile;
3-(4-(1-(2-cyano-1-cyclopentylethyl)-1H-pyrazol-4-yl)pyrimidin-2-ylamino)-N-(1-(3-
methoxyphenyl)ethyl)benzamide;
3-(4-(1-(2-cyano-1-cyclopentylethyl)-1H-pyrazol-4-yl)pyrimidin-2-ylamino)-N-(pyridin-
3-ylmethyl)benzamide;
3-cyclopentyl-3-(4-(2-(3-(morpholine-4-carbonyl)phenylamino)pyrimidin-4-yl)-1H-
pyrazol-1-yl)propanenitrile;
3-(4-(1-(2-cyano-1-cyclopentylethyl)-1H-pyrazol-4-yl)pyrimidin-2-ylamino)-N-((5-
methylisoxazol-3-yl)methyl)benzamide;
3-(4-(1-(2-cyano-1-cyclopentylethyl)-1H-pyrazol-4-yl)pyrimidin-2-ylamino)-N-(2-(1-
methylpyrrolidin-2-yl)ethyl)benzamide;
3-cyclopentyl-3-(4-(2-(3-(4-hydroxy-4-phenylpiperidine-1-
carbonyl)phenylamino)pyrimidin-4-yl)-1H-pyrazol-1-yl)propanenitrile;
3-(4-(2-(3-(4-benzyl-4-hydroxypiperidine-1-carbonyl)phenylamino)pyrimidin-4-yl)-1H-
pyrazol-1-yl)-3-cyclopentylpropanenitrile;
3-cyclopentyl-3-(4-(2-(3-(3-(pyridin-2-yl)pyrrolidine-1-
carbonyl)phenylamino)pyrimidin-4-yl)-1H-pyrazol-1-yl)propanenitrile;
3-(4-(1-(2-cyano-1-cyclopentylethyl)-1H-pyrazol-4-yl)pyrimidin-2-ylamino)-N-
((tetrahydro-2H-pyran-4-yl)methyl)benzamide;
3-(4-(1-(2-cyano-1-cyclopentylethyl)-1H-pyrazol-4-yl)pyrimidin-2-ylamino)-N-(1-
methylpiperidin-4-yl)benzamide;
3-cyclopentyl-3-(4-(2-(3-(4-phenylpiperidine-1-carbonyl)phenylamino)pyrimidin-4-yl)-
1H-pyrazol-1-yl)propanenitrile;
3-(4-(1-(2-cyano-1-cyclopentylethyl)-1H-pyrazol-4-yl)pyrimidin-2-ylamino)-N-(1-
(pyridin-2-yl)ethyl)benzamide;
3-cyclopentyl-3-(4-(2-(3-(3-(3-fluorophenyl)pyrrolidine-1-
carbonyl)phenylamino)pyrimidin-4-yl)-1H-pyrazol-1-yl)propanenitrile;
N-((3R)-1-(3-(4-(1-(2-cyano-1-cyclopentylethyl)-1H-pyrazol-4-yl)pyrimidin-2-
ylamino)benzoyl)pyrrolidin-3-yl)acetamide;
3-(4-(1-(2-cyano-1-cyclopentylethyl)-1H-pyrazol-4-yl)pyrimidin-2-ylamino)-N-(2-(2-
oxoimidazolidin-1-yl)ethyl)benzamide;
3-cyclopentyl-3-(4-(2-(3-(4-(pyrimidin-2-yl)piperazine-1-
carbonyl)phenylamino)pyrimidin-4-yl)-1H-pyrazol-1-yl)propanenitrile;
3-(4-(1-(2-cyano-1-cyclopentylethyl)-1H-pyrazol-4-yl)pyrimidin-2-ylamino)-N-(2-
(pyridin-3-yl)ethyl)benzamide;
3-cyclopentyl-3-(4-(2-(3-(2-(methoxymethyl)pyrrolidine-1-
carbonyl)phenylamino)pyrimidin-4-yl)-1H-pyrazol-1-yl)propanenitrile;
3-(4-(1-(2-cyano-1-cyclopentylethyl)-1H-pyrazol-4-yl)pyrimidin-2-ylamino)-N-(2-
methoxybenzyl)benzamide;
3-cyclopentyl-3-(4-(2-(3-(4-phenoxypiperidine-1-carbonyl)phenylamino)pyrimidin-4-
yl)-1H-pyrazol-1-yl)propanenitrile;
3-(4-(1-(2-cyano-1-cyclopentylethyl)-1H-pyrazol-4-yl)pyrimidin-2-ylamino)-N-(1-
(hydroxymethyl)cyclopentyl)benzamide;
4-(4-(3-(4-(1-(2-cyano-1-cyclopentylethyl)-1H-pyrazol-4-yl)pyrimidin-2-
ylamino)benzoyl)piperazin-1-yl)benzonitrile;
N-(1-benzylpyrrolidin-3-yl)-3-(4-(1-(2-cyano-1-cyclopentylethyl)-1H-pyrazol-4-
yl)pyrimidin-2-ylamino)benzamide;
3-cyclopentyl-3-(4-(2-(3-(4-phenylpiperazine-1-carbonyl)phenylamino)pyrimidin-4-yl)-
1H-pyrazol-1-yl)propanenitrile;
3-cyclopentyl-3-(4-(2-(3-nitrophenylamino)pyrimidin-4-yl)-1H-pyrazol-1-
yl)propanenitrile;
3-cyclopentyl-3-(4-(2-(4-nitrophenylamino)pyrimidin-4-yl)-1H-pyrazol-1-
yl)propanenitrile;
3-cyclobutyl-3-(4-(2-(4-morpholinophenylamino)pyrimidin-4-yl)-1H-pyrazol-1-
yl)propanenitrile;
3-(4-(2-(4-aminophenylamino)pyrimidin-4-yl)-1H-pyrazol-1-yl)-3-
cyclopentylpropanenitrile;
3-(4-(2-(4-(1H-pyrazol-1-yl)phenylamino)pyrimidin-4-yl)-1H-pyrazol-1-yl)-3-
cyclobutylpropanenitrile;
3-cyclobutyl-3-(4-(2-(4-(2-oxopiperidin-1-yl)phenylamino)pyrimidin-4-yl)-1H-pyrazol-
1-yl)propanenitrile;
3-cyclobutyl-3-(4-(2-(4-(3-oxomorpholino)phenylamino)pyrimidin-4-yl)-1H-pyrazol-1-
yl)propanenitrile;
3-cyclobutyl-3-(4-(2-(3-(oxazol-5-yl)phenylamino)pyrimidin-4-yl)-1H-pyrazol-1-
yl)propanenitrile;
3-cyclopropyl-3-(4-(2-(4-morpholinophenylamino)pyrimidin-4-yl)-1H-pyrazol-1-
yl)propanenitrile;
3-(4-(2-(4-(1H-pyrazol-1-yl)phenylamino)pyrimidin-4-yl)-1H-pyrazol-1-yl)-3-
cyclopropylpropanenitrile;
3-cyclopropyl-3-(4-(2-(4-(2-oxopiperidin-1-yl)phenylamino)pyrimidin-4-yl)-1H-pyrazol-
1-yl)propanenitrile;
3-cyclopropyl-3-(4-(2-(4-(3-oxomorpholino)phenylamino)pyrimidin-4-yl)-1H-pyrazol-1-
yl)propanenitrile;
3-cyclopropyl-3-(4-(2-(3-(oxazol-5-yl)phenylamino)pyrimidin-4-yl)-1H-pyrazol-1-
yl)propanenitrile;
N-(4-(4-(1-(2-cyano-1-cyclopentylethyl)-1H-pyrazol-4-yl)pyrimidin-2-ylamino)phenyl)-
2,6-(cis)-dimethylmorpholine-4-sulfonamide;
N-(4-(4-(1-(2-cyano-1-cyclopentylethyl)-1H-pyrazol-4-yl)pyrimidin-2-
ylamino)phenyl)benzamide;
N-(4-(4-(1-(2-cyano-1-cyclopentylethyl)-1H-pyrazol-4-yl)pyrimidin-2-ylamino)phenyl)-
1-(methylsulfonyl)methanesulfonamide;
N-(4-(4-(1-(2-cyano-1-cyclopentylethyl)-1H-pyrazol-4-yl)pyrimidin-2-ylamino)phenyl)-
3,5-difluorobenzamide;
N′-(4-(4-(1-(2-cyano-1-cyclopentylethyl)-1H-pyrazol-4-yl)pyrimidin-2-ylamino)phenyl)-
N,N-dimethylsulfamide;
N-(4-(4-(1-(2-cyano-1-cyclopentylethyl)-1H-pyrazol-4-yl)pyrimidin-2-ylamino)phenyl)-
5-methylisoxazole-3-carboxamide;
N-(4-(4-(1-(2-cyano-1-cyclopentylethyl)-1H-pyrazol-4-yl)pyrimidin-2-
ylamino)phenyl)isoxazole-5-carboxamide;
N-(4-(4-(1-(2-cyano-1-cyclopentylethyl)-1H-pyrazol-4-yl)pyrimidin-2-ylamino)phenyl)-
3,5-dimethylisoxazole-4-carboxamide;
N-(4-(4-(1-(2-cyano-1-cyclopentylethyl)-1H-pyrazol-4-yl)pyrimidin-2-ylamino)phenyl)-
1-methyl-1H-pyrazole-3-sulfonamide;
N-(4-(4-(1-(2-cyano-1-cyclopentylethyl)-1H-pyrazol-4-yl)pyrimidin-2-ylamino)phenyl)-
2,5-difluorobenzamide;
3-cyclopentyl-3-(4-(2-(4-(1,1-dioxidoisothiazolidin-2-yl)phenyl)aminopyrimidin-4-yl)-
1H-pyrazol-1-yl)propanenitrile;
N-(4-(4-(1-(2-cyano-1-cyclopentylethyl)-1H-pyrazol-4-yl)pyrimidin-2-ylamino)phenyl)-
5-(2-methylthiazol-4-yl)thiophene-2-sulfonamide;
N-(4-(4-(1-(2-cyano-1-cyclopentylethyl)-1H-pyrazol-4-yl)pyrimidin-2-ylamino)phenyl)-
6-methylpyridine-2-sulfonamide;
N-(4-(4-(1-(2-cyano-1-cyclopentylethyl)-1H-pyrazol-4-yl)pyrimidin-2-ylamino)phenyl)-
5-(pyridin-2-yl)thiophene-2-sulfonamide;
5-chloro-N-(4-(4-(1-(2-cyano-1-cyclopentylethyl)-1H-pyrazol-4-yl)py-rimidin-2-
ylamino)phenyl)thiophene-2-sulfonamide;
N-(4-(4-(1-(2-cyano-1-cyclopentylethyl)-1H-pyrazol-4-yl)pyrimidin-2-ylamino)phenyl)-
6-morpholinopyridine-3-sulfonamide;
tetrahydrofuran-3-yl 4-(4-(1-(2-cyano-1-cyclopentylethyl)-1H-pyrazol-4-yl)pyrimidin-2-
ylamino)phenylcarbamate;
tetrahydrofuran-3-yl 3-(4-(1-(2-cyano-1-cyclopentylethyl)-1H-pyrazol-4-yl)pyrimidin-2-
ylamino)phenylcarbamate;
N-(3-(4-(1-(2-cyano-1-cyclopentylethyl)-1H-pyrazol-4-yl)pyrimidin-2-ylamino)phenyl)-
1-methyl-1H-pyrazole-3-sulfonamide;
N′-(3-(4-(1-(2-cyano-1-cyclopentylethyl)-1H-pyrazol-4-yl)pyrimidin-2-ylamino)phenyl)-
N,N-dimethylsulfamide;
N-(4-(4-(1-(2-cyano-1-cyclopentylethyl)-1H-pyrazol-4-yl)pyrimidin-2-ylamino)phenyl)-
2-(pyrrolidin-1-yl)acetamide;
N-(4-(4-(1-(2-cyano-1-cyclopentylethyl)-1H-pyrazol-4-yl)pyrimidin-2-ylamino)phenyl)-
2-(3-hydroxypyrrolidin-1-yl)acetamide;
N-(4-(4-(1-(2-cyano-1-cyclopentylethyl)-1H-pyrazol-4-yl)pyrimidin-2-ylamino)phenyl)-
2-(4-hydroxypiperidin-1-yl)acetamide;
N-(4-(4-(1-(2-cyano-1-cyclopentylethyl)-1H-pyrazol-4-yl)pyrimidin-2-ylamino)phenyl)-
2-(3-oxopiperazin-1-yl)acetamide;
N-(4-(4-(1-(2-cyano-1-cyclopentylethyl)-1H-pyrazol-4-yl)pyrimidin-2-ylamino)phenyl)-
2-morpholinoacetamide;
N-(4-(4-(1-(2-cyano-1-cyclopentylethyl)-1H-pyrazol-4-yl)pyrimidin-2-ylamino)phenyl)-
2-((tetrahydro-2H-pyran-4-yl)methylamino)acetamide;
N-(4-(4-(1-(2-cyano-1-cyclopentylethyl)-1H-pyrazol-4-yl)pyrimidin-2-ylamino)phenyl)-
2-(2-(methoxymethyl)pyrrolidin-1-yl)acetamide;
N-(4-(4-(1-(2-cyano-1-cyclopentylethyl)-1H-pyrazol-4-yl)pyrimidin-2-ylamino)phenyl)-
2-(cyclopropylmethylamino)acetamide;
N-(4-(4-(1-(2-cyano-1-cyclopentylethyl)-1H-pyrazol-4-yl)pyrimidin-2-ylamino)phenyl)-
2-(1-methoxypropan-2-ylamino)acetamide;
2-(4-(5-methylisoxazol-3-yloxy)-1-(4-(2-(4-morpholinophenylamino)pyrimidin-4-yl)-
1H-pyrazol-1-yl)cyclohexyl)acetonitrile;
3-cyclopentyl-3-(4-(2-(4-(morpholine-4-carbonyl)phenylamino)pyrimidin-4-yl)-1H-
pyrazol-1-yl)propanenitrile;
4-(4-(1-(2-cyano-1-cyclopentylethyl)-1H-pyrazol-4-yl)pyrimidin-2-ylamino)-N-
((tetrahydro-2H-1-pyran-4-yl)methyl)benzamide;
3-cyclopentyl-3-(4-(2-(4-((3-endo)-3-hydroxy-8-azabicyclo[3.2.1]octane-8-
carbonyl)phenylamino)pyrimidin-4-yl)-1H-pyrazol-1-yl)propanenitrile;
3-cyclopentyl-3-(4-(2-(4-(2-oxa-6-azatricyclo[3.3.1.1(3,7)]dec-6-
ylcarbonyl)phenyl)aminopyrimidin-4-yl)-1H-pyrazol-1-yl)propanenitrile;
4-(4-(1-(2-cyano-1-cyclopentylethyl)-1H-pyrazol-4-yl)pyrimidin-2-ylamino)-N-(cis-4-
hydroxycyclohexyl)-N-methylbenzamide;
4-(4-(1-(2-cyano-1-cyclopentylethyl)-1H-pyrazol-4-yl)pyrimidin-2-ylamino)-N-methyl-
N-(tetrahydro-2H-pyran-4-yl)benzamide;
3-cyclopentyl-3-(4-(2-(4-(S*)-(4,4-dimethyl-2-oxo-1-oxa-3,7-diazaspiro[4.4]nonane-7-
carbonyl)phenylamino)pyrimidin-4-yl)-1H-pyrazol-1-yl)propanenitrile;
3-cyclopentyl-3-(4-(2-(4-(4,4-dimethyl-1-oxa-7-azaspiro[4.4]nonane-7-
carbonyl)phenylamino)pyrimidin-4-yl)-1H-pyrazol-1-yl)propanenitrile;
3-cyclopentyl-3-(4-(2-(4-(4-methoxypiperidine-1-carbonyl)phenylamino)pyrimidin-4-
yl)-1H-pyrazol-1-yl)propanenitrile;
N-((3S)-1-(4-(4-(1-(2-cyano-1-cyclopentylethyl)-1H-pyrazol-4-yl)pyrimidin-2-
ylamino)benzoyl)pyrrolidin-3-yl)acetamide;
4-(4-(1-(2-cyano-1-cyclopentylethyl)-1H-pyrazol-4-yl)pyrimidin-2-ylamino)-N-(cis-4-
hydroxycyclohexyl)benzamide;
3-(4-(2-(4-(4-acetylpiperazine-1-carbonyl)phenylamino)pyrimidin-4-yl)-1H-pyrazol-1-
yl)-3-cyclopentylpropanenitrile;
(3S)-1-(4-(4-(1-(2-cyano-1-cyclopentylethyl)-1H-1-pyrazol-4-yl)pyrimidin-2-
ylamino)benzoyl)pyrrolidine-3-carbonitrile;
3-cyclopentyl-3-(4-(2-(4-(3-methoxypyrrolidine-1-carbonyl)phenylamino)pyrimidin-4-
yl)-1H-pyrazol-1-yl)propanenitrile;
4-(4-(1-(2-cyano-1-cyclopentylethyl)-1H-pyrazol-4-yl)pyrimidin-2-ylamino)-N-(1-
methylpiperidin-4-yl)benzamide;
3-cyclopentyl-3-(4-(2-(4-(3-oxo-2,8-diazaspiro[4.5]decane-8-
carbonyl)phenylamino)pyrimidin-4-yl)-1H-pyrazol-1-yl)propanenitrile;
3-cyclopentyl-3-(4-(2-(4-(3-fluoropyrrolidine-1-carbonyl)phenylamino)pyrimidin-4-yl)-
1H-pyrazol-1-yl)propanenitrile;
3-cyclopentyl-3-(4-(2-(4-(3-(dimethylamino)pyrrolidine-1-
carbonyl)phenylamino)pyrimidin-4-yl)-1H-pyrazol-1-yl)propanenitrile;
Ethyl 4-(4-(4-(1-(2-cyano-1-cyclopentylethyl)-1H-pyrazol-4-yl)pyrimidin-2-
ylamino)benzamido)piperidine-1-carboxylate;
4-(4-(1-(2-cyano-1-cyclopentylethyl)-1H-pyrazol-4-yl)pyrimidin-2-ylamino)-N-(1-
(pyridin-2-yl)pyrrolidin-3-yl)benzamide;
3-cyclopentyl-3-(4-(2-(4-(3-(pyridin-2-yloxy)pyrrolidine-1-
carbonyl)phenylamino)pyrimidin-4-yl)-1H-pyrazol-1-yl)propanenitrile;
1-(4-(4-(1-(2-cyano-1-cyclopentylethyl)-1H-pyrazol-4-yl)pyrimidin-2-ylamino)benzoyl)-
N,N-dimethylpiperidine-4-carboxamide;
4-(4-(1-(2-cyano-1-cyclopentylethyl)-1H-pyrazol-4-yl)pyrimidin-2-ylamino)-N-(1-
(dimethylamino)-1-oxobutan-2-yl)benzamide;
N-(3-(4-(1-(2-cyano-1-cyclopentylethyl)-1H-pyrazol-4-yl)pyrimidin-2-ylamino)phenyl)-
2-(4-methylpiperazin-1-yl)acetamide;
N-(3-(4-(1-(2-cyano-1-cyclopentylethyl)-1H-pyrazol-4-yl)pyrimidin-2-ylamino)phenyl)-
2-(3-hydroxypyrrolidin-1-yl)acetamide;
N-(3-(4-(1-(2-cyano-1-cyclopentylethyl)-1H-pyrazol-4-yl)pyrimidin-2-ylamino)phenyl)-
2-(3-oxopiperazin-1-yl)acetamide;
N-(3-(4-(1-(2-cyano-1-cyclopentylethyl)-1H-pyrazol-4-yl)pyrimidin-2-ylamino)phenyl)-
2-(4-hydroxypiperidin-1-yl)acetamide;
N-(3-(4-(1-(2-cyano-1-cyclopentylethyl)-1H-pyrazol-4-yl)pyrimidin-2-ylamino)phenyl)-
2-(4-(2-hydroxyethyl)piperazin-1-yl)acetamide;
N-(3-(4-(1-(2-cyano-1-cyclopentylethyl)-1H-pyrazol-4-yl)pyrimidin-2-ylamino)phenyl)-
2-(cyclopropylmethylamino)acetamide;
N-(3-(4-(1-(2-cyano-1-cyclopentylethyl)-1H-pyrazol-4-yl)pyrimidin-2-ylamino)phenyl)-
2-morpholinoacetamide;
N-(3-(4-(1-(2-cyano-1-cyclopentylethyl)-1H-pyrazol-4-yl)pyrimidin-2-ylamino)phenyl)-
2-(ethylamino)acetamide;
2-(4-(5-methylisoxazol-3-yloxy)-1-(4-(2-(4-(3-oxomorpholino)phenylamino)pyrimidin-
4-yl)-1H-pyrazol-1-yl)cyclohexyl)acetonitrile;
2-(4-(5-methylisoxazol-3-yloxy)-1-(4-(2-(4-(2-oxopiperidin-1-
yl)phenylamino)pyrimidin-4-yl)-1H-pyrazol-1-yl)cyclohexyl)acetonitrile;
2-(1-(4-(2-(4-(1H-pyrazol-1-yl)phenylamino)pyrimidin-4-yl)-1H-pyrazol-1-yl)-4-(5-
methylisoxazol-3-yloxy)cyclohexyl)acetonitrile;
2-(4-(5-methylisoxazol-3-yloxy)-1-(4-(2-(3-(oxazol-5-yl)phenylamino)pyrimidin-4-yl)-
1H-pyrazol-1-yl)cyclohexyl)acetonitrile;
3-(cyanomethyl)-3-(4-(2-(4-morpholinophenylamino)pyrimidin-4-yl)-1H-pyrazol-1-
yl)cyclobutanecarbonitrile;
4-(4-(1-(3-(cyanomethyl)-1-(ethylsulfonyl)azetidin-3-yl)-1H-pyrazol-4-yl)pyrimidin-2-
ylamino)benzoic acid;
4-(4-(1-(2-cyano-1-cyclopropylethyl)-1H-pyrazol-4-yl)pyrimidin-2-ylamino)benzoic
acid;
3-cyclopropyl-3-(4-(2-(4-(4-hydroxypiperidine-1-carbonyl)phenylamino)pyrimidin-4-
yl)-1H-pyrazol-1-yl)propanenitrile;
3-cyclopropyl-3-(4-(2-(4-((3-endo)-3-hydroxy-8-azabicyclo[3.2.1]octane-8-
carbonyl)phenylamino)pyrimidin-4-yl)-1H-pyrazol-1-yl)propanenitrile;
3-cyclopropyl-3-(4-(2-(4-(pyrrolidine-1-carbonyl)phenylamino)pyrimidin-4-yl)-1H-
pyrazol-1-yl)propanenitrile;
4-(4-(1-(2-cyano-1-cyclopropylethyl)-1H-pyrazol-4-yl)pyrimidin-2-ylamino)-N-
(tetrahydro-2H-pyran-4-yl)benzamide;
2-(1-(ethylsulfonyl)-3-(4-(2-(4-(morpholine-4-carbonyl)phenylamino)pyrimidin-4-yl)-
1H-pyrazol-1-yl)azetidin-3-yl)acetonitrile;
2-(1-(ethylsulfonyl)-3-(4-(2-(4-(4-hydroxypiperidine-1-
carbonyl)phenylamino)pyrimidin-4-yl)-1H-pyrazol-1-yl)azetidin-3-yl)acetonitrile;
2-(1-(ethylsulfonyl)-3-(4-(2-(4-((3-endo)-3-hydroxy-8-azabicyclo[3.2.1]octane-8-
carbonyl)phenylamino)pyrimidin-4-yl)-1H-pyrazol-1-yl)azetidin-3-yl)acetonitrile;
2-(1-(ethylsulfonyl)-3-(4-(2-(4-(pyrrolidine-1-carbonyl)phenylamino-)pyrimidin-4-yl)-
1H-pyrazol-1-yl)azetidin-3-yl)acetonitrile;
4-(4-(1-(3-(cyanomethyl)-1-(ethylsulfonyl)azetidin-3-yl)-1H-pyrazol-4-yl)pyrimidin-2-
ylamino)-N-(tetrahydro-2H-pyran-4-yl)benzamide;
3-cyclopropyl-3-(4-(2-(4-(morpholine-4-carbonyl)phenylamino)pyrimidin-4-yl)-1H-
pyrazol-1-yl)propanenitrile;
3-(4-(2-(4-(azetidine-1-carbonyl)phenylamino)pyrimidin-4-yl)-1H-pyrazol-1-yl)-3-
cyclopropylpropanenitrile;
3-cyclopropyl-3-(4-(2-(4-(2-oxa-6-azatricyclo[3.3.1.1(3,7)]dec-6-
ylcarbonyl)phenyl)aminopyrimidin-4-yl)-1H-pyrazol-1-yl)propanenitrile;
3-cyclopropyl-3-(4-(2-(4-(4-methoxypiperidine-1-carbonyl)phenylamino)pyrimidin-4-
yl)-1H-pyrazol-1-yl)propanenitrile;
(3R)-1-(4-(4-(1-(2-cyano-1-cyclopropylethyl)-1H-pyrazol-4-yl)pyrimidin-2-
ylamino)benzoyl)pyrrolidine-3-carbonitrile;
3-cyclopropyl-3-(4-(2-(4-(3-methoxypyrrolidine-1-carbonyl)phenylamino)pyrimidin-4-
yl)-1H-pyrazol-1-yl)propanenitrile;
3-cyclopropyl-3-(4-(2-(4-(3-hydroxypyrrolidine-1-carbonyl)phenylamino)pyrimidin-4-
yl)-1H-pyrazol-1-yl)propanenitrile;
3-cyclopropyl-3-(4-(2-(4-(4-methylpiperazine-1-carbonyl)phenylamino)pyrimidin-4-yl)-
1H-pyrazol-1-yl)propanenitrile;
N-((3R)-1-(4-(4-(1-(2-cyano-1-cyclopropylethyl)-1H-pyrazol-4-yl)pyrimidin-2-
ylamino)benzoyl)pyrrolidin-3-yl)acetamide;
3-(4-(2-(4-(4-acetylpiperazine-1-carbonyl)phenylamino)pyrimidin-4-yl)-1H-pyrazol-1-
yl)-3-cyclopropylpropanenitrile;
3-cyclopropyl-3-(4-(2-(4-(3-(dimethylamino)pyrrolidine-1-
carbonyl)phenylamino)pyrimidin-4-yl)-1H-pyrazol-1-yl)propanenitrile;
3-cyclopropyl-3-(4-(2-(4-(3-fluoropyrrolidine-1-carbonyl)phenylamino)pyrimidin-4-yl)-
1H-pyrazol-1-yl)propanenitrile;
Ethyl 4-(4-(4-(1-(2-cyano-1-cyclopropylethyl)-1H-pyrazol-4-yl)pyrimidin-2-
ylamino)benzoyl)aminopiperidine-1-carboxylate;
2-(3-(4-(2-(4-(azetidine-1-carbonyl)phenylamino)pyrimidin-4-yl)-1H-pyrazol-1-yl)-1-
(ethylsulfonyl)azetidin-3-yl)acetonitrile;
1-(ethylsulfonyl)-3-(4-(2-(4-(2-oxa-6-azatricyclo[3.3.1.1(3,7)]dec-6-
ylcarbonyl)phenyl)aminopyrimidin-4-yl)-1H-pyrazol-1-yl)azetidin-3-ylacetonitrile;
(1-(ethylsulfonyl)-3-4-(2-(4-((4-methoxypiperidin-1-
yl)carbonyl)phenylamino)pyrimidin-4-yl)-1H-pyrazol-1-ylazetidin-3-yl)acetonitrile;
1-(4-(4-(1-(3-(cyanomethyl)-1-(ethylsulfonyl)azetidin-3-yl)-1H-pyrazol-4-yl)pyrimidin-
2-ylamino)benzoyl)pyrrolidine-3-carbonitrile;
2-(1-(ethylsulfonyl)-3-(4-(2-(4-(3-methoxypyrrolidine-1-
carbonyl)phenylamino)pyrimidin-4-yl)-1H-pyrazol-1-yl)azetidin-3-yl)acetonitrile;
2-(1-(ethylsulfonyl)-3-(4-(2-(4-(3-hydroxypyrrolidine-1-
carbonyl)phenylamino)pyrimidin-4-yl)-1H-pyrazol-1-yl)azetidin-3-yl)acetonitrile;
2-(1-(ethylsulfonyl)-3-(4-(2-(4-(4-methylpiperazine-1-carbonyl)phenylamino)pyrimidin-
4-yl)-1H-pyrazol-1-yl)azetidin-3-yl)acetonitrile;
N-(1-(4-(4-(1-(3-(cyanomethyl)-1-(ethylsulfonyl)azetidin-3-yl)-1H-pyrazol-4-
yl)pyrimidin-2-ylamino)benzoyl)pyrrolidin-3-yl)acetamide;
2-(3-(4-(2-(4-(4-acetylpiperazine-1-carbonyl)phenylamino)pyrimidin-4-yl)-1H-pyrazol-
1-yl)-1-(ethylsulfonyl)azetidin-3-yl)acetonitrile;
2-(3-(4-(2-(4-(3-(dimethylamino)pyrrolidine-1-carbonyl)phenylamino)pyrimidin-4-yl)-
1H-pyrazol-1-yl)-1-(ethylsulfonyl)azetidin-3-yl)acetonitrile;
2-(1-(ethylsulfonyl)-3-(4-(2-(4-(3-fluoropyrrolidine-1-carbonyl)phenylamino)pyrimidin-
4-yl)-1H-pyrazol-1-yl)azetidin-3-yl)acetonitrile;
Ethyl 4-(4-(4-(1-(3-(cyanomethyl)-1-(ethylsulfonyl)azetidin-3-yl)-1H-pyrazol-4-
yl)pyrimidin-2-ylamino)benzamido)piperidine-1-carboxylate;
4-(4-(1-(3-cyano-1-(cyanomethyl)cyclobutyl)-1H-pyrazol-4-yl)pyrimidin-2-
ylamino)benzoic acid;
4-(4-(1-(2-cyano-1-cyclopentylethyl)-1H-pyrazol-4-yl)-5-methoxypyrimidin-2-
ylamino)benzoic acid;
3-(cyanomethyl)-3-(4-(2-(4-(morpholine-4-carbonyl)phenylamino)pyrimidin-4-yl)-1H-
pyrazol-1-yl)cyclobutanecarbonitrile;
3-(cyanomethyl)-3-(4-(2-(4-(pyrrolidine-1-carbonyl)phenylamino)pyrimidin-4-yl)-1H-
pyrazol-1-yl)cyclobutanecarbonitrile;
4-(4-(1-(3-cyano-1-(cyanomethyl)cyclobutyl)-1H-pyrazol-4-yl)pyrimidin-2-ylamino)-N-
(tetrahydro-2H-pyran-4-yl)benzamide;
3-(cyanomethyl)-3-(4-(2-(4-(3-hydroxypyrrolidine-1-carbonyl)phenylamino)pyrimidin-
4-yl)-1H-pyrazol-1-yl)cyclobutanecarbonitrile;
4-(4-(1-(3-cyano-1-(cyanomethyl)cyclobutyl)-1H-pyrazol-4-yl)pyrimidin-2-ylamino)-N-
((5-methylisoxazol-3-yl)methyl)benzamide;
3-(4-(2-(4-(azetidine-1-carbonyl)phenylamino)pyrimidin-4-yl)-1H-pyrazol-1-yl)-3-
(cyanomethyl)cyclobutanecarbonitrile;
3-(cyanomethyl)-3-(4-(2-(4-(4-methylpiperazine-1-carbonyl)phenylamino)pyrimidin-4-
yl)-1H-pyrazol-1-yl)cyclobutanecarbonitrile;
3-(cyanomethyl)-3-(4-(2-(4-(3-fluoropyrrolidine-1-carbonyl)phenylamino)pyrimidin-4-
yl)-1H-pyrazol-1-yl)cyclobutanecarbonitrile;
3-(cyanomethyl)-3-(4-(2-(4-(4-methoxypiperidine-1-carbonyl)phenylamino)pyrimidin-4-
yl)-1H-pyrazol-1-yl)cyclobutanecarbonitrile;
3-(cyanomethyl)-3-(4-(2-(4-(3-methoxypyrrolidine-1-carbonyl)phenylamino)pyrimidin-
4-yl)-1H-pyrazol-1-yl)cyclobutanecarbonitrile;
1-(4-(4-(1-(3-cyano-1-(cyanomethyl)cyclobutyl)-1H-pyrazol-4-yl)pyrimidin-2-
ylamino)benzoyl)pyrrolidine-3-carbonitrile;
3-(4-(2-(4-(4-acetylpiperazine-1-carbonyl)phenylamino)pyrimidin-4-yl)-1H-pyrazol-1-
yl)-3-(cyanomethyl)cyclobutanecarbonitrile;
N-(1-(4-(4-(1-(3-cyano-1-(cyanomethyl)cyclobutyl)-1H-pyrazol-4-yl)pyrimidin-2-
ylamino)benzoyl)pyrrolidin-3-yl)acetamide;
3-(cyanomethyl)-3-(4-(2-(4-((3-endo)-3-hydroxy-8-azabicyclo[3.2.1]octane-8-
carbonyl)phenylamino)pyrimidin-4-yl)-1H-pyrazol-1-yl)cyclobutanecarbonitrile;
3-(cyanomethyl)-3-(4-(2-(4-(4-hydroxypiperidine-1-carbonyl)phenylamino)pyrimidin-4-
yl)-1H-pyrazol-1-yl)cyclobutanecarbonitrile;
3-cyclopentyl-3-(4-(5-methoxy-2-(4-(morpholine-4-carbonyl)phenylamino)pyrimidin-4-
yl)-1H-pyrazol-1-yl)propanenitrile;
3-cyclopentyl-3-(4-(5-methoxy-2-(4-(pyrrolidine-1-carbonyl)phenylamino)pyrimidin-4-
yl)-1H-pyrazol-1-yl)propanenitrile;
3-cyclopentyl-3-(4-(5-methoxy-2-(4-(4-methylpiperazine-1-
carbonyl)phenylamino)pyrimidin-4-yl)-1H-pyrazol-1-yl)propanenitrile;
4-(4-(1-(2-cyano-1-cyclopentylethyl)-1H-pyrazol-4-yl)-5-methoxypyrimidin-2-ylamino)-
N-(tetrahydro-2H-pyran-4-yl)benzamide
3-(4-(7H-pyrrolo[2,3-d]pyrimidin-4-yl)-1H-pyrazol-1-yl)-3-(3-
hydroxycyclopentyl)propanenitrile;
3-(4-(7H-pyrrolo[2,3-d]pyrimidin-4-yl)-1H-pyrazol-1-yl)-3-(2-
hydroxycyclopentyl)propanenitrile;
3-(4-(7H-pyrrolo[2,3-d]pyrimidin-4-yl)-1H-pyrazol-1-yl)-3-(3-
oxocyclopentyl)propanenitrile
(R)-3-(4-(7H-pyrrolo[2,3-d]pyrimidin-4-yl)-1H-pyrazol-1-yl)-3-cyclo-
pentylpropanenitrile maleic acid salt;
(R)-3-(4-(7H-pyrrolo[2,3-d]pyrimidin-4-yl)-1H-pyrazol-1-yl)-3-
cyclopentylpropanenitrile sulfuric acid salt;
(R)-3-(4-(7H-pyrrolo[2,3-d]pyrimidin-4-yl)-1H-pyrazol-1-yl)-3-
cyclopentylpropanenitrile phosphoric acid salt;
2-(3-(4-(7H-pyrrolo[2,3-d]pyrimidin-4-yl)-1H-pyrazol-1-yl)-1-
(ethylsulfonyl)azetidin-3-yl)acetonitrile(baricitinib);
N-(4-((4-(3-hydroxy-3-isopropylazetidin-1-yl)-6-((3-methyl-1H-pyrazol-5-
yl)amino)pyrimidin-2-yl)thio)phenyl)propionamide;
N-(4-((4-((5-cyclopropyl-1H-pyrazol-3-yl)amino)-6-(3-cyclopropyl-3-hydroxyazetidin-1-
yl)pyrimidin-2-yl)thio)phenyl)propionamide;
N-(4-((4-((5-cyclopropyl-1H-pyrazol-3-yl)amino)-6-(3-cyclopropyl-3-hydroxyazetidin-1-
yl)pyrimidin-2-yl)thio)phenyl)cyclopropanecarboxamide;
N-(4-((4-(3-cyclopropyl-3-hydroxyazetidin-1-yl)-6-((3-methyl-1H-pyrazol-5-
yl)amino)pyrimidin-2-yl)thio)phenyl)-2,2-difluorocyclopropanecarboxamide;
N-(4-((4-(3-cyclopropyl-3-fluoroazetidin-1-yl)-6-((3-methyl-1H-pyrazol-5-
yl)amino)pyrimidin-2-yl)thio)phenyl)-3,3,3-trifluoropropanamide;
1-(4-(4-(1H-pyrrolo[2,3-b]pyridin-3-yl)pyrimidin-2-yl)-1,4-diazepan-1-yl)ethanone;
N-benzyl-4-(1H-pyrrolo[2,3-b]pyridin-3-yl)pyrimidin-2-amine;
3-(2-(1,4-diazepan-1-yl)pyrimidin-4-yl)-1H-pyrrolo[2,3-b]pyridine;
(R)-1-(4-(4-(1H-pyrrolo[2,3-b]pyridin-3-yl)pyrimidin-2-yl)-2-methylpiperazin-1-
yl)ethanone;
1-((2S,6R)-4-(4-(1H-pyrrolo[2,3-b]pyridin-3-yl)pyrimidin-2-yl)-2,6-dimethylpiperazin-
1-yl)ethanone;
N-(cyclohexylmethyl)-4-(1H-pyrrolo[2,3-b]pyridin-3-yl)pyrimidin-2-amine;
(S)-1-(4-(4-(1H-pyrrolo[2,3-b]pyridin-3-yl)pyrimidin-2-yl)-2-methylpiperazin-1-
yl)ethanone;
2-(5-chloro-1H-pyrrolo[2,3-b]pyridin-3-yl)-5-fluoro-N-(1-methylpyrrolidin-3-
yl)pyrimidin-4-amine;
N-benzyl-2-(1H-pyrrolo[2,3-b]pyridin-3-yl)pyrimidin-4-amine;
(S)-2-(5-chloro-1H-pyrrolo[2,3-b]pyridin-3-yl)-5-fluoro-N-(1-methylpyrrolidin-3-
yl)pyrimidin-4-amine;
2-(5-chloro-1H-pyrrolo[2,3-b]pyridin-3-yl)-N-cyclohexyl-5-fluoropyrimidin-4-amine;
2-(5-chloro-1H-pyrrolo[2,3-b]pyridin-3-yl)-5-fluoro-N-(pyrrolidin-3-yl)pyrimidin-4-
amine;
2-(5-chloro-1H-pyrrolo[2,3-b]pyridin-3-yl)-5-fluoro-N-(o-tolyl)pyrimidin-4-amine;
2-(5-chloro-1H-pyrrolo[2,3-b]pyridin-3-yl)-5-fluoro-N-(piperidin-3-yl)pyrimidin-4-
amine;
2-(5-chloro-1H-pyrrolo[2,3-b]pyridin-3-yl)-5-fluoro-N-(tetrahydro-2H-pyran-4-
yl)pyrimidin-4-amine;
2-((2-(5-chloro-1H-pyrrolo[2,3-b]pyridin-3-yl)-5-fluoropyrimidin-4-yl)amino)-2,3-
dimethyl-N-(2,2,2-trifluoroethyl)butanamide;
2-(5-chloro-1H-pyrrolo[2,3-b]pyridin-3-yl)-N-(2,6-dimethylphenyl)-5-fluoropyrimidin-
4-amine;
2-((2-(5-chloro-1H-pyrrolo[2,3-b]pyridin-3-yl)-5-fluoropyrimidin-4-yl)amino)-2-methyl-
N-(2,2,2-trifluoroethyl)propanamide;
2-((2-(5-chloro-1H-pyrrolo[2,3-b]pyridin-3-yl)-5-fluoropyrimidin-4-yl)amino)-2-methyl-
N-(2,2,2-trifluoroethyl)butanamide;
2-((2-(5-chloro-1H-pyrrolo[2,3-b]pyridin-3-yl)-5-fluoropyrimidin-4-yl)amino)-2,3-
dimethyl-N-(2,2,2-trifluoroethyl)butanamide;
N-(2,2-difluoroethyl)-2-((5-fluoro-2-(5-fluoro-1H-pyrrolo[2,3-b]pyridin-3-yl)pyrimidin-
4-yl)amino)-2-methylpropanamide;
2-((2-(5-chloro-1H-pyrrolo[2,3-b]pyridin-3-yl)-5-fluoropyrimidin-4-yl)amino)-2-ethyl-
N-(2,2,2-trifluoroethyl)butanamide;
2-((5-fluoro-2-(5-fluoro-1H-pyrrolo[2,3-b]pyridin-3-yl)pyrimidin-4-yl)amino)-N-(2-
fluoroethyl)-2-methylpropanamide;
2-((5-fluoro-2-(5-fluoro-1H-pyrrolo[2,3-b]pyridin-3-yl)pyrimidin-4-yl)amino)-2-methyl-
N-(2,2,2-trifluoroethyl)butanamide;
2-((2-(5-chloro-1H-pyrrolo[2,3-b]pyridin-3-yl)-5-fluoropyrimidin-4-yl)amino)-N-(2,2-
difluoroethyl)-2-methylpropanamide;
2-((2-(5-chloro-1H-pyrrolo[2,3-b]pyridin-3-yl)-5-fluoropyrimidin-4-yl)amino)-N-(2-
fluoroethyl)-2-methylpropanamide;
2-(5-chloro-1H-pyrrolo[2,3-b]pyridin-3-yl)-5-fluoro-N-(1,2,3,4-tetrahydronaphthalen-1-
yl)pyrimidin-4-amine;
2-((5-fluoro-2-(5-fluoro-1H-pyrrolo[2,3-b]pyridin-3-yl)pyrimidin-4-yl)amino)-2-methyl-
N-(2,2,2-trifluoroethyl)propanamide;
4-((2-(5-chloro-1H-pyrrolo[2,3-b]pyridin-3-yl)-5-fluoropyrimidin-4-
yl)amino)benzamide;
1-((2S,6R)-4-(4-(5-chloro-1H-pyrrolo[2,3-b]pyridin-3-yl)pyrimidin-2-yl)-2,6-
dimethylpiperazin-1-yl)ethanone;
2-(5-chloro-1H-pyrrolo[2,3-b]pyridin-3-yl)-N-(2,3-dihydro-1H-inden-1-yl)-5-
fluoropyrimidin-4-amine;
N-benzyl-4-(5-chloro-1H-pyrrolo[2,3-b]pyridin-3-yl)pyrimidin-2-amine;
1-((2R,6S)-4-(4-(5-methoxy-1H-pyrrolo[2,3-b]pyridin-3-yl)pyrimidin-2-yl)-2,6-
dimethylpiperazin-1-yl)ethanone;
5-methoxy-3-(2-(4-(methylsulfonyl)-1,4-diazepan-1-yl)pyrimidin-4-yl)-1H-pyrrolo[2,3-
b]pyridine;
(S)-2-((4-(5-chloro-1H-pyrrolo[2,3-b]pyridin-3-yl)pyrimidin-2-yl)amino)-4-
methylpentan-1-ol;
(S)-2-((4-(5-chloro-1H-pyrrolo[2,3-b]pyridin-3-yl)pyrimidin-2-yl)amino)-4-
methylpentan-1-ol;
N-(4-(1H-imidazol-1-yl)phenyl)-2-(5-chloro-1H-pyrrolo[2,3-b]pyridin-3-yl)-5-
fluoropyrimidin-4-amine;
ethyl 4-(4-(5-methoxy-1H-pyrrolo[2,3-b]pyridin-3-yl)pyrimidin-2-yl)-1,4-diazepane-1-
carboxylate;
2-(5-chloro-1H-pyrrolo[2,3-b]pyridin-3-yl)-5-fluoro-N-(piperidin-4-yl)pyrimidin-4-
amine;
N-(cyanomethyl)-4-(2-((4-morpholinophenyl)amino)pyrimidin-4-yl)benzamide;
N-(tert-butyl)-3-((5-methyl-2-((4-(2-(pyrrolidin-1-yl)ethoxy)phenyl)amino)pyrimidin-4-
yl)amino)benzenesulfonamide;
(5S,6S,8R)-6-hydroxy-6-(hydroxymethyl)-5-methyl-7,8,14,15-tetrahydro-5H-16-oxa-
4b,8a,14-triaza-5,8-methanodibenzo[b,h]cycloocta[jkl]cyclopenta[e]-as-indacen-13(6H)-one
or combinations thereof.
*Particular compounds provided herein were named using the ChemDraw Ultra 12.0 program.

In certain disclosed embodiments, the one or more compounds are pyridine-containing compounds. An example of a such pyridine-containing compound is cyclopropanecarboxylic acid {5-[4-(1,1-dioxo-1-thiomorpholin-4ylmethyl)-phenyl]-[1,2,4]triazaolo[1,5-a]pyridine-2-yl}-amide.

In particular disclosed embodiments, the compound is selected from the following: 3-{(3R,4R)-4-methyl-3-[methyl-(7H-pyrrolo[2,3-d]pyrimidin-4-yl)-amino]-piperidin-1-yl}-3-oxo-propionitrile (tofacitinib) and the citrate salt thereof

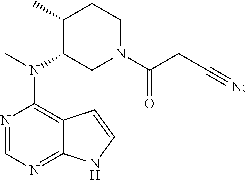 [SMILES: C[C@@H]1CCN(C(=O)CC#N)C[C@@H]1N(C)c1ncnc2[nH]ccc12]

(R)-3-(4-(7H-pyrrolo[2,3-d]pyrimidin-4-yl)-1H-pyrazol-1-yl)-3-cyclopentyl-propanenitrile (ruxolitinib)

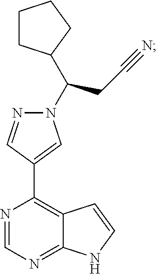

2-(3-(4-(7H-pyrrolo[2,3-d]pyrimidin-4-yl)-1H-pyrazol-1-yl)-1-(ethylsulfonyl)azetidin-3-yl)acetonitrile (baricitinib)

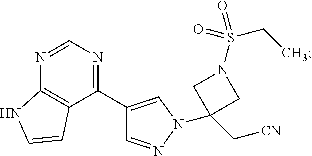 [SMILES: CCS(=O)(=O)N1CC(CC#N)(n2cc(-c3ncnc4[nH]ccc34)cn2)C1]

N-(tert-butyl)-3-methyl-2-((4-(2-(pyrrolidin-1-yl)ethoxy)phenyl)amino)pyrimidin-4-yl)amino)benzenesulfonamide

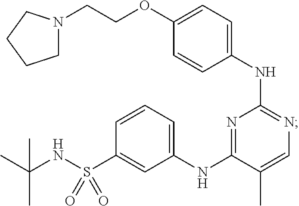 [SMILES: Cc1cnc(Nc2ccc(OCCN3CCCC3)cc2)nc1Nc1cccc(S(=O)(=O)NC(C)(C)C)c1]

(5S,6S,8R)-6-hydroxy-6-(hydroxymethyl)-5-methyl-7, 8,14,15-tetrahydro-5H-16-oxa-4b,8a,14-triaza-5,8-methanodibenzo[b,h]cycloocta[jkl]cyclopenta[e]-as-indacen-13(6H)-one (lestaurtinib)

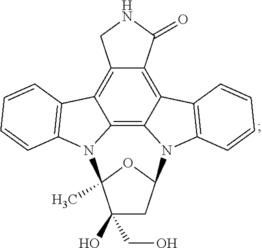 [SMILES: C[C@]12O[C@H](C[C@]1(O)CO)n1c3ccccc3c3c4c(c5c6ccccc6n2c5c31)CNC4=O]

and
N-(cyanomethyl)-4-(2-((4-morpholinophenyl)amino)pyrimidin-4-yl)benzamide

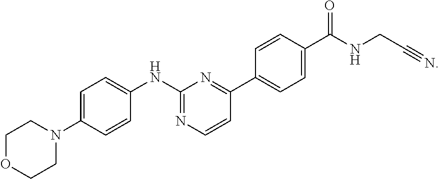 [SMILES: N#CCNC(=O)c1ccc(-c2ccnc(Nc3ccc(N4CCOCC4)cc3)n2)cc1]

As is recognized by one of ordinary skill in the art, the present formulae include other salt forms in addition to those specifically described herein. Similarly, one of ordinary skill in the art would understand the presently disclosed formulae to encompass solvates, such as hydrates.

B. Prodrugs

Those of ordinary skill in the art will appreciate that the compounds described herein can include functional groups that can be masked with progroups to create prodrugs. Such prodrugs are usually, but need not be, pharmacologically inactive until converted into their active drug form. Indeed, many of the disclosed compounds described in this invention include progroups that are hydrolyzable or otherwise cleavable under conditions of use.

The mechanism by which the progroup(s) metabolizes is not critical and can be caused, for example, by hydrolysis under the acidic conditions of the stomach, as described above, and/or by enzymes present in the digestive tract and/or tissues or organs of the body. Indeed, the progroup(s) can be selected to metabolize at a particular site within the body. For example, many esters are cleaved under the acidic conditions found in the stomach. Prodrugs designed to cleave chemically in the stomach to the active compound can employ progroups including such esters. Alternatively, the progroups can be designed to metabolize in the presence of enzymes such as esterases, amidases, lipolases, and phosphatases, including ATPases and kinase, etc. Progroups including linkages capable of metabolizing in vivo are well known and include, by way of example and not limitation, ethers, thioethers, silylethers, silylthioethers, esters, thioesters, carbonates, thiocarbonates, carbamates, thiocarbamates, ureas, thioureas, and carboxamides. In some instances, a “precursor” group that is oxidized by oxidative enzymes such as, for example, cytochrome P₄₅₀ of the liver, to a metabolizable group, can be selected.

In the prodrugs, any available functional moiety (or moieties) can be masked with a progroup (or progroups) to yield a prodrug. Functional groups within the compounds that can be masked with progroups for inclusion in a progroup include, but are not limited to, amines (primary and secondary), hydroxyls, sulfanyls (thiols), and carboxyls. A wide variety of progroups, as well as the resultant progroups, suitable for masking functional groups in active compounds to yield prodrugs are well-known in the art. For example, a hydroxyl functional group can be masked as a sulfonate, ester, or carbonate progroup, which can be hydrolyzed in vivo to provide the hydroxyl group. An amino functional group can be masked as an amide, carbamate, imine, urea, phosphenyl, phosphoryl, or sulfenyl progroup, which can be hydrolyzed in vivo to provide the amino group. A carboxyl group can be masked as an ester (including silyl esters and thioesters), amide, or hydrazide progroup, which can be hydrolyzed in vivo to provide the carboxyl group. Other specific examples of suitable progroups and their respective progroups will be apparent to those of ordinary skill in the art. All of these progroups, alone or in combinations, can be included in the prodrugs.

In some embodiments of the disclosed compounds, and methods of using the compounds, the compound is a 2,4-pyrimidinediamine compound and the progroup(s) can be attached to any available primary or secondary amine, including, for example, the N2 nitrogen atom of the 2,4-pyrimidinediamine, the N4 nitrogen atom of the 2,4-pyrimidinediamine, and/or a primary or secondary nitrogen atom included in a substituent on the 2,4-pyrimidinediamine.

The progroup can include virtually any known or later-discovered hydroxyl, amine or thiol protecting group. Non-limiting examples of suitable protecting groups can be found, for example, in Protective Groups in Organic Synthesis, Greene & Wuts, 2^{nd} Ed., John Wiley & Sons, New York, 1991 (especially pages 10-142 (alcohols, 277-308 (thiols) and 309-405 (amines) the disclosure of which is incorporated herein by reference, herein referred to as “Green & Wuts”).

A particularly useful progroup employed in exemplary disclosed compounds is —CH₂OP(OH)₂ as well as esters, mixed acid esters and salts thereof. In some embodiments, the —CH₂OP(OH)₂ progroup is attached via a nitrogen atom, annular or not, of the parent molecule. There can be more than one such progroup. Thus, one embodiment is a compound of formula I,

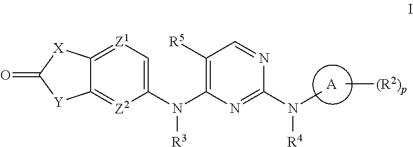

or solvate thereof, where A, X, Y, Z¹, Z², R², R³, R⁴, R⁵ and p are as described herein above, and at least one of R′(when present), R³ and R⁴ is R⁵⁰; where R⁵⁰ is —CH₂OP(O)(OR¹¹)₂; each R¹¹ is independently for each occurrence H, C₁₋₆alkyl or monovalent cationic group, or two R¹¹, together with the atoms to which they are attached, form a 4-8 membered cyclic phosphate group

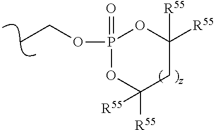

where each R⁵⁵ is independently for each occurrence H, optionally substituted C₁₋₆alkyl, optionally substituted 3-8 membered heteroalicyclyl, optionally substituted C₆₋₁₄aryl, optionally substituted C₇₋₂₀arylalkyl, optionally substituted 5-14 membered heteroaryl or optionally substituted 6-15 membered heteroarylalkyl; z is 0, 1, 2 or 3; or two R¹¹ together represent a divalent organic or inorganic cationic group, wherein exemplary inorganic divalent cationic groups include those selected from Ba²⁺, Bi²⁺, Ca²⁺, Cu²⁺, Mg²⁺, N²⁺, Sr²⁺ and Zn²⁺.

Another embodiment of the presently disclosed compounds includes compounds of formula Ia

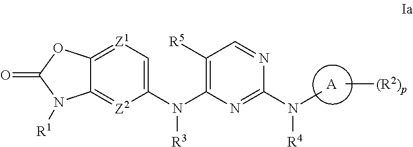

or solvate thereof, where A, Z¹, Z², R², R³, R⁴, R⁵ and p are as described herein above, and at least one of R¹, R³ and R⁴ is R⁵⁰; where R⁵⁰ is —CH₂OP(O)(OR¹¹)₂; each R¹¹ is independently for each occurrence H, C₁₋₆alkyl or a monovalent cationic group, or two R¹¹, together with the atoms to which they are attached, form a 5 or 6-membered cyclic phosphate group, where —CH₂OP(O)(OR¹¹)₂ is

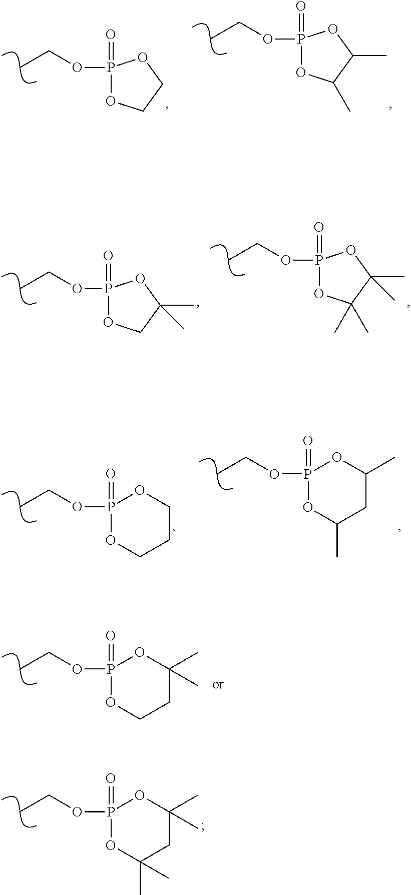

or two R¹¹ together represent a pharmaceutically acceptable divalent cationic group, by way of example including those selected from Ca²⁺, Mg²⁺ and Zn²⁺.

Another embodiment is a compound of formula Ib

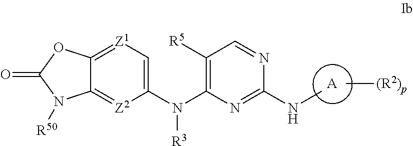

or solvate thereof, where A, Z¹, Z², R², R⁵ and p are as described herein above, and R⁵⁰ is —CH₂OP(O)(OR¹¹)₂; each R¹¹ is independently for each occurrence H, C₁₋₆alkyl, Li⁺, K⁺, HOCH₂CH₂N(CH₃)₃⁺, Na⁺ or NH₄⁺; or two R¹¹ together represent a divalent cationic group selected from Ca²⁺, Mg²⁺ and Zn²⁺.

Another embodiment is a compound of formula IC

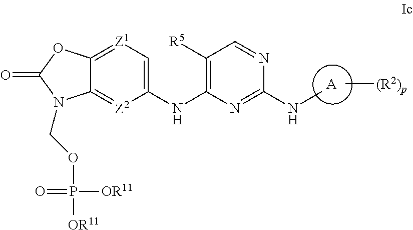

or solvate thereof, where A, Z¹, Z², R², R⁵ and p are as described herein above, and each R¹¹ is independently for each occurrence H, t-butyl, Li⁺, HOCH₂CH₂N(CH₃)₃⁺, K⁺, Na⁺ or NH₄⁺; or two R¹¹ together represent a divalent cationic group selected from Ca²⁺, Mg²⁺ and Zn²⁺.

While not intending to be bound by any particular theory of operation, it is believed that progroups —CH₂OP(O)(OR¹¹)₂, e.g according to formula Ic, metabolize to active compounds via the corresponding hydroxymethylamine intermediate illustrated below:

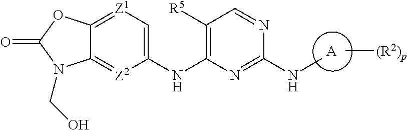

Such hydroxymethylamine compounds, although typically isolable under controlled conditions, are known to be unstable under physiological conditions and various pH ranges where they hydrolyze in vivo to yield formaldehyde and the active drug substance. Based on this observation, compounds of the invention include hydroxymethyl progroups that can be metabolized in vivo, for example by the acidic conditions of the stomach and/or by enzymes present in the digestive tract or other organs and/or tissues or fluids with the body, to yield the active drug substance.

Moreover, it is expected that the amino and thio analogs of these hydroxymethylamines will be similarly unstable at physiological conditions and also hydrolyze in vivo to the active drug. Accordingly, compounds of the invention include these corresponding primary amino and thiol compounds. Also, the invention includes compounds in which the primary amine, thiol and hydroxy groups are masked with “protecting” groups that are removed under physiological conditions to yield the corresponding hydroxymethyl, thiolmethyl and aminomethyl compounds, that is, with these “protecting groups” these compounds will likewise make suitable prodrugs.

Suitability of any particular progroup for a desired mode of administration can be confirmed in biochemical assays. For example, if a prodrug is to be administered by injection into a particular tissue or organ and the identities of the various enzyme(s) expressed in the tissue or organ are known, the particular prodrug can be tested for metabolism in biochemical assays with the isolated enzyme(s). Alternatively, the particular prodrug can be tested for metabolism to the active compound with tissue and/or organ extracts. Using tissue and/or organ extracts can be of particular convenience when the identity(ies) of the enzymes expressed in the target tissues or organs are unknown or in instances when the isolated enzymes are not conveniently available. Persons of ordinary skill in the art will be able to readily select progroups having metabolic properties (such as kinetics) suitable for particular applications using such in vitro tests. Specific prodrugs could also be tested for suitable metabolism in vitro animal models.

Compounds of the invention bearing the —CH₂OP(O)(OR¹¹)₂ progroup can be synthesized, e.g., as depicted below for compounds of formula Ic.

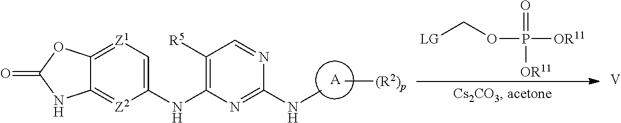

Typically, the proton on the NH of the oxazolidinone ring can be selectively alkylated with the appropriate phosphonate reagent, where LG is a suitable leaving group to form compounds of the invention, in this case of formula Ic. Further description of how to make progroups of formula —CH₂OP(O)(OR¹¹)₂ as described herein is specifically taught in U.S. Pat. No. 7,449,458, entitled “Pyrimidinediamine Prodrugs and their Uses,” the disclosure of which is hereby incorporated by reference in its entirety.

One of ordinary skill in the art will appreciate that compounds of the invention may exhibit the phenomena of tautomerism, conformational isomerism, geometric isomerism, and/or optical isomerism. For example, the compounds and prodrugs of the invention can include one or more chiral centers and/or double bonds and as a consequence can exist as stereoisomers, such as double-bond isomers (such as, geometric isomers), enantiomers, diasteromers, and mixtures thereof, such as racemic mixtures. As another example, the compounds of the invention can exist in several tautomeric forms, including the enol form, the keto form, and mixtures thereof. As the various compound names, formulae and compound drawings within the specification and claims can represent only one of the possible tautomeric, conformational isomeric, optical isomeric, or geometric isomeric forms, it would be understood that the invention encompasses any tautomeric, conformational isomeric, optical isomeric, and/or geometric isomeric forms of the compounds described herein, as well as mixtures of these various different isomeric forms. In cases of limited rotation, e.g. around the core structure of particular embodiments of the disclosed compound, atropisomers are also possible and are also specifically included in the compounds of the invention. It is intended that the compounds encompassed herein are, with the exception of forms of isomerism, chemically stable and isolable.

As is understood by one of ordinary skill in the art, certain atoms occur in more than one isotopic form. For example hydrogen occurs as protium (¹H), deuterium (²H) and tritium (³H), and carbon occurs naturally as three different isotopes, ¹²C, ¹³C and ¹⁴C. Thus the presently disclosed formulas include compounds having one or more different isotopic forms of certain elements, including hydrogen and carbon. In one embodiment of the disclosure, the presently disclosed compounds are provided in isotopically enriched form. In particular examples, compounds of formula I are enriched in deuterium relative to protium.

Deuterium has a natural abundance of about 0.015%. Accordingly, for approximately every 6,500 hydrogen atoms occurring in nature, there is one deuterium atom. Disclosed herein are compounds enriched in deuterium at one or more positions. Thus, deuterium containing compounds of the disclosure have deuterium at one or more positions (as the case may be) in an abundance of greater than 0.015%.

In one embodiment, a compound of formula (I), at a position designated as having deuterium, has a minimum isotopic enrichment factor of at least 2000 (30% deuterium incorporation) at each atom designated as deuterium in the compound, or at least 3000 (45% deuterium incorporation).

In other embodiments, a compound of formula (I) has an isotopic enrichment factor for each designated deuterium atom of at least 3500 (52.5% deuterium incorporation at each designated deuterium atom), at least 4000 (60% deuterium incorporation), at least 4500 (67.5% deuterium incorporation), at least 5000 (75% deuterium incorporation), at least 5500 (82.5% deuterium incorporation), at least 6000 (90% deuterium incorporation), at least 6333.3 (95% deuterium incorporation), at least 6466.7 (97% deuterium incorporation), at least 6600 (99% deuterium incorporation), or at least 6633.3 (99.5% deuterium incorporation).

C. Compositions

Another embodiment is a pharmaceutical composition including a compound as described in any of the embodiments above. Pharmaceutical compositions described herein can be manufactured by means of conventional mixing, dissolving, granulating, dragee-making, levigating, emulsifying, encapsulating, entrapping, lyophilization, and like processes. The compositions can be formulated in conventional manner using one or more physiologically acceptable carriers, diluents, excipients, or auxiliaries which facilitate processing of the active compounds into preparations which can be used pharmaceutically.

The disclosed compound can be formulated in the pharmaceutical compositions per se, or in the form of a hydrate, solvate, N-oxide, or pharmaceutically acceptable salt, as described herein. Typically, such salts are more soluble in aqueous solutions than the corresponding free acids and bases, but salts having lower solubility than the corresponding free acids and bases can also be formed.

One embodiment is a pharmaceutical formulation including a compound of Formulas I-VIII, as described herein, or a prodrug thereof, and at least one pharmaceutically acceptable excipient, diluent, preservative, stabilizer, or mixture thereof.

In one embodiment, the compounds are provided as non-toxic pharmaceutically acceptable salts, as noted previously. Suitable pharmaceutically acceptable salts of the compounds described herein include acid addition salts, such as those formed with suitable inorganic and organic acids (such as hydrochloric acid, fumaric acid, p-toluenesulphonic acid, maleic acid, succinic acid, acetic acid, citric acid, tartaric acid, carbonic acid, or phosphoric acid). Salts of amine groups can also include quaternary ammonium salts in which the amino nitrogen atom carries a suitable organic group such as an alkyl, alkenyl, alkynyl, or substituted alkyl moiety. Furthermore, where presently disclosed compounds carry an acidic moiety, suitable pharmaceutically acceptable salts thereof can include metal salts such as alkali metal salts, e.g., sodium or potassium salts; and alkaline earth metal salts, e.g., calcium or magnesium salts.

The pharmaceutical compositions for the administration of the compounds can be conveniently presented in dosage unit form and can be prepared by any of the methods well known in the art of pharmacy. The pharmaceutical compositions can be, for example, prepared by uniformly and intimately bringing the active ingredient into association with a liquid carrier, a finely divided solid carrier or both, and then, if necessary, shaping the product into the desired formulation. In the pharmaceutical composition the active object compound is included in an amount sufficient to produce the desired therapeutic effect.

D. Second Therapeutic Agents

Disclosed embodiments of the compound may be administered singly, as compositions comprising one or more disclosed embodiments of the compound, or as compositions comprising the compound and a second therapeutic agent.

In particular disclosed embodiments, the second therapeutic agent may be selected from any of the following:
analgesics—morphine, fentanyl, hydromorphone, oxycodone, codeine, acetaminophen, hydrocodone, buprenorphine, tramadol, venlafaxine, flupirtine, meperidine, pentazocine, dextromoramide, dipipanone
antibiotics—aminoglycosides (e.g., amikacin, gentamicin, kanamycin, neomycin, netilmicin, tobramycin, and paromycin), carbapenems (e.g., ertapenem, doripenem, imipenem, cilastatin, and meropenem), cephalosporins (e.g., cefadroxil, cefazolin, cefalotin, cephalexin, cefaclor, cefamandole, cefoxitin, cefprozil, cefuroxime, cefixime, cefdinir, cefditoren, cefoperazone, cefotaxime, cefpodoxime, ceftazidime, ceftibuten, ceftizoxime, ceftriaxone, cefepime, and cefobiprole), glycopeptides (e.g., teicoplanin, vancomycin, and telavancin), lincosamides (e.g., clindamycin and incomysin), lipopeptides) e.g., daptomycin), macrolides (azithromycin, clarithromycin, dirithromycin, erythromycin, roxithromycin, troleandomycin, telithromycin, and spectinomycin), monobactams (e.g., aztreonam), nitrofurans (e.g., furazolidone and nitrofurantoin), penicilllins (e.g., amoxicillin, ampicillin, azlocillin, carbenicillin, cloxacillin, dicloxacillin, flucloxacillin, mezlocillin, methicillin, nafcillin, oxacillin, penicillin G, penicillin V, piperacillin, temocillin, and ticarcillin), penicillin combinations (e.g., amoxicillin/clavulanate, ampicillin/sulbactam, piperacillin/tazobactam, and ticarcillin/clavulanate), polypeptides (e.g., bacitracin, colistin, and polymyxin B), quinolones (e.g., ciprofloxacin, enoxacin, gatifloxacin, levofloxacin, lomefloxacin, moxifloxacin, nalidixic acid, norfloxacin, ofloxacin, trovafloxacin, grepafloxacin, sparfloxacin, and temafloxacin), sulfonamides (e.g., mafenide, sulfonamidochrysoidine, sulfacetamide, sulfadiazine, silver sulfadiazine, sulfamethizole, sulfamethoxazole, sulfanilimide, sulfasalazine, sulfisoxazole, trimethoprim, and trimethoprim-sulfamethoxaxzole), tetracyclines (e.g., demeclocycline, doxycycline, minocycline, oxytetracycline, and tetracycline), antimycobacterial compounds (e.g., clofazimine, dapsone, capreomycin, cycloserine, ethambutol, ethionamide, isoniazid, pyrazinamide, rifampicin (rifampin), rifabutin, rifapentine, and streptomycin), and others. such as arsphenamine, chloramphenicol, fosfomycin, fusidic acid, linezolid, metronidazole, mupirocin, platensimycin, quinuprisin/dalfopristin, rifaximin, thiamphenicol, tigecycline, and tinidazole.
anticoagulants—warfarin (Coumadin®), acenocoumarol, phenprocoumon, atromentin, phenindione, heparin, fondaparinux, idraparinux, rivaroxaban, apixaban, hirudin, lepirudin, bivalirudin, argatrobam, dabigatran, ximelagatran, batroxobin, hementin
anti-delirium agents haloperidol
anti-inflammatory agents—steroids, nonsteroidal anti-inflammatory agents, e.g., cyclooxygenase inhibitors (COX-2 inhibitors, such as rofecoxib, celecoxib), ketoprofen, piroxicam
bronchodilators—albuterol, pirbuterol, metaproterenol, ipratropium, tiotropium, theophylline, salmeterol, formoterol
hypnotics/sedatives—benzodiazepines (alprazolam, chlordiazepoxide, estazolam, flunitrazepam, lormetazepam, midazolam, nitrazepam, oxazepam, quazepam, temazepam, triazolam), barbiturates (amobarbital, pentobarbital, phenobarbital, secobarbital, sodium thiopental), eszopiclone, zaleplon, zolpidem, zopiclone, clomethiazol, antihistamines (diphenhydramine, doxylamine, hydroxyzine, promethazine)
neuromuscular blocking drugs (paralytics) —atracurium (Tracrium®), cisatracurium (Nimbex®), doxacurium (Nuromax®), pancuronium bromide (Pavulon®), pipecuronium (Arduan®), tuboccurarine (Tubarine®), vecuronium (Norcuron®), mivacurium (Mivacron®), rapacuronium (Raplon®), rocuronium (Zemuron®), succinyocholine (Anectine®).

E. Methods of Administration

The disclosed compounds can be administered by oral, parenteral (e.g., intramuscular, intraperitoneal, intravenous, ICV, intracisternal injection or infusion, subcutaneous injection, or implant), by inhalation spray nasal, vaginal, rectal, sublingual, urethral (e.g., urethral suppository) or topical routes of administration (e.g., gel, ointment, cream, aerosol, etc.) and can be formulated, alone or together, in suitable dosage unit formulations containing conventional non-toxic pharmaceutically acceptable carriers, adjuvants, excipients, and vehicles appropriate for each route of administration. In addition to the treatment of warm-blooded animals such as mice, rats, horses, cattle, sheep, dogs, cats, and monkeys, the compounds described herein can be effective in humans. For topical administration, the inhibitors of muscle degradation, or prodrugs thereof, can be formulated as solutions, gels, ointments, creams, jellies, solutions, suspensions, etc., as are well-known in the art. Such formulations can be included in a patch or other transdermal delivery system or formulation, e.g., a formulation with ingredients specifically designed to aid transport of the compound through the skin and into the body tissues. In certain embodiments, the compounds can be formulated for topical administration with polyethylene glycol (PEG). These formulations can optionally include additional pharmaceutically acceptable ingredients such as diluents, stabilizers, and/or adjuvants.

Systemic formulations include those designed for administration by injection (e.g., subcutaneous, intravenous, intramuscular, intrathecal, or intraperitoneal injection) as well as those designed for transdermal, transmucosal, oral, or pulmonary administration. Pharmaceutically acceptable excipients may be used in combination with disclosed embodiments of the compound. Excipients include, but are not limited to, buffers, tonic agents, and the like.

Useful injectable preparations include sterile suspensions, solutions, or emulsions of the active compound(s) in aqueous or oily vehicles. The compositions can also contain formulating agents, such as suspending, stabilizing, and/or dispersing agents. The formulations for injection can be presented in unit dosage form, e.g., in ampules or in multidose containers, and can contain added preservatives.

Alternatively, the injectable formulation can be provided in powder form for reconstitution with a suitable vehicle, including but not limited to sterile pyrogen free water, buffer, and dextrose solution, before use. To this end, the active compound(s) can be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions can take the form of for example, lozenges, tablets, or capsules prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulfate). The tablets can be coated by methods well known in the art with, for example, sugars, films, or enteric coatings. Additionally, the pharmaceutical compositions containing the compound as active ingredient or prodrug thereof in a form suitable for oral use can also include, for example, troches, lozenges, aqueous, or oily suspensions, dispersible powders or granules, emulsions, hard or soft capsules, or syrups or elixirs. Compositions intended for oral use can be prepared according to any method known to the art for the manufacture of pharmaceutical compositions, and such compositions can contain one or more agents including sweetening agents, flavoring agents, coloring agents, and preserving agents in order to provide pharmaceutically elegant and palatable preparations. Tablets contain the active ingredient (including drug and/or prodrug) in admixture with non-toxic pharmaceutically acceptable excipients which are suitable for the manufacture of tablets. These excipients can be for example, inert diluents, such as calcium carbonate, sodium carbonate, lactose, calcium phosphate or sodium phosphate; granulating and disintegrating agents (e.g., corn starch or alginic acid); binding agents (e.g. starch, gelatin, or acacia); and lubricating agents (e.g., magnesium stearate, stearic acid, or talc). The tablets can be left uncoated or they can be coated by known techniques to delay disintegration and absorption in the gastrointestinal tract and thereby provide a sustained action over a longer period. For example, a time delay material such as glyceryl monostearate or glyceryl distearate can be employed. They can also be coated by the techniques described in the U.S. Pat. Nos. 4,256,108; 4,166,452; and 4,265,874 to form osmotic therapeutic tablets for control release. The pharmaceutical compositions described herein can also be in the form of oil-in-water emulsions.

Liquid preparations for oral administration can take the form of, for example, elixirs, solutions, syrups, or suspensions, or they can be presented as a dry product for constitution with water or other suitable vehicle before use. Such liquid preparations can be prepared by conventional means with pharmaceutically acceptable additives such as suspending agents (e.g., sorbitol syrup, cellulose derivatives, or hydrogenated edible fats); emulsifying agents (e.g., lecithin, or acacia); non-aqueous vehicles (e.g., almond oil, oily esters, ethyl alcohol, Cremophore™, or fractionated vegetable oils); and preservatives (e.g., methyl or propyl-p-hydroxybenzoates or sorbic acid). The preparations can also contain buffer salts, preservatives, flavoring, coloring, and sweetening agents as appropriate.

Preparations for oral administration can be suitably formulated to give controlled release of the active compound, as is well known.

For buccal administration, the compositions can take the form of tablets or lozenges formulated in the conventional manner.

For nasal administration or administration by inhalation or insufflation, the active compound(s) or prodrug(s) can be conveniently delivered in the form of an aerosol spray from pressurized packs or a nebulizer with the use of a suitable propellant (e.g., dichlorodifluoromethane, trichlorofluoromethane, dichlorotetrafluoroethane, fluorocarbons, carbon dioxide, or other suitable gas). In the case of a pressurized aerosol, the dosage unit can be determined by providing a valve to deliver a metered amount. Capsules and cartridges for use in an inhaler or insufflator (for example, capsules and cartridges including gelatin) can be formulated containing a powder mix of the compound and a suitable powder base such as lactose or starch.

The pharmaceutical compositions can be in the form of a sterile injectable aqueous or oleaginous suspension. This suspension can be formulated according to the known art using those suitable dispersing or wetting agents and suspending agents which have been mentioned above. The sterile injectable preparation can also be a sterile injectable solution or suspension in a non-toxic parenterally-acceptable diluent or solvent. Among the acceptable vehicles and solvents that can be employed are water, Ringer's solution, and isotonic sodium chloride solution. The disclosed compounds can also be administered in the form of suppositories for rectal or urethral administration of the drug. For rectal and vaginal routes of administration, the active compound(s) can be formulated as solutions (for retention enemas), suppositories, or ointments containing conventional suppository bases such as cocoa butter or other glycerides. In particular embodiments, the compounds can be formulated as urethral suppositories, for example, for use in the treatment of fertility conditions, particularly in males (e.g., for the treatment of testicular dysfunction).

The disclosed compounds can be used for manufacturing a composition or medicament, including medicaments suitable for rectal or urethral administration. Also specifically contemplated herein are methods for manufacturing compositions including the presently disclosed compounds in a form that is suitable for urethral or rectal administration, including suppositories.

The presently disclosed compounds can be used for manufacturing a composition or medicament, including medicaments suitable for topical administration. Accordingly, specifically contemplated are methods for manufacturing compositions including compounds in a form that is suitable for topical administration.

For ocular administration, the compound(s) or prodrug(s) can be formulated as a solution, emulsion, suspension, etc., suitable for administration to the eye. Administration to the eye is generally via topical exposure of the eye to the formulation, but also includes injection into the eye if necessary. A variety of vehicles suitable for administering compounds to the eye are known in the art. Specific non-limiting examples are described in U.S. Pat. No. 6,261,547; U.S. Pat. No. 6,197,934; U.S. Pat. No. 6,056,950; U.S. Pat. No. 5,800,807; U.S. Pat. No. 5,776,445; U.S. Pat. No. 5,698,219; U.S. Pat. No. 5,521,222; U.S. Pat. No. 5,403,841; U.S. Pat. No. 5,077,033; U.S. Pat. No. 4,882,150; and U.S. Pat. No. 4,738,851.

Typically formulations for ocular administration contain a pharmaceutically effective amount of a compound disclosed herein, such as from about 0.0001% to about 1.0% by weight (w/w). In certain formulations, the pharmaceutically effective amount of the compound is 0.0003% to about 0.1% (w/w), such as from about 0.003% to about 0.5% (w/w), or from about 0.01% to about 0.03% (w/w).

In certain examples an ophthalmic composition containing a presently disclosed compound for ocular administration includes a tonicity agent, a buffer, or both. In certain examples of ophthalmic compositions the tonicity agent is a simple carbohydrate or a sugar alcohol. As is known to those of ordinary skill in the art, tonicity agents may be used in the present compositions to adjust the tonicity of the composition, preferably to that of normal tears. Examples of suitable tonicity agents include, without limitation sodium chloride, potassium chloride, magnesium chloride, calcium chloride, carbohydrates, such as dextrose, fructose, galactose, polyols, such as sugar alcohols, including by way of example, mannitol, sorbitol, xylitol, lactitol, isomalt, maltitol and combinations thereof. Compositions containing a buffer contain, in some examples, a phosphate, citrate, or both.

In one aspect, compositions for ocular administration of the presently disclosed compounds optionally contain a surfactant, a stabilizing polymer, or both. Surfactants are employed in certain compositions to facilitate the delivery of higher concentrations of the compound being administered. Such surfactants can work to solubilize the compound. Exemplary surfactants include polysorbate, poloxamer, polyoxyl 40 stearate, polyoxyl castor oil, tyloxapol, triton and sorbitan monolaurate. In certain embodiments the surfactant is selected from Triton X114, tyloxapol and combinations thereof. In still another embodiment of compositions for ocular administration, the stabilizing polymer is carbomer 974p.

For prolonged delivery, the compound(s) or prodrug(s) can be formulated as a depot preparation for administration by implantation or intramuscular injection. The active ingredient can be formulated with suitable polymeric or hydrophobic materials (e.g., as an emulsion in an acceptable oil) or ion exchange resins, or as sparingly soluble derivatives (e.g., as a sparingly soluble salt). Alternatively, transdermal delivery systems manufactured as an adhesive disc or patch which slowly releases the active compound(s) for percutaneous absorption can be used. To this end, permeation enhancers can be used to facilitate transdermal penetration of the active compound(s). Suitable transdermal patches are described in, for example, U.S. Pat. No. 5,407,713; U.S. Pat. No. 5,352,456; U.S. Pat. No. 5,332,213; U.S. Pat. No. 5,336,168; U.S. Pat. No. 5,290,561; U.S. Pat. No. 5,254,346; U.S. Pat. No. 5,164,189; U.S. Pat. No. 5,163,899; U.S. Pat. No. 5,088,977; U.S. Pat. No. 5,087,240; U.S. Pat. No. 5,008,110; and U.S. Pat. No. 4,921,475.

Alternatively, other pharmaceutical delivery systems can be employed. Liposomes and emulsions are well-known examples of delivery vehicles that can be used to deliver active compound(s) or prodrug(s). Certain organic solvents such as dimethylsulfoxide (DMSO) can also be employed, although usually at the cost of greater toxicity.

The pharmaceutical compositions can, if desired, be presented in a pack or dispenser device which can contain one or more unit dosage forms containing the active compound(s). The pack can, for example, include metal or plastic foil, such as a blister pack. The pack or dispenser device can be accompanied by instructions for administration.

### III. Method of Use

Without being limited to a theory of operation, it is currently believed that muscle degradation is an inflammatory response mediated by Janus kinases. Compounds disclosed herein are effective kinase inhibitors, particularly Janus kinase inhibitors. Embodiments of the disclosed compound may be used to treat muscular degradation, such as atrophy, dystrophy, or cachexia in a subject suffering from any disease causing these conditions. Embodiments of the disclosed compound may also be used to treat a subject suffering from atrophy resulting from disuse or inactivity. Additionally, embodiments of the disclosed compound may be used to treat atrophy caused by controlled mechanical ventilation (CMV), including ventilator disuse atrophy, and ventilator-induced diaphragmatic dysfunction.

Particular examples of diseases that may be treated using embodiments of the disclosed compound, or compositions thereof include, but are not limited to, spinal muscular atrophy, or “SMA” (e.g. SMA type 1, SMA type 2, SMA type 3, and SMA type 4), multiple system atrophy (e.g. Shy-Drager syndrome, sporadic olivopontocerebellar atrophy, progressive autonomic failure with multiple system atrophy, sporadic OPCA, striatonigral degeneration), muscular atrophy and/or cachexia resulting from common diseases (e.g. cancer, AIDS, renal failure, sarcopenia), and dystrophic disorders (e.g. Duchenne, myotonic, Becker, Kearns-Sayre syndrome, Leigh's syndrome, limb-girdle, facioscapulohumeral, congenital, oculopharyngeal, distal, and Emery-Dreifuss dystrophy). Certain atrophic and/or dystrophic disorders can result from disorders of the vascular and/or circulatory systems, such as congestive heart failure, chronic obstructive pulmonary disease, and peripheral artery disease.

The disclosed compound(s) or prodrug(s) described herein, or compositions thereof, will generally be used in an amount effective to achieve the intended result, for example, in an amount effective to treat or prevent the particular condition being treated. The compound(s) can be administered therapeutically to achieve therapeutic benefit or prophylactically to achieve prophylactic benefit. “Therapeutic benefit” means eradicating or amerliorating the underlying disorder being treated and/or eradication or amelioration of one or more of the symptoms associated with the underlying disorder such that the subject reports an improvement in feeling or condition, notwithstanding that the subject may still be afflicted with the underlying disorder. The amount of compound administered will depend upon a variety of factors, including, for example, the particular condition being treated, the mode of administration, the severity of the condition being treated, the age, weight and general health of the subject, the bioavailability of the particular active compound, and whether the compound is administered prophylactically. Determination of an effective dosage is well within the capabilities of those having ordinary skill in the art.

For prophylactic administration, the compound can be administered to a subject at risk of developing one of the previously described conditions. For example, if it is unknown whether a subject is susceptible to muscular atrophy, dystrophy, or cachexia, the compound can be administered prior to the onset of symptoms resulting from these conditions. Alternatively, prophylactic administration can be applied to avoid the onset of symptoms in a subject diagnosed with the underlying disorder. For example, a compound can be administered to a genetically predisposed subject prior to expected onset of the disease, such as in the case of dystrophy.

The amount of compound administered will depend upon a variety of factors, including, for example, the particular indication being treated, the mode of administration, whether the desired benefit is prophylactic or therapeutic, the severity of the indication being treated and the age and weight of the subject, and the bioavailability of the particular active compound. Determination of an effective dosage is well within the capabilities of those having ordinary skill in the art.

Effective dosages can be estimated initially from in vitro assays. For example, an initial dosage for use in animals can be formulated to achieve a circulating blood or serum concentration of active compound that is at or above an IC₅₀ of the particular compound as measured in an in vitro assay. Calculating dosages to achieve such circulating blood or serum concentrations taking into account the bioavailability of the particular compound is well within the capabilities of those having ordinary skill in the art. For guidance, the reader is referred to Fingl & Woodbury, “General Principles,” In: Goodman and Gilman's The Pharmaceutical Basis of Therapeutics, Chapter 1, 12th edition, Pergamon Press, and the references cited therein.

Initial dosages can also be estimated from in vivo data, such as animal models. Animal models useful for testing the efficacy of compounds to treat or prevent the various diseases described above are well-known in the art. Suitable animal models of hypersensitivity or allergic reactions are described in Foster, (1995) Allergy 50(21Suppl):6-9, discussion 34-38 and Tumas et al., (2001), J. Allergy Clin. Immunol. 107(6):1025-1033. Suitable animal models of allergic rhinitis are described in Szelenyi et al., (2000), Arzneimittelforschung 50(11):1037-42; Kawaguchi et al., (1994), Clin. Exp. Allergy 24(3):238-244 and Sugimoto et al., (2000), Immunopharmacology 48(1):1-7. Suitable animal models of allergic conjunctivitis are described in Carreras et al., (1993), Br. J. Ophthalmol. 77(8):509-514; Saiga et al., (1992), Ophthalmic Res. 24(1):45-50; and Kunert et al., (2001), Invest. Ophthalmol. Vis. Sci. 42(11):2483-2489. Suitable animal models of systemic mastocytosis are described in O'Keefe et al., (1987), J. Vet. Intern. Med. 1(2):75-80 and Bean-Knudsen et al., (1989), Vet. Pathol. 26(1):90-92. Suitable animal models of hyper IgE syndrome are described in Claman et al., (1990), Clin. Immunol. Immunopathol. 56(1):46-53. Suitable animal models of B-cell lymphoma are described in Hough et al., (1998), Proc. Natl. Acad. Sci. USA 95:13853-13858 and Hakim et al., (1996), J. Immunol. 157(12):5503-5511. Suitable animal models of atopic disorders such as atopic dermatitis, atopic eczema, and atopic asthma are described in Chan et al., (2001), J. Invest. Dermatol. 117(4):977-983 and Suto et al., (1999), Int. Arch. Allergy Immunol. 120(Suppl 1):70-75. Suitable animal models of transplant rejection, such as models of HVGR, are described in O'Shea et al., (2004), Nature Reviews Drug Discovery 3:555-564; Cetkovic-Curlje & Tibbles, (2004), Current Pharmaceutical Design 10:1767-1784; and Chengelian et al., (2003), Science 302:875-878. Persons of ordinary skill in the art can routinely adapt such information to determine dosages suitable for human administration.

Dosage amounts will typically be in the range of from about 0.0001 or 0.001 or 0.01 mg/kg/day to about 100 mg/kg/day, but can be higher or lower, depending upon, among other factors, the activity of the compound, its bioavailability, the mode of administration, and various factors discussed above. In one embodiment the daily dosage may be greater than zero milligrams per day, such as from about 1 mg/day, up to at least about 2 grams/day. More typically, the dosage (or effective amount) may range from about 2 mg/kg/day, about 3 mg/kg/day, about 5 mg/kg/day, about 10 mg/kg/day, about 15 mg/kg/day, about 20 mg/kg/day or about 50 mg/kg/day. Dosage amount and interval can be adjusted individually to provide plasma levels of the compound(s) sufficient to maintain therapeutic or prophylactic effect. For example, the compounds can be administered once per week, several times per week (e.g., every other day), once per day, or multiple times per day, depending upon, among other things, the mode of administration, the specific indication being treated, and the judgment of the prescribing physician. In cases of local administration or selective uptake, such as local topical administration, the effective local concentration of active compound(s) may not be related to plasma concentration. Persons of ordinary skill in the art will be able to optimize effective local dosages without undue experimentation.

Preferably, the compound(s) will provide therapeutic or prophylactic benefit without causing substantial toxicity. Toxicity of the compound(s) can be determined using standard pharmaceutical procedures. The dose ratio between toxic and therapeutic (or prophylactic) effect is the therapeutic index. Compounds(s) that exhibit high therapeutic indices are preferred.

The foregoing disclosure pertaining to the dosage requirements for the compounds is pertinent to dosages required for prodrugs, with the realization, apparent to a person of ordinary skill in the art, that the amount of prodrug(s) administered will also depend upon a variety of factors, including, for example, the bioavailability of the particular prodrug(s) and the conversation rate and efficiency into active drug compound under the selected route of administration. Determination of an effective dosage of prodrug(s) for a particular use and mode of administration is well within the capabilities of those having ordinary skill in the art.

Effective dosages can be estimated initially from in vitro activity and metabolism assays. For example, an initial dosage of prodrug for use in animals can be formulated to achieve a circulating blood or serum concentration of the metabolite active compound that is at or above an IC₅₀ of the particular compound as measured in an in vitro assay, such as the in vitro CHMC or BMMC and other in vitro assays described in U.S. application Ser. No. 10/355,543 filed Jan. 31, 2003 (US2004/0029902A1, U.S. Pat. No. 7,557,210), international application Serial No. PCT/US03/03022 filed Jan. 31, 2003 (WO 03/063794), U.S. application Ser. No. 10/631,029 filed Jul. 29, 2003 (U.S. Pat. No. 7,517,886), international application Serial No. PCT/US03/24087 (WO2004/014382), U.S. application Ser. No. 10/903,263 filed Jul. 30, 2004 (U.S. Pat. No. 8,178,671), and international application Serial No. PCT/US2004/24716 (WO005/016893). Calculating dosages to achieve such circulating blood or serum concentrations, taking into account the bioavailability of the particular prodrug via the desired route of administration, is well within the capabilities of persons having ordinary skill in the art. For guidance, the reader is referred to Fingl & Woodbury, “General Principles,” In: Goodman and Gilman's The Pharmaceutical Basis of Therapeutics, Chapter 1, 12^{th} edition, Pagamonon Press, and the references cited therein.

Particular disclosed embodiments concern a method, comprising administering to a subject one or more compounds, other than a peptide-based compound (e.g. leupeptin), in an amount effective to inhibit or prevent atrophy, dystrophy, or cachexia. The one or more compounds may be a pyrimidine- or pyridine-containing compound.

In particular disclosed embodiments, administering comprises exposing the subject to a dosage of the compound that is adjusted to inhibit or prevent muscular deterioration, which includes muscle injury, muscle weakness, and loss of muscle mass. The subject may suffer from atrophy, dystrophy, or cachexia, and the atrophy may be the result of disuse or inactivity. In particular disclosed embodiments, the atrophy and/or cachexia is caused by a disease selected from cancer, AIDS, renal failure, sarcopenia, or combinations thereof. In subjects suffering from dystrophy, the dystrophy may be caused by a dystrophic disorder, such as, but not limited to Duchenne dystrophy, myotonic dystrophy, Becker dystrophy, limb-girdle dystrophy, facioscapulohumeral dystrophy, congenital dystrophy, oculopharyngeal dystrophy, distal dystrophy, Emery-Dreifuss dystrophy, or combinations thereof. The subject may be a trauma patient or a surgical patient, particularly trauma or surgical patients that require or are likely to require mechanically assisted breathing such as a ventilator. In particular disclosed embodiments, the subject is not a transplant recipient. In yet other embodiments, the subject is not a transplant recipient and the compound that is administered to the subject is not 3-{(3R,4R)-4-methyl-3-[methyl-(7H-pyrrolo[2,3-d]pyrimidin-4-yl)-amino]-piperidin-1-yl}-3-oxo-propionitrile or a salt, solvate or prodrug thereof.

The method may further comprise monitoring the level of muscular deterioration in the subject to ascertain the effect of the compound. The compound also may be administered alone or as a pharmaceutical composition and typically is administered intravenously. Additionally, the compound may be administered prophylactically.

Other disclosed embodiments concern a method for treating muscular deterioration, comprising administering to a subject diagnosed with a dystrophic disorder a compound in an amount effective to improve the whole body strength of the subject, wherein the compound is selected from any one of the compounds disclosed herein. The method may further comprise monitoring the whole body strength of the subject in order to determine the ability of the compound to inhibit and/or prevent muscular deterioration.

In particular disclosed embodiments, a method for inhibiting or preventing muscular deterioration is contemplated, wherein the method comprises diagnosing a subject in need of treatment for muscular deterioration, administering to the subject a compound in an amount effective to inhibit and/or prevent muscular deterioration, the compound being selected from any one of the compounds disclosed herein, and permitting the compound to achieve therapeutic benefit for muscular deterioration in the subject.

In particular disclosed embodiments, the method comprises administering one or more disclosed embodiments of the compound, or compositions thereof, (and excluding leupeptin) to a subject in an amount effective to inhibit or prevent atrophy, dystrophy, or cachexia. In particular disclosed embodiments, the one or more compounds is a pyrimidine-containing compound, a pyridine-containing compound, or combinations thereof. The compounds may have any one of the formulas provided herein.

Typically, administering comprises exposing the subject to at least a first dose of the compound, or composition comprising the compound. The method may further comprise determining a therapeutic blood level of the one or more compounds in the subject, or a therapeutic metabolite blood level of the one or more compounds, in the subject. Additionally, the method may comprise, after determining the therapeutic blood level, adjusting the first dose to a second dose, such as to optimize therapeutic effect. In particular disclosed embodiments, the method further comprises monitoring muscular deterioration in the subject to ascertain the ability of the compound to inhibit or prevent muscular degradation, such as atrophy, dystrophy, or cachexia. The method may further comprise monitoring the whole body strength of the subject in order to determine the ability of the one or more compounds to inhibit muscular deterioration. A single compound may be administered serially in plural administrations to the subject, or two or more compounds may be administered either serially or in combination to the subject. The compound can be administered continuously. In particular disclosed embodiments, the one or more compounds are administered as a pharmaceutical composition. The method concerns methods of administration including oral, buccal, mucosal, sublingual, intravenous, intra-arterial, intramuscular, subcutaneous, intraperitoneal, intraarticular, infusion, intrathecal, intraurethral, topical, subdermal, transdermal, intranasal, inhalation, pulmonary tract, intratracheal, intraocular, ocular, intraaural, vaginal, and rectal. In particular disclosed embodiments, the compound is administered intratraceally, intravenously, and/or prophylactically.

The method may also comprise administering a second therapeutic agent to the subject. The second therapeutic may be selected from an analgesic, an antibiotic, an anticoagulant, an anti-delirium agent, an anti-inflammatory, a bronchodilator, a hypnotic, a sedative, a neuromuscular blocking agent, an antihistamine, or combinations thereof. In particular disclosed embodiments, the second therapeutic agent is administered prior to or subsequent to the one or more compounds. In other embodiments, the second therapeutic agent is administered in combination with the one or more compounds. The second therapeutic agent may be selected from any of those disclosed herein.

Particular disclosed embodiments concern a method wherein the subject is or is about to be placed on a ventilator. The subject may also be experiencing ventilator-induced diaphragmatic dysfunction.

In particular disclosed embodiments, the method concerns inhibiting or preventing muscular deterioration, comprising, wherein the method comprises: diagnosing a subject as being in need of treatment for muscular deterioration; administering to the subject one or more compounds in an amount effective to inhibit muscular deterioration, the one or more compounds being selected from any one of the compounds disclosed herein; and evaluating the subject to determine future course of treatment.

Further contemplated is a method for treating ventilator-induced diaphragmatic dysfunction, comprising administering to a subject one or more compounds in an amount effective to reduce or inhibit injury of diaphragm muscle fibers, the one or more compounds being selected from any one of the compounds disclosed herein. These embodiments may further comprise exposing the subject to a first dosage of the one or more compounds; and adjusting the first dose to a second dose optimized to inhibit and/or prevent injury of diaphragm muscle fibers. In particular disclosed embodiments, the one or more compounds are administered before the subject is ventilated, while ventilated, after the subject is ventilated, or prior to weaning the subject off a ventilator at a time period effective to reduce muscular degradation, such as atrophy, dystrophy, or cachexia. Also, the compound may be administered to the subject prior to weaning the subject off a ventilator. In particular disclosed embodiments, the compound is administered to a subject to reduce the time required to wean the subject from a ventilator, such as reducing a weaning time to a period of fewer than three days. The compound may be administered as a pharmaceutical composition, and typically is administered intravenously.

Other embodiments of the disclosed method concern treating cancer cachexia in a subject. In these embodiments, the methods comprises administering to the subject a compound in an amount effective to inhibit and/or prevent cancer cachexia, the compound being selected from any one of the compounds disclosed herein. The method may comprise an administration step comprising exposing the subject to a dosage of the compound that is adjusted to inhibit and/or prevent cancer cachexia. As in other embodiments, the compound may be administered as a pharmaceutical composition, and typically is administered intravenously. The compound may also be administered prophylactically.

### IV. Examples

The following examples may be used to enable a person of ordinary skill in the art in practicing the disclosed method. A person of ordinary skill in the art will recognize that the following example may be adapted as needed based on the results obtained from using the disclosed method.

A. Test Groups

Appropriate test groups are selected to determine the ability of embodiments of the disclosed compound, or compositions thereof, to reduce and/or prevent muscular degradation, such as muscle atrophy resulting from ventilation. Three groups are selected, wherein the first group comprises an acutely anesthetized control group; the second group comprises an anesthetized group submitted to CMV for a period of time (e.g. 24 hours) and receiving a single intramuscular injection of saline; and the third group comprises subjects that have been anesthetized and submitted to CMV for a period of time (e.g. 24 hours) and receiving a single intramuscular injection of an effective amount of a disclosed embodiment of the compound, or composition thereof. The effective amount of the compound may be administered prior to the start of CMV, at the start of CMV, during CMV, or after CMV. Typically, a disclosed embodiment of the compound is administered at the start of CMV.

B. Mechanical Ventilation Protocol

Animal subjects are anesthetized with an intraperitoneal injection of an appropriate anaesthetic, such as sodium pentobarbital, and then tracheostomized. The animal subjects are then mechanically ventilated at a suitable breathing frequency, such as about 60 breaths per minute, with a ventilator for approximately 24 hours. The animal subjects are provided air during ventilation, preferably humidified and oxygenated air, as well as a continuous dose of anesthesia and an appropriate anticoagulant, such as heparin. In particular embodiments, the dose of anesthesia may range from about 0.5 mg/100 g/h to about 5 mg/100 g/h; more typically from about 1 mg/100 g/h to about 4 mg/100 g/h; even more typically from about 2 mg/100 g/h to about 3 mg/100 g/h. The body temperature of the animal subjects is continuously maintained at approximately 37° C. Appropriate monitoring may also be conducted, such as that necessary to monitor arterial blood pressure and blood gasses, at any time during CMV.

C. Determination of Efficacy

After a suitable amount of time on the ventilator, the effect of the disclosed compound on the animal subjects is determined using the following suggested protocol. Segments of costal diaphragm muscle are removed from the animal subjects in order to measure the in vitro contractile properties of the muscle, using methods known to those of ordinary skill in the art (see, for example, Gayan-Ramirez G, Testelmans D, Maes K, Racz G Z, Cadot P, Zador E, Wuytack F, Decramer M. “Intermittent spontaneous breathing protects the rat diaphragm from mechanical ventilation effects. Crit Care Med 2005; 33:2804-2809).

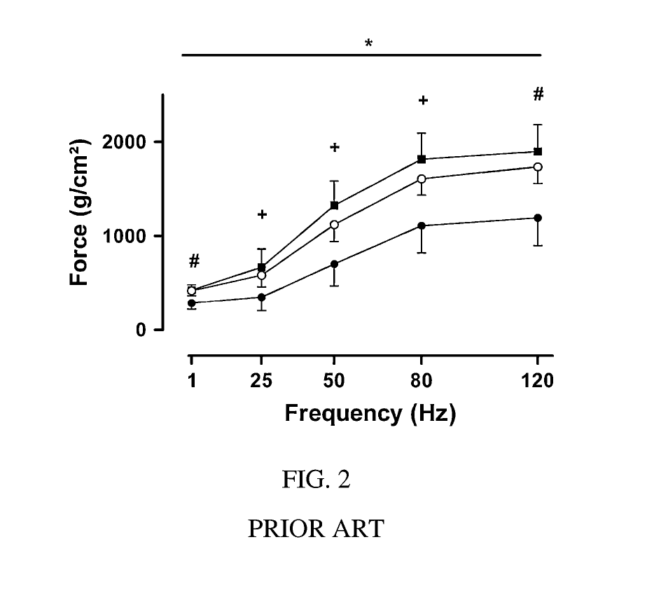

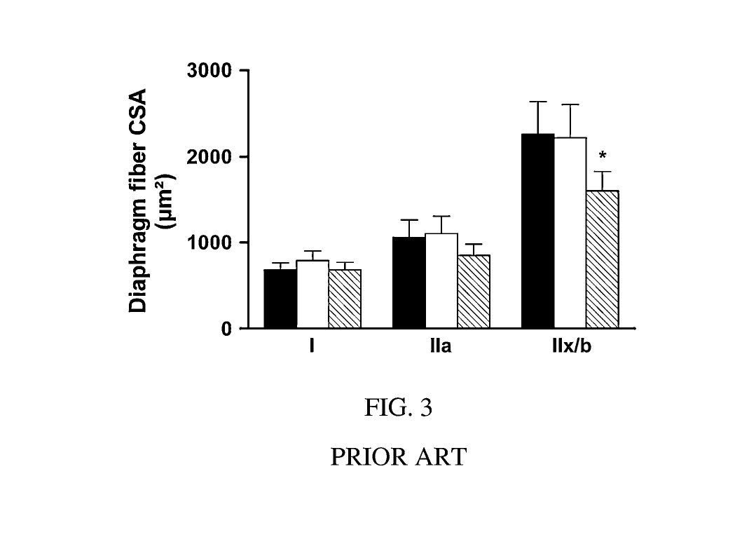

Various types of analyses may be carried out on the segmented diaphragm tissue in order to assess the effect of the disclosed compound, or composition thereof, on atrophy resulting from CMV. Histology and histochemical analysis may conducted on the diaphragm segments in order to determine the ability of the disclosed compounds, or compositions thereof, to inhibit or suppress diaphragm atrophy resulting from ventilation. See, for example, FIG. 2, which illustrates results obtained in the prior art using force-frequence analysis of diaphragm segments taken from subjects (Maes, et al. “Leupeptin Inhibits Ventilator-induced Diaphragm Dysfunction in Rats,” American Journal of Respiratory and Critical Care Medicine, vol. 175, 2007). In particular disclosed embodiments, histology and histochemical analysis may be carried out in order to determine structural abnormalities and atrophy of particular muscle fibers within the diaphragm. See, for example, FIG. 3, which illustrates results obtained in the prior art using this method (Maes, et al.).

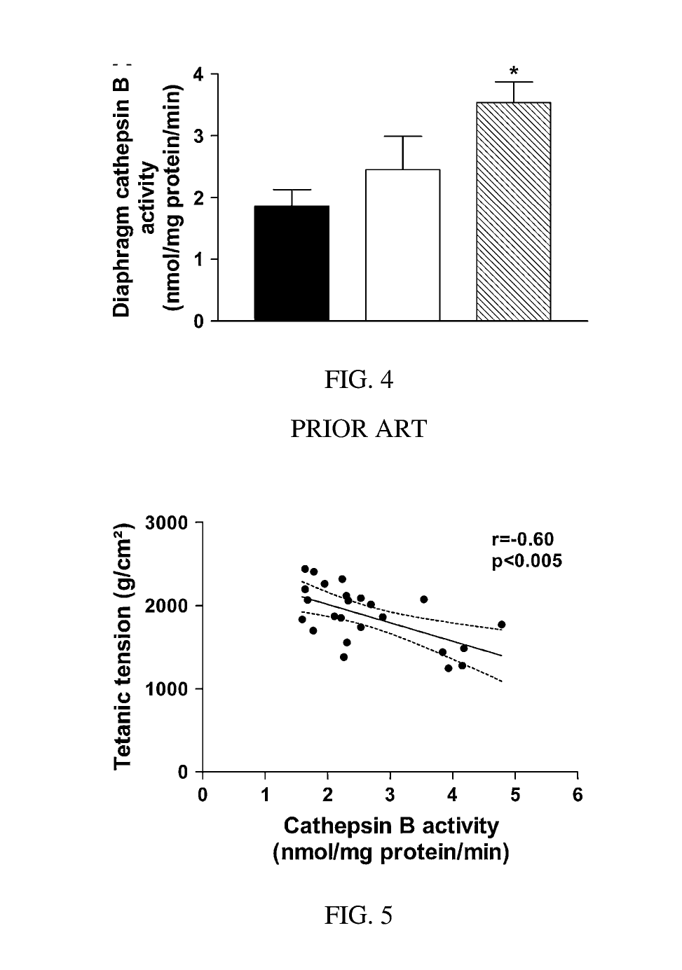

Also, in vitro diaphragm cathepsin B activity may be measured using methods known to a person of ordinary skill in the art, such as by using a cathepsin B activity assay kit. See, for example, FIGS. 4 and 5, which illustrate results obtained in the prior art using this method (Maes, et al.).

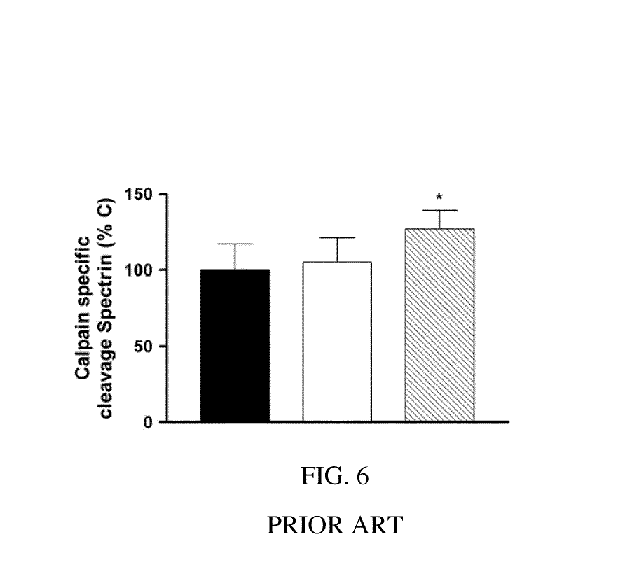

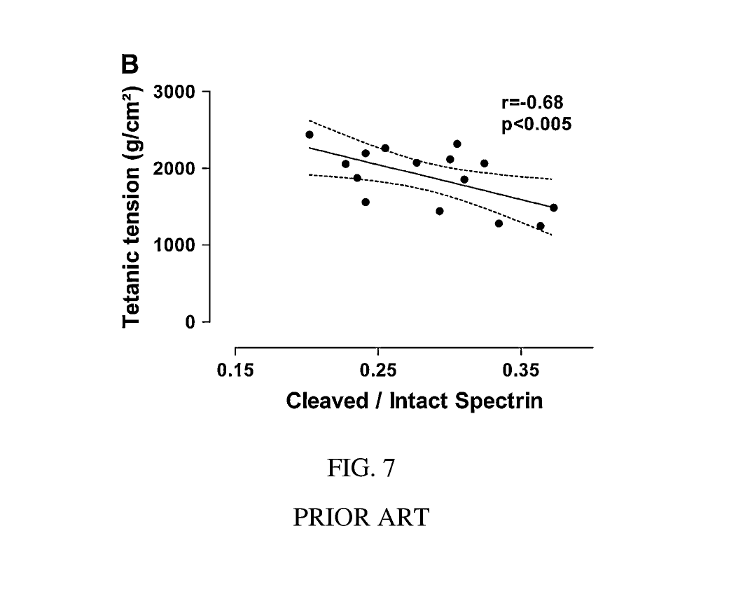

Another measurement technique that is used involves assessing diaphragm calpain activity. Such measurement may involve detecting products of aII-spectrin cleavage using antibody detection techniques and Western blotting. See, for example, FIGS. 6 and 7, which illustrate results obtained in the prior art using this technique.

A person of ordinary skill in the art may use these different techniques in order to assess the ability of the disclosed compound, or compositions thereof, to inhibit or decrease the amount of muscular atrophy caused by CMV. For further guidance relating to these methods and the interpretation of results obtained therefrom, see Maes, et al. (cited above), which is incorporated herein by reference.

D. Results

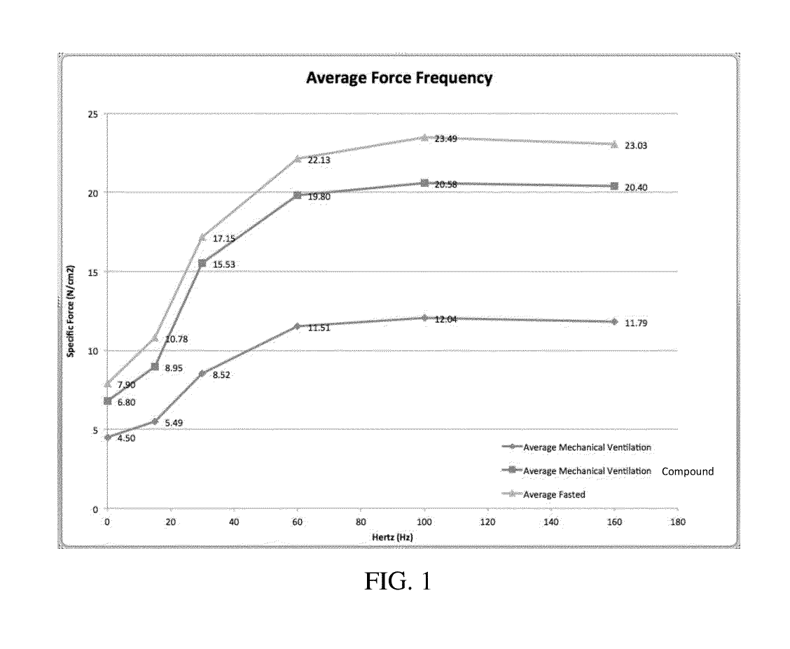

An embodiment of the disclosed compound was shown to effectively inhibit diaphragm muscle loss. FIG. 1 illustrates the effect of the disclosed embodiment on the diaphragm contractility of ventilated rats. According to FIG. 1, ventilated rats on vehicle-only lost nearly 50% of their diaphragm contractility in ex vivo tests, wherein ventilated rats treated with at least one compound disclosed herein were spared approximately 90% of the loss of diaphragm contractility during 18 hours of ventilation. The rats were treated with a constant IV infusion of the particular compound (e.g. 1000 ng/mL plasma). As indicated herein, exemplary compounds exhibit the ability to reduce ventilator-associated atrophy in rat models.

E. Correlation of Animal Models

The animal models described herein provide evidence that mechanical ventilation causes atrophy and impaired contractility of the diaphragm, and it is currently believed that the disclosed compound is effective in inhibiting and/or decreasing the amount of atrophy resulting from CMV. A person of ordinary skill in the art will recognize that the disclosed animal models may be used and/or modified for use in human subjects. A recent article by Jaber et al. illustrates the rapid onset of diaphragmatic weakness and atrophy in ventilated humans, and further illustrates that mechanical ventilation is associated with structural injury to diaphragm muscle fibers and up-regulation of the calpain proteolytic system. Thus, there is an understood correlation in the art between the disclosed animal models and their application in humans.

In particular disclosed embodiments, any one or more of the compounds disclosed herein are administered to a human subject in order to inhibit diaphragm muscle deterioration and therefore promote diaphragm contractility in human subjects. The human subject is ventilated and administered one or more of the compounds disclosed herein; alternatively, the human subject may be administered one or more of the compounds disclosed herein and then ventilated.

In view of the many possible embodiments to which the principles of the disclosed invention may be applied, it should be recognized that the illustrated embodiments are only preferred examples of the invention and should not be taken as limiting the scope of the invention. Rather, the scope of the invention is defined by the following claims. We therefore claim as our invention all that comes within the scope and spirit of these claims.

We claim:

1. A method, comprising administering to a subject a kinase inhibitor in an amount and at a time effective to reduce or prevent muscle degradation.

2. The method according to claim 1 comprising administering to the subject an amount of the kinase inhibitor affective to inhibit JAK1, JAK2, JAK3, TYK2, or a combination thereof.

3. The method according to claim 1 wherein the kinase inhibitor is administered in an amount effective to inhibit or prevent atrophy, dystrophy, or cachexia.

4. The method according to claim 1 comprising administering to a subject requiring controlled mechanical ventilation.

5. The method according to claim 1 wherein the subject is on, or is about to be placed on, a ventilator.

6. The method according to claim 5 wherein the subject is experiencing, or has experienced, ventilator-induced diaphragmatic dysfunction, ventilator disuse atrophy, or both.

7. The method according to claim 1 wherein the kinase inhibitor is a pyrimidine-containing compound, a pyridine-containing compound, or a combination thereof.

8. The method according to claim 1 wherein the kinase inhibitor has a formula selected from the group consisting of: 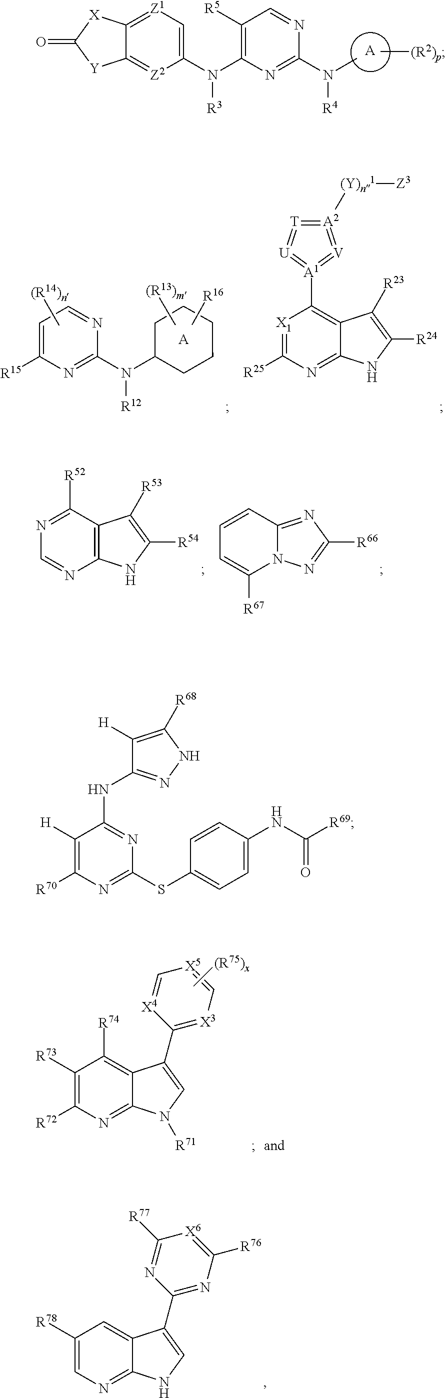
wherein X and Y are each independently O, S, S(O), SO₂ or NR¹;
each R¹ is independently for each occurrence H, optionally substituted C₁₋₆alkyl, C(O)—C₁₋₆alkyl, CO₂—C₁₋₆alkyl or R⁵⁰;
each R⁵⁰ is —C(R⁹)₂-A-R¹⁰, where A is O or S;
each R⁹ is independently for each occurrence H, optionally substituted C₁₋₆alkyl, optionally substituted C₆₋₁₀aryl or optionally substituted C₇₋₁₆arylalkyl; or alternatively, two R⁹, together with the carbon to which they are attached, form an optionally substituted C₃₋₈cycloalkyl group or an optionally substituted 3-8 membered heteroalicyclyl;
R¹⁰ is R^{a} or —P(O)(OR¹¹)₂;
each R¹¹ is independently for each occurrence R^{a} or a monovalent cationic group; or two R¹¹, together with the atoms to which they are attached, form a 4-8 membered cyclic phosphate group, or two R¹¹ together represent a divalent cationic group;
ring A is a C₆₋₁₀aryl or a 5-10 membered heteroaryl;
each R² is independently for each occurrence H, R^{e}, R^{b}, R^{e} substituted with one or more of the same or different R^{a} and/or R^{b}, —OR^{e} substituted with one or more of the same or different R^{a} and/or R^{b}, —SR^{e} substituted with one or more of the same or different R^{a} and/or R^{b}, —C(O)R^{e} substituted with one or more of the same or different R^{a} and/or R^{b}, —N(R^{a})R^{e} where R^{e} is substituted with one or more of the same or different R^{a} and/or R^{b}, —S(O)₂R^{e} substituted with one or more of the same or different R^{a} and/or R^{b}, —N(R^{a})—S(O)₂R^{e} where R^{e} is substituted with one or more of the same or different R^{a} and/or R^{b}, —B(OR^{a})₂, —B(N(R^{c})₂)₂, —(C(R^{a})₂)ₘ—R^{b}, —O—(C(R^{a})₂)ₘ—R^{b}, —S—(C(R^{a})₂)ₘ—R^{b}, —O—(C(R^{b})₂)ₘ—R^{a}, —N(R^{a})—(C(R^{a})₂)ₘ—R^{b}, —O—(CH₂)ₘ—CH((CH₂)ₘR^{b})R^{b}, —C(O)N(R^{a})—(C(R^{a})₂)ₘ—R^{b}, —O—(C(R^{a})₂)ₘ—C(O)N(R^{a})—(C(R^{a})₂)ₘ—Ŕ^{b}, —N((C(R^{a})₂)ₘR^{b})₂, —S—(C(R^{a})₂)ₘ—C(O)N(R^{a})—(C(R^{a})₂)ₘ—R^{b}, —N(R^{a})—C(O)—N(R^{a})—C(R^{a})₂)ₘ—R^{b}, —N(R^{a})—C(O)—(C(R^{a})₂)ₘ—C(R^{a})(R^{b})₂ or —N(R^{a})—(C(R^{a})₂)ₘ—C(O)—N(R^{a})—(C(R^{a})₂)ₘ—R^{b};
each R^{a} is independently for each occurrence H, deuterium, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-10 membered heteroalicyclyl, 4-11 membered heteroalicyclylalkyl, 5-15 membered heteroaryl or 6-16 membered heteroarylalkyl;
each R^{b} is independently for each occurrence ═O, —OR^{a}, —O—(C(R^{a})₂)ₘ—OR^{a}, haloC₁₋₃alkyloxy, ═S, ═NR^{a}, ═NOR^{a}, —N(R^{c})₂, halo, —CF₃, —CN, —NC, —OCN, —SCN, —NO, —NO₂, ═N₂, —N₃, —S(O)R^{a}, —S(O)₂R^{a}, —SO₃R^{a}, —S(O)N(R^{c})₂, —S(O)₂N(R^{c})₂, —OS(O)R^{a}, —OS(O)₂R^{a}, —OSO₃R^{a}, —OS(O)₂N(R^{c})₂, —C(O)R^{a}, —CO₂R^{a}, —C(O)N(R^{c})₂, —C(NR^{a})—N(R^{c})₂, —C(NOH)—R^{a}, —C(NOH)—N(R^{c})₂, —OC(O)R^{a}, —OC(O)OR^{a}, —OC(O)N(R^{c})₂, —OC(NH)—N(R^{c})₂, —OC(NR^{a})—N(R^{c})₂, —N(R^{a})—S(O)₂H, —[N(R^{a})C(O)]ₙR^{a}, —[N(R^{a})C(O)]ₙOR^{a}, —[N(R^{a})C(O)]ₙN(R^{c})₂ or —[N(R^{a})C(NR^{a})]ₙ—N(R^{c})₂;
each R^{c} is independently for each occurrence R^{a}, or, alternatively, two R^{c} are taken together with the nitrogen atom to which they are bonded to form a 3 to 10-membered heteroalicyclyl or a 5-10 membered heteroaryl which may optionally include one or more of the same or different additional heteroatoms and which is optionally substituted with one or more of the same or different R^{a} and/or R^{d} groups;
each R^{d} is ═O, —OR^{a}, haloC₁₋₃alkyloxy, C₁₋₆alkyl, ═S, —SR^{a}, ═NR^{a}, ═NOR^{a}, —N(R^{a})₂, halo, —CF₃, —CN, —NC, —OCN, —SCN, —NO, —NO₂, ═N₂, —N₃, —S(O)R^{a}, —S(O₂)R^{a}, —SO₃R^{a}, —S(O)N(R^{a})₂, —S(O)₂N(R^{a})₂, —OS(O)R^{a}, —OS(O)₂R^{a}, —OSO₃R^{a}, —OS(O)₂N(R^{a})₂, —C(O)R^{a}, —CO₂R^{a}, —C(O)N(R^{a})₂, —C(NR^{a})N(R^{a})₂, —C(NOH)R^{a}, —C(NOH)N(R^{a})₂, —OCO₂R^{a}, —OC(O)N(R^{a})₂, —OC(NR^{a})N(R^{a})₂, —[N(R^{a})C(O)]ₙR^{a}, —(C(R^{a})₂)ₙ—OR^{a}, —N(R^{a})—S(O)₂R^{a}, —C(O)—C₁₋₆haloalkyl, —S(O)₂C₁₋₆haloalkyl, —OC(O)R^{a}, —(C(R^{a})₂)ₘ—OR^{a}, —S(C(R^{a})₂)ₘ—OR^{a}, —N(R^{a})C₁₋₆haloalkyl, —P(O)(OR^{a})₂, —N(R^{a})—(C(R^{a})₂)ₘ—OR^{a}, —[N(R^{a})C(O)]ₙOR^{a}, —[N(R^{a})C(O)]ₙN(R^{a})₂, —[N(R^{a})C(NR^{a})]ₙN(R^{a})₂ or —N(R^{a})C(O)C₁₋₆haloalkyl; or two R^{d}, taken together with the atom or atoms to which they are attached, combine to form a 3-10 membered partially or fully saturated mono or bicyclic ring, optionally containing one or more heteroatoms and optionally substituted with one or more R^{a};
each R^{e} is independently for each occurrence C₁₋₆alkyl, C₃₋₈cycloalkyl, C₄₋₁₁ cycloalkylalkyl, C₆₋₁₀aryl, C₇₋₁₆arylalkyl, 2-6 membered heteroalkyl, 3-10 membered heteroalicyclyl, 4-11 membered heteroalicyclylalkyl, 5-15 membered heteroaryl or 6-16 membered heteroarylalkyl;
p is 0, 1, 2, 3 or 4;
each m is 1, 2 or 3;
each n is 0, 1, 2 or 3;
or two R² groups, taken together with the atom or atoms to which they are attached, combine to form a 4-10 membered partially or fully saturated mono or bicyclic ring, optionally containing one or more heteroatoms and optionally substituted with one or more R^{a} and/or R^{b};
Z¹ and Z² are each independently CH, CR² or N;
R³ is H, optionally substituted C₁₋₆alkyl or R⁵⁰;
R⁴ is H, optionally substituted C₁₋₆alkyl or R⁵⁰;
R⁵ is halo, —CN, optionally substituted C₁₋₆alkyl, alkynyl, hydroxy, optionally substituted C₁₋₆alkoxy, nitro, —N(R^{a})₂, —C(O)N(R^{a})₂, —CO₂R^{a} or —C(O)R^{a}; 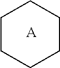
is selected from the group consisting of a six-membered aryl and a six-membered heteroaryl;
n′ is 0, 1 or 2;
m′ is 0, 1, 2, 3 or 4;
R¹² is selected from hydrogen, alkyl, alkenyl, alkynyl, haloalkyl, haloalkenyl, haloalkenyl, optionally substituted cycloalkyl, optionally substituted cycloalkylalkyl, optionally substituted cycloalkylalkenyl, optionally substituted cycloalkylalkynyl, optionally substituted aryl, optionally substituted aralkyl, optionally substituted arylalkenyl, optionally substituted aralkynyl, —R¹⁹—C(O)OR¹⁷, —R²⁰—N(R¹⁷)R¹⁸ and —R²⁰—OR¹⁷;
each R¹³, when present, is independently selected from the group consisting of optionally substituted alkyl, halo, haloalkyl, cyano, nitro, —OR¹⁷, —N(R¹⁷)₂, —C(O)OR¹⁷ and —C(O)N(R¹⁷)₂,
each R¹⁴, when present, is independently selected from the group consisting of alkyl, halo and haloalkyl;
R¹⁵ is selected from the group consisting of aryl and heteroaryl, where the aryl and the heteroaryl are each independently optionally substituted by one or more substituents selected from the group consisting of oxo, alkyl, halo, haloalkyl, cyano, N-heterocyclyl, N-heteroaryl, aryl, —R¹⁹—OR²¹, —R¹⁹—S(O)_{p′}R²¹ (where p′ is 0, 1 or 2), —R¹⁹—C(O)R²¹, —R¹⁹—C(O)OR²¹, —R¹⁹—C(O)N(R²¹)R²², —R¹⁹—N(R²¹)R²², —R¹⁹—N(R²¹)—R²⁰—N(R²¹)R²², —R¹⁹—N(R²¹)—R²⁰—OR²², —R¹⁹—N(R²¹)C(O)R²², —R¹⁹—N(R²¹)S(O)₂R²², —R¹⁹—N(R²¹)C(O)—R¹⁹—N(R²¹)R²², and —R¹⁹—N(R²¹)—R²⁰—N(R²¹)S(O)₂R²², wherein each R²² and R²¹ independently is selected from the group consisting of hydrogen, alkyl, optionally substituted cycloalkyl, optionally substituted heterocyclyl, optionally substituted heterocyclylalkyl, optionally substituted heteroaryl and optionally substituted aryalkyl, and where the N-heterocyclyl, the N-heteroaryl and the aryl are each independently optionally substituted by one or more substituents selected from the group consisting of —C(O)R¹⁷, —R¹⁹—N(R¹⁷)R¹⁸, —R¹⁹—C(O)N(R¹⁷)R¹⁸, alkyl, halo and optionally substituted aryl,
R¹⁶ is an N-heterocyclyl, wherein a nitrogen atom in the N-heterocyclyl is optionally substituted by a substituent selected from the group consisting of alkyl, alkenyl, alkynyl, haloalkyl, haloalkenyl, haloalkynyl, optionally substituted aryl, optionally substituted aralkyl, optionally substituted arylalkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁹—OR¹⁴, —R¹⁹—C(O)R¹⁷, —R¹⁹—C(O)OR¹⁷, —R¹⁹—N(R¹⁷)R¹⁸, —R¹⁹—C(O)N(R¹⁷)R¹⁸, —R¹⁹—C(N═R¹⁷)N(R¹⁷)R¹⁸, —R¹⁹—S(O)₂N(R¹⁷)R¹⁸, and —R¹⁹—S(O)ₜR¹⁷ (where t is 1 or 2); and a carbon atom in the N-heterocyclyl is optionally substituted by a substituent selected from the group consisting of alkyl, alkenyl, alkynyl, halo, haloalkyl, haloalkenyl, haloalkynyl, oxo, optionally substituted aryl, optionally substituted aralkyl, optionally substituted arylalkenyl, optionally substituted aralkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁹—OR¹⁷, —R¹⁹—C(O)R¹⁷, —R¹⁹—C(O)OR¹⁷, —R¹⁹—N(R¹⁷)R¹⁸, —R¹⁹—C(O)N(R¹⁷)R¹⁸, —R¹⁹—S(O)₂N(R¹⁷)R¹⁸, and —R¹⁹—S(O)_{p′}R¹⁷ (where p′ is 0, 1 or 2),
each R¹⁷ and each R¹⁸ is independently selected from the group consisting of hydrogen, alkyl, alkenyl, alkynyl, haloalkyl, haloalkenyl, haloalkynyl, optionally substituted cycloalkyl, optionally substituted cycloalkylalkyl, optionally substituted aryl, optionally substituted aralkyl, optionally substituted arylalkenyl, optionally substituted aralkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, and optionally substituted heteroarylalkynyl; or any R¹⁷ and R¹⁸, together with the common nitrogen to which they are both attached, form an optionally substituted N-heteroaryl or an optionally substituted N-heterocyclyl;
each R¹⁹ is independently selected from the group consisting of a direct bond, an optionally substituted straight or branched alkylene chain, an optionally substituted straight or branched alkenylene chain and an optionally substituted straight or branched alkynylene chain;
each R²⁰ is independently selected from the group consisting of an optionally substituted straight or branched alkylene chain, an optionally substituted straight or branched alkenylene chain and an optionally substituted straight or branched alkynylene chain; provided at least one of R¹⁶ and a substituent on R¹⁵ is a bridged N-heterocyclyl;
A¹ and A² are independently selected from C and N;
T, U, and V are independently selected from O, S, N, CR²⁷, and NR²⁸;
the 5-membered ring formed by A¹, A², U, T, and V is aromatic;
X¹ is N or CR²⁶;
Y¹ is C₁₋₈ alkylene, C₂₋₈ alkenylene, C₂₋₈ alkynylene, (CR³³R³⁴)_{p″}, —(C₃₋₁₀cycloalkylene)-(CR³³R³⁴), (CR³³R³⁴)_{p″}-(arylene)-(CR³³R³⁴)_{q}, (CR³³R³⁴)_{p″}—(C₁-C₁₀heterocycloalkylene)-(CR³³R³⁴)_{q}, (CR³³R³⁴)_{p″}-(heteroarylene)-(CR³³R³⁴)_{q}, (CR³³R³⁴)_{p″}O(CR³³R³⁴), (CR³³R³⁴)_{p″}S(CR³³R³⁴), (CR³³R³⁴)_{p″}C(O)(CR³³R³⁴), (CR³³R³⁴)_{p″}C(O)NR⁴¹(CR³³R³⁴)_{q}, (CR³³R³⁴)_{p″}C(O)O(CR³³R³⁴)_{q}, (CR³³R³⁴)_{p″}OC(O)(CCR³³R³⁴)_{q}, (CR³³R³⁴)_{p″}OC(O)NR⁴¹(CR³³R³⁴)_{q}, (CR³³R³⁴)_{q}, (CR³³R³⁴)ₚNR⁴¹(CR³³R³⁴)_{q}, (CCR³³R³⁴)ₚNR⁴¹C(O)NR⁴²(CR³³R³⁴)_{q}, (CR³³R³⁴)_{p″}S(O)(CR³³R³⁴)_{q}, (CR³³R³⁴)_{p″}S(O)NR⁴¹(CR³³R³⁴)_{q}, (CR³³R³⁴)_{p″}S(O)₂(CR³³R³⁴)_{q}, or (CR³³R³⁴)_{p″}S(O)₂NR⁴¹(CR³³R³⁴)_{q}, wherein the C₁₋₈ alkylene, C₂₋₈ alkenylene, C₂₋₈ alkynylene, cycloalkylene, arylene, heterocycloalkylene, or heteroarylene, is optionally substituted with 1, 2, or 3 substituents independently selected from -D¹-D²-D³-D⁴;
Z³ is H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, ═C—R⁴⁹, ═N—R⁴⁹, Cy¹, CN, NO₂, OR³⁵SR³⁵, C(O)R³⁶, C(O)NR⁴¹R⁴², C(O)OR³⁵, OC(O)R³⁶, OC(O)NR⁴¹R⁴², NR⁴¹R⁴², NR⁴¹C(O)R³⁶, NR⁴¹C(O)NR⁴¹R⁴², NR⁴¹C(O)OR³⁵, C(═NR⁴⁹)NR⁴¹R⁴², NR⁴¹C(═NR⁴⁹)NR⁴¹R⁴², S(O)R³⁶, S(O)NR⁴¹R⁴², S(O)₂R³⁶, NR⁴¹S(O)₂R³⁶, C(═NOH)R³⁶, C(═NO(C₁₋₆alkyl)R³⁶, and S(O)₂NR⁴¹R⁴², wherein when Z is H, n″ is 1, or the —(Y)_{n″}—Z moiety is taken together with i) A² to which the moiety is attached, ii) R²⁷ or R²⁸ of either T or V, and iii) the C or N atom to which the R²⁷ or R²⁸ of either T or V is attached to form a 4- to 20-membered aryl, cycloalkyl, heteroaryl, or heterocycloalkyl ring fused to the 5-membered ring formed by A¹, A², U, T, and V, wherein the 4- to 20-membered aryl, cycloalkyl, heteroaryl, or heterocycloalkyl ring is optionally substituted by 1, 2, 3, 4, or 5 substituents independently selected from —(W)_{1n″}-Q;
W is C₁₋₈ alkylenyl, C₂₋₈ alkenylenyl, C₂₋₈ alkynylenyl, O, S, C(O), C(O)NR⁴³, C(O)O, OC(O), OC(O)NR⁴³, NR⁴³, NR⁴³C(O)NR⁴⁴, S(O), S(O)NR⁴³, S(O)₂, or S(O)₂NR⁴³;
Q is H, halo, CN, NO₂, C₁₋₈ alkyl, C₂₋₈ alkenyl, C₂₋₈ alkynyl, C₁₋₈ haloalkyl, halosulfanyl, aryl, cycloalkyl, heteroaryl, or heterocycloalkyl, wherein the C₁₋₈ alkyl, C₂₋₈ alkenyl, C₂₋₈ alkynyl, C₁₋₈ haloalkyl, aryl, cycloalkyl, heteroaryl, or heterocycloalkyl is optionally substituted with 1, 2, 3 or 4 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy², CN, NO₂, OR³⁷, SR³⁷, C(O)R³⁸, C(O)NR⁴³R⁴⁴, C(O)OR³⁷, OC(O)R³⁸, OC(O)NR⁴³R⁴⁴, NR⁴³R⁴⁴, NR⁴³C(O)R³⁸, NR⁴³C(O)NR⁴³R⁴⁴, NR⁴³C(O)OR³⁷, S(O)R³⁸, S(O)NR⁴³R⁴⁴, S(O)₂R³⁸, NR⁴³S(O)₂R³⁸ and S(O)₂NR⁴³R⁴⁴;
Cy¹ and Cy² are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, CN, NO₂, —OR³⁹, SR³⁹, C(O)R⁴⁰, C(O)R⁴⁵R⁴⁶, C(O)OR³⁹, OC(O)R⁴⁰, OC(O)NR⁴⁵R⁴⁶, NR⁴⁵R⁴⁶, NR⁴⁵C(O)R⁴⁰, NR⁴⁵C(O)OR³⁹, NR⁴⁵S(O)R⁴⁰, NR⁴⁵S(O)₂R⁴⁰, S(O)R⁴⁰, S(O)NR⁴⁵R⁴⁶, S(O)₂R⁴⁰, and S(O)₂NR⁴⁵R⁴⁶;
R²³, R²⁴, R²⁵, and R²⁶ are independently selected from H, halo, C₁₋₄ alkyl, C₂₋₄ alkenyl, C₂₋₄ alkynyl, C₁₋₄ haloalkyl, halosulfanyl, aryl, cycloalkyl, heteroaryl, heterocycloalkyl, CN, NO₂, OR²⁹, SR²⁹, C(O)R³⁰, C(O)NR³¹R³², C(O)OR²⁹OC(O)R³⁰, OC(O)NR³¹R³², NR³¹R³², NR³¹C(O)R³⁰, NR³²C(O)OR²⁹, S(O)R³⁰, S(O)NR³¹R³², S(O)₂R³⁰, NR³¹S(O)₂R³⁰, and S(O)₂NR³¹R³²;
R²⁷ is H, halo, C₁₋₄ alkyl, C₂₋₄ alkenyl, C₂₋₄ alkynyl, C₁₋₄ haloalkyl, halosulfanyl, CN, NO₂, OR²⁹, SR²⁹, C(O)R³⁰, C(O)NR³¹R³², C(O)OR²⁹, OC(O)R³⁰, OC(O)NR³¹R³², NR³¹R³², NR³¹C(O)R³⁰, NR³¹C(O)OR²⁹, S(O)R³⁰, S(O)NR³¹R³², S(O)₂R³⁰, NR³¹S(O)₂R³⁰, or S(O)₂NR³¹R³²;
R²⁸ is H, C₁₋₄ alkyl, C₂₋₄ alkenyl, C₂₋₄ alkynyl, C₁₋₄ haloalkyl, OR²⁹, C(O)R³⁰, C(O)NR³¹R³², C(O)OR²⁹, S(O)R³⁰, S(O)NR³¹R³², S(O)₂R³⁰, or S(O)₂NR³¹R³²;
R²⁹ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;
R³⁰ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;
R³¹ and R³² are independently selected from H, C₁₋₁₀ alkyl, C₁₋₆ haloalkyl, C₂₋₆ alkenyl, C₂₋₆ alkynyl, C₁₋₆ alkylcarbonyl, arylcarbonyl, C₁₋₆ alkylsulfonyl, arylsulfonyl, aryl, heteroaryl, cycloalkyl, heterocycloalkyl, arylalkyl, heteroarylalkyl, cycloalkylalkyl and heterocycloalkylalkyl;
or R³¹ and R³² together with the N atom to which they are attached form a 4-, 5-, 6- or 7-membered heterocycloalkyl group;
R³³ and R³⁴ are independently selected from H and -E¹-E²-E³-E⁴;
D¹ and E¹ are independently absent or independently selected from absent or selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆alkyl, C₁₋₆ haloalkyl, C₂₋₈ alkoxyalkyl, C₁₋₆ alkoxy, C₁₋₆haloalkoxy, amino, C₁₋₆ alkylamino, and C₂₋₈ dialkylamino;
D² and E² are independently absent or independently selected from C₁₋₆ alkylene, C₂₋₆ alkenylene, C₂₋₆ alkynylene, (C₁₋₆alkylene)ᵣ-O—(C₁₋₆alkylene)ₛ, (C₁₋₆alkylene)ᵣ-S—(C₁₋₆alkylene)ₛ, (C₁₋₆ alkylene)ᵣ-NR⁴⁷—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR⁴⁷—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆alkylene)ₛ, (C₁₋₆ alkylene)ᵣ-SONR⁴¹—(C₁₋₆alkylene)ₛ, and (C₁₋₆alkylene)ᵣ-NR⁴⁷CONR⁴⁸—(C₁₋₆ alkylene)ₛ, wherein each of the C₁₋₆ alkylene, C₂₋₆ alkenylene, and C₂₋₆ alkynylene is optionally substituted by 1, 2 or 3 substituents independently selected from halo, CN, NO₂, N₃, SCN, OH, C₁₋₆ alkyl, C₁₋₆ haloalkyl, C₂₋₈ alkoxyalkyl, Ĉalkoxy, C₁₋₆ haloalkoxy, amino, C₁₋₆ alkylamino, and C₂₋₈ dialkylamino;
D³ and E³ are independently absent or independently selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆ alkyl, C₁₋₆haloalkyl, C₂₋₈ alkoxyalkyl, C₁₋₆ alkoxy, C₁₋₆ haloalkoxy, amino, C₁₋₆ alkylamino, and C₂₋₈ dialkylamino;
D⁴ and E⁴ are independently selected from H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, CN, NO₂, OR³⁵, SR³⁵, C(O)R³⁶, C(O)NR⁴¹R⁴², C(O)OR³⁵, OC(O)R³⁶, OC(O)NR⁴¹R⁴², NR⁴¹R⁴², NR⁴¹C(O)R³⁶, NR⁴¹C(O)NR⁴¹R⁴², NR⁴¹C(O)OR³⁵, C(═NR⁴⁹)NR⁴¹R⁴², NR⁴¹C(═NR⁴⁹)NR⁴¹R⁴², S(O)R³⁶, S(O)NR⁴¹R⁴², S(O)₂R³⁶, NR⁴¹S(O)₂R³⁶, C(═NOH)R³⁶, C(═NO(C₁₋₆ alkyl)R³⁶, and S(O)₂NR⁴¹R⁴², wherein the C₁₋₈ alkyl, C₂₋₈ alkenyl, or C₂₋₈ alkynyl, is optionally substituted with 1, 2, 3, 4, 5, or 6 substituents independently selected from halo, C₁₋₄ alkyl, C₂₋₄ alkenyl, C₂₋₄ alkynyl, C₁₋₄haloalkyl, halosulfanyl, C₁₋₄hydroxyalkyl, C₁₋₄cyanoalkyl, Cy¹, CN, NO₂, OR³⁵, SR³⁵, C(O)R³⁶. C(O)NR⁴¹R⁴², C(O)OR³⁵, OC(O)R³⁶, OC(O)NR⁴¹R⁴², NR⁴¹R⁴², NR⁴¹C(O)R³⁶, NR⁴¹C(O)NR⁴¹R⁴², NR⁴¹C(O)OR³⁵, C(═NR⁴⁹)NR⁴¹R⁴², NR^{c}C(═NR⁴⁹)NR⁴¹R⁴², S(O)R³⁶, S(O)NR⁴¹R⁴², S(O)₂R²⁸, NR⁴¹S(O)₂R³⁶, C(═NOH)R³⁶, C(═NO(C₁₋₆alkyl))R³⁶, and S(O)₂NR⁴¹R⁴²;
R³⁵ is Cy¹, —(C₁₋₆alkyl)-Cy¹, C₁₋₆ alkyl, C₁₋₆ haloalkyl, C₂₋₆ alkenyl, C₂₋₆ alkynyl, wherein the C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
R³⁶ is H, Cy¹, alkyl)-Cy′, C₁₋₆ alkyl, C₁₋₆ haloalkyl, C₂₋₆ alkenyl, C₂₋₆ alkynyl, wherein the C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆alkyl, C₁₋₆ haloalkyl, C₁₋₆ haloalkyl, halosulfanyl, aryl, arylalkyl, heteroaryl, heteroarylalkyl, cycloalkyl and heterocycloalkyl;
R³⁷ and R³⁹ are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein the C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;
R³⁸ and R⁴⁰ are independently selected from H, C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein the C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆haloalkyl, C₁₋₆haloalkyl, halosulfanyl, aryl, arylalkyl, heteroaryl, heteroarylalkyl, cycloalkyl and heterocycloalkyl;
R⁴¹ and R⁴² are independently selected from H, Cy¹, —(C₁₋₆alkyl)-Cy¹, C₁₋₁₀ alkyl, C₁₋₆haloalkyl, C₂₋₆ alkenyl, C₂₋₆ alkynyl, wherein the C₁₋₁₀ alkyl, C₁₋₆ haloalkyl, C₂₋₆ alkenyl, or C₂₋₆ alkynyl, is optionally substituted with 1, 2, or 3 substituents independently selected from Cy¹, —(C₁₋₆alkyl)-Cy′, OH, CN, amino, halo, C₁₋₆alkyl, C₁₋₆haloalkyl, C₁₋₆haloalkyl, and halosulfanyl; or
R⁴¹ and R⁴² together with the N atom to which they are attached form a 4-, 5-, 6- or 7-membered heterocycloalkyl group optionally substituted with 1, 2, or 3 substituents independently selected from Cy¹, —(C₁₋₆ alkyl)-Cy¹, OH, CN, amino, halo, C₁₋₆alkyl, C₁₋₆haloalkyl, C₁₋₆haloalkyl, and halosulfanyl;
R⁴³ and R⁴⁴ are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein the C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl; or
R⁴³ and R⁴⁴ together with the N atom to which they are attached form a 4-, 5-, 6- or 7-membered heterocycloalkyl group optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;
R⁴⁵ and R⁴⁶ are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein the C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl; or
R⁴⁵ and R⁴⁶ together with the N atom to which they are attached form a 4-, 5-, 6- or 7-membered heterocycloalkyl group optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;
R⁴⁷ and R⁴⁸ are independently selected from H and C₁₋₆alkyl;
R⁴⁹ is H, CN, C₁₋₆ alkyl or NO₂;
m″ is 0 or 1;
n″ is 0 or 1;
p″ is 0, 1, 2, 3, 4, 5, or 6;
q is 0, 1, 2, 3, 4, 5, or 6;
r is 0 or 1;
s is 0 or 1;
R⁵² is a group of the formula 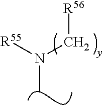
wherein y is 0, 1 or 2 or heteroaryl;
R⁵⁵ is selected from the group consisting of hydrogen, (C₁-C₆)alkyl, (C₁-C₆)alkylsulfonyl, (C₂-C₆)alkenyl, (C₂-C₆)alkynyl wherein the alkyl, alkenyl and alkynyl groups are optionally substituted by deuterium, hydroxy, amino, trifluoromethyl, (C₁-C₄)alkoxy, (C₁-C₆)acyloxy, (C₁-C₆)alkylamino, ((C₁-C₆)alkyl)₂amino, cyano, nitro, (C₂-C₆)alkenyl, (C₂-C₆)alkynyl or (C₁-C₆)acylamino, or R⁵⁵ is (C₃-C₁₀)cycloalkyl wherein the cycloalkyl group is optionally substituted by deuterium, hydroxy, amino, trifluoromethyl, (C₁-C₆)acyloxy, (C₁-C₆)acylamino, (C₁-C₆)alkylamino, ((C₁-C₆)alkyl)₂amino, cyano, cyano(C₁-C₆)alkyl, trifluoromethyl(C₁-C₆)alkyl, nitro, nitro(CC₆)alkyl or (C₁-C₆)acylamino;
R⁵⁶ is (C₂-C₉)heterocycloalkyl wherein the heterocycloalkyl groups must be substituted by one to five carboxy, cyano, amino, deuterium, hydroxy, (C₁-C₆)alkyl, (C₁-C₆)alkoxy, halo, (C₁-C₆)acyl, (C₁-C₆alkylamino), amino(C₁-C₆)alkyl, (C₁-C₆)alkoxy-CO—NH, (C₁-C₆)alkylamino-CO—, (C₂-C₆)alkenyl, (C₂-C₆)alkynyl, (C₁-C₆)alkylamino, amino(C₁-C₆)alkyl, hydroxy(C₁-C₆)alkyl, (C₁-C₆)alkoxy(C₁-C₆)alkyl, (C₁-C₆)acyloxy(C₁-C₆)alkyl, nitro, cyano(C₁-C₆)alkyl, halo(C₁-C₆)alkyl, nitro(C₁-C₆)alkyl, trifluoromethyl, trifluoromethyl(C₁-C₆)acyl, (C₁-C₆)acylamino, (C₁-C₆)acylamino(C₁-C₆)alkyl, (C₁-C₆)alkoxy(C₁-C₆)acylamino, amino(C₁-C₆)acyl, amino(C₁-C₆)acyl(C₁-C₆)alkyl, (C₁-C₆)alkylamino(C₁-C₆)acyl, ((C₁-C₆)alkyl)₂amino(C₁-C₆)acyl, R⁶⁴R⁶⁵N—CO—O—, R⁶⁴R⁶⁵N—CO—(C₁-C₆)alkyl, (C₁-C₆)alkyl-S(O)_{m′″}, R⁶⁴R⁶⁵NS(O)_{m′″}, R⁶⁴R⁶⁵NS(O)_{m′″}(C₁-C₆)alkyl, R⁶⁴S(O)_{m′″}R⁶⁵N, R⁶⁴S(O)_{m′″}—R⁶⁵N(C₁-C₆)alkyl wherein m is 0, 1 or 2 and R⁶⁴ and R⁶⁵ are each independently selected from hydrogen or a group of the formula 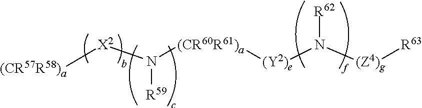
wherein a is 0, 1, 2, 3 or 4;
b, c, e, f and g are each independently 0 or 1, d is 0, 1, 2, or 3;
X² is S(O)_{n′″} wherein n is 0, 1 or 2, oxygen, carbonyl or —C(═N-cyano)-;
Y² is S(O)_{n′″} wherein n is 0, 1 or 2; or carbonyl;
Z⁴ is carbonyl, C(O)O—, C(O)NR— or S(O)_{n′″} wherein n′″ is 0, 1 or 2;
R⁵⁷, R⁵⁸, R⁵⁹, R⁶⁰, R⁶¹ and R⁶² are each independently selected from the group consisting of hydrogen or (C₁-C₆)alkyl optionally substituted by deuterium, hydroxy, amino, trifluoromethyl, (C₁-C₆)acyloxy, (C₁-C₆)acylamino, ((C₁-C₆)alkyl)₂amino, cyano, cyano(C₁-C₆)alkyl, trifluoromethyl(C₁-C₆)alkyl, nitro, nitro(C₁-C₆)alkyl, or (C₁-C₆)acylamino,
R⁶³ is carboxy, cyano, amino, oxo, deuterium, hydroxy, trifluoromethyl, (C₁-C₆)alkyl, trifluoromethy C₁-C₆alkyl, (C₁-C₆)alkoxy, halo, (C₁-C₆)acyl, (C₁-C₆)alkylamino, ((C₁-C₆)alkyl)₂ amino, amino(C₁-C₆)alkyl, (C₁-C₆)alkoxy-CO—NH—, (C₁-C₆)alkylamino-CO—, (C₂-C₆)alkenyl, (C₂-C₆)alkynyl, (C₁-C₆)alkylamino, hydroxy(C₁-C₆)alkyl, (C₁-C₆)alkoxy(C₁-C₆)alkyl, (C₁-C₆)acyloxy(C₁-C₆)alkyl, nitro, cyano(C₁-C₆)alkyl, halo(C₁-C₆)alkyl, nitro(C₁-C₆)alkyl, trifluoromethyl, trifluoromethyl(C₁-C₆)alkyl, (C₁-C₆)acylamino, (C₁-C₆)acylamino(C₁-C₆)alkyl, (C₁-C₆)alkoxy(C₁-C₆)acylamino, amino(C₁-C₆)acyl, amino(C₁-C₆)acyl(C₁-C₆)alkyl, (C₁-C₆alkylamino(C₁-C₆)acyl, ((C₁-C₆)alkyl)₂amino(C₁-C₆)acyl, R⁶⁴R⁶⁵N—CO—O—, R⁶⁴R⁶⁵N—CO—(C₁-C₆)alkyl, R⁶⁴C(O)NH, R⁶⁴OC(O)NH, R⁶⁴NHC(O)NH, C₁-C₆)alkyl-S(O)_{m′″}, (C₁-C₆)alkyl-S(O)_{m′″}—(C₁-C₆)alkyl, R⁶⁴R⁶⁵NS(O)_{m′″}, R⁶⁴R⁶⁵NR(O)_{m′″}(C₁-C₆alkyl, R⁶⁴S(O)_{m′″}R⁶⁵N, R⁶⁴S(O)_{m′″}R⁶⁵N(C₁-C₆)alkyl wherein m is 0, 1 or 2 and R⁶⁴ and R⁶⁵ are each independently selected from hydrogen or (C₁-C₆)alkyl;
R⁵³ and R⁵⁴ are each independently selected from the group consisting of hydrogen, deuterium, amino, halo, hydroxy, nitro, carboxy, (C₂-C₆)alkenyl, (C₂-C₆)alkynyl, trifluoromethyl, trifluoromethoxy, (C₁-C₆)alkyl, (C₁-C₆alkoxy, (C₃-C₁₀)cycloalkyl wherein the alkyl, alkoxy or cycloalkyl groups are optionally substituted by one to three groups selected from halo, hydroxy, carboxy, amino (C₁-C₆)alkylthio, (C₁-C₆alkylamino, ((C₁-C₆)alkyl)₂amino, (C₅-C₉)heteroaryl, (C₂-C₉)heterocycloalkyl, (C₃-C₉)cycloalkyl or (C₆-C₁₀)aryl, or R⁵³ and R⁵⁴ are each independently (C₃-C₁₀)cycloalkyl, (C₃-C₁₀)cycloalkoxy, (C₁-C₆)alkylamino ((C₁-C₆)alkyl)₂amino, (C₆-C₁₀)arylamino, (C₁-C₆)alkylthio, (C₆-C₁₀)arylthio, (C₁-C₆)alkylsulfinyl, (C₆-C₁₀)arylsulfinyl, (C₁-C₆)alkylsulfonyl, (C₆-C₁₀)arylsulfonyl, (C₁-C₆)acyl, (C₁-C₆alkoxy-CO—NH—, (C₁-C₆)alkyamino-CO—, (C₅-C₉)heteroaryl, (C₂-C₉)heterocycloalkyl or (C₆-C₁₀)aryl wherein the heteroaryl, heterocycloalkyl and aryl groups are optionally substituted by one to three halo, (C₁-C₆)alkyl, (C₁-C₆)alkyl-CO—NH—, (C₁-C₆)alkyoxy-CO—NH—, (C₁-C₆)alkyl-CO—NH—(C₁-C₆)alkyl, (C₁-C₆)alkoxy-CO—NH—(C₁-C₆)alkyl, (C₁-C₆alkoxy-CO—NH—(C₁-C₆)alkoxy, carboxy, carboxy(C₁-C₆)alkyl, carboxy(C₁-C₆)alkoxy, benzyloxycarbonyl(C₁-C₆)alkoxy, C₆alkoxycarbonyl(C₁-C₆)alkoxy, (C₆-C₁₀)aryl, amino, amino(C₁-C₆)alkyl, (C₁-C₆)alkoxycarbonylamino, (C₆-C₁₀)aryl(C₁-C₆)alkoxycarbonylamino, (C₁-C₆)alkylamino, ((C₁-C₆)alkyl)₂amino, (C₁-C₆)alkylamino(C₁-C₆)alkyl, ((C₁-C₆)alkyl)₂amino(C₁-C₆)alkyl, hydroxy, (C₁-C₆alkoxy, carboxy, carboxy(C₁-C₆)alkyl, (C₁-C₆alkoxycarbonyl, (C₁-C₆alkoxycarbony C₁-C₆alkyl, (C₁-C₆)alkoxy-CO—NH—, (C₁-C₆)alkyl-CO—NH—, cyano, (C₅-C₉)heterocycloalkyl, amino-CO—NH—, (C₁-C₆)alkylamino-CO—NH—, ((C₁-C₆)alkyl)₂amino-CO—NH—, (C₆-C₁₀)arylamino-CO—NH—, (C₅-C₉)heteroarylamino-CO—NH—, (C₁-C₆)alkylamino-CO—NH—(C₁-C₆)alkyl, ((C₁-C₆)alkyl)₂amino-CO—NH—(C₁-C₆)alkyl, (C₆-C₁₀)arylamino-CO—NH—(C₁-C₆)alkyl, (C₅-C₉)heteroarylamino-CO—NH—(C₁-C₆)alkyl, (C₁-C₆)alkylsulfonyl, —C₆)alkylsulfonylamino, (C₁-C₆)alkylsulfonylamino(C₁-C₆)alkyl, (C₆-C₁₀)arylsulfonyl, (C₆-C₁₀)arylsulfonylamino, (C₆-C₁₀)arylsulfonylamino(C₁-C₆)alkyl, (C₁-C₆)alkylsulfonylamino, (C₁-C₆)alkylsulfonylamino(C₁-C₆)alkyl, (C₅-C₉)heteroaryl or (C₂-C₉)heterocycloalkyl;
wherein R⁶⁸ is C₁₋₃alkyl or cyclopropyl, R⁶⁹ is CH₂CH₃, CH₂CF₃, CH₂CH₂CF₃, 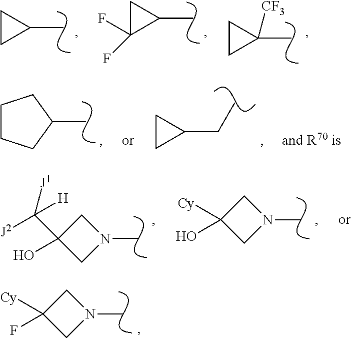
wherein J¹ is H or CH₃, J² is C₁₋₄alkyl, and Cy is C₃₋₅cycloalkyl;
R⁷¹ is T¹-R′ or is —Si(R′)₃;
R⁷², R⁷³, and R⁷⁴ are each independently halogen, CN, NO₂, or V¹—R;
X³, X⁴ and X⁵ are each independently N, or CH, wherein the hydrogen atom of CH is optionally replaced by R⁷⁵;
x is 1, 2, 3, or 4;
each occurrence of R⁷⁵ is independently halogen, CN, NO₂, or U¹—R′;
T¹, V¹, and U¹ are each independently a bond or an optionally substituted C₁-C₆ alkylidene chain, wherein up to two methylene units of the chain are optionally and independently replaced by —NR′—, —S—, —O—, —CS—, —CO₂—, —OCO—, —CO—, —COCO—, —CONR′—, —NR′CO—, —NR′CO₂—, —SO₂NR′—, —NR′SO₂—, —CONR′NR′—, —NR′CONR′—, —OCONR′—, —NRNR′—, —NR′SO₂NR′—, —SO—, —SO₂—, —PO—, —PO₂—, or —POR′—;
each occurrence of R′ is independently hydrogen or an optionally substituted group selected from a C₁-C₆ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or
two occurrences of R′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur; or
two occurrences of R′ that form a ring are on a single substituent and form a monocyclic or bicyclic; or
two occurrences of R′ are on two substituents and form a bicyclic fused ring with the ring to which the R⁷⁵ substituents are attached;
X⁶ is N or CR⁷⁹;
R⁷⁷ is H, F, R′, OH, OR′, COR′, COOH, COOR′, CONH₂, CONHR′, CON(R′)₂, or CN;
R⁷⁹ is H, F, R′, OH, OR′, COR′, COOH, COOR′, CONH₂, CONHR′, CON(R′)₂, or CN;
or R⁷⁷ and R⁷⁹, taken together, form a 5-7 membered aryl or heteroaryl ring optionally substituted with 1-4 occurrences of R⁸⁰;
R′ is a C₁₋₃aliphatic optionally substituted with 1-4 occurrences of R⁸¹;
each R⁸¹ is independently selected from halogen, CF₃, OCH₃, OH, SH, NO₂, NH₂, SCH₃, NCH₃, CN or unsubstituted C₁₋₂ aliphatic, or two R⁸¹ groups, together with the carbon to which they are attached, form a cyclopropyl ring or C═O;
each R⁸⁰ is independently selected from halogen, OCH₃, OH, NO₂, NH₂, SH, SCH₃, NCH₃, CN or unsubstituted C₁₋₂aliphatic;
R⁷⁶ is 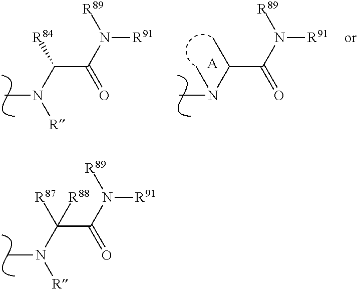
R″ is H or is a —C₁₋₂ aliphatic optionally substituted with 1-3 occurrences of R⁸²;
each R⁸² is independently selected from halogen, OCH₃, OH, SH, NO₂, NH₂, SCH₃, NCH₃, CN, CON(R⁸³)₂ or unsubstituted C₁₋₂ aliphatic, or two R⁸² groups, together with the carbon to which they are attached, form a cyclopropyl ring or C═O;
R⁸⁴ is a C₁₋₄ aliphatic optionally substituted with 1-5 occurrences of R⁸⁵;
each R⁸⁵ is independently selected from halogen, OCH₃, OH, NO₂, NH₂, SH, SCH₃, NCH₃, CN or unsubstituted C₁₋₂aliphatic, or two R⁸⁵ groups, together with the carbon to which they are attached, form a cyclopropyl ring;
ring A is a 4-8 membered saturated nitrogen-containing ring comprising up to two additional heteroatoms selected from N, O, or S and optionally substituted with 1-4 occurrences of R⁸⁶;
each R⁸⁶ is independently selected from halogen, R′, NH₂, NHR′, N(R)₂, SH, SR′, OH, OR′, NO₂, CN, CF₃, COOR′, COOH, COR′, OC(O)H, OC(O)R′, CONH₂, CONHR′, CON(R)₂, NHC(O)R′ or NR′C(O)R; or any two R⁸⁶ groups, on the same substituent or different substituents, together with the atom(s) to which each R⁸⁶ group is bound, form a 3-7 membered saturated, unsaturated, or partially saturated carbocyclic or heterocyclic ring optionally substituted with 1-3 occurrences of R⁸¹;
R⁸⁷ is C₁₋₄ aliphatic optionally substituted with 1-5 occurrences of R⁸⁵;
R⁸⁸ is C₁₋₂ alkyl; or
R⁸⁷ and R⁸⁸ are taken together to form a 3-7 membered carbocyclic or heterocyclic saturated ring optionally substituted with 1-5 occurrences of R⁸⁵;
R⁸⁹ is H or unsubstituted C₁₋₂ alkyl;
R⁹⁰ is H or unsubstituted C₁₋₂ alkyl; and
R⁹¹ is a C₂₋₃ aliphatic or cycloaliphatic optionally substituted with up to 6 occurrences of F.

9. The method according to claim 8 wherein the kinase inhibitor is a pyrimidine-containing compound having a formula I 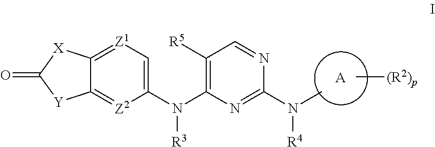
or a pharmaceutically acceptable salt thereof, wherein:
X and Y are each independently O, S, S(O), SO₂ or NR¹;
each R¹ is independently for each occurrence H, optionally substituted C₁₋₆alkyl, C(O)—C₁₋₆alkyl, CO₂—C₁₋₆alkyl or R⁵⁰;
each R⁵⁰ is —C(R⁹)₂-A-R¹⁰, where A is O or S;
each R⁹ is independently for each occurrence H, optionally substituted C₁₋₆alkyl, optionally substituted C₆₋₁₀aryl or optionally substituted C₇₋₁₆arylalkyl; or alternatively, two R⁹, together with the carbon to which they are attached, form an optionally substituted C₃₋₈cycloalkyl group or an optionally substituted 3-8 membered heteroalicyclyl;
R¹⁰ is R^{a} or —P(O)(OR¹¹)₂;
each R¹¹ is independently for each occurrence R^{a} or a monovalent cationic group; or two R¹¹, together with the atoms to which they are attached, form a 4-8 membered cyclic phosphate group, or two R¹¹ together represent a divalent cationic group;
ring A is a C₆₋₁₀aryl or a 5-10 membered heteroaryl;
each R² is independently for each occurrence H, R^{e}, R^{b}, R^{e} substituted with one or more of the same or different R^{a} and/or R^{b}, —OR^{e} substituted with one or more of the same or different R^{a} and/or R^{b}, —SR^{e} substituted with one or more of the same or different R^{a} and/or R^{b}, —C(O)R^{e} substituted with one or more of the same or different R^{a} and/or R^{b}, —N(R^{a})R^{e} where R^{e} is substituted with one or more of the same or different R^{a} and/or R^{b}, —S(O)₂R^{e} substituted with one or more of the same or different R^{a} and/or R^{b}, —N(R^{a})—S(O)₂R^{e} where R^{e} is substituted with one or more of the same or different R^{a} and/or R^{b}, —B(OR^{a})₂, —B(N(R^{c})₂)₂, —(C(R^{a})₂)ₘ—R^{b}, —O—(C(R^{a})₂)ₘ—R^{b}, —S—(C(R^{a})₂)ₘ—R^{b}, —O—(C(R^{b})₂)ₘ—R^{a}, —N(R^{a})—(C(R^{a})₂)ₘ—R^{b}, —O—(CH₂)ₘ—CH((CH₂)ₘR^{b})R^{b}, —C(O)N(R^{a})—(C(R^{a})₂)ₘ—R^{b}, —O—(C(R^{a})₂)ₘ—C(O)N(R^{a})—(C(R^{a})₂)ₘ—R^{b}, —N((C(R^{a})₂)ₘR^{b})₂, —S—(C(R^{a})₂)ₘ—C(O)N(R^{a})—(C(R^{a})₂)ₘ—R^{b}, —N(R^{a})—C(O)—N(R^{a})—(C(R^{a})₂)ₘ—R^{b}, —N(R^{a})—C(O)—(C(R^{a})₂)ₘ—C(R^{a})(R^{b})₂ or —N(R^{a})—(C(R^{a})₂)ₘ—C(O)—N(R^{a})—(C(R^{a})₂)ₘ—R^{b};
each R^{a} is independently for each occurrence H, deuterium, C₁₋₆alkyl, C₃₋₈cycloalkyl, C₄₋₁₁cycloalkylalkyl, C₆₋₁₀aryl, C₇₋₁₆arylalkyl, 2-6 membered heteroalkyl, 3-10 membered heteroalicyclyl, 4-11 membered heteroalicyclylalkyl, 5-15 membered heteroaryl or 6-16 membered heteroarylalkyl;
each R^{b} is independently for each occurrence ═O, —OR^{a}, —O—(C(R^{a})₂)ₘ—OR^{a}, haloC₁₋₃alkyloxy, ═S, —SR^{a}, ═NR^{a}, ═NOR^{a}, —N(R^{c})₂, halo, —CF₃, —CN, —NC, —OCN, —SCN, —NO, —NO₂, ═N₂, —N₃, —S(O)R^{a}, —S(O)₂R^{a}, —SO₃R^{a}, —S(O)N(R^{c})₂, —S(O)₂N(R^{c})₂, —OS(O)R^{a}, —OS(O)₂R^{a}, —OSO₃R^{a}, —OS(O)₂N(R^{c})₂, —C(O)R^{a}, —CO₂R^{a}, —C(O)N(R^{c})₂, —C(NR^{a})—N(R^{c})₂, —C(NOH)—R^{a}, —C(NOH)—N(R^{c})₂, —OC(O)R^{a}, —OC(O)OR^{a}, —OC(O)N(R^{c})₂, —OC(NH)—N(R^{c})₂, —OC(NR^{a})—N(R^{c})₂, —N(R^{a})—S(O)₂H, —[N(R^{a})C(O)]ₙR^{a}, —[N(R^{a})C(O)]ₙOR^{a}, —[N(R^{a})C(O)]ₙN(R^{c})₂ or —[N(R^{a})C(NR^{a})]ₙ—N(R^{c})₂;
each R^{c} is independently for each occurrence R^{a}, or, alternatively, two R^{c} are taken together with the nitrogen atom to which they are bonded to form a 3 to 10-membered heteroalicyclyl or a 5-10 membered heteroaryl which may optionally include one or more of the same or different additional heteroatoms and which is optionally substituted with one or more of the same or different R^{a} and/or R^{d} groups;
each R^{d} is ═O, —OR^{a}, haloC₁₋₃alkyloxy, C₁₋₆alkyl, ═S, —SR^{a}, ═NR^{a}, ═NOR^{a}, —N(R^{a})₂, halo, —CF₃, —CN, —NC, —OCN, —SCN, —NO, —NO₂, ═N₂, —N₃, —S(O)R^{a}, —S(O₂)R^{a}, —SO₃R^{a}, —S(O)N(R^{a})₂, —S(O)₂N(R^{a})₂, —OS(O)R^{a}, —OS(O)₂R^{a}, —OSO₃R^{a}, —OS(O)₂N(R^{a})₂, —C(O)R^{a}, —CO₂R^{a}, —C(O)N(R^{a})₂, —C(NR^{a})N(R^{a})₂, —C(NOH)R^{a}, —C(NOH)N(R^{a})₂, —OCO₂R^{a}, —OC(O)N(R^{a})₂, —OC(NR^{a})N(R^{a})₂, —[N(R^{a})C(O)]ₙR^{a}, —(C(R^{a})₂)ₙ—OR^{a}, —N(R^{a})—S(O)₂R^{a}, —C(O)—C₁₋₆haloalkyl, —S(O)₂C₁₋₆haloalkyl, —OC(O)R^{a}, —O(C(R^{a})₂)ₘ—OR^{a}, —S(C(R^{a})₂)ₘ—OR^{a}, —N(R^{a})C₁₋₆haloalkyl, —P(O)(OR^{a})₂, —N(R^{a})—(C(R^{a})₂)ₘ—OR^{a}, —[N(R^{a})C(O)]ₙOR^{a}, —[N(R^{a})C(O)]ₙN(R^{a})₂, —[N(R^{a})C(NR^{a})]ₙN(R^{a})₂ or —N(R^{a})C(O)C₁₋₆haloalkyl; two R^{d}, taken together with the atom or atoms to which they are attached, combine to form a 3-10 membered partially or fully saturated mono or bicyclic ring, optionally containing one or more heteroatoms and optionally substituted with one or more R^{a};
each R^{e} is independently for each occurrence C₁₋₆alkyl, C₃₋₈cycloalkyl, C₄₋₁₁ cycloalkylalkyl, C₆₋₁₀aryl, C₇₋₁₆arylalkyl, 2-6 membered heteroalkyl, 3-10 membered heteroalicyclyl, 4-11 membered heteroalicyclylalkyl, 5-15 membered heteroaryl or 6-16 membered heteroarylalkyl;
p is 0, 1, 2, 3 or 4;
each m independently is 1, 2 or 3;
each n independently is 0, 1, 2 or 3;
two R² groups, taken together with the atom or atoms to which they are attached, combine to form a 4-10 membered partially or fully saturated mono or bicyclic ring, optionally containing one or more heteroatoms and optionally substituted with one or more R^{a} and/or R^{b};
Z¹ and Z² are each independently CH, CR² or N;
R³ is H, optionally substituted C₁₋₆alkyl or R⁵⁰;
R⁴ is H, optionally substituted C₁₋₆alkyl or R⁵⁰; and
R⁵ is halo, —CN, optionally substituted C₁₋₆alkyl, alkynyl, hydroxy, optionally substituted C₁₋₆alkoxy, nitro, —N(R^{a})₂, —C(O)N(R^{a})₂, —CO₂R^{a} or —C(O)R^{a}.

10. The method according to claim 8 wherein the kinase inhibitor is a pyrimidine-containing compound having a formula II 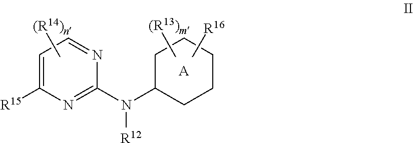
or a pharmaceutically acceptable salt thereof, wherein: 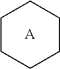
is selected from the group consisting of a six-membered aryl and a six-membered heteroaryl;
n′ is 0, 1 or 2;
m′ iso, 1, 2, 3 or 4;
R¹² is selected from hydrogen, alkyl, alkenyl, alkynyl, haloalkyl, haloalkenyl, haloalkenyl, optionally substituted cycloalkyl, optionally substituted cycloalkylalkyl, optionally substituted cycloalkylalkenyl, optionally substituted cycloalkylalkynyl, optionally substituted aryl, optionally substituted alkyl, optionally substituted alkenyl, optionally substituted alkynyl, —R¹⁹—C(O)OR¹⁷, —R²⁰—N(R¹⁷)R¹⁸ and —R²⁰—OR¹⁷;
each R¹³, when present, is independently selected from the group consisting of optionally substituted alkyl, halo, haloalkyl, cyano, nitro, —OR¹⁷, —N(R¹⁷)₂, —C(O)OR¹⁷ and —C(O)N(R¹⁷)₂,
each R¹⁴, when present, is independently selected from the group consisting of alkyl, halo and haloalkyl;
R¹⁵ is selected from the group consisting of aryl and heteroaryl, where the aryl and the heteroaryl are each independently optionally substituted by one or more substituents selected from the group consisting of oxo, alkyl, halo, haloalkyl, cyano, N-heterocyclyl, N-heteroaryl, aryl, —R¹⁹—OR²¹, —R¹⁹—S(O)_{p′}R²¹ (where p′ is 0, 1 or 2), —R¹⁹—C(O)R²¹, —R¹⁹—C(O)OR²¹, —R¹⁹—C(O)N(R²¹)R²², —R¹⁹—N(R²¹)R²², —R¹⁹—N(R²¹)—R²⁰—N(R²¹)R²², —R¹⁹—N(R²¹)—R²⁰—OR²², —R¹⁹—N(R²¹)C(O)R²², —R¹⁹—N(R²¹)S(O)₂R²², R¹⁹—N(R²¹)C(O)—R¹⁹—N(R²¹)R²², and —R¹⁹—N(R²¹)—R²⁰—N(R²¹)S(O)₂R²², wherein each R²² and R²¹ independently is selected from the group consisting of hydrogen, alkyl, optionally substituted cycloalkyl, optionally substituted heterocyclyl, optionally substituted heterocyclylalkyl, optionally substituted heteroaryl and optionally substituted aryalkyl, and where the N-heterocyclyl, the N-heteroaryl and the aryl are each independently optionally substituted by one or more substituents selected from the group consisting of —C(O)R¹⁷, —R¹⁹—N(R¹⁷)R¹⁸, —R¹⁹—C(O)N(R¹⁷)R¹⁸, alkyl, halo and optionally substituted aryl,
R¹⁶ is an N-heterocyclyl, wherein a nitrogen atom in the N-heterocyclyl is optionally substituted by a substituent selected from the group consisting of alkyl, alkenyl, alkynyl, haloalkyl, haloalkenyl, haloalkynyl, optionally substituted aryl, optionally substituted alkyl, optionally substituted alkenyl, optionally substituted alkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁹—OR¹⁴, —R¹⁹—C(O)R¹⁷, —R¹⁹—C(O)OR¹⁷, —R¹⁹—N(R¹⁷)R¹⁸, —R¹⁹—C(O)N(R¹⁷)R¹⁸, —R¹⁹—C(N═R¹⁷)N(R¹⁷)R¹⁸, —R¹⁹—S(O)₂N(R¹⁷)R¹⁸, and —R¹⁹—S(O)ₜR¹⁷ (where t is 1 or 2); and a carbon atom in the N-heterocyclyl is optionally substituted by a substituent selected from the group consisting of alkyl, alkenyl, alkynyl, halo, haloalkyl, haloalkenyl, haloalkynyl, oxo, optionally substituted aryl, optionally substituted alkyl, optionally substituted alkenyl, optionally substituted alkynyl, optionally substituted cycloalkyl, optionally substituted cycloalkylalkyl, optionally substituted cycloalkylalkenyl, optionally substituted cycloalkylalkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, optionally substituted heteroarylalkynyl, —R¹⁹—OR¹⁷, —R¹⁹—C(O)R¹⁷, —R¹⁹—C(O)OR¹⁷, —R¹⁹—N(R¹⁷)R¹⁸, —R¹⁹—C(O)N(R¹⁷)R¹⁸, —R¹⁹—S(O)₂N(R¹⁷)R¹⁸, and —R¹⁹—S(O)_{p′}R¹⁷ (where p′ is 0, 1 or 2),
each R¹⁷ and each R¹⁸ is independently selected from the group consisting of hydrogen, alkyl, alkenyl, alkynyl, haloalkyl, haloalkenyl, haloalkynyl, optionally substituted cycloalkyl, optionally substituted cycloalkylalkyl, optionally substituted aryl, optionally substituted alkyl, optionally substituted alkenyl, optionally substituted alkynyl, optionally substituted heterocyclyl, optionally substituted heterocyclylalkyl, optionally substituted heterocyclylalkenyl, optionally substituted heterocyclylalkynyl, optionally substituted heteroaryl, optionally substituted heteroarylalkyl, optionally substituted heteroarylalkenyl, and optionally substituted heteroarylalkynyl; or any R¹⁷ and R¹⁸, together with the common nitrogen to which they are both attached, form an optionally substituted N-heteroaryl or an optionally substituted N-heterocyclyl;
each R¹⁹ is independently selected from the group consisting of a direct bond, an optionally substituted straight or branched alkylene chain, an optionally substituted straight or branched alkenylene chain and an optionally substituted straight or branched alkynylene chain; and
each R²⁰ is independently selected from the group consisting of an optionally substituted straight or branched alkylene chain, an optionally substituted straight or branched alkenylene chain and an optionally substituted straight or branched alkynylene chain; provided at least one of R¹⁶ and a substituent on R¹⁵ is a bridged N-heterocyclyl.

11. The method according to claim 8 wherein the kinase inhibitor is a pyrimidine-containing compound having a formula III 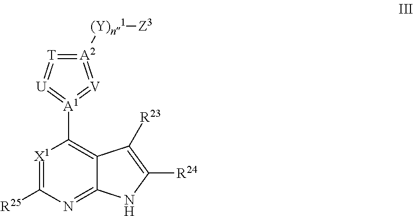
or a pharmaceutically acceptable salt thereof, wherein:
A¹ and A² are independently selected from C and N;
T, U, and V are independently selected from O, S, N, CR²⁷, and NR²⁸;
the 5-membered ring formed by A¹, A², U, T, and V is aromatic;
X¹ is N or CR²⁶;
Y¹ is C₁₋₈ alkylene, C₂₋₈ alkenylene, C₂₋₈ alkynylene, (CR³³R³⁴)_{p″}—(C₃₋₁₀cycloalkylene)-(CR³³R³⁴), (CR³³R³⁴)_{p″}-(arylene)-(CR³³R³⁴)_{q}, (CR³³R³⁴)_{p″}—(C₁₋₁₀heterocycloalkylene)-(CR³³R³⁴)_{q}, (CR³³R³⁴)_{p″}-(heteroarylene)-(CR³³R³⁴)_{q}, (CR³³R³⁴)_{p″}O(CR³³R³⁴), (CR³³R³⁴)_{p″}S(CR³³R³⁴), (CR³³R³⁴)_{p″}C(O)(CR³³R³⁴), (CR³³R³⁴)_{p″}C(O)NR⁴¹(CR³³R³⁴)_{q}, (CR³³R³⁴)_{p″}C(O)O(CR³³R³⁴)_{q}, (CR³³R³⁴)_{p″}OC(O)(CCR³³R³⁴)_{q}, (CR³³R³⁴)_{p″}OC(O)NR⁴¹(CR³³R³⁴)_{q}, (CR³³R³⁴)ₚNR⁴¹(CR³³R³⁴)_{q}, (CCR³³R³⁴)ₚNR⁴¹C(O)NR⁴²(CR³³R³⁴)_{q}, (CR³³R³⁴)_{q}, (CR³³R³⁴)_{p″}S(O)(CR³³R³⁴)_{q}, (CR³³R³⁴)_{p″}S(O)NR⁴¹(CR³³CR³⁴)_{q}, (CR³³R³⁴)_{p″}S(O)₂(CR³³R³⁴)_{q}, or (CR³³R³⁴)_{p″}S(O)₂NR⁴¹(CR³³CR³⁴)_{q}, wherein the C₁₋₈ alkylene, C₂₋₈ alkenylene, C₂₋₈ alkynylene, cycloalkylene, arylene, heterocycloalkylene, or heteroarylene, is optionally substituted with 1, 2, or 3 substituents independently selected from -D¹-D²-D³-D⁴;
Z³ is H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, ═C—R⁴⁹, ═N—R⁴⁹, Cy¹, CN, NO₂, OR³⁵SR³⁵, C(O)R³⁶, C(O)NR⁴¹R⁴², C(O)OR³⁵, OC(O)R³⁶, OC(O)NR⁴¹R⁴², NR⁴¹R⁴², NR⁴¹C(O)R³⁶, NR⁴¹C(O)NR⁴¹R⁴², NR⁴¹C(O)OR³⁵, C(═NR⁴⁹)NR⁴¹R⁴², NR⁴¹C(═NR⁴⁹)NR⁴¹R⁴², S(O)R³⁶, S(O)NR⁴¹R⁴², S(O)₂R³⁶, NR⁴¹S(O)₂R³⁶, C(═NOH)R³⁶, C(═NO(C₁₋₆alkyl)R³⁶, and S(O)₂NR⁴¹R⁴², wherein when Z is H, n″ is 1, or the —(Y)_{n″}—Z moiety is taken together with i) A² to which the moiety is attached, ii) R²⁷ or R²⁸ of either T or V, and iii) the C or N atom to which the R²⁷ or R²⁸ of either T or V is attached to form a 4- to 20-membered aryl, cycloalkyl, heteroaryl, or heterocycloalkyl ring fused to the 5-membered ring formed by A¹, A², U, T, and V, wherein the 4- to 20-membered aryl, cycloalkyl, heteroaryl, or heterocycloalkyl ring is optionally substituted by 1, 2, 3, 4, or 5 substituents independently selected from —(W)_{1n″}-Q;
W is C₁₋₈ alkylenyl, C₂₋₈ alkenylenyl, C₂₋₈ alkynylenyl, O, S, C(O), C(O)NR⁴³, C(O)O, OC(O), OC(O)NR⁴³, NR⁴³, NR⁴³C(O)NR⁴⁴, S(O), S(O)NR⁴³, S(O)₂, or S(O)₂NR⁴³;
Q is H, halo, CN, NO₂, C₁₋₈ alkyl, C₂₋₈ alkenyl, C₂₋₈ alkynyl, C₁₋₈ haloalkyl, halosulfanyl, aryl, cycloalkyl, heteroaryl, or heterocycloalkyl, wherein the C₁₋₈ alkyl, C₂₋₈ alkenyl, C₂₋₈ alkynyl, C₁₋₈ haloalkyl, aryl, cycloalkyl, heteroaryl, or heterocycloalkyl is optionally substituted with 1, 2, 3 or 4 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy², CN, NO₂, OR³⁷, SR³⁷, C(O)R³⁸, C(O)NR⁴³R⁴⁴, C(O)OR³⁷, OC(O)R³⁸, OC(O)NR⁴³R⁴⁴, NR⁴³R⁴⁴, NR⁴³C(O)R³⁸, NR⁴³C(O)NR⁴³R⁴⁴, NR⁴³C(O)OR³⁷, S(O)R³⁸, S(O)NR⁴³R⁴⁴, S(O)₂R³⁸, NR⁴³S(O)₂R³⁸ and S(O)₂NR⁴³R⁴⁴;
Cy¹ and Cy² are independently selected from aryl, heteroaryl, cycloalkyl, and heterocycloalkyl, each optionally substituted by 1, 2, 3, 4 or 5 substituents independently selected from halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, CN, NO₂, —OR³⁹, SR³⁹, C(O)R⁴⁰, C(O)NR⁴⁵R⁴⁶, C(O)OR³⁹, OC(O)R⁴⁰, OC(O)NR⁴⁵R⁴⁶, Nee, NR⁴⁵C(O)R⁴⁰, NR⁴⁵C(O)OR³⁹, NR⁴⁵S(O)R⁴⁰, NR⁴⁵S(O)₂R⁴⁰, S(O)R⁴⁰, S(O)NR⁴⁵R⁴⁶, S(O)₂R⁴⁰, and S(O)₂NR⁴⁵R⁴⁶;
R²³, R²⁴, R²⁵, and R²⁶ are independently selected from H, halo, C₁₋₄ alkyl, C₂₋₄ alkenyl, C₂₋₄ alkynyl, C₁₋₄ haloalkyl, halosulfanyl, aryl, cycloalkyl, heteroaryl, heterocycloalkyl, CN, NO₂, OR²⁹, SR²⁹, C(O)R³⁰, C(O)NR³¹R³², C(O)OR²⁹OC(O)R³⁰, OC(O)NR³¹R³², NR³¹R³², NR³¹C(O)R³⁰, NR³²C(O)OR²⁹, S(O)R³⁰, S(O)NR³¹R³², S(O)₂R³⁰, NR³¹S(O)₂R³⁰, and S(O)₂NR³¹R³²;
R²⁷ is H, halo, C₁₋₄ alkyl, C₂₋₄ alkenyl, C₂₋₄ alkynyl, C₁₋₄ haloalkyl, halosulfanyl, CN, NO₂, OR²⁹, SR²⁹, C(O)R³⁰, C(O)NR³¹R³², C(O)OR²⁹, OC(O)R³⁰, OC(O)NR³¹R³², NR³¹R³², NR³¹C(O)R³⁰, NR³¹C(O)OR²⁹, S(O)R³⁰, S(O)NR³¹R³², S(O)₂R³⁰, NR³¹S(O)₂R³⁰, or S(O)₂NR³¹R³²;
R²⁸ is H, C₁₋₄ alkyl, C₂₋₄ alkenyl, C₂₋₄ alkynyl, C₁₋₄ haloalkyl, OR²⁹, C(O)R³⁰, C(O)NR³¹R³², C(O)OR²⁹, S(O)R³⁰, S(O)NR³¹R³², S(O)₂R³⁰, or S(O)₂NR³¹R³²;
R²⁹ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;
R³⁰ is H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl;
R³¹ and R³² are independently selected from H, C₁₋₁₀ alkyl, C₁₋₆ haloalkyl, C₂₋₆ alkenyl, C₂₋₆ alkynyl, C₁₋₆ alkylcarbonyl, arylcarbonyl, C₁₋₆ alkylsulfonyl, arylsulfonyl, aryl, heteroaryl, cycloalkyl, heterocycloalkyl, arylalkyl, heteroarylalkyl, cycloalkylalkyl and heterocycloalkylalkyl;
or R³¹ and R³² together with the N atom to which they are attached form a 4-, 5-, 6- or 7-membered heterocycloalkyl group;
R³³ and R³⁴ are independently selected from H and -E¹-E²-E³-E⁴;
D¹ and E¹ are independently absent or independently selected from absent or selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆alkyl, C₁₋₆ haloalkyl, C₂₋₈ alkoxyalkyl, C₁₋₆ alkoxy, C₁₋₆haloalkoxy, amino, C₁₋₆ alkylamino, and C₂₋₈ dialkylamino;
D² and E² are independently absent or independently selected from C₁₋₆ alkylene, C₂₋₆ alkenylene, C₂₋₆ alkynylene, (C₁₋₆alkylene)ᵣ-O—(C₁₋₆alkylene)ₛ, (C₁₋₆alkylene)ᵣ-S—(C₁₋₆alkylene)ₛ, (C₁₋₆ alkylene)ᵣ-NR⁴⁷—(C₁₋₆ alkylene)ₛ, (C₁₋₆ alkylene)ᵣ-CO—(C₁₋₆ alkylene)ₛ, (C₁₋₆ alkylene)ᵣ-COO—(C₁₋₆ alkylene)ₛ, (C₁₋₆ alkylene)ᵣ-CONR⁴⁷—(C₁₋₆ alkylene)ₛ, (C₁₋₆ alkylene)ᵣ-SO—(C₁₋₆ alkylene)ₛ, (C₁₋₆ alkylene)ᵣ-SO₂—(C₁₋₆alkylene)ₛ, (C₁₋₆ alkylene)ᵣ-SONR⁴¹—(C₁₋₆alkylene)ₛ, and (C₁₋₆alkylene)ᵣ-NR⁴⁷CONR⁴⁸—(C₁₋₆ alkylene)ₛ, wherein each of the C₁₋₆ alkylene, C₂₋₆ alkenylene, and C₂₋₆ alkynylene is optionally substituted by 1, 2 or 3 substituents independently selected from halo, CN, NO₂, N₃, SCN, OH, C₁₋₆ alkyl, C₁₋₆ haloalkyl, C₂₋₈ alkoxyalkyl, Ĉalkoxy, C₁₋₆ haloalkoxy, amino, C₁₋₆ alkylamino, and C₂₋₈ dialkylamino;
D³ and E³ are independently absent or independently selected from C₁₋₆ alkylene, C₂₋₆ alkenylene, C₂₋₆ alkynylene, arylene, cycloalkylene, heteroarylene, and heterocycloalkylene, wherein each of the C₁₋₆ alkylene, C₂₋₆ alkenylene, C₂₋₆ alkynylene, arylene, cycloalkylene, heteroarylene, and heterocycloalkylene is optionally substituted by 1, 2 or 3 substituents independently selected from halo, CN, NO₂, N₃, SCN, OH, C₁₋₆ alkyl, C₁₋₆haloalkyl, C₂₋₈ alkoxyalkyl, C₁₋₆ alkoxy, C₁₋₆ haloalkoxy, amino, C₁₋₆ alkylamino, and C₂₋₈ dialkylamino;
D⁴ and E⁴ are independently selected from H, halo, C₁₋₄ alkyl, C₂₋₄ alkenyl, C₂₋₄ alkynyl, C₁₋₄ haloalkyl, halosulfanyl, C₁₋₄ hydroxyalkyl, C₁₋₄ cyanoalkyl, Cy¹, CN, NO₂, OR³⁵, SR³⁵, C(O)OR³⁶, C(O)NR⁴¹R⁴², C(O)R³⁵, OC(O)R³⁶, OC(O)NR⁴¹R⁴², NR⁴¹R⁴², NR⁴¹C(O)R³⁶, S(O)NR⁴¹C(O)NR⁴¹R⁴², NR⁴¹C(O)OR³⁵, (═NR⁴⁹)NR⁴¹R⁴², NR⁴¹C(═NR⁴⁹NR⁴¹R⁴², S(O)R³⁶, S(O)NR⁴¹R⁴², NR⁴¹S(O)₂R³⁶, C(═NOH)R³⁶, C(═NO(C₁₋₆ alkyl)R³⁶, and S(O)₂NR⁴¹R⁴², wherein the C₁₋₈ alkyl, C₂₋₈ alkenyl, or C₂₋₈ alkynyl, is optionally substituted with 1, 2, 3, 4, 5, or 6 substituents independently selected from halo, C₁₋₄ alkyl, C₂₋₄ alkenyl, C₂₋₄ alkynyl, C₁₋₄haloalkyl, halosulfanyl, C₁₋₄hydroxyalkyl, C₁₋₄cyanoalkyl, Cy¹, CN, NO₂, OR³⁵, SR³⁵, C(O)R³⁶. C(O)NR⁴¹R⁴², C(O)OR³⁵, OC(O)R³⁶, OC(O)NR⁴¹R⁴², NR⁴¹R⁴², NR⁴¹C(O)R³⁶, NR⁴¹C(O)NR⁴¹R⁴², NR⁴¹C(O)OR³⁵, C(═NR⁴⁹)NR⁴¹R⁴², NR^{c}C(═NR⁴⁹)NR⁴¹R⁴², S(O)R³⁶, S(O)NR⁴¹R⁴², S(O)₂R²⁸, NR⁴¹S(O)₂R³⁶, C(═NOH)R³⁶, C(═NO(C₁₋₆alkyl))R³⁶, and S(O)₂NR⁴¹R⁴²;
R³⁵ is Cy¹, —(C₁₋₆alkyl)-Cy¹, C₁₋₆ alkyl, C₁₋₆ haloalkyl, C₂₋₆ alkenyl, C₂₋₆ alkynyl, wherein the C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆ alkyl, C₁₋₆ haloalkyl, halosulfanyl, aryl, arylalkyl, heteroaryl, heteroarylalkyl, cycloalkyl and heterocycloalkyl;
R³⁶ is H, Cy¹, —(C₁₋₆ alkyl)-Cy′, C₁₋₆ alkyl, C₁₋₆ haloalkyl, C₂₋₆ alkenyl, C₂₋₆ alkynyl, wherein the C₁₋₆ alkyl, C₁₋₆ haloalkyl, C₂₋₆ alkenyl, or C₂₋₆ alkynyl is optionally substituted with 1, 2, or 3 substituents independently selected from OH, CN, amino, halo, C₁₋₆alkyl, C₁₋₆ haloalkyl, C₁₋₆ haloalkyl, halosulfanyl, aryl, arylalkyl, heteroaryl, heteroarylalkyl, cycloalkyl and heterocycloalkyl;
R³⁷ and R³⁹ are independently selected from H, C₁₋₆ alkyl, C₁₋₆ haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein the C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;
R³⁸ and R⁴⁰ are independently selected from H, C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl and heterocycloalkylalkyl, wherein the C₁₋₆alkyl, C₁₋₆haloalkyl, C₂₋₆ alkenyl, C₂₋₆ alkynyl, aryl, cycloalkyl, heteroaryl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆ alkyl, C₁₋₆haloalkyl, C₁₋₆haloalkyl, halosulfanyl, aryl, arylalkyl, heteroaryl, heteroarylalkyl, cycloalkyl and heterocycloalkyl;
R⁴¹ and R⁴² are independently selected from H, Cy¹, —(C₁₋₆alkyl)-Cy¹, C₁₋₁₀ alkyl, C₁₋₆haloalkyl, C₂₋₆ alkenyl, C₂₋₆ alkynyl, wherein the C₁₋₁₀ alkyl, C₁₋₆ haloalkyl, C₂₋₆ alkenyl, or C₂₋₆ alkynyl, is optionally substituted with 1, 2, or 3 substituents independently selected from Cy¹, —(C₁₋₆alkyl)-Cy′, OH, CN, amino, halo, C₁₋₆alkyl, C₁₋₆haloalkyl, C₁₋₆haloalkyl, and halosulfanyl; or
R⁴¹ and R⁴² together with the N atom to which they are attached form a 4-, 5-, 6- or 7-membered heterocycloalkyl group optionally substituted with 1, 2, or 3 substituents independently selected from Cy¹, —(C₁₋₆ alkyl)-Cy¹, OH, CN, amino, halo, C₁₋₆alkyl, C₁₋₆haloalkyl, C₁₋₆haloalkyl, and halosulfanyl;
R⁴³ and R⁴⁴ are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein the C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl; or
R⁴³ and R⁴⁴ together with the N atom to which they are attached form a 4-, 5-, 6- or 7-membered heterocycloalkyl group optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;
R⁴⁵ and R⁴⁶ are independently selected from H, C₁₋₁₀ alkyl, C₁₋₆ haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl and heterocycloalkylalkyl, wherein the C₁₋₁₀ alkyl, C₁₋₆haloalkyl, C₂₋₆ alkenyl, C₂₋₆ alkynyl, aryl, heteroaryl, cycloalkyl, heterocycloalkyl, arylalkyl, heteroarylalkyl, cycloalkylalkyl or heterocycloalkylalkyl is optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl; or
R⁴⁵ and R⁴⁶ together with the N atom to which they are attached form a 4-, 5-, 6- or 7-membered heterocycloalkyl group optionally substituted with 1, 2, or 3 substituents independently selected from OH, CN, amino, halo, C₁₋₆alkyl, C₁₋₆haloalkyl, halosulfanyl, aryl, arylalkyl, heteroaryl, heteroarylalkyl, cycloalkyl and heterocycloalkyl;
R⁴⁷ and R⁴⁸ are independently selected from H and C₁₋₆alkyl;
R⁴⁹ is H, CN, C₁₋₆ alkyl or NO₂;
m″ is 0 or 1;
n″ is 0 or 1;
p″ is 0, 1, 2, 3, 4, 5, or 6;
q is 0, 1, 2, 3, 4, 5, or 6;
r is 0 or 1; and
s is 0 or 1.

12. The method according to claim 8 wherein the kinase inhibitor is a pyrimidine-containing compound having a formula IV 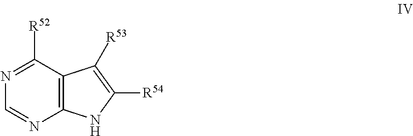
or a pharmaceutically acceptable salt thereof, wherein:
R⁵² is a group of the formula 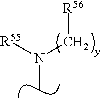
wherein y is 0, 1 or 2 or heteroaryl;
R⁵⁵ is selected from the group consisting of hydrogen, (C₁-C₆)alkyl, (C₁-C₆)alkylsulfonyl, (C₂-C₆)alkenyl, (C₂-C₆)alkynyl wherein the alkyl, alkenyl and alkynyl groups are optionally substituted by deuterium, hydroxy, amino, trifluoromethyl, (C₁-C₄)alkoxy, (C₁-C₆)acyloxy, (C₁-C₆)alkylamino, ((C₁-C₆)alkyl)₂amino, cyano, nitro, (C₂-C₆)alkenyl, (C₂-C₆)alkynyl or (C₁-C₆)acylamino, or R⁵⁵ is (C₃-C₁₀)cycloalkyl wherein the cycloalkyl group is optionally substituted by deuterium, hydroxy, amino, trifluoromethyl, (C₁-C₆)acyloxy, (C₁-C₆)acylamino, (C₁-C₆)alkylamino, ((C₁-C₆)alkyl)₂amino, cyano, cyano(C₁-C₆)alkyl, trifluoromethyl(C₁-C₆)alkyl, nitro, nitro(C₁-C₆)alkyl or (C₁-C₆)acylamino;
R⁵⁶ is (C₂-C₉)heterocycloalkyl wherein the heterocycloalkyl groups must be substituted by one to five carboxy, cyano, amino, deuterium, hydroxy, (C₁-C₆)alkyl, (C₁-C₆)alkoxy, halo, (C₁-C₆)acyl, (C₁-C₆alkylamino), amino(C₁-C₆)alkyl, (C₁-C₆)alkoxy-CO—NH, (C₁-C₆)alkylamino-CO—, (C₂-C₆)alkenyl, (C₂-C₆) alkynyl, (C₁-C₆)alkylamino, amino(C₁-C₆)alkyl, hydroxy(C₁-C₆)alkyl, (C₁-C₆)alkoxy(C₁-C₆)alkyl, (C₁-C₆)acyloxy(C₁-C₆)alkyl, nitro, cyano(C₁-C₆)alkyl, halo(C₁-C₆)alkyl, nitro(C₁-C₆)alkyl, trifluoromethyl, trifluoromethyl(C₁-C₆)alkyl, (C₁-C₆)acylamino, (C₁-C₆)acylamino(C₁-C₆)alkyl, (C₁-C₆)alkoxy(C₁-C₆)acylamino, amino(C₁-C₆)acyl, amino(C₁-C₆)acyl(C₁-C₆)alkyl, (C₁-C₆)alkylamino(C₁-C₆)acyl, ((C₁-C₆)alkyl)₂amino(C₁-C₆)acyl, R⁶⁴R⁶⁵N—CO—O—, R⁶⁴R⁶⁵N—CO—(C₁-C₆)alkyl, (C₁-C₆)alkyl-S(O)_{m′″}, R⁶⁴R⁶⁵NS(O)_{m′″}, R⁶⁴R⁶⁵NS(O)_{m′″}(C₁-C₆)alkyl, R⁶⁴S(O)_{m′″}R⁶⁵N, R⁶⁴S(O)_{m′″}—R⁶⁵N(C₁-C₆)alkyl wherein m is 0, 1 or 2 and R⁶⁴ and R⁶⁵ are each independently selected from hydrogen or a group of the formula 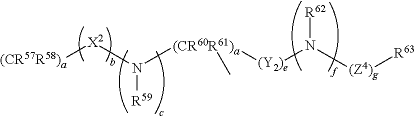
wherein a is 0, 1, 2, 3 or 4;
b, c, e, f and g are each independently 0 or 1;
d is 0, 1, 2, or 3;
X² is S(O)_{n′″} wherein n is 0, 1 or 2, oxygen, carbonyl or —C(═N-cyano)-;
Y² is S(O)_{n′″} wherein n is 0, 1 or 2; or carbonyl;
Z⁴ is carbonyl, C(O)O—, C(O)NR— or S(O)_{n′″} wherein n′″ is 0, 1 or 2;
R⁵⁷, R⁵⁸, R⁵⁹, R⁶⁰, R⁶¹ and R⁶² are each independently selected from the group consisting of hydrogen or (C₁-C₆)alkyl optionally substituted by deuterium, hydroxy, amino, trifluoromethyl, (C₁-C₆)acyloxy, (C₁-C₆)acylamino, ((C₁-C₆)alkyl)₂amino, cyano, cyano(C₁-C₆)alkyl, trifluoromethyl(C₁-C₆)alkyl, nitro, nitro(C₁-C₆)alkyl, or (C₁-C₆)acylamino,
R⁶³ is carboxy, cyano, amino, oxo, deuterium, hydroxy, trifluoromethyl, (C₁-C₆)alkyl, trifluoromethy C₁-C₆alkyl, (C₁-C₆)alkoxy, halo, (C₁-C₆)acyl, (C₁-C₆)alkylamino, ((C₁-C₆)alkyl)₂ amino, amino(C₁-C₆)alkyl, (C₁-C₆)alkoxy-CO—NH—, (C₁-C₆)alkylamino-CO—, (C₂-C₆)alkenyl, (C₂-C₆)alkynyl, (C₁-C₆)alkylamino, hydroxy(C₁-C₆)alkyl, (C₁-C₆)alkoxy(C₁-C₆)alkyl, (C₁-C₆)acyloxy(C₁-C₆)alkyl, nitro, cyano(C₁-C₆)alkyl, halo(C₁-C₆)alkyl, nitro(C₁-C₆)alkyl, trifluoromethyl, trifluoromethyl(C₁-C₆)alkyl, (C₁-C₆)acylamino, (C₁-C₆)acylamino(C₁-C₆)alkyl, (C₁-C₆)alkoxy(C₁-C₆)acylamino, amino(C₁-C₆)acyl, amino(C₁-C₆)acyl(C₁-C₆)alkyl, (C₁-C₆alkylamino(C₁-C₆)acyl, ((C₁-C₆)alkyl)₂amino(C₁-C₆)acyl, R⁶⁴R⁶⁵N—CO—O—, R⁶⁴R⁶⁵N—CO—(C₁-C₆)alkyl, R⁶⁴C(O)NH, R⁶⁴OC(O)NH, R⁶⁴NHC(O)NH, (C₁-C₆)alkyl-S(O)_{m′″}-(C₁-C₆)alkyl, R⁶⁴R⁶⁵NS(O)_{m′″}, R⁶⁴R⁶⁵NS(O)_{m′″}(C₁-C₆alkyl, R⁶⁴S(O)_{m′″}R⁶⁵N, R⁶⁴S(O)_{m′″}R⁶⁵N(C₁-C₆)alkyl wherein m is 0, 1 or 2 and R⁶⁴ and R⁶⁵ are each independently selected from hydrogen or (C₁-C₆)alkyl;
R⁵³ and R⁵⁴ are each independently selected from the group consisting of hydrogen, deuterium, amino, halo, hydroxy, nitro, carboxy, (C₂-C₆)alkenyl, (C₂-C₆)alkynyl, trifluoromethyl, trifluoromethoxy, (C₁-C₆)alkyl, (C₁-C₆alkoxy, (C₃-C₁₀)cycloalkyl wherein the alkyl, alkoxy or cycloalkyl groups are optionally substituted by one to three groups selected from halo, hydroxy, carboxy, amino (C₁-C₆)alkylthio, (C₁-C₆alkylamino, ((C₁-C₆)alkyl)₂amino, (C₅-C₉)heteroaryl, (C₂-C₉)heterocycloalkyl, (C₃-C₉)cycloalkyl or (C₆-C₁₀)aryl, or R⁵³ and R⁵⁴ are each independently (C₃-C₁₀)cycloalkyl, (C₃-C₁₀)cycloalkoxy, (C₁-C₆)alkylamino ((C₁-C₆)alkyl)₂amino, (C₆-C₁₀)arylamino, (C₁-C₆)alkylthio, (C₆-C₁₀)arylthio, (C₁-C₆)alkylsulfinyl, (C₆-C₁₀)arylsulfinyl, (C₁-C₆)alkylsulfonyl, (C₆-C₁₀)arylsulfonyl, (C₁-C₆)acyl, (C₁-C₆alkoxy-CO—NH—, (C₁-C₆)alkyamino-CO—, (C₅-C₉)heteroaryl, (C₂-C₉)heterocycloalkyl or (C₆-C₁₀)aryl wherein the heteroaryl, heterocycloalkyl and aryl groups are optionally substituted by one to three halo, (C₁-C₆)alkyl, (C₁-C₆)alkyl-CO—NH—, (C₁-C₆)alkyoxy-CO—NH—, (C₁-C₆)alkyl-CO—NH—(C₁-C₆)alkyl, (C₁-C₆)alkoxy-CO—NH—(C₁-C₆)alkyl, (C₁-C₆alkoxy-CO—NH—(C₁-C₆)alkoxy, carboxy, carboxy(C₁-C₆)alkyl, carboxy(C₁-C₆)alkoxy, benzyloxycarbonyl(C₁-C₆)alkoxy, (C₁-C₆alkoxycarbonyl(C₁-C₆)alkoxy, (C₆-C₁₀)aryl, amino, amino(C₁-C₆)alkyl, (C₁-C₆)alkoxycarbonylamino, (C₆-C₁₀)aryl(C₁-C₆)alkoxycarbonylamino, (C₁-C₆)alkylamino, ((C₁-C₆)alkyl)₂amino, (C₁-C₆)alkylamino(C₁-C₆)alkyl, ((C₁-C₆)alkyl)₂amino(C₁-C₆)alkyl, hydroxy, (C₁-C₆alkoxy, carboxy, carboxy(C₁-C₆)alkyl, (C₁-C₆alkoxycarbonyl, (C₁-C₆alkoxycarbony C₁-C₆alkyl, (C₁-C₆)alkoxy-CO—NH—, (C₁-C₆)alkyl-CO—NH—, cyano, (C₅-C₉)heterocycloalkyl, amino-CO—NH—, (C₁-C₆)alkylamino-CO—NH—, ((C₁-C₆)alkyl)₂amino-CO—NH—, (C₆-C₁₀)arylamino-CO—NH—, (C₅-C₉)heteroarylamino-CO—NH—, (C₁-C₆)alkylamino-CO—NH—(C₁-C₆)alkyl, ((C₁-C₆)alkyl)₂amino-CO—NH—(C₁-C₆)alkyl, (C₆-C₁₀)arylamino-CO—NH—(C₁-C₆)alkyl, (C₅-C₉)heteroarylamino-CO—NH—(C₁-C₆)alkyl, (C₁-C₆)alkylsulfonyl, (C₁-C₆)alkylsulfonylamino, (C₁-C₆)alkylsulfonylamino(C₁-C₆)alkyl, (C₆-C₁₀)arylsulfonyl, (C₆-C₁₀)arylsulfonylamino, (C₆-C₁₀)arylsulfonylamino(C₁-C₆)alkyl, (C₁-C₆)alkylsulfonylamino, (C₁-C₆)alkylsulfonylamino(C₁-C₆)alkyl, (C₅-C₉)heteroaryl or (C₂-C₉)heterocycloalkyl.

13. The method according to claim 8 wherein the kinase inhibitor is a pyridine-containing compound having a formula V 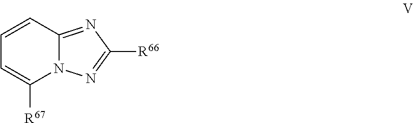
or a pharmaceutically acceptable salt thereof, wherein R⁶⁶ is an amine or an amide and R⁶⁷ is selected from optionally substituted aryl or heteroaryl.

14. The method according to claim 8 wherein the kinase inhibitor is a pyrimidine-containing compound having a formula VI 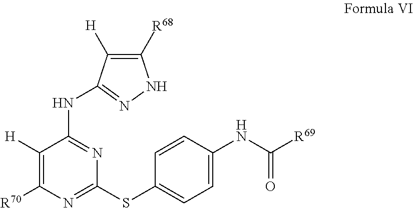
or a pharmaceutically acceptable salt thereof, wherein R⁶⁸ is C₁₋₃alkyl or cyclopropyl, R⁶⁹ is CH₂CH₃, CH₂CF₃, CH₂CH₂CF₃, 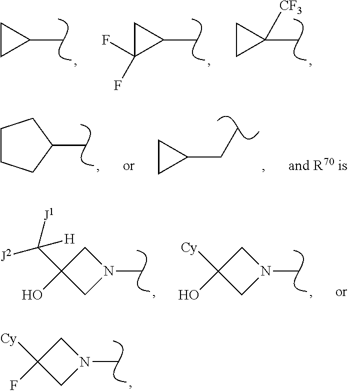
wherein J¹ is H or CH₃, J² is C₁₋₄alkyl, and Cy is C₃₋₅cycloalkyl.

15. The method according to claim 8 wherein the kinase inhibitor is a pyridine-containing compound having a formula VII 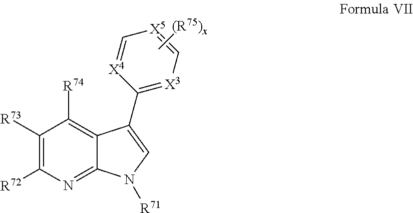
or a pharmaceutically acceptable salt thereof, wherein R⁷¹ is T¹-R′ or is —Si(R′)₃;
R⁷², R⁷³, and R⁷⁴ are each independently halogen, CN, NO₂, or V¹—R′;
X³, X⁴ and X⁵ are each independently N, or CH, wherein the hydrogen atom of CH is optionally replaced by R⁷⁵;
x is 1, 2, 3, or 4;
each occurrence of R⁷⁵ is independently halogen, CN, NO₂, or U¹—R′;
T¹, V¹, and U¹ are each independently a bond or an optionally substituted C₁-C₆ alkylidene chain, wherein up to two methylene units of the chain are optionally and independently replaced by —NR′—, —S—, —O—, —CS—, —CO₂—, —OCO—, —CO—, —COCO—, —CONR′—, —NR′CO—, —NR′CO₂—, —SO₂NR′—, —NR′SO₂—, —CONR′NR′—, —NR′CONR′—, —OCONR′—, —NR′NR′—, —NR′SO₂NR′—, —SO—, —SO₂—, —PO—, —PO₂—, or —POR′—; and
each occurrence of R′ is independently hydrogen or an optionally substituted group selected from a C₁-C₆ aliphatic group, a 3-8-membered saturated, partially unsaturated, or fully unsaturated monocyclic ring having 0-3 heteroatoms independently selected from nitrogen, oxygen, or sulfur, or an 8-12 membered saturated, partially unsaturated, or fully unsaturated bicyclic ring system having 0-5 heteroatoms independently selected from nitrogen, oxygen, or sulfur; or
two occurrences of R′ are taken together with the atom(s) to which they are bound to form an optionally substituted 3-12 membered saturated, partially unsaturated, or fully unsaturated monocyclic or bicyclic ring having 0-4 heteroatoms independently selected from nitrogen, oxygen, or sulfur; or
two occurrences of R′ that form a ring are on a single substituent and form a monocyclic or bicyclic; or
two occurrences of R′ are on two substituents and form a bicyclic fused ring with the ring to which the R⁷⁵ substituents are attached.

16. The method according to claim 8 wherein the kinase inhibitor is a pyridine-containing compound having a formula VIII 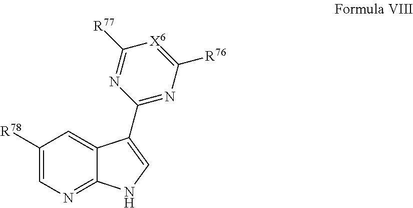
or a pharmaceutically acceptable salt thereof, wherein:
R⁷⁸ is H, Cl or F;
X⁶ is N or CR⁷⁹;
R⁷⁷ is H, F, R′, OH, OR′, COR′, COOH, COOR′, CONH₂, CONHR′, CON(R′)₂, or CN;
R⁷⁹ is H, F, R′, OH, OR′, COR′, COOH, COOR′, CONH₂, CONHR′, CON(R′)₂, or CN;
or R⁷⁷ and R⁷⁹, taken together, form a 5-7 membered aryl or heteroaryl ring optionally substituted with 1-4 occurrences of R⁸⁰;
R′ is a C₁₋₃aliphatic optionally substituted with 1-4 occurrences of R⁸¹;
each R⁸¹ is independently selected from halogen, CF₃, OCH₃, OH, SH, NO₂, NH₂, SCH₃, NCH₃, CN or unsubstituted C₁₋₂ aliphatic, or two R⁸¹ groups, together with the carbon to which they are attached, form a cyclopropyl ring or C═O;
each R⁸⁰ is independently selected from halogen, OCH₃, OH, NO₂, NH₂, SH, SCH₃, NCH₃, CN or unsubstituted C₁₋₂aliphatic;
R⁷⁶ is 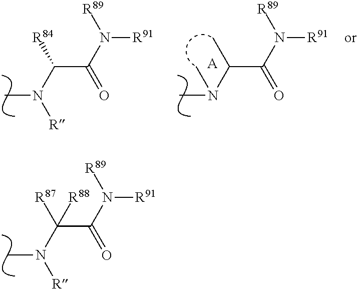
R″ is H or is a —C₁₋₂ aliphatic optionally substituted with 1-3 occurrences of R⁸²;
each R⁸² is independently selected from halogen, OCH₃, OH, SH, NO₂, NH₂, SCH₃, NCH₃, CN, CON(R⁸³)₂ or unsubstituted C₁₋₂ aliphatic, or two R⁸² groups, together with the carbon to which they are attached, form a cyclopropyl ring, or C═O;
R⁸⁴ is a C₁₋₄ aliphatic optionally substituted with 1-5 occurrences of R⁸⁵;
each R⁸⁵ is independently selected from halogen, OCH₃, OH, NO₂, NH₂, SH, SCH₃, NCH₃, CN or unsubstituted C₁₋₂aliphatic, or two R⁸⁵ groups, together with the carbon to which they are attached, form a cyclopropyl ring;
ring A is a 4-8 membered saturated nitrogen-containing ring comprising up to two additional heteroatoms selected from N, O, or S and optionally substituted with 1-4 occurrences of R⁸⁶;
each R⁸⁶ is independently selected from halogen, R′, NH₂, NHR′, N(R)₂, SH, SR′, OH, OR′, NO₂, CN, CF₃, COOR′, COOH, COR′, OC(O)H, OC(O)R′, CONH₂, CONHR′, CON(R′)₂, NHC(O)R′ or NR′C(O)R′; or any two R⁸⁶ groups, on the same substituent or different substituents, together with the atom(s) to which each R⁸⁶ group is bound, form a 3-7 membered saturated, unsaturated, or partially saturated carbocyclic or heterocyclic ring optionally substituted with 1-3 occurrences of R⁸¹;
R⁸⁷ is C₁₋₄ aliphatic optionally substituted with 1-5 occurrences of R⁸⁵;
R⁸⁸ is C₁₋₂ alkyl; or
R⁸⁷ and R⁸⁸ are taken together to form a 3-7 membered carbocyclic or heterocyclic saturated ring optionally substituted with 1-5 occurrences of R⁸⁵;
R⁸⁹ is H or unsubstituted C₁₋₂ alkyl;
R⁹⁰ is H or unsubstituted C₁₋₂ alkyl; and
R⁹¹ is a C₂₋₃ aliphatic or cycloaliphatic optionally substituted with up to 6 occurrences of F.

17. The method according to claim 1 wherein the kinase inhibitor is selected from the group consisting of:

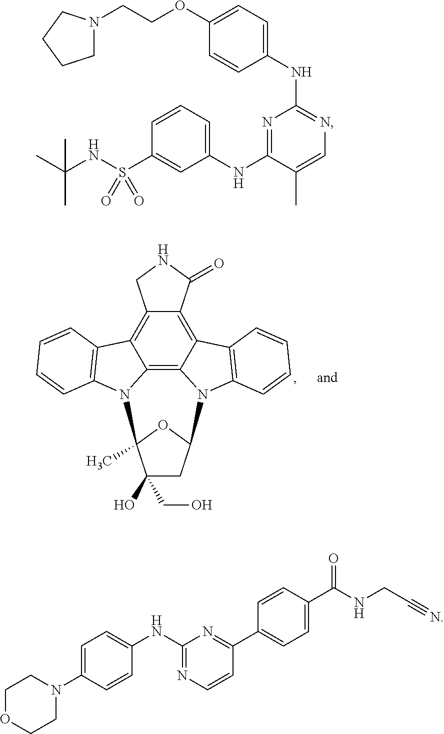 [SMILES: Cc1cnc(Nc2ccc(OCCN3CCCC3)cc2)nc1Nc1cccc(S(=O)(=O)NC(C)(C)C)c1 ; C[C@]12O[C@H](C[C@]1(O)CO)n1c3ccccc3c3c4c(c5c6ccccc6n2c5c31)CNC4=O ; N#CCNC(=O)c1ccc(-c2ccnc(Nc3ccc(N4CCOCC4)cc3)n2)cc1]

18. The method according to claim 1 wherein the kinase inhibitor is cyclopropanecarboxylic acid {5-[4-(1,1-dioxo-1-thiomorpholin-4ylmethyl)-phenyl]-[1,2,4]triazaolo[1,5-a]pyridine-2-yl}-amide, or a salt thereof.

19. The method according to claim 1 wherein the kinase inhibitor is 3-{(3R,4R)-4-methyl-3-[methyl-(7H-pyrrolo[2,3-d]pyrimidin-4-yl)-amino]-piperidin-1-yl}-3-oxo-propionitrile or a salt thereof.

20. The method according to claim 1 wherein the kinase inhibitor is 2-(3-(4-(7H-pyrrolo[2,3-d]pyrimidin-4-yl)-1H-pyrazol-1-yl)-1-(ethylsulfonyl)azetidin-3-yl)acetonitrile or a salt thereof.

21. The method according to claim 1 wherein the kinase inhibitor is selected from N2-(3,4,5-trimethyl)phenyl-5-methyl-N4-(2-oxo-2,3-dihydro-1,3-benzoxazol-5-yl)-2,4-pyrimidinediamine, 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine bis-sodium salt, 5-methyl-N4-[3-(phosphonooxy)methyl-2-oxo-2,3-dihydro-1,3-benzoxazol-5-yl]-N2-(3,4,5-trimethyl)phenyl-2,4-pyrimidinediamine, N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine bis-sodium salt, N2-(3-methoxy-5-trifluoromethyl)phenyl-5-methyl-N4-[3-(phosphonooxy)methyl-2-oxo-2,3-dihydro-1,3-benzoxazol-5-yl]-2,4-pyrimidinediamine, 5-(2-(3-methoxy-4,5-dimethylphenylamino)-5-methylpyrimidin-4-ylamino)benzo[d]oxazol-2(3H)-one, (5-(2-(4-fluoro-3-methoxy-5-methylphenylamino)-5-methylpyrimidin-4-ylamino)-2-oxobenzo[d]oxazol-3(2H)-yl)methyl dihydrogen phosphate, 5-(2-(4-fluoro-3-methoxy-5-methylphenylamino)-5-methylpyrimidin-4-ylamino)benzo[d]oxazol-2(3H)-one, or a salt thereof.

22. The method according to claim 1, further comprising:
administering to the subject a first dose of the kinase inhibitor, or composition comprising the kinase inhibitor;
determining a therapeutic blood level of the kinase inhibitor in the subject, or a therapeutic metabolite blood level of the kinase inhibitor, in the subject;
adjusting the first dose to a second dose; and
and administering the second dose.

23. The method according to claim 1 wherein a single kinase inhibitor is administered serially in plural administrations to the subject.

24. The method according to claim 1 wherein two or more kinase inhibitors are administered either serially or in combination to the subject.

25. The method according to claim 1 wherein the kinase inhibitor, or composition comprising the kinase inhibitor, is administered by a method selected from oral, buccal, mucosal, sublingual, intravenous, intra-arterial, intramuscular, subcutaneous, intraperitoneal, intraarticular, infusion, intrathecal, intraurethral, topical, subdermal, transdermal, intranasal, inhalation, pulmonary tract, intratracheal, intraocular, ocular, intraaural, vaginal, and rectal.

26. The method according to claim 1 wherein the kinase inhibitor is administered prophylactically.

27. The method according to claim 1 further comprising administering a second therapeutic agent to the subject prior to administering the kinase inhibitor, subsequent to administering the kinase inhibitor, or in combination with, or adjunctively to, the kinase inhibitor.

28. The method according to claim 26 wherein the second therapeutic agent is selected from an analgesic, an antibiotic, an anticoagulant, an anti-delirium agent, an anti-inflammatory, a bronchodilator, a hypnotic, a sedative, a neuromuscular blocking agent, an antihistamine, or combinations thereof.

29. The method according to claim 8 comprising continuously administering the compound, or a composition comprising the compound, to the subject.

30. A method for treating a subject who is on, or is about to be placed on, a ventilator, comprising administering to the subject the kinase inhibitor of claim 8 in an amount effective to inhibit ventilator-associated muscular atrophy.

31. The method according to claim 30, comprising administering to a subject the kinase inhibitor in an amount effective to reduce or inhibit injury of diaphragm muscle fibers.

32. The method according to claim 30 wherein administering comprises:
exposing the subject to a first dose of the kinase inhibitor; and
adjusting the first dose to a second dose by considering the effects of the first dose; and
administering the second dose optimized to inhibit and/or prevent injury of diaphragm muscle fibers.

33. The method according to claim 30 wherein the kinase inhibitor is administered in an amount and at time effective to reduce weaning time from a ventilator relative to a weaning time if the subject did not receive the kinase inhibitor.

34. The method according to claim 30 for weaning a subject off a ventilator, comprising:
administering to the subject the kinase inhibitor in an amount effective to promote diaphragm muscle activity;
removing the ventilator from the subject; and
administering a second dose of the kinase inhibitor.

35. The method according to claim 30 further comprising administering a second therapeutic.

36. A composition formulated for reducing or substantially preventing muscular degradation, comprising the kinase inhibitor according to claim 8.

37. The composition according to claim 36 comprising an excipient, a second kinase inhibitor, an additional therapeutic agent, or combinations thereof.
